(19) United States
(12) Patent Application Publication
(10) Pub. No.: US 2025/0145950 A1
(43) Pub. Date: May 8, 2025

(54) Methods for Activation and Expansion of T Cells

(71) Applicant: Alaunos Therapeutics, Inc., Houston, TX (US)

(72) Inventors: Drew Caldwell DENIGER, Houston, TX (US); Lenka Victoria HURTON, Houston, TX (US); Donghyun JOO, Houston, TX (US); Yaoyao SHI, Houston, TX (US); An LU, Houston, TX (US); Victor CARPIO, Houston, TX (US); Matthew COLLINSON-PAUTZ, Houston, TX (US)

(73) Assignee: Alaunos Therapeutics, Inc., Houston, TX (US)

(21) Appl. No.: 18/834,112

(22) PCT Filed: Feb. 1, 2023
(86) PCT No.: PCT/US2023/061765

§ 371 Date: Jul. 29, 2024

Related U.S. Application Data
(60) Provisional application No. 63/267,424, filed on Feb. 1, 2022.

(51) Int. Cl. C12N 5/0783; C12N 15/85

(52) U.S. Cl. CPC C12N 5/0636; C12N 15/85; C12N 2500/32; C12N 2501/2302; C12N 2501/2307; C12N 2501/2312; C12N 2501/2315; C12N 2501/2321; C12N 2510/00

(57) ABSTRACT

Methods for culturing and expanding lymphocytes using unconventional cytokines are provided. These methods include techniques for culturing and expanding lymphocytes using streamlined approaches, including approaches using agonists for stimulation, and approaches more suitable for clinical manufacturing. Compositions of expanded populations of lymphocytes are also provided.

### INCORPORATION OF SEQUENCE LISTING

This application is a U.S. National Phase application of International Application No. PCT/US2023/061765, filed Feb. 1, 2023, which claims the benefit of U.S. Provisional Application No. 63/267,424, filed Feb. 1, 2022, the entire contents of each of which are incorporated herein by reference. A sequence listing contained in the file named P35189WO00_SL.XML, which is 2,206,103 bytes (measured in MS-Windows®) and was created on Feb. 1, 2023, is filed electronically herewith and incorporated by reference in its entirety.

### FIELD

The instant disclosure relates to methods and compositions for activation and/or expansion of electroporated lymphocyte populations, e.g., electroporated T cells.

### BACKGROUND

Lymphocytes, in particular T cells are increasingly engineered for use in treatment of various pathologies. In order to use engineered T cells therapeutically, primary cells need to be transfected and cultured and often further expanded while maintaining the genetic alterations made to the cells. Methods exist for expansion and manipulation of T cells, but methods that can more reliably produce more cells quickly are needed to improve cell therapy.

### SUMMARY

The instant disclosure provides methods for culturing and expanding lymphocytes, in particular T cells, using unconventional culture components. In some embodiments, the T cells have been genetically modified.

Provided herein is a method of expanding a population of electroporated T cells comprising culturing the electroporated T cells with a first culture medium comprising one or more cytokines. In some embodiments, the electroporated T cells are contacted with the first culture medium within 12 hours of electroporation. In some embodiments, the one or more cytokines are selected from the group consisting of IL-7, IL-15, and IL-21. In some embodiments, the first culture medium further comprises an exogenous glutathione precursor. In some embodiments, the glutathione precursor is N-acetylcysteine (NAC). In some embodiments, the first culture medium comprises IL-15. In some embodiments, the first culture medium comprises IL-7. In some embodiments, the first culture medium comprises IL-21. In some embodiments, the first culture medium comprises IL-7 and IL-21.

Also provided herein is a method of expanding a population of electroporated T cells comprising culturing the electroporated T cells with a first culture medium comprising an exogenous glutathione precursor and IL-15, wherein the electroporated T cells are contacted with the first culture medium within 12 hours of electroporation. In some embodiments, the glutathione precursor is N-acetylcysteine (NAC). In some embodiments, the first culture medium comprises IL-7. In some embodiments, the first culture medium comprises IL-21. In some embodiments, the first culture medium comprises IL-7 and IL-21.

In some embodiments, the electroporated T cells were electroporated prior to culturing with the first culture medium. In some embodiments, the electroporated T cells are cultured in the first culture medium for 6-12 hours after electroporation.

In some embodiments, the method also includes culturing the T cells with a second culture medium, wherein the second culture medium comprises one or more cytokines selected from the group consisting of IL-7, IL-12, and IL-21. In some embodiments, the second culture medium comprises IL-7, IL-12, and IL-21.

In some embodiments, IL-21 is added to the second culture medium every 2 to 3 days. In some embodiments, at least one of the cytokines selected from the group consisting of IL-7 and IL-12 are added to the second culture medium only on the first day of culturing. In some embodiments, IL-7 and IL-12 are added to the second culture medium only on the first day of culturing. In some embodiments, the T cells are cultured in the second culture medium after being cultured in the first culture medium. In some embodiments, the T cells are cultured in the second culture medium for 11 to 13 days.

In some embodiments, the method also includes culturing the T cells with a third culture medium, wherein the third culture medium comprises one or more cytokines selected from the group consisting of IL-2 and IL-21. In some embodiments, the third culture medium comprises IL-2. In some embodiments, the third culture medium comprises IL-21. In some embodiments, the third culture medium further comprises IL-12. In some embodiments, the third culture medium further comprises an exogenous glutathione precursor. In some embodiments, the exogenous glutathione precursor in NAC. In some embodiments, the third culture medium comprises IL-12, IL-21 and NAC. In some embodiments, the third culture medium comprises IL-2, IL-12, IL-21 and NAC.

In some embodiments, IL-21 is added to the third culture medium every 2 to 3 days. In some embodiments, IL-2 is added to the third culture medium every 3 to 4 days. In some embodiments, IL-2 is present in the third culture medium in an amount from 30 U/ml to 3000 U/ml. In some embodiments, the IL-12 is added to the third culture medium only on the first day of culturing. In some embodiments, the T cells are cultured in the third culture medium after being cultured in the second culture medium. In some embodiments, the T cells are cultured in the third culture medium for 11 to 13 days.

In some embodiments, the T cells are cultured in the third culture medium after being cultured in the second culture medium. In some embodiments, the T cells are cultured in the third culture medium for 11 to 13 days.

In some embodiments, the first, second and/or third culture media further comprise a TCR agonist. In some embodiments, the TCR agonist is a CD3 agonist. In some embodiments, the first, second and/or third culture media further comprise an agonist of a T cell costimulatory molecule. In some embodiments, the agonist of a T cell costimulatory molecule is a CD28 agonist.

In some embodiments, the first, second and/or third culture media further comprise a nanomatrix. In some embodiments, the TCR agonist and/or the T cell costimulatory molecule is associated with the nanomatrix.

In some embodiments, the method also includes culturing the cells with feeder cells.

Also provided herein is a population of engineered T cells manufactured according to any of the methods described above or herein. In some embodiments, more than 10% of the engineered T cells in the population comprise one or more of the following: an exogenous TCR or functional fragment thereof, and an exogenous membrane-bound IL-15. In some embodiments, more than 2% of the engineered T cells in the population co-express an exogenous TCR or functional fragment thereof and an exogenous membrane-bound IL-15.

In some embodiments, more than 10% of the engineered T cells in the population comprise an exogenous TCR or functional fragment thereof, and wherein more than 20% of the population of engineered T cells are CCR7+/CD45RO+. In some embodiments, more than 10% of the engineered T cells in the population comprise an exogenous TCR or functional fragment thereof, and wherein more than 40% of the population of engineered T cells are CD95+/CD62L+. In some embodiments, the population of engineered T cells comprise more than 5%, 10%, 15%, 20%, 25%, 30%, 35%, 40%, 45% or 50% CD45RA+CD45RO-CD62L+CD95+ cells. In some embodiments, the population of engineered T cells comprise more than 5%, 10%, 15%, 20%, 25%, 30%, 35%, 40%, 45% or 50% CD45RA+CD45RO+CD62L+CD95+ cells.

Provided herein is a population of cells comprising a polycistronic expression cassette comprising a first cistron comprising a polynucleotide sequence that encodes a fusion protein that comprises IL-15, or a functional fragment or functional variant thereof, and IL-15Rα, or a functional fragment or functional variant thereof; a second cistron comprising a polynucleotide sequence that encodes a TCR beta chain comprising a Vβ region and a Cβ region; and a third cistron comprising a polynucleotide sequence that encodes a TCR alpha chain comprising a Vα region and a Cα region. In some embodiments, the population of cells are T cells that comprise more than 5%, 10%, 15%, 20%, 25%, 30%, 35%, 40%, 45% or 50% CD45RA+CD45RO-CD62L+CD95+ cells. In other embodiments, the population of cells are T cells that comprise more than 5%, 10%, 15%, 20%, 25%, 30%, 35%, 40%, 45% or 50% CD45RA+CD45RO+CD62L+CD95+ cells.

Also provided herein is a population of cells comprising a polycistronic expression cassette comprising a first cistron comprising a polynucleotide sequence that encodes a fusion protein that comprises IL-15, or a functional fragment or functional variant thereof, and IL-15Rα, or a functional fragment or functional variant thereof, a second cistron comprising a polynucleotide sequence that encodes a TCR beta chain comprising a Vβ region and a Cβ region; and a third cistron comprising a polynucleotide sequence that encodes a TCR alpha chain comprising a Vα region and a Cα region, wherein the population of cells are T cells that comprise more than 5%, 10%, 15%, 20%, 25%, 30%, 35%, 40%, 45% or 50% CD45RA+CD45RO-CD62L+CD95+ cells.

Also provided herein is a population of cells comprising a polycistronic expression cassette comprising a first cistron comprising a polynucleotide sequence that encodes a fusion protein that comprises IL-15, or a functional fragment or functional variant thereof, and IL-15Rα, or a functional fragment or functional variant thereof, a second cistron comprising a polynucleotide sequence that encodes a TCR beta chain comprising a Vβ region and a Cβ region; and a third cistron comprising a polynucleotide sequence that encodes a TCR alpha chain comprising a Vα region and a Cα region, wherein the population of cells are T cells that comprise more than 5%, 10%, 15%, 20%, 25%, 30%, 35%, 40%, 45% or 50% CD45RA+CD45RO+CD62L+CD95+ cells.

### BRIEF DESCRIPTION OF THE DRAWINGS

The accompanying drawings which are incorporated in and constitute a part of this specification, illustrate exemplary embodiments of the present disclosure and, together with the description, serve to explain principles of the present disclosure.

FIG. 1 is a set of schematics of the structures of TCRα (A), TCRβ (B), and mbIL15 (15), shown from N terminus (left) to C terminus (right).

FIG. 2A is a set of schematics of the ORFs of tricistronic Cassettes APBT15, ATBP15, AP15 TB, AT15PB, BPAT15, BTAP15, BP15TA, and BT15PA. FIG. 2B is a set of schematics of the ORFs of control Cassettes 15, APB, and BPA.

FIG. 3 is a schematic diagram depicting double transposition and single transposition approaches using a Sleeping Beauty transposon/transposase system to generate T cells expressing TCRα/TCRβ and mbIL15.

FIG. 4 is a set of 2-parameter flow plots showing transgene co-expression as assessed after electroporation and overnight incubation for each of Groups 1-14.

FIG. 5A-5C shows TCR and mbIL15 expression after electroporation in various recovery media (Day 1). FIG. 5A provides representative TCR and mbIL15 expression data from cells cultured overnight with recovery media containing different cytokines+/−N-acetylcysteine (NAC). FIG. 5B provides TCR expression data from four donors presented as % mTCR+ cells out of CD3+ cells. FIG. 5C provides TCR and mbIL15 co-expression data from four donors presented as % TCR+mbIL15+ cells out of CD3+ cells.

FIG. 6A-6C shows TCR and mbIL15 expression after first phase expansion in various first expansion media (Day 13). FIG. 6A provides representative TCR and mbIL15 expression data from cells expanded with first expansion media containing a TCR and co-stimulation agonist and different cytokines. FIG. 6B provides TCR expression data from two donors presented as % mTCR+ cells out of CD3+ cells. FIG. 6C provides TCR and mbIL15 co-expression data from two donors presented as % TCR+mbIL15+ cells out of CD3+ cells. *indicates the cytokine was added on the first day of the expansion phase only.

FIG. 7A-7C shows TCR and mbIL15 expression after first phase expansion in various first expansion media (Day 13). FIG. 7A provides representative TCR and mbIL15 expression data from cells expanded with first expansion media containing a TCR and co-stimulation agonist different cytokines. FIG. 7B provides TCR expression data from three donors presented as % mTCR+ cells out of CD3+ cells. FIG. 7C provides TCR and mbIL15 co-expression data from three donors presented as % TCR+mbIL15+ cells out of CD3+ cells. *indicates the cytokine was added on the first day of the expansion phase only.

FIG. 8A-8C shows TCR and mbIL15 expression after second phase expansion in various second expansion media (Day 13). FIG. 8A provides representative TCR and mbIL15 expression data from cells expanded with second expansion media a TCR and co-stimulation agonist and different cytokines in differing concentrations+/−NAC. FIG. 8B provides TCR expression data from three donors presented as % mTCR+ cells out of CD3+ cells. FIG. 8C provides TCR and mbIL15 co-expression data from three donors presented as % TCR+mbIL15+ cells out of CD3+ cells. *indicates the cytokine was added on the first day of the expansion phase only.

FIG. 9 shows the fold expansion of T cells during second phase expansion in varying second phase expansion media containing a TCR and co-stimulation agonist and different cytokines in differing concentrations+/−NAC.

FIG. 10A is a set of 2-parameter flow plots showing representative TCR transgene expression in CD3+ cells after overnight incubation for each of Groups 1-14. FIG. 10B provides TCR expression data from three donors presented as % mTCR+ cells out of CD3+ cells.

FIG. 11A-11C shows TCR and mbIL15 expression after first phase expansion (Day 13). FIG. 11A provides representative TCR and mbIL15 expression data from each of Groups 1-14. FIG. 11B provides TCR expression data from three donors presented as % mTCR+ cells out of CD3+ cells. FIG. 11C provides TCR and mbIL15 co-expression data from three donors presented as % TCR+mbIL15+ cells out of CD3+ cells.

FIG. 12A-12B shows total numbers of TCR+ and TCR+mbIL15+ cells after first phase expansion (Day 13). FIG. 12A provides TCR expression data from three donors presented as total number of mTCR+ T cells. FIG. 12B provides total number of TCR+mbIL15+ T cells from three donors.

FIG. 13A-13B shows cell viability after electroporation (Day 1; FIG. 13A) and after first phase expansion (Day 13; FIG. 13B) for each of Groups 1-14.

FIG. 14A-14B shows specific induction of activation marker, 4-1BB, after overnight co-culture of transposed T cells from each of Groups 1-14 after first phase expansion (Day 13) with wild-type or mutant neoantigen pulsed T2 cells. Data is presented as % 4-1BB positive cells of CD8+ cells at increasing concentrations of neoantigen peptide.

FIG. 15 shows phosphorylated STAT5 levels in transposed CD3+ T cells from each of Groups 1-14 after first phase expansion (Day 13). Isotype negative control and IL-15 treated positive control was included for comparison. (dTp=double transposed with separate mbIL15 and TCR vectors).

FIG. 16 shows apoptosis levels in transposed T cells from each of Groups 2-14 after being expanded for 13 days and then activated for 9 days with CD3/CD28 Dynabeads® (ThermoFisher).

FIG. 17 is a set of schematics illustrating the differences between the S version and N version of the TCR only and mbIL15 TCR constructs shown from N terminus (left) to C terminus (right).

FIG. 18 shows phosphorylated STAT5 levels after second phase expansion (Day 27) in CD3+ T cells transposed with different versions of polycistronic plasmids encoding TCR001. Some containing non-cysteine substituted TCR constant regions (N version) or that are optionally further codon-optimized (NU version). Non-transposed (NT)=NT (Group 2.1); BPA (Group 2.2); BPA-N (Group 2.3); AP15 TB (Group 2.4); AP15 TB-N (Group 2.5); AP15 TB-NU (Group 2.6); BP15TA (Group 2.7); BP15TA-N (Group 2.8); and BP15TA-NU (Group 2.9).

FIG. 19A-19B shows functional data from transposed T cells co-cultured with neoantigen pulsed dendritic cells. FIG. 19A shows specific induction of activation marker, 4-1BB, after overnight co-culture of transposed T cells from each of Groups 2.1-2.9 after second phase expansion (Day 27) with wild-type or mutant neoantigen peptide pulsed dendritic cells. Data is presented as % 4-1BB positive cells of CD8+ cells at increasing concentrations of neoantigen peptide. FIG. 19B shows interferon-7 (IFN-γ) secretion after overnight co-culture of transposed T cells from each of Groups 2.1-2.9 after second phase expansion (Day 27) with wild-type or mutant neoantigen pulsed dendritic cells.

FIG. 20A-20B shows TCR expression and cell survival after 4 weeks of long-term cytokine withdrawal (LTWD) incubation in transduced cells from each of Groups 2.2-2.9. FIG. 20A shows the expression of mTCR detected on CD3+ gated population with mouse TCR beta antibody and FIG. 20B shows cell survival as the percent of live cells recovered relative to initial input number of cells at the beginning of the LTWD.

FIG. 21A-21B shows specific induction of activation marker, 4-1BB, after overnight co-culture of transposed T cells from each of Groups 2.2-2.9 after 4 weeks of LTWD incubation with wild-type or mutant neoantigen (10 μg/ml) pulsed dendritic cells.

FIG. 22A-22B shows IFN-γ secretion after overnight co-culture of transposed T cells from each of Groups 2.2-2.9 after 4 weeks of LTWD incubation with wild-type or mutant neoantigen (10 μg/ml) pulsed dendritic cells.

FIG. 23A-23C is a set of pie charts showing the mean frequency of live CD3⁺ T cell memory and effector subsets at day 11 post-expansion (FIG. 23A), day 22 post-expansion (FIG. 23B), and after 4 weeks of LTWD culture (FIG. 23C) in cells transposed with the tested plasmids (Groups 2.2-2.9).

FIG. 24 is a set of 2-parameter flow plots showing representative TCR and mbIL15 transgene co-expression in CD3+ cells after overnight incubation (Day 1), after first phase expansion (Day 11, pre- and post-enrichment) and after second phase expansion (Day 22) for each of Groups 3.2-3.4 expressing TCR001+/−mbIL15 (BPA-N, AP15 TB-NU, and BP15TA-NU).

FIG. 25A-25C shows TCR+ population changes during the first expansion phase (Day 1 vs. Day 11 pre-enrichment) for cells transposed with various TCRs+/−mbIL15 (Groups 3.1-3.30). Each graph presents data from a separate TCR; TCR only=BPA-N, TCR with mbIL15=AP15 TB-NU or BP15TA-NU.

FIG. 26A-26C shows TCR+ population changes during the second expansion phase (Day 11 post-enrichment vs. Day 22) for cells transposed with various TCRs+/−mbIL15 (Groups 3.1-3.30). Each graph presents data from a separate TCR; TCR only=BPA-N, TCR with mbIL15=AP15 TB-NU or BP15TA-NU.

FIG. 27A-27C shows TCR+/mbIL15+ population changes during the first expansion phase (Day 1 vs. Day 11 pre-enrichment) for cells transposed with various TCRs+/−mbIL15 (Groups 3.1-3.30). Each graph presents data from a separate TCR; =BPA-N, TCR with mbIL15=AP15 TB-NU or BP15TA-NU.

FIG. 28A-28C shows TCR+/mbIL15+ population changes during the second expansion phase (Day 11 post-enrichment vs. Day 22) for cells transposed with various TCRs+/−mbIL15 (Groups 3.1-3.30). Each graph presents data from a separate TCR; =BPA-N, TCR with mbIL15=AP15 TB-NU or BP15TA-NU.

FIG. 29A-29I shows specific induction of activation marker, 4-1BB, after overnight co-culture of transposed T cells from each of Groups 3.1-3.30 after second phase expansion (Day 27) with wild-type (WT) or mutant (Mut) neoantigen pulsed dendritic cells. Data is presented as % 4-1BB positive cells of CD8+ cells at increasing concentrations of neoantigen peptide. NT=non-transposed; TCR only=BPA-N, TCR with mbIL15=AP15 TB-NU or BP15TA-NU.

FIG. 30A-30I shows IFN-γ secretion after overnight co-culture of transposed T cells from each of Groups 3.1-3.30 after second phase expansion (Day 27) with wild-type (WT) or mutant (Mut) neoantigen pulsed dendritic cells. Data is presented as IFN-γ level (pg/ml) at increasing concentrations of neoantigen peptide. NT=non-transposed; TCR only=BPA-N, TCR with mbIL15=AP15 TB-NU or BP15TA-NU.

FIG. 31 shows the specific lysis of negative control (Mut+HLA−) tumor cell line AU565 and target tumor cell line TYK-nu (Mut+HLA+) by T cells expressing TCR001+/−mbIL15. NT=non-transposed; TCR001 only=BPA-N, TCR001 with mbIL15=AP15 TB-NU or BP15TA-NU.

FIG. 32A-32B shows the specific lysis of a tumor cell line by T cells expressing (FIG. 32A) TCR022+/−mbIL15 or (FIG. 32B) TCR075+/−mbIL15. Tumor cell line was transfected with the appropriate HLA-expression plasmid and pulsed with either wild type (WT) or mutant (Mut) peptides and co-cultured with T cells. NT=non-transposed; TCR only=BPA-N, TCR with mbIL15=AP15 TB-NU or BP15TA-NU.

FIG. 33 shows TCR+ population for cells transposed with various TCRs+/−mbIL15 (Groups 3.1-3.30) after long-term cytokine withdrawal (LTWD). TCR only=BPA-N, TCR with mbIL15=AP15 TB-NU or BP15TA-NU.

FIG. 34A-34C shows cell survival for cells transposed with various TCRs+/−mbIL15 (Groups 3.1-3.30) after long-term cytokine withdrawal (LTWD). BPA-N (IL2)=TCR only cultured with IL2, NT=non-transposed, TCR with mbIL15=AP15 TB-NU or BP15TA-NU.

FIG. 35A-35C shows specific induction of activation marker, 4-1BB, after overnight co-culture of cells transposed with various TCRs+/−mbIL15 (Groups 3.1-3.30) after long-term cytokine withdrawal (LTWD) with wild-type or mutant neoantigen pulsed dendritic cells. BPA-N (IL2)=TCR only cultured with IL2, NT=non-transposed, TCR with mbIL15=AP15 TB-NU or BP15TA-NU.

FIG. 36A-36C shows IFN-γ secretion after overnight co-culture of cells transposed with various TCRs+/−mbIL15 (Groups 3.1-3.30) after long-term cytokine withdrawal (LTWD) with wild-type or mutant neoantigen pulsed dendritic cells. BPA-N (IL2)=TCR only cultured with IL2, NT=non-transposed, TCR with mbIL15=AP15 TB-NU or BP15TA-NU.

FIG. 37A-37C shows a comparison of 4-1BB induction in cells transposed with various TCRs+mbIL15 (Groups 3.1-3.30) pre- and post-LTWD culture after overnight co-culture with wild-type or mutant neoantigen pulsed dendritic cells.

FIG. 38 is a set of representative pie charts showing the mean frequency of live CD3⁺ T cell memory and effector subsets at day 11 post-expansion of cells transposed with TCR001 expressed from either BPA-N or with mbIL15 from either AP15 TB-NU or BP15TA-NU.

FIG. 39 is a set of representative pie charts showing the mean frequency of live CD3⁺ T cell memory and effector subsets at day 22 post-expansion of cells transposed with TCR001 expressed from either BPA-N or with mbIL15 from either AP15 TB-NU or BP15TA-NU.

FIG. 40A-40E is a set of pie charts showing the mean frequency of live CD3⁺ T cell memory and effector subsets of in cells transposed with the tested plasmids (Groups 3.1-3.30) after 4 weeks of LTWD culture.

### DETAILED DESCRIPTION

Improved methods for culturing and/or expanding lymphocytes using unconventional culture components are provided. In some embodiments, the lymphocytes are T cells. In some embodiments, the T cells have been subject to electroporation. In some embodiments, the electroporation occurs within 12 hours of contacting the T cells with a first culture medium. In some embodiments, the culture components of the first culture medium comprise one or more cytokines. In some embodiments, the one or more cytokines are selected from the group consisting of IL-7, IL-15, and IL-21. In some embodiments, the culture components of the first culture medium comprise IL-15. In some embodiments, the culture components of the first culture medium comprise an exogenous glutathione precursor. In some embodiments, the culture components of the first culture medium comprise IL-15 and an exogenous glutathione precursor. In some embodiments, the culture components of the first culture medium also comprise IL-7. In some embodiments, the culture components of the first culture medium also comprise IL-21, In some embodiments, the exogenous glutathione precursor is N-acetylcysteine (NAC). In some embodiments, the culture components of the first culture medium comprise IL-7, IL-15 and NAC.

In another aspect, the methods for culturing and/or expanding lymphocytes using unconventional culture components comprises a second culture medium. In some embodiments, the lymphocytes are T cells. In some embodiments, the T cells are cultured and/or expanded in the second culture medium after being cultured and/or expanded in the first culture medium. In some embodiments, the T cells are electroporated in the second culture medium. In some embodiments, the second culture medium comprises IL-2, IL-7, IL-12, IL-15 and/or IL-21. In some embodiments, the second culture medium comprises IL-7, IL-12 and/or IL-21. In some embodiments, the second culture medium comprises IL-7. In some embodiments, the second culture medium comprises IL-12. In some embodiments, the second culture medium comprises IL-21. In some embodiments, the second culture medium comprises IL-7 and IL-21. In some embodiments, the second culture medium comprises IL-7, IL-12 and IL-21. In some embodiments, one or more of the cytokines is provided in the second culture medium only at day 1, whereas other cytokines are replenished throughout the culture period. In some embodiments, one or more of IL-7, IL-12 and/or IL-21 are provided only on day 1 of culture. In some embodiments, IL-7 is provided only on day 1 of culture. In some embodiments, IL-12 is provided only on day 1 of culture. In some embodiments, IL-7 and IL-12 is provided only on day 1 of culture. In some embodiments, IL-21 is provided at regular intervals throughout the culture period. In some embodiments, IL-7 and IL-12 is provided only on day 1 of culture and IL-21 is provided at regular intervals throughout the culture period.

In another aspect, the methods for culturing and/or expanding lymphocytes using unconventional culture components comprise a third culture medium. In some embodiments, the lymphocytes are T cells. In some embodiments, the T cells are cultured and/or expanded in the third culture medium after being cultured and/or expanded in the first culture medium. In some embodiments, the T cells are cultured and/or expanded in the third culture medium after being cultured and/or expanded in the second culture medium. In some embodiments, the T cells are cultured and/or expanded in the third culture medium after being cultured and/or expanded in the first and second culture media. In some embodiments, the third culture medium comprises one or more of IL-2, IL-12, IL-15 and IL-21. In some embodiments, the third culture medium comprises one or more of IL-2, IL-12 and IL-21. In some embodiments, the third culture medium comprises IL-2. In some embodiments, the third culture medium comprises IL-12. In some embodiments, the third culture medium comprises IL-21. In some embodiments, the third culture medium comprises NAC. In some embodiments, the third culture medium comprises IL-12, IL-21 and NAC. In some embodiments, the third culture medium comprises IL-2, IL-12, IL-21 and NAC. In some embodiments, one or more of IL-2, IL-12 and/or IL-21 are provided only on day 1 of culture. In some embodiments, IL-12 is provided only on day 1 of culture. In some embodiments, IL-2 and IL-21 are provided at regular intervals throughout the culture period. In some embodiments, IL-12 is provided only on day 1 of culture and IL-2 and IL-21 are provided at regular intervals throughout the culture period.

In certain embodiments, the first culture medium utilized in the methods herein does not comprise IL-2, IL-12, or IL-21; both IL-2 and IL-21; both IL-2 and IL-12; both IL-12 and IL-21; or all of IL-2, IL-12 and IL-21. In certain embodiments, the second culture medium does not comprise IL-2 or IL-15; both IL-2 and IL-15. In certain embodiments, the third culture medium does not comprise IL-2, IL-7 or IL-15; both IL-2 and IL-15; both IL-2 and IL-7; both IL-7 or IL15; or all of IL-2, IL-7 and IL-15.

In some embodiments, the culture media described herein also include one or more TCR agonists and one or more agonists of a T cell costimulatory molecule. In some embodiments, the TCR agonist is a CD3 agonist. In some embodiments, the agonist of a T cell costimulatory molecule is a CD28 agonist. In some embodiments, the culture media described herein also include a nanomatrix. In some embodiments, the TCR agonist and/or the T cell costimulatory molecule is associated with the nanomatrix. In other embodiments, the T cells are cultured with feeder cells.

In some embodiments, the first, second and/or third, utilized in the described methods is supplemented with one or more T cell-stimulating cytokines at a time interval selected from the group consisting of 1 day, 2 days, 3 days, 4 days, 5 days, and 6 days. In some embodiments, the T cell-stimulating cytokine that is supplemented is IL-2 and/or IL-21. In one embodiment, 30% to 99% of the first culture medium is changed at a time interval selected from the group consisting of 1 day, 2 days, 3 days, 4 days, 5 days, and 6 days.

In another aspect, the present disclosure provides a population of engineered T cells, wherein most of the engineered T cells in the population comprise an exogenous TCR or functional fragment thereof, and wherein more than 20% of the population of engineered T cells are CCR7+/CD45RO+. In another aspect, the present disclosure provides a population of engineered T cells, and wherein most of the engineered T cells in the population comprise an exogenous TCR or functional fragment thereof, wherein more than 20% of the population of engineered T cells are memory T cells (e.g., a central memory T cell, an effector memory T cell, a stem cell-like memory T cells). In some embodiments, the T cells are electroporated with a vector that expresses the exogenous TCR or functional fragment thereof. In some embodiments, the T cells are cultured and/or expanded according to any of the methods provided herein.

In another aspect, the present disclosure provides a population of engineered T cells, wherein most of the engineered T cells in the population comprise an exogenous TCR or functional fragment thereof, and wherein more than 40% of the population of engineered T cells are CD95+/CD62L+. In another aspect, the present disclosure provides a population of engineered T cells, wherein most of the engineered T cells in the population comprise an exogenous TCR or functional fragment thereof, and wherein more than 20% of the population of engineered T cells are memory T cells (e.g., a central memory T cell, an effector memory T cell, a stem cell memory T cells). In some embodiments, the T cells are electroporated with a vector that expresses the exogenous TCR or functional fragment thereof. In some embodiments, the T cells are cultured and/or expanded according to any of the methods provided herein.

### 1.1 Definitions

Generally, nomenclature used in connection with cell and tissue culture, molecular biology, immunology, microbiology, genetics, and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of molecular and cell biology and biochemistry described herein are well-known and commonly used in the art.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

As used herein, the terms “about” and “approximately,” when used to modify a numeric value or numeric range, indicate that deviations of 5% to 10% above (e.g., up to 5% to 10% above) and 5% to 10% below (e.g., up to 5% to 10% below) the value or range remain within the intended meaning of the recited value or range.

As used herein, the phrase “lymphocytes” refers to B cells, T cells or natural killer (NK) cells. T cells include, but are not limited to, naïve T cells (CD4+ or CD8+); killer CD8+ T cells; cytotoxic CD4+ T cells; helper CD4+ T cells; CD4+ T cells corresponding to Th1, Th2, Th9, Th17, Th22, follicular helper (Tfh), regulatory (Treg) lineages; tumor infiltrating lymphocytes (TILs); and memory T cells (central memory, effector memory, stem cell memory, stem cell-like memory).

As used herein, the phrase “electroporated lymphocytes” or “electroporated T cells” refers to a population of lymphocytes or T cells that have been electroporated with a one or more exogenous nucleic acids (e.g., a plasmid).

As used herein, the phrase “population of cells” refers to a number of cells, e.g., electroporated T cells that share common traits. In general, populations generally range from 1×10⁶ to 1×10¹¹ in number, with different T cell populations comprising different numbers. In some embodiments, the population of cells is monoclonal. In other embodiments, the population of cells is polyclonal. In some embodiments, when the population of cells is polyclonal, the cells still share one or more common traits. A monoclonal T cell population will result in the predominance of a single TCR-gene rearrangement pattern. In contrast, polyclonal T cell populations have diverse TCR-gene rearrangement pattern, which can make them more effective in certain situations. As used herein, the phrase “expanding a population of cells” is synonymous with “proliferating a population of cells” and refers to increasing the number of cells in an electroporated population.

As used herein, the phrase “expansion process” refers to the process whereby the number of cells in an electroporated T cell population is increased. Processes where electroporated T cells are merely isolated or enriched without substantial increase in the number of electroporated T cells are not expansion processes.

As used herein, “exogenous glutathione precursor” refers to compounds that increase the levels of glutathione in a cell culture media. Exogenous glutathione precursors include, cysteine, glycine, glutamate, glutamine, N-acetylcysteine (NAC), and N-acetylcysteine amide (NACA). In some embodiments, the exogenous glutathione precursor is NAC.

As used herein, the term “cytokine” refers to a broad category of small proteins (about 5-20 kDa in size) that are important in cell signaling. Cytokines are peptides and cannot cross the lipid bilayer of cells to enter the cytoplasm. Cytokines have been shown to be involved in autocrine signaling, paracrine signaling, and endocrine signaling as immunomodulating agents. Cytokines include chemokines, interferons, interleukins, lymphokines, and tumor necrosis factors, but generally not hormones or growth factors, although there is some overlap in terminology. Cytokines are produced by a broad range of cells, including immune cells like macrophages, B lymphocytes, T lymphocytes, and mast cells, as well as endothelial cells, fibroblasts, and various stromal cells. Cytokines generally act through binding to cell-surface receptors and are especially important in the immune response, since they are involved in regulating the maturation, growth, and responsiveness of particular cell populations.

As used herein, the phrase “T cell-stimulating cytokine” refers to a cytokine that stimulates and/or activates T cell lymphocytes. In some embodiments, the T-cell stimulating cytokine is IL-2, IL-7, IL-12, IL-15 or IL-21. In certain embodiments, T cell-stimulating cytokines are produced in a cell from a viral vector.

As used herein, the term “IL-2” (also referred to herein as “IL2”) refers to the cytokine and T cell growth factor known as interleukin-2, and includes all forms of IL-2, including human and mammalian forms, forms with conservative amino acid substitutions, glycoforms, biosimilars, and variants thereof. IL-2 is described, e.g., in Nelson, J. Immunol. 2004, 172, 398388 and Malek, Annu. Rev. Immunol. 2008, 26, 453-79, the disclosures of which are incorporated herein by reference in their entireties. The term IL-2 encompasses human, recombinant forms of IL-2, such as aldesleukin (PROLEUKIN, available commercially from multiple suppliers in 22 million IU per single use vials), as well as the form of recombinant IL-2 commercially supplied by CellGenix, Inc., Portsmouth, N.H., USA (CELLGRO GMP) or ProSpec-Tany TechnoGene Ltd., East Brunswick, N.J., USA (Cat. No. CYT-209-b) and other commercial equivalents from other vendors. Aldesleukin (des-alanyl-1, serine-125 human IL-2) is a nonglycosylated human recombinant form of IL-2 with a molecular weight of approximately 15 kDa. The term IL-2 also encompasses pegylated forms of IL-2, including the pegylated IL-2 prodrug NKTR-214, available from Nektar Therapeutics, South San Francisco, Calif., USA. NKTR-214 and pegylated IL-2 suitable for use in the invention is described in U.S. Patent Application Publication No. US 2014/0328791 A1 and International Patent Application Publication No. WO 2012/065086 A1, the disclosures of which are incorporated herein by reference in their entireties. Alternative forms of conjugated IL-2 suitable for use in the invention are described in U.S. Pat. Nos. 4,766,106, 5,206,344, 5,089,261 and 4,902,502, the disclosures of which are incorporated herein by reference in their entireties. Formulations of IL-2 suitable for use in the invention are described in U.S. Pat. No. 6,706,289, the disclosure of which is incorporated herein by reference in its entirety. The human IL2 gene is identified by NCBI Gene ID 3558. An exemplary nucleotide sequence for a human IL2 gene is the NCBI Reference Sequence: NG 016779.1.

Interleukin-2 (IL-2) is an interleukin, a type of cytokine signaling molecule in the immune system. It is a 15.5-16 kDa protein that regulates the activities of white blood cells (leukocytes, often lymphocytes) that are responsible for immunity. IL-2 is part of the body's natural response to microbial infection. IL-2 mediates its effects by binding to IL-2 receptors, which are expressed by lymphocytes. The major sources of IL-2 are activated CD4+ T cells and activated CD8+ T cells.

IL-2 has essential roles in key functions of the immune system, tolerance and immunity, primarily via its direct effects on T cells. In the thymus, where T cells mature, it prevents autoimmune diseases by promoting the differentiation of certain immature T cells into regulatory T cells, which suppress other T cells that are otherwise primed to attack normal healthy cells in the body. IL-2 enhances activation-induced cell death (AICD). IL-2 also promotes the differentiation of T cells into effector T cells and into memory T cells when the initial T cell is also stimulated by an antigen, thus helping the body fight off infections. Together with other polarizing cytokines, IL-2 stimulates naive CD4+ T cell differentiation into Th1 and Th2 lymphocytes while it impedes differentiation into Th17 and follicular helper T (Tfh) lymphocytes. Its expression and secretion are tightly regulated and functions as part of both transient positive and negative feedback loops in mounting and dampening immune responses. Through its role in the development of T cell immunologic memory, which depends upon the expansion of the number and function of antigen-selected T cell clones, it plays a role in enduring cell-mediated immunity.

The methods for expanding populations of electroporated T cells as provided in the present disclosure utilize IL-15. IL-15 (also referred to herein as “IL15”) refers to the cytokine and T cell growth factor known as interleukin-15, and as utilized in the present invention, includes all forms of IL-15, including human and other mammalian forms, forms with conservative amino acid substitutions, glycoforms, biosimilars, and variants thereof. IL-15 is described, e.g., in Steel J C, Waldmann T A, Morris J C (January 2012) “Interleukin-15 biology and its therapeutic implications in cancer,” Trends in Pharmacological Sciences, 33 (1): 35-41 and Waldmann T A, Tagaya Y (1999) “The multifaceted regulation of interleukin-15 expression and the role of this cytokine in NK cell differentiation and host response to intracellular pathogens,” Annual Review of Immunology, 17: 19-49, the disclosures of which are incorporated herein by reference in their entireties. The term IL-15 also encompasses recombinant forms of IL-15. As used herein, the term IL-15 also encompasses pegylated forms of IL-15. The human IL15 gene is identified by NCBI Gene ID 3600. An example nucleotide sequence for a human IL15 gene is the NCBI Reference Sequence: NG 029605.2.

IL-7 is a cytokine secreted by stromal cells in the bone marrow and thymus. It is also produced by keratinocytes, dendritic cells, hepatocytes, neurons, and epithelial cells, but is not produced by normal lymphocytes. IL-7 stimulates the differentiation of multipotent (pluripotent) hematopoietic stem cells into lymphoid progenitor cells (as opposed to myeloid progenitor cells where differentiation is stimulated by IL-3). It also stimulates proliferation of all cells in the lymphoid lineage (B cells, T cells and NK cells). It is important for proliferation during certain stages of B-cell maturation, T and NK cell survival, development and homeostasis. An example nucleotide sequence for a human IL7 gene is the NCBI Reference Sequence: AH006906.2.

IL-21 is a cytokine that has potent regulatory effects on cells of the immune system, including natural killer (NK) cells and cytotoxic T cells that can destroy virally infected or cancerous cells. This cytokine induces cell division/proliferation in its target cells. IL-21 is expressed in activated human CD4+ T cells but not in most other tissues. In addition, IL-21 expression is up-regulated in Th2 and Th17 subsets of T helper cells, as well as follicular helper T cells. In fact, it was shown that IL-21 can be used to identify peripheral follicular helper T cells. Furthermore, IL-21 is expressed in NK T cells regulating the function of these cells. An example nucleotide sequence for a human IL21 gene is the NCBI Reference Sequence: LC133256.1.

IL-12 is a cytokine that is naturally produced by dendritic cells, macrophages, neutrophils, and human B-lymphoblastoid cells (NC-37) in response to antigenic stimulation. This cytokine is involved in the differentiation of naive T cells into Th1 cells. IL-12 also plays an important role in the activities of natural killer cells and T lymphocytes. Moreover, IL-12 mediates enhancement of the cytotoxic activity of NK cells and CD8+ cytotoxic T lymphocytes. An example nucleotide sequence for a human IL12 gene is the NCBI Reference Sequence: NM000882.4.

In some embodiments, the T cell-stimulating cytokine(s) utilized in the methods herein is selected from the group consisting of IL-2, IL-7, IL-12, IL-15, IL-21, and combinations thereof. In some embodiments, the final concentration of the T cell-stimulating cytokine(s) utilized in the culture media described herein is from about 10 U/ml to about 7,000 U/ml. In some embodiments, the final concentration of T cell-stimulating cytokine(s) utilized in the first culture medium is from about 5 ng/ml to about 3,500 ng/ml.

As used herein, the term “medium” refers to a liquid or gel designed to support the survival, growth, and/or proliferation of cells in an artificial environment. A medium generally comprises a defined set of components. Such components may include an energy source, growth factors, hormones, stimulants, activators, sugars, salts, vitamins, and/or amino acids, and/or a combination of these. In many embodiments, the medium is cell culture medium.

As used herein, the phrase “components of the medium are maintained” refers to a medium comprising a defined set of components, such as particular stimulants and activators, where the identity of the components remains constant, but the concentration of one or more of the components may be varied. In certain embodiments, the concentration of one or more components in the media varies over time while the cells are cultured in the media. However, when the media is changed the fresh media has the same components for each change. In some embodiments, in order to maintain the components of a medium the medium must be changed at a certain interval. In some embodiments, the medium is changed every, 2, 4, 8, or 12 hours. In some embodiments, the medium is changed every, 1, 2, 3, 4, 5, 6, 7, 8, 9, 10, 11, 12, 13 or 14 days.

As used herein, the phrase “anti-CD3 antibody” refers to an antibody or variant thereof, e.g., a monoclonal antibody, and includes human, humanized, chimeric or murine antibodies which are directed against the CD3 receptor in the T cell antigen receptor of mature T cells. Anti-CD3 antibodies include OKT-3, also known as muromonab. Anti-CD3 antibodies also include the UCHT1 clone, also known as T3 and CD3c. Other anti-CD3 antibodies include, for example, otelixizumab, teplizumab, and visilizumab.

As used herein, the phrase “anti-CD28 antibody” refers to an antibody or variant thereof, e.g., a monoclonal antibody, and includes human, humanized, chimeric or murine antibodies which are directed against the CD28 receptor in the T cell antigen receptor of mature T cells.

In some embodiments, an anti-4-1BB antibody can be utilized as a 4-1BB ligand.

As used herein, the phrase “anti-4-1BB antibody” refers to an antibody or variant thereof, e.g., a monoclonal antibody, and includes human, humanized, chimeric or murine antibodies which are directed against 4-1BB.

As used herein, the phrase “anti-CD2 antibody” refers to an antibody or variant thereof, e.g., a monoclonal antibody, and includes human, humanized, chimeric or murine antibodies which are directed against the CD2 receptor in the T cell antigen receptor of mature T cells.

As used herein, the term “OKT-3” (also referred to herein as “OKT3”) refers to the anti-CD3 antibody produced by Miltenyi Biotech, Inc., San Diego, Calif., USA) and or biosimilar or variant thereof (e.g., a humanized, chimeric, or affinity matured variant). A hybridoma capable of producing OKT-3 is available in the American Type Culture Collection and assigned the ATCC accession number CRL 8001. A hybridoma capable of producing OKT-3 is available in the European Collection of Authenticated Cell Cultures (ECACC) and assigned Catalogue No. 86022706.

As used herein, the term “UCHT1” refers to the anti-CD3 antibody described in Beverley and Callard (1981) Eur. J. Immunol. 11: 329-334, and or biosimilar or variant thereof (e.g., a humanized, chimeric, or affinity matured variant). A hybridoma capable of producing an exemplary UCHT1 is available from Creative Diagnostics, Shirley, NY, USA, and assigned Catalogue No. CSC-H3068.

As used herein, the phrase “activation signal” refers to one or more non-endogenous stimuli that cause T cells to become activated. In the endogenous process, T cells become activated when they are presented with peptide antigens by MHC class II molecules, which are expressed on the surface of antigen-presenting cells (APCs). Once activated, the T cells divide rapidly and secrete cytokines that regulate or assist the immune response. The endogenous T cell activation process involves at least (a) activation of the TCR complex, which involves CD3, and (b) co-stimulation of CD28 or 4-1BB by proteins on the APC surface. It is known in the art that the endogenous activation of T cells can be simulated by stimulation of T cells by CD3, CD28 or 4-1BB agonists (e.g., antibodies). Thus, CD3, CD28 and/or 4-1BB can together provide an activation signal to T cells.

As used herein, the phrase “feeder cell” refers to cells used to provide extracellular secretions that help another cell type proliferate. In certain embodiments, the feeder cells referred to herein are peripheral blood mononuclear cell (PBMC) or an antigen-presenting cell (APC).

As used herein, the term “nanomatrix” refers to a colloidal suspension of more than one matrix of polymer chains. A nanomatrix is a multiphase material that has dimensions of less than 500 nm or structures having nanoscale repeat distances between the different phases that make up the material. Polymers may include polyethylene, polypropylene, polystyrene, polysaccharide, dextran, and other macromolecules, which are composed of many repeated subunits. A nanomatrix may also have embedded additional functional compounds, such as magnetic, paramagnetic, or superparamagnetic nanocrystals. In addition, functional moieties, such as ligands or agonists can be covalently attached or bound to the polymer chains for specific applications.

As used herein, the term “matrix” or “mobile matrix” refers to a discrete, isolatable, three-dimensional lattice-type structure where the backbone of the structure can be flexible or mobile and can be composed of materials, such as polymers and ceramics. Being a three-dimensional structure, a matrix can have a smallest dimension and a largest dimension, such as a length. A mobile matrix may be of collagen, purified proteins, purified peptides, polysaccharides, glycosaminoglycans, or extracellular matrix compositions. A polysaccharide may include for example, cellulose ethers, starch, gum arabic, agarose, dextran, chitosan, hyaluronic acid, pectins, xanthan, guar gum, or alginate. Other polymers may include polyesters, polyethers, polyacrylates, polyacrylamides, polyamines, polyethylene imines, polyquaternium polymers, polyphosphazenes, polyvinylalcohols, poly vinylacetates, polyvinylpyrrolidones, block copolymers, or polyurethanes. The mobile matrix may comprise a polymer of dextran. “Matrices” refers to a collection of more than one matrix.

As used herein, the phrase “largest dimension” in the context of a matrix refers to the longest length of the matrix.

As used herein, the term “dextran” refers to a complex branched glucan, a polysaccharide derived from the condensation of glucose. Dextran chains are of varying lengths, from 3 to 2000 kilodaltons. The polymer main chain consists of α-1,6 glycosidic linkages between glucose monomers, with branches from α-1,3 linkages.

As used herein, the phrase “agonists bound to a nanomatrix” refers to agonists that are covalently attached to the polymer chains that comprise the matrices within the nanomatrix.

As used herein, the phrase “colloidal suspension” refers to a mixture in which one substance, such as a matrix, is suspended throughout another substance, such as a liquid. A colloidal suspension thus has a dispersed phase, i.e., the suspended substance, and a continuous phase, i.e., the medium of suspension, such as a liquid.

As used herein, the phrase “contacting a population of T cells with a nanomatrix” refers to bringing a population of T cells and the nanomatrix together such that the population of T cells can associate with nanomatrix-bound functional moieties, such as ligands or agonists, or nanomatrix-embedded functional compounds through ionic, hydrogen-bonding, or other types of physical or chemical interactions.

As use herein, the phrase “colloidal polymer chains” refers to polymer chains that when linked to each other through covalent bonds or other physical or chemical interactions can form colloidal suspensions.

As used herein, the terms “T cell receptor” and “TCR” are used interchangeably and refer to molecules comprising CDRs or variable regions from αβ T cell receptors. Examples of TCRs include, but are not limited to, full-length TCRs, antigen-binding fragments of TCRs, soluble TCRs lacking transmembrane and cytoplasmic regions, single-chain TCRs containing variable regions of TCRs attached by a flexible linker, TCR chains linked by an engineered disulfide bond, single TCR variable domains, single peptide-MHC-specific TCRs, multi-specific TCRs (including bispecific TCRs), TCR fusions, TCRs comprising co-stimulatory regions, human TCRs, humanized TCRs, chimeric TCRs, recombinantly produced TCRs, and synthetic TCRs. In certain embodiments, the TCR is a full-length TCR comprising a full-length α chain and a full-length β chain. In certain embodiments, the TCR is a soluble TCR lacking transmembrane and/or cytoplasmic region(s). In certain embodiments, the TCR is a single-chain TCR (scTCR) comprising Vα and Vβ linked by a peptide linker, such as a scTCR having a structure as described in PCT Publication No.: WO 2003/020763, WO 2004/033685, or WO 2011/044186, each of which is incorporated by reference herein in its entirety. In certain embodiments, the TCR comprises a transmembrane region. In certain embodiments, the TCR comprises a co-stimulatory signaling region.

As used herein, the term “full-length TCR” refers to a TCR comprising a dimer of a first and a second polypeptide chain, each of which comprises a TCR variable region and a TCR constant region comprising a TCR transmembrane region and a TCR cytoplasmic region. In certain embodiments, the full-length TCR comprises one or two unmodified TCR chains, e.g., unmodified α or βTCR chains. In certain embodiments, the full-length TCR comprises one or two altered TCR chains, such as chimeric TCR chains and/or TCR chains comprising one or more amino acid substitutions, insertions, or deletions relative to an unmodified TCR chain. In certain embodiments, the full-length TCR comprises a mature, full-length TCR α chain and a mature, full-length TCR β chain.

As used herein, the term “TCR variable region” refers to the portion of a mature TCR polypeptide chain (e.g., a TCR α chain or β chain) which is not encoded by the TRAC gene for TCR α chains, either the TRBC1 or TRBC2 genes for TCR β chains, or the TRDC gene for TCR δ chains. In some embodiments, the TCR variable region of a TCR α chain encompasses all amino acids of a mature TCR α chain polypeptide which are encoded by a TRAV and/or TRAJ gene, and the TCR variable region of a TCR β chain encompasses all amino acids of a mature TCR β chain polypeptide which are encoded by a TRBV, TRBD, and/or TRBJ gene (see, e.g., Lefranc and Lefranc, (2001) “T cell receptor FactsBook.” Academic Press, ISBN 0-12-441352-8, which is incorporated by reference herein in its entirety). TCR variable regions generally comprise framework regions (FR) 1, 2, 3, and 4 and complementarity determining regions (CDR) 1, 2, and 3.

As used herein, the terms “a chain variable region” and “Vα” are used interchangeably and refer to the variable region of a TCR α chain.

As used herein, the terms “β chain variable region” and “Vβ” are used interchangeably and refer to the variable region of a TCR β chain.

As used herein in the context of a TCR, the term “CDR” or “complementarity determining region” means the noncontiguous antigen combining sites found within the variable regions of a TCR chain (e.g., an α chain or a β chain). These regions have been described in Lefranc, (1999) The Immunologist 7: 132-136; Lefranc et al., (1999) Nucleic Acids Res 27: 209-212; Lefranc (2001) “T cell receptor FactsBook.” Academic Press, ISBN 0-12-441352-8; Lefranc et al., (2003) Dev Comp Immunol. 27(1):55-77; and in Kabat et al., (1991) “Sequences of protein of immunological interest,” each of which is herein incorporated by reference in its entirety. In certain embodiments, CDRs are determined according to the IMGT numbering system described in Lefranc (1999) supra. In certain embodiments, CDRs are defined according to the Kabat numbering system described in Kabat supra. In certain embodiments, CDRs are defined empirically, e.g., based upon a structural analysis of the interaction of a TCR with a cognate antigen (e.g., a peptide or a peptide-MHC complex). In certain embodiments, the α chain and β chain CDRs of a TCR are defined according to different conventions (e.g., according to the Kabat or IMGT numbering systems, or empirically based upon structural analysis).

As used herein, the term “framework amino acid residues” refers to those amino acids in the framework region of a TCR chain (e.g., an α chain or a β chain). The term “framework region” or “FR” as used herein includes the amino acid residues that are part of the TCR variable region, but are not part of the CDRs.

As used herein, the term “constant region” with respect to a TCR refers to the portion of a TCR that is encoded by the TRAC gene (for TCR α chains) or either the TRBC1 or TRBC2 gene (for TCR β chains), optionally lacking all or a portion of a transmembrane region and/or all or a portion of a cytoplasmic region. In certain embodiments, a TCR constant region lacks a transmembrane region and a cytoplasmic region. A TCR constant region does not include amino acids encoded by a TRAV, TRAJ, TRBV, TRBD, TRBJ, TRDV, TRDD, TRDJ, TRGV, or TRGJ gene (see, e.g., “T cell receptor FactsBook,” supra).

As used herein, the terms “major histocompatibility complex” and “MHC” are used interchangeably and refer to an MHC class I molecule and/or an MHC class II molecule.

As used herein, the term “MHC class I” refers to a dimer of an MHC class I α chain and a β2 microglobulin chain and the term “MHC class II” refers to a dimer of an MHC class II α chain and an MHC class II β chain.

As used herein, the terms “human leukocyte antigen” and “HLA” are used interchangeably and can also refer to the proteins encoded by the MHC genes. HLA-A, HLA-B, HLA-C, HLA-E, HLA-F, and HLA-G refer to major and minor gene products of MHC class I genes. HLA-DP, HLA-DQ, and HLA-DR refer to gene products of MHC class I genes, which are expressed on antigen-presenting cells, B cells, and T cells.

As used herein, the term “peptide-MHC complex” refers to an MHC molecule (MHC class I or MHC class II) with a peptide bound in the art-recognized peptide binding pocket of the MHC. In some embodiments, the MHC molecule is a membrane-bound protein expressed on the cell surface. In some embodiments, the MHC molecule is a soluble protein lacking transmembrane or cytoplasmic regions.

As used herein, the term “extracellular” with respect to a recombinant transmembrane protein refers to the portion or portions of the recombinant transmembrane protein that are located outside of a cell.

As used herein, the term “transmembrane” with respect to a recombinant transmembrane protein refers to the portion or portions of the recombinant transmembrane protein that are embedded in the plasma membrane of a cell.

As used herein, the term “cytoplasmic” with respect to a recombinant transmembrane protein refers to the portion or portions of the recombinant transmembrane protein that are located in the cytoplasm of a cell.

As used herein, the term “co-stimulatory signaling region” refers to the intracellular portion of a co-stimulatory molecule that is responsible for mediating intracellular signaling events.

“Binding affinity” generally refers to the strength of the sum total of non-covalent interactions between a single binding site of a molecule (e.g., a TCR) and its binding partner (e.g., a peptide-MHC complex). Unless indicated otherwise, as used herein, “binding affinity” refers to intrinsic binding affinity which reflects a 1:1 interaction between members of a binding pair (e.g., a TCR and a peptide-MHC complex). The affinity of a molecule X for its partner Y can generally be represented by the dissociation constant (K_{D}). Affinity can be measured and/or expressed in a number of ways known in the art, including, but not limited to, equilibrium dissociation constant (K_{D}) and equilibrium association constant (K_{A}). The K_{D} is calculated from the quotient of k_{off}/kₒₙ, whereas K_{A} is calculated from the quotient of kₒₙ/k_{off}. Kₒₙ refers to the association rate constant and k_{off} refers to the dissociation rate constant. The kₒₙ and k_{off} can be determined by techniques known to one of ordinary skill in the art, such as use of BIAcore® or KinExA. As used herein, a “lower affinity” refers to a larger K_{D}.

“Avidity” generally refers to the affinity of a binding molecule (e.g., a TCR) and its binding partner (e.g., a peptide-MHC complex). Binding molecules described herein are able to bind antigen via two (or more) sites in which the multiple interactions synergize to enhance the “apparent” affinity. Avidity is the measure of the strength of binding between the binding molecule described herein (e.g., a TCR) and the pertinent antigens (e.g., a peptide-MHC complex). Avidity is related to both the affinity between an antigenic determinant and its antigen binding site on the antigen-binding molecule and the number of pertinent binding sites present on the antigen-binding molecules.

For example, “specifically binds to” may be used to refer to the ability of a TCR to preferentially bind to a particular antigen (e.g., a specific peptide or a specific peptide-MHC complex combination) as such binding is understood by one skilled in the art. For example, a TCR that specifically binds to an antigen can bind to other antigens, generally with lower affinity as determined by, e.g., BIAcore®, or other immunoassays known in the art (see, e.g., Savage et al., (1999) Immunity. 10(4):485-92, which is incorporated by reference herein in its entirety). In a specific embodiment, a TCR that specifically binds to an antigen binds to the antigen with an association constant (Kₐ) that is at least 2-fold, 5-fold, 10-fold, 50-fold, 100-fold, 500-fold, 1,000-fold, 5,000-fold, or 10,000-fold greater than the Kₐ when the TCR binds to another antigen.

As used herein, an “epitope” is a term in the art and refers to a localized region of an antigen (e.g., a peptide or a peptide-MHC complex) to which a TCR can bind. In certain embodiments, the epitope to which a TCR binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), flow cytometry analysis, mutagenesis mapping (e.g., site-directed mutagenesis mapping), and/or structural modeling. For X-ray crystallography, crystallization may be accomplished using any of the known methods in the art (e.g., Giegé R et al., (1994) Acta Crystallogr D Biol Crystallogr 50(Pt 4): 339-350; McPherson A, (1990) Eur J Biochem 189: 1-23; Chayen NE, (1997) Structure 5: 1269-1274; McPherson A, (1976) J Biol Chem 251: 6300-6303, each of which is herein incorporated by reference in its entirety). TCR:antigen crystals may be studied using well-known X-ray diffraction techniques and may be refined using computer software such as X-PLOR (Yale University, 1992, distributed by Molecular Simulations, Inc.; see, e.g., Meth Enzymol (1985) volumes 114 & 115, eds Wyckoff H. W., et al.; U.S. 2004/0014194); and BUSTER (Bricogne G, (1993) Acta Crystallogr D Biol Crystallogr 49(Pt 1): 37-60; Bricogne G, (1997) Meth Enzymol 276A: 361-423, ed Carter C W; and Roversi P et al., (2000) Acta Crystallogr D Biol Crystallogr 56(Pt 10): 1316-1323), each of which is herein incorporated by reference in its entirety. Mutagenesis mapping studies may be accomplished using any method known to one of skill in the art. See, e.g., Champe M et al., (1995) J Biol Chem 270: 1388-1394 and Cunningham B C & Wells J A, (1989) Science 244: 1081-1085, each of which is herein incorporated by reference in its entirety, for a description of mutagenesis techniques, including alanine scanning mutagenesis techniques. In a specific embodiment, the epitope of an antigen is determined using alanine scanning mutagenesis studies. In a specific embodiment, the epitope of an antigen is determined using hydrogen/deuterium exchange coupled with mass spectrometry. In certain embodiments, the antigen is a peptide-MHC complex. In certain embodiments, the antigen is a peptide presented by an MHC molecule.

As used herein, the terms “treat,” “treating,” and “treatment” refer to therapeutic or preventative measures described herein. In some embodiments, the methods of “treatment” employ administration of a TCR or a cell expressing a TCR to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F, (1990) PNAS 87: 2264-2268, modified as in Karlin S & Altschul S F, (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., at score=100, wordlength=12 to obtain nucleotide sequences homologous to a nucleic acid molecule described herein. BLAST protein searches can be performed with the XBLAST program parameters set, e.g., at score=50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules. Id. When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, (1988) CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the terms “antibody” and “antibodies” include full-length antibodies, antigen-binding fragments of full-length antibodies, and molecules comprising antibody CDRs, VH regions, or VL regions. Examples of antibodies include monoclonal antibodies, recombinantly produced antibodies, monospecific antibodies, multi-specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁ or IgA₂), or any subclass (e.g., IgG₂ₐ or IgG_{2b}) of immunoglobulin molecule. In certain embodiments, antibodies described herein are IgG antibodies, or a class (e.g., human IgG₁ or IgG₄) or subclass thereof. In a specific embodiment, the antibody is a humanized monoclonal antibody. In another specific embodiment, the antibody is a human monoclonal antibody.

As used herein, the term “cistron” refers to a polynucleotide sequence from which a transgene product can be produced.

As used herein, the term “polycistronic vector” refers to a polynucleotide vector that comprises a polycistronic expression cassette.

As used herein, the term “polycistronic expression cassette” refers to a polynucleotide sequence wherein the expression of three or more transgenes is regulated by common transcriptional regulatory elements (e.g., a common promoter) and can simultaneously express three or more separate proteins from the same mRNA. Exemplary polycistronic vectors, without limitation, include tricistronic vectors (containing three cistrons) and tetracistronic vectors (containing four cistrons).

As used herein, the term “polycistronic polynucleotide” refers to a polynucleotide that comprises three or more cistrons.

As used herein, the term “transcriptional regulatory element” refers to a polynucleotide sequence that mediates regulation of transcription of another polynucleotide sequence. Exemplary transcriptional regulatory elements include, but are not limited to, promoters and enhancers.

As used herein, a “furin recognition site” refers to an amino acid sequence, or a nucleotide sequence encoding the amino acid sequence, which can be cleaved by the furin enzyme. The furin enzyme is also known as PACE. In some embodiments, the furin recognition site comprises the amino acid sequence RXXR (SEQ ID NO: 1), wherein X at position 2 is any amino acid and X at position 3 is arginine or lysine. In some embodiments, the furin recognition site comprises the sequences shown below in Table 1.

TABLE 1
Amino acid sequences of exemplary furin recognition sites and polynucleotide
sequences encoding same.

| | | SEQ |
|---|---|---|
| | | ID |
| Description | Sequence | NO: |
| Furin recognition site | RAKR | 2 |
| Furin recognition site | CGGGCGAAACGC | 3 |
| polynucleotide coding | | |
| sequence | | |
| Furin recognition site | RAKRSGSG | 4 |
| (alternative) | | |
| Furin recognition site | CGGGCGAAACGCTCTGGAAGCGGA | 5 |
| (alternative) | | |
| polynucleotide coding | | |
| sequence | | |

In some embodiments, the furin recognition site comprises an amino acid sequence that is identical to the amino acid sequence of SEQ ID NO: 2 or 4, or comprises 1, 2, or 3 amino acid modifications, relative to SEQ ID NO: 2 or 4; or is encoded by a polynucleotide sequence 75%, 80%, 85%, 90%, 95%, 96%, 97%, 98%, 99%, or 100% identical to the polynucleotide sequence of SEQ ID NO: 3 or 5. In some embodiments, when positioned in a vector between a first polynucleotide sequence encoding a first protein and a second polynucleotide sequence encoding a second protein, the furin recognition site is capable of mediating the cleavage (via furin) of the first protein from the second protein, resulting in two distinct polypeptides from the same mRNA molecule.

Responsive to recognition of the furin recognition site by the furin enzyme, the furin enzyme induces cleavage of a given polypeptide on the C-terminal side of the furin recognition site or a portion thereof. Accordingly, polypeptides produced by furin-mediated cleavage at a furin recognition site may retain all or a portion of the furin recognition site on their C-terminus. For example, the C-terminus of a first polypeptide of the present disclosure may comprise the amino acid sequence RAKR (SEQ ID NO: 2) or RA.

As used herein, a “2A element” refers to a polynucleotide sequence which, when expressed in an mRNA, can induce ribosomal skipping during translation of the mRNA in a cell. Thus, two separate polypeptides may be produced from a single mRNA molecule. An amino acid sequence encoded by a 2A element is referred to as a “self-cleaving peptide.” 2A elements may be viral in origin. Exemplary 2A elements include T2A elements, P2A elements, E2A elements, and F2A elements.

As used herein, the term “P2A element” refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 19, or 21; (ii) encodes the amino acid sequence of SEQ ID NO: 18, or 20; or (iii) encodes the amino acid sequence of SEQ ID NO: 18, or 20, comprising 1, 2, or 3 amino acid modifications. In some embodiments, when positioned in a vector between a first polynucleotide sequence encoding a first protein and a second polynucleotide sequence encoding a second protein, the P2A element is capable of mediating the translation of the first polynucleotide sequence and the second polynucleotide sequence as two distinct polypeptides from the same mRNA molecule by preventing the synthesis of a peptide bond, e.g., between the penultimate residue (e.g., glycine) and the ultimate residue (e.g., proline) at the C terminus of the translation product of the P2A element, e.g., such that the penultimate residue (e.g., glycine) becomes the C-terminal residue of the first protein and the ultimate residue (e.g., proline) becomes the N-terminal residue of the second protein. In some embodiments, the P2A element additionally comprises, at its 5′ end, a polynucleotide sequence that encodes a furin recognition site, e.g., RAKR (SEQ ID NO: 2). In some embodiments, the P2A element additionally comprises, at its 5′ end, a polynucleotide sequence that encodes a furin recognition site, e.g., RAKRSGSG (SEQ ID NO: 4), and the P2A element can be termed an “fP2A element.” In some embodiments, a fP2A element refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 11; (ii) encodes the amino acid sequence of SEQ ID NO: 10; or (iii) encodes the amino acid sequence of SEQ ID NO: 10, comprising 1, 2, or 3 amino acid modifications. In some embodiments, the P2A element additionally comprises, at its 5′ end, a polynucleotide sequence that encodes a GSG (e.g., SEQ ID Nos: 20 and 21).

As used herein, the term “T2A element” refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 23, or 25; (ii) encodes the amino acid sequence of SEQ ID NO: 22, or 24; or (iii) encodes the amino acid sequence of SEQ ID NO: 22, or 24, comprising 1, 2, or 3 amino acid modifications. In some embodiments, when positioned in a vector between a first polynucleotide sequence encoding a first protein and a second polynucleotide sequence encoding a second protein, the T2A element is capable of mediating the translation of the first polynucleotide sequence and the second polynucleotide sequence as two distinct polypeptides from the same mRNA molecule by preventing the synthesis of a peptide bond, e.g., between the penultimate residue (e.g., glycine) and the ultimate residue (e.g., proline) at the C terminus of the translation product of the T2A element, e.g., such that the penultimate residue (e.g., glycine) becomes the C-terminal residue of the first protein and the ultimate residue (e.g., proline) becomes the N-terminal residue of the second protein. In some embodiments, the T2A element additionally comprises, at its 5′ end, a polynucleotide sequence that encodes a furin recognition site, e.g., RAKR (SEQ ID NO: 2). In some embodiments, the T2A element additionally comprises, at its 5′ end, a polynucleotide sequence that encodes a furin recognition site, e.g., RAKRSGSG (SEQ ID NO: 4), and the T2A element can be termed an “fT2A element.” In some embodiments, an fT2A element refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 13; (ii) encodes the amino acid sequence of SEQ ID NO: 12; or (iii) encodes the amino acid sequence of SEQ ID NO: 12, comprising 1, 2, or 3 amino acid modifications. In some embodiments, the T2A element additionally comprises, at its 5′ end, a polynucleotide sequence that encodes a GSG (e.g., SEQ ID Nos: 24 and 25).

As used herein, the term “F2A element” refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 27, or 29; (ii) encodes the amino acid sequence of SEQ ID NO: 26, or 28; or (iii) encodes the amino acid sequence of SEQ ID NO: 26, or 28, comprising 1, 2, or 3 amino acid modifications. In some embodiments, when positioned in a vector between a first polynucleotide sequence encoding a first protein and a second polynucleotide sequence encoding a second protein, the F2A element is capable of mediating the translation of the first polynucleotide sequence and the second polynucleotide sequence as two distinct polypeptides from the same mRNA molecule by preventing the synthesis of a peptide bond, e.g., between the penultimate residue (e.g., glycine) and the ultimate residue (e.g., proline) at the C terminus of the translation product of the F2A element, e.g., such that the penultimate residue (e.g., glycine) becomes the C-terminal residue of the first protein and the ultimate residue (e.g., proline) becomes the N-terminal residue of the second protein. In some embodiments, the F2A element additionally comprises, at its 5′ end, a polynucleotide sequence that encodes a furin recognition site, e.g., RAKR (SEQ ID NO: 2). In some embodiments, the F2A element additionally comprises, at its 5′ end, a polynucleotide sequence that encodes a furin recognition site, e.g., RAKRSGSG (SEQ ID NO: 4), and the F2A element can be termed an “fF2A element.” In some embodiments, a fF2A element refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 15; (ii) encodes the amino acid sequence of SEQ ID NO: 14; or (iii) encodes the amino acid sequence of SEQ ID NO: 14, comprising 1, 2, or 3 amino acid modifications. In some embodiments, the F2A element additionally comprises, at its 5′ end, a polynucleotide sequence that encodes a GSG (e.g., SEQ ID Nos: 28 and 29).

As used herein, the term “E2A element” refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 31, or 33; (ii) encodes the amino acid sequence of SEQ ID NO: 30, or 32; or (iii) encodes the amino acid sequence of SEQ ID NO: 30, or 32, comprising 1, 2, or 3 amino acid modifications. In some embodiments, when positioned in a vector between a first polynucleotide sequence encoding a first protein and a second polynucleotide sequence encoding a second protein, the E2A element is capable of mediating the translation of the first polynucleotide sequence and the second polynucleotide sequence as two distinct polypeptides from the same mRNA molecule by preventing the synthesis of a peptide bond, e.g., between the penultimate residue (e.g., glycine) and the ultimate residue (e.g., proline) at the C terminus of the translation product of the E2A element, e.g., such that the penultimate residue (e.g., glycine) becomes the C-terminal residue of the first protein and the ultimate residue (e.g., proline) becomes the N-terminal residue of the second protein. In some embodiments, the E2A element additionally comprises, at its 5′ end, a polynucleotide sequence that encodes a furin recognition site, e.g., RAKR (SEQ ID NO: 2). In some embodiments, the E2A element additionally comprises, at its 5′ end, a polynucleotide sequence that encodes a furin recognition site, e.g., RAKRSGSG (SEQ ID NO: 4), and the E2A element can be termed an “fE2A element.” In some embodiments, a fE2A element refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 17; (ii) encodes the amino acid sequence of SEQ ID NO: 16; or (iii) encodes the amino acid sequence of SEQ ID NO: 16, comprising 1, 2, or 3 amino acid modifications. In some embodiments, the E2A element additionally comprises, at its 5′ end, a polynucleotide sequence that encodes a GSG (e.g., SEQ ID Nos: 32 and 33).

Examples of 2A elements comprising furin recognition sites at their N-terminal/5′ ends are found below in Table 2. The 2A sites themselves are broken out in Table 3.

TABLE 2
Polynucleotide sequences of exemplary furin-2A elements and amino acid
sequences of translations thereof.

| | | SEQ |
|---|---|---|
| | | ID |
| Description | Sequence | NO: |
| Translation of Furin-P2A element | RAKRSGSGATNFSLLKQAGDVEENPGP | 10 |
| Furin-P2A element | CGGGCGAAACGCTCTGGAAGCGGAGCGACCAATTTCAGCCTGC | 11 |
| | TGAAGCAGGCGGGCGATGTGGAGGAGAACCCTGGCCCA | |
| Translation of Furin-T2A element | RAKRSGSGEGRGSLLTCGDVEENPGP | 12 |
| Furin-T2A element | CGGGCGAAACGCTCTGGAAGCGGAGAGGGCAGAGGAAGTCTTC | 13 |
| | TAACATGCGGTGACGTGGAGGAGAATCCCGGCCCT | |
| Translation of Furin-F2A element | RAKRSGSGVKQTLNFDLLKLAGDVESNPGP | 14 |
| Furin-F2A element | CGGGCGAAACGCTCTGGAAGCGGAGTGAAGCAGACCCTGAATT | 15 |
| | TCGACCTGCTGAAGCTGGCCGGGGACGTGGAGAGCAACCCTGG | |
| | CCCC | |
| Translation of Furin-E2A element | RAKRSGSGQCTNYALLKLAGDVESNPGP | 16 |
| Furin-E2A element | CGGGCGAAACGCTCTGGAAGCGGACAGTGTACTAATTATGCTC | 17 |
| | TCTTGAAATTGGCTGGAGATGTTGAGAGCAACCCAGGTCCC | |

TABLE 3
Amino acid and polynucleotide sequences of exemplary 2A elements.

| | | SEQ |
|---|---|---|
| | | ID |
| Description | Amino Acid Sequence | NO: |
| P2A (exemplary amino acid | ATNFSLLKQAGDVEENPGP | 18 |
| sequence) | | |
| P2A (exemplary nucleotide | GCGACCAATTTCAGCCTGCTGAAGCAGGCGGGCGATGTGGAGG | 19 |
| sequence) | AGAACCCTGGCCCA | |
| P2A (with flanking residues) | GSGATNFSLLKQAGDVEENPGP | 20 |
| (exemplary amino acid sequence) | | |
| P2A (with flanking residues) | GGCTCCGGAGCGACCAATTTCAGCCTGCTGAAGCAGGCGGGCG | 21 |
| (exemplary nucleotide sequence) | ATGTGGAGGAGAACCCTGGCCCA | |
| T2A (exemplary amino acid | EGRGSLLTCGDVEENPGP | 22 |
| sequence) | | |
| T2A (exemplary nucleotide | GAGGGCAGAGGAAGTCTTCTAACATGCGGTGACGTGGAGGAGA | 23 |
| sequence) | ATCCCGGCCCT | |
| T2A (with flanking residues) | GSGEGRGSLLTCGDVEENPGP | 24 |
| (exemplary amino acid sequence) | | |
| T2A (with flanking residues) | GGCTCCGGAGAGGGCAGAGGAAGTCTTCTAACATGCGGTGACG | 25 |
| (exemplary nucleotide sequence) | TGGAGGAGAATCCCGGCCCT | |
| F2A (exemplary amino acid | VKQTLNFDLLKLAGDVESNPGP | 26 |
| sequence) | | |
| F2A (exemplary nucleotide | GTGAAGCAGACCCTGAATTTCGACCTGCTGAAGCTGGCCGGGG | 27 |
| sequence) | ACGTGGAGAGCAACCCTGGCCCC | |
| F2A (with flanking residues) | GSGVKQTLNFDLLKLAGDVESNPGP | 28 |
| (exemplary amino acid sequence) | | |
| F2A (with flanking residues) | GGCTCCGGAGTGAAGCAGACCCTGAATTTCGACCTGCTGAAGC | 29 |
| (exemplary nucleotide sequence) | TGGCCGGGGACGTGGAGAGCAACCCTGGCCCC | |
| E2A (exemplary amino acid | QCTNYALLKLAGDVESNPGP | 30 |
| sequence) | | |
| E2A (exemplary nucleotide | CAGTGTACTAATTATGCTCTCTTGAAATTGGCTGGAGATGTTG | 31 |
| sequence) | AGAGCAACCCAGGTCCC | |
| E2A (with flanking residues) | GSGQCTNYALLKLAGDVESNPGP | 32 |
| (exemplary amino acid sequence) | | |
| E2A (with flanking residues) | GGCTCCGGACAGTGTACTAATTATGCTCTCTTGAAATTGGCTG | 33 |
| (exemplary nucleotide sequence) | GAGATGTTGAGAGCAACCCAGGTCCC | |

As used herein, the terms “inverted terminal repeat,” “ITR,” “inverted repeat/direct repeat,” and “IR/DR” are used interchangeably and refer to a polynucleotide sequence, e.g., of about 230 nucleotides (e.g., 220, 221, 222, 223, 224, 225, 226, 227, 228, 229, 230, 231, 232, 233, 234, 235, 236, 237, 238, 239, or 240 nucleotides), flanking (e.g., with or without an intervening polynucleotide sequence) one end of an expression cassette (e.g., a polycistronic expression cassette) that can be cleaved by a transposase polypeptide when used in combination with a corresponding, e.g., reverse-complementary (e.g., perfectly or imperfectly reverse-complementary) polynucleotide sequence, e.g., of about 230 nucleotides (e.g., 220, 221, 222, 223, 224, 225, 226, 227, 228, 229, 230, 231, 232, 233, 234, 235, 236, 237, 238, 239, or 240 nucleotides), flanking (e.g., with or without an intervening polynucleotide sequence) the opposite end of the expression cassette (e.g., a polycistronic expression cassette) (e.g., as described in Cui et al., J. Mol. Biol. 2002; 318(5):1221-35, the contents of which are incorporated by reference in their entirety herein). In some embodiments, an ITR, e.g., an ITR of a DNA transposon (e.g., a Sleeping Beauty transposon, a piggyBac transposon, a TcBuster transposon, and a Tol2 transposon) contains two direct repeats (“DRs”), e.g., imperfect direct repeats, e.g., of about 30 nucleotides (e.g., 25, 26, 27, 28, 29, 30, 31, 32, 33, 34, or 35 nucleotides), located at each end of the ITR. The terms “ITR” and “DR,” when used in reference to a single- or double-stranded DNA vector, refer to the DNA sequence of the sense strand. A transposase polypeptide may recognize the sense strand and/or the antisense strand of DNA.

As used herein, the term “Left ITR,” when used in reference to a linear single- or double-stranded DNA vector, refers to the ITR positioned 5′ of the polycistronic expression cassette. As used herein, the term “Right ITR,” when used in reference to a linear single- or double-stranded DNA vector, refers to the ITR positioned 3′ of the polycistronic expression cassette. When a circular vector is used, the Left ITR is closer than the Right ITR to the 5′ end of the polycistronic expression cassette, and the Right ITR is closer than the Left ITR to the 3′ end of the polycistronic expression cassette.

As used herein, the term “operably linked” refers to a linkage of polynucleotide sequence elements or amino acid sequence elements in a functional relationship. For example, a polynucleotide sequence is operably linked when it is placed into a functional relationship with another polynucleotide sequence. In some embodiments, a transcription regulatory polynucleotide sequence e.g., a promoter, enhancer, or other expression control element is operably linked to a polynucleotide sequence that encodes a protein if it affects the transcription of the polynucleotide sequence that encodes the protein.

The term “polynucleotide” as used herein refers to a polymer of DNA or RNA. The polynucleotide sequence can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified polynucleotide sequence. Polynucleotide sequences include, but are not limited to, all polynucleotide sequences which are obtained by any means available in the art, including, without limitation, recombinant means, e.g., the cloning of polynucleotide sequences from a recombinant library or a cell genome, using ordinary cloning technology and polymerase chain reaction, and the like, and by synthetic means.

The terms “protein” and “polypeptide” are used interchangeably herein and refer to a polymer of amino acids connected by one or more peptide bonds. As used herein, “amino acid sequence” refers to the information describing the relative order and identity of amino acid residues which make up a polypeptide.

The term “functional variant” as used herein in reference to a protein or polypeptide refers to a protein that comprises at least one amino acid modification (e.g., a substitution, deletion, addition) compared to the amino acid sequence of a reference protein, that retains at least one particular function. In some embodiments, the reference protein is a wild type protein. For example, a functional variant of an IL-15 protein can refer to an IL-15 protein comprising an amino acid substitution compared to a wild type IL-15 protein that retains the ability to bind the IL-15 receptor a chain (IL-15Rα). Not all functions of the reference wild type protein need be retained by the functional variant of the protein. In some instances, one or more functions are selectively reduced or eliminated.

The term “functional fragment” as used herein in reference to a protein or polypeptide refers to a fragment of a reference protein that retains at least one particular function. For example, a functional fragment of an IL-15 protein can refer to a fragment of the protein that retains the ability to specifically bind IL-15Rα. Not all functions of the reference protein need be retained by a functional fragment of the protein. In some instances, one or more functions are selectively reduced or eliminated.

As used herein, the term “modification,” with reference to a polynucleotide sequence, refers to a polynucleotide sequence that comprises at least one substitution, alteration, inversion, addition, or deletion of nucleotide compared to a reference polynucleotide sequence. As used herein, the term “modification,” with reference to an amino acid sequence, refers to an amino acid sequence that comprises at least one substitution, alteration, inversion, addition, or deletion of an amino acid residue compared to a reference amino acid sequence.

As used herein, the term “derived from,” with reference to a polynucleotide sequence, refers to a polynucleotide sequence that has at least 85% sequence identity to a reference naturally occurring nucleic acid sequence from which it is derived. The term “derived from,” with reference to an amino acid sequence, refers to an amino acid sequence that has at least 85% sequence identity to a reference naturally occurring amino acid sequence from which it is derived. The term “derived from” as used herein does not denote any specific process or method for obtaining the polynucleotide or amino acid sequence. For example, the polynucleotide or amino acid sequence can be chemically synthesized.

As used herein, the term “linked to” refers to covalent or noncovalent binding between two molecules or moieties. The skilled worker will appreciate that when a first molecule or moiety is linked to a second molecule or moiety, the linkage need not be direct, but instead, can be via an intervening molecule or moiety.

As used herein, the term “marker protein” or “marker polypeptide” are used interchangeably and refer to a protein or polypeptide that can be expressed on the surface of a cell, which can be utilized to mark or deplete cells expressing the marker protein or polypeptide. In some embodiments, depletion of cells expressing the marker protein or polypeptide is performed through the administration of a molecule that specifically binds the marker protein or polypeptide (e.g., an antibody that mediates antibody dependent cellular cytotoxicity).

As used herein, the term “immune effector cell” refers to a cell that is involved in the promotion of an immune effector function. Examples of immune effector cells include, but are not limited to, T cells (e.g., alpha/beta T cells and gamma/delta T cells, CD4⁺ T cells, CD8⁺ T cells, natural killer T (NKT) cells), natural killer (NK) cells, B cells, mast cells, and myeloid-derived phagocytes.

As used herein, the term “immune effector function” refers to a specialized function of an immune effector cell. The effector function ofany given immune effector cell can be different. For example, an effector function of a CDA+ T cell is cytolytic activity, and an effector function of a CD4+ T cell is secretion of a cytokine.

### 1.2 T Cell Receptors

In one aspect, the instant disclosure provides electroporated T cells that express TCRs via a polycistronic expression cassette. In certain embodiments, the TCR comprises a T cell receptor (TCR) alpha chain comprising an alpha chain variable (Vα) region and an alpha chain constant (Cα) region and a TCR beta chain comprising a beta chain variable (Vβ) region and a beta chain constant (Cβ). The amino acid sequences of constant domains comprised in the TCRs disclosed herein are shown in Tables 4 and 5 below.

TABLE 4
Amino acid sequences of TCR Cα regions.

| | | SEQ |
|---|---|---|
| | | ID |
| Description | Sequence | NO: |
| Cα (murine, | XIQNPEPAVYQLKDPRSQDSTLCLFTDFDSQINVPKTMESGTFITDKXVLDM | 40 |
| degenerate) | KAMDSKSNGAIAWSNQTSFTCQDIFKETNATYPSSDVPCDATLTEKSFETDM | |
| | NLNFQNLXVXXLRILLLKVAGENLLMTLRLWSS | |
| | X at position 1 is Asn, Asp, His, or Tyr; | |
| | X at position 48 is Thr or Cys; | |
| | X at position 112 is Ser, Ala, Vαl, Leu, Ile, Pro, Phe, Met, or Trp; | |
| | X at position 114 is Met, Ala, Vαl, Leu, Ile, Pro, Phe, or Trp; | |
| | X at position 115 is Gly, Ala, Vαl, Leu, Ile, Pro, Phe, Met, or Trp | |
| Cα (murine, | NNNATCCAGAATCCCGAGCCTGCGGTGTACCAGCTGAAGGACCCCCGCTCTC | 57 |
| degenerate) | AGGATAGCACACTGTGCCTGTTCACCGACTTTGATAGCCAGATCAACGTGCC | |
| (exemplary nucleotide | TAAAACAATGGAGTCCGGCACCTTCATCACCGACAAGNNNGTGCTGGATATG | |
| sequence) | AAAGCGATGGACTCCAAGTCTAACGGCGCGATCGCGTGGTCCAATCAGACAT | |
| | CTTTCACCTGCCAGGATATCTTCAAGGAGACAAACGCGACCTATCCTTCCTC | |
| | TGACGTGCCATGTGATGCGACACTGACCGAGAAGAGCTTCGAGACAGACATG | |
| | AACCTGAATTTTCAGAATCTGNNNGTCNNNNNNCTGAGAATCCTGCTGCTGA | |
| | AGGTGGCGGGCTTTAATCTGCTGATGACACTGCGGCTGTGGAGTTCC | |
| | NNN at positions 1-3 make up a codon that encodes Asn, Asp, His, or Tyr; | |
| | NNN at positions 142-144 make up a codon that encodes Thr or Cys; | |
| | NNN at positions 334-336 make up a codon that encodes Ser, Ala, Vαl, Leu, | |
| | Ile, Pro, Phe, Met, or Trp; | |
| | NNN at positions 340-342 make up a codon that encodes Met, Ala, Vαl, Leu, | |
| | Ile, Pro, Phe, or Trp; | |
| | NNN at positions 343-345 make up a codon that encodes Gly, Ala, Vαl, Leu, | |
| | Ile, Pro, Phe, Met, or Trp | |
| Cα (murine, cysteine- | NIQNPEPAVYQLKDPRSQDSTLCLFTDFDSQINVPKTMESGTFITDKCVLDM | 41 |
| and LIV-substituted) | KAMDSKSNGAIAWSNQTSFTCQDIFKETNATYPSSDVPCDATLTEKSFETDM | |
| | NLNFQNLLVIVLRILLLKVAGFNLLMTLRLWSS | |
| Cα (murine, cysteine- | AACATCCAGAATCCCGAGCCTGCGGTGTACCAGCTGAAGGACCCCCGCTCTC | 55 |
| and LIV-substituted) | AGGATAGCACACTGTGCCTGTTCACCGACTTTGATAGCCAGATCAACGTGCC | |
| (exemplary nucleotide | TAAAACAATGGAGTCCGGCACCTTCATCACCGACAAGTGCGTGCTGGATATG | |
| sequence) | AAAGCGATGGACTCCAAGTCTAACGGCGCGATCGCGTGGTCCAATCAGACAT | |
| | CTTTCACCTGCCAGGATATCTTCAAGGAGACAAACGCGACCTATCCTTCCTC | |
| | TGACGTGCCATGTGATGCGACACTGACCGAGAAGAGCTTCGAGACAGACATG | |
| | AACCTGAATTTTCAGAATCTGCTGGTCATCGTGCTGAGAATCCTGCTGCTGA | |
| | AGGTGGCGGGCTTTAATCTGCTGATGACACTGCGGCTGTGGAGTTCC | |
| Cα (murine, LIV | NIQNPEPAVYQLKDPRSQDSTLCLFTDFDSQINVPKTMESGTFITDKTVLDM | 42 |
| substituted) | KAMDSKSNGAIAWSNQTSFTCQDIFKETNATYPSSDVPCDATLTEKSFETDM | |
| | NLNFQNLLVIVLRILLLKVAGFNLLMTLRLWSS | |
| Cα (murine, LIV | AACATCCAGAATCCCGAGCCTGCGGTGTACCAGCTGAAGGACCCCCGCTCTC | 58 |
| substituted) | AGGATAGCACACTGTGCCTGTTCACCGACTTTGATAGCCAGATCAACGTGCC | |
| (exemplary nucleotide | TAAAACAATGGAGTCCGGCACCTTCATCACCGACAAGACCGTGCTGGATATG | |
| sequence) | AAAGCGATGGACTCCAAGTCTAACGGCGCGATCGCGTGGTCCAATCAGACAT | |
| | CTTTCACCTGCCAGGATATCTTCAAGGAGACAAACGCGACCTATCCTTCCTC | |
| | TGACGTGCCATGTGATGCGACACTGACCGAGAAGAGCTTCGAGACAGACATG | |
| | AACCTGAATTTTCAGAATCTGCTGGTCATCGTGCTGAGAATCCTGCTGCTGA | |
| | AGGTGGCGGGCTTTAATCTGCTGATGACACTGCGGCTGTGGAGTTCC | |
| Cα (murine, cysteine- | NIQNPEPAVYQLKDPRSQDSTLCLFTDFDSQINVPKTMESGTFITDKCVLDM | 43 |
| substituted) | KAMDSKSNGAIAWSNQTSFTCQDIFKETNATYPSSDVPCDATLTEKSFETDM | |
| | NLNFQNLSVMGLRILLLKVAGFNLLMTLRLWSS | |
| Cα (murine, wild type) | NIQNPEPAVYQLKDPRSQDSTLCLFTDFDSQINVPKTMESGTFITDKTVLDM | 44 |
| | KAMDSKSNGAIAWSNQTSFTCQDIFKETNATYPSSDVPCDATLTEKSFETDM | |
| | NLNFQNLSVMGLRILLLKVAGFNLLMTLRLWSS | |
| Cα (human, | XIQNPDPAVYQLRDSKSSDKSVCLFTDFDSQTNVSQSKDSDVYITDKXVLDM | 45 |
| degenerate) | RSMDFKSNSAVAWSNKSDFACANAFNNSIIPEDTFFPSPESSCDVKLVEKSF | |
| | ETDTNLNFQNLXVXXFRILLLKVAGFNLLMTLRLWSS | |
| | X at position 1 is Asn, Asp, His, or Tyr | |
| | X at position 48 is Thr or Cys; | |
| | X at position 116 is Ser, Ala, Vαl, Leu, Ile, Pro, Phe, Met, or Trp; | |
| | X at position 118 is Met, Ala, Vαl, Leu, Ile, Pro, Phe, or Trp; | |
| | X at position 119 is Gly, Ala, Vαl, Leu, Ile, Pro, Phe, Met, or Trp | |
| Cα (human, cysteine- | XIQNPDPAVYQLRDSKSSDKSVCLFTDFDSQTNVSQSKDSDVYITDKCVLDM | 46 |
| and LIV-substituted; | RSMDFKSNSAVAWSNKSDFACANAFNNSIIPEDTFFPSPESSCDVKLVEKSF | |
| degenerate at position | ETDTNLNFQNLLVIVFRILLLKVAGFNLLMTLRLWSS | |
| 1) | X at position 1 is Asn, Asp, His, or Tyr | |
| Cα (human, LIV- | XIQNPDPAVYQLRDSKSSDKSVCLFTDFDSQTNVSQSKDSDVYITDKTVLDM | 47 |
| substituted; degenerate | RSMDFKSNSAVAWSNKSDFACANAFNNSIIPEDTFFPSPESSCDVKLVEKSF | |
| at position 1) | ETDTNLNFQNLLVIVFRILLLKVAGFNLLMTLRLWSS | |
| | X at position 1 is Asn, Asp, His, or Tyr | |
| Cα (human, cysteine- | XIQNPDPAVYQLRDSKSSDKSVCLFTDFDSQTNVSQSKDSDVYITDKCVLDM | 48 |
| substituted; degenerate | RSMDFKSNSAVAWSNKSDFACANAFNNSIIPEDTFFPSPESSCDVKLVEKSF | |
| at position 1) | ETDTNLNFQNLSVIGFRILLLKVAGFNLLMTLRLWSS | |
| | X at position 1 is Asn, Asp, His, or Tyr | |
| Cα (human, wild type; | XIQNPDPAVYQLRDSKSSDKSVCLFTDFDSQTNVSQSKDSDVYITDKTVLDM | 49 |
| degenerate at position | RSMDFKSNSAVAWSNKSDFACANAFNNSIIPEDTFFPSPESSCDVKLVEKSF | |
| 1) | ETDTNLNFQNLSVIGFRILLLKVAGFNLLMTLRLWSS | |
| | X at position 1 is Asn, Asp, His, or Tyr | |

TABLE 5
Amino acid sequences of TCR Cβ regions.

| | | SEQ |
|---|---|---|
| | | ID |
| Description | Sequence | NO: |
| Cβ (murine, | EDLRNVTPPKVSLFEPSKAEIANKQKATLVCLARGFFPDHVELSWWVNGKEV | 50 |
| degenerate) | HSGVXTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNS | |
| | X at position 57 is Ser or Cys | |
| Cβ (murine, | GAGGACCTGAGGAACGTGACCCCACCTAAAGTGAGCCTGTTCGAGCCATCCA | 59 |
| degenerate) | AGGCGGAGATCGCGAATAAGCAGAAAGCGACCCTGGTGTGCCTGGCGAGGGG | |
| (exemplary nucleotide | CTTCTTTCCCGATCACGTGGAGCTGTCCTGGTGGGTGAACGGCAAAGAGGTG | |
| sequence) | CACTCTGGCGTGNNNACAGACCCTCAGGCGTACAAGGAGAGCAATTACTCCT | |
| | ATTGTCTGTCTAGCAGACTGAGGGTGAGCGCGACCTTTTGGCACAACCCCCG | |
| | GAATCACTTCCGCTGCCAGGTGCAGTTTCACGGCCTGTCCGAGGAGGATAAA | |
| | TGGCCTGAGGGCTCTCCAAAGCCCGTGACACAGAATATCAGCGCGGAGGCGT | |
| | GGGGAAGAGCGGACTGTGGCATTACAAGCGCGTCCTATCAGCAGGGCGTGCT | |
| | GTCCGCGACCATCCTGTACGAGATTCTGCTGGGCAAGGCGACACTGTATGCG | |
| | GTGCTGGTGTCCACCCTGGTGGTCATGGCGATGGTGAAGAGGAAAAACTCT | |
| | NNN at positions 169-171 make up a codon that encodes Ser or Cys | |
| Cβ (murine, cysteine- | EDLRNVTPPKVSLFEPSKAEIANKQKATLVCLARGFFPDHVELSWWVNGKEV | 51 |
| substituted) | HSGVCTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNS | |
| Cβ (murine, cysteine- | GAGGACCTGAGGAACGTGACCCCACCTAAAGTGAGCCTGTTCGAGCCATCCA | 56 |
| substituted) | AGGCGGAGATCGCGAATAAGCAGAAAGCGACCCTGGTGTGCCTGGCGAGGGG | |
| (exemplary nucleotide | CTTCTTTCCCGATCACGTGGAGCTGTCCTGGTGGGTGAACGGCAAAGAGGTG | |
| sequence) | CACTCTGGCGTGTGCACAGACCCTCAGGCGTACAAGGAGAGCAATTACTCCT | |
| | ATTGTCTGTCTAGCAGACTGAGGGTGAGCGCGACCTTTTGGCACAACCCCCG | |
| | GAATCACTTCCGCTGCCAGGTGCAGTTTCACGGCCTGTCCGAGGAGGATAAA | |
| | TGGCCTGAGGGCTCTCCAAAGCCCGTGACACAGAATATCAGCGCGGAGGCGT | |
| | GGGGAAGAGCGGACTGTGGCATTACAAGCGCGTCCTATCAGCAGGGCGTGCT | |
| | GTCCGCGACCATCCTGTACGAGATTCTGCTGGGCAAGGCGACACTGTATGCG | |
| | GTGCTGGTGTCCACCCTGGTGGTCATGGCGATGGTGAAGAGGAAAAACTCT | |
| Cβ (murine, wild type) | EDLRNVTPPKVSLFEPSKAEIANKQKATLVCLARGFFPDHVELSWWVNGKEV | 52 |
| | HSGVSTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNS | |
| Cβ (human, | EDLKNVFPPEVAVFEPSEAEISHTQKATLVCLATGFYPDHVELSWWVNGKEV | 53 |
| degenerate) | HSGVXTDPQPLKEQPALNDSRYCLSSRLRVSATFWQNPRNHFRCQVQFYGLS | |
| | ENDEWTQDRAKPVTQIVSAEAWGRADCGFTSESYQQGVLSATILYEILLGKA | |
| | TLYAVLVSALVLMAMVKRKDSRG | |
| | X at position 57 is Ser or Cys | |
| Cβ (human, cysteine- | EDLKNVFPPEVAVFEPSEAEISHTQKATLVCLATGFYPDHVELSWWVNGKEV | 54 |
| substituted) | HSGVCTDPQPLKEQPALNDSRYCLSSRLRVSATFWQNPRNHFRCQVQFYGLS | |
| | ENDEWTQDRAKPVTQIVSAEAWGRADCGFTSESYQQGVLSATILYEILLGKA | |
| | TLYAVLVSALVLMAMVKRKDSRG | |
| Cβ (human, wild type) | EDLKNVFPPEVAVFEPSEAEISHTQKATLVCLATGFYPDHVELSWWVNGKEV | 60 |
| | HSGVSTDPQPLKEQPALNDSRYCLSSRLRVSATFWQNPRNHFRCQVQFYGLS | |
| | ENDEWTQDRAKPVTQIVSAEAWGRADCGFTSESYQQGVLSATILYEILLGKA | |
| | TLYAVLVSALVLMAMVKRKDSRG | |

As used herein, “LIV-substituted” refers to a Cα sequence disclosed herein which, relative to SEQ ID NO: 40, comprises a leucine residue at position 112, an isoleucine residue at position 114, and a valine residue at position 115. See, for example, SEQ ID Nos: 41 and 42. In some embodiments, and independent of the LIV-substitutions a Cα sequence disclosed herein can comprise a cysteine at position 48, replacing the threonine residue. (Compare SEQ ID Nos: 40-44). In some embodiments, the Cβ sequence disclosed herein has a substitution of the serine at residue 57 with cysteine. This is shown in SEQ ID Nos: 50 and 51.

Tumor Protein p53 (also referred to as “p53”) acts as a tumor suppressor by, for example, regulating cell division. In some embodiments, wild type full-length p53 has the amino acid sequence of SEQ ID NO: 340, shown below.

(SEQ ID NO: 340)
MEEPQSDPSVEPPLSQETFSDLWKLLPENNVLSPLPSQAMDDLMLSPDDI
EQWFTEDPGPDEAPRMPEAAPPVAPAPAAPTPAAPAPAPSWPLSSSVPSQ
KTYQGSYGFRLGFLHSGTAKSVTCTYSPALNKMFCQLAKTCPVQLWVDST
PPPGTRVRAMAIYKQSQHMTEVVRRCPHHERCSDSDGLAPPQHLIRVEGN
LRVEYLDDRNTFRHSVVVPYEPPEVGSDCTTIHYNYMCNSSCMGGMNRRP
ILTIITLEDSSGNLLGRNSFEVRVCACPGRDRRTEEENLRKKGEPHHELP
PGSTKRALPNNTSSSPQPKKKPLDGEYFTLQIRGRERFEMFRELNEALEL
KDAQAGKEPGGSRAHSSHLKSKKGQSTSRHKKLMFKTEGPDSD

Kirsten rat sarcoma viral oncogene homolog (KRAS), also referred to as GTPase Kras, V-Ki-Ras2 Kirsten rat sarcoma viral oncogene, or KRAS2, is a member of the small GTPase superfamily. There are two transcript variants of KRAS: KRAS variant A and KRAS variant B. Hereinafter, references to “KRAS” (mutated or unmutated) refer to both variant A and variant B, unless specified otherwise. In some embodiments, wild type KRAS variant A has the amino acid sequence of SEQ ID NO: 341 and wild type KRAS variant B has the amino acid sequence of SEQ ID NO: 342, both shown below.

(SEQ ID NO: 341)
MTEYKLVVVGAGGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
RVEDAFYTLVREIRQYRLKKISKEEKTPGCVKIKKCIIM
(SEQ ID NO: 342)
MTEYKLVVVGAGGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM

EGFR (also referred to as ERBB1 or HER1) is a transmembrane glycoprotein that belongs to the receptor tyrosine kinase (RTK) super-family of cell surface receptors, which mediate cell signaling by extra-cellular growth factors. Examples of wild type (WT), unmutated human EGFR amino acid sequences include those disclosed in GenBank Accession Nos. NP_001 333826.1 (isoform e precursor), NP_001333827.1 (isoform f precursor), NP_001333828.1 (isoform g precursor), NP_001333829.1 (isoform h precursor), NP_001333870.1 (isoform i precursor), NP_005219.2 (isoform a precursor), NP_958439.1 (isoform b precursor), NP_958440.1 (isoform c precursor), and NP_958441.1 (isoform d precursor). In some embodiments, wild type EGFR has the amino acid sequence of SEQ ID NO: 343

(SEQ ID NO: 343)
MRPSGTAGAALLALLAALCPASRALEEKKVCQGTSNKLTQLGTFEDHFLS
LQRMFNNCEVVLGNLEITYVQRNYDLSFLKTIQEVAGYVLIALNTVERIP
LENLQIIRGNMYYENSYALAVLSNYDANKTGLKELPMRNLQEILHGAVRF
SNNPALCNVESIQWRDIVSSDFLSNMSMDFQNHLGSCQKCDPSCPNGSCW
GAGEENCQKLTKIICAQQCSGRCRGKSPSDCCHNQCAAGCTGPRESDCLV
CRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLS
INATNIKHFKNCTSISGDLHILPVAFRGDSFTHTPPLDPQELDILKTVKE
ITGFLLIQAWPENRTDLHAFENLEIIRGRTKQHGQFSLAVVSLNITSLGL
RSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFV
ENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGM
VGALLLLLVVALGIGLFMRRRHIVRKRTLRRLLQERELVEPLTPSGEAPN
QALLRILKETEFKKIKVLGSGAFGTVYKGLWIPEGEKVKIPVAIKELREA
TSPKANKEILDEAYVMASVDNPHVCRLLGICLTSTVQLITQLMPFGCLLD
YVREHKDNIGSQYLLNWCVQIAKGMNYLEDRRLVHRDLAARNVLVKTPQH
VKITDFGLAKLLGAEEKEYHAEGGKVPIKWMALESILHRIYTHQSDVWSY
GVTVWELMTFGSKPYDGIPASEISSILEKGERLPQPPICTIDVYMIMVKC
WMIDADSRPKFRELIIEFSKMARDPQRYLVIQGDERMHLPSPTDSNFYRA
LMDEEDMDDVVDADEYLIPQQGFFSSPSTSRTPLLSSLSATSNNSTVACI
DRNGLQSCPIKEDSFLQRYSSDPTGALTEDSIDDTFLPVPEYINQSVPKR
PAGSVQNPVYHNQPLNPAPSRDPHYQDPHSTAVGNPEYLNTVQPTCVNST
FDSPAHWAQKGSHQISLDNPDYQQDFFPKEAKPNGIFKGSTAENAEYLRV
APQSSEFIGA

The amino acid sequences of exemplary TCRs are set forth in Table 6 herein.

TABLE 6A
Amino acid sequences of TCR001.

| | | SEQ |
|---|---|---|
| | | ID |
| Description | Sequence | NO: |
| CDR1α | NYSPAY | 1001 |
| CDR2α | IRENEKE | 1002 |
| CDR3α | ALDIYPHDMR | 1003 |
| Vα without signal | QKIEQNSEALNIQEGKTATLTCNYTNYSPAYLQWYRQDPGRGPVFLLLIREN | 1004 |
| peptide (SignalP) | EKEKRKERLKVTFDTTLKQSLFHITASQPADSATYLCALDIYPHDMRFGAGT | |
| | RLTVKP | |
| Vα without signal | SQKIEQNSEALNIQEGKTATLTCNYTNYSPAYLQWYRQDPGRGPVFLLLIRE | 1005 |
| peptide (IMGT) | NEKEKRKERLKVTFDTTLKQSLFHITASQPADSATYLCALDIYPHDMRFGAG | |
| | TRLTVKP | |
| Vα | MXSFLGGVLLILWLQVDWVKSQKIEQNSEALNIQEGKTATLTCNYTNYSPAY | 1006 |
| | LQWYRQDPGRGPVFLLLIRENEKEKRKERLKVTFDTTLKQSLFHITASQPAD | 1007 |
| | SATYLCALDIYPHDMRFGAGTRLTVKP | |
| | (X = any amino acid) | |
| α chain with WT signal | MESFLGGVLLILWLQVDWVKSQKIEQNSEALNIQEGKTATLTCNYTNYSPAY | 1008 |
| peptide, Cα | LQWYRQDPGRGPVFLLLIRENEKEKRKERLKVTFDTTLKQSLFHITASQPAD | |
| (substituted) | SATYLCALDIYPHDMRFGAGTRLTVKPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain with | MASFLGGVLLILWLQVDWVKSQKIEQNSEALNIQEGKTATLTCNYTNYSPAY | 1009 |
| alternative signal | LQWYRQDPGRGPVFLLLIRENEKEKRKERLKVTFDTTLKQSLFHITASQPAD | |
| peptide, Cα | SATYLCALDIYPHDMRFGAGTRLTVKPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain with | MHSFLGGVLLILWLQVDWVKSQKIEQNSEALNIQEGKTATLTCNYTNYSPAY | 1010 |
| alternative signal | LQWYRQDPGRGPVFLLLIRENEKEKRKERLKVTFDTTLKQSLFHITASQPAD | |
| peptide, Cα | SATYLCALDIYPHDMRFGAGTRLTVKPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| CDR1β | SGHAT | 2001 |
| CDR2β | FQNNGV | 2002 |
| CDR3β | ASSLDPGDTGELF | 2003 |
| Vβ without signal | GVAQSPRYKIIEKRQSVAFWCNPISGHATLYWYQQILGQGPKLLIQFQNNGV | 2004 |
| peptide (SignalP) | VDDSQLPKDRFSAERLKGVDSTLKIQPAKLEDSAVYLCASSLDPGDTGELFF | |
| | GEGSRLTVL | |
| Vβ without signal | EAGVAQSPRYKIIEKRQSVAFWCNPISGHATLYWYQQILGQGPKLLIQFQNN | 2005 |
| peptide (IMGT) | GVVDDSQLPKDRFSAERLKGVDSTLKIQPAKLEDSAVYLCASSLDPGDTGEL | |
| | FFGEGSRLTVL | |
| Vβ | MXTRLLCWAALCLLGAELTEAGVAQSPRYKIIEKRQSVAFWCNPISGHATLY | 2006 |
| | WYQQILGQGPKLLIQFQNNGVVDDSQLPKDRFSAERLKGVDSTLKIQPAKLE | 2007 |
| | DSAVYLCASSLDPGDTGELFFGEGSRLTVL | |
| | (X = any amino acid) | |
| β chain with WT signal | MGTRLLCWAALCLLGAELTEAGVAQSPRYKIIEKRQSVAFWCNPISGHATLY | 2008 |
| peptide, Cβ | WYQQILGQGPKLLIQFQNNGVVDDSQLPKDRFSAERLKGVDSTLKIQPAKLE | |
| (substituted) | DSAVYLCASSLDPGDTGELFFGEGSRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLCWAALCLLGAELTEAGVAQSPRYKIIEKRQSVAFWCNPISGHATLY | 2009 |
| signal peptide, Cβ | WYQQILGQGPKLLIQFQNNGVVDDSQLPKDRFSAERLKGVDSTLKIQPAKLE | |
| (substituted) | DSAVYLCASSLDPGDTGELFFGEGSRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLCWAALCLLGAELTEAGVAQSPRYKIIEKRQSVAFWCNPISGHATLY | 2010 |
| signal peptide, Cβ | WYQQILGQGPKLLIQFQNNGVVDDSQLPKDRFSAERLKGVDSTLKIQPAKLE | |
| (substituted) | DSAVYLCASSLDPGDTGELFFGEGSRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR001 interacts with and/or is specific for a peptide from the tumor protein p53 (p53). In some embodiments, the peptide is from a neoantigen of p53 and has the amino acid change R175H (in which position 175 of the p53 protein is mutated from Arg to His). In some embodiments, TCR001 interacts with and/or is specific for the neoantigen in the context of HLA-A*02:01, as described in International Publication No. WO 2019/067243, incorporated herein by reference in its entirety.

TABLE 6B
Amino acid sequences of TCR002.

| | | SEQ |
|---|---|---|
| | | ID |
| Description | Sequence | NO: |
| CDR1α | DSASNY | 1011 |
| CDR2α | IRSNVGE | 1012 |
| CDR3α | AASKSAIMVVLQTSSSL | 1013 |
| Vα without signal | ENVEQHPSTLSVQEGDSAVIKCTYSDSASNYFPWYKQELGKGPQLIIDIRSN | 1014 |
| peptide (SignalP) | VGEKKDQRIAVTLNKTAKHFSLHITETQPEDSAVYFCAASKSAIMVVLQTSS | |
| | SLELALCLLSSQV | |
| Vα without signal | GENVEQHPSTLSVQEGDSAVIKCTYSDSASNYFPWYKQELGKGPQLIIDIRS | 1015 |
| peptide (IMGT) | NVGEKKDQRIAVTLNKTAKHFSLHITETQPEDSAVYFCAASKSAIMVVLQTS | |
| | SSLELALCLLSSQV | |
| Vα | MXSIRAVFIFLWLQLDLVNGENVEQHPSTLSVQEGDSAVIKCTYSDSASNYF | 1016 |
| | PWYKQELGKGPQLIIDIRSNVGEKKDQRIAVTLNKTAKHFSLHITETQPEDS | 1017 |
| | AVYFCAASKSAIMVVLQTSSSLELALCLLSSQV | |
| | (X = any amino acid) | |
| α chain with WT signal | MTSIRAVFIFLWLQLDLVNGENVEQHPSTLSVQEGDSAVIKCTYSDSASNYF | 1018 |
| peptide, Cα | PWYKQELGKGPQLIIDIRSNVGEKKDQRIAVTLNKTAKHFSLHITETQPEDS | |
| (substituted) | AVYFCAASKSAIMVVLQTSSSLELALCLLSSQVNIQNPEPAVYQLKDPRSQD | |
| | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| α chain with | MASIRAVFIFLWLQLDLVNGENVEQHPSTLSVQEGDSAVIKCTYSDSASNYF | 1019 |
| alternative signal | PWYKQELGKGPQLIIDIRSNVGEKKDQRIAVTLNKTAKHFSLHITETQPEDS | |
| peptide, Cα | AVYFCAASKSAIMVVLQTSSSLELALCLLSSQVNIQNPEPAVYQLKDPRSQD | |
| (substituted) | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| α chain with | MHSIRAVFIFLWLQLDLVNGENVEQHPSTLSVQEGDSAVIKCTYSDSASNYF | 1020 |
| alternative signal | PWYKQELGKGPQLIIDIRSNVGEKKDQRIAVTLNKTAKHFSLHITETQPEDS | |
| peptide, Cα | AVYFCAASKSAIMVVLQTSSSLELALCLLSSQVNIQNPEPAVYQLKDPRSQD | |
| (substituted) | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| CDR1β | MNHEY | 2011 |
| CDR2β | SMNVEV | 2012 |
| CDR3β | ASSIQQGADTQY | 2013 |
| Vβ without signal | QVTQNPRYLITVTGKKLTVTCSQNMNHEYMSWYRQDPGLGLRQIYYSMNVEV | 2014 |
| peptide (SignalP) | TDKGDVPEGYKVSRKEKRNFPLILESPSPNQTSLYFCASSIQQGADTQYFGP | |
| | GTRLTVL | |
| Vβ without signal | EAQVTQNPRYLITVTGKKLTVTCSQNMNHEYMSWYRQDPGLGLRQIYYSMNV | 2015 |
| peptide (IMGT) | EVTDKGDVPEGYKVSRKEKRNFPLILESPSPNQTSLYFCASSIQQGADTQYF | |
| | GPGTRLTVL | |
| Vβ | MXPQLLGYVVLCLLGAGPLEAQVTQNPRYLITVTGKKLTVTCSQNMNHEYMS | 2016 |
| | WYRQDPGLGLRQIYYSMNVEVTDKGDVPEGYKVSRKEKRNFPLILESPSPNQ | |
| | TSLYFCASSIQQGADTQYFGPGTRLTVL | 2017 |
| | (X = any amino acid) | |
| β chain with WT signal | MGPQLLGYVVLCLLGAGPLEAQVTQNPRYLITVTGKKLTVTCSQNMNHEYMS | 2018 |
| peptide, Cβ | WYRQDPGLGLRQIYYSMNVEVTDKGDVPEGYKVSRKEKRNFPLILESPSPNQ | |
| (substituted) | TSLYFCASSIQQGADTQYFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MAPQLLGYVVLCLLGAGPLEAQVTQNPRYLITVTGKKLTVTCSQNMNHEYMS | 2019 |
| signal peptide, Cß | WYRQDPGLGLRQIYYSMNVEVTDKGDVPEGYKVSRKEKRNFPLILESPSPNQ | |
| (substituted) | TSLYFCASSIQQGADTQYFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MHPQLLGYVVLCLLGAGPLEAQVTQNPRYLITVTGKKLTVTCSQNMNHEYMS | 2020 |
| signal peptide, Cβ | WYRQDPGLGLRQIYYSMNVEVTDKGDVPEGYKVSRKEKRNFPLILESPSPNQ | |
| (substituted) | TSLYFCASSIQQGADTQYFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR002 interacts with and/or is specific for a peptide from p53. In some embodiments, the peptide is from a neoantigen of p53. In some embodiments, the neoantigen has the amino acid change R175H relative to the wild type p53 sequence. In some embodiments, TCR002 interacts with the neoantigen in the context of HLA-A*02:01, as described in International Publication No. WO 2019/067243, incorporated herein by reference in its entirety.

TABLE 6C
Amino acid sequences of TCR003.

| | | SEQ |
|---|---|---|
| | | ID |
| Description | Sequence | NO: |
| CDR1α | NSAFQY | 1021 |
| CDR2α | TYSSGN | 1022 |
| CDR3α | AMSGLKEDSSYKLI | 1023 |
| Vα w/o signal peptide | QQKEVEQDPGPLSVPEGAIVSLNCTYSNSAFQYFMWYRQYSRKGPELLMYTY | 1024 |
| (SignalP) | SSGNKEDGRFTAQVDKSSKYISLFIRDSQPSDSATYLCAMSGLKEDSSYKLI | |
| | FGSGTRLLVRP | |
| Vα w/o signal peptide | QKEVEQDPGPLSVPEGAIVSLNCTYSNSAFQYFMWYRQYSRKGPELLMYTYS | 1025 |
| (IMGT) | SGNKEDGRFTAQVDKSSKYISLFIRDSQPSDSATYLCAMSGLKEDSSYKLIF | |
| | GSGTRLLVRP | |
| Vα | MXKSLRVLLVILWLQLSWVWSQQKEVEQDPGPLSVPEGAIVSLNCTYSNSAF | 1026 |
| | QYFMWYRQYSRKGPELLMYTYSSGNKEDGRFTAQVDKSSKYISLFIRDSQPS | 1027 |
| | DSATYLCAMSGLKEDSSYKLIFGSGTRLLVRP | |
| | (X = any amino acid) | |
| α chain w/WT signal | MMKSLRVLLVILWLQLSWVWSQQKEVEQDPGPLSVPEGAIVSLNCTYSNSAF | 1028 |
| peptide, Cα | QYFMWYRQYSRKGPELLMYTYSSGNKEDGRFTAQVDKSSKYISLFIRDSQPS | |
| (substituted) | DSATYLCAMSGLKEDSSYKLIFGSGTRLLVRPNIQNPEPAVYQLKDPRSQDS | |
| | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| α chain w/alternative | MAKSLRVLLVILWLQLSWVWSQQKEVEQDPGPLSVPEGAIVSLNCTYSNSAF | 1029 |
| signal peptide, Cα | QYFMWYRQYSRKGPELLMYTYSSGNKEDGRFTAQVDKSSKYISLFIRDSQPS | |
| (substituted) | DSATYLCAMSGLKEDSSYKLIFGSGTRLLVRPNIQNPEPAVYQLKDPRSQDS | |
| | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| α chain w/alternative | MHKSLRVLLVILWLQLSWVWSQQKEVEQDPGPLSVPEGAIVSLNCTYSNSAF | 1030 |
| signal peptide, Cα | QYFMWYRQYSRKGPELLMYTYSSGNKEDGRFTAQVDKSSKYISLFIRDSQPS | |
| (substituted) | DSATYLCAMSGLKEDSSYKLIFGSGTRLLVRPNIQNPEPAVYQLKDPRSQDS | |
| | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| CDR1β | MNHEY | 2021 |
| CDR2β | SMNVEV | 2022 |
| CDR3β | ASSIQQGADTQY | 2023 |
| Vβ w/o signal peptide | QVTQNPRYLITVTGKKLTVTCSQNMNHEYMSWYRQDPGLGLRQIYYSMNVEV | 2024 |
| (SignalP) | TDKGDVPEGYKVSRKEKRNFPLILESPSPNQTSLYFCASSIQQGADTQYFGP | |
| | GTRLTVL | |
| Vβ w/o signal peptide | EAQVTQNPRYLITVTGKKLTVTCSQNMNHEYMSWYRQDPGLGLRQIYYSMNV | 2025 |
| (IMGT) | EVTDKGDVPEGYKVSRKEKRNFPLILESPSPNQTSLYFCASSIQQGADTQYF | |
| | GPGTRLTVL | |
| Vβ | MXPQLLGYVVLCLLGAGPLEAQVTQNPRYLITVTGKKLTVTCSQNMNHEYMS | 2026 |
| | WYRQDPGLGLRQIYYSMNVEVTDKGDVPEGYKVSRKEKRNFPLILESPSPNQ | 2027 |
| | TSLYFCASSIQQGADTQYFGPGTRLTVL | |
| | (X = any amino acid) | |
| β chain w/WT signal | MGPQLLGYVVLCLLGAGPLEAQVTQNPRYLITVTGKKLTVTCSQNMNHEYMS | 2028 |
| peptide, Cβ | WYRQDPGLGLRQIYYSMNVEVTDKGDVPEGYKVSRKEKRNFPLILESPSPNQ | |
| (substituted) | TSLYFCASSIQQGADTQYFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MAPQLLGYVVLCLLGAGPLEAQVTQNPRYLITVTGKKLTVTCSQNMNHEYMS | 2029 |
| signal peptide, Cβ | WYRQDPGLGLRQIYYSMNVEVTDKGDVPEGYKVSRKEKRNFPLILESPSPNQ | |
| (substituted) | TSLYFCASSIQQGADTQYFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MHPQLLGYVVLCLLGAGPLEAQVTQNPRYLITVTGKKLTVTCSQNMNHEYMS | 2030 |
| signal peptide, Cβ | WYRQDPGLGLRQIYYSMNVEVTDKGDVPEGYKVSRKEKRNFPLILESPSPNQ | |
| (substituted) | TSLYFCASSIQQGADTQYFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR003 interacts with and/or is specific for a peptide from p53. In some embodiments, the peptide is from a neoantigen of p53. In some embodiments, the neoantigen has the amino acid change R175H relative to the wild type p53 sequence. In some embodiments, TCR003 interacts with the neoantigen in the context of HLA-A*02:01, as described in International Publication No. WO 2020/264269, incorporated herein by reference in its entirety.

TABLE 6D
Amino acid sequences of TCR004.

| | | SEQ |
|---|---|---|
| | | ID |
| Description | Sequence | NO: |
| CDR1α | NSASQS | 1031 |
| CDR2α | VYSSGN | 1032 |
| CDR3α | VVQPGGYQKVT | 1033 |
| Vα w/o signal peptide | QRKEVEQDPGPFNVPEGATVAFNCTYSNSASQSFFWYRQDCRKEPKLLMSVY | 1034 |
| (SignalP) | SSGNEDGRFTAQLNRASQYISLLIRDSKLSDSATYLCVVQPGGYQKVTFGTG | |
| | TKLQVIP | |
| Vα w/o signal peptide | RKEVEQDPGPFNVPEGATVAFNCTYSNSASQSFFWYRQDCRKEPKLLMSVYS | 1035 |
| (IMGT) | SGNEDGRFTAQLNRASQYISLLIRDSKLSDSATYLCVVQPGGYQKVTFGTGT | |
| | KLQVIP | |
| Vα | MXSLRVLLVILWLQLSWVWSQRKEVEQDPGPFNVPEGATVAFNCTYSNSASQ | 1036 |
| | SFFWYRQDCRKEPKLLMSVYSSGNEDGRFTAQLNRASQYISLLIRDSKLSDS | 1037 |
| | ATYLCVVQPGGYQKVTFGTGTKLQVIP | |
| | (X = any amino acid) | |
| α chain w/WT signal | MISLRVLLVILWLQLSWVWSQRKEVEQDPGPFNVPEGATVAFNCTYSNSASQ | 1038 |
| peptide, Cα | SFFWYRQDCRKEPKLLMSVYSSGNEDGRFTAQLNRASQYISLLIRDSKLSDS | |
| (substituted) | ATYLCVVQPGGYQKVTFGTGTKLQVIPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain w/alternative | MASLRVLLVILWLQLSWVWSQRKEVEQDPGPFNVPEGATVAFNCTYSNSASQ | 1039 |
| signal peptide, Cα | SFFWYRQDCRKEPKLLMSVYSSGNEDGRFTAQLNRASQYISLLIRDSKLSDS | |
| (substituted) | ATYLCVVQPGGYQKVTFGTGTKLQVIPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain w/alternative | MHSLRVLLVILWLQLSWVWSQRKEVEQDPGPFNVPEGATVAFNCTYSNSASQ | 1040 |
| signal peptide, Cα | SFFWYRQDCRKEPKLLMSVYSSGNEDGRFTAQLNRASQYISLLIRDSKLSDS | |
| (substituted) | ATYLCVVQPGGYQKVTFGTGTKLQVIPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| CDR1β | MNHNS | 2031 |
| CDR2β | SASEGT | 2032 |
| CDR3β | ASSEGLWQVGDEQY | 2033 |
| Vβ w/o signal peptide | GVTQTPKFQVLKTGQSMTLQCAQDMNHNSMYWYRQDPGMGLRLIYYSASEGT | 2034 |
| (SignalP) | TDKGEVPNGYNVSRLNKREFSLRLESAAPSQTSVYFCASSEGLWQVGDEQYF | |
| | GPGTRLTVT | |
| Vβ w/o signal peptide | NAGVTQTPKFQVLKTGQSMTLQCAQDMNHNSMYWYRQDPGMGLRLIYYSASE | 2035 |
| (IMGT) | GTTDKGEVPNGYNVSRLNKREFSLRLESAAPSQTSVYFCASSEGLWQVGDEQ | |
| | YFGPGTRLTVT | |
| Vβ | MXIGLLCCVAFSLLWASPVNAGVTQTPKFQVLKTGQSMTLQCAQDMNHNSMY | 2036 |
| | WYRQDPGMGLRLIYYSASEGTTDKGEVPNGYNVSRLNKREFSLRLESAAPSQ | 2037 |
| | TSVYFCASSEGLWQVGDEQYFGPGTRLTVT | |
| | (X = any amino acid) | |
| β chain w/WT signal | MSIGLLCCVAFSLLWASPVNAGVTQTPKFQVLKTGQSMTLQCAQDMNHNSMY | 2038 |
| peptide, Cβ | WYRQDPGMGLRLIYYSASEGTTDKGEVPNGYNVSRLNKREFSLRLESAAPSQ | |
| (substituted) | TSVYFCASSEGLWQVGDEQYFGPGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain w/alternative | MAIGLLCCVAFSLLWASPVNAGVTQTPKFQVLKTGQSMTLQCAQDMNHNSMY | 2039 |
| signal peptide, Cβ | WYRQDPGMGLRLIYYSASEGTTDKGEVPNGYNVSRLNKREFSLRLESAAPSQ | |
| (substituted) | TSVYFCASSEGLWQVGDEQYFGPGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain w/alternative | MHIGLLCCVAFSLLWASPVNAGVTQTPKFQVLKTGQSMTLQCAQDMNHNSMY | 2040 |
| signal peptide, Cβ | WYRQDPGMGLRLIYYSASEGTTDKGEVPNGYNVSRLNKREFSLRLESAAPSQ | |
| (substituted) | TSVYFCASSEGLWQVGDEQYFGPGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR004 interacts with and/or is specific for p53. In some embodiments, the peptide is from a neoantigen of p53. In some embodiments, the neoantigen has the amino acid change R175H relative to the wild type p53 sequence. In some embodiments, TCR004 interacts with the neoantigen in the context of HLA-A*02:01, as described in International Publication No. WO 2019/067243, incorporated herein by reference in its entirety.

TABLE 6E
Amino acid sequences of TCR005.

| | | SEQ |
|---|---|---|
| | | ID |
| Description | Sequence | NO: |
| CDR1α | TSENNYY | 1041 |
| CDR2α | QEAYKQQN | 1042 |
| CDR3α | AFMGYSGAGSYQLT | 1043 |
| Vα w/o signal peptide | QTVTQSQPEMSVQEAETVTLSCTYDTSENNYYLFWYKQPPSRQMILVIRQEA | 1044 |
| (SignalP) | YKQQNATENRFSVNFQKAAKSFSLKISDSQLGDTAMYFCAFMGYSGAGSYQL | |
| | TFGKGTKLSVIP | |
| Vα w/o signal peptide | AQTVTQSQPEMSVQEAETVTLSCTYDTSENNYYLFWYKQPPSRQMILVIRQE | 1045 |
| (IMGT) | AYKQQNATENRFSVNFQKAAKSFSLKISDSQLGDTAMYFCAFMGYSGAGSYQ | |
| | LTFGKGTKLSVIP | |
| Vα | MXRVSLLWAVVVSTCLESGMAQTVTQSQPEMSVQEAETVTLSCTYDTSENNY | 1046 |
| | YLFWYKQPPSRQMILVIRQEAYKQQNATENRFSVNFQKAAKSFSLKISDSQL | 1047 |
| | GDTAMYFCAFMGYSGAGSYQLTFGKGTKLSVIP | |
| | (X = any amino acid) | |
| α chain w/WT signal | MTRVSLLWAVVVSTCLESGMAQTVTQSQPEMSVQEAETVTLSCTYDTSENNY | 1048 |
| peptide, Cα | YLFWYKQPPSRQMILVIRQEAYKQQNATENRFSVNFQKAAKSFSLKISDSQL | |
| (substituted) | GDTAMYFCAFMGYSGAGSYQLTFGKGTKLSVIPNIQNPEPAVYQLKDPRSQD | |
| | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| α chain w/alternative | MARVSLLWAVVVSTCLESGMAQTVTQSQPEMSVQEAETVTLSCTYDTSENNY | 1049 |
| signal peptide, Cα | YLFWYKQPPSRQMILVIRQEAYKQQNATENRFSVNFQKAAKSFSLKISDSQL | |
| (substituted) | GDTAMYFCAFMGYSGAGSYQLTFGKGTKLSVIPNIQNPEPAVYQLKDPRSQD | |
| | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| α chain w/alternative | MHRVSLLWAVVVSTCLESGMAQTVTQSQPEMSVQEAETVTLSCTYDTSENNY | 1050 |
| signal peptide, Cα | YLFWYKQPPSRQMILVIRQEAYKQQNATENRFSVNFQKAAKSFSLKISDSQL | |
| (substituted) | GDTAMYFCAFMGYSGAGSYQLTFGKGTKLSVIPNIQNPEPAVYQLKDPRSQD | |
| | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| CDR1β | ENHRY | 2041 |
| CDR2β | SYGVKD | 2042 |
| CDR3β | AISELVTGDSPLH | 2043 |
| Vβ w/o signal peptide | GITQSPRHKVTETGTPVTLRCHQTENHRYMYWYRQDPGHGLRLIHYSYGVKD | 2044 |
| (SignalP) | TDKGEVSDGYSVSRSKTEDFLLTLESATSSQTSVYFCAISELVTGDSPLHFG | |
| | NGTRLTVT | |
| Vβ w/o signal peptide | DAGITQSPRHKVTETGTPVTLRCHQTENHRYMYWYRQDPGHGLRLIHYSYGV | 2045 |
| (IMGT) | KDTDKGEVSDGYSVSRSKTEDFLLTLESATSSQTSVYFCAISELVTGDSPLH | |
| | FGNGTRLTVT | |
| Vβ | MXTRLFFYVALCLLWTGHMDAGITQSPRHKVTETGTPVTLRCHQTENHRYMY | 2046 |
| | WYRQDPGHGLRLIHYSYGVKDTDKGEVSDGYSVSRSKTEDFLLTLESATSSQ | 2047 |
| | TSVYFCAISELVTGDSPLHFGNGTRLTVT | |
| | (X = any amino acid) | |
| β chain w/WT signal | MGTRLFFYVALCLLWTGHMDAGITQSPRHKVTETGTPVTLRCHQTENHRYMY | 2048 |
| peptide, Cβ | WYRQDPGHGLRLIHYSYGVKDTDKGEVSDGYSVSRSKTEDFLLTLESATSSQ | |
| (substituted) | TSVYFCAISELVTGDSPLHFGNGTRLTVTEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/alternative | MATRLFFYVALCLLWTGHMDAGITQSPRHKVTETGTPVTLRCHQTENHRYMY | 2049 |
| signal peptide, Cβ | WYRQDPGHGLRLIHYSYGVKDTDKGEVSDGYSVSRSKTEDFLLTLESATSSQ | |
| (substituted) | TSVYFCAISELVTGDSPLHFGNGTRLTVTEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/alternative | MHTRLFFYVALCLLWTGHMDAGITQSPRHKVTETGTPVTLRCHQTENHRYMY | 2050 |
| signal peptide, Cβ | WYRQDPGHGLRLIHYSYGVKDTDKGEVSDGYSVSRSKTEDFLLTLESATSSQ | |
| (substituted) | TSVYFCAISELVTGDSPLHFGNGTRLTVTEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR005 interacts with and/or is specific for p53. In some embodiments, the peptide is from a neoantigen of p53. In some embodiments, the neoantigen has the amino acid change R175H relative to the wild type p53 sequence. In some embodiments, TCR005 interacts with the neoantigen in the context of HLA-A*02:01, as described in International Publication No. WO 2019/067243, incorporated herein by reference in its entirety.

TABLE 6F
Amino acid sequences of TCR006.

| | | SEQ |
|---|---|---|
| | | ID |
| Description | Sequence | NO: |
| CDR1α | TISGNEY | 1051 |
| CDR2α | GLKNN | 1052 |
| CDR3α | IVRGSPGAGGTSYGKLT | 1053 |
| Vα w/o signal peptide | KTTQPPSMDCAEGRAANLPCNHSTISGNEYVYWYRQIHSQGPQYIIHGLKNN | 1054 |
| (SignalP) | ETNEMASLIITEDRKSSTLILPHATLRDTAVYYCIVRGSPGAGGTSYGKLTF | |
| | GQGTILTVHP | |
| Vα w/o signal peptide | DAKTTQPPSMDCAEGRAANLPCNHSTISGNEYVYWYRQIHSQGPQYIIHGLK | 1055 |
| (IMGT) | NNETNEMASLIITEDRKSSTLILPHATLRDTAVYYCIVRGSPGAGGTSYGKL | |
| | TFGQGTILTVHP | |
| Vα | MXLVARVTVFLTFGTIIDAKTTQPPSMDCAEGRAANLPCNHSTISGNEYVYW | 1056 |
| | YRQIHSQGPQYIIHGLKNNETNEMASLIITEDRKSSTLILPHATLRDTAVYY | 1057 |
| | CIVRGSPGAGGTSYGKLTFGQGTILTVHP | |
| | (X = any amino acid) | |
| α chain w/WT signal | MRLVARVTVFLTFGTIIDAKTTQPPSMDCAEGRAANLPCNHSTISGNEYVYW | 1058 |
| peptide, Cα | YRQIHSQGPQYIIHGLKNNETNEMASLIITEDRKSSTLILPHATLRDTAVYY | |
| (substituted) | CIVRGSPGAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain w/alternative | MALVARVTVFLTFGTIIDAKTTQPPSMDCAEGRAANLPCNHSTISGNEYVYW | 1059 |
| signal peptide, Cα | YRQIHSQGPQYIIHGLKNNETNEMASLIITEDRKSSTLILPHATLRDTAVYY | |
| (substituted) | CIVRGSPGAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain w/alternative | MHLVARVTVFLTFGTIIDAKTTQPPSMDCAEGRAANLPCNHSTISGNEYVYW | 1060 |
| signal peptide, Cα | YRQIHSQGPQYIIHGLKNNETNEMASLIITEDRKSSTLILPHATLRDTAVYY | |
| (substituted) | CIVRGSPGAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| CDR1β | LNHDA | 2051 |
| CDR2β | SQIVND | 2052 |
| CDR3β | ASSIRTEAF | 2053 |
| Vβ w/o signal peptide | GITQSPKYLFRKEGQNVTLSCEQNLNHDAMYWYRQDPGQGLRLIYYSQIVND | 2054 |
| (SignalP) | FQKGDIAEGYSVSREKKESFPLTVTSAQKNPTAFYLCASSIRTEAFFGQGTR | |
| | LTVV | |
| Vβ w/o signal peptide | DGGITQSPKYLFRKEGQNVTLSCEQNLNHDAMYWYRQDPGQGLRLIYYSQIV | 2055 |
| (IMGT) | NDFQKGDIAEGYSVSREKKESFPLTVTSAQKNPTAFYLCASSIRTEAFFGQG | |
| | TRLTVV | |
| Vβ | MXNQVLCCVVLCFLGANTVDGGITQSPKYLFRKEGQNVTLSCEQNLNHDAMY | 2056 |
| | WYRQDPGQGLRLIYYSQIVNDFQKGDIAEGYSVSREKKESFPLTVTSAQKNP | 2057 |
| | TAFYLCASSIRTEAFFGQGTRLTVV | |
| | (X = any amino acid) | |
| β chain w/WT signal | MSNQVLCCVVLCFLGANTVDGGITQSPKYLFRKEGQNVTLSCEQNLNHDAMY | 2058 |
| peptide, Cβ | WYRQDPGQGLRLIYYSQIVNDFQKGDIAEGYSVSREKKESFPLTVTSAQKNP | |
| (substituted) | TAFYLCASSIRTEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| β chain w/alternative | MANQVLCCVVLCFLGANTVDGGITQSPKYLFRKEGQNVTLSCEQNLNHDAMY | 2059 |
| signal peptide, Cβ | WYRQDPGQGLRLIYYSQIVNDFQKGDIAEGYSVSREKKESFPLTVTSAQKNP | |
| (substituted) | TAFYLCASSIRTEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| β chain w/alternative | MHNQVLCCVVLCFLGANTVDGGITQSPKYLFRKEGQNVTLSCEQNLNHDAMY | 2060 |
| signal peptide, Cβ | WYRQDPGQGLRLIYYSQIVNDFQKGDIAEGYSVSREKKESFPLTVTSAQKNP | |
| (substituted) | TAFYLCASSIRTEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |

In some embodiments, TCR006 interacts with and/or is specific for p53. In some embodiments, the peptide is from a neoantigen of p53. In some embodiments, the neoantigen has the amino acid change R175H relative to the wild type p53 sequence. In some embodiments, TCR006 interacts with the neoantigen in the context of HLA-DRB1*13:01, as described in International Publication No. WO 2020/264269, incorporated herein by reference in its entirety.

TABLE 6G
Amino acid sequences of TCR007.

| | | SEQ |
|---|---|---|
| | | ID |
| Description | Sequence | NO: |
| CDR1α | TTSDR | 1061 |
| CDR2α | LLSNGAV | 1062 |
| CDR3α | AVAHMDSNYQLI | 1063 |
| Vα w/o signal peptide | ELKVEQNPLFLSMQEGKNYTIYCNYSTTSDRLYWYRQDPGKSLESLFVLLSN | 1064 |
| (SignalP) | GAVKQEGRLMASLDTKARLSTLHITAAVHDLSATYFCAVAHMDSNYQLIWGA | |
| | GTKLIIKP | |
| Vα w/o signal peptide | ELKVEQNPLFLSMQEGKNYTIYCNYSTTSDRLYWYRQDPGKSLESLFVLLSN | 1065 |
| (IMGT) | GAVKQEGRLMASLDTKARLSTLHITAAVHDLSATYFCAVAHMDSNYQLIWGA | |
| | GTKLIIKP | |
| Vα | MXKLLAMILWLQLDRLSGELKVEQNPLFLSMQEGKNYTIYCNYSTTSDRLYW | 1066 |
| | YRQDPGKSLESLFVLLSNGAVKQEGRLMASLDTKARLSTLHITAAVHDLSAT | 1067 |
| | YFCAVAHMDSNYQLIWGAGTKLIIKP | |
| | (X = any amino acid) | |
| α chain w/WT signal | MKKLLAMILWLQLDRLSGELKVEQNPLFLSMQEGKNYTIYCNYSTTSDRLYW | 1068 |
| peptide, Cα | YRQDPGKSLESLFVLLSNGAVKQEGRLMASLDTKARLSTLHITAAVHDLSAT | |
| (substituted) | YFCAVAHMDSNYQLIWGAGTKLIIKPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain w/alternative | MAKLLAMILWLQLDRLSGELKVEQNPLFLSMQEGKNYTIYCNYSTTSDRLYW | 1069 |
| signal peptide, Cα | YRQDPGKSLESLFVLLSNGAVKQEGRLMASLDTKARLSTLHITAAVHDLSAT | |
| (substituted) | YFCAVAHMDSNYQLIWGAGTKLIIKPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain w/alternative | MHKLLAMILWLQLDRLSGELKVEQNPLFLSMQEGKNYTIYCNYSTTSDRLYW | 1070 |
| signal peptide, Cα | YRQDPGKSLESLFVLLSNGAVKQEGRLMASLDTKARLSTLHITAAVHDLSAT | |
| (substituted) | YFCAVAHMDSNYQLIWGAGTKLIIKPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| CDR1β | MNHEY | 2061 |
| CDR2β | SVGEGT | 2062 |
| CDR3β | ASSYAGLAAPREQF | 2063 |
| Vβ w/o signal peptide | GVTQTPKFRVLKTGQSMTLLCAQDMNHEYMYWYRQDPGMGLRLIHYSVGEGT | 2064 |
| (SignalP) | TAKGEVPDGYNVSRLKKQNFLLGLESAAPSQTSVYFCASSYAGLAAPREQFF | |
| | GPGTRLTVL | |
| Vβ w/o signal peptide | NAGVTQTPKFRVLKTGQSMTLLCAQDMNHEYMYWYRQDPGMGLRLIHYSVGE | 2065 |
| (IMGT) | GTTAKGEVPDGYNVSRLKKQNFLLGLESAAPSQTSVYFCASSYAGLAAPREQ | |
| | FFGPGTRLTVL | |
| Vβ | MXLGLLCCGAFSLLWAGPVNAGVTQTPKFRVLKTGQSMTLLCAQDMNHEYMY | 2066 |
| | WYRQDPGMGLRLIHYSVGEGTTAKGEVPDGYNVSRLKKQNFLLGLESAAPSQ | 2067 |
| | TSVYFCASSYAGLAAPREQFFGPGTRLTVL | |
| | (X = any amino acid) | |
| β chain w/WT signal | MSLGLLCCGAFSLLWAGPVNAGVTQTPKFRVLKTGQSMTLLCAQDMNHEYMY | 2068 |
| peptide, Cβ | WYRQDPGMGLRLIHYSVGEGTTAKGEVPDGYNVSRLKKQNFLLGLESAAPSQ | |
| (substituted) | TSVYFCASSYAGLAAPREQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain w/alternative | MALGLLCCGAFSLLWAGPVNAGVTQTPKFRVLKTGQSMTLLCAQDMNHEYMY | 2069 |
| signal peptide, Cβ | WYRQDPGMGLRLIHYSVGEGTTAKGEVPDGYNVSRLKKQNFLLGLESAAPSQ | |
| (substituted) | TSVYFCASSYAGLAAPREQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain w/alternative | MHLGLLCCGAFSLLWAGPVNAGVTQTPKFRVLKTGQSMTLLCAQDMNHEYMY | 2070 |
| signal peptide, Cβ | WYRQDPGMGLRLIHYSVGEGTTAKGEVPDGYNVSRLKKQNFLLGLESAAPSQ | |
| (substituted) | TSVYFCASSYAGLAAPREQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR007 interacts with and/or is specific for p53. In some embodiments, the peptide is from a neoantigen of p53. In some embodiments, the neoantigen has the amino acid change R175H relative to the wild type p53 sequence. In some embodiments, TCR007 interacts with the neoantigen in the context of HLA-DRB1*13:01, as described in International Publication No. WO 2020/264269, incorporated herein by reference in its entirety.

TABLE 6H
Amino acid sequences of TCR008.

| | | SEQ |
|---|---|---|
| | | ID |
| Description | Sequence | NO: |
| CDR1α | TISGNEY | 1071 |
| CDR2α | GLKNN | 1072 |
| CDR3α | IVRARANAGGTSYGKLT | 1073 |
| Vα w/o signal peptide | KTTQPPSMDCAEGRAANLPCNHSTISGNEYVYWYRQIHSQGPQYIIHGLKNN | 1074 |
| (SignalP) | ETNEMASLIITEDRKSSTLILPHATLRDTAVYYCIVRARANAGGTSYGKLTF | |
| | GQGTILTVHP | |
| Vα w/o signal peptide | DAKTTQPPSMDCAEGRAANLPCNHSTISGNEYVYWYRQIHSQGPQYIIHGLK | 1075 |
| (IMGT) | NNETNEMASLIITEDRKSSTLILPHATLRDTAVYYCIVRARANAGGTSYGKL | |
| | TFGQGTILTVHP | |
| Vα | MXLVARVTVFLTFGTIIDAKTTQPPSMDCAEGRAANLPCNHSTISGNEYVYW | 1076 |
| | YRQIHSQGPQYIIHGLKNNETNEMASLIITEDRKSSTLILPHATLRDTAVYY | 1077 |
| | CIVRARANAGGTSYGKLTFGQGTILTVHP | |
| | (X = any amino acid) | |
| α chain w/WT signal | MRLVARVTVFLTFGTIIDAKTTQPPSMDCAEGRAANLPCNHSTISGNEYVYW | 1078 |
| peptide, Cα | YRQIHSQGPQYIIHGLKNNETNEMASLIITEDRKSSTLILPHATLRDTAVYY | |
| (substituted) | CIVRARANAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain w/alternative | MALVARVTVFLTFGTIIDAKTTQPPSMDCAEGRAANLPCNHSTISGNEYVYW | 1079 |
| signal peptide, Cα | YRQIHSQGPQYIIHGLKNNETNEMASLIITEDRKSSTLILPHATLRDTAVYY | |
| (substituted) | CIVRARANAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain w/alternative | MHLVARVTVFLTFGTIIDAKTTQPPSMDCAEGRAANLPCNHSTISGNEYVYW | 1080 |
| signal peptide, Cα | YRQIHSQGPQYIIHGLKNNETNEMASLIITEDRKSSTLILPHATLRDTAVYY | |
| (substituted) | CIVRARANAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| CDR1β | LNHDA | 2071 |
| CDR2β | SQIVND | 2072 |
| CDR3β | ASLQFNEQF | 2073 |
| Vβ w/o signal peptide | GITQSPKYLFRKEGQNVTLSCEQNLNHDAMYWYRQDPGQGLRLIYYSQIVND | 2074 |
| (SignalP) | FQKGDIAEGYSVSREKKESFPLTVTSAQKNPTAFYLCASLQFNEQFFGPGTR | |
| | LTVL | |
| Vβ w/o signal peptide | DGGITQSPKYLFRKEGQNVTLSCEQNLNHDAMYWYRQDPGQGLRLIYYSQIV | 2075 |
| (IMGT) | NDFQKGDIAEGYSVSREKKESFPLTVTSAQKNPTAFYLCASLQFNEQFFGPG | |
| | TRLTVL | |
| Vβ | MXMSNQVLCCVVLCFLGANTVDGGITQSPKYLFRKEGQNVTLSCEQNLNHDA | 2076 |
| | MYWYRQDPGQGLRLIYYSQIVNDFQKGDIAEGYSVSREKKESFPLTVTSAQK | |
| | NPTAFYLCASLQFNEQFFGPGTRLTVL | |
| | (X = any amino acid) | |
| Vβ (alternative) | MXNQVLCCVVLCFLGANTVDGGITQSPKYLFRKEGQNVTLSCEQNLNHDAMY | 2077 |
| | WYRQDPGQGLRLIYYSQIVNDFQKGDIAEGYSVSREKKESFPLTVTSAQKNP | |
| | TAFYLCASLQFNEQFFGPGTRLTVL | |
| | (X = any amino acid) | |
| β chain w/WT signal | MSMSNQVLCCVVLCFLGANTVDGGITQSPKYLFRKEGQNVTLSCEQNLNHDA | 2078 |
| peptide, Cβ | MYWYRQDPGQGLRLIYYSQIVNDFQKGDIAEGYSVSREKKESFPLTVTSAQK | |
| (substituted) | NPTAFYLCASLQFNEQFFGPGTRLTVLEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain w/alternative | MAMSNQVLCCVVLCFLGANTVDGGITQSPKYLFRKEGQNVTLSCEQNLNHDA | 2079 |
| signal peptide, Cβ | MYWYRQDPGQGLRLIYYSQIVNDFQKGDIAEGYSVSREKKESFPLTVTSAQK | |
| (substituted) | NPTAFYLCASLQFNEQFFGPGTRLTVLEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain w/alternative | MHMSNQVLCCVVLCFLGANTVDGGITQSPKYLFRKEGQNVTLSCEQNLNHDA | 2080 |
| signal peptide, Cβ | MYWYRQDPGQGLRLIYYSQIVNDFQKGDIAEGYSVSREKKESFPLTVTSAQK | |
| (substituted) | NPTAFYLCASLQFNEQFFGPGTRLTVLEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |

In some embodiments, TCR008 interacts with and/or is specific for p53. In some embodiments, the peptide is from a neoantigen of p53. In some embodiments, the neoantigen has the amino acid change R175H relative to the wild type p53 sequence. In some embodiments, TCR008 interacts with the neoantigen in the context of HLA-DRB1*13:01, as described in International Publication No. WO 2020/264269, incorporated herein by reference in its entirety.

TABLE 6I
Amino acid sequences of TCR009.

| | | SEQ |
|---|---|---|
| | | ID |
| Description | Sequence | NO: |
| CDR1α | SSNFYA | 1081 |
| CDR2α | MTLNGDE | 1082 |
| CDR3α | ALITGGGNKLT | 1083 |
| Vα w/o signal peptide | ILNVEQSPQSLHVQEGDSTNFTCSFPSSNFYALHWYRWETAKSPEALFVMTL | 1084 |
| (SignalP) | NGDEKKKGRISATLNTKEGYSYLYIKGSQPEDSATYLCALITGGGNKLTFGT | |
| | GTQLKVEL | |
| Vα w/o signal peptide | ILNVEQSPQSLHVQEGDSTNFTCSFPSSNFYALHWYRWETAKSPEALFVMTL | 1085 |
| (IMGT) | NGDEKKKGRISATLNTKEGYSYLYIKGSQPEDSATYLCALITGGGNKLTFGT | |
| | GTQLKVEL | |
| Vα | MXKNPLAAPLLILWFHLDCVSSILNVEQSPQSLHVQEGDSTNFTCSFPSSNF | 1086 |
| | YALHWYRWETAKSPEALFVMTLNGDEKKKGRISATLNTKEGYSYLYIKGSQP | 1087 |
| | EDSATYLCALITGGGNKLTFGTGTQLKVEL | |
| | (X = any amino acid) | |
| α chain w/WT signal | MEKNPLAAPLLILWFHLDCVSSILNVEQSPQSLHVQEGDSTNFTCSFPSSNF | 1088 |
| peptide, Cα | YALHWYRWETAKSPEALFVMTLNGDEKKKGRISATLNTKEGYSYLYIKGSQP | |
| (substituted) | EDSATYLCALITGGGNKLTFGTGTQLKVELNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain w/alternative | MAKNPLAAPLLILWFHLDCVSSILNVEQSPQSLHVQEGDSTNFTCSFPSSNF | 1089 |
| signal peptide, Cα | YALHWYRWETAKSPEALFVMTLNGDEKKKGRISATLNTKEGYSYLYIKGSQP | |
| (substituted) | EDSATYLCALITGGGNKLTFGTGTQLKVELNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain w/alternative | MHKNPLAAPLLILWFHLDCVSSILNVEQSPQSLHVQEGDSTNFTCSFPSSNF | 1090 |
| signal peptide, Cα | YALHWYRWETAKSPEALFVMTLNGDEKKKGRISATLNTKEGYSYLYIKGSQP | |
| (substituted) | EDSATYLCALITGGGNKLTFGTGTQLKVELNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| CDR1β | MNHEY | 2081 |
| CDR2β | SVGEGT | 2082 |
| CDR3β | ASRLQGWNSPLH | 2083 |
| Vβ w/o signal peptide | GVTQTPKFRVLKTGQSMTLLCAQDMNHEYMYWYRQDPGMGLRLIHYSVGEGT | 2084 |
| (SignalP) | TAKGEVPDGYNVSRLKKQNFLLGLESAAPSQTSVYFCASRLQGWNSPLHFGN | |
| | GTRLTVT | |
| Vβ w/o signal peptide | NAGVTQTPKFRVLKTGQSMTLLCAQDMNHEYMYWYRQDPGMGLRLIHYSVGE | 2085 |
| (IMGT) | GTTAKGEVPDGYNVSRLKKQNFLLGLESAAPSQTSVYFCASRLQGWNSPLHF | |
| | GNGTRLTVT | |
| Vβ | MXLGLLCCGAFSLLWAGPVNAGVTQTPKFRVLKTGQSMTLLCAQDMNHEYMY | 2086 |
| | WYRQDPGMGLRLIHYSVGEGTTAKGEVPDGYNVSRLKKQNFLLGLESAAPSQ | 2087 |
| | TSVYFCASRLQGWNSPLHFGNGTRLTVT | |
| | (X = any amino acid) | |
| β chain w/WT signal | MSLGLLCCGAFSLLWAGPVNAGVTQTPKFRVLKTGQSMTLLCAQDMNHEYMY | 2088 |
| peptide, Cβ | WYRQDPGMGLRLIHYSVGEGTTAKGEVPDGYNVSRLKKQNFLLGLESAAPSQ | |
| (substituted) | TSVYFCASRLQGWNSPLHFGNGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MALGLLCCGAFSLLWAGPVNAGVTQTPKFRVLKTGQSMTLLCAQDMNHEYMY | 2089 |
| signal peptide, Cβ | WYRQDPGMGLRLIHYSVGEGTTAKGEVPDGYNVSRLKKQNFLLGLESAAPSQ | |
| (substituted) | TSVYFCASRLQGWNSPLHFGNGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MHLGLLCCGAFSLLWAGPVNAGVTQTPKFRVLKTGQSMTLLCAQDMNHEYMY | 2090 |
| signal peptide, Cβ | WYRQDPGMGLRLIHYSVGEGTTAKGEVPDGYNVSRLKKQNFLLGLESAAPSQ | |
| (substituted) | TSVYFCASRLQGWNSPLHFGNGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR009 interacts with and/or is specific for p53. In some embodiments, the peptide is from a neoantigen of p53. In some embodiments, the neoantigen has the amino acid change R175H relative to the wild type p53 sequence. In some embodiments, TCR009 interacts with the neoantigen in the context of HLA-DRB1*13:01, as described in International Publication No. WO 2019/067243, incorporated herein by reference in its entirety.

TABLE 6J
Amino acid sequences of TCR010.

| | | SEQ |
|---|---|---|
| | | ID |
| Description | Sequence | NO: |
| CDR1α | TTLSN | 1091 |
| CDR2α | LVKSGEV | 1092 |
| CDR3α | AGPGGAGSYQLT | 1093 |
| Vα w/o signal peptide | QQVMQIPQYQHVQEGEDFTTYCNSSTTLSNIQWYKQRPGGHPVFLIQLVKSG | 1094 |
| (SignalP) | EVKKQKRLTFQFGEAKKNSSLHITATQTTDVGTYFCAGPGGAGSYQLTFGKG | |
| | TKLSVIP | |
| Vα w/o signal peptide | GQQVMQIPQYQHVQEGEDFTTYCNSSTTLSNIQWYKQRPGGHPVFLIQLVKS | 1095 |
| (IMGT) | GEVKKQKRLTFQFGEAKKNSSLHITATQTTDVGTYFCAGPGGAGSYQLTFGK | |
| | GTKLSVIP | |
| Vα | MXLITSMLVLWMQLSQVNGQQVMQIPQYQHVQEGEDFTTYCNSSTTLSNIQW | 1096 |
| | YKQRPGGHPVFLIQLVKSGEVKKQKRLTFQFGEAKKNSSLHITATQTTDVGT | 1097 |
| | YFCAGPGGAGSYQLTFGKGTKLSVIP | |
| | (X = any amino acid) | |
| α chain w/WT signal | MLLITSMLVLWMQLSQVNGQQVMQIPQYQHVQEGEDFTTYCNSSTTLSNIQW | 1098 |
| peptide, Cα | YKQRPGGHPVFLIQLVKSGEVKKQKRLTFQFGEAKKNSSLHITATQTTDVGT | |
| (substituted) | YFCAGPGGAGSYQLTFGKGTKLSVIPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain w/alternative | MALITSMLVLWMQLSQVNGQQVMQIPQYQHVQEGEDETTYCNSSTTLSNIQW | 1099 |
| signal peptide, Cα | YKQRPGGHPVFLIQLVKSGEVKKQKRLTFQFGEAKKNSSLHITATQTTDVGT | |
| (substituted) | YFCAGPGGAGSYQLTFGKGTKLSVIPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain w/alternative | MHLITSMLVLWMQLSQVNGQQVMQIPQYQHVQEGEDFTTYCNSSTTLSNIQW | 1100 |
| signal peptide, Cα | YKQRPGGHPVFLIQLVKSGEVKKQKRLTFQFGEAKKNSSLHITATQTTDVGT | |
| (substituted) | YFCAGPGGAGSYQLTFGKGTKLSVIPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| CDR1β | MNHEY | 2091 |
| CDR2β | SMNVEV | 2092 |
| CDR3β | ASSPFVVIGQINEQY | 2093 |
| Vβ w/o signal peptide | QVTQNPRYLITVTGKKLTVTCSQNMNHEYMSWYRQDPGLGLRQIYYSMNVEV | 2094 |
| (SignalP) | TDKGDVPEGYKVSRKEKRNFPLILESPSPNQTSLYFCASSPFVVIGQINEQY | |
| | FGPGTRLTVT | |
| Vβ w/o signal peptide | EAQVTQNPRYLITVTGKKLTVTCSQNMNHEYMSWYRQDPGLGLRQIYYSMNV | 2095 |
| (IMGT) | EVTDKGDVPEGYKVSRKEKRNFPLILESPSPNQTSLYFCASSPFVVIGQINE | |
| | QYFGPGTRLTVT | |
| Vβ | MXPQLLGYVVLCLLGAGPLEAQVTQNPRYLITVTGKKLTVTCSQNMNHEYMS | 2096 |
| | WYRQDPGLGLRQIYYSMNVEVTDKGDVPEGYKVSRKEKRNFPLILESPSPNQ | 2097 |
| | TSLYFCASSPFVVIGQINEQYFGPGTRLTVT | |
| | (X = any amino acid) | |
| β chain w/WT signal | MGPQLLGYVVLCLLGAGPLEAQVTQNPRYLITVTGKKLTVTCSQNMNHEYMS | 2098 |
| peptide, Cβ | WYRQDPGLGLRQIYYSMNVEVTDKGDVPEGYKVSRKEKRNFPLILESPSPNQ | |
| (substituted) | TSLYFCASSPFVVIGQINEQYFGPGTRLTVTEDLRNVTPPKVSLFEPSKAEI | |
| | ANKQKATLVCLARGFFPDHVELSWWVNGKEVHSGVCTDPQAYKESNYSYCLS | |
| | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |
| β chain w/alternative | MAPQLLGYVVLCLLGAGPLEAQVTQNPRYLITVTGKKLTVTCSQNMNHEYMS | 2099 |
| signal peptide, Cβ | WYRQDPGLGLRQIYYSMNVEVTDKGDVPEGYKVSRKEKRNFPLILESPSPNQ | |
| (substituted) | TSLYFCASSPFVVIGQINEQYFGPGTRLTVTEDLRNVTPPKVSLFEPSKAEI | |
| | ANKQKATLVCLARGFFPDHVELSWWVNGKEVHSGVCTDPQAYKESNYSYCLS | |
| | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |
| β chain w/alternative | MHPQLLGYVVLCLLGAGPLEAQVTQNPRYLITVTGKKLTVTCSQNMNHEYMS | 2100 |
| signal peptide, Cβ | WYRQDPGLGLRQIYYSMNVEVTDKGDVPEGYKVSRKEKRNFPLILESPSPNQ | |
| (substituted) | TSLYFCASSPFVVIGQINEQYFGPGTRLTVTEDLRNVTPPKVSLFEPSKAEI | |
| | ANKQKATLVCLARGFFPDHVELSWWVNGKEVHSGVCTDPQAYKESNYSYCLS | |
| | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |

In some embodiments, TCR010 interacts with and/or is specific for p53. In some embodiments, the peptide is from a neoantigen of p53. In some embodiments, the neoantigen has the amino acid change R175H relative to the wild type p53 sequence. In some embodiments, TCR010 interacts with the neoantigen in the context of HLA-DRB1*13:01, as described in International Publication No. WO 2020/264269, incorporated herein by reference in its entirety.

TABLE 6K
Amino acid sequences of TCR011.

| | | SEQ |
|---|---|---|
| | | ID |
| Description | Sequence | NO: |
| CDR1α | TISGNEY | 1101 |
| CDR2α | GLKNN | 1102 |
| CDR3α | IVRARANAGGTSYGKLT | 1103 |
| Vα w/o signal peptide | KTTQPPSMDCAEGRAANLPCNHSTISGNEYVYWYRQIHSQGPQYIIHGLKNN | 1104 |
| (SignalP) | ETNEMASLIITEDRKSSTLILPHATLRDTAVYYCIVRARANAGGTSYGKLTF | |
| | GQGTILTVHP | |
| Vα w/o signal peptide | DAKTTQPPSMDCAEGRAANLPCNHSTISGNEYVYWYRQIHSQGPQYIIHGLK | 1105 |
| (IMGT) | NNETNEMASLIITEDRKSSTLILPHATLRDTAVYYCIVRARANAGGTSYGKL | |
| | TFGQGTILTVHP | |
| Vα | MXLVARVTVFLTFGTIIDAKTTQPPSMDCAEGRAANLPCNHSTISGNEYVYW | 1106 |
| | YRQIHSQGPQYIIHGLKNNETNEMASLIITEDRKSSTLILPHATLRDTAVYY | 1107 |
| | CIVRARANAGGTSYGKLTFGQGTILTVHP | |
| | (X = any amino acid) | |
| α chain w/WT signal | MRLVARVTVFLTFGTIIDAKTTQPPSMDCAEGRAANLPCNHSTISGNEYVYW | 1108 |
| peptide, Cα | YRQIHSQGPQYIIHGLKNNETNEMASLIITEDRKSSTLILPHATLRDTAVYY | |
| (substituted) | CIVRARANAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain w/alternative | MALVARVTVFLTFGTIIDAKTTQPPSMDCAEGRAANLPCNHSTISGNEYVYW | 1109 |
| signal peptide, Cα | YRQIHSQGPQYIIHGLKNNETNEMASLIITEDRKSSTLILPHATLRDTAVYY | |
| (substituted) | CIVRARANAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain w/alternative | MHLVARVTVFLTFGTIIDAKTTQPPSMDCAEGRAANLPCNHSTISGNEYVYW | 1110 |
| signal peptide, Cα | YRQIHSQGPQYIIHGLKNNETNEMASLIITEDRKSSTLILPHATLRDTAVYY | |
| (substituted) | CIVRARANAGGTSYGKLTFGQGTILTVHPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| CDR1β | LNHDA | 2101 |
| CDR2β | SQIVND | 2102 |
| CDR3β | ATRTGNEAF | 2103 |
| Vβ w/o signal peptide | GITQSPKYLFRKEGQNVTLSCEQNLNHDAMYWYRQDPGQGLRLIYYSQIVND | 2104 |
| (SignalP) | FQKGDIAEGYSVSREKKESFPLTVTSAQKNPTAFYLCATRTGNEAFFGQGTR | |
| | LTVV | |
| Vβ w/o signal peptide | DGGITQSPKYLFRKEGQNVTLSCEQNLNHDAMYWYRQDPGQGLRLIYYSQIV | 2105 |
| (IMGT) | NDFQKGDIAEGYSVSREKKESFPLTVTSAQKNPTAFYLCATRTGNEAFFGQG | |
| | TRLTVV | |
| Vβ | MXNQVLCCVVLCFLGANTVDGGITQSPKYLFRKEGQNVTLSCEQNLNHDAMY | 2106 |
| | WYRQDPGQGLRLIYYSQIVNDFQKGDIAEGYSVSREKKESFPLTVTSAQKNP | 2107 |
| | TAFYLCATRTGNEAFFGQGTRLTVV | |
| | (X = any amino acid) | |
| β chain w/WT signal | MSNQVLCCVVLCFLGANTVDGGITQSPKYLFRKEGQNVTLSCEQNLNHDAMY | 2108 |
| peptide, Cβ | WYRQDPGQGLRLIYYSQIVNDFQKGDIAEGYSVSREKKESFPLTVTSAQKNP | |
| (substituted) | TAFYLCATRTGNEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| β chain w/alternative | MANQVLCCVVLCFLGANTVDGGITQSPKYLFRKEGQNVTLSCEQNLNHDAMY | 2109 |
| signal peptide, Cβ | WYRQDPGQGLRLIYYSQIVNDFQKGDIAEGYSVSREKKESFPLTVTSAQKNP | |
| (substituted) | TAFYLCATRTGNEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| β chain w/alternative | MHNQVLCCVVLCFLGANTVDGGITQSPKYLFRKEGQNVTLSCEQNLNHDAMY | 2110 |
| signal peptide, Cβ | WYRQDPGQGLRLIYYSQIVNDFQKGDIAEGYSVSREKKESFPLTVTSAQKNP | |
| (substituted) | TAFYLCATRTGNEAFFGQGTRLTVVEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |

In some embodiments, TCR011 interacts with and/or is specific for p53. In some embodiments, the peptide is from a neoantigen of p53. In some embodiments, the neoantigen has the amino acid change R175H relative to the wild type p53 sequence. In some embodiments, TCR011 interacts with the neoantigen in the context of HLA-DRB1*13:01, as described in International Publication No. WO 2020/264269, incorporated herein by reference in its entirety.

TABLE 6L
Amino acid sequences of TCR012.

| | | SEQ |
|---|---|---|
| | | ID |
| Description | Sequence | NO: |
| CDR1α | VSNAYN | 1111 |
| CDR2α | GSKP | 1112 |
| CDR3α | AVEDRRRTALI | 1113 |
| Vα w/o signal peptide | KDQVFQPSTVASSEGAVVEIFCNHSVSNAYNFFWYLHFPGCAPRLLVKGSKP | 1114 |
| (SignalP) | SQQGRYNMTYERFSSSLLILQVREADAAVYYCAVEDRRRTALIFGKGTTLSV | |
| | SS | |
| Vα w/o signal peptide | KDQVFQPSTVASSEGAVVEIFCNHSVSNAYNFFWYLHFPGCAPRLLVKGSKP | 1115 |
| (IMGT) | SQQGRYNMTYERFSSSLLILQVREADAAVYYCAVEDRRRTALIFGKGTTLSV | |
| | SS | |
| Vα | MXLQSTLGAVWLGLLLNSLWKVAESKDQVFQPSTVASSEGAVVEIFCNHSVS | 1116 |
| | NAYNFFWYLHFPGCAPRLLVKGSKPSQQGRYNMTYERFSSSLLILQVREADA | 1117 |
| | AVYYCAVEDRRRTALIFGKGTTLSVSS | |
| | (X = any amino acid) | |
| α chain w/WT signal | MALQSTLGAVWLGLLLNSLWKVAESKDQVFQPSTVASSEGAVVEIFCNHSVS | 1118 |
| peptide, Cα | NAYNFFWYLHFPGCAPRLLVKGSKPSQQGRYNMTYERFSSSLLILQVREADA | 1119 |
| (substituted) | AVYYCAVEDRRRTALIFGKGTTLSVSSNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain w/alternative | MHLQSTLGAVWLGLLLNSLWKVAESKDQVFQPSTVASSEGAVVEIFCNHSVS | 1120 |
| signal peptide, Cα | NAYNFFWYLHFPGCAPRLLVKGSKPSQQGRYNMTYERFSSSLLILQVREADA | |
| (substituted) | AVYYCAVEDRRRTALIFGKGTTLSVSSNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| CDR1β | SNHLY | 2111 |
| CDR2β | FYNNEI | 2112 |
| CDR3β | ASSEYQSQSNEQF | 2113 |
| Vβ w/o signal peptide | EPEVTQTPSHQVTQMGQEVILRCVPISNHLYFYWYRQILGQKVEFLVSFYNN | 2114 |
| (SignalP) | EISEKSEIFDDQFSVERPDGSNFTLKIRSTKLEDSAMYFCASSEYQSQSNEQ | |
| | FFGPGTRLTVL | |
| Vβ w/o signal peptide | EPEVTQTPSHQVTQMGQEVILRCVPISNHLYFYWYRQILGQKVEFLVSFYNN | 2115 |
| (IMGT) | EISEKSEIFDDQFSVERPDGSNFTLKIRSTKLEDSAMYFCASSEYQSQSNEQ | |
| | FFGPGTRLTVL | |
| Vβ | MXTWLVCWAIFSLLKAGLTEPEVTQTPSHQVTQMGQEVILRCVPISNHLYFY | 2116 |
| | WYRQILGQKVEFLVSFYNNEISEKSEIFDDQFSVERPDGSNFTLKIRSTKLE | 2117 |
| | DSAMYFCASSEYQSQSNEQFFGPGTRLTVL | |
| | (X = any amino acid) | |
| β chain w/WT signal | MDTWLVCWAIFSLLKAGLTEPEVTQTPSHQVTQMGQEVILRCVPISNHLYFY | 2118 |
| peptide, Cβ | WYRQILGQKVEFLVSFYNNEISEKSEIFDDQFSVERPDGSNFTLKIRSTKLE | |
| (substituted) | DSAMYFCASSEYQSQSNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain w/alternative | MATWLVCWAIFSLLKAGLTEPEVTQTPSHQVTQMGQEVILRCVPISNHLYFY | 2119 |
| signal peptide, Cβ | WYRQILGQKVEFLVSFYNNEISEKSEIFDDQFSVERPDGSNFTLKIRSTKLE | |
| (substituted) | DSAMYFCASSEYQSQSNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain w/alternative | MHTWLVCWAIFSLLKAGLTEPEVTQTPSHQVTQMGQEVILRCVPISNHLYFY | 2120 |
| signal peptide, Cβ | WYRQILGQKVEFLVSFYNNEISEKSEIFDDQFSVERPDGSNFTLKIRSTKLE | |
| (substituted) | DSAMYFCASSEYQSQSNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR012 interacts with and/or is specific for p53. In some embodiments, the peptide is from a neoantigen of p53. In some embodiments, the neoantigen has the amino acid change R175H relative to the wild type p53 sequence. In some embodiments, TCR012 interacts with the neoantigen in the context of HLA-DRB1*13:01, as described in International Publication No. WO 2020/264269, incorporated herein by reference in its entirety.

TABLE 6M
Amino acid sequences of TCR013.

| | | SEQ |
|---|---|---|
| | | ID |
| Description | Sequence | NO: |
| CDR1α | TISGTDY | 1121 |
| CDR2α | GLTSN | 1122 |
| CDR3α | ILRDNNARLM | 1123 |
| Vα w/o signal peptide | KTTQPNSMESNEEEPVHLPCNHSTISGTDYIHWYRQLPSQGPEYVIHGLTSN | 1124 |
| (SignalP) | VNNRMASLAIAEDRKSSTLILHRATLRDAAVYYCILRDNNARLMFGDGTQLV | |
| | VKP | |
| Vα w/o signal peptide | DAKTTQPNSMESNEEEPVHLPCNHSTISGTDYIHWYRQLPSQGPEYVIHGLT | 1125 |
| (IMGT) | SNVNNRMASLAIAEDRKSSTLILHRATLRDAAVYYCILRDNNARLMFGDGTQ | |
| | LVVKP | |
| Vα | MXLVTSITVLLSLGIMGDAKTTQPNSMESNEEEPVHLPCNHSTISGTDYIHW | 1126 |
| | YRQLPSQGPEYVIHGLTSNVNNRMASLAIAEDRKSSTLILHRATLRDAAVYY | 1127 |
| | CILRDNNARLMFGDGTQLVVKP | |
| | (X = any amino acid) | |
| α chain w/WT signal | MKLVTSITVLLSLGIMGDAKTTQPNSMESNEEEPVHLPCNHSTISGTDYIHW | 1128 |
| peptide, Cα | YRQLPSQGPEYVIHGLTSNVNNRMASLAIAEDRKSSTLILHRATLRDAAVYY | |
| (substituted) | CILRDNNARLMFGDGTQLVVKPNIQNPEPAVYQLKDPRSQDSTLCLFTDFDS | |
| | QINVPKTMESGTFITDKCVLDMKAMDSKSNGAIAWSNQTSFTCQDIFKETNA | |
| | TYPSSDVPCDATLTEKSFETDMNLNFQNLLVIVLRILLLKVAGFNLLMTLRL | |
| | WSS | |
| α chain w/alternative | MALVTSITVLLSLGIMGDAKTTQPNSMESNEEEPVHLPCNHSTISGTDYIHW | 1129 |
| signal peptide, Cα | YRQLPSQGPEYVIHGLTSNVNNRMASLAIAEDRKSSTLILHRATLRDAAVYY | |
| (substituted) | CILRDNNARLMFGDGTQLVVKPNIQNPEPAVYQLKDPRSQDSTLCLFTDFDS | |
| | QINVPKTMESGTFITDKCVLDMKAMDSKSNGAIAWSNQTSFTCQDIFKETNA | |
| | TYPSSDVPCDATLTEKSFETDMNLNFQNLLVIVLRILLLKVAGFNLLMTLRL | |
| | WSS | |
| α chain w/alternative | MHLVTSITVLLSLGIMGDAKTTQPNSMESNEEEPVHLPCNHSTISGTDYIHW | 1130 |
| signal peptide, Cα | YRQLPSQGPEYVIHGLTSNVNNRMASLAIAEDRKSSTLILHRATLRDAAVYY | |
| (substituted) | CILRDNNARLMFGDGTQLVVKPNIQNPEPAVYQLKDPRSQDSTLCLFTDFDS | |
| | QINVPKTMESGTFITDKCVLDMKAMDSKSNGAIAWSNQTSFTCQDIFKETNA | |
| | TYPSSDVPCDATLTEKSFETDMNLNFQNLLVIVLRILLLKVAGFNLLMTLRL | |
| | WSS | |
| CDR1β | MNHEY | 2121 |
| CDR2β | SMNVEV | 2122 |
| CDR3β | ASGLVGFNQPQH | 2123 |
| Vβ w/o signal peptide | QVTQNPRYLITVTGKKLTVTCSQNMNHEYMSWYRQDPGLGLRQIYYSMNVEV | 2124 |
| (SignalP) | TDKGDVPEGYKVSRKEKRNFPLILESPSPNQTSLYFCASGLVGFNQPQHFGD | |
| | GTRLSIL | |
| Vβ w/o signal peptide | EAQVTQNPRYLITVTGKKLTVTCSQNMNHEYMSWYRQDPGLGLRQIYYSMNV | 2125 |
| (IMGT) | EVTDKGDVPEGYKVSRKEKRNFPLILESPSPNQTSLYFCASGLVGFNQPQHF | |
| | GDGTRLSIL | |
| Vβ | MXPQLLGYVVLCLLGAGPLEAQVTQNPRYLITVTGKKLTVTCSQNMNHEYMS | 2126 |
| | WYRQDPGLGLRQIYYSMNVEVTDKGDVPEGYKVSRKEKRNFPLILESPSPNQ | 2127 |
| | TSLYFCASGLVGFNQPQHFGDGTRLSIL | |
| | (X = any amino acid) | |
| β chain w/WT signal | MGPQLLGYVVLCLLGAGPLEAQVTQNPRYLITVTGKKLTVTCSQNMNHEYMS | 2128 |
| peptide, Cβ | WYRQDPGLGLRQIYYSMNVEVTDKGDVPEGYKVSRKEKRNFPLILESPSPNQ | |
| (substituted) | TSLYFCASGLVGFNQPQHFGDGTRLSILEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MAPQLLGYVVLCLLGAGPLEAQVTQNPRYLITVTGKKLTVTCSQNMNHEYMS | 2129 |
| signal peptide, Cβ | WYRQDPGLGLRQIYYSMNVEVTDKGDVPEGYKVSRKEKRNFPLILESPSPNQ | |
| (substituted) | TSLYFCASGLVGFNQPQHFGDGTRLSILEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MHPQLLGYVVLCLLGAGPLEAQVTQNPRYLITVTGKKLTVTCSQNMNHEYMS | 2130 |
| signal peptide, Cβ | WYRQDPGLGLRQIYYSMNVEVTDKGDVPEGYKVSRKEKRNFPLILESPSPNQ | |
| (substituted) | TSLYFCASGLVGFNQPQHFGDGTRLSILEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR013 interacts with and/or is specific for p53. In some embodiments, the peptide is from a neoantigen of p53. In some embodiments, the neoantigen has the amino acid change R175H relative to the wild type p53 sequence. In some embodiments, TCR013 interacts with the neoantigen in the context of HLA-DRB1*13:01, as described in International Publication No. WO 2020/264269, incorporated herein by reference in its entirety.

TABLE 6N
Amino acid sequences of TCR014

| | | SEQ |
|---|---|---|
| | | ID |
| Description | Sequence | NO: |
| CDR1α | VSGNPY | 1131 |
| CDR2α | YITGDNLV | 1132 |
| CDR3α | AVRDGSATSGTYKYI | 1133 |
| Vα w/o signal peptide | QSVAQPEDQVNVAEGNPLTVKCTYSVSGNPYLFWYVQYPNRGLQFLLKYITG | 1134 |
| (SignalP) | DNLVKGSYGFEAEFNKSQTSFHLKKPSALVSDSALYFCAVRDGSATSGTYKY | |
| | IFGTGTRLKVLA | |
| Vα w/o signal peptide | AQSVAQPEDQVNVAEGNPLTVKCTYSVSGNPYLFWYVQYPNRGLQFLLKYIT | 1135 |
| (IMGT) | GDNLVKGSYGFEAEFNKSQTSFHLKKPSALVSDSALYFCAVRDGSATSGTYK | |
| | YIFGTGTRLKVLA | |
| Vα | MXSAPISMLAMLFTLSGLRAQSVAQPEDQVNVAEGNPLTVKCTYSVSGNPYL | 1136 |
| | FWYVQYPNRGLQFLLKYITGDNLVKGSYGFEAEFNKSQTSFHLKKPSALVSD | 1137 |
| | SALYFCAVRDGSATSGTYKYIFGTGTRLKVLA | |
| | (X = any amino acid) | |
| α chain w/WT signal | MASAPISMLAMLFTLSGLRAQSVAQPEDQVNVAEGNPLTVKCTYSVSGNPYL | 1138 |
| peptide, Cα | FWYVQYPNRGLQFLLKYITGDNLVKGSYGFEAEFNKSQTSFHLKKPSALVSD | 1139 |
| (substituted) | SALYFCAVRDGSATSGTYKYIFGTGTRLKVLANIQNPEPAVYQLKDPRSQDS | |
| | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| α chain w/alternative | MHSAPISMLAMLFTLSGLRAQSVAQPEDQVNVAEGNPLTVKCTYSVSGNPYL | 1140 |
| signal peptide, Cα | FWYVQYPNRGLQFLLKYITGDNLVKGSYGFEAEFNKSQTSFHLKKPSALVSD | |
| (substituted) | SALYFCAVRDGSATSGTYKYIFGTGTRLKVLANIQNPEPAVYQLKDPRSQDS | |
| | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| CDR1β | SEHNR | 2131 |
| CDR2β | FQNEAQ | 2132 |
| CDR3β | ASSPGLAYEQY | 2133 |
| Vβ w/o signal peptide | DTGVSQDPRHKITKRGQNVTFRCDPISEHNRLYWYRQTLGQGPEFLTYFQNE | 2134 |
| (SignalP) | AQLEKSRLLSDRFSAERPKGSFSTLEIQRTEQGDSAMYLCASSPGLAYEQYF | |
| | GPGTRLTVT | |
| Vβ w/o signal peptide | DTGVSQDPRHKITKRGQNVTFRCDPISEHNRLYWYRQTLGQGPEFLTYFQNE | 2135 |
| (IMGT) | AQLEKSRLLSDRFSAERPKGSFSTLEIQRTEQGDSAMYLCASSPGLAYEQYF | |
| | GPGTRLTVT | |
| Vβ | MXTSLLCWMALCLLGADHADTGVSQDPRHKITKRGQNVTFRCDPISEHNRLY | 2136 |
| | WYRQTLGQGPEFLTYFQNEAQLEKSRLLSDRESAERPKGSFSTLEIQRTEQG | 2137 |
| | DSAMYLCASSPGLAYEQYFGPGTRLTVT | |
| | (X = any amino acid) | |
| β chain w/WT signal | MGTSLLCWMALCLLGADHADTGVSQDPRHKITKRGQNVTFRCDPISEHNRLY | 2138 |
| peptide, Cβ | WYRQTLGQGPEFLTYFQNEAQLEKSRLLSDRFSAERPKGSFSTLEIQRTEQG | |
| (substituted) | DSAMYLCASSPGLAYEQYFGPGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MATSLLCWMALCLLGADHADTGVSQDPRHKITKRGQNVTFRCDPISEHNRLY | 2139 |
| signal peptide, Cβ | WYRQTLGQGPEFLTYFQNEAQLEKSRLLSDRFSAERPKGSFSTLEIQRTEQG | |
| (substituted) | DSAMYLCASSPGLAYEQYFGPGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MHTSLLCWMALCLLGADHADTGVSQDPRHKITKRGQNVTFRCDPISEHNRLY | 2140 |
| signal peptide, Cβ | WYRQTLGQGPEFLTYFQNEAQLEKSRLLSDRFSAERPKGSFSTLEIQRTEQG | |
| (substituted) | DSAMYLCASSPGLAYEQYFGPGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR014 interacts with and/or is specific for p53. In some embodiments, the peptide is from a neoantigen of p53. In some embodiments, the neoantigen has the amino acid change Y220C relative to the wild type p53 sequence. In some embodiments, TCR014 interacts with the neoantigen in the context of HLA-A*02:01, as described in International Publication No. WO 2020/264269, incorporated herein by reference in its entirety.

TABLE 6O
Amino acid sequences of TCR015.

| | | SEQ |
|---|---|---|
| | | ID |
| Description | Sequence | NO: |
| CDR1α | DRGSQS | 1141 |
| CDR2α | IYSNGD | 1142 |
| CDR3α | AWNSGGSNYKLT | 1143 |
| Vα w/o signal peptide | QQKEVEQNSGPLSVPEGAIASLNCTYSDRGSQSFFWYRQYSGKSPELIMFIY | 1144 |
| (SignalP) | SNGDKEDGRFTAQLNKASQYVSLLIRDSQPSDSATYLCAWNSGGSNYKLTFG | |
| | KGTLLTVNP | |
| Vα w/o signal peptide | QKEVEQNSGPLSVPEGAIASLNCTYSDRGSQSFFWYRQYSGKSPELIMFIYS | 1145 |
| (IMGT) | NGDKEDGRFTAQLNKASQYVSLLIRDSQPSDSATYLCAWNSGGSNYKLTFGK | |
| | GTLLTVNP | |
| Vα | MXSLRVLLVILWLQLSWVWSQQKEVEQNSGPLSVPEGAIASLNCTYSDRGSQ | 1146 |
| | SFFWYRQYSGKSPELIMFIYSNGDKEDGRFTAQLNKASQYVSLLIRDSQPSD | 1147 |
| | SATYLCAWNSGGSNYKLTFGKGTLLTVNP | |
| | (X = any amino acid) | |
| α chain w/WT signal | MKSLRVLLVILWLQLSWVWSQQKEVEQNSGPLSVPEGAIASLNCTYSDRGSQ | 1148 |
| peptide, Cα | SFFWYRQYSGKSPELIMFIYSNGDKEDGRFTAQLNKASQYVSLLIRDSQPSD | |
| (substituted) | SATYLCAWNSGGSNYKLTFGKGTLLTVNPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain w/alternative | MASLRVLLVILWLQLSWVWSQQKEVEQNSGPLSVPEGAIASLNCTYSDRGSQ | 1149 |
| signal peptide, Cα | SFFWYRQYSGKSPELIMFIYSNGDKEDGRFTAQLNKASQYVSLLIRDSQPSD | |
| (substituted) | SATYLCAWNSGGSNYKLTFGKGTLLTVNPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain w/alternative | MHSLRVLLVILWLQLSWVWSQQKEVEQNSGPLSVPEGAIASLNCTYSDRGSQ | 1150 |
| signal peptide, Cα | SFFWYRQYSGKSPELIMFIYSNGDKEDGRFTAQLNKASQYVSLLIRDSQPSD | |
| (substituted) | SATYLCAWNSGGSNYKLTFGKGTLLTVNPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| CDR1β | MNHEY | 2141 |
| CDR2β | SVGEGT | 2142 |
| CDR3β | ASSYSQAWGQPQH | 2143 |
| Vβ w/o signal peptide | GVTQTPKFRVLKTGQSMTLLCAQDMNHEYMYWYRQDPGMGLRLIHYSVGEGT | 2144 |
| (SignalP) | TAKGEVPDGYNVSRLKKQNFLLGLESAAPSQTSVYFCASSYSQAWGQPQHFG | |
| | DGTRLSIL | |
| Vβ w/o signal peptide | NAGVTQTPKFRVLKTGQSMTLLCAQDMNHEYMYWYRQDPGMGLRLIHYSVGE | 2145 |
| (IMGT) | GTTAKGEVPDGYNVSRLKKQNFLLGLESAAPSQTSVYFCASSYSQAWGQPQH | |
| | FGDGTRLSIL | |
| Vβ | MXLGLLCCGAFSLLWAGPVNAGVTQTPKFRVLKTGQSMTLLCAQDMNHEYMY | 2146 |
| | WYRQDPGMGLRLIHYSVGEGTTAKGEVPDGYNVSRLKKQNFLLGLESAAPSQ | 2147 |
| | TSVYFCASSYSQAWGQPQHFGDGIRLSIL | |
| | (X = any amino acid) | |
| β chain w/WT signal | MSLGLLCCGAFSLLWAGPVNAGVTQTPKFRVLKTGQSMTLLCAQDMNHEYMY | 2148 |
| peptide, Cβ | WYRQDPGMGLRLIHYSVGEGTTAKGEVPDGYNVSRLKKQNFLLGLESAAPSQ | |
| (substituted) | TSVYFCASSYSQAWGQPQHFGDGTRLSILEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/alternative | MALGLLCCGAFSLLWAGPVNAGVTQTPKFRVLKTGQSMTLLCAQDMNHEYMY | 2149 |
| signal peptide, Cβ | WYRQDPGMGLRLIHYSVGEGTTAKGEVPDGYNVSRLKKQNFLLGLESAAPSQ | |
| (substituted) | TSVYFCASSYSQAWGQPQHFGDGTRLSILEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/alternative | MHLGLLCCGAFSLLWAGPVNAGVTQTPKFRVLKTGQSMTLLCAQDMNHEYMY | 2150 |
| signal peptide, Cβ | WYRQDPGMGLRLIHYSVGEGTTAKGEVPDGYNVSRLKKQNFLLGLESAAPSQ | |
| (substituted) | TSVYFCASSYSQAWGQPQHFGDGTRLSILEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR015 interacts with and/or is specific for p53. In some embodiments, the peptide is from a neoantigen of p53. In some embodiments, the neoantigen has the amino acid change Y220C relative to the wild type p53 sequence. In some embodiments, TCR015 interacts with the neoantigen in the context of HLA-DRB1*04:01:01, as described in International Publication No. WO 2019/067243, incorporated herein by reference in its entirety.

TABLE 6P
Amino acid sequences of TCR016.

| | | SEQ |
|---|---|---|
| | | ID |
| Description | Sequence | NO: |
| CDR1α | VSGNPY | 1151 |
| CDR2α | YITGDNLV | 1152 |
| CDR3α | AVRVWDYKLS | 1153 |
| Vα w/o signal peptide | QSVAQPEDQVNVAEGNPLTVKCTYSVSGNPYLFWYVQYPNRGLQFLLKYITG | 1154 |
| (SignalP) | DNLVKGSYGFEAEFNKSQTSFHLKKPSALVSDSALYFCAVRVWDYKLSFGAG | |
| | TTVTVRA | |
| Vα w/o signal peptide | AQSVAQPEDQVNVAEGNPLTVKCTYSVSGNPYLFWYVQYPNRGLQFLLKYIT | 1155 |
| (IMGT) | GDNLVKGSYGFEAEFNKSQTSFHLKKPSALVSDSALYFCAVRVWDYKLSFGA | |
| | GTTVTVRA | |
| Vα | MXSAPISMLAMLFTLSGLRAQSVAQPEDQVNVAEGNPLTVKCTYSVSGNPYL | 1156 |
| | FWYVQYPNRGLQFLLKYITGDNLVKGSYGFEAEFNKSQTSFHLKKPSALVSD | 1157 |
| | SALYFCAVRVWDYKLSFGAGTTVTVRA | |
| | (X = any amino acid) | |
| α chain w/WT signal | MASAPISMLAMLFTLSGLRAQSVAQPEDQVNVAEGNPLTVKCTYSVSGNPYL | 1158 |
| peptide, Cα | FWYVQYPNRGLQFLLKYITGDNLVKGSYGFEAEFNKSQTSFHLKKPSALVSD | 1159 |
| (substituted) | SALYFCAVRVWDYKLSFGAGTTVTVRANIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain w/alternative | MHSAPISMLAMLFTLSGLRAQSVAQPEDQVNVAEGNPLTVKCTYSVSGNPYL | 1160 |
| signal peptide, Cα | FWYVQYPNRGLQFLLKYITGDNLVKGSYGFEAEFNKSQTSFHLKKPSALVSD | |
| (substituted) | SALYFCAVRVWDYKLSFGAGTTVTVRANIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| CDR1β | LNHDA | 2151 |
| CDR2β | SQIVND | 2152 |
| CDR3β | ASSISAGGDGYT | 2153 |
| Vβ w/o signal peptide | GITQSPKYLFRKEGQNVTLSCEQNLNHDAMYWYRQDPGQGLRLIYYSQIVND | 2154 |
| (SignalP) | FQKGDIAEGYSVSREKKESFPLTVTSAQKNPTAFYLCASSISAGGDGYTFGS | |
| | GTRLTVV | |
| Vβ w/o signal peptide | DGGITQSPKYLFRKEGQNVTLSCEQNLNHDAMYWYRQDPGQGLRLIYYSQIV | 2155 |
| (IMGT) | NDFQKGDIAEGYSVSREKKESFPLTVTSAQKNPTAFYLCASSISAGGDGYTF | |
| | GSGTRLTVV | |
| Vβ | MXNQVLCCVVLCFLGANTVDGGITQSPKYLFRKEGQNVTLSCEQNLNHDAMY | 2156 |
| | WYRQDPGQGLRLIYYSQIVNDFQKGDIAEGYSVSREKKESFPLTVTSAQKNP | 2157 |
| | TAFYLCASSISAGGDGYTFGSGTRLTVV | |
| | (X = any amino acid) | |
| β chain w/WT signal | MSNQVLCCVVLCFLGANTVDGGITQSPKYLFRKEGQNVTLSCEQNLNHDAMY | 2158 |
| peptide, Cβ | WYRQDPGQGLRLIYYSQIVNDFQKGDIAEGYSVSREKKESFPLTVTSAQKNP | |
| (substituted) | TAFYLCASSISAGGDGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MANQVLCCVVLCFLGANTVDGGITQSPKYLFRKEGQNVTLSCEQNLNHDAMY | 2159 |
| signal peptide, Cβ | WYRQDPGQGLRLIYYSQIVNDFQKGDIAEGYSVSREKKESFPLTVTSAQKNP | |
| (substituted) | TAFYLCASSISAGGDGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MHNQVLCCVVLCFLGANTVDGGITQSPKYLFRKEGQNVTLSCEQNLNHDAMY | 2160 |
| signal peptide, Cβ | WYRQDPGQGLRLIYYSQIVNDFQKGDIAEGYSVSREKKESFPLTVTSAQKNP | |
| (substituted) | TAFYLCASSISAGGDGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR016 interacts with and/or is specific for p53. In some embodiments, the peptide is from a neoantigen of p53. In some embodiments, the neoantigen has the amino acid change Y220C relative to the wild type p53 sequence. In some embodiments, TCR016 interacts with the neoantigen in the context of HLA-DRB3*02:02, as described in International Publication No. WO 2019/067243, incorporated herein by reference in its entirety.

TABLE 6Q
Amino acid sequences of TCR017.

| | | SEQ |
|---|---|---|
| | | ID |
| Description | Sequence | NO: |
| CDR1α | TSGFNG | 1161 |
| CDR2α | NVLDGL | 1162 |
| CDR3α | AVKWTGGFKTI | 1163 |
| Vα w/o signal peptide | QNIDQPTEMTATEGAIVQINCTYQTSGFNGLFWYQQHAGEAPTFLSYNVLDG | 1164 |
| (SignalP) | LEEKGRFSSFLSRSKGYSYLLLKELQMKDSASYLCAVKWTGGFKTIFGAGTR | |
| | LFVKA | |
| Vα w/o signal peptide | GQNIDQPTEMTATEGAIVQINCTYQTSGFNGLFWYQQHAGEAPTFLSYNVLD | 1165 |
| (IMGT) | GLEEKGRFSSFLSRSKGYSYLLLKELQMKDSASYLCAVKWTGGFKTIFGAGT | |
| | RLFVKA | |
| Vα | MXGVFLLYVSMKMGGTTGQNIDQPTEMTATEGAIVQINCTYQTSGFNGLFWY | 1166 |
| | QQHAGEAPTFLSYNVLDGLEEKGRESSFLSRSKGYSYLLLKELQMKDSASYL | 1167 |
| | CAVKWTGGFKTIFGAGTRLFVKA | |
| | (X = any amino acid) | |
| α chain w/WT signal | MWGVFLLYVSMKMGGTTGQNIDQPTEMTATEGAIVQINCTYQTSGFNGLFWY | 1168 |
| peptide, Cα | QQHAGEAPTFLSYNVLDGLEEKGRESSFLSRSKGYSYLLLKELQMKDSASYL | |
| (substituted) | CAVKWTGGFKTIFGAGTRLFVKANIQNPEPAVYQLKDPRSQDSTLCLFTDFD | |
| | SQINVPKTMESGTFITDKCVLDMKAMDSKSNGAIAWSNQTSFTCQDIFKETN | |
| | ATYPSSDVPCDATLTEKSFETDMNLNFQNLLVIVLRILLLKVAGFNLLMTLR | |
| | LWSS | |
| α chain w/alternative | MAGVFLLYVSMKMGGTTGQNIDQPTEMTATEGAIVQINCTYQTSGFNGLFWY | 1169 |
| signal peptide, Cα | QQHAGEAPTFLSYNVLDGLEEKGRESSFLSRSKGYSYLLLKELQMKDSASYL | |
| (substituted) | CAVKWTGGFKTIFGAGTRLFVKANIQNPEPAVYQLKDPRSQDSTLCLFTDFD | |
| | SQINVPKTMESGTFITDKCVLDMKAMDSKSNGAIAWSNQTSFTCQDIFKETN | |
| | ATYPSSDVPCDATLTEKSFETDMNLNFQNLLVIVLRILLLKVAGFNLLMTLR | |
| | LWSS | |
| α chain w/alternative | MHGVFLLYVSMKMGGTTGQNIDQPTEMTATEGAIVQINCTYQTSGFNGLFWY | 1170 |
| signal peptide, Cα | QQHAGEAPTFLSYNVLDGLEEKGRESSFLSRSKGYSYLLLKELQMKDSASYL | |
| (substituted) | CAVKWTGGFKTIFGAGTRLFVKANIQNPEPAVYQLKDPRSQDSTLCLFTDFD | |
| | SQINVPKTMESGTFITDKCVLDMKAMDSKSNGAIAWSNQTSFTCQDIFKETN | |
| | ATYPSSDVPCDATLTEKSFETDMNLNFQNLLVIVLRILLLKVAGFNLLMTLR | |
| | LWSS | |
| CDR1β | MNHEY | 2161 |
| CDR2β | SVGAGI | 2162 |
| CDR3β | ASSYRESHYGYT | 2163 |
| Vβ w/o signal peptide | GVTQTPKFQVLKTGQSMTLQCAQDMNHEYMSWYRQDPGMGLRLIHYSVGAGI | 2164 |
| (SignalP) | TDQGEVPNGYNVSRSTTEDFPLRLLSAAPSQTSVYFCASSYRESHYGYTFGS | |
| | GTRLTVV | |
| Vβ w/o signal peptide | NAGVTQTPKFQVLKTGQSMTLQCAQDMNHEYMSWYRQDPGMGLRLIHYSVGA | 2165 |
| (IMGT) | GITDQGEVPNGYNVSRSTTEDFPLRLLSAAPSQTSVYFCASSYRESHYGYTF | |
| | GSGTRLTVV | |
| Vβ | MXIGLLCCAALSLLWAGPVNAGVTQTPKFQVLKTGQSMTLQCAQDMNHEYMS | 2166 |
| | WYRQDPGMGLRLIHYSVGAGITDQGEVPNGYNVSRSTTEDFPLRLLSAAPSQ | 2167 |
| | TSVYFCASSYRESHYGYTFGSGTRLTVV | |
| | (X = any amino acid) | |
| β chain w/WT signal | MSIGLLCCAALSLLWAGPVNAGVTQTPKFQVLKTGQSMTLQCAQDMNHEYMS | 2168 |
| peptide, Cβ | WYRQDPGMGLRLIHYSVGAGITDQGEVPNGYNVSRSTTEDFPLRLLSAAPSQ | |
| (substituted) | TSVYFCASSYRESHYGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MAIGLLCCAALSLLWAGPVNAGVTQTPKFQVLKTGQSMTLQCAQDMNHEYMS | 2169 |
| signal peptide, Cβ | WYRQDPGMGLRLIHYSVGAGITDQGEVPNGYNVSRSTTEDFPLRLLSAAPSQ | |
| (substituted) | TSVYFCASSYRESHYGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MHIGLLCCAALSLLWAGPVNAGVTQTPKFQVLKTGQSMTLQCAQDMNHEYMS | 2170 |
| signal peptide, Cβ | WYRQDPGMGLRLIHYSVGAGITDQGEVPNGYNVSRSTTEDFPLRLLSAAPSQ | |
| (substituted) | TSVYFCASSYRESHYGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR017 interacts with and/or is specific for p53. In some embodiments, the peptide is from a neoantigen of p53. In some embodiments, the neoantigen has the amino acid change G245S relative to the wild type p53 sequence. In some embodiments, TCR017 interacts with the neoantigen in the context of HLA-DRB3*02:02, as described in International Publication No. WO 2019/067243, incorporated herein by reference in its entirety.

TABLE 6R
Amino acid sequences of TCR018.

| | | SEQ |
|---|---|---|
| | | ID |
| Description | Sequence | NO: |
| CDR1α | YGGTVN | 1171 |
| CDR2α | YFSGDPLV | 1172 |
| CDR3α | AVKGDYKLS | 1173 |
| Vα w/o signal peptide | QSVSQHNHHVILSEAASLELGCNYSYGGTVNLFWYVQYPGQHLQLLLKYFSG | 1174 |
| (SignalP) | DPLVKGIKGFEAEFIKSKFSFNLRKPSVQWSDTAEYFCAVKGDYKLSFGAGT | |
| | TVTVRA | |
| Vα w/o signal peptide | AQSVSQHNHHVILSEAASLELGCNYSYGGTVNLFWYVQYPGQHLQLLLKYFS | 1175 |
| (IMGT) | GDPLVKGIKGFEAEFIKSKFSFNLRKPSVQWSDTAEYFCAVKGDYKLSFGAG | |
| | TTVTVRA | |
| Vα | MXLLLIPVLGMIFALRDARAQSVSQHNHHVILSEAASLELGCNYSYGGTVNL | 1176 |
| | FWYVQYPGQHLQLLLKYFSGDPLVKGIKGFEAEFIKSKFSFNLRKPSVQWSD | 1177 |
| | TAEYFCAVKGDYKLSFGAGTTVTVRA | |
| | (X = any amino acid) | |
| α chain w/WT signal | MLLLLIPVLGMIFALRDARAQSVSQHNHHVILSEAASLELGCNYSYGGTVNL | 1178 |
| peptide, Cα | FWYVQYPGQHLQLLLKYFSGDPLVKGIKGFEAEFIKSKFSFNLRKPSVQWSD | |
| (substituted) | TAEYFCAVKGDYKLSFGAGTTVTVRANIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain w/alternative | MALLLIPVLGMIFALRDARAQSVSQHNHHVILSEAASLELGCNYSYGGTVNL | 1179 |
| signal peptide, Cα | FWYVQYPGQHLQLLLKYFSGDPLVKGIKGFEAEFIKSKFSFNLRKPSVQWSD | |
| (substituted) | TAEYFCAVKGDYKLSFGAGTTVTVRANIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain w/alternative | MHLLLIPVLGMIFALRDARAQSVSQHNHHVILSEAASLELGCNYSYGGTVNL | 1180 |
| signal peptide, Cα | FWYVQYPGQHLQLLLKYFSGDPLVKGIKGFEAEFIKSKFSFNLRKPSVQWSD | |
| (substituted) | TAEYFCAVKGDYKLSFGAGTTVTVRANIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| CDR1β | SGHAT | 2171 |
| CDR2β | FQNNGV | 2172 |
| CDR3β | ASSLVNTEAF | 2173 |
| Vβ w/o signal peptide | GVAQSPRYKIIEKRQSVAFWCNPISGHATLYWYQQILGQGPKLLIQFQNNGV | 2174 |
| (SignalP) | VDDSQLPKDRFSAERLKGVDSTLKIQPAKLEDSAVYLCASSLVNTEAFFGQG | |
| | TRLTVV | |
| Vβ w/o signal peptide | EAGVAQSPRYKIIEKRQSVAFWCNPISGHATLYWYQQILGQGPKLLIQFQNN | 2175 |
| (IMGT) | GVVDDSQLPKDRFSAERLKGVDSTLKIQPAKLEDSAVYLCASSLVNTEAFFG | |
| | QGTRLTVV | |
| Vβ | MXTRLLCWAALCLLGAELTEAGVAQSPRYKIIEKRQSVAFWCNPISGHATLY | 2176 |
| | WYQQILGQGPKLLIQFQNNGVVDDSQLPKDRFSAERLKGVDSTLKIQPAKLE | 2177 |
| | DSAVYLCASSLVNTEAFFGQGTRLTVV | |
| | (X = any amino acid) | |
| β chain w/WT signal | MGTRLLCWAALCLLGAELTEAGVAQSPRYKIIEKRQSVAFWCNPISGHATLY | 2178 |
| peptide, Cβ | WYQQILGQGPKLLIQFQNNGVVDDSQLPKDRFSAERLKGVDSTLKIQPAKLE | |
| (substituted) | DSAVYLCASSLVNTEAFFGQGTRLTVVEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain w/alternative | MATRLLCWAALCLLGAELTEAGVAQSPRYKIIEKRQSVAFWCNPISGHATLY | 2179 |
| signal peptide, Cβ | WYQQILGQGPKLLIQFQNNGVVDDSQLPKDRFSAERLKGVDSTLKIQPAKLE | |
| (substituted) | DSAVYLCASSLVNTEAFFGQGTRLTVVEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain w/alternative | MHTRLLCWAALCLLGAELTEAGVAQSPRYKIIEKRQSVAFWCNPISGHATLY | 2180 |
| signal peptide, Cβ | WYQQILGQGPKLLIQFQNNGVVDDSQLPKDRFSAERLKGVDSTLKIQPAKLE | |
| (substituted) | DSAVYLCASSLVNTEAFFGQGTRLTVVEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |

In some embodiments, TCR018 interacts with and/or is specific for p53. In some embodiments, the peptide is from a neoantigen of p53. In some embodiments, the neoantigen has the amino acid change G245S relative to the wild type p53 sequence. In some embodiments, TCR018 interacts with the neoantigen in the context of HLA-DRB3*02:02, as described in International Publication No. WO 2019/067243, incorporated herein by reference in its entirety.

TABLE 6S
Amino acid sequences of TCR019.

| | | SEQ |
|---|---|---|
| | | ID |
| Description | Sequence | NO: |
| CDR1α | TRDTTYY | 1181 |
| CDR2α | RNSFDEQN | 1182 |
| CDR3α | ALSEGGSNYKLT | 1183 |
| Vα w/o signal peptide | QKVTQAQTEISVVEKEDVTLDCVYETRDTTYYLFWYKQPPSGELVFLIRRNS | 1184 |
| (SignalP) | FDEQNEISGRYSWNFQKSTSSFNFTITASQVVDSAVYFCALSEGGSNYKLTF | |
| | GKGTLLTVNP | |
| Vα w/o signal peptide | AQKVTQAQTEISVVEKEDVTLDCVYETRDTTYYLFWYKQPPSGELVFLIRRN | 1185 |
| (IMGT) | SFDEQNEISGRYSWNFQKSTSSENFTITASQVVDSAVYFCALSEGGSNYKLT | |
| | FGKGTLLTVNP | |
| Vα | MXTASLLRAVIASICVVSSMAQKVTQAQTEISVVEKEDVTLDCVYETRDTTY | 1186 |
| | YLFWYKQPPSGELVFLIRRNSFDEQNEISGRYSWNFQKSTSSFNFTITASQV | 1187 |
| | VDSAVYFCALSEGGSNYKLTFGKGTLLTVNP | |
| | (X = any amino acid) | |
| α chain w/WT signal | MLTASLLRAVIASICVVSSMAQKVTQAQTEISVVEKEDVTLDCVYETRDTTY | 1188 |
| peptide, Cα | YLFWYKQPPSGELVFLIRRNSFDEQNEISGRYSWNFQKSTSSFNFTITASQV | |
| (substituted) | VDSAVYFCALSEGGSNYKLTFGKGTLLTVNPNIQNPEPAVYQLKDPRSQDST | |
| | LCLFTDFDSQINVPKTMESGTFITDKCVLDMKAMDSKSNGAIAWSNQTSFTC | |
| | QDIFKETNATYPSSDVPCDATLTEKSFETDMNLNFQNLLVIVLRILLLKVAG | |
| | FNLLMTLRLWSS | |
| α chain w/alternative | MATASLLRAVIASICVVSSMAQKVTQAQTEISVVEKEDVTLDCVYETRDTTY | 1189 |
| signal peptide, Cα | YLFWYKQPPSGELVFLIRRNSFDEQNEISGRYSWNFQKSTSSFNFTITASQV | |
| (substituted) | VDSAVYFCALSEGGSNYKLTFGKGTLLTVNPNIQNPEPAVYQLKDPRSQDST | |
| | LCLFTDFDSQINVPKTMESGTFITDKCVLDMKAMDSKSNGAIAWSNQTSFTC | |
| | QDIFKETNATYPSSDVPCDATLTEKSFETDMNLNFQNLLVIVLRILLLKVAG | |
| | FNLLMTLRLWSS | |
| α chain w/alternative | MHTASLLRAVIASICVVSSMAQKVTQAQTEISVVEKEDVTLDCVYETRDTTY | 1190 |
| signal peptide, Cα | YLFWYKQPPSGELVFLIRRNSFDEQNEISGRYSWNFQKSTSSFNFTITASQV | |
| (substituted) | VDSAVYFCALSEGGSNYKLTFGKGTLLTVNPNIQNPEPAVYQLKDPRSQDST | |
| | LCLFTDFDSQINVPKTMESGTFITDKCVLDMKAMDSKSNGAIAWSNQTSFTC | |
| | QDIFKETNATYPSSDVPCDATLTEKSFETDMNLNFQNLLVIVLRILLLKVAG | |
| | FNLLMTLRLWSS | |
| CDR1β | LNHNV | 2181 |
| CDR2β | YYDKDF | 2182 |
| CDR3β | ATSRELRGNEQF | 2183 |
| Vβ w/o signal peptide | DAMVIQNPRYQVTQFGKPVTLSCSQTLNHNVMYWYQQKSSQAPKLLFHYYDK | 2184 |
| (SignalP) | DFNNEADTPDNFQSRRPNTSFCFLDIRSPGLGDAAMYLCATSRELRGNEQFF | |
| | GPGTRLTVL | |
| Vβ w/o signal peptide | DAMVIQNPRYQVTQFGKPVTLSCSQTLNHNVMYWYQQKSSQAPKLLFHYYDK | 2185 |
| (IMGT) | DFNNEADTPDNFQSRRPNTSFCFLDIRSPGLGDAAMYLCATSRELRGNEQFF | |
| | GPGTRLTVL | |
| Vβ | MXPGLLHWMALCLLGTGHGDAMVIQNPRYQVTQFGKPVTLSCSQTLNHNVMY | 2186 |
| | WYQQKSSQAPKLLFHYYDKDFNNEADTPDNFQSRRPNTSFCFLDIRSPGLGD | 2187 |
| | AAMYLCATSRELRGNEQFFGPGTRLTVL | |
| | (X = any amino acid) | |
| β chain w/WT signal | MGPGLLHWMALCLLGTGHGDAMVIQNPRYQVTQFGKPVTLSCSQTLNHNVMY | 2188 |
| peptide, Cβ | WYQQKSSQAPKLLFHYYDKDFNNEADTPDNFQSRRPNTSFCFLDIRSPGLGD | |
| (substituted) | AAMYLCATSRELRGNEQFFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MAPGLLHWMALCLLGTGHGDAMVIQNPRYQVTQFGKPVTLSCSQTLNHNVMY | 2189 |
| signal peptide, Cβ | WYQQKSSQAPKLLFHYYDKDENNEADTPDNFQSRRPNTSFCFLDIRSPGLGD | |
| (substituted) | AAMYLCATSRELRGNEQFFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain w/alternative | MHPGLLHWMALCLLGTGHGDAMVIQNPRYQVTQFGKPVTLSCSQTLNHNVMY | 2190 |
| signal peptide, Cβ | WYQQKSSQAPKLLFHYYDKDFNNEADTPDNFQSRRPNTSFCFLDIRSPGLGD | |
| (substituted) | AAMYLCATSRELRGNEQFFGPGTRLTVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR019 interacts with and/or is specific for p53. In some embodiments, the peptide is from a neoantigen of p53. In some embodiments, the neoantigen has the amino acid change G245S relative to the wild type p53 sequence. In some embodiments, TCR019 interacts with the neoantigen in the context of HLA-DRB3*02:02, as described in International Publication No. WO 2019/067243, incorporated herein b reference in its entirety.

TABLE 6T
Amino acid sequences of TCR020.

| | | SEQ |
|---|---|---|
| | | ID |
| Description | Sequence | NO: |
| CDR1α | DRGSQS | 1191 |
| CDR2α | IYSNGD | 1192 |
| CDR3α | AVNDAGNMLT | 1193 |
| Vα without signal | QQKEVEQNSGPLSVPEGAIASLNCTYSDRGSQSFFWYRQYSGKSPELIMFIY | 1194 |
| peptide (SignalP) | SNGDKEDGRFTAQLNKASQYVSLLIRDSQPSDSATYLCAVNDAGNMLTFGGG | |
| | TRLMVKP | |
| Vα without signal | QKEVEQNSGPLSVPEGAIASLNCTYSDRGSQSFFWYRQYSGKSPELIMFIYS | 1195 |
| peptide (IMGT) | NGDKEDGRFTAQLNKASQYVSLLIRDSQPSDSATYLCAVNDAGNMLTFGGGT | |
| | RLMVKP | |
| Vα | MXSLRVLLVILWLQLSWVWSQQKEVEQNSGPLSVPEGAIASLNCTYSDRGSQ | 1196 |
| | SFFWYRQYSGKSPELIMFIYSNGDKEDGRFTAQLNKASQYVSLLIRDSQPSD | 1197 |
| | SATYLCAVNDAGNMLTFGGGTRLMVKP | |
| | (X = any amino acid) | |
| α chain with WT signal | MKSLRVLLVILWLQLSWVWSQQKEVEQNSGPLSVPEGAIASLNCTYSDRGSQ | 1198 |
| peptide, Cα | SFFWYRQYSGKSPELIMFIYSNGDKEDGRFTAQLNKASQYVSLLIRDSQPSD | |
| (substituted) | SATYLCAVNDAGNMLTFGGGTRLMVKPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain with | MASLRVLLVILWLQLSWVWSQQKEVEQNSGPLSVPEGAIASLNCTYSDRGSQ | 1199 |
| alternative signal | SFFWYRQYSGKSPELIMFIYSNGDKEDGRFTAQLNKASQYVSLLIRDSQPSD | |
| peptide, Cα | SATYLCAVNDAGNMLTFGGGTRLMVKPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain with | MHSLRVLLVILWLQLSWVWSQQKEVEQNSGPLSVPEGAIASLNCTYSDRGSQ | 1200 |
| alternative signal | SFFWYRQYSGKSPELIMFIYSNGDKEDGRFTAQLNKASQYVSLLIRDSQPSD | |
| peptide, Cα | SATYLCAVNDAGNMLTFGGGTRLMVKPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| CDR1β | DFQATT | 2191 |
| CDR2β | SNEGSKA | 2192 |
| CDR3β | SAAGQANTEAF | 2193 |
| Vβ without signal | GSGLGAVVSQHPSWVICKSGTSVKIECRSLDFQATTMFWYRQFPKQSLMLMA | 2194 |
| peptide (SignalP) | TSNEGSKATYEQGVEKDKFLINHASLTLSTLTVTSAHPEDSSFYICSAAGQA | |
| | NTEAFFGQGTRLTVV | |
| Vβ without signal | GAVVSQHPSWVICKSGTSVKIECRSLDFQATTMFWYRQFPKQSLMLMATSNE | 2195 |
| peptide (IMGT) | GSKATYEQGVEKDKFLINHASLTLSTLTVTSAHPEDSSFYICSAAGQANTEA | |
| | FFGQGTRLTVV | |
| Vβ | MXLLLLLLGPGISLLLPGSLAGSGLGAVVSQHPSWVICKSGTSVKIECRSLD | 2196 |
| | FQATTMFWYRQFPKQSLMLMATSNEGSKATYEQGVEKDKFLINHASLTLSTL | 2197 |
| | TVTSAHPEDSSFYICSAAGQANTEAFFGQGTRLTVV | |
| | (X = any amino acid) | |
| β chain with WT signal | MLLLLLLLGPGISLLLPGSLAGSGLGAVVSQHPSWVICKSGTSVKIECRSLD | 2198 |
| peptide, Cβ | FQATTMFWYRQFPKQSLMLMATSNEGSKATYEQGVEKDKFLINHASLTLSTL | |
| (substituted) | TVTSAHPEDSSFYICSAAGQANTEAFFGQGTRLTVVEDLRNVTPPKVSLFEP | |
| | SKAEIANKQKATLVCLARGFFPDHVELSWWVNGKEVHSGVCTDPQAYKESNY | |
| | SYCLSSRLRVSATFWHNPRNHFRCQVQFHGLSEEDKWPEGSPKPVTQNISAE | |
| | AWGRADCGITSASYQQGVLSATILYEILLGKATLYAVLVSTLVVMAMVKRKN | |
| | S | |
| β chain with alternative | MALLLLLLGPGISLLLPGSLAGSGLGAVVSQHPSWVICKSGTSVKIECRSLD | 2199 |
| signal peptide, Cβ | FQATTMFWYRQFPKQSLMLMATSNEGSKATYEQGVEKDKFLINHASLTLSTL | |
| (substituted) | TVTSAHPEDSSFYICSAAGQANTEAFFGQGTRLTVVEDLRNVTPPKVSLFEP | |
| | SKAEIANKQKATLVCLARGFFPDHVELSWWVNGKEVHSGVCTDPQAYKESNY | |
| | SYCLSSRLRVSATFWHNPRNHFRCQVQFHGLSEEDKWPEGSPKPVTQNISAE | |
| | AWGRADCGITSASYQQGVLSATILYEILLGKATLYAVLVSTLVVMAMVKRKN | |
| | S | |
| β chain with alternative | MHLLLLLLGPGISLLLPGSLAGSGLGAVVSQHPSWVICKSGTSVKIECRSLD | 2200 |
| signal peptide, Cβ | FQATTMFWYRQFPKQSLMLMATSNEGSKATYEQGVEKDKFLINHASLTLSTL | |
| (substituted) | TVTSAHPEDSSFYICSAAGQANTEAFFGQGTRLTVVEDLRNVTPPKVSLFEP | |
| | SKAEIANKQKATLVCLARGFFPDHVELSWWVNGKEVHSGVCTDPQAYKESNY | |
| | SYCLSSRLRVSATFWHNPRNHERCQVQFHGLSEEDKWPEGSPKPVTQNISAE | |
| | AWGRADCGITSASYQQGVLSATILYEILLGKATLYAVLVSTLVVMAMVKRKN | |
| | S | |

In some embodiments, TCR020 interacts with and/or is specific for p53. In some embodiments, the peptide is from a neoantigen of p53. In some embodiments, the neoantigen has the amino acid change G245S relative to the wild type p53 sequence. In some embodiments, TCR020 interacts with the neoantigen in the context of HLA-DRB3*02:02, as described in International Publication No. WO 2019/067243, incorporated herein by reference in its entirety.

TABLE 6U
Amino acid sequences of TCR021.

| | | SEQ |
|---|---|---|
| | | ID |
| Description | Sequence | NO: |
| CDR1α | TRDTTYY | 1201 |
| CDR2α | RNSFDEQN | 1202 |
| CDR3α | ALSEVDSGNTPLV | 1203 |
| Vα without signal | QKVTQAQTEISVVEKEDVTLDCVYETRDTTYYLFWYKQPPSGELVFLIRRNS | 1204 |
| peptide (SignalP) | FDEQNEISGRYSWNFQKSTSSFNFTITASQVVDSAVYFCALSEVDSGNTPLV | |
| | FGKGTRLSVIA | |
| Vα without signal | AQKVTQAQTEISVVEKEDVTLDCVYETRDTTYYLFWYKQPPSGELVFLIRRN | 1205 |
| peptide (IMGT) | SFDEQNEISGRYSWNFQKSTSSFNFTITASQVVDSAVYFCALSEVDSGNTPL | |
| | VFGKGTRLSVIA | |
| Vα | MXTASLLRAVIASICVVSSMAQKVTQAQTEISVVEKEDVTLDCVYETRDTTY | 1206 |
| | YLFWYKQPPSGELVFLIRRNSFDEQNEISGRYSWNFQKSTSSENFTITASQV | 1207 |
| | VDSAVYFCALSEVDSGNTPLVFGKGTRLSVIA | |
| | (X = any amino acid) | |
| α chain with WT signal | MLTASLLRAVIASICVVSSMAQKVTQAQTEISVVEKEDVTLDCVYETRDTTY | 1208 |
| peptide, Cα | YLFWYKQPPSGELVFLIRRNSFDEQNEISGRYSWNFQKSTSSFNFTITASQV | |
| (substituted) | VDSAVYFCALSEVDSGNTPLVFGKGTRLSVIANIQNPEPAVYQLKDPRSQDS | |
| | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| α chain with | MATASLLRAVIASICVVSSMAQKVTQAQTEISVVEKEDVTLDCVYETRDTTY | 1209 |
| alternative signal | YLFWYKQPPSGELVFLIRRNSFDEQNEISGRYSWNFQKSTSSFNFTITASQV | |
| peptide, Cα | VDSAVYFCALSEVDSGNTPLVFGKGTRLSVIANIQNPEPAVYQLKDPRSQDS | |
| (substituted) | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| α chain with | MHTASLLRAVIASICVVSSMAQKVTQAQTEISVVEKEDVTLDCVYETRDTTY | 1210 |
| alternative signal | YLFWYKQPPSGELVFLIRRNSFDEQNEISGRYSWNFQKSTSSFNFTITASQV | |
| peptide, Cα | VDSAVYFCALSEVDSGNTPLVFGKGTRLSVIANIQNPEPAVYQLKDPRSQDS | |
| (substituted) | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| CDR1β | SGDLS | 2201 |
| CDR2β | YYNGEE | 2202 |
| CDR3β | ASSVGSSSSTDTQY | 2203 |
| Vβ without signal | GVTQTPKHLITATGQRVTLRCSPRSGDLSVYWYQQSLDQGLQFLIQYYNGEE | 2204 |
| peptide (SignalP) | RAKGNILERFSAQQFPDLHSELNLSSLELGDSALYFCASSVGSSSSTDTQYF | |
| | GPGTRLTVL | |
| Vβ without signal | DSGVTQTPKHLITATGQRVTLRCSPRSGDLSVYWYQQSLDQGLQFLIQYYNG | 2205 |
| peptide (IMGT) | EERAKGNILERFSAQQFPDLHSELNLSSLELGDSALYFCASSVGSSSSTDTQ | |
| | YFGPGTRLTVL | |
| Vβ | MXHFRLLCCVAFCLLGAGPVDSGVTQTPKHLITATGQRVTLRCSPRSGDLSV | 2206 |
| | YWYQQSLDQGLQFLIQYYNGEERAKGNILERFSAQQFPDLHSELNLSSLELG | 2207 |
| | DSALYFCASSVGSSSSTDTQYFGPGTRLTVL | |
| | (X = any amino acid) | |
| β chain with WT signal | MGFRLLCCVAFCLLGAGPVDSGVTQTPKHLITATGQRVTLRCSPRSGDLSVY | 2208 |
| peptide, Cβ | WYQQSLDQGLQFLIQYYNGEERAKGNILERFSAQQFPDLHSELNLSSLELGD | |
| (substituted) | SALYFCASSVGSSSSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MAFRLLCCVAFCLLGAGPVDSGVTQTPKHLITATGQRVTLRCSPRSGDLSVY | 2209 |
| signal peptide, Cβ | WYQQSLDQGLQFLIQYYNGEERAKGNILERFSAQQFPDLHSELNLSSLELGD | |
| (substituted) | SALYFCASSVGSSSSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHFRLLCCVAFCLLGAGPVDSGVTQTPKHLITATGQRVTLRCSPRSGDLSVY | 2210 |
| signal peptide, Cβ | WYQQSLDQGLQFLIQYYNGEERAKGNILERFSAQQFPDLHSELNLSSLELGD | |
| (substituted) | SALYFCASSVGSSSSTDTQYFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR021 interacts with and/or is specific for p53. In some embodiments, the peptide is from a neoantigen of p53. In some embodiments, the neoantigen has the amino acid change R248Q relative to the wild type p53 sequence. In some embodiments, TCR021 interacts with the neoantigen in the context of HLA-A*02:01, as described in International Publication No. WO 2019/067243, incorporated herein by reference in its entirety.

TABLE 6V
Amino acid sequences of TCR022.

| | | SEQ |
|---|---|---|
| | | ID |
| Description | Sequence | NO: |
| CDR1α | NTAFDY | 1771 |
| CDR2α | IRPDVSE | 1772 |
| CDR3α | AAEAGNHRGSTLGRLY | 1773 |
| Vα w/o signal peptide | QQKEKSDQQQVKQSPQSLIVQKGGISIINCAYENTAFDYFPWYQQFPGKGPA | 1774 |
| (SignalP) | LLIAIRPDVSEKKEGRFTISENKSAKQFSLHIMDSQPGDSATYFCAAEAGNH | |
| | RGSTLGRLYFGRGTQLTVWP | |
| Vα w/o signal peptide | QQQVKQSPQSLIVQKGGISIINCAYENTAFDYFPWYQQFPGKGPALLIAIRP | 1775 |
| (IMGT) | DVSEKKEGRFTISFNKSAKQFSLHIMDSQPGDSATYFCAAEAGNHRGSTLGR | |
| | LYFGRGTQLTVWP | |
| Vα | MXKILGASFLVLWLQLCWVSGQQKEKSDQQQVKQSPQSLIVQKGGISIINCA | 1776 |
| | YENTAFDYFPWYQQFPGKGPALLIAIRPDVSEKKEGRFTISFNKSAKQFSLH | 1777 |
| | IMDSQPGDSATYFCAAEAGNHRGSTLGRLYFGRGTQLTVWP | |
| | (X = any amino acid) | |
| α chain with WT signal | MDKILGASFLVLWLQLCWVSGQQKEKSDQQQVKQSPQSLIVQKGGISIINCA | 1778 |
| peptide, Cα | YENTAFDYFPWYQQFPGKGPALLIAIRPDVSEKKEGRFTISFNKSAKQFSLH | |
| (substituted) | IMDSQPGDSATYFCAAEAGNHRGSTLGRLYFGRGTQLTVWPNIQNPEPAVYQ | |
| | LKDPRSQDSTLCLFTDFDSQINVPKTMESGTFITDKCVLDMKAMDSKSNGAI | |
| | AWSNQTSFTCQDIFKETNATYPSSDVPCDATLTEKSFETDMNLNFQNLLVIV | |
| | LRILLLKVAGFNLLMTLRLWSS | |
| α chain with | MAKILGASFLVLWLQLCWVSGQQKEKSDQQQVKQSPQSLIVQKGGISIINCA | 1779 |
| alternative signal | YENTAFDYFPWYQQFPGKGPALLIAIRPDVSEKKEGRFTISFNKSAKQFSLH | |
| peptide, Co | IMDSQPGDSATYFCAAEAGNHRGSTLGRLYFGRGTQLTVWPNIQNPEPAVYQ | |
| (substituted) | LKDPRSQDSTLCLFTDFDSQINVPKTMESGTFITDKCVLDMKAMDSKSNGAI | |
| | AWSNQTSFTCQDIFKETNATYPSSDVPCDATLTEKSFETDMNLNFQNLLVIV | |
| | LRILLLKVAGFNLLMTLRLWSS | |
| α chain with | MHKILGASFLVLWLQLCWVSGQQKEKSDQQQVKQSPQSLIVQKGGISIINCA | 1780 |
| alternative signal | YENTAFDYFPWYQQFPGKGPALLIAIRPDVSEKKEGRFTISFNKSAKQFSLH | |
| peptide, Cα | IMDSQPGDSATYFCAAEAGNHRGSTLGRLYFGRGTQLTVWPNIQNPEPAVYQ | |
| (substituted) | LKDPRSQDSTLCLFTDFDSQINVPKTMESGTFITDKCVLDMKAMDSKSNGAI | |
| | AWSNQTSFTCQDIFKETNATYPSSDVPCDATLTEKSFETDMNLNFQNLLVIV | |
| | LRILLLKVAGFNLLMTLRLWSS | |
| CDR1β | SGHRS | 2771 |
| CDR2β | YFSETQ | 2772 |
| CDR3β | ASSLAAGGYFNEQF | 2773 |
| Vβ w/o signal peptide | GVTQTPRYLIKTRGQQVTLSCSPISGHRSVSWYQQTPGQGLQFLFEYFSETQ | 2774 |
| (SignalP) | RNKGNFPGRFSGRQFSNSRSEMNVSTLELGDSALYLCASSLAAGGYFNEQFF | |
| | GPGTRLTVL | |
| Vβ w/o signal peptide | KAGVTQTPRYLIKTRGQQVTLSCSPISGHRSVSWYQQTPGQGLQFLFEYFSE | 2775 |
| (IMGT) | TQRNKGNFPGRFSGRQFSNSRSEMNVSTLELGDSALYLCASSLAAGGYFNEQ | |
| | FFGPGTRLTVL | |
| Vβ | MXSRLLCWVLLCLLGAGPVKAGVTQTPRYLIKTRGQQVTLSCSPISGHRSVS | 2776 |
| | WYQQTPGQGLQFLFEYFSETQRNKGNFPGRFSGRQFSNSRSEMNVSTLELGD | 2777 |
| | SALYLCASSLAAGGYFNEQFFGPGTRLTVL | |
| | (X = any amino acid) | |
| β chain with WT signal | MGSRLLCWVLLCLLGAGPVKAGVTQTPRYLIKTRGQQVTLSCSPISGHRSVS | 2778 |
| peptide, Cβ | WYQQTPGQGLQFLFEYFSETQRNKGNFPGRFSGRQFSNSRSEMNVSTLELGD | |
| (substituted) | SALYLCASSLAAGGYFNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MASRLLCWVLLCLLGAGPVKAGVTQTPRYLIKTRGQQVTLSCSPISGHRSVS | 2779 |
| signal peptide, Cβ | WYQQTPGQGLQFLFEYFSETQRNKGNFPGRFSGRQFSNSRSEMNVSTLELGD | |
| (substituted) | SALYLCASSLAAGGYFNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHSRLLCWVLLCLLGAGPVKAGVTQTPRYLIKTRGQQVTLSCSPISGHRSVS | 2780 |
| signal peptide, Cβ | WYQQTPGQGLQFLFEYFSETQRNKGNFPGRFSGRQFSNSRSEMNVSTLELGD | |
| (substituted) | SALYLCASSLAAGGYFNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR022 interacts with and/or is specific for KRAS. In some embodiments, the peptide is from a neoantigen of KRAS. In some embodiments, the neoantigen has the amino acid change G12D relative to the wild type KRAS sequence. In some embodiments, TCR022 interacts with the neoantigen in the context of HLA-A*11:01, as described in International Publication No. WO 2021/163434, incorporated herein by reference in its entirety.

TABLE 6W
Amino acid sequences of TCR023.

| | | SEQ |
|---|---|---|
| | | ID |
| Description | Sequence | NO: |
| CDR1α | NSAFQY | 1221 |
| CDR2α | TYSSGN | 1222 |
| CDR3α | AMTSPYNNNDMR | 1223 |
| Vα without signal | QQKEVEQDPGPLSVPEGAIVSLNCTYSNSAFQYFMWYRQYSRKGPELLMYTY | 1224 |
| peptide (SignalP) | SSGNKEDGRFTAQVDKSSKYISLFIRDSQPSDSATYLCAMTSPYNNNDMRFG | |
| | AGTRLTVKP | |
| Vα without signal | QKEVEQDPGPLSVPEGAIVSLNCTYSNSAFQYFMWYRQYSRKGPELLMYTYS | 1225 |
| peptide (IMGT) | SGNKEDGRFTAQVDKSSKYISLFIRDSQPSDSATYLCAMTSPYNNNDMRFGA | |
| | GTRLTVKP | |
| Vα | MXKSLRVLLVILWLQLSWVWSQQKEVEQDPGPLSVPEGAIVSLNCTYSNSAF | 1226 |
| | QYFMWYRQYSRKGPELLMYTYSSGNKEDGRFTAQVDKSSKYISLFIRDSQPS | 1227 |
| | DSATYLCAMTSPYNNNDMRFGAGTRLTVKP | |
| | (X = any amino acid) | |
| α chain with WT signal | MCKSLRVLLVILWLQLSWVWSQQKEVEQDPGPLSVPEGAIVSLNCTYSNSAF | 1228 |
| peptide, Cα | QYFMWYRQYSRKGPELLMYTYSSGNKEDGRFTAQVDKSSKYISLFIRDSQPS | |
| (substituted) | DSATYLCAMTSPYNNNDMRFGAGTRLTVKPNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MAKSLRVLLVILWLQLSWVWSQQKEVEQDPGPLSVPEGAIVSLNCTYSNSAF | 1229 |
| alternative signal | QYFMWYRQYSRKGPELLMYTYSSGNKEDGRFTAQVDKSSKYISLFIRDSQPS | |
| peptide, Cα | DSATYLCAMTSPYNNNDMRFGAGTRLTVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MHKSLRVLLVILWLQLSWVWSQQKEVEQDPGPLSVPEGAIVSLNCTYSNSAF | 1230 |
| alternative signal | QYFMWYRQYSRKGPELLMYTYSSGNKEDGRFTAQVDKSSKYISLFIRDSQPS | |
| peptide, Cα | DSATYLCAMTSPYNNNDMRFGAGTRLTVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| CDR1β | DFQATT | 2221 |
| CDR2β | SNEGSKA | 2222 |
| CDR3β | SGGLEEAARQFI | 2223 |
| Vβ without signal | GSGLGAVVSQHPSWVICKSGTSVKIECRSLDFQATTMFWYRQFPKQSLMLMA | 2224 |
| peptide (SignalP) | TSNEGSKATYEQGVEKDKFLINHASLTLSTLTVTSAHPEDSSFYICSGGLEE | |
| | AARQFIGPGTRLTVL | |
| Vβ without signal | GAVVSQHPSWVICKSGTSVKIECRSLDFQATTMFWYRQFPKQSLMLMATSNE | 2225 |
| peptide (IMGT) | GSKATYEQGVEKDKFLINHASLTLSTLTVTSAHPEDSSFYICSGGLEEAARQ | |
| | FIGPGTRLTVL | |
| Vβ | MXLLLLLLGPGISLLLPGSLAGSGLGAVVSQHPSWVICKSGTSVKIECRSLD | 2226 |
| | FQATTMFWYRQFPKQSLMLMATSNEGSKATYEQGVEKDKFLINHASLTLSTL | 2227 |
| | TVTSAHPEDSSFYICSGGLEEAARQFIGPGTRLTVL | |
| | (X = any amino acid) | |
| β chain with WT signal | MLLLLLLLGPGISLLLPGSLAGSGLGAVVSQHPSWVICKSGTSVKIECRSLD | 2228 |
| peptide, Cβ | FQATTMFWYRQFPKQSLMLMATSNEGSKATYEQGVEKDKFLINHASLTLSTL | |
| (substituted) | TVTSAHPEDSSFYICSGGLEEAARQFIGPGTRLTVLEDLRNVTPPKVSLFEP | |
| | SKAEIANKQKATLVCLARGFFPDHVELSWWVNGKEVHSGVCTDPQAYKESNY | |
| | SYCLSSRLRVSATFWHNPRNHFRCQVQFHGLSEEDKWPEGSPKPVTQNISAE | |
| | AWGRADCGITSASYQQGVLSATILYEILLGKATLYAVLVSTLVVMAMVKRKN | |
| | S | |
| β chain with alternative | MALLLLLLGPGISLLLPGSLAGSGLGAVVSQHPSWVICKSGTSVKIECRSLD | 2229 |
| signal peptide, Cβ | FQATTMFWYRQFPKQSLMLMATSNEGSKATYEQGVEKDKFLINHASLTLSTL | |
| (substituted) | TVTSAHPEDSSFYICSGGLEEAARQFIGPGTRLTVLEDLRNVTPPKVSLFEP | |
| | SKAEIANKQKATLVCLARGFFPDHVELSWWVNGKEVHSGVCTDPQAYKESNY | |
| | SYCLSSRLRVSATFWHNPRNHFRCQVQFHGLSEEDKWPEGSPKPVTQNISAE | |
| | AWGRADCGITSASYQQGVLSATILYEILLGKATLYAVLVSTLVVMAMVKRKN | |
| | S | |
| β chain with alternative | MHLLLLLLGPGISLLLPGSLAGSGLGAVVSQHPSWVICKSGTSVKIECRSLD | 2230 |
| signal peptide, Cβ | FQATTMFWYRQFPKQSLMLMATSNEGSKATYEQGVEKDKFLINHASLTLSTL | |
| (substituted) | TVTSAHPEDSSFYICSGGLEEAARQFIGPGTRLTVLEDLRNVTPPKVSLFEP | |
| | SKAEIANKQKATLVCLARGFFPDHVELSWWVNGKEVHSGVCTDPQAYKESNY | |
| | SYCLSSRLRVSATFWHNPRNHFRCQVQFHGLSEEDKWPEGSPKPVTQNISAE | |
| | AWGRADCGITSASYQQGVLSATILYEILLGKATLYAVLVSTLVVMAMVKRKN | |
| | S | |

In some embodiments, TCR023 interacts with and/or is specific for p53. In some embodiments, the peptide is from a neoantigen of p53. In some embodiments, the neoantigen has the amino acid change R248Q relative to the wild type p53 sequence. In some embodiments, TCR023 interacts with the neoantigen in the context of HLA-A*02:01, as described in International Publication No. WO 2019/067243, incorporated herein by reference in its entirety.

TABLE 6X
Amino acid sequences of TCR024.

| | | SEQ |
|---|---|---|
| | | ID |
| Description | Sequence | NO: |
| CDR1α | NSAFQY | 1231 |
| CDR2α | TYSSGN | 1232 |
| CDR3α | AMTSPYNNNDMR | 1233 |
| Vα without signal | QQKEVEQDPGPLSVPEGAIVSLNCTYSNSAFQYFMWYRQYSRKGPELLMYTY | 1234 |
| peptide (SignalP) | SSGNKEDGRFTAQVDKSSKYISLFIRDSQPSDSATYLCAMTSPYNNNDMRFG | |
| | AGTRLTVKP | |
| Vα without signal | QKEVEQDPGPLSVPEGAIVSLNCTYSNSAFQYFMWYRQYSRKGPELLMYTYS | 1235 |
| peptide (IMGT) | SGNKEDGRFTAQVDKSSKYISLFIRDSQPSDSATYLCAMTSPYNNNDMRFGA | |
| | GTRLTVKP | |
| Vα | MXKSLRVLLVILWLQLSWVWSQQKEVEQDPGPLSVPEGAIVSLNCTYSNSAF | 1236 |
| | QYFMWYRQYSRKGPELLMYTYSSGNKEDGRFTAQVDKSSKYISLFIRDSQPS | 1237 |
| | DSATYLCAMTSPYNNNDMRFGAGTRLTVKP | |
| | (X = any amino acid) | |
| α chain with WT signal | MCKSLRVLLVILWLQLSWVWSQQKEVEQDPGPLSVPEGAIVSLNCTYSNSAF | 1238 |
| peptide, Cα | QYFMWYRQYSRKGPELLMYTYSSGNKEDGRFTAQVDKSSKYISLFIRDSQPS | |
| (substituted) | DSATYLCAMTSPYNNNDMRFGAGTRLTVKPNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MAKSLRVLLVILWLQLSWVWSQQKEVEQDPGPLSVPEGAIVSLNCTYSNSAF | 1239 |
| alternative signal | QYFMWYRQYSRKGPELLMYTYSSGNKEDGRFTAQVDKSSKYISLFIRDSQPS | |
| peptide, Cα | DSATYLCAMTSPYNNNDMRFGAGTRLTVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MHKSLRVLLVILWLQLSWVWSQQKEVEQDPGPLSVPEGAIVSLNCTYSNSAF | 1240 |
| alternative signal | QYFMWYRQYSRKGPELLMYTYSSGNKEDGRFTAQVDKSSKYISLFIRDSQPS | |
| peptide, Cα | DSATYLCAMTSPYNNNDMRFGAGTRLTVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| CDR1β | DFQATT | 2231 |
| CDR2β | SNEGSKA | 2232 |
| CDR3β | SGGLEEAARQFI | 2233 |
| Vβ without signal | AVVSQHPSRVICKSGTSVKIECRSLDFQATTMFWYRQFPKQSLMLMATSNEG | 2234 |
| peptide (SignalP) | SKATYEQGVEKDKFLINHASLTLSTLTVTSAHPEDSSFYICSGGLEEAARQF | |
| | IGPGTRLTVL | |
| Vβ without signal | GAVVSQHPSRVICKSGTSVKIECRSLDFQATTMFWYRQFPKQSLMLMATSNE | 2235 |
| peptide (IMGT) | GSKATYEQGVEKDKFLINHASLTLSTLTVTSAHPEDSSFYICSGGLEEAARQ | |
| | FIGPGTRLTVL | |
| Vβ | MXLLLLLLGPAGSGLGAVVSQHPSRVICKSGTSVKIECRSLDFQATTMFWYR | 2236 |
| | QFPKQSLMLMATSNEGSKATYEQGVEKDKFLINHASLTLSTLTVTSAHPEDS | 2237 |
| | SFYICSGGLEEAARQFIGPGTRLTVL | |
| | (X = any amino acid) | |
| β chain with WT signal | MLLLLLLLGPAGSGLGAVVSQHPSRVICKSGTSVKIECRSLDFQATTMFWYR | 2238 |
| peptide, Cβ | QFPKQSLMLMATSNEGSKATYEQGVEKDKFLINHASLTLSTLTVTSAHPEDS | |
| (substituted) | SFYICSGGLEEAARQFIGPGTRLTVLEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain with alternative | MALLLLLLGPAGSGLGAVVSQHPSRVICKSGTSVKIECRSLDFQATTMFWYR | 2239 |
| signal peptide, Cβ | QFPKQSLMLMATSNEGSKATYEQGVEKDKFLINHASLTLSTLTVTSAHPEDS | |
| (substituted) | SFYICSGGLEEAARQFIGPGTRLTVLEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain with alternative | MHLLLLLLGPAGSGLGAVVSQHPSRVICKSGTSVKIECRSLDFQATTMFWYR | 2240 |
| signal peptide, Cβ | QFPKQSLMLMATSNEGSKATYEQGVEKDKFLINHASLTLSTLTVTSAHPEDS | |
| (substituted) | SFYICSGGLEEAARQFIGPGTRLTVLEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |

In some embodiments, TCR024 interacts with and/or is specific for p53. In some embodiments, the peptide is from a neoantigen of p53. In some embodiments, the neoantigen has the amino acid change R248Q relative to the wild type p53 sequence. In some embodiments, TCR024 interacts with the neoantigen in the context of HLA-A*02:01, as described in International Publication No. WO 2019/067243, incorporated herein by reference in its entirety.

TABLE 6Y
Amino acid sequences of TCR025.

| | | SEQ |
|---|---|---|
| | | ID |
| Description | Sequence | NO: |
| CDR1α | TISGNEY | 1241 |
| CDR2α | GLKNN | 1242 |
| CDR3α | IVPNDYKLS | 1243 |
| Vα without signal | KTTQPPSMDCAEGRAANLPCNHSTISGNEYVYWYRQIHSQGPQYIIHGLKNN | 1244 |
| peptide (SignalP) | ETNEMASLIITEDRKSSTLILPHATLRDTAVYYCIVPNDYKLSFGAGTTVTV | |
| | RA | |
| Vα without signal | DAKTTQPPSMDCAEGRAANLPCNHSTISGNEYVYWYRQIHSQGPQYIIHGLK | 1245 |
| peptide (IMGT) | NNETNEMASLIITEDRKSSTLILPHATLRDTAVYYCIVPNDYKLSFGAGTTV | |
| | TVRA | |
| Vα | MXLVARVTVFLTFGTIIDAKTTQPPSMDCAEGRAANLPCNHSTISGNEYVYW | 1246 |
| | YRQIHSQGPQYIIHGLKNNETNEMASLIITEDRKSSTLILPHATLRDTAVYY | 1247 |
| | CIVPNDYKLSFGAGTTVTVRA | |
| | (X = any amino acid) | |
| α chain with WT signal | MRLVARVTVFLTFGTIIDAKTTQPPSMDCAEGRAANLPCNHSTISGNEYVYW | 1248 |
| peptide, Cα | YRQIHSQGPQYIIHGLKNNETNEMASLIITEDRKSSTLILPHATLRDTAVYY | |
| (substituted) | CIVPNDYKLSFGAGTTVTVRANIQNPEPAVYQLKDPRSQDSTLCLFTDFDSQ | |
| | INVPKTMESGTFITDKCVLDMKAMDSKSNGAIAWSNQTSFTCQDIFKETNAT | |
| | YPSSDVPCDATLTEKSFETDMNLNFQNLLVIVLRILLLKVAGFNLLMTLRLW | |
| | SS | |
| α chain with | MALVARVTVFLTFGTIIDAKTTQPPSMDCAEGRAANLPCNHSTISGNEYVYW | 1249 |
| alternative signal | YRQIHSQGPQYIIHGLKNNETNEMASLIITEDRKSSTLILPHATLRDTAVYY | |
| peptide, Cα | CIVPNDYKLSFGAGTTVTVRANIQNPEPAVYQLKDPRSQDSTLCLFTDFDSQ | |
| (substituted) | INVPKTMESGTFITDKCVLDMKAMDSKSNGAIAWSNQTSFTCQDIFKETNAT | |
| | YPSSDVPCDATLTEKSFETDMNLNFQNLLVIVLRILLLKVAGFNLLMTLRLW | |
| | SS | |
| α chain with | MHLVARVTVFLTFGTIIDAKTTQPPSMDCAEGRAANLPCNHSTISGNEYVYW | 1250 |
| alternative signal | YRQIHSQGPQYIIHGLKNNETNEMASLIITEDRKSSTLILPHATLRDTAVYY | |
| peptide, Cα | CIVPNDYKLSFGAGTTVTVRANIQNPEPAVYQLKDPRSQDSTLCLFTDFDSQ | |
| (substituted) | INVPKTMESGTFITDKCVLDMKAMDSKSNGAIAWSNQTSFTCQDIFKETNAT | |
| | YPSSDVPCDATLTEKSFETDMNLNFQNLLVIVLRILLLKVAGFNLLMTLRLW | |
| | SS | |
| CDR1β | MDHEN | 2241 |
| CDR2β | SYDVKM | 2242 |
| CDR3β | ASSFGTGSIQETQY | 2243 |
| Vβ without signal | SRYLVKRTGEKVFLECVQDMDHENMFWYRQDPGLGLRLIYFSYDVKMKEKGD | 2244 |
| peptide (SignalP) | IPEGYSVSREKKERFSLILESASTNQTSMYLCASSFGTGSIQETQYFGPGTR | |
| | LLVL | |
| Vβ without signal | DVKVTQSSRYLVKRTGEKVFLECVQDMDHENMFWYRQDPGLGLRLIYFSYDV | 2245 |
| peptide (IMGT) | KMKEKGDIPEGYSVSREKKERFSLILESASTNQTSMYLCASSFGTGSIQETQ | |
| | YFGPGTRLLVL | |
| Vβ | MXIRLLCRVAFCFLAVGLVDVKVTQSSRYLVKRTGEKVFLECVQDMDHENMF | 2246 |
| | WYRQDPGLGLRLIYFSYDVKMKEKGDIPEGYSVSREKKERFSLILESASTNQ | 2247 |
| | TSMYLCASSFGTGSIQETQYFGPGTRLLVL | |
| | (X = any amino acid) | |
| β chain with WT signal | MGIRLLCRVAFCFLAVGLVDVKVTQSSRYLVKRTGEKVFLECVQDMDHENMF | 2248 |
| peptide, Cβ | WYRQDPGLGLRLIYFSYDVKMKEKGDIPEGYSVSREKKERFSLILESASTNQ | |
| (substituted) | TSMYLCASSFGTGSIQETQYFGPGTRLLVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MAIRLLCRVAFCFLAVGLVDVKVTQSSRYLVKRTGEKVFLECVQDMDHENMF | 2249 |
| signal peptide, Cβ | WYRQDPGLGLRLIYFSYDVKMKEKGDIPEGYSVSREKKERFSLILESASTNQ | |
| (substituted) | TSMYLCASSFGTGSIQETQYFGPGTRLLVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHIRLLCRVAFCFLAVGLVDVKVTQSSRYLVKRTGEKVFLECVQDMDHENMF | 2250 |
| signal peptide, Cβ | WYRQDPGLGLRLIYFSYDVKMKEKGDIPEGYSVSREKKERFSLILESASTNQ | |
| (substituted) | TSMYLCASSFGTGSIQETQYFGPGTRLLVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR025 interacts with and/or is specific for p53. In some embodiments, the peptide is from a neoantigen of p53. In some embodiments, the neoantigen has the amino acid change R248Q relative to the wild type p53 sequence. In some embodiments, TCR025 interacts with the neoantigen in the context of HLA-A*02:01, as described in International Publication No. WO 2019/067243, incorporated herein by reference in its entirety.

TABLE 6Z
Amino acid sequences of TCR026

| | | SEQ |
|---|---|---|
| | | ID |
| Description | Sequence | NO: |
| CDR1α | ATGYPS | 1251 |
| CDR2α | ATKADDK | 1252 |
| CDR3α | ALNPNAGGTSYGKLT | 1253 |
| Vα without signal | NSVTQMEGPVTLSEEAFLTINCTYTATGYPSLFWYVQYPGEGLQLLLKATKA | 1254 |
| peptide (SignalP) | DDKGSNKGFEATYRKETTSFHLEKGSVQVSDSAVYFCALNPNAGGTSYGKLT | |
| | FGQGTILTVHP | |
| Vα without signal | GNSVTQMEGPVTLSEEAFLTINCTYTATGYPSLFWYVQYPGEGLQLLLKATK | 1255 |
| peptide (IMGT) | ADDKGSNKGFEATYRKETTSFHLEKGSVQVSDSAVYFCALNPNAGGTSYGKL | |
| | TFGQGTILTVHP | |
| Vα | MXYSPGLVSLILLLLGRTRGNSVTQMEGPVTLSEEAFLTINCTYTATGYPSL | 1256 |
| | FWYVQYPGEGLQLLLKATKADDKGSNKGFEATYRKETTSFHLEKGSVQVSDS | 1257 |
| | AVYFCALNPNAGGTSYGKLTFGQGTILTVHP | |
| | (X = any amino acid) | |
| α chain with WT signal | MNYSPGLVSLILLLLGRTRGNSVTQMEGPVTLSEEAFLTINCTYTATGYPSL | 1258 |
| peptide, Cα | FWYVQYPGEGLQLLLKATKADDKGSNKGFEATYRKETTSFHLEKGSVQVSDS | |
| (substituted) | AVYFCALNPNAGGTSYGKLTFGQGTILTVHPNIQNPEPAVYQLKDPRSQDST | |
| | LCLFTDFDSQINVPKTMESGTFITDKCVLDMKAMDSKSNGAIAWSNQTSFTC | |
| | QDIFKETNATYPSSDVPCDATLTEKSFETDMNLNFQNLLVIVLRILLLKVAG | |
| | FNLLMTLRLWSS | |
| α chain with | MAYSPGLVSLILLLLGRTRGNSVTQMEGPVTLSEEAFLTINCTYTATGYPSL | 1259 |
| alternative signal | FWYVQYPGEGLQLLLKATKADDKGSNKGFEATYRKETTSFHLEKGSVQVSDS | |
| peptide, Cα | AVYFCALNPNAGGTSYGKLTFGQGTILTVHPNIQNPEPAVYQLKDPRSQDST | |
| (substituted) | LCLFTDFDSQINVPKTMESGTFITDKCVLDMKAMDSKSNGAIAWSNQTSFTC | |
| | QDIFKETNATYPSSDVPCDATLTEKSFETDMNLNFQNLLVIVLRILLLKVAG | |
| | FNLLMTLRLWSS | |
| α chain with | MHYSPGLVSLILLLLGRTRGNSVTQMEGPVTLSEEAFLTINCTYTATGYPSL | 1260 |
| alternative signal | FWYVQYPGEGLQLLLKATKADDKGSNKGFEATYRKETTSFHLEKGSVQVSDS | |
| peptide, Cα | AVYFCALNPNAGGTSYGKLTFGQGTILTVHPNIQNPEPAVYQLKDPRSQDST | |
| (substituted) | LCLFTDFDSQINVPKTMESGTFITDKCVLDMKAMDSKSNGAIAWSNQTSFTC | |
| | QDIFKETNATYPSSDVPCDATLTEKSFETDMNLNFQNLLVIVLRILLLKVAG | |
| | FNLLMTLRLWSS | |
| CDR1β | SGHTS | 2251 |
| CDR2β | YDEGEE | 2252 |
| CDR3β | ASSSPGATSGGANTGELF | 2253 |
| Vβ without signal | GVTQSPTHLIKTRGQQATLRCSPISGHTSVYWYQQALGLGLQFLLWYDEGEE | 2254 |
| peptide (SignalP) | RNRGNFPPRFSGRQFPNYSSELNVNALELEDSALYLCASSSPGATSGGANTG | |
| | ELFFGEGSRLTVL | |
| Vβ without signal | EAGVTQSPTHLIKTRGQQATLRCSPISGHTSVYWYQQALGLGLQFLLWYDEG | 2255 |
| peptide (IMGT) | EERNRGNFPPRFSGRQFPNYSSELNVNALELEDSALYLCASSSPGATSGGAN | |
| | TGELFFGEGSRLTVL | |
| Vβ | MXPRLLFWALLCLLGTGPVEAGVTQSPTHLIKTRGQQATLRCSPISGHTSVY | 2256 |
| | WYQQALGLGLQFLLWYDEGEERNRGNFPPRFSGRQFPNYSSELNVNALELED | 2257 |
| | SALYLCASSSPGATSGGANTGELFFGEGSRLTVL | |
| | (X = any amino acid) | |
| β chain with WT signal | MGPRLLFWALLCLLGTGPVEAGVTQSPTHLIKTRGQQATLRCSPISGHTSVY | 2258 |
| peptide, Cβ | WYQQALGLGLQFLLWYDEGEERNRGNFPPRFSGRQFPNYSSELNVNALELED | |
| (substituted) | SALYLCASSSPGATSGGANTGELFFGEGSRLTVLEDLRNVTPPKVSLFEPSK | |
| | AEIANKQKATLVCLARGFFPDHVELSWWVNGKEVHSGVCTDPQAYKESNYSY | |
| | CLSSRLRVSATFWHNPRNHFRCQVQFHGLSEEDKWPEGSPKPVTQNISAEAW | |
| | GRADCGITSASYQQGVLSATILYEILLGKATLYAVLVSTLVVMAMVKRKNS | |
| β chain with alternative | MAPRLLFWALLCLLGTGPVEAGVTQSPTHLIKTRGQQATLRCSPISGHTSVY | 2259 |
| signal peptide, Cβ | WYQQALGLGLQFLLWYDEGEERNRGNFPPRFSGRQFPNYSSELNVNALELED | |
| (substituted) | SALYLCASSSPGATSGGANTGELFFGEGSRLTVLEDLRNVTPPKVSLFEPSK | |
| | AEIANKQKATLVCLARGFFPDHVELSWWVNGKEVHSGVCTDPQAYKESNYSY | |
| | CLSSRLRVSATFWHNPRNHFRCQVQFHGLSEEDKWPEGSPKPVTQNISAEAW | |
| | GRADCGITSASYQQGVLSATILYEILLGKATLYAVLVSTLVVMAMVKRKNS | |
| β chain with alternative | MHPRLLFWALLCLLGTGPVEAGVTQSPTHLIKTRGQQATLRCSPISGHTSVY | 2260 |
| signal peptide, Cβ | WYQQALGLGLQFLLWYDEGEERNRGNFPPRFSGRQFPNYSSELNVNALELED | |
| (substituted) | SALYLCASSSPGATSGGANTGELFFGEGSRLTVLEDLRNVTPPKVSLFEPSK | |
| | AEIANKQKATLVCLARGFFPDHVELSWWVNGKEVHSGVCTDPQAYKESNYSY | |
| | CLSSRLRVSATFWHNPRNHFRCQVQFHGLSEEDKWPEGSPKPVTQNISAEAW | |
| | GRADCGITSASYQQGVLSATILYEILLGKATLYAVLVSTLVVMAMVKRKNS | |

In some embodiments, TCR026 interacts with and/or is specific for p53. In some embodiments, the peptide is from a neoantigen of p53. In some embodiments, the neoantigen has the amino acid change R248Q relative to the wild type p53 sequence. In some embodiments, TCR026 interacts with the neoantigen in the context of HLA-A*02:01, as described in International Publication No. WO 2019/067243, incorporated herein by reference in its entirety.

TABLE 6AA
Amino acid sequences of TCR027.

| | | SEQ |
|---|---|---|
| | | ID |
| Description | Sequence | NO: |
| CDR1α | DSSSTY | 1261 |
| CDR2α | IFSNMDM | 1262 |
| CDR3α | AEIPRDSGGGADGLT | 1263 |
| Vα without signal | EDVEQSLFLSVREGDSSVINCTYTDSSSTYLYWYKQEPGAGLQLLTYIFSNM | 1264 |
| peptide (SignalP) | DMKQDQRLTVLLNKKDKHLSLRIADTQTGDSAIYFCAEIPRDSGGGADGLTF | |
| | GKGTHLIIQP | |
| Vα without signal | GEDVEQSLFLSVREGDSSVINCTYTDSSSTYLYWYKQEPGAGLQLLTYIFSN | 1265 |
| peptide (IMGT) | MDMKQDQRLTVLLNKKDKHLSLRIADTQTGDSAIYFCAEIPRDSGGGADGLT | |
| | FGKGTHLIIQP | |
| Vα | MXTFAGFSFLFLWLQLDCMSRGEDVEQSLFLSVREGDSSVINCTYTDSSSTY | 1266 |
| | LYWYKQEPGAGLQLLTYIFSNMDMKQDQRLTVLLNKKDKHLSLRIADTQTGD | 1267 |
| | SAIYFCAEIPRDSGGGADGLTFGKGTHLIIQP | |
| | (X = any amino acid) | |
| α chain with WT signal | MKTFAGFSFLFLWLQLDCMSRGEDVEQSLFLSVREGDSSVINCTYTDSSSTY | 1268 |
| peptide, Cα | LYWYKQEPGAGLQLLTYIFSNMDMKQDQRLTVLLNKKDKHLSLRIADTQTGD | |
| (substituted) | SAIYFCAEIPRDSGGGADGLTFGKGTHLIIQPNIQNPEPAVYQLKDPRSQDS | |
| | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| α chain with | MATFAGFSFLFLWLQLDCMSRGEDVEQSLFLSVREGDSSVINCTYTDSSSTY | 1269 |
| alternative signal | LYWYKQEPGAGLQLLTYIFSNMDMKQDQRLTVLLNKKDKHLSLRIADTQTGD | |
| peptide, Cα | SAIYFCAEIPRDSGGGADGLTFGKGTHLIIQPNIQNPEPAVYQLKDPRSQDS | |
| (substituted) | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| α chain with | MHTFAGFSFLFLWLQLDCMSRGEDVEQSLFLSVREGDSSVINCTYTDSSSTY | 1270 |
| alternative signal | LYWYKQEPGAGLQLLTYIFSNMDMKQDQRLTVLLNKKDKHLSLRIADTQTGD | |
| peptide, Cα | SAIYFCAEIPRDSGGGADGLTFGKGTHLIIQPNIQNPEPAVYQLKDPRSQDS | |
| (substituted) | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| CDR1β | DFQATT | 2261 |
| CDR2β | SNEGSKA | 2262 |
| CDR3β | SARDLQRSYEQY | 2263 |
| Vβ without signal | GSGLGAVVSQHPSWVICKSGTSVKIECRSLDFQATTMFWYRQFPKQSLMLMA | 2264 |
| peptide (SignalP) | TSNEGSKATYEQGVEKDKFLINHASLTLSTLTVTSAHPEDSSFYICSARDLQ | |
| | RSYEQYFGPGTRLTVT | |
| Vβ without signal | GAVVSQHPSWVICKSGTSVKIECRSLDFQATTMFWYRQFPKQSLMLMATSNE | 2265 |
| peptide (IMGT) | GSKATYEQGVEKDKFLINHASLTLSTLTVTSAHPEDSSFYICSARDLQRSYE | |
| | QYFGPGTRLTVT | |
| Vβ | MXLLLLLLGPGISLLLPGSLAGSGLGAVVSQHPSWVICKSGTSVKIECRSLD | 2266 |
| | FQATTMFWYRQFPKQSLMLMATSNEGSKATYEQGVEKDKFLINHASLTLSTL | 2267 |
| | TVTSAHPEDSSFYICSARDLQRSYEQYFGPGTRLTVT | |
| | (X = any amino acid) | |
| β chain with WT signal | MLLLLLLLGPGISLLLPGSLAGSGLGAVVSQHPSWVICKSGTSVKIECRSLD | 2268 |
| peptide, Cβ | FQATTMFWYRQFPKQSLMLMATSNEGSKATYEQGVEKDKFLINHASLTLSTL | |
| (substituted) | TVTSAHPEDSSFYICSARDLQRSYEQYFGPGTRLTVTEDLRNVTPPKVSLFE | |
| | PSKAEIANKQKATLVCLARGFFPDHVELSWWVNGKEVHSGVCTDPQAYKESN | |
| | YSYCLSSRLRVSATFWHNPRNHFRCQVQFHGLSEEDKWPEGSPKPVTQNISA | |
| | EAWGRADCGITSASYQQGVLSATILYEILLGKATLYAVLVSTLVVMAMVKRK | |
| | NS | |
| β chain with alternative | MALLLLLLGPGISLLLPGSLAGSGLGAVVSQHPSWVICKSGTSVKIECRSLD | 2269 |
| signal peptide, Cβ | FQATTMFWYRQFPKQSLMLMATSNEGSKATYEQGVEKDKFLINHASLTLSTL | |
| (substituted) | TVTSAHPEDSSFYICSARDLQRSYEQYFGPGTRLTVTEDLRNVTPPKVSLFE | |
| | PSKAEIANKQKATLVCLARGFFPDHVELSWWVNGKEVHSGVCTDPQAYKESN | |
| | YSYCLSSRLRVSATFWHNPRNHFRCQVQFHGLSEEDKWPEGSPKPVTQNISA | |
| | EAWGRADCGITSASYQQGVLSATILYEILLGKATLYAVLVSTLVVMAMVKRK | |
| | NS | |
| β chain with alternative | MHLLLLLLGPGISLLLPGSLAGSGLGAVVSQHPSWVICKSGTSVKIECRSLD | 2270 |
| signal peptide, Cβ | FQATTMFWYRQFPKQSLMLMATSNEGSKATYEQGVEKDKFLINHASLTLSTL | |
| (substituted) | TVTSAHPEDSSFYICSARDLQRSYEQYFGPGTRLTVTEDLRNVTPPKVSLFE | |
| | PSKAEIANKQKATLVCLARGFFPDHVELSWWVNGKEVHSGVCTDPQAYKESN | |
| | YSYCLSSRLRVSATFWHNPRNHFRCQVQFHGLSEEDKWPEGSPKPVTQNISA | |
| | EAWGRADCGITSASYQQGVLSATILYEILLGKATLYAVLVSTLVVMAMVKRK | |
| | NS | |

In some embodiments, TCR027 interacts with and/or is specific for p53. In some embodiments, the peptide is from a neoantigen of p53. In some embodiments, the neoantigen has the amino acid change R248Q relative to the wild type p53 sequence. In some embodiments, TCR27 interacts with the neoantigen in the context of HLA-A*02:01, as described in International Publication No. WO 2019/067243, incorporated herein by reference in its entirety.

TABLE 6AB
Amino acid sequences of TCR028.

| | | SEQ |
|---|---|---|
| | | ID |
| Description | Sequence | NO: |
| CDR1α | DSSSTY | 1271 |
| CDR2α | IFSNMDM | 1272 |
| CDR3α | AEIPRDSGGGADGLT | 1273 |
| Vα without signal | EDVEQSLFLSVREGDSSVINCTYTDSSSTYLYWYKQEPGAGLQLLTYIFSNM | 1274 |
| peptide (SignalP) | DMKQDQRLTVLLNKKDKHLSLRIADTQTGDSAIYFCAEIPRDSGGGADGLTF | |
| | GKGTHLIIQP | |
| Vα without signal | GEDVEQSLFLSVREGDSSVINCTYTDSSSTYLYWYKQEPGAGLQLLTYIFSN | 1275 |
| peptide (IMGT) | MDMKQDQRLTVLLNKKDKHLSLRIADTQTGDSAIYFCAEIPRDSGGGADGLT | |
| | FGKGTHLIIQP | |
| Vα | MXTFAGFSFLFLWLQLDCMSRGEDVEQSLFLSVREGDSSVINCTYTDSSSTY | 1276 |
| | LYWYKQEPGAGLQLLTYIFSNMDMKQDQRLTVLLNKKDKHLSLRIADTQTGD | 1277 |
| | SAIYFCAEIPRDSGGGADGLTFGKGTHLIIQP | |
| | (X = any amino acid) | |
| α chain with WT signal | MKTFAGFSFLFLWLQLDCMSRGEDVEQSLFLSVREGDSSVINCTYTDSSSTY | 1278 |
| peptide, Cα | LYWYKQEPGAGLQLLTYIFSNMDMKQDQRLTVLLNKKDKHLSLRIADTQTGD | |
| (substituted) | SAIYFCAEIPRDSGGGADGLTFGKGTHLIIQPNIQNPEPAVYQLKDPRSQDS | |
| | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| α chain with | MATFAGFSFLFLWLQLDCMSRGEDVEQSLFLSVREGDSSVINCTYTDSSSTY | 1279 |
| alternative signal | LYWYKQEPGAGLQLLTYIFSNMDMKQDQRLTVLLNKKDKHLSLRIADTQTGD | |
| peptide, Cα | SAIYFCAEIPRDSGGGADGLTFGKGTHLIIQPNIQNPEPAVYQLKDPRSQDS | |
| (substituted) | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| α chain with | MHTFAGFSFLFLWLQLDCMSRGEDVEQSLFLSVREGDSSVINCTYTDSSSTY | 1280 |
| alternative signal | LYWYKQEPGAGLQLLTYIFSNMDMKQDQRLTVLLNKKDKHLSLRIADTQTGD | |
| peptide, Cα | SAIYFCAEIPRDSGGGADGLTFGKGTHLIIQPNIQNPEPAVYQLKDPRSQDS | |
| (substituted) | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| CDR1β | DFQATT | 2271 |
| CDR2β | SNEGSKA | 2272 |
| CDR3β | SARDLQRSYEQY | 2273 |
| Vβ without signal | AVVSQHPSRVICKSGTSVKIECRSLDFQATTMFWYRQFPKQSLMLMATSNEG | 2274 |
| peptide (SignalP) | SKATYEQGVEKDKFLINHASLTLSTLTVTSAHPEDSSFYICSARDLQRSYEQ | |
| | YFGPGTRLTVT | |
| Vβ without signal | GAVVSQHPSRVICKSGTSVKIECRSLDFQATTMFWYRQFPKQSLMLMATSNE | 2275 |
| peptide (IMGT) | GSKATYEQGVEKDKFLINHASLTLSTLTVTSAHPEDSSFYICSARDLQRSYE | |
| | QYFGPGTRLTVT | |
| Vβ | MXLLLLLLGPAGSGLGAVVSQHPSRVICKSGTSVKIECRSLDFQATTMFWYR | 2276 |
| | QFPKQSLMLMATSNEGSKATYEQGVEKDKFLINHASLTLSTLTVTSAHPEDS | 2277 |
| | SFYICSARDLQRSYEQYFGPGTRLTVT | |
| | (X = any amino acid) | |
| β chain with WT signal | MLLLLLLLGPAGSGLGAVVSQHPSRVICKSGTSVKIECRSLDFQATTMFWYR | 2278 |
| peptide, Cβ | QFPKQSLMLMATSNEGSKATYEQGVEKDKFLINHASLTLSTLTVTSAHPEDS | |
| (substituted) | SFYICSARDLQRSYEQYFGPGTRLTVTEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain with alternative | MALLLLLLGPAGSGLGAVVSQHPSRVICKSGTSVKIECRSLDFQATTMFWYR | 2279 |
| signal peptide, Cβ | QFPKQSLMLMATSNEGSKATYEQGVEKDKFLINHASLTLSTLTVTSAHPEDS | |
| (substituted) | SFYICSARDLQRSYEQYFGPGTRLTVTEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain with alternative | MHLLLLLLGPAGSGLGAVVSQHPSRVICKSGTSVKIECRSLDFQATTMFWYR | 2280 |
| signal peptide, Cβ | QFPKQSLMLMATSNEGSKATYEQGVEKDKFLINHASLTLSTLTVTSAHPEDS | |
| (substituted) | SFYICSARDLQRSYEQYFGPGTRLTVTEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |

In some embodiments, TCR028 interacts with and/or is specific for p53. In some embodiments, the peptide is from a neoantigen of p53. In some embodiments, the neoantigen has the amino acid change R248Q relative to the wild type p53 sequence. In some embodiments, TCR028 interacts with the neoantigen in the context of HLA-A*02:01, as described in International Publication No. WO 2019/067243, incorporated herein by reference in its entirety.

TABLE 6AC
Amino acid sequences of TCR029.

| | | SEQ |
|---|---|---|
| | | ID |
| Description | Sequence | NO: |
| CDR1α | NSASDY | 1281 |
| CDR2α | IRSNMDK | 1282 |
| CDR3α | AEPVGGLNSGYALN | 1283 |
| Vα without signal | ESVGLHLPTLSVQEGDNSIINCAYSNSASDYFIWYKQESGKGPQFIIDIRSN | 1284 |
| peptide (SignalP) | MDKRQGQRVTVLLNKTVKHLSLQIAATQPGDSAVYFCAEPVGGLNSGYALNF | |
| | GKGTSLLVTP | |
| Vα without signal | GESVGLHLPTLSVQEGDNSIINCAYSNSASDYFIWYKQESGKGPQFIIDIRS | 1285 |
| peptide (IMGT) | NMDKRQGQRVTVLLNKTVKHLSLQIAATQPGDSAVYFCAEPVGGLNSGYALN | |
| | FGKGTSLLVTP | |
| Vα | MXGIRALFMYLWLQLDWVSRGESVGLHLPTLSVQEGDNSIINCAYSNSASDY | 1286 |
| | FIWYKQESGKGPQFIIDIRSNMDKRQGQRVTVLLNKTVKHLSLQIAATQPGD | 1287 |
| | SAVYFCAEPVGGLNSGYALNFGKGTSLLVTP | |
| | (X = any amino acid) | |
| α chain with WT signal | MAGIRALFMYLWLQLDWVSRGESVGLHLPTLSVQEGDNSIINCAYSNSASDY | 1288 |
| peptide, Cα | FIWYKQESGKGPQFIIDIRSNMDKRQGQRVTVLLNKTVKHLSLQIAATQPGD | |
| | SAVYFCAEPVGGLNSGYALNFGKGTSLLVTPNIQNPEPAVYQLKDPRSQDST | |
| (substituted) | LCLFTDFDSQINVPKTMESGTFITDKCVLDMKAMDSKSNGAIAWSNQTSFTC | 1289 |
| | QDIFKETNATYPSSDVPCDATLTEKSFETDMNLNFQNLLVIVLRILLLKVAG | |
| | FNLLMTLRLWSS | |
| α chain with | MHGIRALFMYLWLQLDWVSRGESVGLHLPTLSVQEGDNSIINCAYSNSASDY | 1290 |
| alternative signal | FIWYKQESGKGPQFIIDIRSNMDKRQGQRVTVLLNKTVKHLSLQIAATQPGD | |
| peptide, Cα | SAVYFCAEPVGGLNSGYALNFGKGTSLLVTPNIQNPEPAVYQLKDPRSQDST | |
| (substituted) | LCLFTDFDSQINVPKTMESGTFITDKCVLDMKAMDSKSNGAIAWSNQTSFTC | |
| | QDIFKETNATYPSSDVPCDATLTEKSFETDMNLNFQNLLVIVLRILLLKVAG | |
| | FNLLMTLRLWSS | |
| CDR1β | SGHKS | 2281 |
| CDR2β | QYYEKEE | 2282 |
| CDR3β | ASSGGRTSGAYEQF | 2283 |
| Vβ without signal | GVTQSPTHLIKTRGQQVTLRCSPISGHKSVSWYQQVLGQGPQFIFQYYEKEE | 2284 |
| peptide (SignalP) | RGRGNFPDRFSARQFPNYSSELNVNALLLGDSALYLCASSGGRTSGAYEQFF | |
| | GPGTRLTVL | |
| Vβ without signal | DAGVTQSPTHLIKTRGQQVTLRCSPISGHKSVSWYQQVLGQGPQFIFQYYEK | 2285 |
| peptide (IMGT) | EERGRGNFPDRFSARQFPNYSSELNVNALLLGDSALYLCASSGGRTSGAYEQ | |
| | FFGPGTRLTVL | |
| Vβ | MXPGLLCWVLLCLLGAGPVDAGVTQSPTHLIKTRGQQVTLRCSPISGHKSVS | 2286 |
| | WYQQVLGQGPQFIFQYYEKEERGRGNFPDRESARQFPNYSSELNVNALLLGD | 2287 |
| | SALYLCASSGGRTSGAYEQFFGPGTRLTVL | |
| | (X = any amino acid) | |
| β chain with WT signal | MGPGLLCWVLLCLLGAGPVDAGVTQSPTHLIKTRGQQVTLRCSPISGHKSVS | 2288 |
| peptide, Cβ | WYQQVLGQGPQFIFQYYEKEERGRGNFPDRFSARQFPNYSSELNVNALLLGD | |
| (substituted) | SALYLCASSGGRTSGAYEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MAPGLLCWVLLCLLGAGPVDAGVTQSPTHLIKTRGQQVTLRCSPISGHKSVS | 2289 |
| signal peptide, Cβ | WYQQVLGQGPQFIFQYYEKEERGRGNFPDRFSARQFPNYSSELNVNALLLGD | |
| (substituted) | SALYLCASSGGRTSGAYEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHPGLLCWVLLCLLGAGPVDAGVTQSPTHLIKTRGQQVTLRCSPISGHKSVS | 2290 |
| signal peptide, Cβ | WYQQVLGQGPQFIFQYYEKEERGRGNFPDRFSARQFPNYSSELNVNALLLGD | |
| (substituted) | SALYLCASSGGRTSGAYEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR029 interacts with and/or is specific for p53. In some embodiments, the peptide is from a neoantigen of p53. In some embodiments, the neoantigen has the amino acid change R248Q relative to the wild type p53 sequence. In some embodiments, TCR029 interacts with the neoantigen in the context of HLA-A*02:01, as described in International Publication No. WO 2019/067243, incorporated herein by reference in its entirety.

TABLE 6AD
Amino acid sequences of TCR030.

| | | SEQ |
|---|---|---|
| | | ID |
| Description | Sequence | NO: |
| CDR1α | VSGLRG | 1291 |
| CDR2α | LYSAGEE | 1292 |
| CDR3α | AVTAHRGSTLGRLY | 1293 |
| Vα without signal | EDQVTQSPEALRLQEGESSSLNCSYTVSGLRGLFWYRQDPGKGPEFLFTLYS | 1294 |
| peptide (SignalP) | AGEEKEKERLKATLTKKESFLHITAPKPEDSATYLCAVTAHRGSTLGRLYFG | |
| | RGTQLTVWP | |
| Vα without signal | EDQVTQSPEALRLQEGESSSLNCSYTVSGLRGLFWYRQDPGKGPEFLFTLYS | 1295 |
| peptide (IMGT) | AGEEKEKERLKATLTKKESFLHITAPKPEDSATYLCAVTAHRGSTLGRLYFG | |
| | RGTQLTVWP | |
| Vα | MXKMLECAFIVLWLQLGWLSGEDQVTQSPEALRLQEGESSSLNCSYTVSGLR | 1296 |
| | GLFWYRQDPGKGPEFLFTLYSAGEEKEKERLKATLTKKESFLHITAPKPEDS | 1297 |
| | ATYLCAVTAHRGSTLGRLYFGRGTQLTVWP | |
| | (X = any amino acid) | |
| α chain with WT signal | MEKMLECAFIVLWLQLGWLSGEDQVTQSPEALRLQEGESSSLNCSYTVSGLR | 1298 |
| peptide, Cα | GLFWYRQDPGKGPEFLFTLYSAGEEKEKERLKATLTKKESFLHITAPKPEDS | |
| (substituted) | ATYLCAVTAHRGSTLGRLYFGRGTQLTVWPNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MAKMLECAFIVLWLQLGWLSGEDQVTQSPEALRLQEGESSSLNCSYTVSGLR | 1299 |
| alternative signal | GLFWYRQDPGKGPEFLFTLYSAGEEKEKERLKATLTKKESFLHITAPKPEDS | |
| peptide, Cα | ATYLCAVTAHRGSTLGRLYFGRGTQLTVWPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MHKMLECAFIVLWLQLGWLSGEDQVTQSPEALRLQEGESSSLNCSYTVSGLR | 1300 |
| alternative signal | GLFWYRQDPGKGPEFLFTLYSAGEEKEKERLKATLTKKESFLHITAPKPEDS | |
| peptide, Cα | ATYLCAVTAHRGSTLGRLYFGRGTQLTVWPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| CDR1β | SGHDT | 2291 |
| CDR2β | YYEEEE | 2292 |
| CDR3β | ASSRRGGAYNEQF | 2293 |
| Vβ without signal | GVTQSPTHLIKTRGQQVTLRCSPKSGHDTVSWYQQALGQGPQFIFQYYEEEE | 2294 |
| peptide (SignalP) | RQRGNFPDRFSGHQFPNYSSELNVNALLLGDSALYLCASSRRGGAYNEQFFG | |
| | PGTRLTVL | |
| Vβ without signal | DAGVTQSPTHLIKTRGQQVTLRCSPKSGHDTVSWYQQALGQGPQFIFQYYEE | 2295 |
| peptide (IMGT) | EERQRGNFPDRFSGHQFPNYSSELNVNALLLGDSALYLCASSRRGGAYNEQF | |
| | FGPGTRLTVL | |
| Vβ | MXPGLLCWALLCLLGAGLVDAGVTQSPTHLIKTRGQQVTLRCSPKSGHDTVS | 2296 |
| | WYQQALGQGPQFIFQYYEEEERQRGNFPDRFSGHQFPNYSSELNVNALLLGD | 2297 |
| | SALYLCASSRRGGAYNEQFFGPGTRLTVL | |
| | (X = any amino acid) | |
| β chain with WT signal | MGPGLLCWALLCLLGAGLVDAGVTQSPTHLIKTRGQQVTLRCSPKSGHDTVS | 2298 |
| peptide, Cβ | WYQQALGQGPQFIFQYYEEEERQRGNFPDRFSGHQFPNYSSELNVNALLLGD | |
| (substituted) | SALYLCASSRRGGAYNEQFFGPGTRLTVLEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain with alternative | MAPGLLCWALLCLLGAGLVDAGVTQSPTHLIKTRGQQVTLRCSPKSGHDTVS | 2299 |
| signal peptide, Cβ | WYQQALGQGPQFIFQYYEEEERQRGNFPDRFSGHQFPNYSSELNVNALLLGD | |
| (substituted) | SALYLCASSRRGGAYNEQFFGPGTRLTVLEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain with alternative | MHPGLLCWALLCLLGAGLVDAGVTQSPTHLIKTRGQQVTLRCSPKSGHDTVS | 2300 |
| signal peptide, Cβ | WYQQALGQGPQFIFQYYEEEERQRGNFPDRFSGHQFPNYSSELNVNALLLGD | |
| (substituted) | SALYLCASSRRGGAYNEQFFGPGTRLTVLEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR030 interacts with and/or is specific for p53. In some embodiments, the peptide is from a neoantigen of p53. In some embodiments, the neoantigen has the amino acid change R248Q relative to the wild type p53 sequence. In some embodiments, TCR030 interacts with the neoantigen in the context of HLA-A*02:01, as described in International Publication No. WO 2019/067243, incorporated herein by reference in its entirety.

TABLE 6AE
Amino acid sequences of TCR031.

| | | SEQ |
|---|---|---|
| | | ID |
| Description | Sequence | NO: |
| CDR1α | SSNFYA | 1301 |
| CDR2α | MTLNGDE | 1302 |
| CDR3α | ASVGGGADGLT | 1303 |
| Vα without signal | ILNVEQSPQSLHVQEGDSTNFTCSFPSSNFYALHWYRWETAKSPEALFVMTL | 1304 |
| peptide (SignalP) | NGDEKKKGRISATLNTKEGYSYLYIKGSQPEDSATYLCASVGGGADGLTFGK | |
| | GTHLIIQP | |
| Vα without signal | ILNVEQSPQSLHVQEGDSTNFTCSFPSSNFYALHWYRWETAKSPEALFVMTL | 1305 |
| peptide (IMGT) | NGDEKKKGRISATLNTKEGYSYLYIKGSQPEDSATYLCASVGGGADGLTFGK | |
| | GTHLIIQP | |
| Vα | MXKNPLAAPLLILWFHLDCVSSILNVEQSPQSLHVQEGDSTNFTCSFPSSNF | 1306 |
| | YALHWYRWETAKSPEALFVMTLNGDEKKKGRISATLNTKEGYSYLYIKGSQP | 1307 |
| | EDSATYLCASVGGGADGLTFGKGTHLIIQP | |
| | (X = any amino acid) | |
| α chain with WT signal | MEKNPLAAPLLILWFHLDCVSSILNVEQSPQSLHVQEGDSTNFTCSFPSSNF | 1308 |
| peptide, Cα | YALHWYRWETAKSPEALFVMTLNGDEKKKGRISATLNTKEGYSYLYIKGSQP | |
| (substituted) | EDSATYLCASVGGGADGLTFGKGTHLIIQPNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MAKNPLAAPLLILWFHLDCVSSILNVEQSPQSLHVQEGDSTNFTCSFPSSNF | 1309 |
| alternative signal | YALHWYRWETAKSPEALFVMTLNGDEKKKGRISATLNTKEGYSYLYIKGSQP | |
| peptide, Cα | EDSATYLCASVGGGADGLTFGKGTHLIIQPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MHKNPLAAPLLILWFHLDCVSSILNVEQSPQSLHVQEGDSTNFTCSFPSSNF | 1310 |
| alternative signal | YALHWYRWETAKSPEALFVMTLNGDEKKKGRISATLNTKEGYSYLYIKGSQP | |
| peptide, Cα | EDSATYLCASVGGGADGLTFGKGTHLIIQPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| CDR1β | SGHTA | 2301 |
| CDR2β | FQGNSA | 2302 |
| CDR3β | ASTWDRGSYNEQF | 2303 |
| Vβ without signal | GVSQSPSNKVTEKGKDVELRCDPISGHTALYWYRQSLGQGLEFLIYFQGNSA | 2304 |
| peptide (SignalP) | PDKSGLPSDRFSAERTGGSVSTLTIQRTQQEDSAVYLCASTWDRGSYNEQFF | |
| | GPGTRLTVL | |
| Vβ without signal | GAGVSQSPSNKVTEKGKDVELRCDPISGHTALYWYRQSLGQGLEFLIYFQGN | 2305 |
| peptide (IMGT) | SAPDKSGLPSDRFSAERTGGSVSTLTIQRTQQEDSAVYLCASTWDRGSYNEQ | |
| | FFGPGTRLTVL | |
| Vβ | MXTRLLFWVAFCLLGADHTGAGVSQSPSNKVTEKGKDVELRCDPISGHTALY | 2306 |
| | WYRQSLGQGLEFLIYFQGNSAPDKSGLPSDRFSAERTGGSVSTLTIQRTQQE | 2307 |
| | DSAVYLCASTWDRGSYNEQFFGPGTRLTVL | |
| | (X = any amino acid) | |
| β chain with WT signal | MGTRLLFWVAFCLLGADHTGAGVSQSPSNKVTEKGKDVELRCDPISGHTALY | 2308 |
| peptide, Cβ | WYRQSLGQGLEFLIYFQGNSAPDKSGLPSDRESAERTGGSVSTLTIQRTQQE | |
| (substituted) | DSAVYLCASTWDRGSYNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLFWVAFCLLGADHTGAGVSQSPSNKVTEKGKDVELRCDPISGHTALY | 2309 |
| signal peptide, Cβ | WYRQSLGQGLEFLIYFQGNSAPDKSGLPSDRESAERTGGSVSTLTIQRTQQE | |
| (substituted) | DSAVYLCASTWDRGSYNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLFWVAFCLLGADHTGAGVSQSPSNKVTEKGKDVELRCDPISGHTALY | 2310 |
| signal peptide, Cβ | WYRQSLGQGLEFLIYFQGNSAPDKSGLPSDRFSAERTGGSVSTLTIQRTQQE | |
| (substituted) | DSAVYLCASTWDRGSYNEQFFGPGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR031 interacts with and/or is specific for p53. In some embodiments, the peptide is from a neoantigen of p53. In some embodiments, the neoantigen has the amino acid change R248Q relative to the wild type p53 sequence. In some embodiments, TCR031 interacts with the neoantigen in the context of HLA-A*02:01, as described in International Publication No. WO 2019/067243, incorporated herein by reference in its entirety.

TABLE 6AF
Amino acid sequences of TCR032.

| | | SEQ |
|---|---|---|
| | | ID |
| Description | Sequence | NO: |
| CDR1α | NSMEDY | 1311 |
| CDR2α | ISSIKDK | 1312 |
| CDR3α | AANTGNQFY | 1313 |
| Vα without signal | QQKNDDQQVKQNSPSLSVQEGRISILNCDYTNSMFDYFLWYKKYPAEGPTFL | 1314 |
| peptide (SignalP) | ISISSIKDKNEDGRFTVFLNKSAKHLSLHIVPSQPGDSAVYFCAANTGNQFY | |
| | FGTGTSLTVIP | |
| Vα without signal | DQQVKQNSPSLSVQEGRISILNCDYTNSMFDYFLWYKKYPAEGPTFLISISS | 1315 |
| peptide (IMGT) | IKDKNEDGRFTVFLNKSAKHLSLHIVPSQPGDSAVYFCAANTGNQFYFGTGT | |
| | SLTVIP | |
| Vα | MXMLLGASVLILWLQPDWVNSQQKNDDQQVKQNSPSLSVQEGRISILNCDYT | 1316 |
| | NSMFDYFLWYKKYPAEGPTFLISISSIKDKNEDGRFTVFLNKSAKHLSLHIV | |
| | PSQPGDSAVYFCAANTGNQFYFGTGTSLTVIP | 1317 |
| | (X = any amino acid) | |
| α chain with WT signal | MAMLLGASVLILWLQPDWVNSQQKNDDQQVKQNSPSLSVQEGRISILNCDYT | 1318 |
| peptide, Cα | NSMFDYFLWYKKYPAEGPTFLISISSIKDKNEDGRFTVFLNKSAKHLSLHIV | 1319 |
| (substituted) | PSQPGDSAVYFCAANTGNQFYFGTGTSLTVIPNIQNPEPAVYQLKDPRSQDS | |
| | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| α chain with | MHMLLGASVLILWLQPDWVNSQQKNDDQQVKQNSPSLSVQEGRISILNCDYT | 1320 |
| alternative signal | NSMFDYFLWYKKYPAEGPTFLISISSIKDKNEDGRFTVFLNKSAKHLSLHIV | |
| peptide, Cα | PSQPGDSAVYFCAANTGNQFYFGTGTSLTVIPNIQNPEPAVYQLKDPRSQDS | |
| (substituted) | TLCLFTDFDSQINVPKTMESGTFITDKCVLDMKAMDSKSNGAIAWSNQTSFT | |
| | CQDIFKETNATYPSSDVPCDATLTEKSFETDMNLNFQNLLVIVLRILLLKVA | |
| | GFNLLMTLRLWSS | |
| CDR1β | SGHAT | 2311 |
| CDR2β | FQNNGV | 2312 |
| CDR3β | ASSHLAGEFYNEQF | 2313 |
| Vβ without signal | GVAQSPRYKIIEKRQSVAFWCNPISGHATLYWYQQILGQGPKLLIQFQNNGV | 2314 |
| peptide (SignalP) | VDDSQLPKDRFSAERLKGVDSTLKIQPAKLEDSAVYLCASSHLAGEFYNEQF | |
| | FGPGTRLTVL | |
| Vβ without signal | EAGVAQSPRYKIIEKRQSVAFWCNPISGHATLYWYQQILGQGPKLLIQFQNN | 2315 |
| peptide (IMGT) | GVVDDSQLPKDRFSAERLKGVDSTLKIQPAKLEDSAVYLCASSHLAGEFYNE | |
| | QFFGPGTRLTVL | |
| Vβ | MXTRLLCWAALCLLGAELTEAGVAQSPRYKIIEKRQSVAFWCNPISGHATLY | 2316 |
| | WYQQILGQGPKLLIQFQNNGVVDDSQLPKDRFSAERLKGVDSTLKIQPAKLE | 2317 |
| | DSAVYLCASSHLAGEFYNEQFFGPGTRLTVL | |
| | (X = any amino acid) | |
| β chain with WT signal | MGTRLLCWAALCLLGAELTEAGVAQSPRYKIIEKRQSVAFWCNPISGHATLY | 2318 |
| peptide, Cβ | WYQQILGQGPKLLIQFQNNGVVDDSQLPKDRFSAERLKGVDSTLKIQPAKLE | |
| (substituted) | DSAVYLCASSHLAGEFYNEQFFGPGTRLTVLEDLRNVTPPKVSLFEPSKAEI | |
| | ANKQKATLVCLARGFFPDHVELSWWVNGKEVHSGVCTDPQAYKESNYSYCLS | |
| | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |
| β chain with alternative | MATRLLCWAALCLLGAELTEAGVAQSPRYKIIEKRQSVAFWCNPISGHATLY | 2319 |
| signal peptide, Cβ | WYQQILGQGPKLLIQFQNNGVVDDSQLPKDRFSAERLKGVDSTLKIQPAKLE | |
| (substituted) | DSAVYLCASSHLAGEFYNEQFFGPGTRLTVLEDLRNVTPPKVSLFEPSKAEI | |
| | ANKQKATLVCLARGFFPDHVELSWWVNGKEVHSGVCTDPQAYKESNYSYCLS | |
| | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |
| β chain with alternative | MHTRLLCWAALCLLGAELTEAGVAQSPRYKIIEKRQSVAFWCNPISGHATLY | 2320 |
| signal peptide, Cβ | WYQQILGQGPKLLIQFQNNGVVDDSQLPKDRFSAERLKGVDSTLKIQPAKLE | |
| (substituted) | DSAVYLCASSHLAGEFYNEQFFGPGTRLTVLEDLRNVTPPKVSLFEPSKAEI | |
| | ANKQKATLVCLARGFFPDHVELSWWVNGKEVHSGVCTDPQAYKESNYSYCLS | |
| | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |

In some embodiments, TCR032 interacts with and/or is specific for p53. In some embodiments, the peptide is from a neoantigen of p53. In some embodiments, the neoantigen has the amino acid change R248Q relative to the wild type p53 sequence. In some embodiments, TCR032 interacts with the neoantigen in the context of HLA-A*02:01, as described in International Publication No. WO 2019/067243, incorporated herein by reference in its entirety.

TABLE 6AG
Amino acid sequences of TCR033.

| | | SEQ |
|---|---|---|
| | | ID |
| Description | Sequence | NO: |
| CDR1α | TSGFYG | 1321 |
| CDR2α | NALDGL | 1322 |
| CDR3α | AFAYGQNFV | 1323 |
| Vα without signal | QSLEQPSEVTAVEGAIVQINCTYQTSGFYGLSWYQQHDGGAPTFLSYNALDG | 1324 |
| peptide (SignalP) | LEETGRFSSFLSRSDSYGYLLLQELQMKDSASYFCAFAYGQNFVFGPGTRLS | |
| | VLP | |
| Vα without signal | GQSLEQPSEVTAVEGAIVQINCTYQTSGFYGLSWYQQHDGGAPTFLSYNALD | 1325 |
| peptide (IMGT) | GLEETGRFSSFLSRSDSYGYLLLQELQMKDSASYFCAFAYGQNFVFGPGTRL | |
| | SVLP | |
| Vα | MXGAFLLYVSMKMGGTAGQSLEQPSEVTAVEGAIVQINCTYQTSGFYGLSWY | 1326 |
| | QQHDGGAPTFLSYNALDGLEETGRFSSFLSRSDSYGYLLLQELQMKDSASYF | 1327 |
| | CAFAYGQNFVFGPGTRLSVLP | |
| | (X = any amino acid) | |
| α chain with WT signal | MWGAFLLYVSMKMGGTAGQSLEQPSEVTAVEGAIVQINCTYQTSGFYGLSWY | 1328 |
| peptide, Cα | QQHDGGAPTFLSYNALDGLEETGRFSSFLSRSDSYGYLLLQELQMKDSASYF | |
| (substituted) | CAFAYGQNFVFGPGTRLSVLPNIQNPEPAVYQLKDPRSQDSTLCLFTDFDSQ | |
| | INVPKTMESGTFITDKCVLDMKAMDSKSNGAIAWSNQTSFTCQDIFKETNAT | |
| | YPSSDVPCDATLTEKSFETDMNLNFQNLLVIVLRILLLKVAGFNLLMTLRLW | |
| | SS | |
| α chain with | MAGAFLLYVSMKMGGTAGQSLEQPSEVTAVEGAIVQINCTYQTSGFYGLSWY | 1329 |
| alternative signal | QQHDGGAPTFLSYNALDGLEETGRFSSFLSRSDSYGYLLLQELQMKDSASYF | |
| peptide, Cα | CAFAYGQNFVFGPGTRLSVLPNIQNPEPAVYQLKDPRSQDSTLCLFTDFDSQ | |
| (substituted) | INVPKTMESGTFITDKCVLDMKAMDSKSNGAIAWSNQTSFTCQDIFKETNAT | |
| | YPSSDVPCDATLTEKSFETDMNLNFQNLLVIVLRILLLKVAGFNLLMTLRLW | |
| | SS | |
| α chain with | MHGAFLLYVSMKMGGTAGQSLEQPSEVTAVEGAIVQINCTYQTSGFYGLSWY | 1330 |
| alternative signal | QQHDGGAPTFLSYNALDGLEETGRFSSFLSRSDSYGYLLLQELQMKDSASYF | |
| peptide, Cα | CAFAYGQNFVFGPGTRLSVLPNIQNPEPAVYQLKDPRSQDSTLCLFTDFDSQ | |
| (substituted) | INVPKTMESGTFITDKCVLDMKAMDSKSNGAIAWSNQTSFTCQDIFKETNAT | |
| | YPSSDVPCDATLTEKSFETDMNLNFQNLLVIVLRILLLKVAGFNLLMTLRLW | |
| | SS | |
| CDR1β | SGDLS | 2321 |
| CDR2β | YYNGEE | 2322 |
| CDR3β | ASSPLGDSGNTIY | 2323 |
| Vβ without signal | GVTQTPKHLITATGQRVTLRCSPRSGDLSVYWYQQSLDQGLQFLIQYYNGEE | 2324 |
| peptide (SignalP) | RAKGNILERFSAQQFPDLHSELNLSSLELGDSALYFCASSPLGDSGNTIYFG | |
| | EGSWLTVV | |
| Vβ without signal | DSGVTQTPKHLITATGQRVTLRCSPRSGDLSVYWYQQSLDQGLQFLIQYYNG | 2325 |
| peptide (IMGT) | EERAKGNILERFSAQQFPDLHSELNLSSLELGDSALYFCASSPLGDSGNTIY | |
| | FGEGSWLTVV | |
| Vβ | MXFRLLCCVAFCLLGAGPVDSGVTQTPKHLITATGQRVTLRCSPRSGDLSVY | 2326 |
| | WYQQSLDQGLQFLIQYYNGEERAKGNILERFSAQQFPDLHSELNLSSLELGD | 2327 |
| | SALYFCASSPLGDSGNTIYFGEGSWLTVV | |
| | (X = any amino acid) | |
| β chain with WT signal | MGFRLLCCVAFCLLGAGPVDSGVTQTPKHLITATGQRVTLRCSPRSGDLSVY | 2328 |
| peptide, Cβ | WYQQSLDQGLQFLIQYYNGEERAKGNILERFSAQQFPDLHSELNLSSLELGD | |
| (substituted) | SALYFCASSPLGDSGNTIYFGEGSWLTVVEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain with alternative | MAFRLLCCVAFCLLGAGPVDSGVTQTPKHLITATGQRVTLRCSPRSGDLSVY | 2329 |
| signal peptide, Cβ | WYQQSLDQGLQFLIQYYNGEERAKGNILERFSAQQFPDLHSELNLSSLELGD | |
| (substituted) | SALYFCASSPLGDSGNTIYFGEGSWLTVVEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain with alternative | MHFRLLCCVAFCLLGAGPVDSGVTQTPKHLITATGQRVTLRCSPRSGDLSVY | 2330 |
| signal peptide, Cβ | WYQQSLDQGLQFLIQYYNGEERAKGNILERFSAQQFPDLHSELNLSSLELGD | |
| (substituted) | SALYFCASSPLGDSGNTIYFGEGSWLTVVEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR034 interacts with and/or is specific for p53. In some embodiments, the peptide is from a neoantigen of p53. In some embodiments, the neoantigen has the amino acid change R248Q relative to the wild type p53 sequence. In some embodiments, TCR034 interacts with the neoantigen in the context of HLA-A*02:01, as described in International Publication No. WO 2019/067243, incorporated herein by reference in its entirety.

TABLE 6AH
Amino acid sequences of TCR034.

| | | SEQ |
|---|---|---|
| | | ID |
| Description | Sequence | NO: |
| CDR1α | TSINN | 1331 |
| CDR2α | IRSNERE | 1332 |
| CDR3α | ATDAWNNDMR | 1333 |
| Vα without signal | QQGEEDPQALSIQEGENATMNCSYKTSINNLQWYRQNSGRGLVHLILIRSNE | 1334 |
| peptide (SignalP) | REKHSGRLRVTLDTSKKSSSLLITASRAADTASYFCATDAWNNDMRFGAGTR | |
| | LTVKP | |
| Vα without signal | SQQGEEDPQALSIQEGENATMNCSYKTSINNLQWYRQNSGRGLVHLILIRSN | 1335 |
| peptide (IMGT) | EREKHSGRLRVTLDTSKKSSSLLITASRAADTASYFCATDAWNNDMRFGAGT | |
| | RLTVKP | |
| Vα | MXTLLGVSLVILWLQLARVNSQQGEEDPQALSIQEGENATMNCSYKTSINNL | 1336 |
| | QWYRQNSGRGLVHLILIRSNEREKHSGRLRVTLDTSKKSSSLLITASRAADT | 1337 |
| | ASYFCATDAWNNDMRFGAGTRLTVKP | |
| | (X = any amino acid) | |
| α chain with WT signal | METLLGVSLVILWLQLARVNSQQGEEDPQALSIQEGENATMNCSYKTSINNL | 1338 |
| peptide, Cα | QWYRQNSGRGLVHLILIRSNEREKHSGRLRVTLDTSKKSSSLLITASRAADT | |
| (substituted) | ASYFCATDAWNNDMRFGAGTRLTVKPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain with | MATLLGVSLVILWLQLARVNSQQGEEDPQALSIQEGENATMNCSYKTSINNL | 1339 |
| alternative signal | QWYRQNSGRGLVHLILIRSNEREKHSGRLRVTLDTSKKSSSLLITASRAADT | |
| peptide, Cα | ASYFCATDAWNNDMRFGAGTRLTVKPNIQNPEPAVYQLKDPRSQDSTLCLFT | |
| (substituted) | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain with | MHTLLGVSLVILWLQLARVNSQQGEEDPQALSIQEGENATMNCSYKTSINNL | 1340 |
| alternative signal | QWYRQNSGRGLVHLILIRSNEREKHSGRLRVTLDTSKKSSSLLITASRAADT | |
| peptide, Cα | ASYFCATDAWNNDMRFGAGTRLTVKPNIQNPEPAVYQLKDPRSQDSTLCLFT | |
| (substituted) | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| CDR1β | MNHNS | 2331 |
| CDR2β | SASEG | 2332 |
| CDR3β | ASSESQGNTEAF | 2333 |
| Vβ without signal | GVTQTPKFQVLKTGQSMTLQCAQDMNHNSMYWYRQDPGMGLRLIYYSASEGT | 2334 |
| peptide (SignalP) | TDKGEVPNGYNVSRLNKREFSLRLESAAPSQTSVYFCASSESQGNTEAFFGQ | |
| | GTRLTVV | |
| Vβ without signal | NAGVTQTPKFQVLKTGQSMTLQCAQDMNHNSMYWYRQDPGMGLRLIYYSASE | 2335 |
| peptide (IMGT) | GTTDKGEVPNGYNVSRLNKREFSLRLESAAPSQTSVYFCASSESQGNTEAFF | |
| | GQGTRLTVV | |
| Vβ | MXIGLLCCVAFSLLWASPVNAGVTQTPKFQVLKTGQSMTLQCAQDMNHNSMY | 2336 |
| | WYRQDPGMGLRLIYYSASEGTTDKGEVPNGYNVSRLNKREFSLRLESAAPSQ | 2337 |
| | TSVYFCASSESQGNTEAFFGQGTRLTVV | |
| | (X = any amino acid) | |
| β chain with WT signal | MSIGLLCCVAFSLLWASPVNAGVTQTPKFQVLKTGQSMTLQCAQDMNHNSMY | 2338 |
| peptide, Cβ | WYRQDPGMGLRLIYYSASEGTTDKGEVPNGYNVSRLNKREFSLRLESAAPSQ | |
| (substituted) | TSVYFCASSESQGNTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MAIGLLCCVAFSLLWASPVNAGVTQTPKFQVLKTGQSMTLQCAQDMNHNSMY | 2339 |
| signal peptide, Cβ | WYRQDPGMGLRLIYYSASEGTTDKGEVPNGYNVSRLNKREFSLRLESAAPSQ | |
| (substituted) | TSVYFCASSESQGNTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with WT signal | MHIGLLCCVAFSLLWASPVNAGVTQTPKFQVLKTGQSMTLQCAQDMNHNSMY | 2340 |
| peptide, Cβ | WYRQDPGMGLRLIYYSASEGTTDKGEVPNGYNVSRLNKREFSLRLESAAPSQ | |
| (substituted) | TSVYFCASSESQGNTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR034 interacts with and/or is specific for p53. In some embodiments, the peptide is from a neoantigen of p53. In some embodiments, the neoantigen has the amino acid change R248Q relative to the wild type p53 sequence. In some embodiments, TCR034 interacts with the neoantigen in the context of HLA-A*02:01, as described in International Publication No. WO 2019/067243, incorporated herein by reference in its entirety.

TABLE 6AI
Amino acid sequences of TCR035.

| | | SEQ |
|---|---|---|
| | | ID |
| Description | Sequence | NO: |
| CDR1α | VSPFSN | 1341 |
| CDR2α | MTFSENT | 1342 |
| CDR3α | VVSSYKII | 1343 |
| Vα without signal | KNQVEQSPQSLIILEGKNCTLQCNYTVSPFSNLRWYKQDTGRGPVSLTIMTF | 1344 |
| peptide (SignalP) | SENTKSNGRYTATLDADTKQSSLHITASQLSDSASYICVVSSYKIIFGTGTR | |
| | LHVFP | |
| Vα without signal | KNQVEQSPQSLIILEGKNCTLQCNYTVSPFSNLRWYKQDTGRGPVSLTIMTF | 1345 |
| peptide (IMGT) | SENTKSNGRYTATLDADTKQSSLHITASQLSDSASYICVVSSYKIIFGTGTR | |
| | LHVFP | |
| Vα | MXKHLTTFLVILWLYFYRGNGKNQVEQSPQSLIILEGKNCTLQCNYTVSPFS | 1346 |
| | NLRWYKQDTGRGPVSLTIMTFSENTKSNGRYTATLDADTKQSSLHITASQLS | 1347 |
| | DSASYICVVSSYKIIFGTGTRLHVFP | |
| | (X = any amino acid) | |
| α chain with WT signal | MKKHLTTFLVILWLYFYRGNGKNQVEQSPQSLIILEGKNCTLQCNYTVSPFS | 1348 |
| peptide, Cα | NLRWYKQDTGRGPVSLTIMTFSENTKSNGRYTATLDADTKQSSLHITASQLS | |
| (substituted) | DSASYICVVSSYKIIFGTGTRLHVFPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain with | MAKHLTTFLVILWLYFYRGNGKNQVEQSPQSLIILEGKNCTLQCNYTVSPFS | 1349 |
| alternative signal | NLRWYKQDTGRGPVSLTIMTFSENTKSNGRYTATLDADTKQSSLHITASQLS | |
| peptide, Cα | DSASYICVVSSYKIIFGTGTRLHVFPNIQNPEPAVYQLKDPRSQDSTLCLFT | |
| (substituted) | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain with | MHKHLTTFLVILWLYFYRGNGKNQVEQSPQSLIILEGKNCTLQCNYTVSPFS | 1350 |
| alternative signal | NLRWYKQDTGRGPVSLTIMTFSENTKSNGRYTATLDADTKQSSLHITASQLS | |
| peptide, Cα | DSASYICVVSSYKIIFGTGTRLHVFPNIQNPEPAVYQLKDPRSQDSTLCLFT | |
| (substituted) | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| CDR1β | SGHTA | 2341 |
| CDR2β | FQGNSA | 2342 |
| CDR3β | ASSPIQGENSPLH | 2343 |
| Vβ without signal | GVSQSPSNKVTEKGKDVELRCDPISGHTALYWYRQRLGQGLEFLIYFQGNSA | 2344 |
| peptide (SignalP) | PDKSGLPSDRFSAERTGESVSTLTIQRTQQEDSAVYLCASSPIQGENSPLHF | |
| | GNGTRLTVT | |
| Vβ without signal | GAGVSQSPSNKVTEKGKDVELRCDPISGHTALYWYRQRLGQGLEFLIYFQGN | 2345 |
| peptide (IMGT) | SAPDKSGLPSDRESAERTGESVSTLTIQRTQQEDSAVYLCASSPIQGENSPL | |
| | HFGNGTRLTVT | |
| Vβ | MXTRLLFWVAFCLLGAYHTGAGVSQSPSNKVTEKGKDVELRCDPISGHTALY | 2346 |
| | WYRQRLGQGLEFLIYFQGNSAPDKSGLPSDRESAERTGESVSTLTIQRTQQE | 2347 |
| | DSAVYLCASSPIQGENSPLHFGNGTRLTVT | |
| | (X = any amino acid) | |
| β chain with WT signal | MGTRLLFWVAFCLLGAYHTGAGVSQSPSNKVTEKGKDVELRCDPISGHTALY | 2348 |
| peptide, Cβ | WYRQRLGQGLEFLIYFQGNSAPDKSGLPSDRFSAERTGESVSTLTIQRTQQE | |
| (substituted) | DSAVYLCASSPIQGENSPLHFGNGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLFWVAFCLLGAYHTGAGVSQSPSNKVTEKGKDVELRCDPISGHTALY | 2349 |
| signal peptide, Cβ | WYRQRLGQGLEFLIYFQGNSAPDKSGLPSDRFSAERTGESVSTLTIQRTQQE | |
| (substituted) | DSAVYLCASSPIQGENSPLHFGNGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLFWVAFCLLGAYHTGAGVSQSPSNKVTEKGKDVELRCDPISGHTALY | 2350 |
| signal peptide, Cβ | WYRQRLGQGLEFLIYFQGNSAPDKSGLPSDRFSAERTGESVSTLTIQRTQQE | |
| (substituted) | DSAVYLCASSPIQGENSPLHFGNGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR035 interacts with and/or is specific for p53. In some embodiments, the peptide is from a neoantigen of p53. In some embodiments, the neoantigen has the amino acid change R248Q relative to the wild type p53 sequence. In some embodiments, TCR035 interacts with the neoantigen in the context of HLA-A*02:01, as described in International Publication No. WO 2019/067243, incorporated herein by reference in its entirety.

TABLE 6AJ
Amino acid sequences of TCR036.

| | | SEQ |
|---|---|---|
| | | ID |
| Description | Sequence | NO: |
| CDR1α | VSPFSN | 1351 |
| CDR2α | MTFSENT | 1352 |
| CDR3α | VVSSYKLI | 1353 |
| Vα without signal | KNQVEQSPQSLIILEGKNCTLQCNYTVSPFSNLRWYKQDTGRGPVSLTIMTF | 1354 |
| peptide (SignalP) | SENTKSNGRYTATLDADTKQSSLHITASQLSDSASYICVVSSYKLIFGTGTR | |
| | LQVFP | |
| Vα without signal | KNQVEQSPQSLIILEGKNCTLQCNYTVSPFSNLRWYKQDTGRGPVSLTIMTE | 1355 |
| peptide (IMGT) | SENTKSNGRYTATLDADTKQSSLHITASQLSDSASYICVVSSYKLIFGTGTR | |
| | LQVFP | |
| Vα | MXKHLTTFLVILWLYFYRGNGKNQVEQSPQSLIILEGKNCTLQCNYTVSPFS | 1356 |
| | NLRWYKQDTGRGPVSLTIMTFSENTKSNGRYTATLDADTKQSSLHITASQLS | 1357 |
| | DSASYICVVSSYKLIFGTGTRLQVFP | |
| | (X = any amino acid) | |
| α chain with WT signal | MKKHLTTFLVILWLYFYRGNGKNQVEQSPQSLIILEGKNCTLQCNYTVSPFS | 1358 |
| peptide, Cα | NLRWYKQDTGRGPVSLTIMTFSENTKSNGRYTATLDADTKQSSLHITASQLS | |
| (substituted) | DSASYICVVSSYKLIFGTGTRLQVFPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain with | MAKHLTTFLVILWLYFYRGNGKNQVEQSPQSLIILEGKNCTLQCNYTVSPFS | 1359 |
| alternative signal | NLRWYKQDTGRGPVSLTIMTFSENTKSNGRYTATLDADTKQSSLHITASQLS | |
| peptide, Cα | DSASYICVVSSYKLIFGTGTRLQVFPNIQNPEPAVYQLKDPRSQDSTLCLFT | |
| (substituted) | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain with | MHKHLTTFLVILWLYFYRGNGKNQVEQSPQSLIILEGKNCTLQCNYTVSPFS | 1360 |
| alternative signal | NLRWYKQDTGRGPVSLTIMTFSENTKSNGRYTATLDADTKQSSLHITASQLS | |
| peptide, Cα | DSASYICVVSSYKLIFGTGTRLQVFPNIQNPEPAVYQLKDPRSQDSTLCLFT | |
| (substituted) | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| CDR1β | SGHTA | 2351 |
| CDR2β | FQGNSA | 2352 |
| CDR3β | ASSPIQGENSPLH | 2353 |
| Vβ without signal | GVSQSPSNKVTEKGKDVELRCDPISGHTALYWYRQRLGQGLEFLIYFQGNSA | 2354 |
| peptide (SignalP) | PDKSGLPSDRFSAERTGESVSTLTIQRTQQEDSAVYLCASSPIQGENSPLHF | |
| | GNGTRLTVT | |
| Vβ without signal | GAGVSQSPSNKVTEKGKDVELRCDPISGHTALYWYRQRLGQGLEFLIYFQGN | 2355 |
| peptide (IMGT) | SAPDKSGLPSDRESAERTGESVSTLTIQRTQQEDSAVYLCASSPIQGENSPL | |
| | HFGNGTRLTVT | |
| Vβ | MXTRLLFWVAFCLLGAYHTGAGVSQSPSNKVTEKGKDVELRCDPISGHTALY | 2356 |
| | WYRQRLGQGLEFLIYFQGNSAPDKSGLPSDRFSAERTGESVSTLTIQRTQQE | 2357 |
| | DSAVYLCASSPIQGENSPLHFGNGTRLTVT | |
| | (X = any amino acid) | |
| β chain with WT signal | MGTRLLFWVAFCLLGAYHTGAGVSQSPSNKVTEKGKDVELRCDPISGHTALY | 2358 |
| peptide, Cβ | WYRQRLGQGLEFLIYFQGNSAPDKSGLPSDRFSAERTGESVSTLTIQRTQQE | |
| (substituted) | DSAVYLCASSPIQGENSPLHFGNGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLFWVAFCLLGAYHTGAGVSQSPSNKVTEKGKDVELRCDPISGHTALY | 2359 |
| signal peptide, Cβ | WYRQRLGQGLEFLIYFQGNSAPDKSGLPSDRFSAERTGESVSTLTIQRTQQE | |
| (substituted) | DSAVYLCASSPIQGENSPLHFGNGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLFWVAFCLLGAYHTGAGVSQSPSNKVTEKGKDVELRCDPISGHTALY | 2360 |
| signal peptide, Cβ | WYRQRLGQGLEFLIYFQGNSAPDKSGLPSDRFSAERTGESVSTLTIQRTQQE | |
| (substituted) | DSAVYLCASSPIQGENSPLHFGNGTRLTVTEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR036 interacts with and/or is specific for p53. In some embodiments, the peptide is from a neoantigen of p53. In some embodiments, the neoantigen has the amino acid change R248Q relative to the wild type p53 sequence. In some embodiments, TCR036 interacts with the neoantigen in the context of HLA-A*02:01, as described in International Publication No. WO 2019/067243, incorporated herein by reference in its entirety.

TABLE 6AK
Amino acid sequences of TCR037.

| | | SEQ |
|---|---|---|
| | | ID |
| Description | Sequence | NO: |
| CDR1α | SSVSVY | 1361 |
| CDR2α | YLSGSTLV | 1362 |
| CDR3α | AVSKGTGAQKLV | 1363 |
| Vα without signal | QSVTQLDSQVPVFEEAPVELRCNYSSSVSVYLFWYVQYPNQGLQLLLKYLSG | 1364 |
| peptide (SignalP) | STLVESINGFEAEFNKSQTSFHLRKPSVHISDTAEYFCAVSKGTGAQKLVFG | |
| | QGTRLTINP | |
| Vα without signal | AQSVTQLDSQVPVFEEAPVELRCNYSSSVSVYLFWYVQYPNQGLQLLLKYLS | 1365 |
| peptide (IMGT) | GSTLVESINGFEAEFNKSQTSFHLRKPSVHISDTAEYFCAVSKGTGAQKLVF | |
| | GQGTRLTINP | |
| Vα | MXLLLVPAFQVIFTLGGTRAQSVTQLDSQVPVFEEAPVELRCNYSSSVSVYL | 1366 |
| | FWYVQYPNQGLQLLLKYLSGSTLVESINGFEAEFNKSQTSFHLRKPSVHISD | |
| | TAEYFCAVSKGTGAQKLVFGQGTRLTINP | 1367 |
| | (X = any amino acid) | |
| α chain with WT signal | MLLLLVPAFQVIFTLGGTRAQSVTQLDSQVPVFEEAPVELRCNYSSSVSVYL | 1368 |
| peptide, Cα | FWYVQYPNQGLQLLLKYLSGSTLVESINGFEAEFNKSQTSFHLRKPSVHISD | |
| (substituted) | TAEYFCAVSKGTGAQKLVFGQGTRLTINPNIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain with | MALLLVPAFQVIFTLGGTRAQSVTQLDSQVPVFEEAPVELRCNYSSSVSVYL | 1369 |
| alternative signal | FWYVQYPNQGLQLLLKYLSGSTLVESINGFEAEFNKSQTSFHLRKPSVHISD | |
| peptide, Cα | TAEYFCAVSKGTGAQKLVFGQGTRLTINPNIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain with | MHLLLVPAFQVIFTLGGTRAQSVTQLDSQVPVFEEAPVELRCNYSSSVSVYL | 1370 |
| alternative signal | FWYVQYPNQGLQLLLKYLSGSTLVESINGFEAEFNKSQTSFHLRKPSVHISD | |
| peptide, Cα | TAEYFCAVSKGTGAQKLVFGQGTRLTINPNIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| CDR1β | LNHDA | 2361 |
| CDR2β | SQIVND | 2362 |
| CDR3β | ASEAF | 2363 |
| Vβ without signal | GITQSPKYLFRKEGQNVTLSCEQNLNHDAMYWYRQDPGQGLRLIYYSQIVND | 2364 |
| peptide (SignalP) | FQKGDIAEGYSVSREKKESFPLIVTSAQKNPTASYLCASEAFFGQGTRLTVV | |
| Vβ without signal | DGGITQSPKYLFRKEGQNVTLSCEQNLNHDAMYWYRQDPGQGLRLIYYSQIV | 2365 |
| peptide (IMGT) | NDFQKGDIAEGYSVSREKKESFPLIVTSAQKNPTASYLCASEAFFGQGTRLT | |
| | VV | |
| Vβ | MXNQVLCCVVLCFLGANTVDGGITQSPKYLFRKEGQNVTLSCEQNLNHDAMY | 2366 |
| | WYRQDPGQGLRLIYYSQIVNDFQKGDIAEGYSVSREKKESFPLIVTSAQKNP | 2367 |
| | TASYLCASEAFFGQGTRLTVV | |
| | (X = any amino acid) | |
| β chain with WT signal | MSNQVLCCVVLCFLGANTVDGGITQSPKYLFRKEGQNVTLSCEQNLNHDAMY | 2368 |
| peptide, Cβ | WYRQDPGQGLRLIYYSQIVNDFQKGDIAEGYSVSREKKESFPLIVTSAQKNP | |
| (substituted) | TASYLCASEAFFGQGTRLTVVEDLRNVTPPKVSLFEPSKAEIANKQKATLVC | |
| | LARGFFPDHVELSWWVNGKEVHSGVCTDPQAYKESNYSYCLSSRLRVSATFW | |
| | HNPRNHFRCQVQFHGLSEEDKWPEGSPKPVTQNISAEAWGRADCGITSASYQ | |
| | QGVLSATILYEILLGKATLYAVLVSTLVVMAMVKRKNS | |
| β chain with alternative | MANQVLCCVVLCFLGANTVDGGITQSPKYLFRKEGQNVTLSCEQNLNHDAMY | 2369 |
| signal peptide, Cβ | WYRQDPGQGLRLIYYSQIVNDFQKGDIAEGYSVSREKKESFPLIVTSAQKNP | |
| (substituted) | TASYLCASEAFFGQGTRLTVVEDLRNVTPPKVSLFEPSKAEIANKQKATLVC | |
| | LARGFFPDHVELSWWVNGKEVHSGVCTDPQAYKESNYSYCLSSRLRVSATFW | |
| | HNPRNHFRCQVQFHGLSEEDKWPEGSPKPVTQNISAEAWGRADCGITSASYQ | |
| | QGVLSATILYEILLGKATLYAVLVSTLVVMAMVKRKNS | |
| β chain with alternative | MHNQVLCCVVLCFLGANTVDGGITQSPKYLFRKEGQNVTLSCEQNLNHDAMY | 2370 |
| signal peptide, Cβ | WYRQDPGQGLRLIYYSQIVNDFQKGDIAEGYSVSREKKESFPLIVTSAQKNP | |
| (substituted) | TASYLCASEAFFGQGTRLTVVEDLRNVTPPKVSLFEPSKAEIANKQKATLVC | |
| | LARGFFPDHVELSWWVNGKEVHSGVCTDPQAYKESNYSYCLSSRLRVSATFW | |
| | HNPRNHFRCQVQFHGLSEEDKWPEGSPKPVTQNISAEAWGRADCGITSASYQ | |
| | QGVLSATILYEILLGKATLYAVLVSTLVVMAMVKRKNS | |

In some embodiments, TCR037 interacts with and/or is specific for p53. In some embodiments, the peptide is from a neoantigen of p53. In some embodiments, the neoantigen has the amino acid change R248Q relative to the wild type p53 sequence. In some embodiments, TCR037 interacts with the neoantigen in the context of HLA-A*02:01, as described in International Publication No. WO 2019/067243, incorporated herein by reference in its entirety.

TABLE 6AL
Amino acid sequences of TCR038.

| | | SEQ |
|---|---|---|
| | | ID |
| Description | Sequence | NO: |
| CDR1α | TISGTDY | 1371 |
| CDR2α | GLTSN | 1372 |
| CDR3α | ILASGAGSYQLT | 1373 |
| Vα without signal | KTTQPNSMESNEEEPVHLPCNHSTISGTDYIHWYRQLPSQGPEYVIHGLTSN | 1374 |
| peptide (SignalP) | VNNRMASLAIAEDRKSSTLILHRATLRDAAVYYCILASGAGSYQLTFGKGTK | |
| | LSVIP | |
| Vα without signal | DAKTTQPNSMESNEEEPVHLPCNHSTISGTDYIHWYRQLPSQGPEYVIHGLT | 1375 |
| peptide (IMGT) | SNVNNRMASLAIAEDRKSSTLILHRATLRDAAVYYCILASGAGSYQLTFGKG | |
| | TKLSVIP | |
| Vα | MXLVTSITVLLSLGIMGDAKTTQPNSMESNEEEPVHLPCNHSTISGTDYIHW | 1376 |
| | YRQLPSQGPEYVIHGLTSNVNNRMASLAIAEDRKSSTLILHRATLRDAAVYY | 1377 |
| | CILASGAGSYQLTFGKGTKLSVIP | |
| | (X = any amino acid) | |
| α chain with WT signal | MKLVTSITVLLSLGIMGDAKTTQPNSMESNEEEPVHLPCNHSTISGTDYIHW | 1378 |
| peptide, Cα | YRQLPSQGPEYVIHGLTSNVNNRMASLAIAEDRKSSTLILHRATLRDAAVYY | |
| (substituted) | CILASGAGSYQLTFGKGTKLSVIPNIQNPEPAVYQLKDPRSQDSTLCLFTDF | |
| | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGFNLLMTL | |
| | RLWSS | |
| α chain with | MALVTSITVLLSLGIMGDAKTTQPNSMESNEEEPVHLPCNHSTISGTDYIHW | 1379 |
| alternative signal | YRQLPSQGPEYVIHGLTSNVNNRMASLAIAEDRKSSTLILHRATLRDAAVYY | |
| peptide, Cα | CILASGAGSYQLTFGKGTKLSVIPNIQNPEPAVYQLKDPRSQDSTLCLFTDF | |
| (substituted) | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGFNLLMTL | |
| | RLWSS | |
| α chain with | MHLVTSITVLLSLGIMGDAKTTQPNSMESNEEEPVHLPCNHSTISGTDYIHW | 1380 |
| alternative signal | YRQLPSQGPEYVIHGLTSNVNNRMASLAIAEDRKSSTLILHRATLRDAAVYY | |
| peptide, Cα | CILASGAGSYQLTFGKGTKLSVIPNIQNPEPAVYQLKDPRSQDSTLCLFTDF | |
| (substituted) | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGFNLLMTL | |
| | RLWSS | |
| CDR1β | LGHD | 2371 |
| CDR2β | YNNKEL | 2372 |
| CDR3β | ASRTIGYNTEAF | 2373 |
| Vβ without signal | QTPKYLVTQMGNDKSIKCEQNLGHDTMYWYKQDSKKFLKIMFSYNNKELIIN | 2374 |
| peptide (SignalP) | ETVPNRFSPKSPDKAHLNLHINSLELGDSAVYFCASRTIGYNTEAFFGQGTR | |
| | LTVV | |
| Vβ without signal | DTAVSQTPKYLVTQMGNDKSIKCEQNLGHDTMYWYKQDSKKFLKIMFSYNNK | 2375 |
| peptide (IMGT) | ELIINETVPNRFSPKSPDKAHLNLHINSLELGDSAVYFCASRTIGYNTEAFF | |
| | GQGTRLTVV | |
| Vβ | MXCRLLCCVVFCLLQAGPLDTAVSQTPKYLVTQMGNDKSIKCEQNLGHDTMY | 2376 |
| | WYKQDSKKFLKIMFSYNNKELIINETVPNRFSPKSPDKAHLNLHINSLELGD | 2377 |
| | SAVYFCASRTIGYNTEAFFGQGTRLTVV | |
| | (X = any amino acid) | |
| β chain with WT signal | MGCRLLCCVVFCLLQAGPLDTAVSQTPKYLVTQMGNDKSIKCEQNLGHDTMY | 2378 |
| peptide, Cβ | WYKQDSKKFLKIMFSYNNKELIINETVPNRFSPKSPDKAHLNLHINSLELGD | |
| (substituted) | SAVYFCASRTIGYNTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MACRLLCCVVFCLLQAGPLDTAVSQTPKYLVTQMGNDKSIKCEQNLGHDTMY | 2379 |
| signal peptide, Cβ | WYKQDSKKFLKIMFSYNNKELIINETVPNRFSPKSPDKAHLNLHINSLELGD | |
| (substituted) | SAVYFCASRTIGYNTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MHCRLLCCVVFCLLQAGPLDTAVSQTPKYLVTQMGNDKSIKCEQNLGHDTMY | 2380 |
| signal peptide, Cβ | WYKQDSKKFLKIMFSYNNKELIINETVPNRFSPKSPDKAHLNLHINSLELGD | |
| (substituted) | SAVYFCASRTIGYNTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR038 interacts with and/or is specific for p53. In some embodiments, the peptide is from a neoantigen of p53. In some embodiments, the neoantigen has the amino acid change R248Q relative to the wild type p53 sequence. In some embodiments, TCR038 interacts with the neoantigen in the context of HLA-A*02:01, as described in International Publication No. WO 2019/067243, incorporated herein by reference in its entirety.

TABLE 6AM
Amino acid sequences of TCR039.

| | | SEQ |
|---|---|---|
| | | ID |
| Description | Sequence | NO: |
| CDR1α | VGISA | 1381 |
| CDR2α | LSSGK | 1382 |
| CDR3α | AALSYNTDKLI | 1383 |
| Vα without signal | AKNEVEQSPQNLTAQEGEFITINCSYSVGISALHWLQQHPGGGIVSLFMLSS | 1384 |
| peptide (SignalP) | GKKKHGRLIATINIQEKHSSLHITASHPRDSAVYICAALSYNTDKLIFGTGT | |
| | RLQVFP | |
| Vα without signal | KNEVEQSPQNLTAQEGEFITINCSYSVGISALHWLQQHPGGGIVSLFMLSSG | 1385 |
| peptide (IMGT) | KKKHGRLIATINIQEKHSSLHITASHPRDSAVYICAALSYNTDKLIFGTGTR | |
| | LQVFP | |
| Vα | MXKIRQFLLAILWLQLSCVSAAKNEVEQSPQNLTAQEGEFITINCSYSVGIS | 1386 |
| | ALHWLQQHPGGGIVSLFMLSSGKKKHGRLIATINIQEKHSSLHITASHPRDS | 1387 |
| | AVYICAALSYNTDKLIFGTGTRLQVFP | |
| | (X = any amino acid) | |
| α chain with WT signal | MKKIRQFLLAILWLQLSCVSAAKNEVEQSPQNLTAQEGEFITINCSYSVGIS | 1388 |
| peptide, Cα | ALHWLQQHPGGGIVSLFMLSSGKKKHGRLIATINIQEKHSSLHITASHPRDS | |
| (substituted) | AVYICAALSYNTDKLIFGTGTRLQVFPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain with | MAKIRQFLLAILWLQLSCVSAAKNEVEQSPQNLTAQEGEFITINCSYSVGIS | 1389 |
| alternative signal | ALHWLQQHPGGGIVSLFMLSSGKKKHGRLIATINIQEKHSSLHITASHPRDS | |
| peptide, Cα | AVYICAALSYNTDKLIFGTGTRLQVFPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain with | MHKIRQFLLAILWLQLSCVSAAKNEVEQSPQNLTAQEGEFITINCSYSVGIS | 1390 |
| alternative signal | ALHWLQQHPGGGIVSLFMLSSGKKKHGRLIATINIQEKHSSLHITASHPRDS | |
| peptide, Cα | AVYICAALSYNTDKLIFGTGTRLQVFPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| CDR1ß | SGHTA | 2381 |
| CDR2β | FQGTGA | 2382 |
| CDR3ß | ASSLSGLLQETQY | 2383 |
| Vβ without signal | GVSQTPSNKVTEKGKYVELRCDPISGHTALYWYRQSLGQGPEFLIYFQGTGA | 2384 |
| peptide (SignalP) | ADDSGLPNDRFFAVRPEGSVSTLKIQRTERGDSAVYLCASSLSGLLQETQYF | |
| | GPGTRLLVL | |
| Vβ without signal | GAGVSQTPSNKVTEKGKYVELRCDPISGHTALYWYRQSLGQGPEFLIYFQGT | 2385 |
| peptide (IMGT) | GAADDSGLPNDRFFAVRPEGSVSTLKIQRTERGDSAVYLCASSLSGLLQETQ | |
| | YFGPGTRLLVL | |
| Vβ | MXTRLLCWAALCLLGADHTGAGVSQTPSNKVTEKGKYVELRCDPISGHTALY | 2386 |
| | WYRQSLGQGPEFLIYFQGTGAADDSGLPNDRFFAVRPEGSVSTLKIQRTERG | 2387 |
| | DSAVYLCASSLSGLLQETQYFGPGTRLLVL | |
| | (X = any amino acid) | |
| β chain with WT signal | MGTRLLCWAALCLLGADHTGAGVSQTPSNKVTEKGKYVELRCDPISGHTALY | 2388 |
| peptide, Cβ | WYRQSLGQGPEFLIYFQGTGAADDSGLPNDRFFAVRPEGSVSTLKIQRTERG | |
| (substituted) | DSAVYLCASSLSGLLQETQYFGPGTRLLVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLCWAALCLLGADHTGAGVSQTPSNKVTEKGKYVELRCDPISGHTALY | 2389 |
| signal peptide, Cβ | WYRQSLGQGPEFLIYFQGTGAADDSGLPNDRFFAVRPEGSVSTLKIQRTERG | |
| (substituted) | DSAVYLCASSLSGLLQETQYFGPGTRLLVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLCWAALCLLGADHTGAGVSQTPSNKVTEKGKYVELRCDPISGHTALY | 2390 |
| signal peptide, Cβ | WYRQSLGQGPEFLIYFQGTGAADDSGLPNDRFFAVRPEGSVSTLKIQRTERG | |
| (substituted) | DSAVYLCASSLSGLLQETQYFGPGTRLLVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR039 interacts with and/or is specific for p53. In some embodiments, the peptide is from a neoantigen of p53. In some embodiments, the neoantigen has the amino acid change R248Q relative to the wild type p53 sequence. In some embodiments, TCR039 interacts with the neoantigen in the context of HLA-A*02:01, as described in International Publication No. WO 2019/067243, incorporated herein by reference in its entirety.

TABLE 6AN
Amino acid sequences of TCR040.

| | | SEQ |
|---|---|---|
| | | ID |
| Description | Sequence | NO: |
| CDR1α | ATGYPS | 1391 |
| CDR2α | ATKADDK | 1392 |
| CDR3α | ALSHTGSSNTGKLI | 1393 |
| Vα without signal | NSVTQMEGPVTLSEEAFLTINCTYTATGYPSLFWYVQYPGEGLQLLLKATKA | 1394 |
| peptide (SignalP) | DDKGSNKGFEATYRKETTSFHLEKGSVQVSDSAVYFCALSHTGSSNTGKLIF | |
| | GQGTRLQVKP | |
| Vα without signal | GNSVTQMEGPVTLSEEAFLTINCTYTATGYPSLFWYVQYPGEGLQLLLKATK | 1395 |
| peptide (IMGT) | ADDKGSNKGFEATYRKETTSFHLEKGSVQVSDSAVYFCALSHTGSSNTGKLI | |
| | FGQGTRLQVKP | |
| Vα | MXYSPGLVSLILLLLGRTRGNSVTQMEGPVTLSEEAFLTINCTYTATGYPSL | 1396 |
| | FWYVQYPGEGLQLLLKATKADDKGSNKGFEATYRKETTSFHLEKGSVQVSDS | 1397 |
| | AVYFCALSHTGSSNTGKLIFGQGTRLQVKP | |
| | (X = any amino acid) | |
| α chain with WT signal | MNYSPGLVSLILLLLGRTRGNSVTQMEGPVTLSEEAFLTINCTYTATGYPSL | 1398 |
| peptide, Cα | FWYVQYPGEGLQLLLKATKADDKGSNKGFEATYRKETTSFHLEKGSVQVSDS | |
| (substituted) | AVYFCALSHTGSSNTGKLIFGQGTRLQVKPNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MAYSPGLVSLILLLLGRTRGNSVTQMEGPVTLSEEAFLTINCTYTATGYPSL | 1399 |
| alternative signal | FWYVQYPGEGLQLLLKATKADDKGSNKGFEATYRKETTSFHLEKGSVQVSDS | |
| peptide, Cα | AVYFCALSHTGSSNTGKLIFGQGTRLQVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MHYSPGLVSLILLLLGRTRGNSVTQMEGPVTLSEEAFLTINCTYTATGYPSL | 1400 |
| alternative signal | FWYVQYPGEGLQLLLKATKADDKGSNKGFEATYRKETTSFHLEKGSVQVSDS | |
| peptide, Cα | AVYFCALSHTGSSNTGKLIFGQGTRLQVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| CDR1β | SGHAT | 2391 |
| CDR2ß | FQNNGV | 2392 |
| CDR3ß | ASSTGGGRHQPQH | 2393 |
| Vβ without signal | GVAQSPRYKIIEKRQSVAFWCNPISGHATLYWYQQILGQGPKLLIQFQNNGV | 2394 |
| peptide (SignalP) | VDDSQLPKDRFSAERLKGVDSTLKIQPAKLEDSAVYLCASSTGGGRHQPQHF | |
| | GDGTRLSIL | |
| Vβ without signal | EAGVAQSPRYKIIEKRQSVAFWCNPISGHATLYWYQQILGQGPKLLIQFQNN | 2395 |
| peptide (IMGT) | GVVDDSQLPKDRFSAERLKGVDSTLKIQPAKLEDSAVYLCASSTGGGRHQPQ | |
| | HFGDGTRLSIL | |
| Vβ | MXTRLLCWAALCLLGAELTEAGVAQSPRYKIIEKRQSVAFWCNPISGHATLY | 2396 |
| | WYQQILGQGPKLLIQFQNNGVVDDSQLPKDRFSAERLKGVDSTLKIQPAKLE | 2397 |
| | DSAVYLCASSTGGGRHQPQHFGDGTRLSIL | |
| | (X = any amino acid) | |
| β chain with WT signal | MGTRLLCWAALCLLGAELTEAGVAQSPRYKIIEKRQSVAFWCNPISGHATLY | 2398 |
| peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLCWAALCLLGAELTEAGVAQSPRYKIIEKRQSVAFWCNPISGHATLY | 2399 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLCWAALCLLGAELTEAGVAQSPRYKIIEKRQSVAFWCNPISGHATLY | 2400 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR040 interacts with and/or is specific for p53. In some embodiments, the peptide is from a neoantigen of p53. In some embodiments, the neoantigen has the amino acid change R248Q relative to the wild type p53 sequence. In some embodiments, TCR040 interacts with the neoantigen in the context of HLA-A*02:01, as described in International Publication No. WO 2019/067243, incorporated herein by reference in its entirety.

TABLE 6AQ
Amino acid sequences of TCR041.

| | | SEQ |
|---|---|---|
| | | ID |
| Description | Sequence | NO: |
| CDR1α | ATGYPS | 1401 |
| CDR2α | ATKADDK | 1402 |
| CDR3α | ALSQTGSSKTGKLI | 1403 |
| Vα without signal | NSVTQMEGPVTLSEEAFLTINCTYTATGYPSLFWYVQYPGEGLQLLLKATKA | 1404 |
| peptide (SignalP) | DDKGSNKGFEATYRKETTSFHLEKGSVQVSDSAVYFCALSQTGSSKTGKLIF | |
| | GQGTRLQVKP | |
| Vα without signal | GNSVTQMEGPVTLSEEAFLTINCTYTATGYPSLFWYVQYPGEGLQLLLKATK | 1405 |
| peptide (IMGT) | ADDKGSNKGFEATYRKETTSFHLEKGSVQVSDSAVYFCALSQTGSSKTGKLI | |
| | FGQGTRLQVKP | |
| Vα | MXYSPGLVSLILLLLGRTRGNSVTQMEGPVTLSEEAFLTINCTYTATGYPSL | 1406 |
| | FWYVQYPGEGLQLLLKATKADDKGSNKGFEATYRKETTSFHLEKGSVQVSDS | |
| | AVYFCALSQTGSSKTGKLIFGQGTRLQVKP | 1407 |
| | (X = any amino acid) | |
| α chain with WT signal | MNYSPGLVSLILLLLGRTRGNSVTQMEGPVTLSEEAFLTINCTYTATGYPSL | 1408 |
| peptide, Cα | FWYVQYPGEGLQLLLKATKADDKGSNKGFEATYRKETTSFHLEKGSVQVSDS | |
| (substituted) | AVYFCALSQTGSSKTGKLIFGQGTRLQVKPNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MAYSPGLVSLILLLLGRTRGNSVTQMEGPVTLSEEAFLTINCTYTATGYPSL | 1409 |
| alternative signal | FWYVQYPGEGLQLLLKATKADDKGSNKGFEATYRKETTSFHLEKGSVQVSDS | |
| peptide, Cα | AVYFCALSQTGSSKTGKLIFGQGTRLQVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MHYSPGLVSLILLLLGRTRGNSVTQMEGPVTLSEEAFLTINCTYTATGYPSL | 1410 |
| alternative signal | FWYVQYPGEGLQLLLKATKADDKGSNKGFEATYRKETTSFHLEKGSVQVSDS | |
| peptide, Cα | AVYFCALSQTGSSKTGKLIFGQGTRLQVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| CDR1ß | SGHAT | 2401 |
| CDR2ß | FQNNGV | 2402 |
| CDR3β | ASSTGGGRHQPQH | 2403 |
| Vβ without signal | GVAQSPRYKIIEKRQSVAFWCNPISGHATLYWYQQILGQGPKLLIQFQNNGV | 2404 |
| peptide (SignalP) | VDDSQLPKDRFSAERLKGVDSTLKIQPAKLEDSAVYLCASSTGGGRHQPQHF | |
| | GDGTRLSIL | |
| Vβ without signal | EAGVAQSPRYKIIEKRQSVAFWCNPISGHATLYWYQQILGQGPKLLIQFQNN | 2405 |
| peptide (IMGT) | GVVDDSQLPKDRFSAERLKGVDSTLKIQPAKLEDSAVYLCASSTGGGRHQPQ | |
| | HFGDGTRLSIL | |
| Vβ | MXTRLLCWAALCLLGAELTEAGVAQSPRYKIIEKRQSVAFWCNPISGHATLY | 2406 |
| | WYQQILGQGPKLLIQFQNNGVVDDSQLPKDRFSAERLKGVDSTLKIQPAKLE | 2407 |
| | DSAVYLCASSTGGGRHQPQHFGDGTRLSIL | |
| | (X = any amino acid) | |
| β chain with WT signal | MGTRLLCWAALCLLGAELTEAGVAQSPRYKIIEKRQSVAFWCNPISGHATLY | 2408 |
| peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLCWAALCLLGAELTEAGVAQSPRYKIIEKRQSVAFWCNPISGHATLY | 2409 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLCWAALCLLGAELTEAGVAQSPRYKIIEKRQSVAFWCNPISGHATLY | 2410 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR041 interacts with and/or is specific for p53. In some embodiments, the peptide is from a neoantigen of p53. In some embodiments, the neoantigen has the amino acid change R248Q relative to the wild type p53 sequence. In some embodiments, TCR041 interacts with the neoantigen in the context of HLA-A*02:01, as described in International Publication No. WO 2019/067243, incorporated herein by reference in its entirety.

TABLE 6AP
Amino acid sequences of TCR042.

| | | SEQ |
|---|---|---|
| | | ID |
| Description | Sequence | NO: |
| CDR1α | ATGYPS | 1411 |
| CDR2α | ATKADDK | 1412 |
| CDR3α | ALSQTGSSNTGKLI | 1413 |
| Vα without signal | NSVTQMEGPVTLSEEAFLTINCTYTATGYPSLFWYVQYPGEGLQLLLKATKA | 1414 |
| peptide (SignalP) | DDKGSNKGFEATYRKETTSFHLEKGSVQVSDSAVYFCALSQTGSSNTGKLIF | |
| | GQGTRLQVKP | |
| Vα without signal | GNSVTQMEGPVTLSEEAFLTINCTYTATGYPSLFWYVQYPGEGLQLLLKATK | 1415 |
| peptide (IMGT) | ADDKGSNKGFEATYRKETTSFHLEKGSVQVSDSAVYFCALSQTGSSNTGKLI | |
| | FGQGTRLQVKP | |
| Vα | MXYSPGLVSLILLLLGRTRGNSVTQMEGPVTLSEEAFLTINCTYTATGYPSL | 1416 |
| | FWYVQYPGEGLQLLLKATKADDKGSNKGFEATYRKETTSFHLEKGSVQVSDS | 1417 |
| | AVYFCALSQTGSSNTGKLIFGQGTRLQVKP | |
| | (X = any amino acid) | |
| α chain with WT signal | MNYSPGLVSLILLLLGRTRGNSVTQMEGPVTLSEEAFLTINCTYTATGYPSL | 1418 |
| peptide, Cα | FWYVQYPGEGLQLLLKATKADDKGSNKGFEATYRKETTSFHLEKGSVQVSDS | |
| (substituted) | AVYFCALSQTGSSNTGKLIFGQGTRLQVKPNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MAYSPGLVSLILLLLGRTRGNSVTQMEGPVTLSEEAFLTINCTYTATGYPSL | 1419 |
| alternative signal | FWYVQYPGEGLQLLLKATKADDKGSNKGFEATYRKETTSFHLEKGSVQVSDS | |
| peptide, Cα | AVYFCALSQTGSSNTGKLIFGQGTRLQVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MHYSPGLVSLILLLLGRTRGNSVTQMEGPVTLSEEAFLTINCTYTATGYPSL | 1420 |
| alternative signal | FWYVQYPGEGLQLLLKATKADDKGSNKGFEATYRKETTSFHLEKGSVQVSDS | |
| peptide, Cα | AVYFCALSQTGSSNTGKLIFGQGTRLQVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| CDR1β | SGHAT | 2411 |
| CDR2β | FQNNGV | 2412 |
| CDR3β | ASSTGGGRHQPQH | 2413 |
| Vβ without signal | GVAQSPRYKIIEKRQSVAFWCNPISGHATLYWYQQILGQGPKLLIQFQNNGV | 2414 |
| peptide (SignalP) | VDDSQLPKDRFSAERLKGVDSTLKIQPAKLEDSAVYLCASSTGGGRHQPQHF | |
| | GDGTRLSIL | |
| Vβ without signal | EAGVAQSPRYKIIEKRQSVAFWCNPISGHATLYWYQQILGQGPKLLIQFQNN | 2415 |
| peptide (IMGT) | GVVDDSQLPKDRFSAERLKGVDSTLKIQPAKLEDSAVYLCASSTGGGRHQPQ | |
| | HFGDGTRLSIL | |
| Vβ | MXTRLLCWAALCLLGAELTEAGVAQSPRYKIIEKRQSVAFWCNPISGHATLY | 2416 |
| | WYQQILGQGPKLLIQFQNNGVVDDSQLPKDRFSAERLKGVDSTLKIQPAKLE | 2417 |
| | DSAVYLCASSTGGGRHQPQHFGDGTRLSIL | |
| | (X = any amino acid) | |
| β chain with WT signal | MGTRLLCWAALCLLGAELTEAGVAQSPRYKIIEKRQSVAFWCNPISGHATLY | 2418 |
| peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLCWAALCLLGAELTEAGVAQSPRYKIIEKRQSVAFWCNPISGHATLY | 2419 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLCWAALCLLGAELTEAGVAQSPRYKIIEKRQSVAFWCNPISGHATLY | 2420 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR042 interacts with and/or is specific for p53. In some embodiments, the peptide is from a neoantigen of p53. In some embodiments, the neoantigen has the amino acid change R248Q relative to the wild type p53 sequence. In some embodiments, TCR042 interacts with the neoantigen in the context of HLA-A*02:01, as described in International Publication No. WO 2019/067243, incorporated herein by reference in its entirety.

TABLE 6AQ
Amino acid sequences of TCR043.

| | | SEQ |
|---|---|---|
| | | ID |
| Description | Sequence | NO: |
| CDR1α | ATGYPS | 1421 |
| CDR2α | ATKADDK | 1422 |
| CDR3α | ALSTTGSSNTGKLI | 1423 |
| Vα without signal | NSVTQMEGPVTLSEEAFLTINCTYTATGYPSLFWYVQYPGEGLQLLLKATKA | 1424 |
| peptide (SignalP) | DDKGSNKGFEATYRKETTSFHLEKGSVQVSDSAVYFCALSTTGSSNTGKLIF | |
| | GQGTTLQVKP | |
| Vα without signal | GNSVTQMEGPVTLSEEAFLTINCTYTATGYPSLFWYVQYPGEGLQLLLKATK | 1425 |
| peptide (IMGT) | ADDKGSNKGFEATYRKETTSFHLEKGSVQVSDSAVYFCALSTTGSSNTGKLI | |
| | FGQGTTLQVKP | |
| Vα | MXYSPGLVSLILLLLGRTRGNSVTQMEGPVTLSEEAFLTINCTYTATGYPSL | 1426 |
| | FWYVQYPGEGLQLLLKATKADDKGSNKGFEATYRKETTSFHLEKGSVQVSDS | 1427 |
| | AVYFCALSTTGSSNTGKLIFGQGTTLQVKP | |
| | (X = any amino acid) | |
| α chain with WT signal | MNYSPGLVSLILLLLGRTRGNSVTQMEGPVTLSEEAFLTINCTYTATGYPSL | 1428 |
| peptide, Cα | FWYVQYPGEGLQLLLKATKADDKGSNKGFEATYRKETTSFHLEKGSVQVSDS | |
| (substituted) | AVYFCALSTTGSSNTGKLIFGQGTTLQVKPNIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MAYSPGLVSLILLLLGRTRGNSVTQMEGPVTLSEEAFLTINCTYTATGYPSL | 1429 |
| alternative signal | FWYVQYPGEGLQLLLKATKADDKGSNKGFEATYRKETTSFHLEKGSVQVSDS | |
| peptide, Cα | AVYFCALSTTGSSNTGKLIFGQGTTLQVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MHYSPGLVSLILLLLGRTRGNSVTQMEGPVTLSEEAFLTINCTYTATGYPSL | 1430 |
| alternative signal | FWYVQYPGEGLQLLLKATKADDKGSNKGFEATYRKETTSFHLEKGSVQVSDS | |
| peptide, Cα | AVYFCALSTTGSSNTGKLIFGQGTTLQVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| CDR1β | SGHAT | 2421 |
| CDR2β | FQNNGV | 2422 |
| CDR3β | ASSTGGGRHQPQH | 2423 |
| Vβ without signal | GVAQSPRYKIIEKRQSVAFWCNPISGHATLYWYQQILGQGPKLLIQFQNNGV | 2424 |
| peptide (SignalP) | VDDSQLPKDRFSAERLKGVDSTLKIQPAKLEDSAVYLCASSTGGGRHQPQHF | |
| | GDGTRLSIL | |
| Vβ without signal | EAGVAQSPRYKIIEKRQSVAFWCNPISGHATLYWYQQILGQGPKLLIQFQNN | 2425 |
| peptide (IMGT) | GVVDDSQLPKDRFSAERLKGVDSTLKIQPAKLEDSAVYLCASSTGGGRHQPQ | |
| | HFGDGTRLSIL | |
| Vβ | MXTRLLCWAALCLLGAELTEAGVAQSPRYKIIEKRQSVAFWCNPISGHATLY | 2426 |
| | WYQQILGQGPKLLIQFQNNGVVDDSQLPKDRFSAERLKGVDSTLKIQPAKLE | 2427 |
| | DSAVYLCASSTGGGRHQPQHFGDGTRLSIL | |
| | (X = any amino acid) | |
| β chain with WT signal | MGTRLLCWAALCLLGAELTEAGVAQSPRYKIIEKRQSVAFWCNPISGHATLY | 2428 |
| peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MATRLLCWAALCLLGAELTEAGVAQSPRYKIIEKRQSVAFWCNPISGHATLY | 2429 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with alternative | MHTRLLCWAALCLLGAELTEAGVAQSPRYKIIEKRQSVAFWCNPISGHATLY | 2430 |
| signal peptide, Cβ | WYQQILGQGPKLLIQFQNNGVVDDSQLPKDRFSAERLKGVDSTLKIQPAKLE | |
| (substituted) | DSAVYLCASSTGGGRHQPQHFGDGTRLSILEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR043 interacts with and/or is specific for p53. In some embodiments, the peptide is from a neoantigen of p53. In some embodiments, the neoantigen has the amino acid change R248Q relative to the wild type p53 sequence. In some embodiments, TCR043 interacts with the neoantigen in the context of HLA-A*02:01, as described in International Publication No. WO 2019/067243, incorporated herein by reference in its entirety.

TABLE 6AR
Amino acid sequences of TCR044.

| | | SEQ |
|---|---|---|
| | | ID |
| Description | Sequence | NO: |
| CDR1α | DRGSQS | 1431 |
| CDR2α | IYSNGD | 1432 |
| CDR3α | AVSWYSTLT | 1433 |
| Vα without signal | QQKEVEQNSGPLSVPEGAIASLNCTYSDRGSQSFFWYRQYSGKSPELIMFIY | 1434 |
| peptide (SignalP) | SNGDKEDGRFTAQLNKASQYVSLLIRDSQPSDSATYLCAVSWYSTLTFGKGT | |
| | MLLVSP | |
| Vα without signal | QKEVEQNSGPLSVPEGAIASLNCTYSDRGSQSFFWYRQYSGKSPELIMFIYS | 1435 |
| peptide (IMGT) | NGDKEDGRFTAQLNKASQYVSLLIRDSQPSDSATYLCAVSWYSTLTFGKGTM | |
| | LLVSP | |
| Vα | MXSLRVLLVILWLQLSWVWSQQKEVEQNSGPLSVPEGAIASLNCTYSDRGSQ | 1436 |
| | SFFWYRQYSGKSPELIMFIYSNGDKEDGRFTAQLNKASQYVSLLIRDSQPSD | 1437 |
| | SATYLCAVSWYSTLTFGKGTMLLVSP | |
| | (X = any amino acid) | |
| α chain with WT signal | MKSLRVLLVILWLQLSWVWSQQKEVEQNSGPLSVPEGAIASLNCTYSDRGSQ | 1438 |
| peptide, Cα | SFFWYRQYSGKSPELIMFIYSNGDKEDGRFTAQLNKASQYVSLLIRDSQPSD | |
| (substituted) | SATYLCAVSWYSTLTFGKGTMLLVSPNIQNPEPAVYQLKDPRSQDSTLCLFT | |
| | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain with | MASLRVLLVILWLQLSWVWSQQKEVEQNSGPLSVPEGAIASLNCTYSDRGSQ | 1439 |
| alternative signal | SFFWYRQYSGKSPELIMFIYSNGDKEDGRFTAQLNKASQYVSLLIRDSQPSD | |
| peptide, Cα | SATYLCAVSWYSTLTFGKGTMLLVSPNIQNPEPAVYQLKDPRSQDSTLCLFT | |
| (substituted) | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| α chain with | MHSLRVLLVILWLQLSWVWSQQKEVEQNSGPLSVPEGAIASLNCTYSDRGSQ | 1440 |
| alternative signal | SFFWYRQYSGKSPELIMFIYSNGDKEDGRETAQLNKASQYVSLLIRDSQPSD | |
| peptide, Cα | SATYLCAVSWYSTLTFGKGTMLLVSPNIQNPEPAVYQLKDPRSQDSTLCLFT | |
| (substituted) | DFDSQINVPKTMESGTFITDKCVLDMKAMDSKSNGAIAWSNQTSFTCQDIFK | |
| | ETNATYPSSDVPCDATLTEKSFETDMNLNFQNLLVIVLRILLLKVAGFNLLM | |
| | TLRLWSS | |
| CDR1β | WSHSY | 2431 |
| CDR2β | SAAADI | 2432 |
| CDR3β | ASSGSRTDTQY | 2433 |
| Vβ without signal | GITQSPRYKITETGRQVTLMCHQTWSHSYMFWYRQDLGHGLRLIYYSAAADI | 2434 |
| peptide (SignalP) | TDKGEVPDGYVVSRSKTENFPLTLESATRSQTSVYFCASSGSRTDTQYFGPG | |
| | TRLTVL | |
| Vβ without signal | DAGITQSPRYKITETGRQVTLMCHQTWSHSYMFWYRQDLGHGLRLIYYSAAA | 2435 |
| peptide (IMGT) | DITDKGEVPDGYVVSRSKTENFPLTLESATRSQTSVYFCASSGSRTDTQYFG | |
| | PGTRLTVL | |
| Vβ | MXTRLFFYVALCLLWAGHRDAGITQSPRYKITETGRQVTLMCHQTWSHSYMF | 2436 |
| | WYRQDLGHGLRLIYYSAAADITDKGEVPDGYVVSRSKTENFPLTLESATRSQ | 2437 |
| | TSVYFCASSGSRTDTQYFGPGTRLTVL | |
| | (X = any amino acid) | |
| β chain with WT signal | MGTRLFFYVALCLLWAGHRDAGITQSPRYKITETGRQVTLMCHQTWSHSYMF | 2438 |
| peptide, Cβ | WYRQDLGHGLRLIYYSAAADITDKGEVPDGYVVSRSKTENFPLTLESATRSQ | |
| (substituted) | TSVYFCASSGSRTDTQYFGPGTRLTVLEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain with alternative | MATRLFFYVALCLLWAGHRDAGITQSPRYKITETGRQVTLMCHQTWSHSYMF | 2439 |
| signal peptide, Cβ | WYRQDLGHGLRLIYYSAAADITDKGEVPDGYVVSRSKTENFPLTLESATRSQ | |
| (substituted) | TSVYFCASSGSRTDTQYFGPGTRLTVLEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain with alternative | MHTRLFFYVALCLLWAGHRDAGITQSPRYKITETGRQVTLMCHQTWSHSYMF | 2440 |
| signal peptide, Cβ | WYRQDLGHGLRLIYYSAAADITDKGEVPDGYVVSRSKTENFPLTLESATRSQ | |
| (substituted) | TSVYFCASSGSRTDTQYFGPGTRLTVLEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |

In some embodiments, TCR044 interacts with and/or is specific for p53. In some embodiments, the peptide is from a neoantigen of p53. In some embodiments, the neoantigen has the amino acid change R248Q relative to the wild type p53 sequence. In some embodiments, TCR044 interacts with the neoantigen in the context of HLA-A*02:01, as described in International Publication No. WO 2019/067243, incorporated herein by reference in its entirety.

TABLE 6AS
Amino acid sequences of TCR045.

| | | SEQ |
|---|---|---|
| | | ID |
| Description | Sequence | NO: |
| CDR1α | SVFSS | 1441 |
| CDR2α | VVTGGEV | 1442 |
| CDR3α | AGEFAGNQFY | 1443 |
| Vα without signal | QLLEQSPQFLSIQEGENLTVYCNSSSVFSSLQWYRQEPGEGPVLLVTVVTGG | 1444 |
| peptide (SignalP) | EVKKLKRLTFQFGDARKDSSLHITAAQPGDTGLYLCAGEFAGNQFYFGTGTS | |
| | LTVIP | |
| Vα without signal | TQLLEQSPQFLSIQEGENLTVYCNSSSVFSSLQWYRQEPGEGPVLLVTVVTG | 1445 |
| peptide (IMGT) | GEVKKLKRLTFQFGDARKDSSLHITAAQPGDTGLYLCAGEFAGNQFYFGTGT | |
| | SLTVIP | |
| Vα | MXLKFSVSILWIQLAWVSTQLLEQSPQFLSIQEGENLTVYCNSSSVFSSLQW | 1446 |
| | YRQEPGEGPVLLVTVVTGGEVKKLKRLTFQFGDARKDSSLHITAAQPGDTGL | 1447 |
| | YLCAGEFAGNQFYFGTGTSLTVIP | |
| | (X = any amino acid) | |
| α chain with WT signal | MVLKFSVSILWIQLAWVSTQLLEQSPQFLSIQEGENLTVYCNSSSVFSSLQW | 1448 |
| peptide, Cα | YRQEPGEGPVLLVTVVTGGEVKKLKRLTFQFGDARKDSSLHITAAQPGDTGL | |
| (substituted) | YLCAGEFAGNQFYFGTGTSLTVIPNIQNPEPAVYQLKDPRSQDSTLCLFTDF | |
| | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGFNLLMTL | |
| | RLWSS | |
| α chain with | MALKFSVSILWIQLAWVSTQLLEQSPQFLSIQEGENLTVYCNSSSVFSSLQW | 1449 |
| alternative signal | YRQEPGEGPVLLVTVVTGGEVKKLKRLTFQFGDARKDSSLHITAAQPGDTGL | |
| peptide, Cα | YLCAGEFAGNQFYFGTGTSLTVIPNIQNPEPAVYQLKDPRSQDSTLCLFTDF | |
| (substituted) | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGFNLLMTL | |
| | RLWSS | |
| α chain with | MHLKFSVSILWIQLAWVSTQLLEQSPQFLSIQEGENLTVYCNSSSVFSSLQW | 1450 |
| alternative signal | YRQEPGEGPVLLVTVVTGGEVKKLKRLTFQFGDARKDSSLHITAAQPGDTGL | |
| peptide, Cα | YLCAGEFAGNQFYFGTGTSLTVIPNIQNPEPAVYQLKDPRSQDSTLCLFTDF | |
| (substituted) | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGFNLLMTL | |
| | RLWSS | |
| CDR1β | MGHRA | 2441 |
| CDR2β | YSYEKL | 2442 |
| CDR3β | ASSQVGLTYEQY | 2443 |
| Vβ without signal | EVTQTPKHLVMGMTNKKSLKCEQHMGHRAMYWYKQKAKKPPELMFVYSYEKL | 2444 |
| peptide (SignalP) | SINESVPSRFSPECPNSSLLNLHLHALQPEDSALYLCASSQVGLTYEQYFGP | |
| | GTRLTVT | |
| Vβ without signal | DTEVTQTPKHLVMGMTNKKSLKCEQHMGHRAMYWYKQKAKKPPELMFVYSYE | 2445 |
| peptide (IMGT) | KLSINESVPSRFSPECPNSSLLNLHLHALQPEDSALYLCASSQVGLTYEQYF | |
| | GPGTRLTVT | |
| Vβ | MXCRLLCCAVLCLLGAVPIDTEVTQTPKHLVMGMTNKKSLKCEQHMGHRAMY | 2446 |
| | WYKQKAKKPPELMFVYSYEKLSINESVPSRFSPECPNSSLLNLHLHALQPED | 2447 |
| | SALYLCASSQVGLTYEQYFGPGTRLTVT | |
| | (X = any amino acid) | |
| β chain with WT signal | MGCRLLCCAVLCLLGAVPIDTEVTQTPKHLVMGMTNKKSLKCEQHMGHRAMY | 2448 |
| peptide, Cβ | WYKQKAKKPPELMFVYSYEKLSINESVPSRFSPECPNSSLLNLHLHALQPED | |
| (substituted) | SALYLCASSQVGLTYEQYFGPGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MACRLLCCAVLCLLGAVPIDTEVTQTPKHLVMGMTNKKSLKCEQHMGHRAMY | 2449 |
| signal peptide, Cβ | WYKQKAKKPPELMFVYSYEKLSINESVPSRFSPECPNSSLLNLHLHALQPED | |
| (substituted) | SALYLCASSQVGLTYEQYFGPGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MHCRLLCCAVLCLLGAVPIDTEVTQTPKHLVMGMTNKKSLKCEQHMGHRAMY | 2450 |
| signal peptide, Cβ | WYKQKAKKPPELMFVYSYEKLSINESVPSRFSPECPNSSLLNLHLHALQPED | |
| (substituted) | SALYLCASSQVGLTYEQYFGPGTRLTVTEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR045 interacts with and/or is specific for p53. In some embodiments, the peptide is from a neoantigen of p53. In some embodiments, the neoantigen has the amino acid change R248Q relative to the wild type p53 sequence. In some embodiments, TCR045 interacts with the neoantigen in the context of HLA-A*02:01, as described in International Publication No. WO 2019/067243, incorporated herein by reference in its entirety.

TABLE 6AT
Amino acid sequences of TCR046.

| | | SEQ |
|---|---|---|
| | | ID |
| Description | Sequence | NO: |
| CDR1α | VSGNPY | 1451 |
| CDR2α | YITGDNLV | 1452 |
| CDR3α | AVRDNSGGSNYKLT | 1453 |
| Vα without signal | QSVAQPEDQVNVAEGNPLTVKCTYSVSGNPYLFWYVQYPNRGLQFLLKYITG | 1454 |
| peptide (SignalP) | DNLVKGSYGFEAEFNKSQTSFHLKKPSALVSDSALYFCAVRDNSGGSNYKLT | |
| | FGKGTLLTVNP | |
| Vα without signal | AQSVAQPEDQVNVAEGNPLTVKCTYSVSGNPYLFWYVQYPNRGLQFLLKYIT | 1455 |
| peptide (IMGT) | GDNLVKGSYGFEAEFNKSQTSFHLKKPSALVSDSALYFCAVRDNSGGSNYKL | |
| | TFGKGTLLTVNP | |
| Vα | MXSAPISMLAMLFTLSGLRAQSVAQPEDQVNVAEGNPLTVKCTYSVSGNPYL | 1456 |
| | FWYVQYPNRGLQFLLKYITGDNLVKGSYGFEAEFNKSQTSFHLKKPSALVSD | |
| | SALYFCAVRDNSGGSNYKLTFGKGTLLTVNP | 1457 |
| | (X = any amino acid) | |
| α chain with WT signal | MASAPISMLAMLFTLSGLRAQSVAQPEDQVNVAEGNPLTVKCTYSVSGNPYL | 1458 |
| peptide, Cα | FWYVQYPNRGLQFLLKYITGDNLVKGSYGFEAEFNKSQTSFHLKKPSALVSD | |
| (substituted) | SALYFCAVRDNSGGSNYKLTFGKGTLLTVNPNIQNPEPAVYQLKDPRSQDST | |
| | LCLFTDFDSQINVPKTMESGTFITDKCVLDMKAMDSKSNGAIAWSNQTSFTC | 1459 |
| | QDIFKETNATYPSSDVPCDATLTEKSFETDMNLNFQNLLVIVLRILLLKVAG | |
| | FNLLMTLRLWSS | |
| α chain with | MHSAPISMLAMLFTLSGLRAQSVAQPEDQVNVAEGNPLTVKCTYSVSGNPYL | 1460 |
| alternative signal | FWYVQYPNRGLQFLLKYITGDNLVKGSYGFEAEFNKSQTSFHLKKPSALVSD | |
| peptide, Cα | SALYFCAVRDNSGGSNYKLTFGKGTLLTVNPNIQNPEPAVYQLKDPRSQDST | |
| (substituted) | LCLFTDFDSQINVPKTMESGTFITDKCVLDMKAMDSKSNGAIAWSNQTSFTC | |
| | QDIFKETNATYPSSDVPCDATLTEKSFETDMNLNFQNLLVIVLRILLLKVAG | |
| | FNLLMTLRLWSS | |
| CDR1β | SGHAT | 2451 |
| CDR2β | FQNNGV | 2452 |
| CDR3β | ASSLGQGQTQY | 2453 |
| Vβ without signal | GVAQSPRYKIIEKRQSVAFWCNPISGHATLYWYQQILGQGPKLLIQFQNNGV | 2454 |
| peptide (SignalP) | VDDSQLPKDRFSAERLKGVDSTLKIQPAKLEDSAVYLCASSLGQGQTQYFGP | |
| | GTRLLVL | |
| Vβ without signal | EAGVAQSPRYKIIEKRQSVAFWCNPISGHATLYWYQQILGQGPKLLIQFQNN | 2455 |
| peptide (IMGT) | GVVDDSQLPKDRFSAERLKGVDSTLKIQPAKLEDSAVYLCASSLGQGQTQYF | |
| | GPGTRLLVL | |
| Vβ | MXTRLLCWAALCLLGAELTEAGVAQSPRYKIIEKRQSVAFWCNPISGHATLY | 2456 |
| | WYQQILGQGPKLLIQFQNNGVVDDSQLPKDRFSAERLKGVDSTLKIQPAKLE | |
| | DSAVYLCASSLGQGQTQYFGPGTRLLVL | 2457 |
| | (X = any amino acid) | |
| β chain with WT signal | MGTRLLCWAALCLLGAELTEAGVAQSPRYKIIEKRQSVAFWCNPISGHATLY | 2458 |
| peptide, Cβ | WYQQILGQGPKLLIQFQNNGVVDDSQLPKDRFSAERLKGVDSTLKIQPAKLE | |
| (substituted) | DSAVYLCASSLGQGQTQYFGPGTRLLVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MATRLLCWAALCLLGAELTEAGVAQSPRYKIIEKRQSVAFWCNPISGHATLY | 2459 |
| signal peptide, Cβ | WYQQILGQGPKLLIQFQNNGVVDDSQLPKDRFSAERLKGVDSTLKIQPAKLE | |
| (substituted) | DSAVYLCASSLGQGQTQYFGPGTRLLVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MHTRLLCWAALCLLGAELTEAGVAQSPRYKIIEKRQSVAFWCNPISGHATLY | 2460 |
| signal peptide, Cβ | WYQQILGQGPKLLIQFQNNGVVDDSQLPKDRFSAERLKGVDSTLKIQPAKLE | |
| (substituted) | DSAVYLCASSLGQGQTQYFGPGTRLLVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR046 interacts with and/or is specific for p53. In some embodiments, the peptide is from a neoantigen of p53. In some embodiments, the neoantigen has the amino acid change R248Q relative to the wild type p53 sequence. In some embodiments, TCR046 interacts with the neoantigen in the context of HLA-A*02:01, as described in International Publication No. WO 2019/067243, incorporated herein by reference in its entirety.

TABLE 6AU
Amino acid sequences of TCR047.

| | | SEQ |
|---|---|---|
| | | ID |
| Description | Sequence | NO: |
| CDR1α | SSVSVY | 1461 |
| CDR2α | YLSGSTLV | 1462 |
| CDR3α | AVRGSSGTYKYI | 1463 |
| Vα without signal | QSVTQLDSQVPVFEEAPVELRCNYSSSVSVYLFWYVQYPNQGLQLLLKYLSG | 1464 |
| peptide (SignalP) | STLVESINGFEAEFNKSQTSFHLRKPSVHISDTAEYFCAVRGSSGTYKYIFG | |
| | TGTRLKVLA | |
| Vα without signal | AQSVTQLDSQVPVFEEAPVELRCNYSSSVSVYLFWYVQYPNQGLQLLLKYLS | 1465 |
| peptide (IMGT) | GSTLVESINGFEAEFNKSQTSFHLRKPSVHISDTAEYFCAVRGSSGTYKYIF | |
| | GTGTRLKVLA | |
| Vα | MXLLLVPAFQVIFTLGGTRAQSVTQLDSQVPVFEEAPVELRCNYSSSVSVYL | 1466 |
| | FWYVQYPNQGLQLLLKYLSGSTLVESINGFEAEFNKSQTSFHLRKPSVHISD | 1467 |
| | TAEYFCAVRGSSGTYKYIFGTGTRLKVLA | |
| | (X = any amino acid) | |
| α chain with WT signal | MLLLLVPAFQVIFTLGGTRAQSVTQLDSQVPVFEEAPVELRCNYSSSVSVYL | 1468 |
| peptide, Cα | FWYVQYPNQGLQLLLKYLSGSTLVESINGFEAEFNKSQTSFHLRKPSVHISD | |
| (substituted) | TAEYFCAVRGSSGTYKYIFGTGTRLKVLANIQNPEPAVYQLKDPRSQDSTLC | |
| | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| α chain with | MALLLVPAFQVIFTLGGTRAQSVTQLDSQVPVFEEAPVELRCNYSSSVSVYL | 1469 |
| alternative signal | FWYVQYPNQGLQLLLKYLSGSTLVESINGFEAEFNKSQTSFHLRKPSVHISD | |
| peptide, Cα | TAEYFCAVRGSSGTYKYIFGTGTRLKVLANIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain with | MHLLLVPAFQVIFTLGGTRAQSVTQLDSQVPVFEEAPVELRCNYSSSVSVYL | 1470 |
| alternative signal | FWYVQYPNQGLQLLLKYLSGSTLVESINGFEAEFNKSQTSFHLRKPSVHISD | |
| peptide, Cα | TAEYFCAVRGSSGTYKYIFGTGTRLKVLANIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGFN | |
| | LLMTLRLWSS | |
| CDR1β | MDHEN | 2461 |
| CDR2β | SYDVKM | 2462 |
| CDR3β | ASKGDQNTEAF | 2463 |
| Vβ without signal | SRYLVKRTGEKVFLECVQDMDHENMFWYRQDPGLGLRLIYFSYDVKMKEKGD | 2464 |
| peptide (SignalP) | IPEGYSVSREKKERFSLILESASTNQTSMYLCASKGDQNTEAFFGQGTRLTV | |
| | V | |
| Vβ without signal | DVKVTQSSRYLVKRTGEKVFLECVQDMDHENMFWYRQDPGLGLRLIYFSYDV | 2465 |
| peptide (IMGT) | KMKEKGDIPEGYSVSREKKERFSLILESASTNQTSMYLCASKGDQNTEAFFG | |
| | QGTRLTVV | |
| Vβ | MXIRLLCRVAFCFLAVGLVDVKVTQSSRYLVKRTGEKVFLECVQDMDHENMF | 2466 |
| | WYRQDPGLGLRLIYFSYDVKMKEKGDIPEGYSVSREKKERFSLILESASTNQ | 2467 |
| | TSMYLCASKGDQNTEAFFGQGTRLTVV | |
| | (X = any amino acid) | |
| β chain with WT signal | MGIRLLCRVAFCFLAVGLVDVKVTQSSRYLVKRTGEKVFLECVQDMDHENMF | 2468 |
| peptide, Cβ | WYRQDPGLGLRLIYFSYDVKMKEKGDIPEGYSVSREKKERFSLILESASTNQ | |
| (substituted) | TSMYLCASKGDQNTEAFFGQGTRLTVVEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain with alternative | MAIRLLCRVAFCFLAVGLVDVKVTQSSRYLVKRTGEKVFLECVQDMDHENMF | 2469 |
| signal peptide, Cβ | WYRQDPGLGLRLIYFSYDVKMKEKGDIPEGYSVSREKKERFSLILESASTNQ | |
| (substituted) | TSMYLCASKGDQNTEAFFGQGTRLTVVEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHERCQVQFHGLSEEDKWPEGSPKPVTQNISAEAWGRADCGI | |
| | TSASYQQGVLSATILYEILLGKATLYAVLVSTLVVMAMVKRKNS | |
| β chain with alternative | MHIRLLCRVAFCFLAVGLVDVKVTQSSRYLVKRTGEKVFLECVQDMDHENMF | 2470 |
| signal peptide, Cβ | WYRQDPGLGLRLIYFSYDVKMKEKGDIPEGYSVSREKKERFSLILESASTNQ | |
| (substituted) | TSMYLCASKGDQNTEAFFGQGTRLTVVEDLRNVTPPKVSLFEPSKAEIANKQ | |
| | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |

In some embodiments, TCR047 interacts with and/or is specific for p53. In some embodiments, the peptide is from a neoantigen of p53. In some embodiments, the neoantigen has the amino acid change R248Q relative to the wild type p53 sequence. In some embodiments, TCR047 interacts with the neoantigen in the context of HLA-A*02:01, as described in International Publication No. WO 2019/067243, incorporated herein by reference in its entirety.

TABLE 6AV
Amino acid sequences of TCR048.

| | | SEQ |
|---|---|---|
| | | ID |
| Description | Sequence | NO: |
| CDR1α | TSGFNG | 1471 |
| CDR2α | NVLDGL | 1472 |
| CDR3α | AVRDLQTGANNLF | 1473 |
| Vα without signal | QNIDQPTEMTATEGAIVQINCTYQTSGFNGLFWYQQHAGEAPTFLSYNVLDG | 1474 |
| peptide (SignalP) | LEEKGRFSSFLSRSKGYSYLLLKELQMKDSASYLCAVRDLQTGANNLFFGTG | |
| | TRLTVIP | |
| Vα without signal | GQNIDQPTEMTATEGAIVQINCTYQTSGFNGLFWYQQHAGEAPTFLSYNVLD | 1475 |
| peptide (IMGT) | GLEEKGRFSSFLSRSKGYSYLLLKELQMKDSASYLCAVRDLQTGANNLFFGT | |
| | GTRLTVIP | |
| Vα | MXGVFLLYVSMKMGGTTGQNIDQPTEMTATEGAIVQINCTYQTSGFNGLFWY | 1476 |
| | QQHAGEAPTFLSYNVLDGLEEKGRFSSFLSRSKGYSYLLLKELQMKDSASYL | 1477 |
| | CAVRDLQTGANNLFFGTGTRLTVIP | |
| | (X = any amino acid) | |
| α chain with WT signal | MWGVFLLYVSMKMGGTTGQNIDQPTEMTATEGAIVQINCTYQTSGFNGLFWY | 1478 |
| peptide, Cα | QQHAGEAPTFLSYNVLDGLEEKGRFSSFLSRSKGYSYLLLKELQMKDSASYL | |
| (substituted) | CAVRDLQTGANNLFFGTGTRLTVIPNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGFNLLMT | |
| | LRLWSS | |
| α chain with | MAGVFLLYVSMKMGGTTGQNIDQPTEMTATEGAIVQINCTYQTSGFNGLFWY | 1479 |
| alternative signal | QQHAGEAPTFLSYNVLDGLEEKGRFSSFLSRSKGYSYLLLKELQMKDSASYL | |
| peptide, Cα | CAVRDLQTGANNLFFGTGTRLTVIPNIQNPEPAVYQLKDPRSQDSTLCLFTD | |
| (substituted) | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGFNLLMT | |
| | LRLWSS | |
| α chain with | MHGVFLLYVSMKMGGTTGQNIDQPTEMTATEGAIVQINCTYQTSGFNGLFWY | 1480 |
| alternative signal | QQHAGEAPTFLSYNVLDGLEEKGRFSSFLSRSKGYSYLLLKELQMKDSASYL | |
| peptide, Cα | CAVRDLQTGANNLFFGTGTRLTVIPNIQNPEPAVYQLKDPRSQDSTLCLFTD | |
| (substituted) | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGFNLLMT | |
| | LRLWSS | |
| CDR1β | MDHEN | 2471 |
| CDR2β | SYDVKM | 2472 |
| CDR3β | ASSLTFGTTEAF | 2473 |
| Vβ without signal | SRYLVKRTGEKVFLECVQDMDHENMFWYRQDPGLGLRLIYFSYDVKMKEKGD | 2474 |
| peptide (SignalP) | IPEGYSVSREKKERFSLILESASTNQTSMYLCASSLTFGTTEAFFGQGTRLT | |
| | VV | |
| Vβ without signal | DVKVTQSSRYLVKRTGEKVFLECVQDMDHENMFWYRQDPGLGLRLIYFSYDV | 2475 |
| peptide (IMGT) | KMKEKGDIPEGYSVSREKKERFSLILESASTNQTSMYLCASSLTFGTTEAFF | |
| | GQGTRLTVV | |
| Vβ | MXIRLLCRVAFCFLAVGLVDVKVTQSSRYLVKRTGEKVFLECVQDMDHENMF | 2476 |
| | WYRQDPGLGLRLIYFSYDVKMKEKGDIPEGYSVSREKKERFSLILESASTNQ | 2477 |
| | TSMYLCASSLTFGTTEAFFGQGTRLTVV | |
| | (X = any amino acid) | |
| β chain with WT signal | MGIRLLCRVAFCFLAVGLVDVKVTQSSRYLVKRTGEKVFLECVQDMDHENMF | 2478 |
| peptide, Cβ | WYRQDPGLGLRLIYFSYDVKMKEKGDIPEGYSVSREKKERFSLILESASTNQ | |
| (substituted) | TSMYLCASSLTFGTTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MAIRLLCRVAFCFLAVGLVDVKVTQSSRYLVKRTGEKVFLECVQDMDHENMF | 2479 |
| signal peptide, Cβ | WYRQDPGLGLRLIYFSYDVKMKEKGDIPEGYSVSREKKERFSLILESASTNQ | |
| (substituted) | TSMYLCASSLTFGTTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| β chain with alternative | MHIRLLCRVAFCFLAVGLVDVKVTQSSRYLVKRTGEKVFLECVQDMDHENMF | 2480 |
| signal peptide, Cβ | WYRQDPGLGLRLIYFSYDVKMKEKGDIPEGYSVSREKKERFSLILESASTNQ | |
| (substituted) | TSMYLCASSLTFGTTEAFFGQGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR048 interacts with and/or is specific for p53. In some embodiments, the peptide is from a neoantigen of p53. In some embodiments, the neoantigen has the amino acid change R248Q relative to the wild type p53 sequence. In some embodiments, TCR048 interacts with the neoantigen in the context of HLA-A*02:01, as described in International Publication No. WO 2019/067243, incorporated herein by reference in its entirety.

TABLE 6AW
Amino acid sequences of TCR049.

| | | SEQ |
|---|---|---|
| | | ID |
| Description | Sequence | NO: |
| CDR1α | TSGENG | 1481 |
| CDR2α | NVLDGL | 1482 |
| CDR3α | AFYYGGSQGNLI | 1483 |
| Vα without | QNIDQPTEMTATEGAIVQINCTYQTSGENGLFWYQQHAGEAPTFLSYNVLDG | 1484 |
| signal peptide | LEEKGRFSSFLSRSKGYSYLLLKELQMKDSASYLCAFYYGGSQGNLIFGKGT | |
| (SignalP) | KLSVKP | |
| Vα without | GQNIDQPTEMTATEGAIVQINCTYQTSGENGLFWYQQHAGEAPTFLSYNVLD | 1485 |
| signal peptide | GLEEKGRFSSFLSRSKGYSYLLLKELQMKDSASYLCAFYYGGSQGNLIFGKG | |
| (IMGT) | TKLSVKP | |
| Vα | MXGVFLLYVSMKMGGTTGQNIDQPTEMTATEGAIVQINCTYQTSGENGLFWY | 1486 |
| | QQHAGEAPTFLSYNVLDGLEEKGRESSFLSRSKGYSYLLLKELQMKDSASYL | 1487 |
| | CAFYYGGSQGNLIFGKGTKLSVKP | |
| | (X = any amino acid) | |
| α chain with | MWGVFLLYVSMKMGGTTGQNIDQPTEMTATEGAIVQINCTYQTSGENGLEWY | 1488 |
| WT signal | QQHAGEAPTFLSYNVLDGLEEKGRFSSFLSRSKGYSYLLLKELQMKDSASYL | |
| peptide, Cα | CAFYYGGSQGNLIFGKGTKLSVKPNIQNPEPAVYQLKDPRSQDSTLCLFTDF | |
| (substituted) | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGENLLMTL | |
| | RLWSS | |
| α chain with | MAGVFLLYVSMKMGGTTGQNIDQPTEMTATEGAIVQINCTYQTSGENGLFWY | 1489 |
| alternative | QQHAGEAPTFLSYNVLDGLEEKGRESSFLSRSKGYSYLLLKELQMKDSASYL | |
| signal peptide, | CAFYYGGSQGNLIFGKGTKLSVKPNIQNPEPAVYQLKDPRSQDSTLCLFTDE | |
| Cα | DSQINVPKTMESGTFITDKCVLDMKAMDSKSNGAIAWSNQTSFTCQDIFKET | |
| (substituted) | NATYPSSDVPCDATLTEKSFETDMNLNFQNLLVIVLRILLLKVAGENLLMTL | |
| | RLWSS | |
| α chain with | MHGVFLLYVSMKMGGTTGQNIDQPTEMTATEGAIVQINCTYQTSGFNGLFWY | 1490 |
| alternative | QQHAGEAPTFLSYNVLDGLEEKGRESSFLSRSKGYSYLLLKELQMKDSASYL | |
| signal peptide, | CAFYYGGSQGNLIFGKGTKLSVKPNIQNPEPAVYQLKDPRSQDSTLCLFTDE | |
| Cα | DSQINVPKTMESGTFITDKCVLDMKAMDSKSNGAIAWSNQTSFTCQDIFKET | |
| (substituted) | NATYPSSDVPCDATLTEKSFETDMNLNFQNLLVIVLRILLLKVAGENLLMTL | |
| | RLWSS | |
| CDR1β | SGHVS | 2481 |
| CDR2β | FQNEAQ | 2482 |
| CDR3β | ASSFGSGSTDTQY | 2483 |
| Vβ without | GVSQSPRYKVAKRGQDVALRCDPISGHVSLFWYQQALGQGPEFLTYFQNEAQ | 2484 |
| signal peptide) | LDKSGLPSDRFFAERPEGSVSTLKIQRTQQEDSAVYLCASSFGSGSTDTQYF | |
| (SignalP | GPGTRLTVL | |
| Vβ without | GAGVSQSPRYKVAKRGQDVALRCDPISGHVSLFWYQQALGQGPEFLTYFQNE | 2485 |
| signal peptide | AQLDKSGLPSDRFFAERPEGSVSTLKIQRTQQEDSAVYLCASSFGSGSTDTQ | |
| (IMGT) | YFGPGTRLTVL | |
| Vβ | MXTRLLCWVVLGFLGTDHTGAGVSQSPRYKVAKRGQDVALRCDPISGHVSLF | 2486 |
| | WYQQALGQGPEFLTYFQNEAQLDKSGLPSDRFFAERPEGSVSTLKIQRTQQE | 2487 |
| | DSAVYLCASSFGSGSTDTQYFGPGTRLTVL | |
| | (X = any amino acid) | |
| β chain with | MGTRLLCWVVLGFLGTDHTGAGVSQSPRYKVAKRGQDVALRCDPISGHVSLF | 2488 |
| WT signal | WYQQALGQGPEFLTYFQNEAQLDKSGLPSDRFFAERPEGSVSTLKIQRTQQE | |
| peptide, Cβ | DSAVYLCASSFGSGSTDTQYFGPGTRLTVLEDLRNVTPPKVSLFEPSKAEIA | |
| (substituted) | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with | MATRLLCWVVLGFLGTDHTGAGVSQSPRYKVAKRGQDVALRCDPISGHVSLF | 2489 |
| alternative | WYQQALGQGPEFLTYFQNEAQLDKSGLPSDRFFAERPEGSVSTLKIQRTQQE | |
| signal peptide, | DSAVYLCASSFGSGSTDTQYFGPGTRLTVLEDLRNVTPPKVSLFEPSKAEIA | |
| Cβ | NKQKATLVCLARGFFPDHVELSWWVNGKEVHSGVCTDPQAYKESNYSYCLSS | |
| (substituted) | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with | MHTRLLCWVVLGFLGTDHTGAGVSQSPRYKVAKRGQDVALRCDPISGHVSLF | 2490 |
| alternative | WYQQALGQGPEFLTYFQNEAQLDKSGLPSDRFFAERPEGSVSTLKIQRTQQE | |
| signal peptide, | DSAVYLCASSFGSGSTDTQYFGPGTRLTVLEDLRNVTPPKVSLFEPSKAEIA | |
| Cβ | NKQKATLVCLARGFFPDHVELSWWVNGKEVHSGVCTDPQAYKESNYSYCLSS | |
| (substituted) | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR049 interacts with and/or is specific for p53. In some embodiments, the peptide is from a neoantigen of p53. In some embodiments, the neoantigen has the amino acid change R248W relative to the wild type p53 sequence. In some embodiments, TCR049 interacts with the neoantigen in the context of HLA-A*68:01, as described in International Publication No. WO 2019/067243, incorporated herein by reference in its entirety.

TABLE 6AX
Amino acid sequences of TCR050.

| | | SEQ |
|---|---|---|
| | | ID |
| Description | Sequence | NO: |
| CDR1α | VTNERS | 1491 |
| CDR2α | LTSSGIE | 1492 |
| CDR3α | AGQNYGGSQGNLI | 1493 |
| Vα without | EDKVVQSPLSLVVHEGDTVTLNCSYEVTNFRSLLWYKQEKKAPTFLFMLTSS | 1494 |
| signal peptide | GIEKKSGRLSSILDKKELSSILNITATQTGDSAIYLCAGONYGGSQGNLIFG | |
| (SignalP) | KGTKLSVKP | |
| Vα without | EDKVVQSPLSLVVHEGDTVTLNCSYEVTNFRSLLWYKQEKKAPTFLEMLTSS | 1495 |
| signal peptide | GIEKKSGRLSSILDKKELSSILNITATQTGDSAIYLCAGQNYGGSQGNLIFG | |
| (IMGT) | KGTKLSVKP | |
| Vα | MXKCPQALLAIFWLLLSWVSSEDKVVQSPLSLVVHEGDTVTLNCSYEVTNER | 1496 |
| | SLLWYKQEKKAPTFLFMLTSSGIEKKSGRLSSILDKKELSSILNITATQTGD | 1497 |
| | SAIYLCAGQNYGGSQGNLIFGKGTKLSVKP | |
| | (X = any amino acid) | |
| α chain with | MMKCPQALLAIFWLLLSWVSSEDKVVQSPLSLVVHEGDTVTLNCSYEVTNER | 1498 |
| WT signal | SLLWYKQEKKAPTFLFMLTSSGIEKKSGRLSSILDKKELSSILNITATQTGD | |
| peptide, Cα | SAIYLCAGQNYGGSQGNLIFGKGTKLSVKPNIQNPEPAVYQLKDPRSQDSTL | |
| (substituted) | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain with | MAKCPQALLAIFWLLLSWVSSEDKVVQSPLSLVVHEGDTVTLNCSYEVTNER | 1499 |
| alternative | SLLWYKQEKKAPTFLFMLTSSGIEKKSGRLSSILDKKELSSILNITATQTGD | |
| signal peptide, | SAIYLCAGONYGGSQGNLIFGKGTKLSVKPNIONPEPAVYQLKDPRSQDSTL | |
| Cα | CLFTDFDSQINVPKTMESGTFITDKCVLDMKAMDSKSNGAIAWSNQTSFTCQ | |
| (substituted) | DIFKETNATYPSSDVPCDATLTEKSFETDMNLNFQNLLVIVLRILLLKVAGE | |
| | NLLMTLRLWSS | |
| α chain with | MHKCPQALLAIFWLLLSWVSSEDKVVQSPLSLVVHEGDTVTLNCSYEVTNER | 1500 |
| alternative | SLLWYKQEKKAPTFLFMLTSSGIEKKSGRLSSILDKKELSSILNITATQTGD | |
| signal peptide, | SAIYLCAGQNYGGSQGNLIFGKGTKLSVKPNIONPEPAVYQLKDPRSQDSTL | |
| Cα | CLFTDFDSQINVPKTMESGTFITDKCVLDMKAMDSKSNGAIAWSNQTSFTCQ | |
| (substituted) | DIFKETNATYPSSDVPCDATLTEKSFETDMNLNFQNLLVIVLRILLLKVAGF | |
| | NLLMTLRLWSS | |
| CDR1β | SNHLY | 2491 |
| CDR2β | FYNNE | 2492 |
| CDR3β | ASRDPAYEQY | 2493 |
| Vβ without | EPEVTQTPSHQVTQMGQEVILRCVPISNHLYFYWYRQILGQKVEFLVSFYNN | 2494 |
| signal peptide | EISEKSEIFDDQFSVERPDGSNFTLKIRSTKLEDSAMYFCASRDPAYEQYFG | |
| (SignalP) | PGTRLTVT | |
| Vβ without | EPEVTQTPSHQVTQMGQEVILRCVPISNHLYFYWYRQILGQKVEFLVSFYNN | 2495 |
| signal peptide | EISEKSEIFDDQFSVERPDGSNFTLKIRSTKLEDSAMYFCASRDPAYEQYFG | |
| (IMGT) | PGTRLTVT | |
| Vβ | MXTWLVCWAIFSLLKAGLTEPEVTQTPSHQVTQMGQEVILRCVPISNHLYFY | 2496 |
| | WYRQILGQKVEFLVSFYNNEISEKSEIFDDQFSVERPDGSNFTLKIRSTKLE | 2497 |
| | DSAMYFCASRDPAYEQYFGPGTRLTVT | |
| | (X = any amino acid) | |
| β chain with | MDTWLVCWAIFSLLKAGLTEPEVTQTPSHQVTOMGQEVILRCVPISNHLYFY | 2498 |
| WT signal | WYRQILGQKVEFLVSFYNNEISEKSEIFDDQFSVERPDGSNFTLKIRSTKLE | |
| peptide, Cβ | DSAMYFCASRDPAYEQYFGPGTRLTVTEDLRNVTPPKVSLFEPSKAEIANKO | |
| (substituted) | KATLVCLARGFFPDHVELSWWVNGKEVHSGVCTDPQAYKESNYSYCLSSRLR | |
| | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain with | MATWLVCWAIFSLLKAGLTEPEVTQTPSHQVTOMGQEVILRCVPISNHLYFY | 2499 |
| alternative | WYRQILGQKVEFLVSFYNNEISEKSEIFDDQFSVERPDGSNFTLKIRSTKLE | |
| signal peptide, | DSAMYFCASRDPAYEQYFGPGTRLTVTEDLRNVTPPKVSLFEPSKAEIANKQ | |
| Cβ | KATLVCLARGFFPDHVELSWWVNGKEVHSGVCTDPQAYKESNYSYCLSSRLR | |
| (substituted) | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |
| β chain with | MHTWLVCWAIFSLLKAGLTEPEVTQTPSHQVTQMGQEVILRCVPISNHLYFY | 2500 |
| alternative | WYRQILGQKVEFLVSFYNNEISEKSEIFDDQFSVERPDGSNFTLKIRSTKLE | |
| signal peptide, | DSAMYFCASRDPAYEQYFGPGTRLTVTEDLRNVTPPKVSLFEPSKAEIANKQ | |
| Cβ | KATLVCLARGFFPDHVELSWWVNGKEVHSGVCTDPQAYKESNYSYCLSSRLR | |
| (substituted) | VSATFWHNPRNHFRCQVQFHGLSEEDKWPEGSPKPVTQNISAEAWGRADCGI | |
| | TSASYQQGVLSATILYEILLGKATLYAVLVSTLVVMAMVKRKNS | |

In some embodiments, TCR050 interacts with and/or is specific for p53. In some embodiments, the peptide is from a neoantigen of p53. In some embodiments, the neoantigen has the amino acid change R248W relative to the wild type p53 sequence. In some embodiments, TCR050 interacts with the neoantigen in the context of HLA-A*02:01, as described in International Publication No. WO 2019/067243, incorporated herein by reference in its entirety.

TABLE 6AY
Amino acid sequences of TCR051.

| | | SEQ |
|---|---|---|
| | | ID |
| Description | Sequence | NO: |
| CDR1α | DRGSQS | 1501 |
| CDR2α | IYSNGD | 1502 |
| CDR3α | AVTLCGGYNKLI | 1503 |
| Vα without | QQKEVEQNSGPLSVPEGAIASLNCTYSDRGSQSFFWYRQYSGKSPELIMFIY | 1504 |
| signal peptide | SNGDKEDGRFTAQLNKASQYVSLLIRDSQPSDSATYLCAVTLCGGYNKLIFG | |
| (SignalP) | AGTRLAVHP | |
| Vα without | QKEVEQNSGPLSVPEGAIASLNCTYSDRGSQSFFWYRQYSGKSPELIMFIYS | 1505 |
| signal peptide | NGDKEDGRFTAQLNKASQYVSLLIRDSQPSDSATYLCAVTLCGGYNKLIFGA | |
| (IMGT) | GTRLAVHP | |
| Vα | MXSLRVLLVILWLQLSWVWSQQKEVEQNSGPLSVPEGAIASLNCTYSDRGSQ | 1506 |
| | SFFWYRQYSGKSPELIMFIYSNGDKEDGRFTAQLNKASQYVSLLIRDSQPSD | 1507 |
| | SATYLCAVTLCGGYNKLIFGAGTRLAVHP | |
| | (X = any amino acid) | |
| α chain with | MKSLRVLLVILWLQLSWVWSQQKEVEQNSGPLSVPEGAIASLNCTYSDRGSQ | 1508 |
| WT signal | SFFWYRQYSGKSPELIMFIYSNGDKEDGRFTAQLNKASQYVSLLIRDSQPSD | |
| peptide, Cα | SATYLCAVTLCGGYNKLIFGAGTRLAVHPNIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain with | MASLRVLLVILWLQLSWVWSQQKEVEQNSGPLSVPEGAIASLNCTYSDRGSQ | 1509 |
| alternative | SFFWYRQYSGKSPELIMFIYSNGDKEDGRFTAQLNKASQYVSLLIRDSQPSD | |
| signal peptide, | SATYLCAVTLCGGYNKLIFGAGTRLAVHPNIQNPEPAVYQLKDPRSQDSTLC | |
| Cα | LFTDFDSQINVPKTMESGTFITDKCVLDMKAMDSKSNGAIAWSNQTSFTCQD | |
| (substituted) | IFKETNATYPSSDVPCDATLTEKSFETDMNLNFQNLLVIVLRILLLKVAGEN | |
| | LLMTLRLWSS | |
| α chain with | MHSLRVLLVILWLQLSWVWSQQKEVEQNSGPLSVPEGAIASLNCTYSDRGSQ | 1510 |
| alternative | SFFWYRQYSGKSPELIMFIYSNGDKEDGRFTAQLNKASQYVSLLIRDSQPSD | |
| signal peptide, | SATYLCAVTLCGGYNKLIFGAGTRLAVHPNIQNPEPAVYQLKDPRSQDSTLC | |
| Cα | LFTDFDSQINVPKTMESGTFITDKCVLDMKAMDSKSNGAIAWSNQTSFTCQD | |
| (substituted) | IFKETNATYPSSDVPCDATLTEKSFETDMNLNFQNLLVIVLRILLLKVAGEN | |
| | LLMTLRLWSS | |
| CDR1β | LNHDA | 2501 |
| CDR2β | SQIVND | 2502 |
| CDR3β | ASSSRDYEQY | 2503 |
| Vβ without | GITQSPKYLFRKEGQNVTLSCEQNLNHDAMYWYRQDPGQGLRLIYYSQIVND | 2504 |
| signal peptide | FQKGDIVEGYSVSREKKESFPLTVTSAQKNPTAFYLCASSSRDYEQYFGPGT | |
| (SignalP) | RLTVT | |
| Vβ without | DGGITQSPKYLFRKEGQNVTLSCEQNLNHDAMYWYRQDPGQGLRLIYYSQIV | 2505 |
| signal peptide | NDFQKGDIVEGYSVSREKKESFPLTVTSAQKNPTAFYLCASSSRDYEQYFGP | |
| (IMGT) | GTRLTVT | |
| Vβ | MXNQVLCCVVLCFLGANTVDGGITQSPKYLFRKEGQNVTLSCEQNLNHDAMY | 2506 |
| | WYRQDPGQGLRLIYYSQIVNDFQKGDIVEGYSVSREKKESFPLTVTSAQKNP | 2507 |
| | TAFYLCASSSRDYEQYFGPGTRLTVT | |
| | (X = any amino acid) | |
| β chain with | MSNQVLCCVVLCFLGANTVDGGITQSPKYLFRKEGQNVTLSCEQNLNHDAMY | 2508 |
| WT signal | WYRQDPGQGLRLIYYSQIVNDFQKGDIVEGYSVSREKKESFPLTVTSAQKNP | |
| peptide, Cβ | TAFYLCASSSRDYEQYFGPGTRLTVTEDLRNVTPPKVSLFEPSKAEIANKQK | |
| (substituted) | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain with | MANQVLCCVVLCFLGANTVDGGITQSPKYLFRKEGQNVTLSCEQNLNHDAMY | 2509 |
| alternative | WYRQDPGQGLRLIYYSQIVNDFQKGDIVEGYSVSREKKESFPLTVTSAQKNP | |
| signal peptide, | TAFYLCASSSRDYEQYFGPGTRLTVTEDLRNVTPPKVSLFEPSKAEIANKQK | |
| Cβ | ATLVCLARGFFPDHVELSWWVNGKEVHSGVCTDPQAYKESNYSYCLSSRLRV | |
| (substituted) | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain with | MHNQVLCCVVLCFLGANTVDGGITQSPKYLFRKEGQNVTLSCEQNINHDAMY | 2510 |
| alternative | WYRQDPGQGLRLIYYSQIVNDFQKGDIVEGYSVSREKKESFPLTVTSAQKNP | |
| signal peptide, | TAFYLCASSSRDYEQYFGPGTRLTVTEDLRNVTPPKVSLFEPSKAEIANKQK | |
| Cβ | ATLVCLARGFFPDHVELSWWVNGKEVHSGVCTDPQAYKESNYSYCLSSRLRV | |
| (substituted) | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |

In some embodiments, TCR051 interacts with and/or is specific for p53. In some embodiments, the peptide is from a neoantigen of p53. In some embodiments, the neoantigen has the amino acid change R248W relative to the wild type p53 sequence. In some embodiments, TCR051 interacts with the neoantigen in the context of HLA-DPA1*03:01/DPB1*02:01:02, as described in International Publication No. WO 2019/067243, incorporated herein by reference in its entirety.

TABLE 6AZ
Amino acid sequences of TCR052.

| | | SEQ |
|---|---|---|
| | | ID |
| Description | Sequence | NO: |
| CDR1α | SSVSVY | 1511 |
| CDR2α | YLSGSTLV | 1512 |
| CDR3α | AVSDLVRDDKII | 1513 |
| Vα without | QSVTQLDSQVPVFEEAPVELRCNYSSSVSVYLFWYVQYPNQGLQLLLKYLSG | 1514 |
| signal peptide | STLVESINGFEAEFNKSQTSFHLRKPSVHISDTAEYFCAVSDLVRDDKIIFG | |
| (SignalP) | KGTRLHILP | |
| Vα without | AQSVTQLDSQVPVFEEAPVELRCNYSSSVSVYLFWYVQYPNQGLQLLLKYLS | 1515 |
| signal peptide | GSTLVESINGFEAEFNKSQTSFHLRKPSVHISDTAEYFCAVSDLVRDDKIIF | |
| (IMGT) | GKGTRLHILP | |
| Vα | MXLLLVPAFQVIFTLGGTRAQSVTQLDSQVPVFEEAPVELRCNYSSSVSVYL | 1516 |
| | FWYVQYPNQGLQLLLKYLSGSTLVESINGFEAEFNKSQTSFHLRKPSVHISD | 1517 |
| | TAEYFCAVSDLVRDDKIIFGKGTRLHILP | |
| | (X = any amino acid) | |
| α chain with | MLLLLVPAFQVIFTLGGTRAQSVTQLDSQVPVFEEAPVELRCNYSSSVSVYL | 1518 |
| WT signal | FWYVQYPNQGLQLLLKYLSGSTLVESINGFEAEFNKSQTSFHLRKPSVHISD | |
| peptide, Cα | TAEYFCAVSDLVRDDKIIFGKGTRLHILPNIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain with | MALLLVPAFQVIFTLGGTRAQSVTQLDSQVPVFEEAPVELRCNYSSSVSVYL | 1519 |
| alternative | FWYVQYPNQGLQLLLKYLSGSTLVESINGFEAEFNKSQTSFHLRKPSVHISD | |
| signal peptide, | TAEYFCAVSDLVRDDKIIFGKGTRLHILPNIQNPEPAVYQLKDPRSQDSTLC | |
| Cα | LFTDFDSQINVPKTMESGTFITDKCVLDMKAMDSKSNGAIAWSNQTSFTCQD | |
| (substituted) | IFKETNATYPSSDVPCDATLTEKSFETDMNLNFQNLLVIVLRILLLKVAGEN | |
| | LLMTLRLWSS | |
| α chain with | MHLLLVPAFQVIFTLGGTRAQSVTQLDSQVPVFEEAPVELRCNYSSSVSVYL | 1520 |
| alternative | FWYVQYPNQGLQLLLKYLSGSTLVESINGFEAEFNKSQTSFHLRKPSVHISD | |
| signal peptide, | TAEYFCAVSDLVRDDKIIFGKGTRLHILPNIQNPEPAVYQLKDPRSQDSTLC | |
| Cα | LFTDFDSQINVPKTMESGTFITDKCVLDMKAMDSKSNGAIAWSNQTSFTCQD | |
| (substituted) | IFKETNATYPSSDVPCDATLTEKSFETDMNLNFQNLLVIVLRILLLKVAGEN | |
| | LLMTLRLWSS | |
| CDR1β | MNHNS | 2511 |
| CDR2β | SASEGT | 2512 |
| CDR3β | ASIGGFEAF | 2513 |
| Vβ without | GVTQTPKFQVLKTGQSMTLQCAQDMNHNSMYWYRQDPGMGLRLIYYSASEGT | 2514 |
| signal peptide | TDKGEVPNGYNVSRLNKREFSLRLESAAPSQTSVYFCASIGGFEAFFGQGTR | |
| (SignalP) | LTVV | |
| Vβ without | NAGVTQTPKFQVLKTGQSMTLQCAQDMNHNSMYWYRQDPGMGLRLIYYSASE | 2515 |
| signal peptide | GTTDKGEVPNGYNVSRLNKREFSLRLESAAPSQTSVYFCASIGGFEAFFGQG | |
| (IMGT) | TRLTVV | |
| Vβ | MXIGLLCCVAFSLLWASPVNAGVTQTPKFQVLKTGQSMTLQCAQDMNHNSMY | 2516 |
| | WYRQDPGMGLRLIYYSASEGTTDKGEVPNGYNVSRLNKREFSLRLESAAPSQ | 2517 |
| | TSVYFCASIGGFEAFFGQGTRLTVV | |
| | (X = any amino acid) | |
| β chain with | MSIGLLCCVAFSLLWASPVNAGVTQTPKFQVLKTGQSMTLQCAQDMNHNSMY | 2518 |
| WT signal | WYRQDPGMGLRLIYYSASEGTTDKGEVPNGYNVSRLNKREFSLRLESAAPSQ | |
| peptide, Cβ | TSVYFCASIGGFEAFFGQGTRLTVVEDLRNVTPPKVSLFEPSKAEIANKQKA | |
| (substituted) | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| β chain with | MAIGLLCCVAFSLLWASPVNAGVTQTPKFQVLKTGQSMTLQCAQDMNHNSMY | 2519 |
| alternative | WYRQDPGMGLRLIYYSASEGTTDKGEVPNGYNVSRLNKREFSLRLESAAPSQ | |
| signal peptide, | TSVYFCASIGGFEAFFGQGTRLTVVEDLRNVTPPKVSLFEPSKAEIANKQKA | |
| Cβ | TLVCLARGFFPDHVELSWWVNGKEVHSGVCTDPQAYKESNYSYCLSSRLRVS | |
| (substituted) | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| β chain with | MHIGLLCCVAFSLLWASPVNAGVTQTPKFQVLKTGQSMTLQCAQDMNHNSMY | 2520 |
| alternative | WYRQDPGMGLRLIYYSASEGTTDKGEVPNGYNVSRLNKREFSLRLESAAPSQ | |
| signal peptide, | TSVYFCASIGGFEAFFGQGTRLTVVEDLRNVTPPKVSLFEPSKAEIANKQKA | |
| Cβ | TLVCLARGFFPDHVELSWWVNGKEVHSGVCTDPQAYKESNYSYCLSSRLRVS | |
| (substituted) | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |

In some embodiments, TCR052 interacts with and/or is specific for p53. In some embodiments, the peptide is from a neoantigen of p53. In some embodiments, the neoantigen has the amino acid change R248W relative to the wild type p53 sequence. In some embodiments, TCR052 interacts with the neoantigen in the context of HLA-A*68:01, as described in International Publication No. WO 2019/067243, incorporated herein by reference in its entirety.

TABLE 6BA
Amino acid sequences of TCR053.

| | | SEQ |
|---|---|---|
| | | ID |
| Description | Sequence | NO: |
| CDR1α | TSGFNG | 1521 |
| CDR2α | NVLDGL | 1522 |
| CDR3α | AVYPGGSQGNLI | 1523 |
| Vα without | QNIDQPTEMTATEGAIVQINCTYQTSGENGLFWYQQHAGEAPTFLSYNVLDG | 1524 |
| signal peptide | LEEKGRESSFLSRSKGYSYLLLKELQMKDSASYLCAVYPGGSQGNLIFGKGT | |
| (SignalP) | KLSVKP | |
| Vα without | GQNIDQPTEMTATEGAIVQINCTYQTSGENGLFWYQQHAGEAPTFLSYNVLD | 1525 |
| signal peptide | GLEEKGRFSSFLSRSKGYSYLLLKELQMKDSASYLCAVYPGGSQGNLIFGKG | |
| (IMGT) | TKLSVKP | |
| Vα | MXGVFLLYVSMKMGGTTGQNIDQPTEMTATEGAIVQINCTYQTSGENGLFWY | 1526 |
| | QQHAGEAPTFLSYNVLDGLEEKGRESSFLSRSKGYSYLLLKELQMKDSASYL | 1527 |
| | CAVYPGGSQGNLIFGKGTKLSVKP | |
| | (X = any amino acid) | |
| α chain with | MWGVFLLYVSMKMGGTTGQNIDQPTEMTATEGAIVQINCTYQTSGFNGLFWY | 1528 |
| WT signal | QQHAGEAPTFLSYNVLDGLEEKGRESSFLSRSKGYSYLLLKELQMKDSASYL | |
| peptide, Cα | CAVYPGGSQGNLIFGKGTKLSVKPNIQNPEPAVYQLKDPRSQDSTLCLFTDE | |
| (substituted) | DSQINVPKTMESGTFITDKCVLDMKAMDSKSNGAIAWSNQTSFTCQDIFKET | |
| | NATYPSSDVPCDATLTEKSFETDMNLNFQNLLVIVLRILLLKVAGENLLMTL | |
| | RLWSS | |
| α chain with | MAGVFLLYVSMKMGGTTGQNIDQPTEMTATEGAIVQINCTYQTSGENGLFWY | 1529 |
| alternative | QQHAGEAPTFLSYNVLDGLEEKGRFSSFLSRSKGYSYLLLKELQMKDSASYL | |
| signal peptide, | CAVYPGGSQGNLIFGKGTKLSVKPNIQNPEPAVYQLKDPRSQDSTLCLFTDE | |
| Cα | DSQINVPKTMESGTFITDKCVLDMKAMDSKSNGAIAWSNQTSFTCQDIFKET | |
| (substituted) | NATYPSSDVPCDATLTEKSFETDMNLNFQNLLVIVLRILLLKVAGENLLMTL | |
| | RLWSS | |
| α chain with | MHGVFLLYVSMKMGGTTGQNIDQPTEMTATEGAIVQINCTYQTSGENGLFWY | 1530 |
| alternative | QQHAGEAPTFLSYNVLDGLEEKGRESSFLSRSKGYSYLLLKELQMKDSASYL | |
| signal peptide, | CAVYPGGSQGNLIFGKGTKLSVKPNIQNPEPAVYQLKDPRSQDSTLCLFTDF | |
| Cα | DSQINVPKTMESGTFITDKCVLDMKAMDSKSNGAIAWSNQTSFTCQDIFKET | |
| (substituted) | NATYPSSDVPCDATLTEKSFETDMNLNFQNLLVIVLRILLLKVAGENLLMTL | |
| | RLWSS | |
| CDR1β | SGHAT | 2521 |
| CDR2β | FQNNGV | 2522 |
| CDR3β | ASSLGTGSTDTQY | 2523 |
| Vβ without | GVAQSPRYKIIEKRQSVAFWCNPISGHATLYWYQQILGQGPKLLIQFQNNGV | 2524 |
| signal peptide | VDDSQLPKDRFSAERLKGVDSTLKIQPAKLEDSAVYLCASSLGTGSTDTQYF | |
| (SignalP) | GPGTRLTVL | |
| Vβ without | EAGVAQSPRYKIIEKRQSVAFWCNPISGHATLYWYQQILGQGPKLLIQFQNN | 2525 |
| signal peptide | GVVDDSQLPKDRFSAERLKGVDSTLKIQPAKLEDSAVYLCASSLGTGSTDTQ | |
| (IMGT) | YFGPGTRLTVL | |
| Vβ | MXTRLLCWAALCLLGAELTEAGVAQSPRYKIIEKRQSVAFWCNPISGHATLY | 2526 |
| | WYQQILGQGPKLLIQFQNNGVVDDSQLPKDRFSAERLKGVDSTLKIQPAKLE | 2527 |
| | DSAVYLCASSLGTGSTDTQYFGPGTRLTVL | |
| | (X = any amino acid) | |
| β chain with | MGTRLLCWAALCLLGAELTEAGVAQSPRYKIIEKRQSVAFWCNPISGHATLY | 2528 |
| WT signal | WYQQILGQGPKLLIQFQNNGVVDDSQLPKDRFSAERLKGVDSTLKIQPAKLE | |
| peptide, Cβ | DSAVYLCASSLGTGSTDTQYFGPGTRLTVLEDLRNVTPPKVSLFEPSKAEIA | |
| (substituted) | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with | MATRLLCWAALCLLGAELTEAGVAQSPRYKIIEKRQSVAFWCNPISGHATLY | 2529 |
| alternative | WYQQILGQGPKLLIQFQNNGVVDDSQLPKDRFSAERLKGVDSTLKIQPAKLE | |
| signal peptide, | DSAVYLCASSLGTGSTDTQYFGPGTRLTVLEDLRNVTPPKVSLFEPSKAEIA | |
| Cβ | NKQKATLVCLARGFFPDHVELSWWVNGKEVHSGVCTDPQAYKESNYSYCLSS | |
| (substituted) | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with | MHTRLLCWAALCLLGAELTEAGVAQSPRYKIIEKRQSVAFWCNPISGHATLY | 2530 |
| alternative | WYQQILGQGPKLLIQFQNNGVVDDSQLPKDRFSAERLKGVDSTLKIQPAKLE | |
| signal peptide, | DSAVYLCASSLGTGSTDTQYFGPGTRLTVLEDLRNVTPPKVSLFEPSKAEIA | |
| Cβ | NKQKATLVCLARGFFPDHVELSWWVNGKEVHSGVCTDPQAYKESNYSYCLSS | |
| (substituted) | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR053 interacts with and/or is specific for p53. In some embodiments, the peptide is from a neoantigen of p53. In some embodiments, the neoantigen has the amino acid change R248W relative to the wild type p53 sequence. In some embodiments, TCR053 interacts with the neoantigen in the context of HLA-A*68:01, as described in International Publication No. WO 2019/067243, incorporated herein by reference in its entirety.

TABLE 6BB
Amino acid sequences of TCR054.

| | | SEQ |
|---|---|---|
| | | ID |
| Description | Sequence | NO: |
| CDR1α | DRGSQS | 1531 |
| CDR2α | IYSNGD | 1532 |
| CDR3α | AVTLSGGYNKLI | 1533 |
| Vα w/o signal | QQKEVEQNSGPLSVPEGAIASLNCTYSDRGSQSFFWYRQYSGKSPELIMFIY | 1534 |
| peptide | SNGDKEDGRFTAQLNKASQYVSLLIRDSQPSDSATYLCAVTLSGGYNKLIFG | |
| (SignalP) | AGTRLAVHP | |
| Vα w/o signal | QKEVEQNSGPLSVPEGAIASLNCTYSDRGSQSFFWYRQYSGKSPELIMFIYS | 1535 |
| peptide | NGDKEDGRFTAQLNKASQYVSLLIRDSQPSDSATYLCAVTLSGGYNKLIFGA | |
| (IMGT) | GTRLAVHP | |
| Vα | MXSLRVLLVILWLQLSWVWSQQKEVEQNSGPLSVPEGAIASLNCTYSDRGSQ | 1536 |
| | SFFWYRQYSGKSPELIMFIYSNGDKEDGRFTAQLNKASQYVSLLIRDSQPSD | 1537 |
| | SATYLCAVTLSGGYNKLIFGAGTRLAVHP | |
| | (X = any amino acid) | |
| α chain w/WT | MKSLRVLLVILWLQLSWVWSQQKEVEQNSGPLSVPEGAIASLNCTYSDRGSQ | 1538 |
| signal peptide, | SFFWYRQYSGKSPELIMFIYSNGDKEDGRFTAQLNKASQYVSLLIRDSQPSD | |
| Cα | SATYLCAVTLSGGYNKLIFGAGTRLAVHPNIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain w/ | MASLRVLLVILWLQLSWVWSQQKEVEQNSGPLSVPEGAIASLNCTYSDRGSQ | 1539 |
| alternative | SFFWYRQYSGKSPELIMFIYSNGDKEDGRFTAQLNKASQYVSLLIRDSQPSD | |
| signal peptide, | SATYLCAVTLSGGYNKLIFGAGTRLAVHPNIQNPEPAVYQLKDPRSQDSTLC | |
| Cα | LFTDFDSQINVPKTMESGTFITDKCVLDMKAMDSKSNGAIAWSNQTSFTCQD | |
| (substituted) | IFKETNATYPSSDVPCDATLTEKSFETDMNLNFQNLLVIVLRILLLKVAGEN | |
| | LLMTLRLWSS | |
| α chain w/ | MHSLRVLLVILWLQLSWVWSQQKEVEQNSGPLSVPEGAIASLNCTYSDRGSQ | 1540 |
| alternative | SFFWYRQYSGKSPELIMFIYSNGDKEDGRFTAQLNKASQYVSLLIRDSQPSD | |
| signal peptide, | SATYLCAVTLSGGYNKLIFGAGTRLAVHPNIQNPEPAVYQLKDPRSQDSTLC | |
| Cα | LFTDFDSQINVPKTMESGTFITDKCVLDMKAMDSKSNGAIAWSNQTSFTCQD | |
| (substituted) | IFKETNATYPSSDVPCDATLTEKSFETDMNLNFQNLLVIVLRILLLKVAGEN | |
| | LLMTLRLWSS | |
| CDR1β | LNHDA | 2531 |
| CDR2β | SQIVND | 2532 |
| CDR3β | ASSSRDYEQY | 2533 |
| Vβ w/o signal | GITQSPKYLFRKEGQNVTLSCEQNLNHDAMYWYRQDPGQGLRLIYYSQIVND | 2534 |
| peptide | FQKGDIVEGYSVSREKKESFPLTVTSAQKNPTAFYLCASSSRDYEQYFGPGT | |
| (SignalP) | RLTVT | |
| Vβ w/o signal | DGGITQSPKYLFRKEGQNVTLSCEQNLNHDAMYWYRQDPGQGLRLIYYSQIV | 2535 |
| peptide | NDFQKGDIVEGYSVSREKKESFPLTVTSAQKNPTAFYLCASSSRDYEQYFGP | |
| (IMGT) | GTRLTVT | |
| Vβ | MXNQVLCCVVLCFLGANTVDGGITQSPKYLFRKEGQNVTLSCEQNLNHDAMY | 2536 |
| | WYRQDPGQGLRLIYYSQIVNDFQKGDIVEGYSVSREKKESFPLTVTSAQKNP | 2537 |
| | TAFYLCASSSRDYEQYFGPGTRLTVT | |
| | (X = any amino acid) | |
| β chain w/WT | MSNQVLCCVVLCFLGANTVDGGITQSPKYLFRKEGQNVTLSCEQNLNHDAMY | 2538 |
| signal peptide, | WYRQDPGQGLRLIYYSQIVNDFQKGDIVEGYSVSREKKESFPLTVTSAQKNP | |
| Cβ | TAFYLCASSSRDYEQYFGPGTRLTVTEDLRNVTPPKVSLFEPSKAEIANKQK | |
| (substituted) | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain w/ | MANQVLCCVVLCFLGANTVDGGITQSPKYLFRKEGQNVTLSCEQNINHDAMY | 2539 |
| alternative | WYRQDPGQGLRLIYYSQIVNDFQKGDIVEGYSVSREKKESFPLTVTSAQKNP | |
| signal peptide, | TAFYLCASSSRDYEQYFGPGTRLTVTEDLRNVTPPKVSLFEPSKAEIANKQK | |
| Cβ | ATLVCLARGFFPDHVELSWWVNGKEVHSGVCTDPQAYKESNYSYCLSSRLRV | |
| (substituted) | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain w/ | MHNQVLCCVVLCFLGANTVDGGITQSPKYLFRKEGQNVTLSCEQNLNHDAMY | 2540 |
| alternative | WYRQDPGQGLRLIYYSQIVNDFQKGDIVEGYSVSREKKESFPLTVTSAQKNP | |
| signal peptide, | TAFYLCASSSRDYEQYFGPGTRLTVTEDLRNVTPPKVSLFEPSKAEIANKQK | |
| Cβ | ATLVCLARGFFPDHVELSWWVNGKEVHSGVCTDPQAYKESNYSYCLSSRLRV | |
| (substituted) | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |

In some embodiments, TCR054 interacts with and/or is specific for p53. In some embodiments, the peptide is from a neoantigen of p53. In some embodiments, the neoantigen has the amino acid change R248W relative to the wild type p53 sequence. In some embodiments, TCR054 interacts with the neoantigen in the context of DPA1*01:03/DBP1*02:01 as described in International Publication No. WO 2019/067243, incorporated herein by reference in its entirety.

TABLE 6BC
Amino acid sequences of TCR055.

| | | SEQ |
|---|---|---|
| | | ID |
| Description | Sequence | NO: |
| CDR1α | NSASDY | 1541 |
| CDR2α | IRSNMDK | 1542 |
| CDR3α | AEYIQGAQKLV | 1543 |
| Vα without | ESVGLHLPTLSVQEGDNSIINCAYSNSASDYFIWYKQESGKGPQFIIDIRSN | 1544 |
| signal peptide | MDKRQGQRVTVLLNKTVKHLSLQIAATQPGDSAVYFCAEYIQGAQKLVFGQG | |
| (SignalP) | TRLTINP | |
| Vα without | GESVGLHLPTLSVQEGDNSIINCAYSNSASDYFIWYKQESGKGPQFIIDIRS | 1545 |
| signal peptide | NMDKRQGQRVTVLLNKTVKHLSLQIAATQPGDSAVYFCAEYIQGAQKLVFGQ | |
| (IMGT) | GTRLTINP | |
| Vα | MXGIRALFMYLWLQLDWVSRGESVGLHLPTLSVQEGDNSIINCAYSNSASDY | 1546 |
| | FIWYKQESGKGPQFIIDIRSNMDKRQGQRVTVLLNKTVKHLSLQIAATQPGD | 1547 |
| | SAVYFCAEYIQGAQKLVFGQGTRLTINP | |
| | (X = any amino acid) | |
| α chain with | MAGIRALFMYLWLQLDWVSRGESVGLHLPTLSVQEGDNSIINCAYSNSASDY | 1548 |
| WT signal | FIWYKQESGKGPQFIIDIRSNMDKRQGQRVTVLLNKTVKHLSLQIAATQPGD | |
| peptide, Cα | SAVYFCAEYIQGAQKLVFGQGTRLTINPNIQNPEPAVYQLKDPRSQDSTLCL | |
| (substituted) | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| α chain with | MAGIRALFMYLWLQLDWVSRGESVGLHLPTLSVQEGDNSIINCAYSNSASDY | 1549 |
| alternative | FIWYKQESGKGPQFIIDIRSNMDKRQGQRVTVLLNKTVKHLSLQIAATQPGD | |
| signal peptide, | SAVYFCAEYIQGAQKLVFGQGTRLTINPNIQNPEPAVYQLKDPRSQDSTLCL | |
| Cα | FTDFDSQINVPKTMESGTFITDKCVLDMKAMDSKSNGAIAWSNQTSFTCQDI | |
| (substituted) | FKETNATYPSSDVPCDATLTEKSFETDMNLNFQNLLVIVLRILLLKVAGENL | |
| | LMTLRLWSS | |
| α chain with | MHGIRALFMYLWLQLDWVSRGESVGLHLPTLSVQEGDNSIINCAYSNSASDY | 1550 |
| alternative | FIWYKQESGKGPQFIIDIRSNMDKRQGQRVTVLLNKTVKHLSLQIAATQPGD | |
| signal peptide, | SAVYFCAEYIQGAQKLVFGQGTRLTINPNIQNPEPAVYQLKDPRSQDSTLCL | |
| Cα | FTDFDSQINVPKTMESGTFITDKCVLDMKAMDSKSNGAIAWSNQTSFTCQDI | |
| (substituted) | FKETNATYPSSDVPCDATLTEKSFETDMNLNFQNLLVIVLRILLLKVAGENL | |
| | LMTLRLWSS | |
| CDR1β | LGHNA | 2541 |
| CDR2β | YSLEER | 2542 |
| CDR3β | ASSQEDNEQF | 2543 |
| Vβ without | ELVPMETGVTQTPRHLVMGMTNKKSLKCEQHLGHNAMYWYKQSAKKPLELMF | 2544 |
| signal peptide | VYSLEERVENNSVPSRFSPECPNSSHLFLHLHTLQPEDSALYLCASSQEDNE | |
| (SignalP) | QFFGPGTRLTVL | |
| Vβ without | ETGVTQTPRHLVMGMTNKKSLKCEQHLGHNAMYWYKQSAKKPLELMFVYSLE | 2545 |
| signal peptide | ERVENNSVPSRESPECPNSSHLFLHLHTLQPEDSALYLCASSQEDNEQFFGP | |
| (IMGT) | GTRLTVL | |
| Vβ | MXCRLLCCAVLCLLGAGELVPMETGVTQTPRHLVMGMTNKKSLKCEQHLGHN | 2546 |
| | AMYWYKQSAKKPLELMFVYSLEERVENNSVPSRFSPECPNSSHLFLHLHTLQ | |
| | PEDSALYLCASSQEDNEQFFGPGTRLTVL | 2547 |
| | (X = any amino acid) | |
| β chain with | MGCRLLCCAVLCLLGAGELVPMETGVTQTPRHLVMGMTNKKSLKCEQHLGHN | 2548 |
| WT signal | AMYWYKQSAKKPLELMFVYSLEERVENNSVPSRESPECPNSSHLFLHLHTLQ | |
| peptide, Cβ | PEDSALYLCASSQEDNEQFFGPGTRLTVLEDLRNVTPPKVSLFEPSKAEIAN | |
| (substituted) | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| chain with | MACRLLCCAVLCLLGAGELVPMETGVTQTPRHLVMGMTNKKSLKCEQHLGHN | 2549 |
| alternative | AMYWYKQSAKKPLELMFVYSLEERVENNSVPSRESPECPNSSHLFLHLHTLQ | |
| signal peptide, | PEDSALYLCASSQEDNEQFFGPGTRLTVLEDLRNVTPPKVSLFEPSKAEIAN | |
| Cβ | KQKATLVCLARGFFPDHVELSWWVNGKEVHSGVCTDPQAYKESNYSYCLSSR | |
| (substituted) | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain with | MHCRLLCCAVLCLLGAGELVPMETGVTQTPRHLVMGMTNKKSLKCEQHLGHN | 2550 |
| alternative | AMYWYKQSAKKPLELMFVYSLEERVENNSVPSRFSPECPNSSHLFLHLHTLQ | |
| signal peptide, | PEDSALYLCASSQEDNEQFFGPGTRLTVLEDLRNVTPPKVSLFEPSKAEIAN | |
| Cβ | KQKATLVCLARGFFPDHVELSWWVNGKEVHSGVCTDPQAYKESNYSYCLSSR | |
| (substituted) | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR055 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR055 interacts with the neoantigen in the context of HLA-C*01:02, as described in International Publication No. WO 2021/163477, incorporated herein by reference in its entirety.

TABLE 6BD
Amino acid sequences of TCR056.

| | | SEQ |
|---|---|---|
| | | ID |
| Description | Sequence | NO: |
| CDR1α | DRGSQS | 1551 |
| CDR2α | IYSNGD | 1552 |
| CDR3α | AVNPPVKTSYDKVI | 1553 |
| Vα without | QQKEVEQNSGPLSVPEGAIASLNCTYSDRGSQSFFWYRQYSGKSPELIMFIY | 1554 |
| signal peptide | SNGDKEDGRFTAQLNKASQYVSLLIRDSQPSDSATYLCAVNPPVKTSYDKVI | |
| (SignalP) | FGPGTSLSVIP | |
| Vα without | QKEVEQNSGPLSVPEGAIASLNCTYSDRGSQSFFWYRQYSGKSPELIMFIYS | 1555 |
| signal peptide | NGDKEDGRFTAQLNKASQYVSLLIRDSQPSDSATYLCAVNPPVKTSYDKVIF | |
| (IMGT) | GPGTSLSVIP | |
| Vα | MXSLRVLLVILWLQLSWVWSQQKEVEQNSGPLSVPEGAIASLNCTYSDRGSQ | 1556 |
| | SFFWYRQYSGKSPELIMFIYSNGDKEDGRFTAQLNKASQYVSLLIRDSQPSD | 1557 |
| | SATYLCAVNPPVKTSYDKVIFGPGTSLSVIP | |
| | (X = any amino acid) | |
| α chain with | MKSLRVLLVILWLQLSWVWSQQKEVEQNSGPLSVPEGAIASLNCTYSDRGSQ | 1558 |
| WT signal | SFFWYRQYSGKSPELIMFIYSNGDKEDGRFTAQLNKASQYVSLLIRDSQPSD | |
| peptide, Cα | SATYLCAVNPPVKTSYDKVIFGPGTSLSVIPNIQNPEPAVYQLKDPRSQDST | |
| (substituted) | LCLFTDFDSQINVPKTMESGTFITDKCVLDMKAMDSKSNGAIAWSNQTSFTC | |
| | QDIFKETNATYPSSDVPCDATLTEKSFETDMNLNFQNLLVIVLRILLLKVAG | |
| | FNLLMTLRLWSS | |
| α chain with | MASLRVLLVILWLQLSWVWSQQKEVEQNSGPLSVPEGAIASLNCTYSDRGSQ | 1559 |
| alternative | SFFWYRQYSGKSPELIMFIYSNGDKEDGRFTAQLNKASQYVSLLIRDSQPSD | |
| signal peptide, | SATYLCAVNPPVKTSYDKVIFGPGTSLSVIPNIQNPEPAVYQLKDPRSQDST | |
| Cα | LCLFTDFDSQINVPKTMESGTFITDKCVLDMKAMDSKSNGAIAWSNQTSFTC | |
| (substituted) | QDIFKETNATYPSSDVPCDATLTEKSFETDMNLNFQNLLVIVLRILLLKVAG | |
| | FNLLMTLRLWSS | |
| α chain with | MHSLRVLLVILWLQLSWVWSQQKEVEQNSGPLSVPEGAIASLNCTYSDRGSQ | 1560 |
| alternative | SFFWYRQYSGKSPELIMFIYSNGDKEDGRFTAQLNKASQYVSLLIRDSQPSD | |
| signal peptide, | SATYLCAVNPPVKTSYDKVIFGPGTSLSVIPNIQNPEPAVYQLKDPRSQDST | |
| Cα | LCLFTDFDSQINVPKTMESGTFITDKCVLDMKAMDSKSNGAIAWSNQTSFTC | |
| (substituted) | QDIFKETNATYPSSDVPCDATLTEKSFETDMNLNFQNLLVIVLRILLLKVAG | |
| | FNLLMTLRLWSS | |
| CDR1β | SGHVS | 2551 |
| CDR2β | FNYEA | 2552 |
| CDR3β | ASSHREPHTGELF | 2553 |
| Vβ without | GVSQSPRYKVTKRGQDVALRCDPISGHVSLYWYRQALGQGPEFLTYFNYEAQ | 2554 |
| signal peptide | QDKSGLPNDRFSAERPEGSISTLTIQRTEQRDSAMYRCASSHREPHTGELFF | |
| (SignalP) | GEGSRLTVL | |
| Vβ without | GAGVSQSPRYKVTKRGQDVALRCDPISGHVSLYWYRQALGQGPEFLTYFNYE | 2555 |
| signal peptide | AQQDKSGLPNDRESAERPEGSISTLTIQRTEQRDSAMYRCASSHREPHTGEL | |
| (IMGT) | FFGEGSRLTVL | |
| Vβ | MXTSLLCWVVLGFLGTDHTGAGVSQSPRYKVTKRGQDVALRCDPISGHVSLY | 2556 |
| | WYRQALGQGPEFLTYFNYEAQQDKSGLPNDRFSAERPEGSISTLTIQRTEQR | 2557 |
| | DSAMYRCASSHREPHTGELFFGEGSRLTVL | |
| | (X = any amino acid) | |
| β chain with | MGTSLLCWVVLGFLGTDHTGAGVSQSPRYKVTKRGQDVALRCDPISGHVSLY | 2558 |
| WT signal | WYRQALGQGPEFLTYFNYEAQQDKSGLPNDRFSAERPEGSISTLTIQRTEQR | |
| peptide, Cβ | DSAMYRCASSHREPHTGELFFGEGSRLTVLEDLRNVTPPKVSLFEPSKAEIA | |
| (substituted) | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with | MATSLLCWVVLGFLGTDHTGAGVSQSPRYKVTKRGQDVALRCDPISGHVSLY | 2559 |
| alternative | WYRQALGQGPEFLTYFNYEAQQDKSGLPNDRFSAERPEGSISTLTIQRTEQR | |
| signal peptide, | DSAMYRCASSHREPHTGELFFGEGSRLTVLEDLRNVTPPKVSLFEPSKAEIA | |
| Cβ | NKQKATLVCLARGFFPDHVELSWWVNGKEVHSGVCTDPQAYKESNYSYCLSS | |
| (substituted) | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with | MHTSLLCWVVLGFLGTDHTGAGVSQSPRYKVTKRGQDVALRCDPISGHVSLY | 2560 |
| alternative | WYRQALGQGPEFLTYFNYEAQQDKSGLPNDRFSAERPEGSISTLTIQRTEQR | |
| signal peptide, | DSAMYRCASSHREPHTGELFFGEGSRLTVLEDLRNVTPPKVSLFEPSKAEIA | |
| Cβ | NKQKATLVCLARGFFPDHVELSWWVNGKEVHSGVCTDPQAYKESNYSYCLSS | |
| (substituted) | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR056 interacts with and/or is specific for p53. In some embodiments, the peptide is from a neoantigen of p53. In some embodiments, the neoantigen has the amino acid change R248W relative to the wild type p53 sequence. In some embodiments, TCR056 interacts with the neoantigen in the context of HLA-A*02:01, as described in International Publication No. WO 2019/067243, incorporated herein by reference in its entirety.

TABLE 6BE
Amino acid sequences of TCR057.

| | | SEQ |
|---|---|---|
| | | ID |
| Description | Sequence | NO: |
| CDR1α | TSGENG | 1561 |
| CDR2α | NVLDGL | 1562 |
| CDR3α | AVYTGGFKTI | 1563 |
| Vα without | QNIDQPTEMTATEGAIVQINCTYQTSGENGLFWYQQHAGEAPTFLSYNVLDG | 1564 |
| signal peptide | LEEKGRESSFLSRSKGYSYLLLKELQMKDSASYLCAVYTGGFKTIFGAGTRL | |
| (SignalP) | FVKA | |
| Vα without | GQNIDQPTEMTATEGAIVQINCTYQTSGENGLFWYQQHAGEAPTFLSYNVLD | 1565 |
| signal peptide | GLEEKGRFSSFLSRSKGYSYLLLKELQMKDSASYLCAVYTGGFKTIFGAGTR | |
| (IMGT) | LFVKA | |
| Vα | MXGVFLLYVSMKMGGTTGQNIDQPTEMTATEGAIVQINCTYQTSGENGLFWY | 1566 |
| | QQHAGEAPTFLSYNVLDGLEEKGRESSFLSRSKGYSYLLLKELQMKDSASYL | 1567 |
| | CAVYTGGFKTIFGAGTRLFVKA | |
| | (X = any amino acid) | |
| α chain with | MWGVFLLYVSMKMGGTTGQNIDQPTEMTATEGAIVQINCTYQTSGENGLFWY | 1568 |
| WT signal | QQHAGEAPTFLSYNVLDGLEEKGRESSFLSRSKGYSYLLLKELQMKDSASYL | |
| peptide, Cα | CAVYTGGFKTIFGAGTRLFVKANIQNPEPAVYQLKDPRSQDSTLCLFTDEDS | |
| (substituted) | QINVPKTMESGTFITDKCVLDMKAMDSKSNGAIAWSNQTSFTCQDIFKETNA | |
| | TYPSSDVPCDATLTEKSFETDMNLNFQNLLVIVLRILLLKVAGENLLMTLRL | |
| | WSS | |
| α chain with | MAGVFLLYVSMKMGGTTGQNIDQPTEMTATEGAIVQINCTYQTSGFNGLFWY | 1569 |
| alternative | QQHAGEAPTFLSYNVLDGLEEKGRFSSFLSRSKGYSYLLLKELQMKDSASYL | |
| signal peptide, | CAVYTGGFKTIFGAGTRLFVKANIQNPEPAVYQLKDPRSQDSTLCLFTDFDS | |
| Cα | QINVPKTMESGTFITDKCVLDMKAMDSKSNGAIAWSNQTSFTCQDIFKETNA | |
| (substituted) | TYPSSDVPCDATLTEKSFETDMNLNFQNLLVIVLRILLLKVAGENLLMTLRL | |
| | WSS | |
| α chain with | MHGVFLLYVSMKMGGTTGQNIDQPTEMTATEGAIVQINCTYQTSGENGLFWY | 1570 |
| alternative | QQHAGEAPTFLSYNVLDGLEEKGRESSFLSRSKGYSYLLLKELQMKDSASYL | |
| signal peptide, | CAVYTGGFKTIFGAGTRLFVKANIQNPEPAVYQLKDPRSQDSTLCLFTDEDS | |
| Cα | QINVPKTMESGTFITDKCVLDMKAMDSKSNGAIAWSNQTSFTCQDIFKETNA | |
| (substituted) | TYPSSDVPCDATLTEKSFETDMNLNFQNLLVIVLRILLLKVAGENLLMTLRL | |
| | WSS | |
| CDR1β | SGHAT | 2561 |
| CDR2β | FQNNGV | 2562 |
| CDR3β | ASNLGGGSTDTQY | 2563 |
| Vβ without | GVAQSPRYKIIEKRQSVAFWCNPISGHATLYWYQQILGQGPKLLIQFQNNGV | 2564 |
| signal peptide | VDDSQLPKDRFSAERLKGVDSTLKIQPAKLEDSAVYLCASNLGGGSTDTQYF | |
| (SignalP) | GPGTRLTVL | |
| Vβ without | EAGVAQSPRYKIIEKRQSVAFWCNPISGHATLYWYQQILGQGPKLLIQFQNN | 2565 |
| signal peptide | GVVDDSQLPKDRFSAERLKGVDSTLKIQPAKLEDSAVYLCASNLGGGSTDTQ | |
| (IMGT) | YFGPGTRLTVL | |
| Vβ | MXTRLLCWAALCLLGAELTEAGVAQSPRYKIIEKRQSVAFWCNPISGHATLY | 2566 |
| | WYQQILGQGPKLLIQFQNNGVVDDSQLPKDRFSAERLKGVDSTLKIQPAKLE | 2567 |
| | DSAVYLCASNLGGGSTDTQYFGPGTRLTVL | |
| | (X = any amino acid) | |
| β chain with | MGTRLLCWAALCLLGAELTEAGVAQSPRYKIIEKRQSVAFWCNPISGHATLY | 2568 |
| WT signal | WYQQILGQGPKLLIQFQNNGVVDDSQLPKDRFSAERLKGVDSTLKIQPAKLE | |
| peptide, Cβ | DSAVYLCASNLGGGSTDTQYFGPGTRLTVLEDLRNVTPPKVSLFEPSKAEIA | |
| (substituted) | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with | MATRLLCWAALCLLGAELTEAGVAQSPRYKIIEKRQSVAFWCNPISGHATLY | 2569 |
| alternative | WYQQILGQGPKLLIQFQNNGVVDDSQLPKDRFSAERLKGVDSTLKIQPAKLE | |
| signal peptide, | DSAVYLCASNLGGGSTDTQYFGPGTRLTVLEDLRNVTPPKVSLFEPSKAEIA | |
| Cβ | NKQKATLVCLARGFFPDHVELSWWVNGKEVHSGVCTDPQAYKESNYSYCLSS | |
| (substituted) | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| β chain with | MHTRLLCWAALCLLGAELTEAGVAQSPRYKIIEKRQSVAFWCNPISGHATLY | 2570 |
| alternative | WYQQILGQGPKLLIQFQNNGVVDDSQLPKDRFSAERLKGVDSTLKIQPAKLE | |
| signal peptide, | DSAVYLCASNLGGGSTDTQYFGPGTRLTVLEDLRNVTPPKVSLFEPSKAEIA | |
| Cβ | NKQKATLVCLARGFFPDHVELSWWVNGKEVHSGVCTDPQAYKESNYSYCLSS | |
| (substituted) | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR057 interacts with and/or is specific for p53. In some embodiments, the peptide is from a neoantigen of p53. In some embodiments, the neoantigen has the amino acid change R248W relative to the wild type p53 sequence. In some embodiments, TCR057 interacts with the neoantigen in the context of HLA-A*68:01, as described in International Publication No. WO 2019/067243, incorporated herein by reference in its entirety.

TABLE 6BF
Amino acid sequences of TCR058.

| | | SEQ |
|---|---|---|
| | | ID |
| Description | Sequence | NO: |
| CDR1α | NSASQS | 1571 |
| CDR2α | VYSSGN | 1572 |
| CDR3α | AVNPKYTGGFKTI | 1573 |
| Vα without | QRKEVEQDPGPFNVPEGATVAFNCTYSNSASQSFFWYRQDCRKEPKLLMSVY | 1574 |
| signal peptide | SSGNEDGRFTAHLNRASQYISLLIRDSKLSDSATYLCAVNPKYTGGFKTIFG | |
| (SignalP) | AGTRLFVKA | |
| Vα without | RKEVEQDPGPFNVPEGATVAFNCTYSNSASQSFFWYRQDCRKEPKLLMSVYS | 1575 |
| signal peptide | SGNEDGRFTAHLNRASQYISLLIRDSKLSDSATYLCAVNPKYTGGFKTIFGA | |
| (IMGT) | GTRLFVKA | |
| Vα | MXSLRVLLVILWLQLSWVWSQRKEVEQDPGPFNVPEGATVAFNCTYSNSASQ | 1576 |
| | SFFWYRQDCRKEPKLLMSVYSSGNEDGRFTAHLNRASQYISLLIRDSKLSDS | 1577 |
| | ATYLCAVNPKYTGGFKTIFGAGTRLFVKA | |
| | (X = any amino acid) | |
| α chain with | MISLRVLLVILWLQLSWVWSQRKEVEQDPGPFNVPEGATVAFNCTYSNSASQ | 1578 |
| WT signal | SFFWYRQDCRKEPKLLMSVYSSGNEDGRFTAHLNRASQYISLLIRDSKLSDS | |
| peptide, Cα | ATYLCAVNPKYTGGFKTIFGAGTRLFVKANIQNPEPAVYQLKDPRSQDSTLC | |
| (substituted) | LFTDFDSQINVPKTMESGTFITDKCVLDMKAMDSKSNGAIAWSNQTSFTCQD | |
| | IFKETNATYPSSDVPCDATLTEKSFETDMNLNFQNLLVIVLRILLLKVAGEN | |
| | LLMTLRLWSS | |
| α chain with | MASLRVLLVILWLQLSWVWSQRKEVEQDPGPFNVPEGATVAFNCTYSNSASQ | 1579 |
| alternative | SFFWYRQDCRKEPKLLMSVYSSGNEDGRFTAHLNRASQYISLLIRDSKLSDS | |
| signal peptide, | ATYLCAVNPKYTGGFKTIFGAGTRLFVKANIQNPEPAVYQLKDPRSQDSTLC | |
| Cα | LFTDFDSQINVPKTMESGTFITDKCVLDMKAMDSKSNGAIAWSNQTSFTCQD | |
| (substituted) | IFKETNATYPSSDVPCDATLTEKSFETDMNLNFQNLLVIVLRILLLKVAGEN | |
| | LLMTLRLWSS | |
| α chain with | MHSLRVLLVILWLQLSWVWSQRKEVEQDPGPFNVPEGATVAFNCTYSNSASQ | 1580 |
| alternative | SFFWYRQDCRKEPKLLMSVYSSGNEDGRFTAHLNRASQYISLLIRDSKLSDS | |
| signal peptide, | ATYLCAVNPKYTGGFKTIFGAGTRLFVKANIQNPEPAVYQLKDPRSQDSTLC | |
| Cα | LFTDFDSQINVPKTMESGTFITDKCVLDMKAMDSKSNGAIAWSNQTSFTCQD | |
| (substituted) | IFKETNATYPSSDVPCDATLTEKSFETDMNLNFQNLLVIVLRILLLKVAGEN | |
| | LLMTLRLWSS | |
| CDR1β | LGHNA | 2571 |
| CDR2β | YSLEER | 2572 |
| CDR3β | ASSQDVTSEWVDTIY | 2573 |
| Vβ without | ELVPMETGVTQTPRHLVMGMTNKKSLKCEQHLGHNAMYWYKQSAKKPLELMF | 2574 |
| signal peptide | VYSLEERVENNSVPSRFSPECPNSSHLFLHLHTLQPEDSALYLCASSQDVTS | |
| (SignalP) | EWVDTIYFGEGSWLTVV | |
| Vβ without | ETGVTQTPRHLVMGMTNKKSLKCEQHLGHNAMYWYKQSAKKPLELMFVYSLE | 2575 |
| signal peptide | ERVENNSVPSRFSPECPNSSHLFLHLHTLQPEDSALYLCASSQDVTSEWVDT | |
| (IMGT) | IYFGEGSWLTVV | |
| Vβ | MXCRLLCCAVLCLLGAGELVPMETGVTQTPRHLVMGMTNKKSLKCEQHLGHN | 2576 |
| | AMYWYKQSAKKPLELMFVYSLEERVENNSVPSRFSPECPNSSHLFLHLHTLQ | 2577 |
| | PEDSALYLCASSQDVTSEWVDTIYFGEGSWLTVV | |
| | (X = any amino acid) | |
| β chain with | MGCRLLCCAVLCLLGAGELVPMETGVTQTPRHLVMGMTNKKSLKCEQHLGHN | 2578 |
| WT signal | AMYWYKQSAKKPLELMFVYSLEERVENNSVPSRFSPECPNSSHLFLHLHTLQ | |
| peptide, Cβ | PEDSALYLCASSQDVTSEWVDTIYFGEGSWLTVVEDLRNVTPPKVSLFEPSK | |
| (substituted) | AEIANKQKATLVCLARGFFPDHVELSWWVNGKEVHSGVCTDPQAYKESNYSY | |
| | CLSSRLRVSATFWHNPRNHFRCQVQFHGLSEEDKWPEGSPKPVTQNISAEAW | |
| | GRADCGITSASYQQGVLSATILYEILLGKATLYAVLVSTLVVMAMVKRKNS | |
| β chain with | MACRLLCCAVLCLLGAGELVPMETGVTQTPRHLVMGMTNKKSLKCEQHLGHN | 2579 |
| alternative | AMYWYKQSAKKPLELMFVYSLEERVENNSVPSRFSPECPNSSHLFLHLHTLQ | |
| signal peptide, | PEDSALYLCASSQDVTSEWVDTIYFGEGSWLTVVEDLRNVTPPKVSLFEPSK | |
| Cβ | AEIANKQKATLVCLARGFFPDHVELSWWVNGKEVHSGVCTDPQAYKESNYSY | |
| (substituted) | CLSSRLRVSATFWHNPRNHFRCQVQFHGLSEEDKWPEGSPKPVTQNISAEAW | |
| | GRADCGITSASYQQGVLSATILYEILLGKATLYAVLVSTLVVMAMVKRKNS | |
| β chain with | MHCRLLCCAVLCLLGAGELVPMETGVTQTPRHLVMGMTNKKSLKCEQHLGHN | 2580 |
| alternative | AMYWYKQSAKKPLELMFVYSLEERVENNSVPSRFSPECPNSSHLFLHLHTLQ | |
| signal peptide, | PEDSALYLCASSQDVTSEWVDTIYFGEGSWLTVVEDLRNVTPPKVSLFEPSK | |
| Cβ | AEIANKQKATLVCLARGFFPDHVELSWWVNGKEVHSGVCTDPQAYKESNYSY | |
| (substituted) | CLSSRLRVSATFWHNPRNHERCQVQFHGLSEEDKWPEGSPKPVTQNISAEAW | |
| | GRADCGITSASYQQGVLSATILYEILLGKATLYAVLVSTLVVMAMVKRKNS | |

In some embodiments, TCR058 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR058 interacts with the neoantigen in the context of HLA-C*01:02, as described in International Publication No. WO 2021/163477, incorporated herein by reference in its entirety.

TABLE 6BG
Amino acid sequences of TCR059.

| | | SEQ |
|---|---|---|
| | | ID |
| Description | Sequence | NO: |
| CDR1α | NSASQS | 1581 |
| CDR2α | VYSSGN | 1582 |
| CDR3α | VVDDQTGANNLF | 1583 |
| Vα without | QRKEVEQDPGPFNVPEGATVAFNCTYSNSASQSFFWYRQDCRKEPKLLMSVY | 1584 |
| signal peptide | SSGNEDGRFTAQLNRASQYISLLIRDSKLSDSATYLCVVDDQTGANNLFFGT | |
| (SignalP) | GTRLTVIP | |
| Vα without | RKEVEQDPGPFNVPEGATVAFNCTYSNSASQSFFWYRQDCRKEPKLLMSVYS | 1585 |
| signal peptide | SGNEDGRFTAQLNRASQYISLLIRDSKLSDSATYLCVVDDQTGANNLFFGTG | |
| (IMGT) | TRLTVIP | |
| Vα | MXSLRVLLVILWLQLSWVWSQRKEVEQDPGPFNVPEGATVAFNCTYSNSASQ | 1586 |
| | SFFWYRQDCRKEPKLLMSVYSSGNEDGRFTAQLNRASQYISLLIRDSKLSDS | 1587 |
| | ATYLCVVDDQTGANNLFFGTGTRLTVIP | |
| | (X = any amino acid) | |
| α chain with | MISLRVLLVILWLQLSWVWSQRKEVEQDPGPFNVPEGATVAFNCTYSNSASQ | 1588 |
| WT signal | SFFWYRQDCRKEPKLLMSVYSSGNEDGRFTAQLNRASQYISLLIRDSKLSDS | |
| peptide, Cα | ATYLCVVDDQTGANNLFFGTGTRLTVIPNIQNPEPAVYQLKDPRSQDSTLCL | |
| (substituted) | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| α chain with | MASLRVLLVILWLQLSWVWSQRKEVEQDPGPFNVPEGATVAFNCTYSNSASQ | 1589 |
| alternative | SFFWYRQDCRKEPKLLMSVYSSGNEDGRFTAQLNRASQYISLLIRDSKLSDS | |
| signal peptide, | ATYLCVVDDQTGANNLFFGTGTRLTVIPNIQNPEPAVYQLKDPRSQDSTLCL | |
| Cα | FTDFDSQINVPKTMESGTFITDKCVLDMKAMDSKSNGAIAWSNQTSFTCQDI | |
| (substituted) | FKETNATYPSSDVPCDATLTEKSFETDMNLNFQNLLVIVLRILLLKVAGENL | |
| | LMTLRLWSS | |
| α chain with | MHSLRVLLVILWLQLSWVWSQRKEVEQDPGPFNVPEGATVAFNCTYSNSASQ | 1590 |
| alternative | SFFWYRQDCRKEPKLLMSVYSSGNEDGRFTAQLNRASQYISLLIRDSKLSDS | |
| signal peptide, | ATYLCVVDDQTGANNLFFGTGTRLTVIPNIQNPEPAVYQLKDPRSQDSTLCL | |
| Cα | FTDFDSQINVPKTMESGTFITDKCVLDMKAMDSKSNGAIAWSNQTSFTCQDI | |
| (substituted) | FKETNATYPSSDVPCDATLTEKSFETDMNLNFQNLLVIVLRILLLKVAGENL | |
| | LMTLRLWSS | |
| CDR1β | MNHEY | 2581 |
| CDR2β | SVGAGI | 2582 |
| CDR3β | ASRNLGDTQY | 2583 |
| Vβ without | GVTQTPKFQVLKTGQSMTLQCAQDMNHEYMSWYRQDPGMGLRLIHYSVGAGI | 2584 |
| signal peptide | TDQGEVPNGYNVSRSTTEDFPLRLLSAAPSQTSVYFCASRNLGDTQYFGPGT | |
| (SignalP) | RLTVL | |
| Vβ without | NAGVTQTPKFQVLKTGQSMTLQCAQDMNHEYMSWYRQDPGMGLRLIHYSVGA | 2585 |
| signal peptide | GITDQGEVPNGYNVSRSTTEDFPLRLLSAAPSQTSVYFCASRNLGDTQYFGP | |
| (IMGT) | GTRLTVL | |
| Vβ | MXIGLLCCAALSLLWAGPVNAGVTQTPKFQVLKTGQSMTLQCAQDMNHEYMS | 2586 |
| | WYRQDPGMGLRLIHYSVGAGITDQGEVPNGYNVSRSTTEDEPLRLLSAAPSQ | 2587 |
| | TSVYFCASRNLGDTQYFGPGTRLTVL | |
| | (X = any amino acid) | |
| β chain with | MSIGLLCCAALSLLWAGPVNAGVTQTPKFQVLKTGQSMTLQCAQDMNHEYMS | 2588 |
| WT signal | WYRQDPGMGLRLIHYSVGAGITDQGEVPNGYNVSRSTTEDFPLRLLSAAPSQ | |
| peptide, Cβ | TSVYFCASRNLGDTQYFGPGTRLTVLEDLRNVTPPKVSLFEPSKAEIANKQK | |
| (substituted) | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain with | MAIGLLCCAALSLLWAGPVNAGVTQTPKFQVLKTGQSMTLQCAQDMNHEYMS | 2589 |
| alternative | WYRQDPGMGLRLIHYSVGAGITDQGEVPNGYNVSRSTTEDFPLRLLSAAPSQ | |
| signal peptide, | TSVYFCASRNLGDTQYFGPGTRLTVLEDLRNVTPPKVSLFEPSKAEIANKQK | |
| Cβ | ATLVCLARGFFPDHVELSWWVNGKEVHSGVCTDPQAYKESNYSYCLSSRLRV | |
| (substituted) | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| β chain with | MHIGLLCCAALSLLWAGPVNAGVTQTPKFQVLKTGQSMTLQCAQDMNHEYMS | 2590 |
| alternative | WYRQDPGMGLRLIHYSVGAGITDQGEVPNGYNVSRSTTEDFPLRLLSAAPSQ | |
| signal peptide, | TSVYFCASRNLGDTQYFGPGTRLTVLEDLRNVTPPKVSLFEPSKAEIANKQK | |
| Cβ | ATLVCLARGFFPDHVELSWWVNGKEVHSGVCTDPQAYKESNYSYCLSSRLRV | |
| (substituted) | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |

In some embodiments, TCR059 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR059 interacts with the neoantigen in the context of HLA-C*01:02, as described in International Publication No. WO 2021/163477, incorporated herein by reference in its entirety.

TABLE 6BH
Amino acid sequences of TCR060.

| | | SEQ |
|---|---|---|
| | | ID |
| Description | Sequence | NO: |
| CDR1α | TSINN | 1591 |
| CDR2α | IRSNERE | 1592 |
| CDR3α | ATDGETSGSRLT | 1593 |
| Vα without | QQGEEDPQALSIQEGENATMNCSYKTSINNLQWYRQNSGRGLVHLILIRSNE | 1594 |
| signal peptide | REKHSGRLRVTLDTSKKSSSLLITASRAADTASYFCATDGETSGSRLTFGEG | |
| (SignalP) | TQLTVNP | |
| Vα without | SQQGEEDPQALSIQEGENATMNCSYKTSINNLQWYRQNSGRGLVHLILIRSN | 1595 |
| signal peptide | EREKHSGRLRVTLDTSKKSSSLLITASRAADTASYFCATDGETSGSRLTFGE | |
| (IMGT) | GTQLTVNP | |
| Vα | MXTLLGVSLVILWLQLAVNSQQGEEDPQALSIQEGENATMNCSYKTSINNLQ | 1596 |
| | WYRQNSGRGLVHLILIRSNEREKHSGRLRVTLDTSKKSSSLLITASRAADTA | 1597 |
| | SYFCATDGETSGSRLTFGEGTQLTVNP | |
| | (X = any amino acid) | |
| α chain with | METLLGVSLVILWLQLAVNSQQGEEDPQALSIQEGENATMNCSYKTSINNLQ | 1598 |
| WT signal | WYRQNSGRGLVHLILIRSNEREKHSGRLRVTLDTSKKSSSLLITASRAADTA | |
| peptide, Cα | SYFCATDGETSGSRLTFGEGTQLTVNPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGENLL | |
| | MTLRLWSS | |
| α chain with | MATLLGVSLVILWLQLAVNSQQGEEDPQALSIQEGENATMNCSYKTSINNLQ | 1599 |
| alternative | WYRQNSGRGLVHLILIRSNEREKHSGRLRVTLDTSKKSSSLLITASRAADTA | |
| signal peptide, | SYFCATDGETSGSRLTFGEGTQLTVNPNIQNPEPAVYQLKDPRSQDSTLCLF | |
| Cα | TDFDSQINVPKTMESGTFITDKCVLDMKAMDSKSNGAIAWSNQTSFTCQDIE | |
| (substituted) | KETNATYPSSDVPCDATLTEKSFETDMNLNFQNLLVIVLRILLLKVAGENLL | |
| | MTLRLWSS | |
| α chain with | MHTLLGVSLVILWLQLAVNSQQGEEDPQALSIQEGENATMNCSYKTSINNLQ | 1600 |
| alternative | WYRQNSGRGLVHLILIRSNEREKHSGRLRVTLDTSKKSSSLLITASRAADTA | |
| signal peptide, | SYFCATDGETSGSRLTFGEGTQLTVNPNIQNPEPAVYQLKDPRSQDSTLCLF | |
| Cα | TDFDSQINVPKTMESGTFITDKCVLDMKAMDSKSNGAIAWSNQTSFTCQDIF | |
| (substituted) | KETNATYPSSDVPCDATLTEKSFETDMNLNFQNLLVIVLRILLLKVAGENLL | |
| | MTLRLWSS | |
| CDR1β | DFQATT | 2591 |
| CDR2β | SNEGSKA | 2592 |
| CDR3β | SASRGATGQPQH | 2593 |
| Vβ without | GSGLGAVVSQHPSWVICKSGTSVKIECRSLDFQATTMFWYRQFPKQSLMLMA | 2594 |
| signal peptide | TSNEGSKATYEQGVEKDKFLINHASLTLSTLTVTSAHPEDSSFYICSASRGA | |
| (SignalP) | TGQPQHFGDGTRLSIL | |
| Vβ without | GAVVSQHPSWVICKSGTSVKIECRSLDFQATTMFWYRQFPKQSLMLMATSNE | 2595 |
| signal peptide | GSKATYEQGVEKDKFLINHASLTLSTLTVTSAHPEDSSFYICSASRGATGQP | |
| (IMGT) | QHFGDGTRLSIL | |
| Vβ | MXLLLLLLGPGISLLLPGSLAGSGLGAVVSQHPSWVICKSGTSVKIECRSLD | 2596 |
| | FQATTMFWYRQFPKQSLMLMATSNEGSKATYEQGVEKDKFLINHASLTLSTL | 2597 |
| | TVTSAHPEDSSFYICSASRGATGQPQHFGDGTRLSIL | |
| | (X = any amino acid) | |
| β chain with | MLLLLLLLGPGISLLLPGSLAGSGLGAVVSQHPSWVICKSGTSVKIECRSLD | 2598 |
| WT signal | FQATTMFWYRQFPKQSLMLMATSNEGSKATYEQGVEKDKFLINHASLTLSTL | |
| peptide, Cβ | TVTSAHPEDSSFYICSASRGATGQPQHFGDGTRLSILEDLRNVTPPKVSLFE | |
| (substituted) | PSKAEIANKQKATLVCLARGFFPDHVELSWWVNGKEVHSGVCTDPQAYKESN | |
| | YSYCLSSRLRVSATFWHNPRNHFRCQVQFHGLSEEDKWPEGSPKPVTQNISA | |
| | EAWGRADCGITSASYQQGVLSATILYEILLGKATLYAVLVSTLVVMAMVKRK | |
| | NS | |
| β chain with | MALLLLLLGPGISLLLPGSLAGSGLGAVVSQHPSWVICKSGTSVKIECRSLD | 2599 |
| alternative | FQATTMFWYRQFPKQSLMLMATSNEGSKATYEQGVEKDKFLINHASLTLSTL | |
| signal peptide, | TVTSAHPEDSSFYICSASRGATGQPQHFGDGTRLSILEDLRNVTPPKVSLFE | |
| Cβ | PSKAEIANKQKATLVCLARGFFPDHVELSWWVNGKEVHSGVCTDPQAYKESN | |
| (substituted) | YSYCLSSRLRVSATFWHNPRNHFRCQVQFHGLSEEDKWPEGSPKPVTQNISA | |
| | EAWGRADCGITSASYQQGVLSATILYEILLGKATLYAVLVSTLVVMAMVKRK | |
| | NS | |
| β chain with | MHLLLLLLGPGISLLLPGSLAGSGLGAVVSQHPSWVICKSGTSVKIECRSLD | 2600 |
| alternative | FQATTMFWYRQFPKQSLMLMATSNEGSKATYEQGVEKDKFLINHASLTLSTL | |
| signal peptide, | TVTSAHPEDSSFYICSASRGATGQPQHFGDGTRLSILEDLRNVTPPKVSLFE | |
| Cβ | PSKAEIANKQKATLVCLARGFFPDHVELSWWVNGKEVHSGVCTDPQAYKESN | |
| (substituted) | YSYCLSSRLRVSATFWHNPRNHFRCQVQFHGLSEEDKWPEGSPKPVTQNISA | |
| | EAWGRADCGITSASYQQGVLSATILYEILLGKATLYAVLVSTLVVMAMVKRK | |
| | NS | |

In some embodiments, TCR060 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR060 interacts with the neoantigen in the context of an HLA-DPA1*01:03 chain and an HLA-DPB1*03:01 chain, as described in International Publication No. WO 2021/173902, incorporated herein by reference in its entirety.

TABLE 6BI
Amino acid sequences of TCR061.

| | | SEQ |
|---|---|---|
| | | ID |
| Description | Sequence | NO: |
| CDR1α | SSNFYA | 1601 |
| CDR2α | MTLNGDE | 1602 |
| CDR3α | AFTTGNQFY | 1603 |
| Vα w/o | ILNVEQSPQSLHVQEGDSTNFTCSFPSSNFYALHWYRWETAKSPEALFVMTL | 1604 |
| signal peptide | NGDEKKKGRISATLNTKEGYSYLYIKGSQPEDSATYLCAFTTGNQFYFGTGT | |
| (SignalP) | SLTVIP | |
| Vα w/o | ILNVEQSPQSLHVQEGDSTNFTCSFPSSNFYALHWYRWETAKSPEALFVMTL | 1605 |
| signal peptide | NGDEKKKGRISATLNTKEGYSYLYIKGSQPEDSATYLCAFTTGNQFYFGTGT | |
| (IMGT) | SLTVIP | |
| Vα | MXKNPLAAPLLILWFHLDCVSSILNVEQSPQSLHVQEGDSTNFTCSFPSSNF | 1606 |
| | YALHWYRWETAKSPEALFVMTLNGDEKKKGRISATLNTKEGYSYLYIKGSQP | 1607 |
| | EDSATYLCAFTTGNQFYFGTGTSLTVIP | |
| | (X = any amino acid) | |
| α chain w/ | MEKNPLAAPLLILWFHLDCVSSILNVEQSPQSLHVQEGDSTNFTCSFPSSNF | 1608 |
| WT signal | YALHWYRWETAKSPEALFVMTLNGDEKKKGRISATLNTKEGYSYLYIKGSQP | |
| peptide, Cα | EDSATYLCAFTTGNQFYFGTGTSLTVIPNIQNPEPAVYQLKDPRSQDSTLCL | |
| (substituted) | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGENL | |
| | LMTLRLWSS | |
| α chain w/ | MAKNPLAAPLLILWFHLDCVSSILNVEQSPQSLHVQEGDSTNFTCSFPSSNF | 1609 |
| alternative | YALHWYRWETAKSPEALFVMTLNGDEKKKGRISATLNTKEGYSYLYIKGSQP | |
| signal peptide, | EDSATYLCAFTTGNQFYFGTGTSLTVIPNIQNPEPAVYQLKDPRSQDSTLCL | |
| Cα | FTDFDSQINVPKTMESGTFITDKCVLDMKAMDSKSNGAIAWSNQTSFTCQDI | |
| (substituted) | FKETNATYPSSDVPCDATLTEKSFETDMNLNFQNLLVIVLRILLLKVAGENL | |
| | LMTLRLWSS | |
| α chain w/ | MHKNPLAAPLLILWFHLDCVSSILNVEQSPQSLHVQEGDSTNFTCSFPSSNF | 1610 |
| alternative | YALHWYRWETAKSPEALFVMTLNGDEKKKGRISATLNTKEGYSYLYIKGSQP | |
| signal peptide, | EDSATYLCAFTTGNQFYFGTGTSLTVIPNIQNPEPAVYQLKDPRSQDSTLCL | |
| Cα | FTDFDSQINVPKTMESGTFITDKCVLDMKAMDSKSNGAIAWSNQTSFTCQDI | |
| (substituted) | FKETNATYPSSDVPCDATLTEKSFETDMNLNFQNLLVIVLRILLLKVAGENL | |
| | LMTLRLWSS | |
| CDR1β | SGHDYL | 2601 |
| CDR2β | FNNNVP | 2602 |
| CDR3β | ASSSYGGYSNQPQH | 2603 |
| Vβ w/o | GVIQSPRHEVTEMGQEVTLRCKPISGHDYLFWYRQTMMRGLELLIYENNNVP | 2604 |
| signal peptide | IDDSGMPEDRFSAKMPNASFSTLKIQPSEPRDSAVYFCASSSYGGYSNQPQH | |
| (SignalP) | FGDGTRLSIL | |
| Vβ w/o | DAGVIQSPRHEVTEMGQEVTLRCKPISGHDYLFWYRQTMMRGLELLIYENNN | 2605 |
| signal peptide | VPIDDSGMPEDRFSAKMPNASFSTLKIQPSEPRDSAVYFCASSSYGGYSNQP | |
| (IMGT) | QHFGDGTRLSIL | |
| Vβ | MXSWTLCCVSLCILVAKHTDAGVIQSPRHEVTEMGQEVTLRCKPISGHDYLF | 2606 |
| | WYRQTMMRGLELLIYFNNNVPIDDSGMPEDRFSAKMPNASFSTLKIQPSEPR | 2607 |
| | DSAVYFCASSSYGGYSNQPQHFGDGTRLSIL | |
| | (X = any amino acid) | |
| β chain w/ | MGSWTLCCVSLCILVAKHTDAGVIQSPRHEVTEMGQEVTLRCKPISGHDYLF | 2608 |
| WT signal | WYRQTMMRGLELLIYFNNNVPIDDSGMPEDRFSAKMPNASFSTLKIQPSEPR | |
| peptide, Cβ | DSAVYFCASSSYGGYSNQPQHFGDGTRLSILEDLRNVTPPKVSLFEPSKAEI | |
| (substituted) | ANKQKATLVCLARGFFPDHVELSWWVNGKEVHSGVCTDPQAYKESNYSYCLS | |
| | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |
| β chain w/ | MASWTLCCVSLCILVAKHTDAGVIQSPRHEVTEMGQEVTLRCKPISGHDYLF | 2609 |
| alternative | WYRQTMMRGLELLIYENNNVPIDDSGMPEDRFSAKMPNASESTLKIQPSEPR | |
| signal peptide, | DSAVYFCASSSYGGYSNQPQHFGDGTRLSILEDLRNVTPPKVSLFEPSKAEI | |
| Cβ | ANKQKATLVCLARGFFPDHVELSWWVNGKEVHSGVCTDPQAYKESNYSYCLS | |
| (substituted) | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |
| β chain w/ | MHSWTLCCVSLCILVAKHTDAGVIQSPRHEVTEMGQEVTLRCKPISGHDYLF | 2610 |
| alternative | WYRQTMMRGLELLIYENNNVPIDDSGMPEDRESAKMPNASESTLKIQPSEPR | |
| signal peptide, | DSAVYFCASSSYGGYSNQPQHFGDGTRLSILEDLRNVTPPKVSLFEPSKAEI | |
| Cβ | ANKQKATLVCLARGFFPDHVELSWWVNGKEVHSGVCTDPQAYKESNYSYCLS | |
| (substituted) | SRLRVSATFWHNPRNHFRCQVQFHGLSEEDKWPEGSPKPVTQNISAEAWGRA | |
| | DCGITSASYQQGVLSATILYEILLGKATLYAVLVSTLVVMAMVKRKNS | |

In some embodiments, TCR061 interacts with and/or is specific for tumor protein KRAS (KRAS). In some embodiments, the peptide is from a neoantigen of KRAS. In some embodiments, the neoantigen has the amino acid change G12C relative to the wild type KRAS sequence. In some embodiments, TCR061 interacts with the neoantigen in the context of HLA-DRB1*11:01 as described in International Publication No. WO 2019/060349, incorporated herein by reference in its entirety.

TABLE 6BJ
Amino acid sequences of TCR062.

| | | SEQ |
|---|---|---|
| | | ID |
| Description | Sequence | NO: |
| CDR1α | NIATNDY | 1611 |
| CDR2α | GYKTK | 1612 |
| CDR3α | LVGDMDQAGTALI | 1613 |
| Vα w/o | KTTQPISMDSYEGQEVNITCSHNNIATNDYITWYQQFPSQGPRFIIQGYKTK | 1614 |
| signal peptide | VTNEVASLFIPADRKSSTLSLPRVSLSDTAVYYCLVGDMDQAGTALIFGKGT | |
| (SignalP) | TLSVSS | |
| Vα w/o | LAKTTQPISMDSYEGQEVNITCSHNNIATNDYITWYQQFPSQGPRFIIQGYK | 1615 |
| signal peptide | TKVTNEVASLFIPADRKSSTLSLPRVSLSDTAVYYCLVGDMDQAGTALIFGK | |
| (IMGT) | GTTLSVSS | |
| Vα | MXQVARVIVELTLSTLSLAKTTQPISMDSYEGQEVNITCSHNNIATNDYITW | 1616 |
| | YQQFPSQGPRFIIQGYKTKVTNEVASLFIPADRKSSTLSLPRVSLSDTAVYY | 1617 |
| | CLVGDMDQAGTALIFGKGTTLSVSS | |
| | (X = any amino acid) | |
| α chain w/ | MRQVARVIVELTLSTLSLAKTTQPISMDSYEGQEVNITCSHNNIATNDYITW | 1618 |
| WT signal | YQQFPSQGPRFIIQGYKTKVTNEVASLFIPADRKSSTLSLPRVSLSDTAVYY | |
| peptide, Cα | CLVGDMDQAGTALIFGKGTTLSVSSNIQNPEPAVYQLKDPRSQDSTLCLFTD | |
| (substituted) | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| α chain w/ | MAQVARVIVELTLSTLSLAKTTQPISMDSYEGQEVNITCSHNNIATNDYITW | 1619 |
| alternative | YQQFPSQGPRFIIQGYKTKVTNEVASLFIPADRKSSTLSLPRVSLSDTAVYY | |
| signal peptide, | CLVGDMDQAGTALIFGKGTTLSVSSNIQNPEPAVYQLKDPRSQDSTLCLFTD | |
| Cα | FDSQINVPKTMESGTFITDKCVLDMKAMDSKSNGAIAWSNQTSFTCQDIFKE | |
| (substituted) | TNATYPSSDVPCDATLTEKSFETDMNLNFQNLLVIVLRILLLKVAGENLLMT | |
| | LRLWSS | |
| α chain w/ | MHQVARVIVELTLSTLSLAKTTQPISMDSYEGQEVNITCSHNNIATNDYITW | 1620 |
| alternative | YQQFPSQGPRFIIQGYKTKVTNEVASLFIPADRKSSTLSLPRVSLSDTAVYY | |
| signal peptide, | CLVGDMDQAGTALIFGKGTTLSVSSNIQNPEPAVYQLKDPRSQDSTLCLFTD | |
| Cα | FDSQINVPKTMESGTFITDKCVLDMKAMDSKSNGAIAWSNQTSFTCQDIFKE | |
| (substituted) | TNATYPSSDVPCDATLTEKSFETDMNLNFQNLLVIVLRILLLKVAGENLLMT | |
| | LRLWSS | |
| CDR1β | SGHDT | 2611 |
| CDR2β | YYEEEE | 2612 |
| CDR3β | ASSLGEGRVDGYT | 2613 |
| Vβ w/o | GVTQSPTHLIKTRGQQVTLRCSPKSGHDTVSWYQQALGQGPQFIFQYYEEEE | 2614 |
| signal peptide | RQRGNFPDRFSGHQFPNYSSELNVNALLLGDSALYLCASSLGEGRVDGYTFG | |
| (SignalP) | SGTRLTVV | |
| Vβ w/o | DAGVTQSPTHLIKTRGQQVTLRCSPKSGHDTVSWYQQALGQGPQFIFQYYEE | 2615 |
| signal peptide | EERQRGNFPDRFSGHQFPNYSSELNVNALLLGDSALYLCASSLGEGRVDGYT | |
| (IMGT) | FGSGTRLTVV | |
| Vβ | MXPGLLCWALLCLLGAGLVDAGVTQSPTHLIKTRGQQVTLRCSPKSGHDTVS | 2616 |
| | WYQQALGQGPQFIFQYYEEEERQRGNFPDRFSGHQFPNYSSELNVNALLLGD | |
| | SALYLCASSLGEGRVDGYTFGSGTRLTVV | 2617 |
| | (X = any amino acid) | |
| β chain w/ | MGPGLLCWALLCLLGAGLVDAGVTQSPTHLIKTRGQQVTLRCSPKSGHDTVS | 2618 |
| WT signal | WYQQALGQGPQFIFQYYEEEERQRGNFPDRFSGHQFPNYSSELNVNALLLGD | |
| peptide, Cβ | SALYLCASSLGEGRVDGYTFGSGTRLTVVEDLRNVTPPKVSLFEPSKAEIAN | |
| (substituted) | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/ | MAPGLLCWALLCLLGAGLVDAGVTQSPTHLIKTRGQQVTLRCSPKSGHDTVS | 2619 |
| alternative | WYQQALGQGPQFIFQYYEEEERQRGNFPDRESGHQFPNYSSELNVNALLLGD | |
| signal peptide, | SALYLCASSLGEGRVDGYTFGSGTRLTVVEDLRNVTPPKVSLFEPSKAEIAN | |
| Cβ | KQKATLVCLARGFFPDHVELSWWVNGKEVHSGVCTDPQAYKESNYSYCLSSR | |
| (substituted) | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/ | MHPGLLCWALLCLLGAGLVDAGVTQSPTHLIKTRGQQVTLRCSPKSGHDTVS | 2620 |
| alternative | WYQQALGQGPQFIFQYYEEEERQRGNFPDRESGHQFPNYSSELNVNALLLGD | |
| signal peptide, | SALYLCASSLGEGRVDGYTFGSGTRLTVVEDLRNVTPPKVSLFEPSKAEIAN | |
| Cβ | KQKATLVCLARGFFPDHVELSWWVNGKEVHSGVCTDPQAYKESNYSYCLSSR | |
| (substituted) | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR062 interacts with and/or is specific for KRAS. In some embodiments, the peptide is from a neoantigen of KRAS. In some embodiments, the neoantigen has the amino acid change G12D relative to the wild type KRAS sequence. In some embodiments, TCR062 interacts with the neoantigen in the context of HLA-C*08:02 as described in International Publication No. WO 2018/026691, incorporated herein by reference in its entirety.

TABLE 6BK
Amino acid sequences of TCR063.

| | | SEQ |
|---|---|---|
| | | ID |
| Description | Sequence | NO: |
| CDR1α | NIATNDY | 1621 |
| CDR2α | GYKTK | 1622 |
| CDR3α | LVGDMDQAGTALI | 1623 |
| Vα w/o | KTTQPISMDSYEGQEVNITCSHNNIATNDYITWYQQFPSQGPRFIIQGYKTK | 1624 |
| signal peptide | VTNEVASLFIPADRKSSTLSLPRVSLSDTAVYYCLVGDMDQAGTALIFGKGT | |
| (SignalP) | TLSVSS | |
| Vα w/o | LAKTTQPISMDSYEGQEVNITCSHNNIATNDYITWYQQFPSQGPRFIIQGYK | 1625 |
| signal peptide | TKVTNEVASLFIPADRKSSTLSLPRVSLSDTAVYYCLVGDMDQAGTALIFGK | |
| (IMGT) | GTTLSVSS | |
| Vα | MXQVARVIVFLTLSTLSLAKTTQPISMDSYEGQEVNITCSHNNIATNDYITW | 1626 |
| | YQQFPSQGPRFIIQGYKTKVTNEVASLFIPADRKSSTLSLPRVSLSDTAVYY | 1627 |
| | CLVGDMDQAGTALIFGKGTTLSVSS | |
| | (X = any amino acid) | |
| α chain w/ | MRQVARVIVELTLSTLSLAKTTQPISMDSYEGQEVNITCSHNNIATNDYITW | 1628 |
| WT signal | YQQFPSQGPRFIIQGYKTKVTNEVASLFIPADRKSSTLSLPRVSLSDTAVYY | |
| peptide, Cα | CLVGDMDQAGTALIFGKGTTLSVSSNIQNPEPAVYQLKDPRSQDSTLCLFTD | |
| (substituted) | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGENLLMT | |
| | LRLWSS | |
| α chain w/ | MAQVARVIVELTLSTLSLAKTTQPISMDSYEGQEVNITCSHNNIATNDYITW | 1629 |
| alternative | YQQFPSQGPRFIIQGYKTKVTNEVASLFIPADRKSSTLSLPRVSLSDTAVYY | |
| signal peptide, | CLVGDMDQAGTALIFGKGTTLSVSSNIQNPEPAVYQLKDPRSQDSTLCLFTD | |
| Cα | FDSQINVPKTMESGTFITDKCVLDMKAMDSKSNGAIAWSNQTSFTCQDIFKE | |
| (substituted) | TNATYPSSDVPCDATLTEKSFETDMNLNFQNLLVIVLRILLLKVAGENLLMT | |
| | LRLWSS | |
| α chain w/ | MHQVARVIVFLTLSTLSLAKTTQPISMDSYEGQEVNITCSHNNIATNDYITW | 1630 |
| alternative | YQQFPSQGPRFIIQGYKTKVTNEVASLFIPADRKSSTLSLPRVSLSDTAVYY | |
| signal peptide, | CLVGDMDQAGTALIFGKGTTLSVSSNIQNPEPAVYQLKDPRSQDSTLCLFTD | |
| Cα | FDSQINVPKTMESGTFITDKCVLDMKAMDSKSNGAIAWSNQTSFTCQDIFKE | |
| (substituted) | TNATYPSSDVPCDATLTEKSFETDMNLNFQNLLVIVLRILLLKVAGENLLMT | |
| | LRLWSS | |
| CDR1β | SGHDT | 2621 |
| CDR2β | YYEEEE | 2622 |
| CDR3β | ASSLGRASNQPQH | 2623 |
| Vβ w/o | GVTQSPTHLIKTRGQQVTLRCSPKSGHDTVSWYQQALGQGPQFIFQYYEEEE | 2624 |
| signal peptide | RQRGNFPDRESGHQFPNYSSELNVNALLLGDSALYLCASSLGRASNQPQHFG | |
| (SignalP) | DGTRLSIL | |
| Vβ w/o | DAGVTQSPTHLIKTRGQQVTLRCSPKSGHDTVSWYQQALGQGPQFIFQYYEE | 2625 |
| signal peptide | EERQRGNFPDRESGHQFPNYSSELNVNALLLGDSALYLCASSLGRASNQPQH | |
| (IMGT) | FGDGTRLSIL | |
| Vβ | MXPGLLCWALLCLLGAGLVDAGVTQSPTHLIKTRGQQVTLRCSPKSGHDTVS | 2626 |
| | WYQQALGQGPQFIFQYYEEEERQRGNFPDRESGHQFPNYSSELNVNALLLGD | 2627 |
| | SALYLCASSLGRASNQPQHFGDGTRLSIL | |
| | (X = any amino acid) | |
| β chain w/ | MGPGLLCWALLCLLGAGLVDAGVTQSPTHLIKTRGQQVTLRCSPKSGHDTVS | 2628 |
| WT signal | WYQQALGQGPQFIFQYYEEEERQRGNFPDRFSGHQFPNYSSELNVNALLLGD | |
| peptide, Cβ | SALYLCASSLGRASNQPQHFGDGTRLSILEDLRNVTPPKVSLFEPSKAEIAN | |
| (substituted) | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/ | MAPGLLCWALLCLLGAGLVDAGVTQSPTHLIKTRGQQVTLRCSPKSGHDTVS | 2629 |
| alternative | WYQQALGQGPQFIFQYYEEEERQRGNFPDRESGHQFPNYSSELNVNALLLGD | |
| signal peptide, | SALYLCASSLGRASNQPQHFGDGTRLSILEDLRNVTPPKVSLFEPSKAEIAN | |
| Cβ | KQKATLVCLARGFFPDHVELSWWVNGKEVHSGVCTDPQAYKESNYSYCLSSR | |
| (substituted) | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| β chain w/ | MHPGLLCWALLCLLGAGLVDAGVTQSPTHLIKTRGQQVTLRCSPKSGHDTVS | 2630 |
| alternative | WYQQALGQGPQFIFQYYEEEERQRGNFPDRESGHQFPNYSSELNVNALLLGD | |
| signal peptide, | SALYLCASSLGRASNQPQHFGDGTRLSILEDLRNVTPPKVSLFEPSKAEIAN | |
| Cβ | KQKATLVCLARGFFPDHVELSWWVNGKEVHSGVCTDPQAYKESNYSYCLSSR | |
| (substituted) | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR063 interacts with and/or is specific for KRAS. In some embodiments, the peptide is from a neoantigen of KRAS. In some embodiments, the neoantigen has the amino acid change G12D relative to the wild type KRAS sequence. In some embodiments, TCR063 interacts with the neoantigen in the context of HLA-C*08:02 as described in International Publication No. WO 2018/026691, incorporated herein by reference in its entirety.

TABLE 6BL
Amino acid sequences of TCR064.

| | | SEQ |
|---|---|---|
| Description | Sequence | ID NO: |
| CDR1α | NIATNDY | 1631 |
| CDR2α | GYKTK | 1632 |
| CDR3α | LVGDRDQAGTALI | 1633 |
| Vα w/o signal peptide | KTTQPISMDSYEGQEVNITCSHNNIATNDYITWYQQFPSQGPRFIIQGYKTK | 1634 |
| (SignalP) | VTNEVASLFIPADRKSSTLSLPRVSLSDTAVYYCLVGDRDQAGTALIFGKGT | |
| | TLSVSS | |
| Vα w/o signal peptide | LAKTTQPISMDSYEGQEVNITCSHNNIATNDYITWYQQFPSQGPRFIIQGYK | 1635 |
| (IMGT) | TKVTNEVASLFIPADRKSSTLSLPRVSLSDTAVYYCLVGDRDQAGTALIFGK | |
| | GTTLSVSS | |
| Vα | MXQVARVIVFLTLSTLSLAKTTQPISMDSYEGQEVNITCSHNNIATNDYITW | 1636 |
| | YQQFPSQGPRFIIQGYKTKVTNEVASLFIPADRKSSTLSLPRVSLSDTAVYY | 1637 |
| | CLVGDRDQAGTALIFGKGTTLSVSS | |
| | (X = any amino acid) | |
| α chain w/WT signal | MRQVARVIVFLTLSTLSLAKTTQPISMDSYEGQEVNITCSHNNIATNDYITW | 1638 |
| peptide, Cα | YQQFPSQGPRFIIQGYKTKVTNEVASLFIPADRKSSTLSLPRVSLSDTAVYY | |
| (substituted) | CLVGDRDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGFNLLMT | |
| | LRLWSS | |
| α chain w/alternative | MAQVARVIVFLTLSTLSLAKTTQPISMDSYEGQEVNITCSHNNIATNDYITW | 1639 |
| signal peptide, Cα | YQQFPSQGPRFIIQGYKTKVTNEVASLFIPADRKSSTLSLPRVSLSDTAVYY | |
| (substituted) | CLVGDRDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGFNLLMT | |
| | LRLWSS | |
| α chain w/alternative | MHQVARVIVFLTLSTLSLAKTTQPISMDSYEGQEVNITCSHNNIATNDYITW | 1640 |
| signal peptide, Cα | YQQFPSQGPRFIIQGYKTKVTNEVASLFIPADRKSSTLSLPRVSLSDTAVYY | |
| (substituted) | CLVGDRDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGFNLLMT | |
| | LRLWSS | |
| CDR1ß | SGHDT | 2631 |
| CDR2ß | YYEEEE | 2632 |
| CDR3ß | ASSFGQSSTYGYT | 2633 |
| Vß w/o signal peptide | GVTQSPTHLIKTRGQQVTLRCSPKSGHDTVSWYQQALGQGPQFIFQYYEEEE | 2634 |
| (SignalP) | RQRGNFPDRFSGHQFPNYSSELNVNALLLGDSALYLCASSFGQSSTYGYTFG | |
| | SGTRLTVV | |
| Vß w/o signal peptide | DAGVTQSPTHLIKTRGQQVTLRCSPKSGHDTVSWYQQALGQGPQFIFQYYEE | 2635 |
| (IMGT) | EERQRGNFPDRFSGHQFPNYSSELNVNALLLGDSALYLCASSFGQSSTYGYT | |
| | FGSGTRLTVV | |
| Vß | MXPGLLCWALLCLLGAGLVDAGVTQSPTHLIKTRGQQVTLRCSPKSGHDTVS | 2636 |
| | WYQQALGQGPQFIFQYYEEEERQRGNFPDRFSGHQFPNYSSELNVNALLLGD | 2637 |
| | SALYLCASSFGQSSTYGYTFGSGTRLTVV | |
| | (X = any amino acid) | |
| ß chain w/WT signal | MGPGLLCWALLCLLGAGLVDAGVTQSPTHLIKTRGQQVTLRCSPKSGHDTVS | 2638 |
| peptide, Cß | WYQQALGQGPQFIFQYYEEEERQRGNFPDRFSGHQFPNYSSELNVNALLLGD | |
| (substituted) | SALYLCASSFGQSSTYGYTFGSGTRLTVVEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| ß chain w/alternative | MAPGLLCWALLCLLGAGLVDAGVTQSPTHLIKTRGQQVTLRCSPKSGHDTVS | 2639 |
| signal peptide, Cß | WYQQALGQGPQFIFQYYEEEERQRGNFPDRFSGHQFPNYSSELNVNALLLGD | |
| (substituted) | SALYLCASSFGQSSTYGYTFGSGTRLTVVEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| ß chain w/alternative | MHPGLLCWALLCLLGAGLVDAGVTQSPTHLIKTRGQQVTLRCSPKSGHDTVS | 2640 |
| signal peptide, Cß | WYQQALGQGPQFIFQYYEEEERQRGNFPDRFSGHQFPNYSSELNVNALLLGD | |
| (substituted) | SALYLCASSFGQSSTYGYTFGSGTRLTVVEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR064 interacts with and/or is specific for KRAS. In some embodiments, the peptide is from a neoantigen of KRAS. In some embodiments, the neoantigen has the amino acid change G12D relative to the wild type KRAS sequence. In some embodiments, TCR064 interacts with the neoantigen in the context of HLA-C*08:02 as described in International Publication No. WO 2018/026691, incorporated herein by reference in its entirety.

TABLE 6BM
Amino acid sequences of TCR065.

| | | SEQ |
|---|---|---|
| Description | Sequence | ID NO: |
| CDR1α | NIATNDY | 1641 |
| CDR2α | GYKTK | 1642 |
| CDR3α | LVGDMDQAGTALI | 1643 |
| Vα w/o signal peptide | KTTQPISMDSYEGQEVNITCSHNNIATNDYITWYQQFPSQGPRFIIQGYKTK | 1644 |
| (SignalP) | VTNEVASLFIPADRKSSTLSLPRVSLSDTAVYYCLVGDMDQAGTALIFGKGT | |
| | TLSVSS | |
| Vα w/o signal peptide | LAKTTQPISMDSYEGQEVNITCSHNNIATNDYITWYQQFPSQGPRFIIQGYK | 1645 |
| (IMGT) | TKVTNEVASLFIPADRKSSTLSLPRVSLSDTAVYYCLVGDMDQAGTALIFGK | |
| | GTTLSVSS | |
| Vα | MXQVARVIVFLTLSTLSLAKTTQPISMDSYEGQEVNITCSHNNIATNDYITW | 1646 |
| | YQQFPSQGPRFIIQGYKTKVTNEVASLFIPADRKSSTLSLPRVSLSDTAVYY | 1647 |
| | CLVGDMDQAGTALIFGKGTTLSVSS | |
| | (X = any amino acid) | |
| α chain w/WT signal | MRQVARVIVFLTLSTLSLAKTTQPISMDSYEGQEVNITCSHNNIATNDYITW | 1648 |
| peptide, Cα | YQQFPSQGPRFIIQGYKTKVTNEVASLFIPADRKSSTLSLPRVSLSDTAVYY | |
| (substituted) | CLVGDMDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGFNLLMT | |
| | LRLWSS | |
| α chain w/alternative | MAQVARVIVFLTLSTLSLAKTTQPISMDSYEGQEVNITCSHNNIATNDYITW | 1649 |
| signal peptide, Cα | YQQFPSQGPRFIIQGYKTKVTNEVASLFIPADRKSSTLSLPRVSLSDTAVYY | |
| (substituted) | CLVGDMDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGFNLLMT | |
| | LRLWSS | |
| α chain w/alternative | MHQVARVIVFLTLSTLSLAKTTQPISMDSYEGQEVNITCSHNNIATNDYITW | 1650 |
| signal peptide, Cα | YQQFPSQGPRFIIQGYKTKVTNEVASLFIPADRKSSTLSLPRVSLSDTAVYY | |
| (substituted) | CLVGDMDQAGTALIFGKGTTLSVSSNIQNPEPAVYQLKDPRSQDSTLCLFTD | |
| | FDSQINVPKTMESGTFITDKCVLDMKAMDSKSNGAIAWSNQTSFTCQDIFKE | |
| | TNATYPSSDVPCDATLTEKSFETDMNLNFQNLLVIVLRILLLKVAGFNLLMT | |
| | LRLWSS | |
| CDR1ß | SGHDT | 2641 |
| CDR2ß | YYEEEE | 2642 |
| CDR3ß | ASSLGQTNYGYT | 2643 |
| Vß w/o signal peptide | GVTQSPTHLIKTRGQQVTLRCSPKSGHDTVSWYQQALGQGPQFIFQYYEEEE | 2644 |
| (SignalP) | RQRGNFPDRFSGHQFPNYSSELNVNALLLGDSALYLCASSLGQTNYGYTFGS | |
| | GTRLTVV | |
| Vß w/o signal peptide | DAGVTQSPTHLIKTRGQQVTLRCSPKSGHDTVSWYQQALGQGPQFIFQYYEE | 2645 |
| (IMGT) | EERQRGNFPDRFSGHQFPNYSSELNVNALLLGDSALYLCASSLGQTNYGYTF | |
| | GSGTRLTVV | |
| Vß | MXPGLLCWALLCLLGAGLVDAGVTQSPTHLIKTRGQQVTLRCSPKSGHDTVS | 2646 |
| | WYQQALGQGPQFIFQYYEEEERQRGNFPDRFSGHQFPNYSSELNVNALLLGD | 2647 |
| | SALYLCASSLGQTNYGYTFGSGTRLTVV | |
| | (X = any amino acid) | |
| ß chain w/WT signal | MGPGLLCWALLCLLGAGLVDAGVTQSPTHLIKTRGQQVTLRCSPKSGHDTVS | 2648 |
| peptide, Cß | WYQQALGQGPQFIFQYYEEEERQRGNFPDRFSGHQFPNYSSELNVNALLLGD | |
| (substituted) | SALYLCASSLGQTNYGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| ß chain w/alternative | MAPGLLCWALLCLLGAGLVDAGVTQSPTHLIKTRGQQVTLRCSPKSGHDTVS | 2649 |
| signal peptide, Cß | WYQQALGQGPQFIFQYYEEEERQRGNFPDRFSGHQFPNYSSELNVNALLLGD | |
| (substituted) | SALYLCASSLGQTNYGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| ß chain w/alternative | MHPGLLCWALLCLLGAGLVDAGVTQSPTHLIKTRGQQVTLRCSPKSGHDTVS | 2650 |
| signal peptide, Cß | WYQQALGQGPQFIFQYYEEEERQRGNFPDRFSGHQFPNYSSELNVNALLLGD | |
| (substituted) | SALYLCASSLGQTNYGYTFGSGTRLTVVEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR065 interacts with and/or is specific for KRAS. In some embodiments, the peptide is from a neoantigen of KRAS. In some embodiments, the neoantigen has the amino acid change G12D relative to the wild type KRAS sequence. In some embodiments, TCR065 interacts with the neoantigen in the context of HLA-Cw*08:02 as described in International Publication No. WO 2017/048593, incorporated herein by reference in its entirety.

TABLE 6BN
Amino acid sequences of TCR066.

| | | SEQ |
|---|---|---|
| Description | Sequence | ID NO: |
| CDR1α | DRGSQS | 1651 |
| CDR2α | IYSNGD | 1652 |
| CDR3α | AAAMDSSYKLI | 1653 |
| Vα w/o signal peptide | QQKEVEQNSGPLSVPEGAIASLNCTYSDRGSQSFFWYRQYSGKSPELIMFIY | 1654 |
| (SignalP) | SNGDKEDGRFTAQLNKASQYVSLLIRDSQPSDSATYLCAAAMDSSYKLIFGS | |
| | GTRLLVRP | |
| Vα w/o signal peptide | QKEVEQNSGPLSVPEGAIASLNCTYSDRGSQSFFWYRQYSGKSPELIMFIYS | 1655 |
| (IMGT) | NGDKEDGRFTAQLNKASQYVSLLIRDSQPSDSATYLCAAAMDSSYKLIFGSG | |
| | TRLLVRP | |
| Vα | MXSLRVLLVILWLQLSWVWSQQKEVEQNSGPLSVPEGAIASLNCTYSDRGSQ | 1656 |
| | SFFWYRQYSGKSPELIMFIYSNGDKEDGRFTAQLNKASQYVSLLIRDSQPSD | 1657 |
| | SATYLCAAAMDSSYKLIFGSGTRLLVRP | |
| | (X = any amino acid) | |
| α chain w/WT signal | MKSLRVLLVILWLQLSWVWSQQKEVEQNSGPLSVPEGAIASLNCTYSDRGSQ | 1658 |
| peptide, Cα | SFFWYRQYSGKSPELIMFIYSNGDKEDGRFTAQLNKASQYVSLLIRDSQPSD | |
| (substituted) | SATYLCAAAMDSSYKLIFGSGTRLLVRPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGFNL | |
| | LMTLRLWSS | |
| α chain w/alternative | MASLRVLLVILWLQLSWVWSQQKEVEQNSGPLSVPEGAIASLNCTYSDRGSQ | 1659 |
| signal peptide, Cα | SFFWYRQYSGKSPELIMFIYSNGDKEDGRFTAQLNKASQYVSLLIRDSQPSD | |
| (substituted) | SATYLCAAAMDSSYKLIFGSGTRLLVRPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGFNL | |
| | LMTLRLWSS | |
| α chain w/alternative | MHSLRVLLVILWLQLSWVWSQQKEVEQNSGPLSVPEGAIASLNCTYSDRGSQ | 1660 |
| signal peptide, Cα | SFFWYRQYSGKSPELIMFIYSNGDKEDGRFTAQLNKASQYVSLLIRDSQPSD | |
| (substituted) | SATYLCAAAMDSSYKLIFGSGTRLLVRPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGFNL | |
| | LMTLRLWSS | |
| CDR1ß | WSHSY | 2651 |
| CDR2ß | SAAADI | 2652 |
| CDR3ß | ASSDPGTEAF | 2653 |
| Vß w/o signal peptide | GITQSPRYKITETGRQVTLMCHQTWSHSYMFWYRQDLGHGLRLIYYSAAADI | 2654 |
| (SignalP) | TDKGEVPDGYVVSRSKTENFPLTLESATRSQTSVYFCASSDPGTEAFFGQGT | |
| | RLTVV | |
| Vß w/o signal peptide | DAGITQSPRYKITETGRQVTLMCHQTWSHSYMFWYRQDLGHGLRLIYYSAAA | 2655 |
| (IMGT) | DITDKGEVPDGYVVSRSKTENFPLTLESATRSQTSVYFCASSDPGTEAFFGQ | |
| | GTRLTVV | |
| Vß | MXTRLFFYVALCLLWAGHRDAGITQSPRYKITETGRQVTLMCHQTWSHSYMF | 2656 |
| | WYRQDLGHGLRLIYYSAAADITDKGEVPDGYVVSRSKTENFPLTLESATRSQ | 2657 |
| | TSVYFCASSDPGTEAFFGQGTRLTVV | |
| | (X = any amino acid) | |
| ß chain w/WT signal | MGTRLFFYVALCLLWAGHRDAGITQSPRYKITETGRQVTLMCHQTWSHSYMF | 2658 |
| peptide, Cß | WYRQDLGHGLRLIYYSAAADITDKGEVPDGYVVSRSKTENFPLTLESATRSQ | |
| (substituted) | TSVYFCASSDPGTEAFFGQGTRLTVVEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| ß chain w/alternative | MATRLFFYVALCLLWAGHRDAGITQSPRYKITETGRQVTLMCHQTWSHSYMF | 2659 |
| signal peptide, Cß | WYRQDLGHGLRLIYYSAAADITDKGEVPDGYVVSRSKTENFPLTLESATRSQ | |
| (substituted) | TSVYFCASSDPGTEAFFGQGTRLTVVEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| ß chain w/alternative | MHTRLFFYVALCLLWAGHRDAGITQSPRYKITETGRQVTLMCHQTWSHSYMF | 2660 |
| signal peptide, Cß | WYRQDLGHGLRLIYYSAAADITDKGEVPDGYVVSRSKTENFPLTLESATRSQ | |
| (substituted) | TSVYFCASSDPGTEAFFGQGTRLTVVEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |

In some embodiments, TCR066 interacts with and/or is specific for KRAS. In some embodiments, the peptide is from a neoantigen of KRAS. In some embodiments, the neoantigen has the amino acid change G12D relative to the wild type KRAS sequence. In some embodiments, TCR066 interacts with the neoantigen in the context of HLA-C*08:02 as described in International Publication No. WO 2018/026691, incorporated herein by reference in its entirety.

TABLE 6BO
Amino acid sequences of TCR067.

| | | SEQ |
|---|---|---|
| Description | Sequence | ID NO: |
| CDR1α | TIYSNPF | 1661 |
| CDR2α | SFTDNKR | 1662 |
| CDR3α | ALRGNAGAKLT | 1663 |
| Vα w/o signal peptide | DGDSVTQTEGLVTLTEGLPVMLNCTYQTIYSNPFLFWYVQHLNESPRLLLKS | 1664 |
| (SignalP) | FTDNKRTEHQGFHATLHKSSSSFHLQKSSAQLSDSALYYCALRGNAGAKLTF | |
| | GGGTRLTVRPD | |
| Vα w/o signal peptide | GDSVTQTEGLVTLTEGLPVMLNCTYQTIYSNPFLFWYVQHLNESPRLLLKSF | 1665 |
| (IMGT) | TDNKRTEHQGFHATLHKSSSSFHLQKSSAQLSDSALYYCALRGNAGAKLTFG | |
| | GGTRLTVRPD | |
| Vα | MXPVTCSVLVLLLMLRRSNGDGDSVTQTEGLVTLTEGLPVMLNCTYQTIYSN | 1666 |
| | PFLFWYVQHLNESPRLLLKSFTDNKRTEHQGFHATLHKSSSSFHLQKSSAQL | 1667 |
| | SDSALYYCALRGNAGAKLTFGGGTRLTVRPD | |
| | (X = any amino acid) | |
| α chain w/WT signal | MRPVTCSVLVLLLMLRRSNGDGDSVTQTEGLVTLTEGLPVMLNCTYQTIYSN | 1668 |
| peptide, Cα | PFLFWYVQHLNESPRLLLKSFTDNKRTEHQGFHATLHKSSSSFHLQKSSAQL | |
| (substituted) | SDSALYYCALRGNAGAKLTFGGGTRLTVRPDIQNPEPAVYQLKDPRSQDSTL | |
| | CLFTDFDSQINVPKTMESGTFITDKTVLDMKAMDSKSNGAIAWSNQTSFTCQ | |
| | DIFKETNATYPSSDVPCDATLTEKSFETDMNLNFQNLSVMGLRILLLKVAGF | |
| | NLLMTLRLWSS | |
| α chain w/alternative | MAPVTCSVLVLLLMLRRSNGDGDSVTQTEGLVTLTEGLPVMLNCTYQTIYSN | 1669 |
| signal peptide, Cα | PFLFWYVQHLNESPRLLLKSFTDNKRTEHQGFHATLHKSSSSFHLQKSSAQL | |
| (substituted) | SDSALYYCALRGNAGAKLTFGGGTRLTVRPDIQNPEPAVYQLKDPRSQDSTL | |
| | CLFTDFDSQINVPKTMESGTFITDKTVLDMKAMDSKSNGAIAWSNQTSFTCQ | |
| | DIFKETNATYPSSDVPCDATLTEKSFETDMNLNFQNLSVMGLRILLLKVAGE | |
| | NLLMTLRLWSS | |
| α chain w/alternative | MHPVTCSVLVLLLMLRRSNGDGDSVTQTEGLVTLTEGLPVMLNCTYQTIYSN | 1670 |
| signal peptide, Cα | PFLFWYVQHLNESPRLLLKSFTDNKRTEHQGFHATLHKSSSSFHLQKSSAQL | |
| (substituted) | SDSALYYCALRGNAGAKLTFGGGTRLTVRPDIQNPEPAVYQLKDPRSQDSTL | |
| | CLFTDFDSQINVPKTMESGTFITDKTVLDMKAMDSKSNGAIAWSNQTSFTCQ | |
| | DIFKETNATYPSSDVPCDATLTEKSFETDMNLNFQNLSVMGLRILLLKVAGF | |
| | NLLMTLRLWSS | |
| CDR1ß | LGHDT | 2661 |
| CDR2ß | YNNKQL | 2662 |
| CDR3ß | ASSSRDWSAETLY | 2663 |
| Vß w/o signal peptide | AVFQTPNYHVTQVGNEVSFNCKQTLGHDTMYWYKQDSKKLLKIMFSYNNKQL | 2664 |
| (SignalP) | IVNETVPRRFSPQSSDKAHLNLRIKSVEPEDSAVYLCASSSRDWSAETLYFG | |
| | SGTRLTVL | |
| Vß w/o signal peptide | ETAVFQTPNYHVTQVGNEVSFNCKQTLGHDTMYWYKQDSKKLLKIMFSYNNK | 2665 |
| (IMGT) | QLIVNETVPRRESPQSSDKAHLNLRIKSVEPEDSAVYLCASSSRDWSAETLY | |
| | FGSGTRLTVL | |
| Vß | MXCRLLSCVAFCLLGIGPLETAVFQTPNYHVTQVGNEVSFNCKQTLGHDTMY | 2666 |
| | WYKQDSKKLLKIMFSYNNKQLIVNETVPRRFSPQSSDKAHLNLRIKSVEPED | 2667 |
| | SAVYLCASSSRDWSAETLYFGSGTRLTVL | |
| | (X = any amino acid) | |
| ß chain w/WT signal | MGCRLLSCVAFCLLGIGPLETAVFQTPNYHVTQVGNEVSFNCKQTLGHDTMY | 2668 |
| peptide, Cß | WYKQDSKKLLKIMFSYNNKQLIVNETVPRRESPQSSDKAHLNLRIKSVEPED | |
| (substituted) | SAVYLCASSSRDWSAETLYFGSGTRLTVLEDLRNVTPPKVSLFEPSKAEIAN | |
| | KQKATLVCLARGFFPDHVELSWWVNGKEVHSGVSTDPQAYKESNYSYCLSSR | |
| | LRVSATFWHNPRNHFRCQVQFHGLSEEDKWPEGSPKPVTQNISAEAWGRADC | |
| | GITSASYHQGVLSATILYEILLGKATLYAVLVSGLVLMAMVKKKNS | |
| ß chain w/alternative | MACRLLSCVAFCLLGIGPLETAVFQTPNYHVTQVGNEVSFNCKQTLGHDTMY | 2669 |
| signal peptide, Cß | WYKQDSKKLLKIMFSYNNKQLIVNETVPRRESPQSSDKAHLNLRIKSVEPED | |
| (substituted) | SAVYLCASSSRDWSAETLYFGSGTRLTVLEDLRNVTPPKVSLFEPSKAEIAN | |
| | KQKATLVCLARGFFPDHVELSWWVNGKEVHSGVSTDPQAYKESNYSYCLSSR | |
| | LRVSATFWHNPRNHFRCQVQFHGLSEEDKWPEGSPKPVTQNISAEAWGRADC | |
| | GITSASYHQGVLSATILYEILLGKATLYAVLVSGLVLMAMVKKKNS | |
| ß chain w/alternative | MHCRLLSCVAFCLLGIGPLETAVFQTPNYHVTQVGNEVSFNCKQTLGHDTMY | 2670 |
| signal peptide, Cß | WYKQDSKKLLKIMESYNNKQLIVNETVPRRFSPQSSDKAHLNLRIKSVEPED | |
| (substituted) | SAVYLCASSSRDWSAETLYFGSGTRLTVLEDLRNVTPPKVSLFEPSKAEIAN | |
| | KQKATLVCLARGFFPDHVELSWWVNGKEVHSGVSTDPQAYKESNYSYCLSSR | |
| | LRVSATFWHNPRNHFRCQVQFHGLSEEDKWPEGSPKPVTQNISAEAWGRADC | |
| | GITSASYHQGVLSATILYEILLGKATLYAVLVSGLVLMAMVKKKNS | |

In some embodiments, TCR067 interacts with and/or is specific for KRAS. In some embodiments, the peptide is from a neoantigen of KRAS. In some embodiments, the neoantigen has the amino acid changes G12D and/or G12V relative to the wild type KRAS sequence. In some embodiments, TCR067 interacts with the neoantigen in the context of HLA-A11, as described in International Publication No. WO 2016/085904, incorporated herein by reference in its entirety.

TABLE 6BP
Amino acid sequences of TCR068.

| | | SEQ |
|---|---|---|
| Description | Sequence | ID NO: |
| CDR1α | DPNSYY | 1671 |
| CDR2α | VFSSTEI | 1672 |
| CDR3α | AVSGGTNSAGNKLT | 1673 |
| Vα w/o signal peptide | EQVEQRPPHLSVREGDSAVITCTYTDPNSYYFFWYKQEPGASLQLLMKVESS | 1674 |
| (SignalP) | TEINEGQGFTVLLNKKDKRLSLNLTAAHPGDSAAYFCAVSGGTNSAGNKLTF | |
| | GIGTRVLVRP | |
| Vα w/o signal peptide | GEQVEQRPPHLSVREGDSAVITCTYTDPNSYYFFWYKQEPGASLQLLMKVFS | 1675 |
| (IMGT) | STEINEGQGFTVLLNKKDKRLSLNLTAAHPGDSAAYFCAVSGGTNSAGNKLT | |
| | FGIGTRVLVRP | |
| Vα | MXTVTGPLFLCFWLQLNCVSRGEQVEQRPPHLSVREGDSAVITCTYTDPNSY | 1676 |
| | YFFWYKQEPGASLQLLMKVFSSTEINEGQGFTVLLNKKDKRLSLNLTAAHPG | 1677 |
| | DSAAYFCAVSGGTNSAGNKLTFGIGTRVLVRP | |
| | (X = any amino acid) | |
| α chain w/WT signal | MKTVTGPLFLCFWLQLNCVSRGEQVEQRPPHLSVREGDSAVITCTYTDPNSY | 1678 |
| peptide, Cα | YFFWYKQEPGASLQLLMKVFSSTEINEGQGFTVLLNKKDKRLSLNLTAAHPG | |
| (substituted) | DSAAYFCAVSGGTNSAGNKLTFGIGTRVLVRPDIQNPEPAVYQLKDPRSQDS | |
| | TLCLFTDFDSQINVPKTMESGTFITDKTVLDMKAMDSKSNGAIAWSNQTSFT | |
| | CQDIFKETNATYPSSDVPCDATLTEKSFETDMNLNFQNLSVMGLRILLLKVA | |
| | GFNLLMTLRLWSS | |
| α chain w/alternative | MATVTGPLFLCFWLQLNCVSRGEQVEQRPPHLSVREGDSAVITCTYTDPNSY | 1679 |
| signal peptide, Cα | YFFWYKQEPGASLQLLMKVFSSTEINEGQGFTVLLNKKDKRLSLNLTAAHPG | |
| (substituted) | DSAAYFCAVSGGTNSAGNKLTFGIGTRVLVRPDIQNPEPAVYQLKDPRSQDS | |
| | TLCLFTDFDSQINVPKTMESGTFITDKTVLDMKAMDSKSNGAIAWSNQTSFT | |
| | CQDIFKETNATYPSSDVPCDATLTEKSFETDMNLNFQNLSVMGLRILLLKVA | |
| | GFNLLMTLRLWSS | |
| α chain w/alternative | MHTVTGPLFLCFWLQLNCVSRGEQVEQRPPHLSVREGDSAVITCTYTDPNSY | 1680 |
| signal peptide, Cα | YFFWYKQEPGASLQLLMKVFSSTEINEGQGFTVLLNKKDKRLSLNLTAAHPG | |
| (substituted) | DSAAYFCAVSGGTNSAGNKLTFGIGTRVLVRPDIQNPEPAVYQLKDPRSQDS | |
| | TLCLFTDFDSQINVPKTMESGTFITDKTVLDMKAMDSKSNGAIAWSNQTSFT | |
| | CQDIFKETNATYPSSDVPCDATLTEKSFETDMNLNFQNLSVMGLRILLLKVA | |
| | GFNLLMTLRLWSS | |
| CDR1ß | LGHDT | 2671 |
| CDR2ß | YNNKQL | 2672 |
| CDR3ß | ASSRDWGPAEQF | 2673 |
| Vß w/o signal peptide | AVFQTPNYHVTQVGNEVSFNCKQTLGHDTMYWYKQDSKKLLKIMFSYNNKQL | 2674 |
| (SignalP) | IVNETVPRRFSPQSSDKAHLNLRIKSVEPEDSAVYLCASSRDWGPAEQFFGP | |
| | GTRLTVL | |
| Vß w/o signal peptide | ETAVFQTPNYHVTQVGNEVSFNCKQTLGHDTMYWYKQDSKKLLKIMFSYNNK | 2675 |
| (IMGT) | QLIVNETVPRRFSPQSSDKAHLNLRIKSVEPEDSAVYLCASSRDWGPAEQFF | |
| | GPGTRLTVL | |
| Vß | MXCRLLSCVAFCLLGIGPLETAVFQTPNYHVTQVGNEVSFNCKQTLGHDTMY | 2676 |
| | WYKQDSKKLLKIMFSYNNKQLIVNETVPRRFSPQSSDKAHLNLRIKSVEPED | 2677 |
| | SAVYLCASSRDWGPAEQFFGPGTRLTVL | |
| | (X = any amino acid) | |
| ß chain w/WT signal | MGCRLLSCVAFCLLGIGPLETAVFQTPNYHVTQVGNEVSFNCKQTLGHDTMY | 2678 |
| peptide, Cß | WYKQDSKKLLKIMFSYNNKQLIVNETVPRRFSPQSSDKAHLNLRIKSVEPED | |
| (substituted) | SAVYLCASSRDWGPAEQFFGPGTRLTVLEDLRNVTPPKVSLFEPSKAEIANK | |
| | QKATLVCLARGFFPDHVELSWWVNGKEVHSGVSTDPQAYKESNYSYCLSSRL | |
| | RVSATFWHNPRNHFRCQVQFHGLSEEDKWPEGSPKPVTQNISAEAWGRADCG | |
| | ITSASYHQGVLSATILYEILLGKATLYAVLVSGLVLMAMVKKKNS | |
| ß chain w/alternative | MACRLLSCVAFCLLGIGPLETAVFQTPNYHVTQVGNEVSFNCKQTLGHDTMY | 2679 |
| signal peptide, Cß | WYKQDSKKLLKIMFSYNNKQLIVNETVPRRFSPQSSDKAHLNLRIKSVEPED | |
| (substituted) | SAVYLCASSRDWGPAEQFFGPGTRLTVLEDLRNVTPPKVSLFEPSKAEIANK | |
| | QKATLVCLARGFFPDHVELSWWVNGKEVHSGVSTDPQAYKESNYSYCLSSRL | |
| | RVSATFWHNPRNHFRCQVQFHGLSEEDKWPEGSPKPVTQNISAEAWGRADCG | |
| | ITSASYHQGVLSATILYEILLGKATLYAVLVSGLVLMAMVKKKNS | |
| ß chain w/alternative | MHCRLLSCVAFCLLGIGPLETAVFQTPNYHVTQVGNEVSFNCKQTLGHDTMY | 2680 |
| signal peptide, Cß | WYKQDSKKLLKIMFSYNNKQLIVNETVPRRFSPQSSDKAHLNLRIKSVEPED | |
| (substituted) | SAVYLCASSRDWGPAEQFFGPGTRLTVLEDLRNVTPPKVSLFEPSKAEIANK | |
| | QKATLVCLARGFFPDHVELSWWVNGKEVHSGVSTDPQAYKESNYSYCLSSRL | |
| | RVSATFWHNPRNHFRCQVQFHGLSEEDKWPEGSPKPVTQNISAEAWGRADCG | |
| | ITSASYHQGVLSATILYEILLGKATLYAVLVSGLVLMAMVKKKNS | |

In some embodiments, TCR068 interacts with and/or is specific for KRAS. In some embodiments, the peptide is from a neoantigen of KRAS. In some embodiments, the neoantigen has the amino acid changes G12D and/or G12V relative to the wild type KRAS sequence. In some embodiments, TCR068 interacts with the neoantigen in the context of HLA-A11, as described in International Publication No. WO 2016/085904, incorporated herein by reference in its entirety.

TABLE 6BQ
Amino acid sequences of TCR069.

| | | SEQ |
|---|---|---|
| Description | Sequence | ID NO: |
| CDR1α | NDMFDY | 1681 |
| CDR2α | VRSNVDK | 1682 |
| CDR3α | AAGDSGGSNYKLT | 1683 |
| Vα w/o signal peptide | QQKTGGQQVKQSSPSLTVQEGGILILNCDYENDMFDYFAWYKKYPDNSPTLL | 1684 |
| (SignalP) | ISVRSNVDKREDGRFTVFLNKSGKHFSLHITASQPEDTAVYLCAAGDSGGSN | |
| | YKLTFGKGTLLTVTP | |
| Vα w/o signal peptide | QQKTGGQQVKQSSPSLTVQEGGILILNCDYENDMFDYFAWYKKYPDNSPTLL | 1685 |
| (IMGT) | ISVRSNVDKREDGRFTVFLNKSGKHFSLHITASQPEDTAVYLCAAGDSGGSN | |
| | YKLTFGKGTLLTVTP | |
| Vα | MXGFLKALLLVLCLRPEWIKSQQKTGGQQVKQSSPSLTVQEGGILILNCDYE | 1686 |
| | NDMFDYFAWYKKYPDNSPTLLISVRSNVDKREDGRFTVELNKSGKHFSLHIT | 1687 |
| | ASQPEDTAVYLCAAGDSGGSNYKLTFGKGTLLTVTP | |
| | (X = any amino acid) | |
| α chain w/WT signal | MTGFLKALLLVLCLRPEWIKSQQKTGGQQVKQSSPSLTVQEGGILILNCDYE | 1688 |
| peptide, Cα | NDMFDYFAWYKKYPDNSPTLLISVRSNVDKREDGRFTVFLNKSGKHFSLHIT | |
| (substituted) | ASQPEDTAVYLCAAGDSGGSNYKLTFGKGTLLTVTPNIQNPEPAVYQLKDPR | |
| | SQDSTLCLFTDFDSQINVPKTMESGTFITDKTVLDMKAMDSKSNGAIAWSNQ | |
| | TSFTCQDIFKETNATYPSSDVPCDATLTEKSFETDMNLNFQNLSVMGLRILL | |
| | LKVAGFNLLMTLRLWSS | |
| α chain w/alternative | MAGFLKALLLVLCLRPEWIKSQQKTGGQQVKQSSPSLTVQEGGILILNCDYE | 1689 |
| signal peptide, Cα | NDMFDYFAWYKKYPDNSPTLLISVRSNVDKREDGRFTVFLNKSGKHFSLHIT | |
| (substituted) | ASQPEDTAVYLCAAGDSGGSNYKLTFGKGTLLTVTPNIQNPEPAVYQLKDPR | |
| | SQDSTLCLFTDFDSQINVPKTMESGTFITDKTVLDMKAMDSKSNGAIAWSNQ | |
| | TSFTCQDIFKETNATYPSSDVPCDATLTEKSFETDMNLNFQNLSVMGLRILL | |
| | LKVAGFNLLMTLRLWSS | |
| α chain w/alternative | MHGFLKALLLVLCLRPEWIKSQQKTGGQQVKQSSPSLTVQEGGILILNCDYE | 1690 |
| signal peptide, Cα | NDMFDYFAWYKKYPDNSPTLLISVRSNVDKREDGRFTVFLNKSGKHFSLHIT | |
| (substituted) | ASQPEDTAVYLCAAGDSGGSNYKLTFGKGTLLTVTPNIQNPEPAVYQLKDPR | |
| | SQDSTLCLFTDFDSQINVPKTMESGTFITDKTVLDMKAMDSKSNGAIAWSNQ | |
| | TSFTCQDIFKETNATYPSSDVPCDATLTEKSFETDMNLNFQNLSVMGLRILL | |
| | LKVAGFNLLMTLRLWSS | |
| CDR1ß | NSHNY | 2681 |
| CDR2ß | SYGAGN | 2682 |
| CDR3ß | ASASWGGYAEQF | 2683 |
| Vß w/o signal peptide | AVTQSPRNKVTVTGGNVTLSCRQTNSHNYMYWYRQDTGHGLRLIHYSYGAGN | 2684 |
| (SignalP) | LQIGDVPDGYKATRTTQEDFFLLLELASPSQTSLYFCASASWGGYAEQFFGP | |
| | GTRLTVL | |
| Vß w/o signal peptide | EAAVTQSPRNKVTVTGGNVTLSCRQTNSHNYMYWYRQDTGHGLRLIHYSYGA | 2685 |
| (IMGT) | GNLQIGDVPDGYKATRTTQEDFFLLLELASPSQTSLYFCASASWGGYAEQFF | |
| | GPGTRLTVL | |
| Vß | MXSRLFLVLSLLCTKHMEAAVTQSPRNKVTVTGGNVTLSCRQTNSHNYMYWY | 2686 |
| | RQDTGHGLRLIHYSYGAGNLQIGDVPDGYKATRTTQEDFFLLLELASPSQTS | 2687 |
| | LYFCASASWGGYAEQFFGPGTRLTVL | |
| | (X = any amino acid) | |
| ß chain w/WT signal | MGSRLFLVLSLLCTKHMEAAVTQSPRNKVTVTGGNVTLSCRQTNSHNYMYWY | 2688 |
| peptide, Cß | RQDTGHGLRLIHYSYGAGNLQIGDVPDGYKATRTTQEDFFLLLELASPSQTS | |
| (substituted) | LYFCASASWGGYAEQFFGPGTRLTVLEDLRNVTPPKVSLFEPSKAEIANKQK | |
| | ATLVCLARGFFPDHVELSWWVNGKEVHSGVSTDPQAYKESNYSYCLSSRLRV | |
| | SATFWHNPRNHFRCQVQFHGLSEEDKWPEGSPKPVTQNISAEAWGRADCGIT | |
| | SASYHQGVLSATILYEILLGKATLYAVLVSGLVLMAMVKKKNS | |
| ß chain w/alternative | MASRLFLVLSLLCTKHMEAAVTQSPRNKVTVTGGNVTLSCRQTNSHNYMYWY | 2689 |
| signal peptide, Cß | RQDTGHGLRLIHYSYGAGNLQIGDVPDGYKATRTTQEDFFLLLELASPSQTS | |
| (substituted) | LYFCASASWGGYAEQFFGPGTRLTVLEDLRNVTPPKVSLFEPSKAEIANKQK | |
| | ATLVCLARGFFPDHVELSWWVNGKEVHSGVSTDPQAYKESNYSYCLSSRLRV | |
| | SATFWHNPRNHFRCQVQFHGLSEEDKWPEGSPKPVTQNISAEAWGRADCGIT | |
| | SASYHQGVLSATILYEILLGKATLYAVLVSGLVLMAMVKKKNS | |
| ß chain w/alternative | MHSRLFLVLSLLCTKHMEAAVTQSPRNKVTVTGGNVTLSCRQTNSHNYMYWY | 2690 |
| signal peptide, Cß | RQDTGHGLRLIHYSYGAGNLQIGDVPDGYKATRTTQEDFFLLLELASPSQTS | |
| (substituted) | LYFCASASWGGYAEQFFGPGTRLTVLEDLRNVTPPKVSLFEPSKAEIANKQK | |
| | ATLVCLARGFFPDHVELSWWVNGKEVHSGVSTDPQAYKESNYSYCLSSRLRV | |
| | SATFWHNPRNHFRCQVQFHGLSEEDKWPEGSPKPVTQNISAEAWGRADCGIT | |
| | SASYHQGVLSATILYEILLGKATLYAVLVSGLVLMAMVKKKNS | |

In some embodiments, TCR069 interacts with and/or is specific for KRAS. In some embodiments, the peptide is from a neoantigen of KRAS. In some embodiments, the neoantigen has the amino acid changes G12D and/or G12V relative to the wild type KRAS sequence. In some embodiments, TCR069 interacts with the neoantigen in the context of HLA-A11, as described in International Publication No. WO 2016/085904, incorporated herein by reference in its entirety.

TABLE 6BR
Amino acid sequences of TCR070.

| | | SEQ |
|---|---|---|
| Description | Sequence | ID NO: |
| CDR1α | TTMRS | 1691 |
| CDR2α | LASGT | 1692 |
| CDR3α | AADSSNTGYQNFY | 1693 |
| Vα w/o signal peptide | DQVEQSPSALSLHEGTDSALRCNFTTTMRSVQWFRQNSRGSLISLFYLASGT | 1694 |
| (SignalP) | KENGRLKSAFDSKERRYSTLHIRDAQLEDSGTYFCAADSSNTGYQNFYFGKG | |
| | TSLTVIP | |
| Vα w/o signal peptide | GDQVEQSPSALSLHEGTDSALRCNFTTTMRSVQWFRQNSRGSLISLFYLASG | 1695 |
| (IMGT) | TKENGRLKSAFDSKERRYSTLHIRDAQLEDSGTYFCAADSSNTGYQNFYFGK | |
| | GTSLTVIP | |
| Vα | MXRNLGAVLGILWVQICWVRGDQVEQSPSALSLHEGTDSALRCNFTTTMRSV | 1696 |
| | QWFRQNSRGSLISLFYLASGTKENGRLKSAFDSKERRYSTLHIRDAQLEDSG | 1697 |
| | TYFCAADSSNTGYQNFYFGKGTSLTVIP | |
| | (X = any amino acid) | |
| α chain w/WT signal | MQRNLGAVLGILWVQICWVRGDQVEQSPSALSLHEGTDSALRCNFTTTMRSV | 1698 |
| peptide, Cα | QWFRQNSRGSLISLFYLASGTKENGRLKSAFDSKERRYSTLHIRDAQLEDSG | |
| (substituted) | TYFCAADSSNTGYQNFYFGKGTSLTVIPNIQNPEPAVYQLKDPRSQDSTLCL | |
| | FTDFDSQINVPKTMESGTFITDKTVLDMKAMDSKSNGAIAWSNQTSFTCQDI | |
| | FKETNATYPSSDVPCDATLTEKSFETDMNLNFQNLSVMGLRILLLKVAGFNL | |
| | LMTLRLWSS | |
| α chain w/alternative | MARNLGAVLGILWVQICWVRGDQVEQSPSALSLHEGTDSALRCNFTTTMRSV | 1699 |
| signal peptide, Cα | QWFRQNSRGSLISLFYLASGTKENGRLKSAFDSKERRYSTLHIRDAQLEDSG | |
| (substituted) | TYFCAADSSNTGYQNFYFGKGTSLTVIPNIQNPEPAVYQLKDPRSQDSTLCL | |
| | FTDFDSQINVPKTMESGTFITDKTVLDMKAMDSKSNGAIAWSNQTSFTCQDI | |
| | FKETNATYPSSDVPCDATLTEKSFETDMNLNFQNLSVMGLRILLLKVAGFNL | |
| | LMTLRLWSS | |
| α chain w/alternative | MHRNLGAVLGILWVQICWVRGDQVEQSPSALSLHEGTDSALRCNFTTTMRSV | 1700 |
| signal peptide, Cα | QWFRQNSRGSLISLFYLASGTKENGRLKSAFDSKERRYSTLHIRDAQLEDSG | |
| (substituted) | TYFCAADSSNTGYQNFYFGKGTSLTVIPNIQNPEPAVYQLKDPRSQDSTLCL | |
| | FTDFDSQINVPKTMESGTFITDKTVLDMKAMDSKSNGAIAWSNQTSFTCQDI | |
| | FKETNATYPSSDVPCDATLTEKSFETDMNLNFQNLSVMGLRILLLKVAGFNL | |
| | LMTLRLWSS | |
| CDR1ß | SGHLS | 2691 |
| CDR2ß | HYDKME | 2692 |
| CDR3ß | ASSLTDPLDSDYT | 2693 |
| Vß w/o signal peptide | NSGVVQSPRYIIKGKGERSILKCIPISGHLSVAWYQQTQGQELKFFIQHYDK | 2694 |
| (SignalP) | MERDKGNLPSRFSVQQFDDYHSEMNMSALELEDSAVYFCASSLTDPLDSDYT | |
| | FGSGTRLLVI | |
| Vß w/o signal peptide | SGVVQSPRYIIKGKGERSILKCIPISGHLSVAWYQQTQGQELKFFIQHYDKM | 2695 |
| (IMGT) | ERDKGNLPSRFSVQQFDDYHSEMNMSALELEDSAVYFCASSLTDPLDSDYTF | |
| | GSGTRLLVI | |
| Vß | MXNTAFPDPAWNTTLLSWVALFLLGTSSANSGVVQSPRYIIKGKGERSILKC | 2696 |
| | IPISGHLSVAWYQQTQGQELKFFIQHYDKMERDKGNLPSRFSVQQFDDYHSE | 2697 |
| | MNMSALELEDSAVYFCASSLTDPLDSDYTFGSGTRLLVI | |
| | (X = any amino acid) | |
| ß chain w/WT signal | MSNTAFPDPAWNTTLLSWVALFLLGTSSANSGVVQSPRYIIKGKGERSILKC | 2698 |
| peptide, Cß | IPISGHLSVAWYQQTQGQELKFFIQHYDKMERDKGNLPSRFSVQQFDDYHSE | |
| (substituted) | MNMSALELEDSAVYFCASSLTDPLDSDYTFGSGTRLLVIEDLRNVTPPKVSL | |
| | FEPSKAEIANKQKATLVCLARGFFPDHVELSWWVNGKEVHSGVSTDPQAYKE | |
| | SNYSYCLSSRLRVSATFWHNPRNHFRCQVQFHGLSEEDKWPEGSPKPVTQNI | |
| | SAEAWGRADCGITSASYQQGVLSATILYEILLGKATLYAVLVSTLVVMAMVK | |
| | RKNS | |
| ß chain w/alternative | MANTAFPDPAWNTTLLSWVALFLLGTSSANSGVVQSPRYIIKGKGERSILKC | 2699 |
| signal peptide, Cß | IPISGHLSVAWYQQTQGQELKFFIQHYDKMERDKGNLPSRFSVQQEDDYHSE | |
| (substituted) | MNMSALELEDSAVYFCASSLTDPLDSDYTFGSGTRLLVIEDLRNVTPPKVSL | |
| | FEPSKAEIANKQKATLVCLARGFFPDHVELSWWVNGKEVHSGVSTDPQAYKE | |
| | SNYSYCLSSRLRVSATFWHNPRNHFRCQVQFHGLSEEDKWPEGSPKPVTQNI | |
| | SAEAWGRADCGITSASYQQGVLSATILYEILLGKATLYAVLVSTLVVMAMVK | |
| | RKNS | |
| ß chain w/alternative | MHNTAFPDPAWNTTLLSWVALFLLGTSSANSGVVQSPRYIIKGKGERSILKC | 2700 |
| signal peptide, Cß | IPISGHLSVAWYQQTQGQELKFFIQHYDKMERDKGNLPSRFSVQQFDDYHSE | |
| (substituted) | MNMSALELEDSAVYFCASSLTDPLDSDYTFGSGTRLLVIEDLRNVTPPKVSL | |
| | FEPSKAEIANKQKATLVCLARGFFPDHVELSWWVNGKEVHSGVSTDPQAYKE | |
| | SNYSYCLSSRLRVSATFWHNPRNHFRCQVQFHGLSEEDKWPEGSPKPVTQNI | |
| | SAEAWGRADCGITSASYQQGVLSATILYEILLGKATLYAVLVSTLVVMAMVK | |
| | RKNS | |

In some embodiments, TCR070 interacts with and/or is specific for KRAS. In some embodiments, the peptide is from a neoantigen of KRAS. In some embodiments, the neoantigen has the amino acid changes G12D and/or G12V relative to the wild type KRAS sequence. In some embodiments, TCR070 interacts with the neoantigen in the context of HLA-A11, as described in International Publication No. WO 2016/085904, incorporated herein by reference in its entirety.

TABLE 6BS
Amino acid sequences of TCR071.

| | | SEQ |
|---|---|---|
| Description | Sequence | ID NO: |
| CDR1α | TTMRS | 1701 |
| CDR2α | LASGT | 1702 |
| CDR3α | AADSSNTZYQNFY | 1703 |
| | (Z = alanine, arginine, asparagine, aspartic acid, cysteine, | |
| | glutamic acid, glutamine, histidine, isoleucine, leucine, | |
| | lysine, methionine, phenylalanine, proline, serine, threonine, | |
| | tryptophan, tyrosine, or valine) | |
| Vα w/o signal peptide | DQVEQSPSALSLHEGTDSALRCNFTTTMRSVQWFRQNSRGSLISLFYLASGT | 1704 |
| (SignalP) | KENGRLKSAFDSKERRYSTLHIRDAQLEDSGTYFCAADSSNTZYQNFYFGKG | |
| | TSLTVIP | |
| | (Z = alanine, arginine, asparagine, aspartic acid, cysteine, | |
| | glutamic acid, glutamine, histidine, isoleucine, leucine, | |
| | lysine, methionine, phenylalanine, proline, serine, threonine, | |
| | tryptophan, tyrosine, or valine) | |
| Vα w/o signal peptide | GDQVEQSPSALSLHEGTDSALRCNFTTTMRSVQWFRQNSRGSLISLFYLASG | 1705 |
| (IMGT) | TKENGRLKSAFDSKERRYSTLHIRDAQLEDSGTYFCAADSSNTZYQNFYFGK | |
| | GTSLTVIP | |
| | (Z = alanine, arginine, asparagine, aspartic acid, cysteine, | |
| | glutamic acid, glutamine, histidine, isoleucine, leucine, | |
| | lysine, methionine, phenylalanine, proline, serine, threonine, | |
| | tryptophan, tyrosine, or valine) | |
| Vα | MXRNLGAVLGILWVQICWVRGDQVEQSPSALSLHEGTDSALRCNFTTTMRSV | 1706 |
| | QWFRQNSRGSLISLFYLASGTKENGRLKSAFDSKERRYSTLHIRDAQLEDSG | |
| | TYFCAADSSNTZYQNFYFGKGTSLTVIP | |
| | (X = any amino acid) | |
| | (Z = alanine, arginine, asparagine, aspartic acid, cysteine, | 1707 |
| | glutamic acid, glutamine, histidine, isoleucine, leucine, | |
| | lysine, methionine, phenylalanine, proline, serine, threonine, | |
| | tryptophan, tyrosine, or valine) | |
| α chain w/WT signal | MQRNLGAVLGILWVQICWVRGDQVEQSPSALSLHEGTDSALRCNFTTTMRSV | 1708 |
| peptide, Cα | QWFRQNSRGSLISLFYLASGTKENGRLKSAFDSKERRYSTLHIRDAQLEDSG | |
| (substituted) | TYFCAADSSNTZYQNFYFGKGTSLTVIPNIQNPEPAVYQLKDPRSQDSTLCL | |
| | FTDFDSQINVPKTMESGTFITDKTVLDMKAMDSKSNGAIAWSNQTSFTCQDI | |
| | FKETNATYPSSDVPCDATLTEKSFETDMNLNFQNLSVMGLRILLLKVAGFNL | |
| | LMTLRLWSS | |
| | (Z = alanine, arginine, asparagine, aspartic acid, cysteine, | |
| | glutamic acid, glutamine, histidine, isoleucine, leucine, | |
| | lysine, methionine, phenylalanine, proline, serine, threonine, | |
| | tryptophan, tyrosine, or valine) | |
| α chain w/alternative | MARNLGAVLGILWVQICWVRGDQVEQSPSALSLHEGTDSALRCNFTTTMRSV | 1709 |
| signal peptide, Cα | QWFRQNSRGSLISLFYLASGTKENGRLKSAFDSKERRYSTLHIRDAQLEDSG | |
| (substituted) | TYFCAADSSNTZYQNFYFGKGTSLTVIPNIQNPEPAVYQLKDPRSQDSTLCL | |
| | FTDFDSQINVPKTMESGTFITDKTVLDMKAMDSKSNGAIAWSNQTSFTCQDI | |
| | FKETNATYPSSDVPCDATLTEKSFETDMNLNFQNLSVMGLRILLLKVAGFNL | |
| | LMTLRLWSS | |
| | (Z = alanine, arginine, asparagine, aspartic acid, cysteine, | |
| | glutamic acid, glutamine, histidine, isoleucine, leucine, | |
| | lysine, methionine, phenylalanine, proline, serine, threonine, | |
| | tryptophan, tyrosine, or valine) | |
| α chain w/alternative | MHRNLGAVLGILWVQICWVRGDQVEQSPSALSLHEGTDSALRCNFTTTMRSV | 1710 |
| signal peptide, Cα | QWFRQNSRGSLISLFYLASGTKENGRLKSAFDSKERRYSTLHIRDAQLEDSG | |
| (substituted) | TYFCAADSSNTZYQNFYFGKGTSLTVIPNIQNPEPAVYQLKDPRSQDSTLCL | |
| | FTDFDSQINVPKTMESGTFITDKTVLDMKAMDSKSNGAIAWSNQTSFTCQDI | |
| | FKETNATYPSSDVPCDATLTEKSFETDMNLNFQNLSVMGLRILLLKVAGFNL | |
| | LMTLRLWSS | |
| | (Z = alanine, arginine, asparagine, aspartic acid, cysteine, | |
| | glutamic acid, glutamine, histidine, isoleucine, leucine, | |
| | lysine, methionine, phenylalanine, proline, serine, threonine, | |
| | tryptophan, tyrosine, or valine) | |
| CDR1ß | SGHLS | 2701 |
| CDR2B | HYDKME | 2702 |
| CDR3ß | CASSLTDPLDSDYTF | 2703 |
| Vß w/o signal peptide | NSGVVQSPRYIIKGKGERSILKCIPISGHLSVAWYQQTQGQELKFFIQHYDK | 2704 |
| (SignalP) | MERDKGNLPSRFSVQQFDDYHSEMNMSALELEDSAVYFCASSLTDPLDSDYT | |
| | FGSGTRLLVI | |
| Vß w/o signal peptide | SGVVQSPRYIIKGKGERSILKCIPISGHLSVAWYQQTQGQELKFFIQHYDKM | 2705 |
| (IMGT) | ERDKGNLPSRFSVQQFDDYHSEMNMSALELEDSAVYFCASSLTDPLDSDYTF | |
| | GSGTRLLVI | |
| Vß | MXNTAFPDPAWNTTLLSWVALFLLGTSSANSGVVQSPRYIIKGKGERSILKC | 2706 |
| | IPISGHLSVAWYQQTQGQELKFFIQHYDKMERDKGNLPSRFSVQQFDDYHSE | 2707 |
| | MNMSALELEDSAVYFCASSLTDPLDSDYTFGSGTRLLVI | |
| | (X = any amino acid) | |
| ß chain w/WT signal | MSNTAFPDPAWNTTLLSWVALFLLGTSSANSGVVQSPRYIIKGKGERSILKC | 2708 |
| peptide, Cß | IPISGHLSVAWYQQTQGQELKFFIQHYDKMERDKGNLPSRFSVQQFDDYHSE | |
| (substituted) | MNMSALELEDSAVYFCASSLTDPLDSDYTFGSGTRLLVIEDLRNVTPPKVSL | |
| | FEPSKAEIANKQKATLVCLARGFFPDHVELSWWVNGKEVHSGVSTDPQAYKE | |
| | SNYSYCLSSRLRVSATFWHNPRNHFRCQVQFHGLSEEDKWPEGSPKPVTQNI | |
| | SAEAWGRADCGITSASYQQGVLSATILYEILLGKATLYAVLVSTLVVMAMVK | |
| | RKNS | |
| ß chain w/alternative | MANTAFPDPAWNTTLLSWVALFLLGTSSANSGVVQSPRYIIKGKGERSILKC | 2709 |
| signal peptide, Cß | IPISGHLSVAWYQQTQGQELKFFIQHYDKMERDKGNLPSRFSVQQFDDYHSE | 2710 |
| (substituted) | MNMSALELEDSAVYFCASSLTDPLDSDYTFGSGTRLLVIEDLRNVTPPKVSL | |
| | FEPSKAEIANKQKATLVCLARGFFPDHVELSWWVNGKEVHSGVSTDPQAYKE | |
| | SNYSYCLSSRLRVSATFWHNPRNHFRCQVQFHGLSEEDKWPEGSPKPVTQNI | |
| | SAEAWGRADCGITSASYQQGVLSATILYEILLGKATLYAVLVSTLVVMAMVK | |
| | RKNS | |

In some embodiments, TCR071 interacts with and/or is specific for KRAS. In some embodiments, the peptide is from a neoantigen of KRAS. In some embodiments, the neoantigen has the amino acid changes G12D and/or G12V relative to the wild type KRAS sequence. In some embodiments, TCR071 interacts with the neoantigen in the context of HLA-A11, as described in International Publication No. WO 2016/085904, incorporated herein by reference in its entirety.

TABLE 6BT
Amino acid sequences of TCR072.

| | | SEQ |
|---|---|---|
| Description | Sequence | ID NO: |
| CDR1α | DRGSQS | 1711 |
| CDR2α | IYSNGD | 1712 |
| CDR3α | AVEGAGSYQLT | 1713 |
| Vα w/o signal peptide | QQKEVEQNSGPLSVPEGAIASLNCTYSDRGSQSFFWYRQYSGKSPELIMFIY | 1714 |
| (SignalP) | SNGDKEDGRFTAQLNKASQYVSLLIRDSQPSDSATYLCAVEGAGSYQLTFGK | |
| | GTKLSVIP | |
| Vα w/o signal peptide | QKEVEQNSGPLSVPEGAIASLNCTYSDRGSQSFFWYRQYSGKSPELIMFIYS | 1715 |
| (IMGT) | NGDKEDGRFTAQLNKASQYVSLLIRDSQPSDSATYLCAVEGAGSYQLTFGKG | |
| | TKLSVIP | |
| Vα | MXSLRVLLVILWLQLSWVWSQQKEVEQNSGPLSVPEGAIASLNCTYSDRGSQ | 1716 |
| | SFFWYRQYSGKSPELIMFIYSNGDKEDGRFTAQLNKASQYVSLLIRDSQPSD | |
| | SATYLCAVEGAGSYQLTFGKGTKLSVIP | 1717 |
| | (X = any amino acid) | |
| α chain w/WT signal | MKSLRVLLVILWLQLSWVWSQQKEVEQNSGPLSVPEGAIASLNCTYSDRGSQ | 1718 |
| peptide, Cα | SFFWYRQYSGKSPELIMFIYSNGDKEDGRFTAQLNKASQYVSLLIRDSQPSD | |
| (substituted) | SATYLCAVEGAGSYQLTFGKGTKLSVIPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGFNL | |
| | LMTLRLWSS | |
| α chain w/alternative | MASLRVLLVILWLQLSWVWSQQKEVEQNSGPLSVPEGAIASLNCTYSDRGSQ | 1719 |
| signal peptide, Cα | SFFWYRQYSGKSPELIMFIYSNGDKEDGRFTAQLNKASQYVSLLIRDSQPSD | |
| (substituted) | SATYLCAVEGAGSYQLTFGKGTKLSVIPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGFNL | |
| | LMTLRLWSS | |
| α chain w/alternative | MHSLRVLLVILWLQLSWVWSQQKEVEQNSGPLSVPEGAIASLNCTYSDRGSQ | 1720 |
| signal peptide, Cα | SFFWYRQYSGKSPELIMFIYSNGDKEDGRFTAQLNKASQYVSLLIRDSQPSD | |
| (substituted) | SATYLCAVEGAGSYQLTFGKGTKLSVIPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGFNL | |
| | LMTLRLWSS | |
| CDR1ß | DFQATT | 2711 |
| CDR2ß | SNEGSKA | 2712 |
| CDR3ß | SASNRLAVNEQF | 2713 |
| Vß w/o signal peptide | GSGLGAVVSQHPSWVICKSGTSVKIECRSLDFQATTMFWYRQFPKQSLMLMA | 2714 |
| (SignalP) | TSNEGSKATYEQGVEKDKFLINHASLTLSTLTVTSAHPEDSSFYICSASNRL | |
| | AVNEQFFGPGTRLTVL | |
| Vß w/o signal peptide | GAVVSQHPSWVICKSGTSVKIECRSLDFQATTMFWYRQFPKQSLMLMATSNE | 2715 |
| (IMGT) | GSKATYEQGVEKDKFLINHASLTLSTLTVTSAHPEDSSFYICSASNRLAVNE | |
| | QFFGPGTRLTVL | |
| Vß | MXLLLLLLGPGISLLLPGSLAGSGLGAVVSQHPSWVICKSGTSVKIECRSLD | 2716 |
| | FQATTMFWYRQFPKQSLMLMATSNEGSKATYEQGVEKDKFLINHASLTLSTL | |
| | TVTSAHPEDSSFYICSASNRLAVNEQFFGPGTRLTVL | 2717 |
| | (X = any amino acid) | |
| ß chain w/WT signal | MLLLLLLLGPGISLLLPGSLAGSGLGAVVSQHPSWVICKSGTSVKIECRSLD | 2718 |
| peptide, Cß | FQATTMFWYRQFPKQSLMLMATSNEGSKATYEQGVEKDKFLINHASLTLSTL | |
| (substituted) | TVTSAHPEDSSFYICSASNRLAVNEQFFGPGTRLTVLEDLRNVTPPKVSLFE | |
| | PSKAEIANKQKATLVCLARGFFPDHVELSWWVNGKEVHSGVCTDPQAYKESN | |
| | YSYCLSSRLRVSATFWHNPRNHFRCQVQFHGLSEEDKWPEGSPKPVTQNISA | |
| | EAWGRADCGITSASYQQGVLSATILYEILLGKATLYAVLVSTLVVMAMVKRK | |
| | NS | |
| ß chain w/alternative | MALLLLLLGPGISLLLPGSLAGSGLGAVVSQHPSWVICKSGTSVKIECRSLD | 2719 |
| signal peptide, Cß | FQATTMFWYRQFPKQSLMLMATSNEGSKATYEQGVEKDKFLINHASLTLSTL | |
| (substituted) | TVTSAHPEDSSFYICSASNRLAVNEQFFGPGTRLTVLEDLRNVTPPKVSLFE | |
| | PSKAEIANKQKATLVCLARGFFPDHVELSWWVNGKEVHSGVCTDPQAYKESN | |
| | YSYCLSSRLRVSATFWHNPRNHFRCQVQFHGLSEEDKWPEGSPKPVTQNISA | |
| | EAWGRADCGITSASYQQGVLSATILYEILLGKATLYAVLVSTLVVMAMVKRK | |
| | NS | |
| ß chain w/alternative | MHLLLLLLGPGISLLLPGSLAGSGLGAVVSQHPSWVICKSGTSVKIECRSLD | 2720 |
| signal peptide, Cß | FQATTMFWYRQFPKQSLMLMATSNEGSKATYEQGVEKDKFLINHASLTLSTL | |
| (substituted) | TVTSAHPEDSSFYICSASNRLAVNEQFFGPGTRLTVLEDLRNVTPPKVSLFE | |
| | PSKAEIANKQKATLVCLARGFFPDHVELSWWVNGKEVHSGVCTDPQAYKESN | |
| | YSYCLSSRLRVSATFWHNPRNHFRCQVQFHGLSEEDKWPEGSPKPVTQNISA | |
| | EAWGRADCGITSASYQQGVLSATILYEILLGKATLYAVLVSTLVVMAMVKRK | |
| | NS | |

In some embodiments, TCR072 interacts with and/or is specific for KRAS. In some embodiments, the peptide is from a neoantigen of KRAS. In some embodiments, the neoantigen has the amino acid change G12R relative to the wild type KRAS sequence. In some embodiments, TCR072 interacts with the neoantigen in the context of HLA-DQA1*05:05:HLA-DQB1*03:01 heterodimer as described in International Publication No. WO 2020/154275, incorporated herein by reference in its entirety.

TABLE 6BU
Amino acid sequences of TCR073.

| | | SEQ |
|---|---|---|
| Description | Sequence | ID NO: |
| CDR1α | NSMFDY | 1721 |
| CDR2α | ISISSIKDK | 1722 |
| CDR3α | AASGNTGTASKLT | 1723 |
| Vα w/o signal peptide | QQKNDDQQVKQNSPSLSVQEGRISILNCDYTNSMFDYFLWYKKYPAEGPTFL | 1724 |
| (SignalP) | ISISSIKDKNEDGRFTVFLNKSAKHLSLHIVPSQPGDSAVYFCAASGNTGTA | |
| | SKLTFGTGTRLQVTL | |
| Vα w/o signal peptide | DQQVKQNSPSLSVQEGRISILNCDYTNSMFDYFLWYKKYPAEGPTFLISISS | 1725 |
| (IMGT) | IKDKNEDGRFTVFLNKSAKHLSLHIVPSQPGDSAVYFCAASGNTGTASKLTF | |
| | GTGTRLQVTL | |
| Vα | MXMLLGASVLILWLQPDWVNSQQKNDDQQVKQNSPSLSVQEGRISILNCDYT | 1726 |
| | NSMFDYFLWYKKYPAEGPTFLISISSIKDKNEDGRFTVFLNKSAKHLSLHIV | |
| | PSQPGDSAVYFCAASGNTGTASKLTFGTGTRLQVTL | 1727 |
| | (X = any amino acid) | |
| α chain w/WT signal | MAMLLGASVLILWLQPDWVNSQQKNDDQQVKQNSPSLSVQEGRISILNCDYT | 1728 |
| peptide, Cα | NSMFDYFLWYKKYPAEGPTFLISISSIKDKNEDGRFTVFLNKSAKHLSLHIV | |
| (substituted) | PSQPGDSAVYFCAASGNTGTASKLTFGTGTRLQVTLNIQNPEPAVYQLKDPR | |
| | SQDSTLCLFTDFDSQINVPKTMESGTFITDKCVLDMKAMDSKSNGAIAWSNQ | |
| | TSFTCQDIFKETNATYPSSDVPCDATLTEKSFETDMNLNFQNLLVIVLRILL | |
| | LKVAGFNLLMTLRLWSS | |
| α chain w/alternative | MHMLLGASVLILWLQPDWVNSQQKNDDQQVKQNSPSLSVQEGRISILNCDYT | 1729 |
| signal peptide, Cα | NSMFDYFLWYKKYPAEGPTFLISISSIKDKNEDGRFTVFLNKSAKHLSLHIV | 1730 |
| (substituted) | PSQPGDSAVYFCAASGNTGTASKLTFGTGTRLQVTLNIQNPEPAVYQLKDPR | |
| | SQDSTLCLFTDFDSQINVPKTMESGTFITDKCVLDMKAMDSKSNGAIAWSNQ | |
| | TSFTCQDIFKETNATYPSSDVPCDATLTEKSFETDMNLNFQNLLVIVLRILL | |
| | LKVAGFNLLMTLRLWSS | |
| CDR1ß | LNHDA | 2721 |
| CDR2ß | SQIVND | 2722 |
| CDR3ß | ASSNQLVTGGYGYT | 2723 |
| Vß w/o signal peptide | GITQSPKYLFRKEGQNVTLSCEQNLNHDAMYWYRQDPGQGLRLIYYSQIVND | 2724 |
| (SignalP) | FQKGDIAEGYSVSREKKESFPLTVTSAQKNPTAFYLCASSNQLVTGGYGYTE | |
| | GSGTRLTVV | |
| Vß w/o signal peptide | DGGITQSPKYLFRKEGQNVTLSCEQNLNHDAMYWYRQDPGQGLRLIYYSQIV | 2725 |
| (IMGT) | NDFQKGDIAEGYSVSREKKESFPLTVTSAQKNPTAFYLCASSNQLVTGGYGY | |
| | TFGSGTRLTVV | |
| Vß | MXNQVLCCVVLCFLGANTVDGGITQSPKYLFRKEGQNVTLSCEQNLNHDAMY | 2726 |
| | WYRQDPGQGLRLIYYSQIVNDFQKGDIAEGYSVSREKKESFPLTVTSAQKNP | 2727 |
| | TAFYLCASSNQLVTGGYGYTFGSGTRLTVV | |
| | (X = any amino acid) | |
| ß chain w/WT signal | MSNQVLCCVVLCFLGANTVDGGITQSPKYLFRKEGQNVTLSCEQNLNHDAMY | 2728 |
| peptide, Cß | WYRQDPGQGLRLIYYSQIVNDFQKGDIAEGYSVSREKKESFPLTVTSAQKNP | |
| (substituted) | TAFYLCASSNQLVTGGYGYTFGSGTRLTVVEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain w/alternative | MANQVLCCVVLCFLGANTVDGGITQSPKYLFRKEGQNVTLSCEQNLNHDAMY | 2729 |
| signal peptide, Cß | WYRQDPGQGLRLIYYSQIVNDFQKGDIAEGYSVSREKKESFPLTVTSAQKNP | |
| (substituted) | TAFYLCASSNQLVTGGYGYTFGSGTRLTVVEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain w/alternative | MHNQVLCCVVLCFLGANTVDGGITQSPKYLFRKEGQNVTLSCEQNINHDAMY | 2730 |
| signal peptide, Cß | WYRQDPGQGLRLIYYSQIVNDFQKGDIAEGYSVSREKKESFPLTVTSAQKNP | |
| (substituted) | TAFYLCASSNQLVTGGYGYTFGSGTRLTVVEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR073 interacts with and/or is specific for KRAS. In some embodiments, the peptide is from a neoantigen of KRAS. In some embodiments, the neoantigen has the amino acid change G12R relative to the wild type KRAS sequence. In some embodiments, TCR073 interacts with the neoantigen in the context of HLA-DRB5*01:HLA-DRA*01:01 heterodimer as described in International Publication No. WO 2020/154275, incorporated herein by reference in its entirety.

TABLE 6BV
Amino acid sequences of TCR074.

| | | SEQ |
|---|---|---|
| Description | Sequence | ID NO: |
| CDR1α | TIYSNAF | 1731 |
| CDR2α | SSTDNKR | 1732 |
| CDR3α | ALSEGGNYKYV | 1733 |
| Vα w/o signal peptide | DGDSVTQKEGLVTLTEGLPVMLNCTYQTIYSNAFLFWYVHYLNESPRLLLKS | 1734 |
| (SignalP) | STDNKRTEHQGFHATLHKSSSSFHLQKSSAQLSDSALYYCALSEGGNYKYVF | |
| | GAGTRLKVIA | |
| Vα w/o signal peptide | GDSVTQKEGLVTLTEGLPVMLNCTYQTIYSNAFLFWYVHYLNESPRLLLKSS | 1735 |
| (IMGT) | TDNKRTEHQGFHATLHKSSSSFHLQKSSAQLSDSALYYCALSEGGNYKYVFG | |
| | AGTRLKVIA | |
| Vα | MXPGTCSVLVLLLMLRRSNGDGDSVTQKEGLVTLTEGLPVMLNCTYQTIYSN | 1736 |
| | AFLFWYVHYLNESPRLLLKSSTDNKRTEHQGFHATLHKSSSSFHLQKSSAQL | 1737 |
| | SDSALYYCALSEGGNYKYVFGAGTRLKVIA | |
| | (X = any amino acid) | |
| α chain w/WT signal | MRPGTCSVLVLLLMLRRSNGDGDSVTQKEGLVTLTEGLPVMLNCTYQTIYSN | 1738 |
| peptide, Cα | AFLFWYVHYLNESPRLLLKSSTDNKRTEHQGFHATLHKSSSSFHLQKSSAQL | |
| (substituted) | SDSALYYCALSEGGNYKYVFGAGTRLKVIANIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGE | |
| | NLLMTLRLWSS | |
| α chain w/alternative | MAPGTCSVLVLLLMLRRSNGDGDSVTQKEGLVTLTEGLPVMLNCTYQTIYSN | 1739 |
| signal peptide, Cα | AFLFWYVHYLNESPRLLLKSSTDNKRTEHQGFHATLHKSSSSFHLQKSSAQL | |
| (substituted) | SDSALYYCALSEGGNYKYVFGAGTRLKVIANIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| α chain w/alternative | MHPGTCSVLVLLLMLRRSNGDGDSVTQKEGLVTLTEGLPVMLNCTYQTIYSN | 1740 |
| signal peptide, Cα | AFLFWYVHYLNESPRLLLKSSTDNKRTEHQGFHATLHKSSSSFHLQKSSAQL | |
| (substituted) | SDSALYYCALSEGGNYKYVFGAGTRLKVIANIQNPEPAVYQLKDPRSQDSTL | |
| | CLFTDFDSQINVPKTMESGTFITDKCVLDMKAMDSKSNGAIAWSNQTSFTCQ | |
| | DIFKETNATYPSSDVPCDATLTEKSFETDMNLNFQNLLVIVLRILLLKVAGF | |
| | NLLMTLRLWSS | |
| CDR1ß | NSQYPW | 2731 |
| CDR2ß | LRSPGD | 2732 |
| CDR3ß | TCSARHSAETLY | 2733 |
| Vß w/o signal peptide | DPTVTLLEQNPRWRLVPRGQAVNLRCILKNSQYPWMSWYQQDLQKQLQWLFT | 2734 |
| (SignalP) | LRSPGDKEVKSLPGADYLATRVTDTELRLQVANMSQGRTLYCTCSARHSAET | |
| | LYFGSGTRLTVL | |
| Vß w/o signal peptide | VTLLEQNPRWRLVPRGQAVNLRCILKNSQYPWMSWYQQDLQKQLQWLFTLRS | 2735 |
| (IMGT) | PGDKEVKSLPGADYLATRVTDTELRLQVANMSQGRTLYCTCSARHSAETLYF | |
| | GSGTRLTVL | |
| Vß | MXQFCILCLCVLMASVATDPTVTLLEQNPRWRLVPRGQAVNLRCILKNSQYP | 2736 |
| | WMSWYQQDLQKQLQWLFTLRSPGDKEVKSLPGADYLATRVTDTELRLQVANM | 2737 |
| | SQGRTLYCTCSARHSAETLYFGSGTRLTVL | |
| | (X = any amino acid) | |
| ß chain w/WT signal | MWQFCILCLCVLMASVATDPTVTLLEQNPRWRLVPRGQAVNLRCILKNSQYP | 2738 |
| peptide, Cß | WMSWYQQDLQKQLQWLFTLRSPGDKEVKSLPGADYLATRVTDTELRLQVANM | |
| (substituted) | SQGRTLYCTCSARHSAETLYFGSGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain w/alternative | MAQFCILCLCVLMASVATDPTVTLLEQNPRWRLVPRGQAVNLRCILKNSQYP | 2739 |
| signal peptide, Cß | WMSWYQQDLQKQLQWLFTLRSPGDKEVKSLPGADYLATRVTDTELRLQVANM | |
| (substituted) | SQGRTLYCTCSARHSAETLYFGSGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| ß chain w/alternative | MHQFCILCLCVLMASVATDPTVTLLEQNPRWRLVPRGQAVNLRCILKNSQYP | 2740 |
| signal peptide, Cß | WMSWYQQDLQKQLQWLFTLRSPGDKEVKSLPGADYLATRVTDTELRLQVANM | |
| (substituted) | SQGRTLYCTCSARHSAETLYFGSGTRLTVLEDLRNVTPPKVSLFEPSKAEIA | |
| | NKQKATLVCLARGFFPDHVELSWWVNGKEVHSGVCTDPQAYKESNYSYCLSS | |
| | RLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |

In some embodiments, TCR074 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR074 interacts with the neoantigen in the context of HLA-A3 heterodimer as described in International Publication No. WO 2020/086827, incorporated herein by reference in its entirety.

TABLE 6BW
Amino acid sequences of TCR075.

| | | SEQ |
|---|---|---|
| Description | Sequence | ID NO: |
| CDR1α | TSDPSYG | 1741 |
| CDR2α | QGSYDQQN | 1742 |
| CDR3α | AMRGASQGGSEKLV | 1743 |
| Vα w/o signal peptide | QKITQTQPGMFVQEKEAVTLDCTYDTSDPSYGLFWYKQPSSGEMIFLIYQGS | 1744 |
| (SignalP) | YDQQNATEGRYSLNFQKARKSANLVISASQLGDSAMYFCAMRGASQGGSEKL | |
| | VFGKGTKLTVNP | |
| Vα w/o signal peptide | AQKITQTQPGMFVQEKEAVTLDCTYDTSDPSYGLFWYKQPSSGEMIFLIYQG | 1745 |
| (IMGT) | SYDQQNATEGRYSLNFQKARKSANLVISASQLGDSAMYFCAMRGASQGGSEK | |
| | LVFGKGTKLTVNP | |
| Vα | MXLSSLLKVVTASLWLGPGIAQKITQTQPGMFVQEKEAVTLDCTYDTSDPSY | 1746 |
| | GLFWYKQPSSGEMIFLIYQGSYDQQNATEGRYSLNFQKARKSANLVISASQL | |
| | GDSAMYFCAMRGASQGGSEKLVFGKGTKLTVNP | 1747 |
| | (X = any amino acid) | |
| α chain w/WT signal | MSLSSLLKVVTASLWLGPGIAQKITQTQPGMFVQEKEAVTLDCTYDTSDPSY | 1748 |
| peptide, Cα | GLFWYKQPSSGEMIFLIYQGSYDQQNATEGRYSLNFQKARKSANLVISASQL | |
| (substituted) | GDSAMYFCAMRGASQGGSEKLVFGKGTKLTVNPNIQNPEPAVYQLKDPRSQD | |
| | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| α chain w/alternative | MALSSLLKVVTASLWLGPGIAQKITQTQPGMFVQEKEAVTLDCTYDTSDPSY | 1749 |
| signal peptide, Cα | GLFWYKQPSSGEMIFLIYQGSYDQQNATEGRYSLNFQKARKSANLVISASQL | |
| (substituted) | GDSAMYFCAMRGASQGGSEKLVFGKGTKLTVNPNIQNPEPAVYQLKDPRSQD | |
| | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| α chain w/alternative | MHLSSLLKVVTASLWLGPGIAQKITQTQPGMFVQEKEAVTLDCTYDTSDPSY | 1750 |
| signal peptide, Cα | GLFWYKQPSSGEMIFLIYQGSYDQQNATEGRYSLNFQKARKSANLVISASQL | |
| (substituted) | GDSAMYFCAMRGASQGGSEKLVFGKGTKLTVNPNIQNPEPAVYQLKDPRSQD | |
| | STLCLFTDFDSQINVPKTMESGTFITDKCVLDMKAMDSKSNGAIAWSNQTSF | |
| | TCQDIFKETNATYPSSDVPCDATLTEKSFETDMNLNFQNLLVIVLRILLLKV | |
| | AGFNLLMTLRLWSS | |
| CDR1ß | SGHRS | 2741 |
| CDR2ß | YFSETQ | 2742 |
| CDR3ß | ASSLTSGGFDEQF | 2743 |
| Vß w/o signal peptide | GVTQTPRYLIKTRGQQVTLSCSPISGHRSVSWYQQTPGQGLQFLFEYFSETQ | 2744 |
| (SignalP) | RNKGNFPGRFSGRQFSNSRSEMNVSTLELGDSALYLCASSLTSGGFDEQFFG | |
| | PGTRLTVL | |
| Vß w/o signal peptide | KAGVTQTPRYLIKTRGQQVTLSCSPISGHRSVSWYQQTPGQGLQFLFEYFSE | 2745 |
| (IMGT) | TQRNKGNFPGRFSGRQFSNSRSEMNVSTLELGDSALYLCASSLTSGGFDEQF | |
| | FGPGTRLTVL | |
| Vß | MXSRLLCWVLLCLLGAGPVKAGVTQTPRYLIKTRGQQVTLSCSPISGHRSVS | 2746 |
| | WYQQTPGQGLQFLFEYFSETQRNKGNFPGRESGRQFSNSRSEMNVSTLELGD | |
| | SALYLCASSLTSGGFDEQFFGPGTRLTVL | 2747 |
| | (X = any amino acid) | |
| ß chain w/WT signal | MGSRLLCWVLLCLLGAGPVKAGVTQTPRYLIKTRGQQVTLSCSPISGHRSVS | 2748 |
| peptide, Cß | WYQQTPGQGLQFLFEYFSETQRNKGNFPGRESGRQFSNSRSEMNVSTLELGD | |
| (substituted) | SALYLCASSLTSGGFDEQFFGPGTRLTVLEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| ß chain w/alternative | MASRLLCWVLLCLLGAGPVKAGVTQTPRYLIKTRGQQVTLSCSPISGHRSVS | 2749 |
| signal peptide, Cß | WYQQTPGQGLQFLFEYFSETQRNKGNFPGRFSGRQFSNSRSEMNVSTLELGD | |
| (substituted) | SALYLCASSLTSGGFDEQFFGPGTRLTVLEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |
| ß chain w/alternative | MHSRLLCWVLLCLLGAGPVKAGVTQTPRYLIKTRGQQVTLSCSPISGHRSVS | 2750 |
| signal peptide, Cß | WYQQTPGQGLQFLFEYFSETQRNKGNFPGRFSGRQFSNSRSEMNVSTLELGD | |
| (substituted) | SALYLCASSLTSGGFDEQFFGPGTRLTVLEDLRNVTPPKVSLFEPSKAEIAN | |
| | KQKATLVCLARGFFPDHVELSWWVNGKEVHSGVCTDPQAYKESNYSYCLSSR | |
| | LRVSATFWHNPRNHFRCQVQFHGLSEEDKWPEGSPKPVTQNISAEAWGRADC | |
| | GITSASYQQGVLSATILYEILLGKATLYAVLVSTLVVMAMVKRKNS | |

In some embodiments, TCR075 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR075 interacts with the neoantigen in the context of HLA-A*11:01, as described in International Publication No. WO 2019/112941, incorporated herein b reference in its entirety.

TABLE 6BX
Amino acid sequences of TCR076.

| | | SEQ |
|---|---|---|
| Description | Sequence | ID NO: |
| CDR1α | DSASNY | 1751 |
| CDR2α | IRSNVGE | 1752 |
| CDR3α | AASTGGGNKLT | 1753 |
| Vα w/o signal peptide | ENVEQHPSTLSVQEGDSAVIKCTYSDSASNYFPWYKQELGKGPQLIIDIRSN | 1754 |
| (SignalP) | VGEKKDQRIAVTLNKTAKHFSLHITETQPEDSAVYFCAASTGGGNKLTFGTG | |
| | TQLKVEL | |
| Vα w/o signal peptide | GFNVEQHPSTLSVQEGDSAVIKCTYSDSASNYFPWYKQELGKGPQLIIDIRS | 1755 |
| (IMGT) | NVGEKKDQRIAVTLNKTAKHFSLHITETQPEDSAVYFCAASTGGGNKLTFGT | |
| | GTQLKVEL | |
| Vα | MXSIRAVFIFLWLQLDLVNGFNVEQHPSTLSVQEGDSAVIKCTYSDSASNYF | 1756 |
| | PWYKQELGKGPQLIIDIRSNVGEKKDQRIAVTLNKTAKHFSLHITETQPEDS | 1757 |
| | AVYFCAASTGGGNKLTFGTGTQLKVEL | |
| | (X = any amino acid) | |
| α chain w/WT signal | MTSIRAVFIFLWLQLDLVNGFNVEQHPSTLSVQEGDSAVIKCTYSDSASNYF | 1758 |
| peptide, Cα | PWYKQELGKGPQLIIDIRSNVGEKKDQRIAVTLNKTAKHFSLHITETQPEDS | |
| (substituted) | AVYFCAASTGGGNKLTFGTGTQLKVELNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain w/alternative | MASIRAVFIFLWLQLDLVNGFNVEQHPSTLSVQEGDSAVIKCTYSDSASNYF | 1759 |
| signal peptide, Cα | PWYKQELGKGPQLIIDIRSNVGEKKDQRIAVTLNKTAKHFSLHITETQPEDS | |
| (substituted) | AVYFCAASTGGGNKLTFGTGTQLKVELNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain w/alternative | MHSIRAVFIFLWLQLDLVNGFNVEQHPSTLSVQEGDSAVIKCTYSDSASNYF | 1760 |
| signal peptide, Cα | PWYKQELGKGPQLIIDIRSNVGEKKDQRIAVTLNKTAKHFSLHITETQPEDS | |
| (substituted) | AVYFCAASTGGGNKLTFGTGTQLKVELNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| CDR1ß | DFQATT | 2751 |
| CDR2B | SNEGSKA | 2752 |
| CDR3ß | SAREGAGGMGTQY | 2753 |
| Vß w/o signal peptide | AVVSQHPSRVICKSGTSVKIECRSLDFQATTMFWYRQFPKQSLMLMATSNEG | 2754 |
| (SignalP) | SKATYEQGVEKDKFLINHASLTLSTLTVTSAHPEDSSFYICSAREGAGGMGT | |
| | QYFGPGTRLLVL | |
| Vß w/o signal peptide | GAVVSQHPSRVICKSGTSVKIECRSLDFQATTMFWYRQFPKQSLMLMATSNE | 2755 |
| (IMGT) | GSKATYEQGVEKDKFLINHASLTLSTLTVTSAHPEDSSFYICSAREGAGGMG | |
| | TQYFGPGTRLLVL | |
| Vß | MXLLLLLLGPAGSGLGAVVSQHPSRVICKSGTSVKIECRSLDFQATTMFWYR | 2756 |
| | QFPKQSLMLMATSNEGSKATYEQGVEKDKFLINHASLTLSTLTVTSAHPEDS | 2757 |
| | SFYICSAREGAGGMGTQYFGPGTRLLVL | |
| | (X = any amino acid) | |
| ß chain w/WT signal | MLLLLLLLGPAGSGLGAVVSQHPSRVICKSGTSVKIECRSLDFQATTMFWYR | 2758 |
| peptide, Cß | QFPKQSLMLMATSNEGSKATYEQGVEKDKFLINHASLTLSTLTVTSAHPEDS | |
| (substituted) | SFYICSAREGAGGMGTQYFGPGTRLLVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| ß chain w/alternative | MALLLLLLGPAGSGLGAVVSQHPSRVICKSGTSVKIECRSLDFQATTMFWYR | 2759 |
| signal peptide, Cß | QFPKQSLMLMATSNEGSKATYEQGVEKDKFLINHASLTLSTLTVTSAHPEDS | |
| (substituted) | SFYICSAREGAGGMGTQYFGPGTRLLVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |
| ß chain w/alternative | MHLLLLLLGPAGSGLGAVVSQHPSRVICKSGTSVKIECRSLDFQATTMFWYR | 2760 |
| signal peptide, Cß | QFPKQSLMLMATSNEGSKATYEQGVEKDKFLINHASLTLSTLTVTSAHPEDS | |
| (substituted) | SFYICSAREGAGGMGTQYFGPGTRLLVLEDLRNVTPPKVSLFEPSKAEIANK | |
| | QKATLVCLARGFFPDHVELSWWVNGKEVHSGVCTDPQAYKESNYSYCLSSRL | |
| | RVSATFWHNPRNHFRCQVQFHGLSEEDKWPEGSPKPVTQNISAEAWGRADCG | |
| | ITSASYQQGVLSATILYEILLGKATLYAVLVSTLVVMAMVKRKNS | |

In some embodiments, TCR076 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR076 interacts with the neoantigen in the context of HLA-DRB1*07:01, as described in International Publication No. WO 2019/060349, incorporated herein by reference in its entirety.

TABLE 6BY
Amino acid sequences of TCR077.

| | | SEQ |
|---|---|---|
| Description | Sequence | ID NO: |
| CDR1α | SSVPPY | 1761 |
| CDR2α | YTSAATLV | 1762 |
| CDR3α | AVSEDSNYQLI | 1763 |
| Vα w/o signal peptide | QSVTQLGSHVSVSEGALVLLRCNYSSSVPPYLFWYVQYPNQGLQLLLKYTSA | 1764 |
| (SignalP) | ATLVKGINGFEAEFKKSETSFHLTKPSAHMSDAAEYFCAVSEDSNYQLIWGA | |
| | GTKLIIKP | |
| Vα w/o signal peptide | AQSVTQLGSHVSVSEGALVLLRCNYSSSVPPYLFWYVQYPNQGLQLLLKYTS | 1765 |
| (IMGT) | AATLVKGINGFEAEFKKSETSFHLTKPSAHMSDAAEYFCAVSEDSNYQLIWG | |
| | AGTKLIIKP | |
| Vα | MXLLLVPVLEVIFTLGGTRAQSVTQLGSHVSVSEGALVLLRCNYSSSVPPYL | 1766 |
| | FWYVQYPNQGLQLLLKYTSAATLVKGINGFEAEFKKSETSFHLTKPSAHMSD | |
| | AAEYFCAVSEDSNYQLIWGAGTKLIIKP | 1767 |
| | (X = any amino acid) | |
| α chain w/WT signal | MLLLLVPVLEVIFTLGGTRAQSVTQLGSHVSVSEGALVLLRCNYSSSVPPYL | 1768 |
| peptide, Cα | FWYVQYPNQGLQLLLKYTSAATLVKGINGFEAEFKKSETSFHLTKPSAHMSD | |
| (substituted) | AAEYFCAVSEDSNYQLIWGAGTKLIIKPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGFNL | |
| | LMTLRLWSS | |
| α chain w/alternative | MALLLVPVLEVIFTLGGTRAQSVTQLGSHVSVSEGALVLLRCNYSSSVPPYL | 1769 |
| signal peptide, Cα | FWYVQYPNQGLQLLLKYTSAATLVKGINGFEAEFKKSETSFHLTKPSAHMSD | |
| (substituted) | AAEYFCAVSEDSNYQLIWGAGTKLIIKPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGFNL | |
| | LMTLRLWSS | |
| α chain w/alternative | MHLLLVPVLEVIFTLGGTRAQSVTQLGSHVSVSEGALVLLRCNYSSSVPPYL | 1770 |
| signal peptide, Cα | FWYVQYPNQGLQLLLKYTSAATLVKGINGFEAEFKKSETSFHLTKPSAHMSD | |
| (substituted) | AAEYFCAVSEDSNYQLIWGAGTKLIIKPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGFNL | |
| | LMTLRLWSS | |
| CDR1ß | GTSNPN | 2761 |
| CDR2ß | SVGIG | 2762 |
| CDR3ß | AYSPGLASDTQY | 2763 |
| Vß w/o signal peptide | QTIHQWPATLVQPVGSPLSLECTVEGTSNPNLYWYRQAAGRGLQLLFYSVGI | 2764 |
| (SignalP) | GQISSEVPQNLSASRPQDRQFILSSKKLLLSDSGFYLCAYSPGLASDTQYFG | |
| | PGTRLTVL | |
| Vß w/o signal peptide | SQTIHQWPATLVQPVGSPLSLECTVEGTSNPNLYWYRQAAGRGLQLLFYSVG | 2765 |
| (IMGT) | IGQISSEVPQNLSASRPQDRQFILSSKKLLLSDSGFYLCAYSPGLASDTQYF | |
| | GPGTRLTVL | |
| Vß | MXCSLLALLLGTFFGVRSQTIHQWPATLVQPVGSPLSLECTVEGTSNPNLYW | 2766 |
| | YRQAAGRGLQLLFYSVGIGQISSEVPQNLSASRPQDRQFILSSKKLLLSDSG | 2767 |
| | FYLCAYSPGLASDTQYFGPGTRLTVL | |
| | (X = any amino acid) | |
| ß chain w/WT signal | MLCSLLALLLGTFFGVRSQTIHQWPATLVQPVGSPLSLECTVEGTSNPNLYW | 2768 |
| peptide, Cß | YRQAAGRGLQLLFYSVGIGQISSEVPQNLSASRPQDRQFILSSKKLLLSDSG | |
| (substituted) | FYLCAYSPGLASDTQYFGPGTRLTVLEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| ß chain w/alternative | MACSLLALLLGTFFGVRSQTIHQWPATLVQPVGSPLSLECTVEGTSNPNLYW | 2769 |
| signal peptide, Cß | YRQAAGRGLQLLFYSVGIGQISSEVPQNLSASRPQDRQFILSSKKLLLSDSG | |
| (substituted) | FYLCAYSPGLASDTQYFGPGTRLTVLEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| ß chain w/alternative | MHCSLLALLLGTFFGVRSQTIHQWPATLVQPVGSPLSLECTVEGTSNPNLYW | 2770 |
| signal peptide, Cß | YRQAAGRGLQLLFYSVGIGQISSEVPQNLSASRPQDRQFILSSKKLLLSDSG | |
| (substituted) | FYLCAYSPGLASDTQYFGPGTRLTVLEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |

In some embodiments, TCR077 interacts with and/or is specific for the epidermal growth factor receptor (EGFR) tumor protein. In some embodiments, the peptide is from a neoantigen of EGFR. In some embodiments, the neoantigen has the amino acid changes E746-A750del relative to the wild type EGFR sequence. In some embodiments, TCR077 interacts with the neoantigen in the context of a heterodimer of HLA-DPA1*02:01 and HLA-DPB1*01:01, as described in International Publication No. WO 2019/213195, incorporated herein by reference in its entirety.

TABLE 6BZ
Amino acid sequences of TCR078.

| | | SEQ |
|---|---|---|
| Description | Sequence | ID NO: |
| CDR1α | TSINN | 1771 |
| CDR2α | IRSNERE | 1772 |
| CDR3α | ATDGETSGSRLT | 1773 |
| Vα without signal | QQGEEDPQALSIQEGFNATMNCSYKTSINNLQWYRQNSGRGLVHLILIRSNE | 1774 |
| peptide (SignalP) | REKHSGRLRVTLDTSKKSSSLLITASRAADTASYFCATDGETSGSRLTFGEG | |
| | TQLTVNP | |
| Vα without signal | SQQGEEDPQALSIQEGFNATMNCSYKTSINNLQWYRQNSGRGLVHLILIRSN | 1775 |
| peptide (IMGT) | EREKHSGRLRVTLDTSKKSSSLLITASRAADTASYFCATDGETSGSRLTFGE | |
| | GTQLTVNP | |
| Vα | MXTLLGVSLVILWLQLARVNSQQGEEDPQALSIQEGFNATMNCSYKTSINNL | 1776 |
| | QWYRQNSGRGLVHLILIRSNEREKHSGRLRVTLDTSKKSSSLLITASRAADT | 1777 |
| | ASYFCATDGETSGSRLTFGEGTQLTVNP | |
| | (X = any amino acid) | |
| α chain with WT signal | METLLGVSLVILWLQLARVNSQQGEEDPQALSIQEGFNATMNCSYKTSINNL | 1778 |
| peptide, Cα | QWYRQNSGRGLVHLILIRSNEREKHSGRLRVTLDTSKKSSSLLITASRAADT | |
| (substituted) | ASYFCATDGETSGSRLTFGEGTQLTVNPNIQNPEPAVYQLKDPRSQDSTLCL | |
| | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGFNL | |
| | LMTLRLWSS | |
| α chain with | MATLLGVSLVILWLQLARVNSQQGEEDPQALSIQEGFNATMNCSYKTSINNL | 1779 |
| alternative signal | QWYRQNSGRGLVHLILIRSNEREKHSGRLRVTLDTSKKSSSLLITASRAADT | |
| peptide, Cα | ASYFCATDGETSGSRLTFGEGTQLTVNPNIQNPEPAVYQLKDPRSQDSTLCL | |
| (substituted) | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGFNL | |
| | LMTLRLWSS | |
| α chain with | MHTLLGVSLVILWLQLARVNSQQGEEDPQALSIQEGFNATMNCSYKTSINNL | 1780 |
| alternative signal | QWYRQNSGRGLVHLILIRSNEREKHSGRLRVTLDTSKKSSSLLITASRAADT | |
| peptide, Cα | ASYFCATDGETSGSRLTFGEGTQLTVNPNIQNPEPAVYQLKDPRSQDSTLCL | |
| (substituted) | FTDFDSQINVPKTMESGTFITDKCVLDMKAMDSKSNGAIAWSNQTSFTCQDI | |
| | FKETNATYPSSDVPCDATLTEKSFETDMNLNFQNLLVIVLRILLLKVAGFNL | |
| | LMTLRLWSS | |
| CDR1ß | DFQATT | 2771 |
| CDR2ß | SNEGSKA | 2772 |
| CDR3ß | SASRGATGQPQH | 2773 |
| Vß without signal | AVVSQHPSWVICKSGTSVKIECRSLDFQATTMFWYRQFPKQSLMLMATSNEG | 2774 |
| peptide (SignalP) | SKATYEQGVEKDKFLINHASLTLSTLTVTSAHPEDSSFYICSASRGATGQPQ | |
| | HFGDGTRLSIL | |
| Vß without signal | GAVVSQHPSWVICKSGTSVKIECRSLDFQATTMFWYRQFPKQSLMLMATSNE | 2775 |
| peptide (IMGT) | GSKATYEQGVEKDKFLINHASLTLSTLTVTSAHPEDSSFYICSASRGATGQP | |
| | QHFGDGTRLSIL | |
| Vß | MXLLLLLLGPGSGLGAVVSQHPSWVICKSGTSVKIECRSLDFQATTMFWYRQ | 2776 |
| | FPKQSLMLMATSNEGSKATYEQGVEKDKFLINHASLTLSTLTVTSAHPEDSS | 2777 |
| | FYICSASRGATGQPQHFGDGTRLSIL | |
| | (X = any amino acid) | |
| ß chain with WT signal | MLLLLLLLGPGSGLGAVVSQHPSWVICKSGTSVKIECRSLDFQATTMFWYRQ | 2778 |
| peptide, Cß | FPKQSLMLMATSNEGSKATYEQGVEKDKFLINHASLTLSTLTVTSAHPEDSS | |
| (substituted) | FYICSASRGATGQPQHFGDGTRLSILEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| ß chain with alternative | MALLLLLLGPGSGLGAVVSQHPSWVICKSGTSVKIECRSLDFQATTMFWYRQ | 2779 |
| signal peptide, Cß | FPKQSLMLMATSNEGSKATYEQGVEKDKFLINHASLTLSTLTVTSAHPEDSS | |
| (substituted) | FYICSASRGATGQPQHFGDGTRLSILEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| ß chain with alternative | MHLLLLLLGPGSGLGAVVSQHPSWVICKSGTSVKIECRSLDFQATTMFWYRQ | 2789 |
| signal peptide, Cß | FPKQSLMLMATSNEGSKATYEQGVEKDKFLINHASLTLSTLTVTSAHPEDSS | |
| (substituted) | FYICSASRGATGQPQHFGDGTRLSILEDLRNVTPPKVSLFEPSKAEIANKQK | |
| | ATLVCLARGFFPDHVELSWWVNGKEVHSGVCTDPQAYKESNYSYCLSSRLRV | |
| | SATFWHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |

In some embodiments, TCR078 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR078 interacts with the neoantigen in the context of an HLA-DPA1*01:03 chain and an HLA-DPB1*03:01 chain, as described in International Publication No. WO 2021/173902, incorporated herein by reference in its entirety.

TABLE 6CA
Amino acid sequences of TCR079.

| | | SEQ |
|---|---|---|
| Description | Sequence | ID NO: |
| CDR1α | NSASDY | 1781 |
| CDR2α | IRSNMDK | 1782 |
| CDR3α | AERGRGGKLI | 1783 |
| Vα without signal | ESVGLHLPTLSVQEGDNSIINCAYSNSASDYFIWYKQESGKGPQFIIDIRSN | 1784 |
| peptide (SignalP) | MDKRQGQRVTVLLNKTVKHLSLQIAATQPGDSAVYFCAERGRGGKLIFGQGT | |
| | ELSVKP | |
| Vα without signal | GESVGLHLPTLSVQEGDNSIINCAYSNSASDYFIWYKQESGKGPQFIIDIRS | 1785 |
| peptide (IMGT) | NMDKRQGQRVTVLLNKTVKHLSLQIAATQPGDSAVYFCAERGRGGKLIFGQG | |
| | TELSVKP | |
| Vα | MXGIRALFMYLWLQLDWVSRGESVGLHLPTLSVQEGDNSIINCAYSNSASDY | 1786 |
| | FIWYKQESGKGPQFIIDIRSNMDKRQGQRVTVLLNKTVKHLSLQIAATQPGD | |
| | SAVYFCAERGRGGKLIFGQGTELSVKP | 1787 |
| | (X = any amino acid) | |
| α chain with WT signal | MAGIRALFMYLWLQLDWVSRGESVGLHLPTLSVQEGDNSIINCAYSNSASDY | 1788 |
| peptide, Cα | FIWYKQESGKGPQFIIDIRSNMDKRQGQRVTVLLNKTVKHLSLQIAATQPGD | |
| (substituted) | SAVYFCAERGRGGKLIFGQGTELSVKPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain with | MAGIRALFMYLWLQLDWVSRGESVGLHLPTLSVQEGDNSIINCAYSNSASDY | 1789 |
| alternative signal | FIWYKQESGKGPQFIIDIRSNMDKRQGQRVTVLLNKTVKHLSLQIAATQPGD | |
| peptide, Cα | SAVYFCAERGRGGKLIFGQGTELSVKPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain with | MHGIRALFMYLWLQLDWVSRGESVGLHLPTLSVQEGDNSIINCAYSNSASDY | 1790 |
| alternative signal | FIWYKQESGKGPQFIIDIRSNMDKRQGQRVTVLLNKTVKHLSLQIAATQPGD | |
| peptide, Cα | SAVYFCAERGRGGKLIFGQGTELSVKPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| CDR1ß | DFQATT | 2781 |
| CDR2ß | SNEGSKA | 2782 |
| CDR3B | SAGRASTDTQY | 2783 |
| Vß without signal | GSGLGAVVSQHPSWVICKSGTSVKIECRSLDFQATTMFWYRQFPKQSLMLMA | 2784 |
| peptide (SignalP) | TSNEGSKATYEQGVEKDKFLINHASLTLSTLTVTSAHPEDSSFYICSAGRAS | |
| | TDTQYFGPGTRLTVL | |
| Vß without signal | GAVVSQHPSWVICKSGTSVKIECRSLDFQATTMFWYRQFPKQSLMLMATSNE | 2785 |
| peptide (IMGT) | GSKATYEQGVEKDKFLINHASLTLSTLTVTSAHPEDSSFYICSAGRASTDTQ | |
| | YFGPGTRLTVL | |
| Vß | MXLLLLLLGPGISLLLPGSLAGSGLGAVVSQHPSWVICKSGTSVKIECRSLD | 2786 |
| | FQATTMFWYRQFPKQSLMLMATSNEGSKATYEQGVEKDKFLINHASLTLSTL | |
| | TVTSAHPEDSSFYICSAGRASTDTQYFGPGTRLTVL | 2787 |
| | (X = any amino acid) | |
| ß chain with WT signal | MLLLLLLLGPGISLLLPGSLAGSGLGAVVSQHPSWVICKSGTSVKIECRSLD | 2788 |
| peptide, Cß | FQATTMFWYRQFPKQSLMLMATSNEGSKATYEQGVEKDKFLINHASLTLSTL | |
| (substituted) | TVTSAHPEDSSFYICSAGRASTDTQYFGPGTRLTVLEDLRNVTPPKVSLFEP | |
| | SKAEIANKQKATLVCLARGFFPDHVELSWWVNGKEVHSGVCTDPQAYKESNY | |
| | SYCLSSRLRVSATFWHNPRNHFRCQVQFHGLSEEDKWPEGSPKPVTQNISAE | |
| | AWGRADCGITSASYQQGVLSATILYEILLGKATLYAVLVSTLVVMAMVKRKN | |
| | S | |
| ß chain with alternative | MALLLLLLGPGISLLLPGSLAGSGLGAVVSQHPSWVICKSGTSVKIECRSLD | 2789 |
| signal peptide, Cß | FQATTMFWYRQFPKQSLMLMATSNEGSKATYEQGVEKDKFLINHASLTLSTL | |
| (substituted) | TVTSAHPEDSSFYICSAGRASTDTQYFGPGTRLTVLEDLRNVTPPKVSLFEP | |
| | SKAEIANKQKATLVCLARGFFPDHVELSWWVNGKEVHSGVCTDPQAYKESNY | |
| | SYCLSSRLRVSATFWHNPRNHFRCQVQFHGLSEEDKWPEGSPKPVTQNISAE | |
| | AWGRADCGITSASYQQGVLSATILYEILLGKATLYAVLVSTLVVMAMVKRKN | |
| | S | |
| ß chain with alternative | MHLLLLLLGPGISLLLPGSLAGSGLGAVVSQHPSWVICKSGTSVKIECRSLD | 2790 |
| signal peptide, Cß | FQATTMFWYRQFPKQSLMLMATSNEGSKATYEQGVEKDKFLINHASLTLSTL | |
| (substituted) | TVTSAHPEDSSFYICSAGRASTDTQYFGPGTRLTVLEDLRNVTPPKVSLFEP | |
| | SKAEIANKQKATLVCLARGFFPDHVELSWWVNGKEVHSGVCTDPQAYKESNY | |
| | SYCLSSRLRVSATFWHNPRNHERCQVQFHGLSEEDKWPEGSPKPVTQNISAE | |
| | AWGRADCGITSASYQQGVLSATILYEILLGKATLYAVLVSTLVVMAMVKRKN | |
| | S | |

In some embodiments, TCR079 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR079 interacts with the neoantigen in the context of an HLA-DPA1*01:03 chain and an HLA-DPB1*03:01 chain, as described in International Publication No. WO 2021/173902, incorporated herein by reference in its entirety.

TABLE 6CB
Amino acid sequences of TCR080

| | | SEQ |
|---|---|---|
| Description | Sequence | ID NO: |
| CDR1α | NSASDY | 1791 |
| CDR2α | IRSNMDK | 1792 |
| CDR3α | AERGRGGKLI | 1793 |
| Vα without signal | ESVGLHLPTLSVQEGDNSIINCAYSNSASDYFIWYKQESGKGPQFIIDIRSN | 1794 |
| peptide (SignalP) | MDKRQGQRVTVLLNKTVKHLSLQIAATQPGDSAVYFCAERGRGGKLIFGQGT | |
| | ELSVKP | |
| Vα without signal | GESVGLHLPTLSVQEGDNSIINCAYSNSASDYFIWYKQESGKGPQFIIDIRS | 1795 |
| peptide (IMGT) | NMDKRQGQRVTVLLNKTVKHLSLQIAATQPGDSAVYFCAERGRGGKLIFGQG | |
| | TELSVKP | |
| Vα | MXGIRALFMYLWLQLDWVSRGESVGLHLPTLSVQEGDNSIINCAYSNSASDY | 1796 |
| | FIWYKQESGKGPQFIIDIRSNMDKRQGQRVTVLLNKTVKHLSLQIAATQPGD | 1797 |
| | SAVYFCAERGRGGKLIFGQGTELSVKP | |
| | (X = any amino acid) | |
| α chain with WT signal | MAGIRALFMYLWLQLDWVSRGESVGLHLPTLSVQEGDNSIINCAYSNSASDY | 1798 |
| peptide, Cα | FIWYKQESGKGPQFIIDIRSNMDKRQGQRVTVLLNKTVKHLSLQIAATQPGD | |
| (substituted) | SAVYFCAERGRGGKLIFGQGTELSVKPNIQNPEPAVYQLKDPRSQDSTLCLF | |
| | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain with | MAGIRALFMYLWLQLDWVSRGESVGLHLPTLSVQEGDNSIINCAYSNSASDY | 1799 |
| alternative signal | FIWYKQESGKGPQFIIDIRSNMDKRQGQRVTVLLNKTVKHLSLQIAATQPGD | |
| peptide, Cα | SAVYFCAERGRGGKLIFGQGTELSVKPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| α chain with | MHGIRALFMYLWLQLDWVSRGESVGLHLPTLSVQEGDNSIINCAYSNSASDY | 1800 |
| alternative signal | FIWYKQESGKGPQFIIDIRSNMDKRQGQRVTVLLNKTVKHLSLQIAATQPGD | |
| peptide, Cα | SAVYFCAERGRGGKLIFGQGTELSVKPNIQNPEPAVYQLKDPRSQDSTLCLF | |
| (substituted) | TDFDSQINVPKTMESGTFITDKCVLDMKAMDSKSNGAIAWSNQTSFTCQDIF | |
| | KETNATYPSSDVPCDATLTEKSFETDMNLNFQNLLVIVLRILLLKVAGFNLL | |
| | MTLRLWSS | |
| CDR1ß | DFQATT | 2791 |
| CDR2ß | SNEGSKA | 2792 |
| CDR3ß | SAGRASTDTQY | 2793 |
| VßB without signal | AVVSQHPSWVICKSGTSVKIECRSLDFQATTMFWYRQFPKQSLMLMATSNEG | 2794 |
| peptide (SignalP) | SKATYEQGVEKDKFLINHASLTLSTLTVTSAHPEDSSFYICSAGRASTDTQY | |
| | FGPGTRLTVL | |
| Vß without signal | GAVVSQHPSWVICKSGTSVKIECRSLDFQATTMFWYRQFPKQSLMLMATSNE | 2795 |
| peptide (IMGT) | GSKATYEQGVEKDKFLINHASLTLSTLTVTSAHPEDSSFYICSAGRASTDTQ | |
| | YFGPGTRLTVL | |
| Vß | MXLLLLLLGPGSGLGAVVSQHPSWVICKSGTSVKIECRSLDFQATTMFWYRQ | 2796 |
| | FPKQSLMLMATSNEGSKATYEQGVEKDKFLINHASLTLSTLTVTSAHPEDSS | 2797 |
| | FYICSAGRASTDTQYFGPGTRLTVL | |
| | (X = any amino acid) | |
| ß chain with WT signal | MLLLLLLLGPGSGLGAVVSQHPSWVICKSGTSVKIECRSLDFQATTMFWYRQ | 2798 |
| peptide, Cß | FPKQSLMLMATSNEGSKATYEQGVEKDKFLINHASLTLSTLTVTSAHPEDSS | |
| (substituted) | FYICSAGRASTDTQYFGPGTRLTVLEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| ß chain with alternative | MALLLLLLGPGSGLGAVVSQHPSWVICKSGTSVKIECRSLDFQATTMFWYRQ | 2799 |
| signal peptide, Cß | FPKQSLMLMATSNEGSKATYEQGVEKDKFLINHASLTLSTLTVTSAHPEDSS | |
| (substituted) | FYICSAGRASTDTQYFGPGTRLTVLEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |
| ß chain with alternative | MHLLLLLLGPGSGLGAVVSQHPSWVICKSGTSVKIECRSLDFQATTMFWYRQ | 2800 |
| signal peptide, Cß | FPKQSLMLMATSNEGSKATYEQGVEKDKFLINHASLTLSTLTVTSAHPEDSS | |
| (substituted) | FYICSAGRASTDTQYFGPGTRLTVLEDLRNVTPPKVSLFEPSKAEIANKQKA | |
| | TLVCLARGFFPDHVELSWWVNGKEVHSGVCTDPQAYKESNYSYCLSSRLRVS | |
| | ATFWHNPRNHFRCQVQFHGLSEEDKWPEGSPKPVTQNISAEAWGRADCGITS | |
| | ASYQQGVLSATILYEILLGKATLYAVLVSTLVVMAMVKRKNS | |

In some embodiments, TCR080 interacts with and/or is specific for KRAS. In some embodiments, the peptide is from a neoantigen of KRAS. In some embodiments, the neoantigen has the amino acid change G12V relative to the wild type KRAS sequence. In some embodiments, TCR080 interacts with the neoantigen in the context of an HLA-DPA1*01:03 chain and an HLA-DPB1*03:01 chain, as described in International Publication No. WO 2021/173902, incorporated herein by reference in its entirety.

The disclosure also provides for the use of other TCR Vα and Vβ sequences, as well as any other alpha or beta chains, in the polycistronic vectors, engineered cells or pharmaceutical compositions described herein. These TCR Vα and Vβ sequences and alpha or beta chains include those described in International Publication Nos. WO 2016/085904, WO 2017/048593, WO 2018/026691, WO 2019/060349, WO 2019/067243, WO 2019/070435, WO 2019/112941, WO 2019/213195, WO 2020/086827, WO 2020/154275, WO 2020/264269, WO 2021/163434, WO 2021/163477, and WO 2021/173902 incorporated by reference herein in their entireties.

The CDRs of a TCR disclosed herein can be defined using any art recognized numbering convention. Additionally or alternatively, the CDRs can be defined empirically, e.g., based upon structural analysis of the interaction of the TCR with a cognate antigen (e.g., a peptide or a peptide-MHC complex). In some embodiments, CDR3 of the TCR can further comprise an N-terminal cysteine and/or a C-terminal phenylalanine or tryptophan.

The TCRs disclosed herein can be used in any TCR structural format. For example, in certain embodiments, the TCR is a full-length TCR comprising a full-length α chain and a full-length β chain. The transmembrane regions (and optionally also the cytoplasmic regions) can be removed from a full-length TCR to produce a soluble TCR. Accordingly, in certain embodiments, the TCR is a soluble TCR lacking transmembrane and/or cytoplasmic region(s). The methods of producing soluble TCRs are well-known in the art. In some embodiments, the soluble TCR comprises an engineered disulfide bond that facilitates dimerization, see, e.g., U.S. Pat. No. 7,329,731, which is incorporated by reference herein in its entirety. In some embodiments, the soluble TCR is generated by fusing the extracellular domain of a TCR described herein to other protein domains, e.g., maltose binding protein, thioredoxin, human constant kappa domain, or leucine zippers, see, e.g., Løset et al., Front Oncol. 2014; 4: 378, which is incorporated by reference herein in its entirety. A single-chain TCR (scTCR) comprising Vα and Vβ linked by a peptide linker can also be generated. Such scTCRs can comprise Vα and Vβ, each linked to a TCR constant region. Alternatively, the scTCRs can comprise Vα and Vβ, where either the Vα, the Vβ, or both the Vα and Vβ are not linked to a TCR constant region. Exemplary scTCRs are described in PCT Publication Nos. WO 2003/020763, WO 2004/033685, and WO 2011/044186, each of which is incorporated by reference herein in its entirety. Furthermore, the TCRs disclosed herein can comprise two polypeptide chains (e.g., an a chain and a β chain) in which the chains have been engineered to each have a cysteine residue that can form an interchain disulfide bond. Accordingly, in certain embodiments, the TCRs disclosed herein comprise two polypeptide chains linked by an engineered disulfide bond. Exemplary TCRs having an engineered disulfide bond are described in U.S. Pat. Nos. 8,361,794 and 8,906,383, each of which is incorporated by reference herein in its entirety.

In certain embodiments, the TCRs disclosed herein comprise one or more chains (e.g., an α chain and/or a β chain) having a transmembrane region. In certain embodiments, the TCRs disclosed herein comprise two chains (e.g., an α chain and a β chain) having a transmembrane region. The transmembrane region can be the endogenous transmembrane region of that TCR chain, a variant of the endogenous transmembrane region, or a heterologous transmembrane region. In certain embodiments, the TCRs disclosed herein comprise an α chain and a β chain having endogenous transmembrane regions.

In certain embodiments, the TCRs disclosed herein comprise one or more chains (e.g., an α chain and/or a β chain) having a cytoplasmic region. In certain embodiments, the TCRs disclosed herein comprise two chains (e.g., an α chain and a β chain) each having a cytoplasmic region. The cytoplasmic region can be the endogenous cytoplasmic region of that TCR chain, variant of the endogenous cytoplasmic region, or a heterologous cytoplasmic region. In certain embodiments, the TCRs disclosed herein comprise two chains (e.g., an α chain and a β chain) where both chains have transmembrane regions, but one chain is lacking a cytoplasmic region. In certain embodiments, the TCRs disclosed herein comprise two chains (e.g., an α chain and a p chain) where both chains have endogenous transmembrane regions but lack an endogenous cytoplasmic region. In certain embodiments, the TCRs disclosed herein comprise an α chain and a β chain where both chains have endogenous transmembrane regions but lack an endogenous cytoplasmic region. In certain embodiments, the TCRs disclosed herein comprise a co-stimulatory signaling region from a co-stimulatory molecule; see, e.g., PCT Publication Nos.: WO 1996/018105, WO 1999/057268, and WO 2000/031239, and U.S. Pat. No. 7,052,906, all of which are incorporated herein by reference in their entireties.

In certain embodiments, the instant disclosure provides a polypeptide comprising an α chain variable region (Vα) and a β chain variable region (Vβ) of a TCR fused together. For example, such polypeptide may comprise, in order, the Vα and Vβ, or the Vβ and the Vα, optionally with a linker (e.g., a peptide linker) between the two regions. For example, a Furin and/or a 2A cleavage site (e.g., one of the sequences in Tables 2 or 3), or combinations thereof, may be used in the linker for the Vα/Vβ fusion polypeptide. In certain embodiments, the instant disclosure provides a polypeptide comprising an α chain and a β chain of a TCR fused together. For example, such polypeptide may comprise, in order, an α chain and a β chain, or a β chain and an a chain, optionally with a linker (e.g., a peptide linker) between the two chains. For example, a Furin and/or a 2A cleavage site (e.g., one of the sequences in Tables 2 or 3), or combinations thereof, may be used in the linker for the a/P fusion polypeptide. For example, a fusion polypeptide may comprise, from the N-terminus to the C-terminus: the a chain of a TCR, a furin cleavage site, a 2A cleavage site, and the β chain of the TCR. In certain embodiments, the polypeptide comprises, from the N-terminus to the C-terminus: the β chain of a TCR, a furin cleavage site, a 2A element, and the a chain of the TCR.

### 1.3 IL-15/IL-15Rα Fusion Proteins

The disclosure also provides recombinant vectors that include cytokines. In some embodiments, the cytokine is an interleukin. In some embodiments, the cytokine is membrane bound. In some embodiments, the cytokine is a fusion protein comprising a soluble cytokine, or a functional fragment or functional variant thereof, operably linked to a cognate receptor of the cytokine, or a functional fragment or functional variant thereof, optionally a membrane-bound form thereof. In some embodiments, the fusion protein comprises human IL-15 (hIL-15) operably linked to human IL-15Rα (hIL-15Rα). In membrane-bound form, this fusion protein is referred to herein as membrane bound IL-15 (mbIL15). In some embodiments, hIL-15 is directly operably linked to hIL-15Rα. In some embodiments, hIL-15 is indirectly operably linked to hIL-15Rα. In some embodiments, hIL-15 is indirectly operably linked to hIL-15Rα via a peptide linker.

In some embodiments, the peptide linker comprises the amino acid sequence of SEQ ID NO: 81, or an amino acid sequence comprising 1, 2, 3, 4 or 5 amino acid modifications to the amino acid sequence of SEQ ID NO: 81. In some embodiments, the linker comprises the amino acid sequence of SEQ ID NO: 81. In some embodiments, the amino acid of the linker consists of the amino acid sequence of SEQ ID NO: 81, or an amino acid sequence comprising 1, 2, 3, 4 or 5 amino acid modifications to the amino acid sequence of SEQ ID NO: 81. In some embodiments, the amino acid of the linker consists of the amino acid sequence of SEQ ID NO: 81.

In some embodiments, the linker is encoded by a polynucleotide sequence at least 95%, 96%, 97%, 98%, 99%, or 100% identical to the polynucleotide sequence of SEQ ID NO: 82. In some embodiments, the linker is encoded by the polynucleotide sequence of SEQ ID NO: 82. In some embodiments, hIL-15 comprises an amino acid sequence at least 95%, 96%, 97%, 98%, 99% or 100% identical to the amino acid sequence of SEQ ID NO: 76. In some embodiments, hIL-15 comprises the amino acid sequence of SEQ ID NO: 76. In some embodiments, the amino acid sequence of hIL-15 consists of a sequence at least 95%, 96%, 97%, 98%, 99% or 100% identical to the amino acid sequence of SEQ ID NO: 76. In some embodiments, the amino acid sequence of hIL-15 consists of the amino acid sequence of SEQ ID NO: 76.

In some embodiments, hIL-15 is encoded by a polynucleotide sequence at least 75%, 80%, 85%, 90%, 95%, 96%, 97%, 98%, 99%, or 100% identical to the polynucleotide sequence of SEQ ID NO: 77. In some embodiments, hIL-15 is encoded by the polynucleotide sequence of SEQ ID NO: 77.

In some embodiments, hIL-15Rα comprises an amino acid sequence at least 95%, 96%, 97%, 98%, 99% or 100% identical to the amino acid sequence of SEQ ID NO: 78. In some embodiments, hIL-15Rα comprises the amino acid sequence of SEQ ID NO: 78. In some embodiments, the amino acid sequence of hIL-15Rα consists of a sequence at least 95%, 96%, 97%, 98%, 99% or 100% identical to the amino acid sequence of SEQ ID NO: 78. In some embodiments, the amino acid sequence of hIL-15Rα consists of the amino acid sequence of SEQ ID NO: 78.

In some embodiments, hIL-15Rα is encoded by a polynucleotide sequence at least 75%, 80%, 85%, 90%, 95%, 96%, 97%, 98%, 99%, or 100% identical to the polynucleotide sequence of SEQ ID NO: 79. In some embodiments, hIL-15Rα is encoded by the polynucleotide sequence of SEQ ID NO: 79

In some embodiments, the fusion protein comprises an amino acid sequence at least 95%, 96%, 97%, 98%, 99% or 100% identical to SEQ ID NO: 70 or 73. In some embodiments, the fusion protein comprises an amino acid sequence at least 95%, 96%, 97%, 98%, 99% or 100% identical to SEQ ID NO: 70. In some embodiments, the fusion protein comprises an amino acid sequence at least 95%, 96%, 97%, 98%, 99% or 100% identical to SEQ ID NO: 73. In some embodiments, the fusion protein comprises the amino acid sequence of SEQ ID NO: 70 or 73. In some embodiments, the fusion protein comprises the amino acid sequence of SEQ ID NO: 70. In some embodiments, the fusion protein comprises the amino acid sequence of SEQ ID NO: 73.

In some embodiments, the amino acid sequence of the fusion protein consists of a sequence at least 95%, 96%, 97%, 98%, 99% or 100% identical to the amino acid sequence of SEQ ID NO: 70 or 73. In some embodiments, the amino acid sequence of the fusion protein consists of a sequence at least 95%, 96%, 97%, 98%, 99% or 100% identical to the amino acid sequence of SEQ ID NO: 70. In some embodiments, the amino acid sequence of the fusion protein consists of a sequence at least 95%, 96%, 97%, 98%, 99% or 100% identical to the amino acid sequence of SEQ ID NO: 73. In some embodiments, the amino acid sequence of the fusion protein consists of the amino acid sequence of SEQ ID NO: 70 or 73. In some embodiments, the amino acid sequence of the fusion protein consists of the amino acid sequence of SEQ ID NO: 70. In some embodiments, the amino acid sequence of the fusion protein consists of the amino acid sequence of SEQ ID NO: 73.

In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 71 or 74. In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 71. In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 74.

In some embodiments, the fusion protein is encoded by the polynucleotide sequence of SEQ ID NO: 71 or 74. In some embodiments, the fusion protein is encoded by the polynucleotide sequence of SEQ ID NO: 71. In some embodiments, the fusion protein is encoded by the polynucleotide sequence of SEQ ID NO: 74.

Exemplary cytokine fusion proteins and components thereof are disclosed in Table 7. Additional exemplary mbIL15 fusions are disclosed in Hurton et al., “Tethered IL-15 augments antitumor activity and promotes a stem-cell memory subset in tumor-specific T cells,” PNAS, 113(48) E7788-E7797 (2016), the entire contents of which are incorporated by reference herein.

The amino acid sequence and polynucleotide sequence of exemplary cytokine fusion proteins and component polypeptides are provided in Table 7, herein.

TABLE 7
Amino acid and polynucleotide sequences of exemplary IL-15/IL-15RRα fusion
proteins and components thereof.

| Description | Sequence | SEQ ID NO |
|---|---|---|
| mbIL15 (with N- | MDWTWILFLVAAATRVHSNWVNVISDLKKIEDLIQSMHIDATLYTESDVHPSCK | 70 |
| terminal signal | VTAMKCFLLELQVISLESGDASIHDTVENLIILANNSLSSNGNVTESGCKECEE | |
| sequence) | LEEKNIKEFLQSFVHIVQMFINTSSGGGSGGGGSGGGGSGGGGSGGGSLQITCP | |
| (exemplary amino | PPMSVEHADIWVKSYSLYSRERYICNSGFKRKAGTSSLTECVLNKATNVAHWTT | |
| acid sequence) | PSLKCIRDPALVHQRPAPPSTVTTAGVTPQPESLSPSGKEPAASSPSSNNTAAT | |
| | TAAIVPGSQLMPSKSPSTGTTEISSHESSHGTPSQTTAKNWELTASASHQPPGV | |
| | YPQGHSDTTVAISTSTVLLCGLSAVSLLACYLKSRQTPPLASVEMEAMEALPVT | |
| | WGTSSRDEDLENCSHHL | |
| mbIL15 (with N- | ATGGATTGGACCTGGATTCTGTTTCTGGTGGCCGCTGCCACAAGAGTGCACAGC | 71 |
| terminal signal | AACTGGGTGAATGTGATCAGCGACCTGAAGAAGATCGAGGATCTGATCCAGAGC | |
| sequence) | ATGCACATTGATGCCACCCTGTACACAGAATCTGATGTGCACCCTAGCTGTAAA | |
| (exemplary | GTGACCGCCATGAAGTGTTTTCTGCTGGAGCTGCAGGTGATTTCTCTGGAAAGC | |
| nucleotide | GGAGATGCCTCTATCCACGACACAGTGGAGAATCTGATCATCCTGGCCAACAAT | |
| sequence) | AGCCTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGAGGA | |
| | GGAGGCAGTGGAGGCGGAGGATCTGGCGGAGGATCTCTGCAGATTACATGCCCT | |
| | CCTCCAATGTCTGTGGAGCACGCCGATATTTGGGTGAAGTCCTACAGCCTGTAC | |
| | AGCAGAGAGAGATACATCTGCAACAGCGGCTTTAAGAGAAAGGCCGGCACCTCT | |
| | TCTCTGACAGAGTGCGTGCTGAATAAGGCCACAAATGTGGCCCACTGGACAACA | |
| | CCTAGCCTGAAGTGCATTAGAGATCCTGCCCTGGTCCACCAGAGGCCTGCCCCT | |
| | CCATCTACAGTGACAACAGCCGGAGTGACACCTCAGCCTGAATCTCTGAGCCCT | |
| | TCTGGAAAAGAACCTGCCGCCAGCTCTCCTAGCTCTAATAATACCGCCGCCACA | |
| | ACAGCCGCCATTGTGCCTGGATCTCAGCTGATGCCTAGCAAGTCTCCTAGCACA | |
| | GGCACAACAGAGATCAGCAGCCACGAATCTTCTCACGGAACACCTTCTCAGACC | |
| | ACCGCCAAGAATTGGGAGCTGACAGCCTCTGCCTCTCACCAGCCTCCAGGAGTG | |
| | TATCCTCAGGGCCACTCTGATACAACAGTGGCCATCAGCACATCTACAGTGCTG | |
| | CTGTGTGGACTGTCTGCCGTGTCTCTGCTGGCCTGTTACCTGAAGTCTAGACAG | |
| | ACACCTCCTCTGGCCTCTGTGGAGATGGAGGCCATGGAAGCCCTGCCTGTGACA | |
| | TGGGGAACAAGCAGCAGAGATGAAGACCTGGAGAATTGTTCTCACCACCTG | |
| mbIL15 (without | NWVNVISDLKKIEDLIQSMHIDATLYTESDVHPSCKVTAMKCFLLELQVISLES | 73 |
| N-terminal signal | GDASIHDTVENLIILANNSLSSNGNVTESGCKECEELEEKNIKEFLQSFVHIVQ | |
| sequence) | MFINTSSGGGSGGGGSGGGGSGGGGSGGGSLQITCPPPMSVEHADIWVKSYSLY | |
| (exemplary amino | SRERYICNSGFKRKAGTSSLTECVLNKATNVAHWTTPSLKCIRDPALVHQRPAP | |
| acid sequence) | PSTVTTAGVTPQPESLSPSGKEPAASSPSSNNTAATTAAIVPGSQLMPSKSPST | |
| | GTTEISSHESSHGTPSQTTAKNWELTASASHQPPGVYPQGHSDTTVAISTSTVL | |
| | LCGLSAVSLLACYLKSRQTPPLASVEMEAMEALPVTWGTSSRDEDLENCSHHL | |
| mbIL15 (without | AACTGGGTGAATGTGATCAGCGACCTGAAGAAGATCGAGGATCTGATCCAGAGC | 74 |
| N-terminal signal | ATGCACATTGATGCCACCCTGTACACAGAATCTGATGTGCACCCTAGCTGTAAA | |
| sequence) | GTGACCGCCATGAAGTGTTTTCTGCTGGAGCTGCAGGTGATTTCTCTGGAAAGC | |
| (exemplary | GGAGATGCCTCTATCCACGACACAGTGGAGAATCTGATCATCCTGGCCAACAAT | |
| nucleotide | AGCCTGAGCAGCAATGGCAATGTGACAGAGTCTGGCTGTAAGGAGTGTGAGGAG | |
| sequence) | CTGGAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGAGGA | |
| | GGAGGCAGTGGAGGCGGAGGATCTGGCGGAGGATCTCTGCAGATTACATGCCCT | |
| | CCTCCAATGTCTGTGGAGCACGCCGATATTTGGGTGAAGTCCTACAGCCTGTAC | |
| | AGCAGAGAGAGATACATCTGCAACAGCGGCTTTAAGAGAAAGGCCGGCACCTCT | |
| | TCTCTGACAGAGTGCGTGCTGAATAAGGCCACAAATGTGGCCCACTGGACAACA | |
| | CCTAGCCTGAAGTGCATTAGAGATCCTGCCCTGGTCCACCAGAGGCCTGCCCCT | |
| | CCATCTACAGTGACAACAGCCGGAGTGACACCTCAGCCTGAATCTCTGAGCCCT | |
| | TCTGGAAAAGAACCTGCCGCCAGCTCTCCTAGCTCTAATAATACCGCCGCCACA | |
| | ACAGCCGCCATTGTGCCTGGATCTCAGCTGATGCCTAGCAAGTCTCCTAGCACA | |
| | GGCACAACAGAGATCAGCAGCCACGAATCTTCTCACGGAACACCTTCTCAGACC | |
| | ACCGCCAAGAATTGGGAGCTGACAGCCTCTGCCTCTCACCAGCCTCCAGGAGTG | |
| | TATCCTCAGGGCCACTCTGATACAACAGTGGCCATCAGCACATCTACAGTGCTG | |
| | CTGTGTGGACTGTCTGCCGTGTCTCTGCTGGCCTGTTACCTGAAGTCTAGACAG | |
| | ACACCTCCTCTGGCCTCTGTGGAGATGGAGGCCATGGAAGCCCTGCCTGTGACA | |
| | TGGGGAACAAGCAGCAGAGATGAAGACCTGGAGAATTGTTCTCACCACCTG | |
| Soluble hIL-15 | NWVNVISDLKKIEDLIQSMHIDATLYTESDVHPSCKVTAMKCFLLELQVISLES | 76 |
| (exemplary amino | GDASIHDTVENLIILANNSLSSNGNVTESGCKECEELEEKNIKEFLQSFVHIVQ | |
| acid sequence) | MFINTS | |
| Soluble hIL-15 | AACTGGGTGAATGTGATCAGCGACCTGAAGAAGATCGAGGATCTGATCCAGAGC | 77 |
| (exemplary | ATGCACATTGATGCCACCCTGTACACAGAATCTGATGTGCACCCTAGCTGTAAA | |
| nucleotide | GTGACCGCCATGAAGTGTTTTCTGCTGGAGCTGCAGGTGATTTCTCTGGAAAGC | |
| sequence) | GGAGATGCCTCTATCCACGACACAGTGGAGAATCTGATCATCCTGGCCAACAAT | |
| | AGCCTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTGCAG | |
| | ATGTTCATCAATACAAGC | |
| hIL-15Rα | ITCPPPMSVEHADIWVKSYSLYSRERYICNSGFKRKAGTSSLTECVLNKATNVA | 78 |
| (exemplary amino | HWTTPSLKCIRDPALVHQRPAPPSTVTTAGVTPQPESLSPSGKEPAASSPSSNN | |
| acid sequence) | TAATTAAIVPGSQLMPSKSPSTGTTEISSHESSHGTPSQTTAKNWELTASASHQ | |
| | PPGVYPQGHSDTTVAISTSTVLLCGLSAVSLLACYLKSRQTPPLASVEMEAMEA | |
| | LPVTWGTSSRDEDLENCSHHL | |
| hIL-15Rα | ATTACATGCCCTCCTCCAATGTCTGTGGAGCACGCCGATATTTGGGTGAAGTCC | 79 |
| (exemplary | TACAGCCTGTACAGCAGAGAGAGATACATCTGCAACAGCGGCTTTAAGAGAAAG | |
| nucleotide | GCCGGCACCTCTTCTCTGACAGAGTGCGTGCTGAATAAGGCCACAAATGTGGCC | |
| sequence) | CACTGGACAACACCTAGCCTGAAGTGCATTAGAGATCCTGCCCTGGTCCACCAG | |
| | AGGCCTGCCCCTCCATCTACAGTGACAACAGCCGGAGTGACACCTCAGCCTGAA | |
| | TCTCTGAGCCCTTCTGGAAAAGAACCTGCCGCCAGCTCTCCTAGCTCTAATAAT | |
| | ACCGCCGCCACAACAGCCGCCATTGTGCCTGGATCTCAGCTGATGCCTAGCAAG | |
| | TCTCCTAGCACAGGCACAACAGAGATCAGCAGCCACGAATCTTCTCACGGAACA | |
| | CCTTCTCAGACCACCGCCAAGAATTGGGAGCTGACAGCCTCTGCCTCTCACCAG | |
| | CCTCCAGGAGTGTATCCTCAGGGCCACTCTGATACAACAGTGGCCATCAGCACA | |
| | TCTACAGTGCTGCTGTGTGGACTGTCTGCCGTGTCTCTGCTGGCCTGTTACCTG | |
| | AAGTCTAGACAGACACCTCCTCTGGCCTCTGTGGAGATGGAGGCCATGGAAGCC | |
| | CTGCCTGTGACATGGGGAACAAGCAGCAGAGATGAAGACCTGGAGAATTGTTCT | |
| | CACCACCTG | |
| Linker | SGGGSGGGGSGGGGSGGGGSGGGSLQ | 81 |
| (exemplary amino | | |
| acid sequence) | | |
| Linker | TCTGGCGGAGGATCTGGAGGAGGCGGATCTGGAGGAGGAGGCAGTGGAGGCGGA | 82 |
| (exemplary | GGATCTGGCGGAGGATCTCTGCAG | |
| nucleotide | | |
| sequence) | | |
| IgE N-terminal | MDWTWILFLVAAATRVHS | 83 |
| signal sequence | | |
| (exemplary amino | | |
| acid sequence) | | |
| IgE N-terminal | ATGGATTGGACCTGGATTCTGTTTCTGGTGGCCGCTGCCACAAGAGTGCACAGC | 84 |
| signal sequence | | |
| (exemplary | | |
| nucleotide | | |
| sequence) | | |

### 1.4 Marker Proteins

The marker proteins described herein function to allow for the selective depletion of cells contacted with the recombinant vector disclosed herein (e.g., “recombinant cells”) in vivo, through the administration of an agent, e.g., an antibody, that specifically binds to the marker protein and may mediate or catalyze killing of a recombinant cell. In some embodiments, marker proteins are expressed on the surface of the recombinant cell.

In some embodiments, the marker protein comprises the extracellular domain of a cell surface protein, or a functional fragment or functional variant thereof. In some embodiments, the cell surface protein is human epidermal growth factor receptor 1 (hHER1). In some embodiments, the marker protein comprises a truncated HER1 protein that is able to be bound by an anti-hHER1 antibody. In some embodiments, the marker protein comprises a variant of a truncated hHER1 protein that is able to be bound by an anti-hHER1 antibody. In some embodiments, the hHER1 marker protein provides a safety mechanism by allowing for depletion of infused recombinant cells through administering an antibody that recognizes the hHER1 marker protein expressed on the surface of recombinant cells. An exemplary antibody that binds the hHER1 marker protein is cetuximab.

In some embodiments, the hHER1 marker protein comprises from N terminus to C terminus: domain III of hHER1, or a functional fragment or functional variant thereof; an N-terminal portion of domain IV of hHER1; and the transmembrane region of human CD28.

In some embodiments, domain III of hHER1 comprises the amino acid sequence of SEQ ID NO: 104; or the amino acid sequence of SEQ ID NO: 104, comprising 1, 2, or 3 amino acid modifications. In some embodiments, the amino acid sequence of domain III of hHER1 consists of the amino acid sequence of SEQ ID NO: 104; or the amino acid sequence of SEQ ID NO: 10, comprising 1, 2, or 3 amino acid modifications.

In some embodiments, the N-terminal portion of domain IV of hHER1 comprises amino acids 1-40, 1-39, 1-38, 1-37, 1-36, 1-35, 1-34, 1-33, 1-32, 1-31, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, or 1-10 of SEQ ID NO: 105. In some embodiments, the C terminus of domain III of hHER1 is directly fused to the N terminus of the N-terminal portion of domain IV of hHER1.

In some embodiments, the C terminus of the N-terminal portion of domain IV of hHER1 is indirectly fused to the N terminus of the CD28 transmembrane domain via a peptide linker. In some embodiments, the peptide linker comprises glycine and serine amino acid residues. In some embodiments, the peptide linker is from about 5-25, 5-20, 5-15, 5-10, 10-20, or 10-15 amino acids in length.

In some embodiments, the peptide linker comprises the amino acid sequence of SEQ ID NO: 108, or an amino acid sequence comprising 1, 2, 3, 4 or 5 amino acid modifications to the amino acid sequence of SEQ ID NO: 108. In some embodiments, the peptide linker comprises the amino acid sequence of SEQ ID NO: 108. In some embodiments, the amino acid sequence of the peptide linker consists of the amino acid sequence of SEQ ID NO: 108, or an amino acid sequence comprising 1, 2, 3, 4 or 5 amino acid modifications to the amino acid sequence of SEQ ID NO: 108. In some embodiments, the amino acid sequence of the peptide linker consists of the amino acid sequence of SEQ ID NO: 108.

In some embodiments, the marker protein comprises an amino acid sequence at least 95%, 96%, 97%, 98%, 99% or 100% identical to the amino acid sequence of SEQ ID NO: 100, 103, 112, or 113. In some embodiments, the marker protein comprises an amino acid sequence at least 95%, 96%, 97%, 98%, 99% or 100% identical to the amino acid sequence of SEQ ID NO: 100. In some embodiments, the marker protein comprises an amino acid sequence at least 95%, 96%, 97%, 98%, 99% or 100% identical to the amino acid sequence of SEQ ID NO: 103. In some embodiments, the marker protein comprises an amino acid sequence at least 95%, 96%, 97%, 98%, 99% or 100% identical to the amino acid sequence of SEQ ID NO: 112. In some embodiments, the marker protein comprises an amino acid sequence at least 95%, 96%, 97%, 98%, 99% or 100% identical to the amino acid sequence of SEQ ID NO: 113.

In some embodiments, the marker protein comprises the amino acid sequence of SEQ ID NO: 100 or 103. In some embodiments, the marker protein comprises the amino acid sequence of SEQ ID NO: 100. In some embodiments, the marker protein comprises the amino acid sequence of SEQ ID NO: 103.

In some embodiments, the marker protein consists of an amino acid sequence at least 95%, 96%, 97%, 98%, 99% or 100% identical to the amino acid sequence of SEQ ID NO: 100, 103, 112, or 113. In some embodiments, the marker protein consists of an amino acid sequence at least 95%, 96%, 97%, 98%, 99% or 100% identical to the amino acid sequence of SEQ ID NO: 100. In some embodiments, the marker protein consists of an amino acid sequence at least 95%, 96%, 97%, 98%, 99% or 100% identical to the amino acid sequence of SEQ ID NO: 103. In some embodiments, the marker protein consists of an amino acid sequence at least 95%, 96%, 97%, 98%, 99% or 100% identical to the amino acid sequence of SEQ ID NO: 112. In some embodiments, the marker protein consists of an amino acid sequence at least 95%, 96%, 97%, 98%, 99% or 100% identical to the amino acid sequence of SEQ ID NO: 113.

In some embodiments, the marker protein consists of the amino acid sequence of SEQ ID NO: 100, 103, 112, or 113. In some embodiments, the marker protein consists of the amino acid sequence of SEQ ID NO: 100. In some embodiments, the marker protein consists of the amino acid sequence of SEQ ID NO: 103. In some embodiments, the marker protein consists of the amino acid sequence of SEQ ID NO: 112. In some embodiments, the marker protein consists of the amino acid sequence of SEQ ID NO: 113.

In some embodiments, the marker protein is derived from human CD20 (hCD20). In some embodiments, the marker protein comprises a truncated hCD20 protein that comprises the extracellular region (hCD20t), or a functional fragment or functional variant thereof. In some embodiments, the hCD20 marker protein provides a safety mechanism by allowing for depletion of infused recombinant cells through administering an antibody that recognizes the hCD20 marker protein expressed on the surface of recombinant cells. An exemplary antibody that binds the hCD20 marker protein is rituximab.

The amino acid sequences of exemplary marker proteins are provided in Table 8, herein.

TABLE 8
Amino acid sequences of exemplary marker proteins.

| Description | Amino Acid Sequence | SEQ ID NO |
|---|---|---|
| HER1t (with N-terminal | MRLPAQLLGLLMLWVPGSSGRKVCNGIGIGEFKDSLSINATNI | 100 |
| signal sequence) (exemplary | KHFKNCTSISGDLHILPVAFRGDSFTHTPPLDPQELDILKTVK | |
| amino acid sequence) | EITGFLLIQAWPENRTDLHAFENLEIIRGRTKQHGQFSLAVVS | |
| | LNITSLGLRSLKEISDGDVIISGNKNLCYANTINWKKLFGTSG | |
| | QKTKIISNRGFNSCKATGQVCHALCSPEGCWGPEPRDCVSGGG | |
| | GSGGGGSGGGGSGGGGSFWVLVVVGGVLACYSLLVTVAFIIFW | |
| | VRSKRS | |
| HER1t (without N-terminal | RKVCNGIGIGEFKDSLSINATNIKHFKNCTSISGDLHILPVAF | 103 |
| signal sequence) (exemplary | RGDSFTHTPPLDPQELDILKTVKEITGFLLIQAWPENRTDLHA | |
| amino acid sequence) | FENLEIIRGRTKQHGQFSLAVVSLNITSLGLRSLKEISDGDVI | |
| | ISGNKNLCYANTINWKKLFGTSGQKTKIISNRGFNSCKATGQV | |
| | CHALCSPEGCWGPEPRDCVSGGGGSGGGGSGGGGSGGGGSFWV | |
| | LVVVGGVLACYSLLVTVAFIIFWVRSKRS | |
| Domain III of hHER1 | RKVCNGIGIGEFKDSLSINATNIKHFKNCTSISGDLHILPVAF | 104 |
| (exemplary amino acid | RGDSFTHTPPLDPQELDILKTVKEITGFLLIQAWPENRTDLHA | |
| sequence) | FENLEIIRGRTKQHGQFSLAVVSLNITSLGLRSLKEISDGDVI | |
| | ISGNKNLCYANTINWKKLFGTSGQKTKIISNRGFNSCKATGQ | |
| Domain IV of hHER1 | VCHALCSPEGCWGPEPRDCVSCRNVSRGRECVDKCNLLEGEPR | 105 |
| (exemplary amino acid | EFVENSECIQCHPECLPQAMNITCTGRGPDNCIQCAHYIDGPH | |
| sequence) | CVKTCPAGVMGFNNTLVWKYADAGHVCHLCHPNCTYGCTGPGL | |
| | EGCPTNGPKIPS | |
| Truncated domain IV of | VCHALCSPEGCWGPEPRDCVS | 106 |
| hHER1 (exemplary amino | | |
| sequence) | | |
| CD28 transmembrane domain | FWVLVVVGGVLACYSLLVTVAFIIFWVRSKRS | 107 |
| (exemplary amino acid | | |
| sequence) | | |
| Linker (exemplary amino acid | GGGGSGGGGSGGGGSGGGGS | 108 |
| sequence) | | |
| Igκ N-terminal signal | MRLPAQLLGLLMLWVPGSSG | 109 |
| sequence (exemplary amino | | |
| acid sequence) | | |
| Igκ Variant 1 N-terminal | MRMRLPAQLLGLLMLWVPGSSG | 110 |
| signal sequence (exemplary | | |
| amino acid sequence) | | |
| Igκ Variant 2 N-terminal | PRMRLPAQLLGLLMLWVPGSSG | 111 |
| signal sequence (exemplary | | |
| amino acid sequence) | | |
| HER1t-2 (with N-terminal | MRLPAQLLGLLMLWVPGSSGRKVCNGIGIGEFKDSLSINATNI | 112 |
| signal sequence) (exemplary | KHFKNCTSISGDLHILPVAFRGDSFTHTPPLDPQELDILKTVK | |
| amino acid sequence) | EITGFLLIQAWPENRTDLHAFENLEIIRGRTKQHGQFSLAVVS | |
| | LNITSLGLRSLKEISDGDVIISGNKNLCYANTINWKKLFGTSG | |
| | QKTKIISNRGFNSCKATGQVCHALCSPEGCWGPEPRDCVSCRN | |
| | VSRGRECVDKCNLLEGEPREFVENSECIQCHPECLPQAMNITC | |
| | TGRGPDNCIQCAHYIDGPHCVKTCPAGVMGFNNTLVWKYADAG | |
| | HVCHLCHPNCTYGCTGPGLEGCPTNGPKIPSIATGMVGALLLL | |
| | LVVALGIGLFM | |
| HER1t-2 (without N-terminal | RKVCNGIGIGEFKDSLSINATNIKHFKNCTSISGDLHILPVAF | 113 |
| signal sequence) (exemplary | RGDSFTHTPPLDPQELDILKTVKEITGFLLIQAWPENRTDLHA | |
| amino acid sequence) | FENLEIIRGRTKQHGQFSLAVVSLNITSLGLRSLKEISDGDVI | |
| | ISGNKNLCYANTINWKKLFGTSGQKTKIISNRGFNSCKATGQV | |
| | CHALCSPEGCWGPEPRDCVSCRNVSRGRECVDKCNLLEGEPRE | |
| | FVENSECIQCHPECLPQAMNITCTGRGPDNCIQCAHYIDGPHC | |
| | VKTCPAGVMGFNNTLVWKYADAGHVCHLCHPNCTYGCTGPGLE | |
| | GCPTNGPKIPSIATGMVGALLLLLVVALGIGLFM | |
| hCD20 (full length) | MTTPRNSVNGTFPAEPMKGPIAMQSGPKPLFRRMSSLVGPTQS | 114 |
| (exemplary amino acid | FFMRESKTLGAVQIMNGLFHIALGGLLMIPAGIYAPICVTVWY | |
| sequence) | PLWGGIMYIISGSLLAATEKNSRKCLVKGKMIMNSLSLFAAIS | |
| | GMILSIMDILNIKISHFLKMESLNFIRAHTPYINIYNCEPANP | |
| | SEKNSPSTQYCYSIQSLFLGILSVMLIFAFFQELVIAGIVENE | |
| | WKRTCSRPKSNIVLLSAEEKKEQTIEIKEEVVGLTETSSQPKN | |
| | EEDIEIIPIQEEEEEETETNFPEPPQDQESSPIENDSSP | |
| hCD20t-1 (exemplary amino | MTTPRNSVNGTFPAEPMKGPIAMQSGPKPLFRRMSSLVGPTQS | 115 |
| acid sequence) | FFMRESKTLGAVQIMNGLFHIALGGLLMIPAGIYAPICVTVWY | |
| | PLWGGIMYIISGSLLAATEKNSRKCLVKGKMIMNSLSLFAAIS | |
| | GMILSIMDILNIKISHFLKMESLNFIRAHTPYINIYNCEPANP | |
| | SEKNSPSTQYCYSIQSLFLGILSVMLIFAFFQELVIAGIVENE | |
| | WKRTCSRPKSNIVLLSAEEKKEQTIEIKEEVVGLTETSSQPKN | |
| | EEDIE | |

### 1.5 Vectors

In one aspect, provided herein are T cells transduced by electroporation with recombinant vectors comprising a polycistronic expression cassette that comprises at least three cistrons. In some embodiments, the polycistronic expression cassette comprises at least 4, 5, or 6 cistrons. In some embodiments, the polycistronic expression cassette comprises 3 cistrons. In some embodiments, the polycistronic expression cassette comprises 4 cistrons. In some embodiments, the polycistronic expression cassette comprises 5 cistrons. In some embodiments, the polycistronic expression cassette comprises 6 cistrons.

In some embodiments, the vector is a non-viral vector. Exemplary non-viral vectors include, but are not limited to, plasmid DNA, transposons, episomal plasmids, minicircles, ministrings, and oligonucleotides (e.g., mRNA, naked DNA). In some embodiments, the polycistronic vector is a DNA plasmid vector.

In some embodiments, the vector is a viral vector. Viral vectors can be replication competent or replication incompetent. Viral vectors can be integrating or non-integrating. A number of viral based systems have been developed for gene transfer into mammalian cells, and a suitable viral vector can be selected by a person of ordinary skill in the art. Exemplary viral vectors include, but are not limited to, adenovirus vectors (e.g., adenovirus 5), adeno-associated virus (AAV) vectors (e.g., AAV2, 3, 5, 6, 8, 9), retrovirus vectors (MMSV, MSCV), lentivirus vectors (e.g., HIV-1, HIV-2), gammaretrovirus vectors, herpes virus vectors (e.g., HSV1, HSV2), alphavirus vectors (e.g., SFV, SIN, VEE, M1), flavivirus (e.g., Kunjin, West Nile, Dengue virus), rhabdovirus vectors (e.g., rabies virus, VSV), measles virus vector (e.g., MV-Edm), Newcastle disease virus vectors, poxvirus vectors (e.g., VV), measles virus, and picornavirus vectors (e.g., Coxsackievirus).

In some embodiments, the vector or polycistronic expression cassette comprises one or more additional elements. Additional elements include, but are not limited to, promoters, enhancers, polyadenylation (polyA) sequences, and selection genes.

In some embodiments, the vector comprises a polynucleotide sequence that encodes for a selectable marker that confers a specific trait on cells in which the selectable marker is expressed enabling artificial selection of those cells. Exemplary selectable markers include, but are not limited to, antibiotic resistance genes, e.g., resistance to kanamycin, ampicillin, or triclosan.

In some embodiments, the polycistronic expression cassette comprises a transcriptional regulatory element. Exemplary transcriptional regulatory elements include, but are not limited to promoters and enhancers. In some embodiments, the polycistronic expression cassette comprises a promoter sequence 5′ of the first 5′ cistron. In some embodiments, the promoter comprises a polynucleotide sequence at least 75%, 80%, 85%, 90%, 95%, 96%, 97%, 98%, 99%, or 100% identical to the polynucleotide sequence of SEQ ID NO: 150. In some embodiments, the promoter comprises the polynucleotide sequence of SEQ ID NO: 150. In some embodiments, the polynucleotide sequence of the promoter consists of a polynucleotide sequence at least 75%, 80%, 85%, 90%, 95%, 96%, 97%, 98%, 99%, or 100% identical to the polynucleotide sequence of SEQ ID NO: 150. In some embodiments, the polynucleotide sequence of the promoter consists of the polynucleotide sequence of SEQ ID NO: 150.

In some embodiments, the polycistronic expression cassette comprises a polyA sequence 3′ of the 3′ terminal cistron. In some embodiments, the polyA sequence comprises a polynucleotide sequence at least 75%, 80%, 85%, 90%, 95%, 96%, 97%, 98%, 99%, or 100% identical to the polynucleotide sequence of SEQ ID NO: 151. In some embodiments, the polyA sequence comprises the nucleic acid sequence of SEQ ID NO: 151. In some embodiments, the polyA sequence consists of a sequence at least 75%, 80%, 85%, 90%, 95%, 96%, 97%, 98%, 99%, or 100% identical to the polynucleotide sequence of SEQ ID NO: 151. In some embodiments, the polyA sequence consists of the nucleic acid sequence of SEQ ID NO: 151.

The polynucleotide sequence of exemplary promoters and polyA sequences are provided in Table 9, herein.

TABLE 9
sequences of exemplary promoters and polyA sequences.

| Description | Nucleic Acid Sequence | SEQ ID NO |
|---|---|---|
| hEF-1Rα Hybrid | GGATCTGCGATCGCTCCGGTGCCCGTCAGTGGGCAGAGCGCACATC | 150 |
| Promoter | GCCCACAGTCCCCGAGAAGTTGGGGGGAGGGGTCGGCAATTGAACC | |
| | GGTGCCTAGAGAAGGTGGCGCGGGGTAAACTGGGAAAGTGATGTCG | |
| | TGTACTGGCTCCGCCTTTTTCCCGAGGGTGGGGGAGAACCGTATAT | |
| | AAGTGCAGTAGTCGCCGTGAACGTTCTTTTTCGCAACGGGTTTGCC | |
| | GCCAGAACACAGCTGAAGCTTCGAGGGGCTCGCATCTCTCCTTCAC | |
| | GCGCCCGCCGCCCTACCTGAGGCCGCCATCCACGCCGGTTGAGTCG | |
| | CGTTCTGCCGCCTCCCGCCTGTGGTGCCTCCTGAACTGCGTCCGCC | |
| | GTCTAGGTAAGTTTAAAGCTCAGGTCGAGACCGGGCCTTTGTCCGG | |
| | CGCTCCCTTGGAGCCTACCTAGACTCAGCCGGCTCTCCACGCTTTG | |
| | CCTGACCCTGCTTGCTCAACTCTACGTCTTTGTTTCGTTTTCTGTT | |
| | CTGCGCCGTTACAGATCCAAGCTGTGACCGGCGCCTACCTGAGAT | |
| BGH polyA sequence | GATCTGCTGTGCCTTCTAGTTGCCAGCCATCTGTTGTTTGCCCCTC | 151 |
| | CCCCGTGCCTTCCTTGACCCTGGAAGGTGCCACTCCCACTGTCCTT | |
| | TCCTAATAAAATGAGGAAATTGCATCGCATTGTCTGAGTAGGTGTC | |
| | ATTCTATTCTGGGGGGTGGGGTGGGGCAGGACAGCAAGGGGGAGGA | |
| | TTGGGAAGACAATAGCAGGCATGCTGGGGATGCGGTGGGCTCTATG | |
| | G | |

In some embodiments, the polycistronic expression cassette comprises a polynucleotide sequence that encodes an amino acid sequence at least 90%, 95%, 96%, 97%, 98%, 99% or 100% identical to an amino acid sequence recited in Tables 10A-10C.

TABLE 10A
Exemplary amino acid sequences encoded by polycistronic expression cassettes.

| | | SEQ |
|---|---|---|
| Description | Sequence | ID NO: |
| Cα (murine, | XIQNPEPAVYQLKDPRSQDSTLCLFTDFDSQINVPKTMESGTFITDKXVLDM | 160 |
| degenerate)-fP2A | KAMDSKSNGAIAWSNQTSFTCQDIFKETNATYPSSDVPCDATLTEKSFETDM | |
| | NLNFQNLXVXXLRILLLKVAGFNLLMTLRLWSSRAKRSGSGATNFSLLKQAG | |
| | DVEENPGP | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cβ (murine, | EDLRNVTPPKVSLFEPSKAEIANKQKATLVCLARGFFPDHVELSWWVNGKEV | 161 |
| degenerate)-fT2A- | HSGVXTDPQAYKESNYSYCLSSRLRVSATFWHNPRNHFRCQVQFHGLSEEDK | |
| mbIL15 | WPEGSPKPVTQNISAEAWGRADCGITSASYQQGVLSATILYEILLGKATLYA | |
| | VLVSTLVVMAMVKRKNSRAKRSGSGEGRGSLLTCGDVEENPGPMDWTWILFL | |
| | VAAATRVHSNWVNVISDLKKIEDLIQSMHIDATLYTESDVHPSCKVTAMKCF | |
| | LLELQVISLESGDASIHDTVENLIILANNSLSSNGNVTESGCKECEELEEKN | |
| | IKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTP | |
| | SLKCIRDPALVHQRPAPPSTVTTAGVTPQPESLSPSGKEPAASSPSSNNTAA | |
| | TTAAIVPGSQLMPSKSPSTGTTEISSHESSHGTPSQTTAKNWELTASASHQP | |
| | PGVYPQGHSDTTVAISTSTVLLCGLSAVSLLACYLKSRQTPPLASVEMEAME | |
| | ALPVTWGTSSRDEDLENCSHHL | |
| | X at position 57 is Ser or Cys | |
| Cα (murine, | XIQNPEPAVYQLKDPRSQDSTLCLFTDFDSQINVPKTMESGTFITDKXVLDM | 162 |
| degenerate)-fT2A | KAMDSKSNGAIAWSNQTSFTCQDIFKETNATYPSSDVPCDATLTEKSFETDM | |
| | NLNFQNLXVXXLRILLLKVAGFNLLMTLRLWSSRAKRSGSGEGRGSLLTCGD | |
| | VEENPGP | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cβ (murine, | EDLRNVTPPKVSLFEPSKAEIANKQKATLVCLARGFFPDHVELSWWVNGKEV | 163 |
| degenerate)-fP2A- | HSGVXTDPQAYKESNYSYCLSSRLRVSATFWHNPRNHFRCQVQFHGLSEEDK | |
| mbIL15 | WPEGSPKPVTQNISAEAWGRADCGITSASYQQGVLSATILYEILLGKATLYA | |
| | VLVSTLVVMAMVKRKNSRAKRSGSGATNFSLLKQAGDVEENPGPMDWTWILF | |
| | LVAAATRVHSNWVNVISDLKKIEDLIQSMHIDATLYTESDVHPSCKVTAMKC | |
| | FLLELQVISLESGDASIHDTVENLIILANNSLSSNGNVTESGCKECEELEEK | |
| | NIKEFLQSFVHIVQMFINTSSGGGSGGGGSGGGGSGGGGSGGGSLQITCPPP | |
| | MSVEHADIWVKSYSLYSRERYICNSGFKRKAGTSSLTECVLNKATNVAHWTT | |
| | PSLKCIRDPALVHQRPAPPSTVTTAGVTPQPESLSPSGKEPAASSPSSNNTA | |
| | ATTAAIVPGSQLMPSKSPSTGTTEISSHESSHGTPSQTTAKNWELTASASHQ | |
| | PPGVYPQGHSDTTVAISTSTVLLCGLSAVSLLACYLKSRQTPPLASVEMEAM | |
| | EALPVTWGTSSRDEDLENCSHHL | |
| | X at position 57 is Ser or Cys | |
| Cα (murine, | XIQNPEPAVYQLKDPRSQDSTLCLFTDFDSQINVPKTMESGTFITDKXVLDM | 164 |
| degenerate)-fP2A- | KAMDSKSNGAIAWSNQTSFTCQDIFKETNATYPSSDVPCDATLTEKSFETDM | |
| mbIL15-fT2A | NLNFQNLXVXXLRILLLKVAGFNLLMTLRLWSSRAKRSGSGATNFSLLKQAG | |
| | DVEENPGPMDWTWILFLVAAATRVHSNWVNVISDLKKIEDLIQSMHIDATLY | |
| | TESDVHPSCKVTAMKCFLLELQVISLESGDASIHDTVENLIILANNSLSSNG | |
| | NVTESGCKECEELEEKNIKEFLQSFVHIVQMFINTSSGGGSGGGGSGGGGSG | |
| | GGGSGGGSLQITCPPPMSVEHADIWVKSYSLYSRERYICNSGFKRKAGTSSL | |
| | TECVLNKATNVAHWTTPSLKCIRDPALVHQRPAPPSTVTTAGVTPQPESLSP | |
| | SGKEPAASSPSSNNTAATTAAIVPGSQLMPSKSPSTGTTEISSHESSHGTPS | |
| | QTTAKNWELTASASHQPPGVYPQGHSDTTVAISTSTVLLCGLSAVSLLACYL | |
| | KSRQTPPLASVEMEAMEALPVTWGTSSRDEDLENCSHHLRAKRSGSGEGRGS | |
| | LLTCGDVEENPGP | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cα (murine, | XIQNPEPAVYQLKDPRSQDSTLCLFTDFDSQINVPKTMESGTFITDKXVLDM | 165 |
| degenerate)-fT2A- | KAMDSKSNGAIAWSNQTSFTCQDIFKETNATYPSSDVPCDATLTEKSFETDM | |
| mbIL15-fP2A | NLNFQNLXVXXLRILLLKVAGFNLLMTLRLWSSRAKRSGSGEGRGSLLTCGD | |
| | VEENPGPMDWTWILFLVAAATRVHSNWVNVISDLKKIEDLIQSMHIDATLYT | |
| | ESDVHPSCKVTAMKCFLLELQVISLESGDASIHDTVENLIILANNSLSSNGN | |
| | VTESGCKECEELEEKNIKEFLQSFVHIVQMFINTSSGGGSGGGGSGGGGSGG | |
| | GGSGGGSLQITCPPPMSVEHADIWVKSYSLYSRERYICNSGFKRKAGTSSLT | |
| | ECVLNKATNVAHWTTPSLKCIRDPALVHQRPAPPSTVTTAGVTPQPESLSPS | |
| | GKEPAASSPSSNNTAATTAAIVPGSQLMPSKSPSTGTTEISSHESSHGTPSQ | |
| | TTAKNWELTASASHQPPGVYPQGHSDTTVAISTSTVLLCGLSAVSLLACYLK | |
| | SRQTPPLASVEMEAMEALPVTWGTSSRDEDLENCSHHLRAKRSGSGATNFSL | |
| | LKQAGDVEENPGP | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cβ (murine, | EDLRNVTPPKVSLFEPSKAEIANKQKATLVCLARGFFPDHVELSWWVNGKEV | 166 |
| degenerate)-fP2A | HSGVXTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNSRAKRSGSGATNFSLLKQAGDVEENPGP | |
| | X at position 57 is Ser or Cys | |
| Cα (murine, | XIQNPEPAVYQLKDPRSQDSTLCLFTDFDSQINVPKTMESGTFITDKXVLDM | 167 |
| degenerate)-fT2A- | KAMDSKSNGAIAWSNQTSFTCQDIFKETNATYPSSDVPCDATLTEKSFETDM | |
| mbIL15 | NLNFQNLXVXXLRILLLKVAGFNLLMTLRLWSSRAKRSGSGEGRGSLLTCGD | |
| | VEENPGPMDWTWILFLVAAATRVHSNWVNVISDLKKIEDLIQSMHIDATLYT | |
| | ESDVHPSCKVTAMKCFLLELQVISLESGDASIHDTVENLIILANNSLSSNGN | |
| | VTESGCKECEELEEKNIKEFLQSFVHIVQMFINTSSGGGSGGGGSGGGGSGG | |
| | GGSGGGSLQITCPPPMSVEHADIWVKSYSLYSRERYICNSGFKRKAGTSSLT | |
| | ECVLNKATNVAHWTTPSLKCIRDPALVHQRPAPPSTVTTAGVTPQPESLSPS | |
| | GKEPAASSPSSNNTAATTAAIVPGSQLMPSKSPSTGTTEISSHESSHGTPSQ | |
| | TTAKNWELTASASHQPPGVYPQGHSDTTVAISTSTVLLCGLSAVSLLACYLK | |
| | SRQTPPLASVEMEAMEALPVTWGTSSRDEDLENCSHHL | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cβ (murine, | EDLRNVTPPKVSLFEPSKAEIANKQKATLVCLARGFFPDHVELSWWVNGKEV | 168 |
| degenerate)-fT2A | HSGVXTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNSRAKRSGSGEGRGSLLTCGDVEENPGP | |
| | X at position 57 is Ser or Cys | |
| Cα (murine, | XIQNPEPAVYQLKDPRSQDSTLCLFTDFDSQINVPKTMESGTFITDKXVLDM | 169 |
| degenerate)-fP2A- | KAMDSKSNGAIAWSNQTSFTCQDIFKETNATYPSSDVPCDATLTEKSFETDM | |
| mbIL15 | NLNFQNLXVXXLRILLLKVAGFNLLMTLRLWSSRAKRSGSGATNFSLLKQAG | |
| | DVEENPGPMDWTWILFLVAAATRVHSNWVNVISDLKKIEDLIQSMHIDATLY | |
| | TESDVHPSCKVTAMKCFLLELQVISLESGDASIHDTVENLIILANNSLSSNG | |
| | NVTESGCKECEELEEKNIKEFLQSFVHIVQMFINTSSGGGSGGGGSGGGGSG | |
| | GGGSGGGSLQITCPPPMSVEHADIWVKSYSLYSRERYICNSGFKRKAGTSSL | |
| | TECVLNKATNVAHWTTPSLKCIRDPALVHQRPAPPSTVTTAGVTPQPESLSP | |
| | SGKEPAASSPSSNNTAATTAAIVPGSQLMPSKSPSTGTTEISSHESSHGTPS | |
| | QTTAKNWELTASASHQPPGVYPQGHSDTTVAISTSTVLLCGLSAVSLLACYL | |
| | KSRQTPPLASVEMEAMEALPVTWGTSSRDEDLENCSHHL | |
| | X at position 1 is Asn, Asp, His, or Tyr; | |
| | X at position 48 is Thr or Cys; | |
| | X at position 112 is Ser, Ala, Val, Leu, Ile, Pro, Phe, Met, or Trp; | |
| | X at position 114 is Met, Ala, Val, Leu, Ile, Pro, Phe, or Trp; | |
| | X at position 115 is Gly, Ala, Val, Leu, Ile, Pro, Phe, Met, or Trp | |
| Cβ (murine, | EDLRNVTPPKVSLFEPSKAEIANKQKATLVCLARGFFPDHVELSWWVNGKEV | 170 |
| degenerate)-fP2A- | HSGVXTDPQAYKESNYSYCLSSRLRVSATFWHNPRNHFRCQVQFHGLSEEDK | |
| mbIL15-fT2A | WPEGSPKPVTQNISAEAWGRADCGITSASYQQGVLSATILYEILLGKATLYA | |
| | VLVSTLVVMAMVKRKNSRAKRSGSGATNFSLLKQAGDVEENPGPMDWTWILF | |
| | LVAAATRVHSNWVNVISDLKKIEDLIQSMHIDATLYTESDVHPSCKVTAMKC | |
| | FLLELQVISLESGDASIHDTVENLIILANNSLSSNGNVTESGCKECEELEEK | |
| | NIKEFLQSFVHIVQMFINTSSGGGSGGGGSGGGGSGGGGSGGGSLQITCPPP | |
| | MSVEHADIWVKSYSLYSRERYICNSGFKRKAGTSSLTECVLNKATNVAHWTT | |
| | PSLKCIRDPALVHQRPAPPSTVTTAGVTPQPESLSPSGKEPAASSPSSNNTA | |
| | ATTAAIVPGSQLMPSKSPSTGTTEISSHESSHGTPSQTTAKNWELTASASHQ | |
| | PPGVYPQGHSDTTVAISTSTVLLCGLSAVSLLACYLKSRQTPPLASVEMEAM | |
| | EALPVTWGTSSRDEDLENCSHHLRAKRSGSGEGRGSLLTCGDVEENPGP | |
| | X at position 57 is Ser or Cys | |
| Cβ (murine, | EDLRNVTPPKVSLFEPSKAEIANKQKATLVCLARGFFPDHVELSWWVNGKEV | 171 |
| degenerate)-fT2A- | HSGVXTDPQAYKESNYSYCLSSRLRVSATFWHNPRNHFRCQVQFHGLSEEDK | |
| mbIL15-fP2A | WPEGSPKPVTQNISAEAWGRADCGITSASYQQGVLSATILYEILLGKATLYA | |
| | VLVSTLVVMAMVKRKNSRAKRSGSGEGRGSLLTCGDVEENPGPMDWTWILFL | |
| | VAAATRVHSNWVNVISDLKKIEDLIQSMHIDATLYTESDVHPSCKVTAMKCF | |
| | LLELQVISLESGDASIHDTVENLIILANNSLSSNGNVTESGCKECEELEEKN | |
| | IKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTP | |
| | SLKCIRDPALVHQRPAPPSTVTTAGVTPQPESLSPSGKEPAASSPSSNNTAA | |
| | TTAAIVPGSQLMPSKSPSTGTTEISSHESSHGTPSQTTAKNWELTASASHQP | |
| | PGVYPQGHSDTTVAISTSTVLLCGLSAVSLLACYLKSRQTPPLASVEMEAME | |
| | ALPVTWGTSSRDEDLENCSHHLRAKRSGSGATNFSLLKQAGDVEENPGP | |
| | X at position 57 is Ser or Cys | |
| mbIL15-fP2A | MDWTWILFLVAAATRVHSNWVNVISDLKKIEDLIQSMHIDATLYTESDVHPS | 172 |
| | CKVTAMKCFLLELQVISLESGDASIHDTVENLIILANNSLSSNGNVTESGCK | |
| | ECEELEEKNIKEFLQSFVHIVQMFINTSSGGGSGGGGSGGGGSGGGGSGGGS | |
| | LQITCPPPMSVEHADIWVKSYSLYSRERYICNSGFKRKAGTSSLTECVLNKA | |
| | TNVAHWTTPSLKCIRDPALVHQRPAPPSTVTTAGVTPQPESLSPSGKEPAAS | |
| | SPSSNNTAATTAAIVPGSQLMPSKSPSTGTTEISSHESSHGTPSQTTAKNWE | |
| | LTASASHQPPGVYPQGHSDTTVAISTSTVLLCGLSAVSLLACYLKSRQTPPL | |
| | ASVEMEAMEALPVTWGTSSRDEDLENCSHHLRAKRSGSGATNFSLLKQAGDV | |
| | EENPGP | |
| mbIL15-fT2A | MDWTWILFLVAAATRVHSNWVNVISDLKKIEDLIQSMHIDATLYTESDVHPS | 173 |
| | CKVTAMKCFLLELQVISLESGDASIHDTVENLIILANNSLSSNGNVTESGCK | |
| | ECEELEEKNIKEFLQSFVHIVQMFINTSSGGGSGGGGSGGGGSGGGGSGGGS | |
| | LQITCPPPMSVEHADIWVKSYSLYSRERYICNSGFKRKAGTSSLTECVLNKA | |
| | TNVAHWTTPSLKCIRDPALVHQRPAPPSTVTTAGVTPQPESLSPSGKEPAAS | |
| | SPSSNNTAATTAAIVPGSQLMPSKSPSTGTTEISSHESSHGTPSQTTAKNWE | |
| | LTASASHQPPGVYPQGHSDTTVAISTSTVLLCGLSAVSLLACYLKSRQTPPL | |
| | ASVEMEAMEALPVTWGTSSRDEDLENCSHHLRAKRSGSGEGRGSLLTCGDVE | |
| | ENPGP | |

TABLE 10B
Exemplary amino acid sequences encoded by polycistronic expression cassettes

| | | SEQ |
|---|---|---|
| Description | Sequence | ID NO: |
| Cα (murine, cysteine- | NIQNPEPAVYQLKDPRSQDSTLCLFTDFDSQINVPKTMESGTFITDKCVLDM | 180 |
| and LIV-substituted)- | KAMDSKSNGAIAWSNQTSFTCQDIFKETNATYPSSDVPCDATLTEKSFETDM | |
| fP2A | NLNFQNLLVIVLRILLLKVAGFNLLMTLRLWSSRAKRSGSGATNFSLLKQAG | |
| | DVEENPGP | |
| Cβ (murine, cysteine- | EDLRNVTPPKVSLFEPSKAEIANKQKATLVCLARGFFPDHVELSWWVNGKEV | 181 |
| substituted)-fT2A- | HSGVCTDPQAYKESNYSYCLSSRLRVSATFWHNPRNHFRCQVQFHGLSEEDK | |
| mbIL15 | WPEGSPKPVTQNISAEAWGRADCGITSASYQQGVLSATILYEILLGKATLYA | |
| | VLVSTLVVMAMVKRKNSRAKRSGSGEGRGSLLTCGDVEENPGPMDWTWILFL | |
| | VAAATRVHSNWVNVISDLKKIEDLIQSMHIDATLYTESDVHPSCKVTAMKCF | |
| | LLELQVISLESGDASIHDTVENLIILANNSLSSNGNVTESGCKECEELEEKN | |
| | IKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTP | |
| | SLKCIRDPALVHQRPAPPSTVTTAGVTPQPESLSPSGKEPAASSPSSNNTAA | |
| | TTAAIVPGSQLMPSKSPSTGTTEISSHESSHGTPSQTTAKNWELTASASHQP | |
| | PGVYPQGHSDTTVAISTSTVLLCGLSAVSLLACYLKSRQTPPLASVEMEAME | |
| | ALPVTWGTSSRDEDLENCSHHL | |
| Cα (murine, cysteine- | NIQNPEPAVYQLKDPRSQDSTLCLFTDFDSQINVPKTMESGTFITDKCVLDM | 182 |
| and LIV-substituted)- | KAMDSKSNGAIAWSNQTSFTCQDIFKETNATYPSSDVPCDATLTEKSFETDM | |
| fT2A | NLNFQNLLVIVLRILLLKVAGFNLLMTLRLWSSRAKRSGSGEGRGSLLTCGD | |
| | VEENPGP | |
| Cβ (murine, cysteine- | EDLRNVTPPKVSLFEPSKAEIANKQKATLVCLARGFFPDHVELSWWVNGKEV | 183 |
| substituted)-fP2A- | HSGVCTDPQAYKESNYSYCLSSRLRVSATFWHNPRNHFRCQVQFHGLSEEDK | |
| mbIL15 | WPEGSPKPVTQNISAEAWGRADCGITSASYQQGVLSATILYEILLGKATLYA | |
| | VLVSTLVVMAMVKRKNSRAKRSGSGATNFSLLKQAGDVEENPGPMDWTWILF | |
| | LVAAATRVHSNWVNVISDLKKIEDLIQSMHIDATLYTESDVHPSCKVTAMKC | |
| | FLLELQVISLESGDASIHDTVENLIILANNSLSSNGNVTESGCKECEELEEK | |
| | NIKEFLQSFVHIVQMFINTSSGGGSGGGGSGGGGSGGGGSGGGSLQITCPPP | |
| | MSVEHADIWVKSYSLYSRERYICNSGFKRKAGTSSLTECVLNKATNVAHWTT | |
| | PSLKCIRDPALVHQRPAPPSTVTTAGVTPQPESLSPSGKEPAASSPSSNNTA | |
| | ATTAAIVPGSQLMPSKSPSTGTTEISSHESSHGTPSQTTAKNWELTASASHQ | |
| | PPGVYPQGHSDTTVAISTSTVLLCGLSAVSLLACYLKSRQTPPLASVEMEAM | |
| | EALPVTWGTSSRDEDLENCSHHL | |
| Cα (murine, cysteine- | NIQNPEPAVYQLKDPRSQDSTLCLFTDFDSQINVPKTMESGTFITDKCVLDM | 184 |
| and LIV-substituted)- | KAMDSKSNGAIAWSNQTSFTCQDIFKETNATYPSSDVPCDATLTEKSFETDM | |
| fP2A-mbIL15-fT2A | NLNFQNLLVIVLRILLLKVAGFNLLMTLRLWSSRAKRSGSGATNFSLLKQAG | |
| | DVEENPGPMDWTWILFLVAAATRVHSNWVNVISDLKKIEDLIQSMHIDATLY | |
| | TESDVHPSCKVTAMKCFLLELQVISLESGDASIHDTVENLIILANNSLSSNG | |
| | NVTESGCKECEELEEKNIKEFLQSGVHIVQMFINTSSGGGSGGGGSGGGGSG | |
| | GGGSGGGSLQITCPPPMSVEHADIWVKSYSLYSRERYICNSGFKRKAGTSSL | |
| | TECVLNKATNVAHWTTPSLKCIRDPALVHQRPAPPSTVTTAGVTPQPESLSP | |
| | SGKEPAASSPSSNNTAATTAAIVPGSQLMPSKSPSTGTTEISSHESSHGTPS | |
| | QTTAKNWELTASASHQPPGVYPQGHSDTTVAISTSTVLLCGLSAVSLLACYL | |
| | KSRQTPPLASVEMEAMEALPVTWGTSSRDEDLENCSHHLRAKRSGSGEGRGS | |
| | LLTCGDVEENPGP | |
| Cα (murine, cysteine- | NIQNPEPAVYQLKDPRSQDSTLCLFTDFDSQINVPKTMESGTFITDKCVLDM | 185 |
| and LIV-substituted)- | KAMDSKSNGAIAWSNQTSFTCQDIFKETNATYPSSDVPCDATLTEKSFETDM | |
| fT2A-mbIL15-fP2A | NLNFQNLLVIVLRILLLKVAGFNLLMTLRLWSSRAKRSGSGEGRGSLLTCGD | |
| | VEENPGPMDWTWILFLVAAATRVHSNWVNVISDLKKIEDLIQSMHIDATLYT | |
| | ESDVHPSCKVTAMKCFLLELQVISLESGDASIHDTVENLIILANNSLSSNGN | |
| | VTESGCKECEELEEKNIKEFLQSFVHIVQMFINTSSGGGSGGGGSGGGGSGG | |
| | GGSGGGSLQITCPPPMSVEHADIWVKSYSLYSRERYICNSGFKRKAGTSSLT | |
| | ECVLNKATNVAHWTTPSLKCIRDPALVHQRPAPPSTVTTAGVTPQPESLSPS | |
| | GKEPAASSPSSNNTAATTAAIVPGSQLMPSKSPSTGTTEISSHESSHGTPSQ | |
| | TTAKNWELTASASHQPPGVYPQGHSDTTVAISTSTVLLCGLSAVSLLACYLK | |
| | SRQTPPLASVEMEAMEALPVTWGTSSRDEDLENCSHHLRAKRSGSGATNFSL | |
| | LKQAGDVEENPGP | |
| Cβ (murine, cysteine- | EDLRNVTPPKVSLFEPSKAEIANKQKATLVCLARGFFPDHVELSWWVNGKEV | 186 |
| substituted)-fP2A | HSGVCTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNSRAKRSGSGATNFSLLKQAGDVEENPGP | |
| Cα (murine, cysteine- | NIQNPEPAVYQLKDPRSQDSTLCLFTDFDSQINVPKTMESGTFITDKCVLDM | 187 |
| and LIV-substituted)- | KAMDSKSNGAIAWSNQTSFTCQDIFKETNATYPSSDVPCDATLTEKSFETDM | |
| fT2A-mbIL15 | NLNFQNLLVIVLRILLLKVAGFNLLMTLRLWSSRAKRSGSGEGRGSLLTCGD | |
| | VEENPGPMDWTWILFLVAAATRVHSNWVNVISDLKKIEDLIQSMHIDATLYT | |
| | ESDVHPSCKVTAMKCFLLELQVISLESGDASIHDTVENLIILANNSLSSNGN | |
| | VTESGCKECEELEEKNIKEFLQSFVHIVQMFINTSSGGGSGGGGSGGGGSGG | |
| | GGSGGGSLQITCPPPMSVEHADIWVKSYSLYSRERYICNSGFKRKAGTSSLT | |
| | ECVLNKATNVAHWTTPSLKCIRDPALVHQRPAPPSTVTTAGVTPQPESLSPS | |
| | GKEPAASSPSSNNTAATTAAIVPGSQLMPSKSPSTGTTEISSHESSHGTPSQ | |
| | TTAKNWELTASASHQPPGVYPQGHSDTTVAISTSTVLLCGLSAVSLLACYLK | |
| | SRQTPPLASVEMEAMEALPVTWGTSSRDEDLENCSHHL | |
| Cβ (murine, cysteine- | EDLRNVTPPKVSLFEPSKAEIANKQKATLVCLARGFFPDHVELSWWVNGKEV | 188 |
| substituted)-fT2A | HSGVCTDPQAYKESNYSYCLSSRLRVSATFWHNPRNHFRCQVQFHGLSEEDK | |
| | WPEGSPKPVTQNISAEAWGRADCGITSASYQQGVLSATILYEILLGKATLYA | |
| | VLVSTLVVMAMVKRKNSRAKRSGSGEGRGSLLTCGDVEENPGP | |
| Cα (murine, cysteine- | NIQNPEPAVYQLKDPRSQDSTLCLFTDFDSQINVPKTMESGTFITDKCVLDM | 189 |
| and LIV-substituted)- | KAMDSKSNGAIAWSNQTSFTCQDIFKETNATYPSSDVPCDATLTEKSFETDM | |
| fP2A-mbIL15 | NLNFQNLLVIVLRILLLKVAGFNLLMTLRLWSSRAKRSGSGATNFSLLKQAG | |
| | DVEENPGPMDWTWILFLVAAATRVHSNWVNVISDLKKIEDLIQSMHIDATLY | |
| | TESDVHPSCKVTAMKCFLLELQVISLESGDASIHDTVENLIILANNSLSSNG | |
| | NVTESGCKECEELEEKNIKEFLQSGVHIVQMFINTSSGGGSGGGGSGGGGSG | |
| | GGGSGGGSLQITCPPPMSVEHADIWVKSYSLYSRERYICNSGFKRKAGTSSL | |
| | TECVLNKATNVAHWTTPSLKCIRDPALVHQRPAPPSTVTTAGVTPQPESLSP | |
| | SGKEPAASSPSSNNTAATTAAIVPGSQLMPSKSPSTGTTEISSHESSHGTPS | |
| | QTTAKNWELTASASHQPPGVYPQGHSDTTVAISTSTVLLCGLSAVSLLACYL | |
| | KSRQTPPLASVEMEAMEALPVTWGTSSRDEDLENCSHHL | |
| Cβ (murine, cysteine- | EDLRNVTPPKVSLFEPSKAEIANKQKATLVCLARGFFPDHVELSWWVNGKEV | 190 |
| substituted)-fP2A- | HSGVCTDPQAYKESNYSYCLSSRLRVSATFWHNPRNHFRCQVQFHGLSEEDK | |
| mbIL15-fT2A | WPEGSPKPVTQNISAEAWGRADCGITSASYQQGVLSATILYEILLGKATLYA | |
| | VLVSTLVVMAMVKRKNSRAKRSGSGATNFSLLKQAGDVEENPGPMDWTWILF | |
| | LVAAATRVHSNWVNVISDLKKIEDLIQSMHIDATLYTESDVHPSCKVTAMKC | |
| | FLLELQVISLESGDASIHDTVENLIILANNSLSSNGNVTESGCKECEELEEK | |
| | NIKEFLQSFVHIVQMFINTSSGGGSGGGGSGGGGSGGGGSGGGSLQITCPPP | |
| | MSVEHADIWVKSYSLYSRERYICNSGFKRKAGTSSLTECVLNKATNVAHWTT | |
| | PSLKCIRDPALVHQRPAPPSTVTTAGVTPQPESLSPSGKEPAASSPSSNNTA | |
| | ATTAAIVPGSQLMPSKSPSTGTTEISSHESSHGTPSQTTAKNWELTASASHQ | |
| | PPGVYPQGHSDTTVAISTSTVLLCGLSAVSLLACYLKSRQTPPLASVEMEAM | |
| | EALPVTWGTSSRDEDLENCSHHLRAKRSGSGEGRGSLLTCGDVEENPGP | |
| Cβ (murine, cysteine- | EDLRNVTPPKVSLFEPSKAEIANKQKATLVCLARGFFPDHVELSWWVNGKEV | 191 |
| substituted)-fT2A- | HSGVCTDPQAYKESNYSYCLSSRLRVSATFWHNPRNHFRCQVQFHGLSEEDK | |
| mbIL15-fP2A | WPEGSPKPVTQNISAEAWGRADCGITSASYQQGVLSATILYEILLGKATLYA | |
| | VLVSTLVVMAMVKRKNSRAKRSGSGEGRGSLLTCGDVEENPGPMDWTWILFL | |
| | VAAATRVHSNWVNVISDLKKIEDLIQSMHIDATLYTESDVHPSCKVTAMKCF | |
| | LLELQVISLESGDASIHDTVENLIILANNSLSSNGNVTESGCKECEELEEKN | |
| | IKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTP | |
| | SLKCIRDPALVHQRPAPPSTVTTAGVTPQPESLSPSGKEPAASSPSSNNTAA | |
| | TTAAIVPGSQLMPSKSPSTGTTEISSHESSHGTPSQTTAKNWELTASASHQP | |
| | PGVYPQGHSDTTVAISTSTVLLCGLSAVSLLACYLKSRQTPPLASVEMEAME | |
| | ALPVTWGTSSRDEDLENCSHHLRAKRSGSGATNFSLLKQAGDVEENPGP | |

TABLE 10C
Exemplary amino acid sequences encoded by polycistronic expression cassettes

| | | SEQ |
|---|---|---|
| Description | Sequence | ID NO: |
| Cα (murine, LIV | NIQNPEPAVYQLKDPRSQDSTLCLFTDFDSQINVPKTMESGTFITDKTVLDM | 210 |
| substituted)-fP2A | KAMDSKSNGAIAWSNQTSFTCQDIFKETNATYPSSDVPCDATLTEKSFETDM | |
| | NLNFQNLLVIVLRILLLKVAGFNLLMTLRLWSSRAKRSGSGATNFSLLKQAG | |
| | DVEENPGP | |
| Cα (murine, LIV | NIQNPEPAVYQLKDPRSQDSTLCLFTDFDSQINVPKTMESGTFITDKTVLDM | 212 |
| substituted)-fT2A | KAMDSKSNGAIAWSNQTSFTCQDIFKETNATYPSSDVPCDATLTEKSFETDM | |
| | NLNFQNLLVIVLRILLLKVAGFNLLMTLRLWSSRAKRSGSGEGRGSLLTCGD | |
| | VEENPGP | |
| Cα (murine, LIV | NIQNPEPAVYQLKDPRSQDSTLCLFTDFDSQINVPKTMESGTFITDKTVLDM | 214 |
| substituted)-fP2A- | KAMDSKSNGAIAWSNQTSFTCQDIFKETNATYPSSDVPCDATLTEKSFETDM | |
| mbIL15-fT2A | NLNFQNLLVIVLRILLLKVAGFNLLMTLRLWSSRAKRSGSGATNFSLLKQAG | |
| | DVEENPGPMDWTWILFLVAAATRVHSNWVNVISDLKKIEDLIQSMHIDATLY | |
| | TESDVHPSCKVTAMKCFLLELQVISLESGDASIHDTVENLIILANNSLSSNG | |
| | NVTESGCKECEELEEKNIKEFLQSFVHIVQMFINTSSGGGSGGGGSGGGGSG | |
| | GGGSGGGSLQITCPPPMSVEHADIWVKSYSLYSRERYICNSGFKRKAGTSSL | |
| | TECVLNKATNVAHWTTPSLKCIRDPALVHQRPAPPSTVTTAGVTPQPESLSP | |
| | SGKEPAASSPSSNNTAATTAAIVPGSQLMPSKSPSTGTTEISSHESSHGTPS | |
| | QTTAKNWELTASASHQPPGVYPQGHSDTTVAISTSTVLLCGLSAVSLLACYL | |
| | KSRQTPPLASVEMEAMEALPVTWGTSSRDEDLENCSHHLRAKRSGSGEGRGS | |
| | LLTCGDVEENPGP | |
| Cα (murine, LIV | NIQNPEPAVYQLKDPRSQDSTLCLFTDFDSQINVPKTMESGTFITDKTVLDM | 215 |
| substituted)-fT2A- | KAMDSKSNGAIAWSNQTSFTCQDIFKETNATYPSSDVPCDATLTEKSFETDM | |
| mbIL15-fP2A | NLNFQNLLVIVLRILLLKVAGFNLLMTLRLWSSRAKRSGSGEGRGSLLTCGD | |
| | VEENPGPMDWTWILFLVAAATRVHSNWVNVISDLKKIEDLIQSMHIDATLYT | |
| | ESDVHPSCKVTAMKCFLLELQVISLESGDASIHDTVENLIILANNSLSSNGN | |
| | VTESGCKECEELEEKNIKEFLQSFVHIVQMFINTSSGGGSGGGGSGGGGSGG | |
| | GGSGGGSLQITCPPPMSVEHADIWVKSYSLYSRERYICNSGFKRKAGTSSLT | |
| | ECVLNKATNVAHWTTPSLKCIRDPALVHQRPAPPSTVTTAGVTPQPESLSPS | |
| | GKEPAASSPSSNNTAATTAAIVPGSQLMPSKSPSTGTTEISSHESSHGTPSQ | |
| | TTAKNWELTASASHQPPGVYPQGHSDTTVAISTSTVLLCGLSAVSLLACYLK | |
| | SRQTPPLASVEMEAMEALPVTWGTSSRDEDLENCSHHLRAKRSGSGATNFSL | |
| | LKQAGDVEENPGP | |
| Cα (murine, LIV | NIQNPEPAVYQLKDPRSQDSTLCLFTDFDSQINVPKTMESGTFITDKTVLDM | 217 |
| substituted)-fT2A- | KAMDSKSNGAIAWSNQTSFTCQDIFKETNATYPSSDVPCDATLTEKSFETDM | |
| mbIL15 | NLNFQNLLVIVLRILLLKVAGFNLLMTLRLWSSRAKRSGSGEGRGSLLTCGD | |
| | VEENPGPMDWTWILFLVAAATRVHSNWVNVISDLKKIEDLIQSMHIDATLYT | |
| | ESDVHPSCKVTAMKCFLLELQVISLESGDASIHDTVENLIILANNSLSSNGN | |
| | VTESGCKECEELEEKNIKEFLQSFVHIVQMFINTSSGGGSGGGGSGGGGSGG | |
| | GGSGGGSLQITCPPPMSVEHADIWVKSYSLYSRERYICNSGFKRKAGTSSLT | |
| | ECVLNKATNVAHWTTPSLKCIRDPALVHQRPAPPSTVTTAGVTPQPESLSPS | |
| | GKEPAASSPSSNNTAATTAAIVPGSQLMPSKSPSTGTTEISSHESSHGTPSQ | |
| | TTAKNWELTASASHQPPGVYPQGHSDTTVAISTSTVLLCGLSAVSLLACYLK | |
| | SRQTPPLASVEMEAMEALPVTWGTSSRDEDLENCSHHL | |
| Cα (murine, LIV | NIQNPEPAVYQLKDPRSQDSTLCLFTDFDSQINVPKTMESGTFITDKTVLDM | 219 |
| substituted)-fP2A- | KAMDSKSNGAIAWSNQTSFTCQDIFKETNATYPSSDVPCDATLTEKSFETDM | |
| mbIL15 | NLNFQNLLVIVLRILLLKVAGFNLLMTLRLWSSRAKRSGSGATNFSLLKQAG | |
| | DVEENPGPMDWTWILFLVAAATRVHSNWVNVISDLKKIEDLIQSMHIDATLY | |
| | TESDVHPSCKVTAMKCFLLELQVISLESGDASIHDTVENLIILANNSLSSNG | |
| | NVTESGCKECEELEEKNIKEFLQSFVHIVQMFINTSSGGGSGGGGSGGGGSG | |
| | GGGSGGGSLQITCPPPMSVEHADIWVKSYSLYSRERYICNSGFKRKAGTSSL | |
| | TECVLNKATNVAHWTTPSLKCIRDPALVHQRPAPPSTVTTAGVTPQPESLSP | |
| | SGKEPAASSPSSNNTAATTAAIVPGSQLMPSKSPSTGTTEISSHESSHGTPS | |
| | QTTAKNWELTASASHQPPGVYPQGHSDTTVAISTSTVLLCGLSAVSLLACYL | |
| | KSRQTPPLASVEMEAMEALPVTWGTSSRDEDLENCSHHL | |

Tables 11A, B and C below provide exemplary polynucleotide sequences for use in constructing vectors that may be used in the present disclosure. As shown in Tables 11A, B and C, vectors of the present disclosure can include one or more of the following sequences: (1) an “AP” sequence which encodes (i) a Cα sequence disclosed herein and (ii) a P2A element sequence disclosed herein; (2) a “BT” sequence which encodes (i) a Cβ sequence disclosed herein and (ii) a T2A element sequence disclosed herein; (3) a “BT15” sequence which encodes (i) a Cβ sequence disclosed herein, (ii) a T2A element sequence disclosed herein, and (iii) a mbIL15 sequence disclosed herein; (4) an “AT” sequence which encodes (i) a Cα sequence disclosed herein and (ii) a T2A element sequence disclosed herein; (5) a “BP” sequence which encodes (i) a Cβ sequence disclosed herein and (ii) a P2A element sequence disclosed herein; (6) a “BP15” sequence which encodes (i) a Cβ sequence disclosed herein, (ii) a P2A element sequence disclosed herein, and (iii) a mbIL15 sequence disclosed herein; (7) an “A-P15” sequence which encodes (i) a Cα sequence disclosed herein, (ii) a P2A element sequence disclosed herein, and (iii) a mbIL15 sequence disclosed herein; (8) a “15T” sequence which encodes (i) a mbIL15 sequence disclosed herein and (ii) a T2A element sequence disclosed herein; (9) an “AP15T” sequence which encodes (i) a Cα sequence disclosed herein, (ii) a P2A element sequence disclosed herein, (iii) a mbIL15 sequence disclosed herein, and (iv) a T2A element sequence disclosed herein (10) an “AT15” sequence which encodes (i) a Cα sequence disclosed herein, (ii) a T2A element sequence disclosed herein, and (iii) a mbIL15 sequence disclosed herein; (11) a “15P” sequence which encodes (i) a mbIL15 sequence disclosed herein, and (ii) a P2A element sequence disclosed herein; (12) an “AT 15P” sequence which encodes (i) a Cα sequence disclosed herein, (ii) a T2A element sequence disclosed herein, (iii) a mbIL15 sequence disclosed herein, and (iv) a P2A element sequence disclosed herein; (13) an “BP15T” sequence which encodes (i) a Cβ sequence disclosed herein, (ii) a P2A element sequence disclosed herein, (iii) a mbIL15 sequence disclosed herein, and (iv) a T2A element sequence disclosed herein; (14) an “BT15P” sequence which encodes (i) a Cβ sequence disclosed herein, (ii) a T2A element sequence disclosed herein, (iii) a mbIL15 sequence disclosed herein, and (iv) a P2A element sequence disclosed herein.

The nucleotide sequences provided herein (and their corresponding amino acid sequences) may be used in any appropriate combination. An “appropriate combination” is a combination where desired molecular function(s) are provided by one or more of the sequences disclosed herein. For example, in general, any 2A element sequence provided herein can provide the function of ribosome skipping (via the 2A element) and, optionally, furin-mediated cleavage (via the furin recognition site). Thus, an “AT” sequence in a vector of the present disclosure could, in alternative embodiments, be replaced by an “AP” sequence of the present disclosure. Similarly, “AE” and “AF” sequences, comprising Cα region sequences and E2A or F2A element sequences can also be used. “BT,” “BP,” “BE,” and “BF” sequences comprising Cβ region sequences and 2A element sequences are all also interchangeable. “15T,” “15P,” “15E,” and “15F” sequences comprising mbIL15 sequences and 2A element sequences are all also interchangeable. Additionally, any combination of TCRα, TCRβ, and mbIL15 sequences may appear from 5′ to 3′ on a vector of the present disclosure in any order and may be separated by sequences which provide appropriate 2A element sequence function (e.g., ribosome skipping, furin cleavage).

Accordingly, sequences of the present disclosure provide ribosome skipping, furin recognition, TCRα function, TCRβ function, and mbIL15 function in any appropriate combination or 5′ to 3′ order.

TABLE 11A
Exemplary polynucleotide sequences for use in polycistronic expression cassettes.

| | | SEQ |
|---|---|---|
| Description | Sequence | ID NO: |
| AP nucleotide | NNNATCCAGAATCCCGAGCCTGCGGTGTACCAGCTGAAGGACCCCCGCTCT | 230 |
| sequence | CAGGATAGCACACTGTGCCTGTTCACCGACTTTGATAGCCAGATCAACGTG | |
| | CCTAAAACAATGGAGTCCGGCACCTTCATCACCGACAAGNNNGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGNNNGTCNNNNNNCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCA | |
| | NNN at positions 1-3 make up a codon that encodes Asn, Asp, His, or Tyr; | |
| | NNN at positions 142-144 make up a codon that encodes Thr or Cys; | |
| | NNN at positions 334-336 make up a codon that encodes Ser, Ala, Val, Leu, | |
| | Ile, Pro, Phe, Met, or Trp; | |
| | NNN at positions 340-342 make up a codon that encodes Met, Ala, Val, Leu, | |
| | Ile, Pro, Phe, or Trp; | |
| | NNN at positions 343-345 make up a codon that encodes Gly, Ala, Val, Leu, | |
| | Ile, Pro, Phe, Met, or Trp | |
| BT nucleotide | GAGGACCTGAGGAACGTGACCCCACCTAAAGTGAGCCTGTTCGAGCCATCC | 231 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGNNNACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGAGGGCAGAGGAAGTCTT | |
| | CTAACATGCGGTGACGTGGAGGAGAATCCCGGCCCT | |
| | NNN at positions 169-171 make up a codon that encodes Ser or Cys | |
| BT15 nucleotide | GAGGACCTGAGGAACGTGACCCCACCTAAAGTGAGCCTGTTCGAGCCATCC | 232 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGNNNACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGAGGGCAGAGGAAGTCTT | |
| | CTAACATGCGGTGACGTGGAGGAGAATCCCGGCCCTATGGATTGGACCTGG | |
| | ATTCTGTTTCTGGTGGCCGCTGCCACAAGAGTGCACAGCAACTGGGTGAAT | |
| | GTGATCAGCGACCTGAAGAAGATCGAGGATCTGATCCAGAGCATGCACATT | |
| | GATGCCACCCTGTACACAGAATCTGATGTGCACCCTAGCTGTAAAGTGACC | |
| | GCCATGAAGTGTTTTCTGCTGGAGCTGCAGGTGATTTCTCTGGAAAGCGGA | |
| | GATGCCTCTATCCACGACACAGTGGAGAATCTGATCATCCTGGCCAACAAT | |
| | AGCCTGAGCAGCAATGGCAATGTGACAGAGTCTGGCTGTAAGGAGTGTGAG | |
| | GAGCTGGAGGAGAAGAACATCAAGGAGTTTCTGCAGAGCTTTGTGCACATC | |
| | GTGCAGATGTTCATCAATACAAGCTCTGGCGGAGGATCTGGAGGAGGCGGA | |
| | TCTGGAGGAGGAGGCAGTGGAGGCGGAGGATCTGGCGGAGGATCTCTGCAG | |
| | ATTACATGCCCTCCTCCAATGTCTGTGGAGCACGCCGATATTTGGGTGAAG | |
| | TCCTACAGCCTGTACAGCAGAGAGAGATACATCTGCAACAGCGGCTTTAAG | |
| | AGAAAGGCCGGCACCTCTTCTCTGACAGAGTGCGTGCTGAATAAGGCCACA | |
| | AATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTG | |
| | ACACCTCAGCCTGAATCTCTGAGCCCTTCTGGAAAAGAACCTGCCGCCAGC | |
| | TCTCCTAGCTCTAATAATACCGCCGCCACAACAGCCGCCATTGTGCCTGGA | |
| | TCTCAGCTGATGCCTAGCAAGTCTCCTAGCACAGGCACAACAGAGATCAGC | |
| | AGCCACGAATCTTCTCACGGAACACCTTCTCAGACCACCGCCAAGAATTGG | |
| | GAGCTGACAGCCTCTGCCTCTCACCAGCCTCCAGGAGTGTATCCTCAGGGC | |
| | CACTCTGATACAACAGTGGCCATCAGCACATCTACAGTGCTGCTGTGTGGA | |
| | CTGTCTGCCGTGTCTCTGCTGGCCTGTTACCTGAAGTCTAGACAGACACCT | |
| | CCTCTGGCCTCTGTGGAGATGGAGGCCATGGAAGCCCTGCCTGTGACATGG | |
| | GGAACAAGCAGCAGAGATGAAGACCTGGAGAATTGTTCTCACCACCTG | |
| | NNN at positions 169-171 make up a codon that encodes Ser or Cys | |
| AT nucleotide | NNNATCCAGAATCCCGAGCCTGCGGTGTACCAGCTGAAGGACCCCCGCTCT | 233 |
| sequence | CAGGATAGCACACTGTGCCTGTTCACCGACTTTGATAGCCAGATCAACGTG | |
| | CCTAAAACAATGGAGTCCGGCACCTTCATCACCGACAAGNNNGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGNNNGTCNNNNNNCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCT | |
| | NNN at positions 1-3 make up a codon that encodes Asn, Asp, His, or Tyr; | |
| | NNN at positions 142-144 make up a codon that encodes Thr or Cys; | |
| | NNN at positions 334-336 make up a codon that encodes Ser, Ala, Val, Leu, | |
| | Ile, Pro, Phe, Met, or Trp; | |
| | NNN at positions 340-342 make up a codon that encodes Met, Ala, Val, Leu, | |
| | Ile, Pro, Phe, or Trp; | |
| | NNN at positions 343-345 make up a codon that encodes Gly, Ala, Val, Leu, | |
| | Ile, Pro, Phe, Met, or Trp | |
| BP nucleotide | GAGGACCTGAGGAACGTGACCCCACCTAAAGTGAGCCTGTTCGAGCCATCC | 234 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGNNNACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGCGACCAATTTCAGCCTG | |
| | CTGAAGCAGGCGGGCGATGTGGAGGAGAACCCTGGCCCA | |
| | NNN at positions 169-171 make up a codon that encodes Ser or Cys | |
| BP15 nucleotide | GAGGACCTGAGGAACGTGACCCCACCTAAAGTGAGCCTGTTCGAGCCATCC | 235 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGNNNACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGCGACCAATTTCAGCCTG | |
| | CTGAAGCAGGCGGGCGATGTGGAGGAGAACCCTGGCCCAATGGATTGGACC | |
| | TGGATTCTGTTTCTGGTGGCCGCTGCCACAAGAGTGCACAGCAACTGGGTG | |
| | AATGTGATCAGCGACCTGAAGAAGATCGAGGATCTGATCCAGAGCATGCAC | |
| | ATTGATGCCACCCTGTACACAGAATCTGATGTGCACCCTAGCTGTAAAGTG | |
| | ACCGCCATGAAGTGTTTTCTGCTGGAGCTGCAGGTGATTTCTCTGGAAAGC | |
| | GGAGATGCCTCTATCCACGACACAGTGGAGAATCTGATCATCCTGGCCAAC | |
| | AATAGCCTGAGCAGCAATGGCAATGTGACAGAGTCTGGCTGTAAGGAGTGT | |
| | GAGGAGCTGGAGGAGAAGAACATCAAGGAGTTTCTGCAGAGCTTTGTGCAC | |
| | ATCGTGCAGATGTTCATCAATACAAGCTCTGGCGGAGGATCTGGAGGAGGC | |
| | GGATCTGGAGGAGGAGGCAGTGGAGGCGGAGGATCTGGCGGAGGATCTCTG | |
| | CAGATTACATGCCCTCCTCCAATGTCTGTGGAGCACGCCGATATTTGGGTG | |
| | AAGTCCTACAGCCTGTACAGCAGAGAGAGATACATCTGCAACAGCGGCTTT | |
| | AAGAGAAAGGCCGGCACCTCTTCTCTGACAGAGTGCGTGCTGAATAAGGCC | |
| | ACAAATGTGGCCCACTGGACAACACCTAGCCTGAAGTGCATTAGAGATCCT | |
| | GCCCTGGTCCACCAGAGGCCTGCCCCTCCATCTACAGTGACAACAGCCGGA | |
| | GTGACACCTCAGCCTGAATCTCTGAGCCCTTCTGGAAAAGAACCTGCCGCC | |
| | AGCTCTCCTAGCTCTAATAATACCGCCGCCACAACAGCCGCCATTGTGCCT | |
| | GGATCTCAGCTGATGCCTAGCAAGTCTCCTAGCACAGGCACAACAGAGATC | |
| | AGCAGCCACGAATCTTCTCACGGAACACCTTCTCAGACCACCGCCAAGAAT | |
| | TGGGAGCTGACAGCCTCTGCCTCTCACCAGCCTCCAGGAGTGTATCCTCAG | |
| | GGCCACTCTGATACAACAGTGGCCATCAGCACATCTACAGTGCTGCTGTGT | |
| | GGACTGTCTGCCGTGTCTCTGCTGGCCTGTTACCTGAAGTCTAGACAGACA | |
| | CCTCCTCTGGCCTCTGTGGAGATGGAGGCCATGGAAGCCCTGCCTGTGACA | |
| | TGGGGAACAAGCAGCAGAGATGAAGACCTGGAGAATTGTTCTCACCACCTG | |
| | NNN at positions 169-171 make up a codon that encodes Ser or Cys | |
| AP15 nucleotide | NNNATCCAGAATCCCGAGCCTGCGGTGTACCAGCTGAAGGACCCCCGCTCT | 236 |
| sequence | CAGGATAGCACACTGTGCCTGTTCACCGACTTTGATAGCCAGATCAACGTG | |
| | CCTAAAACAATGGAGTCCGGCACCTTCATCACCGACAAGNNNGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGNNNGTCNNNNNNCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCAATGGATTGGACCTGGATT | |
| | CTGTTTCTGGTGGCCGCTGCCACAAGAGTGCACAGCAACTGGGTGAATGTG | |
| | ATCAGCGACCTGAAGAAGATCGAGGATCTGATCCAGAGCATGCACATTGAT | |
| | GCCACCCTGTACACAGAATCTGATGTGCACCCTAGCTGTAAAGTGACCGCC | |
| | ATGAAGTGTTTTCTGCTGGAGCTGCAGGTGATTTCTCTGGAAAGCGGAGAT | |
| | GCCTCTATCCACGACACAGTGGAGAATCTGATCATCCTGGCCAACAATAGC | |
| | CTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTG | |
| | CAGATGTTCATCAATACAAGCTCTGGCGGAGGATCTGGAGGAGGCGGATCT | |
| | GGAGGAGGAGGCAGTGGAGGCGGAGGATCTGGCGGAGGATCTCTGCAGATT | |
| | ACATGCCCTCCTCCAATGTCTGTGGAGCACGCCGATATTTGGGTGAAGTCC | |
| | TACAGCCTGTACAGCAGAGAGAGATACATCTGCAACAGCGGCTTTAAGAGA | |
| | AAGGCCGGCACCTCTTCTCTGACAGAGTGCGTGCTGAATAAGGCCACAAAT | |
| | GTGGCCCACTGGACAACACCTAGCCTGAAGTGCATTAGAGATCCTGCCCTG | |
| | GTCCACCAGAGGCCTGCCCCTCCATCTACAGTGACAACAGCCGGAGTGACA | |
| | CCTCAGCCTGAATCTCTGAGCCCTTCTGGAAAAGAACCTGCCGCCAGCTCT | |
| | CCTAGCTCTAATAATACCGCCGCCACAACAGCCGCCATTGTGCCTGGATCT | |
| | CAGCTGATGCCTAGCAAGTCTCCTAGCACAGGCACAACAGAGATCAGCAGC | |
| | CACGAATCTTCTCACGGAACACCTTCTCAGACCACCGCCAAGAATTGGGAG | |
| | CTGACAGCCTCTGCCTCTCACCAGCCTCCAGGAGTGTATCCTCAGGGCCAC | |
| | TCTGATACAACAGTGGCCATCAGCACATCTACAGTGCTGCTGTGTGGACTG | |
| | TCTGCCGTGTCTCTGCTGGCCTGTTACCTGAAGTCTAGACAGACACCTCCT | |
| | CTGGCCTCTGTGGAGATGGAGGCCATGGAAGCCCTGCCTGTGACATGGGGA | |
| | ACAAGCAGCAGAGATGAAGACCTGGAGAATTGTTCTCACCACCTG | |
| | NNN at positions 1-3 make up a codon that encodes Asn, Asp, His, or Tyr; | |
| | NNN at positions 142-144 make up a codon that encodes Thr or Cys; | |
| | NNN at positions 334-336 make up a codon that encodes Ser, Ala, Val, Leu, | |
| | Ile, Pro, Phe, Met, or Trp; | |
| | NNN at positions 340-342 make up a codon that encodes Met, Ala, Val, Leu, | |
| | Ile, Pro, Phe, or Trp; | |
| | NNN at positions 343-345 make up a codon that encodes Gly, Ala, Val, Leu, | |
| | Ile, Pro, Phe, Met, or Trp | |
| 15T nucleotide | ATGGATTGGACCTGGATTCTGTTTCTGGTGGCCGCTGCCACAAGAGTGCAC | 237 |
| sequence | AGCAACTGGGTGAATGTGATCAGCGACCTGAAGAAGATCGAGGATCTGATC | |
| | CAGAGCATGCACATTGATGCCACCCTGTACACAGAATCTGATGTGCACCCT | |
| | AGCTGTAAAGTGACCGCCATGAAGTGTTTTCTGCTGGAGCTGCAGGTGATT | |
| | TCTCTGGAAAGCGGAGATGCCTCTATCCACGACACAGTGGAGAATCTGATC | |
| | ATCCTGGCCAACAATAGCCTGAGCAGCAATGGCAATGTGACAGAGTCTGGC | |
| | TGTAAGGAGTGTGAGGAGCTGGAGGAGAAGAACATCAAGGAGTTTCTGCAG | |
| | AGCTTTGTGCACATCGTGCAGATGTTCATCAATACAAGCTCTGGCGGAGGA | |
| | TCTGGAGGAGGCGGATCTGGAGGAGGAGGCAGTGGAGGCGGAGGATCTGGC | |
| | GGAGGATCTCTGCAGATTACATGCCCTCCTCCAATGTCTGTGGAGCACGCC | |
| | GATATTTGGGTGAAGTCCTACAGCCTGTACAGCAGAGAGAGATACATCTGC | |
| | AACAGCGGCTTTAAGAGAAAGGCCGGCACCTCTTCTCTGACAGAGTGCGTG | |
| | CTGAATAAGGCCACAAATGTGGCCCACTGGACAACACCTAGCCTGAAGTGC | |
| | ATTAGAGATCCTGCCCTGGTCCACCAGAGGCCTGCCCCTCCATCTACAGTG | |
| | ACAACAGCCGGAGTGACACCTCAGCCTGAATCTCTGAGCCCTTCTGGAAAA | |
| | GAACCTGCCGCCAGCTCTCCTAGCTCTAATAATACCGCCGCCACAACAGCC | |
| | GCCATTGTGCCTGGATCTCAGCTGATGCCTAGCAAGTCTCCTAGCACAGGC | |
| | ACAACAGAGATCAGCAGCCACGAATCTTCTCACGGAACACCTTCTCAGACC | |
| | ACCGCCAAGAATTGGGAGCTGACAGCCTCTGCCTCTCACCAGCCTCCAGGA | |
| | GTGTATCCTCAGGGCCACTCTGATACAACAGTGGCCATCAGCACATCTACA | |
| | GTGCTGCTGTGTGGACTGTCTGCCGTGTCTCTGCTGGCCTGTTACCTGAAG | |
| | TCTAGACAGACACCTCCTCTGGCCTCTGTGGAGATGGAGGCCATGGAAGCC | |
| | CTGCCTGTGACATGGGGAACAAGCAGCAGAGATGAAGACCTGGAGAATTGT | |
| | TCTCACCACCTGCGGGCGAAACGCTCTGGAAGCGGAGAGGGCAGAGGAAGT | |
| | CTTCTAACATGCGGTGACGTGGAGGAGAATCCCGGCCCT | |
| AP15T nucleotide | NNNATCCAGAATCCCGAGCCTGCGGTGTACCAGCTGAAGGACCCCCGCTCT | 238 |
| sequence | CAGGATAGCACACTGTGCCTGTTCACCGACTTTGATAGCCAGATCAACGTG | |
| | CCTAAAACAATGGAGTCCGGCACCTTCATCACCGACAAGNNNGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGNNNGTCNNNNNNCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCAATGGATTGGACCTGGATT | |
| | CTGTTTCTGGTGGCCGCTGCCACAAGAGTGCACAGCAACTGGGTGAATGTG | |
| | ATCAGCGACCTGAAGAAGATCGAGGATCTGATCCAGAGCATGCACATTGAT | |
| | GCCACCCTGTACACAGAATCTGATGTGCACCCTAGCTGTAAAGTGACCGCC | |
| | ATGAAGTGTTTTCTGCTGGAGCTGCAGGTGATTTCTCTGGAAAGCGGAGAT | |
| | GCCTCTATCCACGACACAGTGGAGAATCTGATCATCCTGGCCAACAATAGC | |
| | CTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTG | |
| | CAGATGTTCATCAATACAAGCTCTGGCGGAGGATCTGGAGGAGGCGGATCT | |
| | GGAGGAGGAGGCAGTGGAGGCGGAGGATCTGGCGGAGGATCTCTGCAGATT | |
| | ACATGCCCTCCTCCAATGTCTGTGGAGCACGCCGATATTTGGGTGAAGTCC | |
| | TACAGCCTGTACAGCAGAGAGAGATACATCTGCAACAGCGGCTTTAAGAGA | |
| | AAGGCCGGCACCTCTTCTCTGACAGAGTGCGTGCTGAATAAGGCCACAAAT | |
| | GTGGCCCACTGGACAACACCTAGCCTGAAGTGCATTAGAGATCCTGCCCTG | |
| | GTCCACCAGAGGCCTGCCCCTCCATCTACAGTGACAACAGCCGGAGTGACA | |
| | CCTCAGCCTGAATCTCTGAGCCCTTCTGGAAAAGAACCTGCCGCCAGCTCT | |
| | CCTAGCTCTAATAATACCGCCGCCACAACAGCCGCCATTGTGCCTGGATCT | |
| | CAGCTGATGCCTAGCAAGTCTCCTAGCACAGGCACAACAGAGATCAGCAGC | |
| | CACGAATCTTCTCACGGAACACCTTCTCAGACCACCGCCAAGAATTGGGAG | |
| | CTGACAGCCTCTGCCTCTCACCAGCCTCCAGGAGTGTATCCTCAGGGCCAC | |
| | TCTGATACAACAGTGGCCATCAGCACATCTACAGTGCTGCTGTGTGGACTG | |
| | TCTGCCGTGTCTCTGCTGGCCTGTTACCTGAAGTCTAGACAGACACCTCCT | |
| | CTGGCCTCTGTGGAGATGGAGGCCATGGAAGCCCTGCCTGTGACATGGGGA | |
| | ACAAGCAGCAGAGATGAAGACCTGGAGAATTGTTCTCACCACCTGCGGGCG | |
| | AAACGCTCTGGAAGCGGAGAGGGCAGAGGAAGTCTTCTAACATGCGGTGAC | |
| | GTGGAGGAGAATCCCGGCCCT | |
| | NNN at positions 1-3 make up a codon that encodes Asn, Asp, His, or Tyr; | |
| | NNN at positions 142-144 make up a codon that encodes Thr or Cys; | |
| | NNN at positions 334-336 make up a codon that encodes Ser, Ala, Val, Leu, | |
| | Ile, Pro, Phe, Met, or Trp; | |
| | NNN at positions 340-342 make up a codon that encodes Met, Ala, Val, Leu, | |
| | Ile, Pro, Phe, or Trp; | |
| | NNN at positions 343-345 make up a codon that encodes Gly, Ala, Val, Leu, | |
| | Ile, Pro, Phe, Met, or Trp | |
| AT15 nucleotide | NNNATCCAGAATCCCGAGCCTGCGGTGTACCAGCTGAAGGACCCCCGCTCT | 239 |
| sequence | CAGGATAGCACACTGTGCCTGTTCACCGACTTTGATAGCCAGATCAACGTG | |
| | CCTAAAACAATGGAGTCCGGCACCTTCATCACCGACAAGNNNGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGNNNGTCNNNNNNCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCTATGGATTGGACCTGGATTCTG | |
| | TTTCTGGTGGCCGCTGCCACAAGAGTGCACAGCAACTGGGTGAATGTGATC | |
| | AGCGACCTGAAGAAGATCGAGGATCTGATCCAGAGCATGCACATTGATGCC | |
| | ACCCTGTACACAGAATCTGATGTGCACCCTAGCTGTAAAGTGACCGCCATG | |
| | AAGTGTTTTCTGCTGGAGCTGCAGGTGATTTCTCTGGAAAGCGGAGATGCC | |
| | TCTATCCACGACACAGTGGAGAATCTGATCATCCTGGCCAACAATAGCCTG | |
| | AGCAGCAATGGCAATGTGACAGAGTCTGGCTGTAAGGAGTGTGAGGAGCTG | |
| | GAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGA | |
| | GGAGGAGGCAGTGGAGGCGGAGGATCTGGCGGAGGATCTCTGCAGATTACA | |
| | TGCCCTCCTCCAATGTCTGTGGAGCACGCCGATATTTGGGTGAAGTCCTAC | |
| | AGCCTGTACAGCAGAGAGAGATACATCTGCAACAGCGGCTTTAAGAGAAAG | |
| | GCCGGCACCTCTTCTCTGACAGAGTGCGTGCTGAATAAGGCCACAAATGTG | |
| | GCCCACTGGACAACACCTAGCCTGAAGTGCATTAGAGATCCTGCCCTGGTC | |
| | CACCAGAGGCCTGCCCCTCCATCTACAGTGACAACAGCCGGAGTGACACCT | |
| | CAGCCTGAATCTCTGAGCCCTTCTGGAAAAGAACCTGCCGCCAGCTCTCCT | |
| | AGCTCTAATAATACCGCCGCCACAACAGCCGCCATTGTGCCTGGATCTCAG | |
| | CTGATGCCTAGCAAGTCTCCTAGCACAGGCACAACAGAGATCAGCAGCCAC | |
| | GAATCTTCTCACGGAACACCTTCTCAGACCACCGCCAAGAATTGGGAGCTG | |
| | ACAGCCTCTGCCTCTCACCAGCCTCCAGGAGTGTATCCTCAGGGCCACTCT | |
| | GATACAACAGTGGCCATCAGCACATCTACAGTGCTGCTGTGTGGACTGTCT | |
| | GCCGTGTCTCTGCTGGCCTGTTACCTGAAGTCTAGACAGACACCTCCTCTG | |
| | GCCTCTGTGGAGATGGAGGCCATGGAAGCCCTGCCTGTGACATGGGGAACA | |
| | AGCAGCAGAGATGAAGACCTGGAGAATTGTTCTCACCACCTG | |
| | NNN at positions 1-3 make up a codon that encodes Asn, Asp, His, or Tyr; | |
| | NNN at positions 142-144 make up a codon that encodes Thr or Cys; | |
| | NNN at positions 334-336 make up a codon that encodes Ser, Ala, Val, Leu, | |
| | Ile, Pro, Phe, Met, or Trp; | |
| | NNN at positions 340-342 make up a codon that encodes Met, Ala, Val, Leu, | |
| | Ile, Pro, Phe, or Trp; | |
| | NNN at positions 343-345 make up a codon that encodes Gly, Ala, Val, Leu, | |
| | Ile, Pro, Phe, Met, or Trp | |
| 15P nucleotide | ATGGATTGGACCTGGATTCTGTTTCTGGTGGCCGCTGCCACAAGAGTGCAC | 240 |
| sequence | AGCAACTGGGTGAATGTGATCAGCGACCTGAAGAAGATCGAGGATCTGATC | |
| | CAGAGCATGCACATTGATGCCACCCTGTACACAGAATCTGATGTGCACCCT | |
| | AGCTGTAAAGTGACCGCCATGAAGTGTTTTCTGCTGGAGCTGCAGGTGATT | |
| | TCTCTGGAAAGCGGAGATGCCTCTATCCACGACACAGTGGAGAATCTGATC | |
| | ATCCTGGCCAACAATAGCCTGAGCAGCAATGGCAATGTGACAGAGTCTGGC | |
| | TGTAAGGAGTGTGAGGAGCTGGAGGAGAAGAACATCAAGGAGTTTCTGCAG | |
| | AGCTTTGTGCACATCGTGCAGATGTTCATCAATACAAGCTCTGGCGGAGGA | |
| | TCTGGAGGAGGCGGATCTGGAGGAGGAGGCAGTGGAGGCGGAGGATCTGGC | |
| | GGAGGATCTCTGCAGATTACATGCCCTCCTCCAATGTCTGTGGAGCACGCC | |
| | GATATTTGGGTGAAGTCCTACAGCCTGTACAGCAGAGAGAGATACATCTGC | |
| | AACAGCGGCTTTAAGAGAAAGGCCGGCACCTCTTCTCTGACAGAGTGCGTG | |
| | CTGAATAAGGCCACAAATGTGGCCCACTGGACAACACCTAGCCTGAAGTGC | |
| | ATTAGAGATCCTGCCCTGGTCCACCAGAGGCCTGCCCCTCCATCTACAGTG | |
| | ACAACAGCCGGAGTGACACCTCAGCCTGAATCTCTGAGCCCTTCTGGAAAA | |
| | GAACCTGCCGCCAGCTCTCCTAGCTCTAATAATACCGCCGCCACAACAGCC | |
| | GCCATTGTGCCTGGATCTCAGCTGATGCCTAGCAAGTCTCCTAGCACAGGC | |
| | ACAACAGAGATCAGCAGCCACGAATCTTCTCACGGAACACCTTCTCAGACC | |
| | ACCGCCAAGAATTGGGAGCTGACAGCCTCTGCCTCTCACCAGCCTCCAGGA | |
| | GTGTATCCTCAGGGCCACTCTGATACAACAGTGGCCATCAGCACATCTACA | |
| | GTGCTGCTGTGTGGACTGTCTGCCGTGTCTCTGCTGGCCTGTTACCTGAAG | |
| | TCTAGACAGACACCTCCTCTGGCCTCTGTGGAGATGGAGGCCATGGAAGCC | |
| | CTGCCTGTGACATGGGGAACAAGCAGCAGAGATGAAGACCTGGAGAATTGT | |
| | TCTCACCACCTGCGGGCGAAACGCTCTGGAAGCGGAGCGACCAATTTCAGC | |
| | CTGCTGAAGCAGGCGGGCGATGTGGAGGAGAACCCTGGCCCA | |
| AT15P nucleotide | NNNATCCAGAATCCCGAGCCTGCGGTGTACCAGCTGAAGGACCCCCGCTCT | 241 |
| sequence | CAGGATAGCACACTGTGCCTGTTCACCGACTTTGATAGCCAGATCAACGTG | |
| | CCTAAAACAATGGAGTCCGGCACCTTCATCACCGACAAGNNNGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGNNNGTCNNNNNNCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCTATGGATTGGACCTGGATTCTG | |
| | TTTCTGGTGGCCGCTGCCACAAGAGTGCACAGCAACTGGGTGAATGTGATC | |
| | AGCGACCTGAAGAAGATCGAGGATCTGATCCAGAGCATGCACATTGATGCC | |
| | ACCCTGTACACAGAATCTGATGTGCACCCTAGCTGTAAAGTGACCGCCATG | |
| | AAGTGTTTTCTGCTGGAGCTGCAGGTGATTTCTCTGGAAAGCGGAGATGCC | |
| | TCTATCCACGACACAGTGGAGAATCTGATCATCCTGGCCAACAATAGCCTG | |
| | AGCAGCAATGGCAATGTGACAGAGTCTGGCTGTAAGGAGTGTGAGGAGCTG | |
| | GAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGA | |
| | GGAGGAGGCAGTGGAGGCGGAGGATCTGGCGGAGGATCTCTGCAGATTACA | |
| | TGCCCTCCTCCAATGTCTGTGGAGCACGCCGATATTTGGGTGAAGTCCTAC | |
| | AGCCTGTACAGCAGAGAGAGATACATCTGCAACAGCGGCTTTAAGAGAAAG | |
| | GCCGGCACCTCTTCTCTGACAGAGTGCGTGCTGAATAAGGCCACAAATGTG | |
| | GCCCACTGGACAACACCTAGCCTGAAGTGCATTAGAGATCCTGCCCTGGTC | |
| | CACCAGAGGCCTGCCCCTCCATCTACAGTGACAACAGCCGGAGTGACACCT | |
| | CAGCCTGAATCTCTGAGCCCTTCTGGAAAAGAACCTGCCGCCAGCTCTCCT | |
| | AGCTCTAATAATACCGCCGCCACAACAGCCGCCATTGTGCCTGGATCTCAG | |
| | CTGATGCCTAGCAAGTCTCCTAGCACAGGCACAACAGAGATCAGCAGCCAC | |
| | GAATCTTCTCACGGAACACCTTCTCAGACCACCGCCAAGAATTGGGAGCTG | |
| | ACAGCCTCTGCCTCTCACCAGCCTCCAGGAGTGTATCCTCAGGGCCACTCT | |
| | GATACAACAGTGGCCATCAGCACATCTACAGTGCTGCTGTGTGGACTGTCT | |
| | GCCGTGTCTCTGCTGGCCTGTTACCTGAAGTCTAGACAGACACCTCCTCTG | |
| | GCCTCTGTGGAGATGGAGGCCATGGAAGCCCTGCCTGTGACATGGGGAACA | |
| | AGCAGCAGAGATGAAGACCTGGAGAATTGTTCTCACCACCTGCGGGCGAAA | |
| | CGCTCTGGAAGCGGAGCGACCAATTTCAGCCTGCTGAAGCAGGCGGGCGAT | |
| | GTGGAGGAGAACCCTGGCCCA | |
| | NNN at positions 1-3 make up a codon that encodes Asn, Asp, His, or Tyr; | |
| | NNN at positions 142-144 make up a codon that encodes Thr or Cys; | |
| | NNN at positions 334-336 make up a codon that encodes Ser, Ala, Val, Leu, | |
| | Ile, Pro, Phe, Met, or Trp; | |
| | NNN at positions 340-342 make up a codon that encodes Met, Ala, Val, Leu, | |
| | Ile, Pro, Phe, or Trp; | |
| | NNN at positions 343-345 make up a codon that encodes Gly, Ala, Val, Leu, | |
| | Ile, Pro, Phe, Met, or Trp | |
| BP15T nucleotide | GAGGACCTGAGGAACGTGACCCCACCTAAAGTGAGCCTGTTCGAGCCATCC | 242 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGNNNACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGCGACCAATTTCAGCCTG | |
| | CTGAAGCAGGCGGGCGATGTGGAGGAGAACCCTGGCCCAATGGATTGGACC | |
| | TGGATTCTGTTTCTGGTGGCCGCTGCCACAAGAGTGCACAGCAACTGGGTG | |
| | AATGTGATCAGCGACCTGAAGAAGATCGAGGATCTGATCCAGAGCATGCAC | |
| | ATTGATGCCACCCTGTACACAGAATCTGATGTGCACCCTAGCTGTAAAGTG | |
| | ACCGCCATGAAGTGTTTTCTGCTGGAGCTGCAGGTGATTTCTCTGGAAAGC | |
| | GGAGATGCCTCTATCCACGACACAGTGGAGAATCTGATCATCCTGGCCAAC | |
| | AATAGCCTGAGCAGCAATGGCAATGTGACAGAGTCTGGCTGTAAGGAGTGT | |
| | GAGGAGCTGGAGGAGAAGAACATCAAGGAGTTTCTGCAGAGCTTTGTGCAC | |
| | ATCGTGCAGATGTTCATCAATACAAGCTCTGGCGGAGGATCTGGAGGAGGC | |
| | GGATCTGGAGGAGGAGGCAGTGGAGGCGGAGGATCTGGCGGAGGATCTCTG | |
| | CAGATTACATGCCCTCCTCCAATGTCTGTGGAGCACGCCGATATTTGGGTG | |
| | AAGTCCTACAGCCTGTACAGCAGAGAGAGATACATCTGCAACAGCGGCTTT | |
| | AAGAGAAAGGCCGGCACCTCTTCTCTGACAGAGTGCGTGCTGAATAAGGCC | |
| | ACAAATGTGGCCCACTGGACAACACCTAGCCTGAAGTGCATTAGAGATCCT | |
| | GCCCTGGTCCACCAGAGGCCTGCCCCTCCATCTACAGTGACAACAGCCGGA | |
| | GTGACACCTCAGCCTGAATCTCTGAGCCCTTCTGGAAAAGAACCTGCCGCC | |
| | AGCTCTCCTAGCTCTAATAATACCGCCGCCACAACAGCCGCCATTGTGCCT | |
| | GGATCTCAGCTGATGCCTAGCAAGTCTCCTAGCACAGGCACAACAGAGATC | |
| | AGCAGCCACGAATCTTCTCACGGAACACCTTCTCAGACCACCGCCAAGAAT | |
| | TGGGAGCTGACAGCCTCTGCCTCTCACCAGCCTCCAGGAGTGTATCCTCAG | |
| | GGCCACTCTGATACAACAGTGGCCATCAGCACATCTACAGTGCTGCTGTGT | |
| | GGACTGTCTGCCGTGTCTCTGCTGGCCTGTTACCTGAAGTCTAGACAGACA | |
| | CCTCCTCTGGCCTCTGTGGAGATGGAGGCCATGGAAGCCCTGCCTGTGACA | |
| | TGGGGAACAAGCAGCAGAGATGAAGACCTGGAGAATTGTTCTCACCACCTG | |
| | CGGGCGAAACGCTCTGGAAGCGGAGAGGGCAGAGGAAGTCTTCTAACATGC | |
| | GGTGACGTGGAGGAGAATCCCGGCCCT | |
| | NNN at positions 169-171 make up a codon that encodes Ser or Cys | |
| BT15P nucleotide | GAGGACCTGAGGAACGTGACCCCACCTAAAGTGAGCCTGTTCGAGCCATCC | 243 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGNNNACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGAGGGCAGAGGAAGTCTT | |
| | CTAACATGCGGTGACGTGGAGGAGAATCCCGGCCCTATGGATTGGACCTGG | |
| | ATTCTGTTTCTGGTGGCCGCTGCCACAAGAGTGCACAGCAACTGGGTGAAT | |
| | GTGATCAGCGACCTGAAGAAGATCGAGGATCTGATCCAGAGCATGCACATT | |
| | GATGCCACCCTGTACACAGAATCTGATGTGCACCCTAGCTGTAAAGTGACC | |
| | GCCATGAAGTGTTTTCTGCTGGAGCTGCAGGTGATTTCTCTGGAAAGCGGA | |
| | GATGCCTCTATCCACGACACAGTGGAGAATCTGATCATCCTGGCCAACAAT | |
| | AGCCTGAGCAGCAATGGCAATGTGACAGAGTCTGGCTGTAAGGAGTGTGAG | |
| | GAGCTGGAGGAGAAGAACATCAAGGAGTTTCTGCAGAGCTTTGTGCACATC | |
| | GTGCAGATGTTCATCAATACAAGCTCTGGCGGAGGATCTGGAGGAGGCGGA | |
| | TCTGGAGGAGGAGGCAGTGGAGGCGGAGGATCTGGCGGAGGATCTCTGCAG | |
| | ATTACATGCCCTCCTCCAATGTCTGTGGAGCACGCCGATATTTGGGTGAAG | |
| | TCCTACAGCCTGTACAGCAGAGAGAGATACATCTGCAACAGCGGCTTTAAG | |
| | AGAAAGGCCGGCACCTCTTCTCTGACAGAGTGCGTGCTGAATAAGGCCACA | |
| | AATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTG | |
| | ACACCTCAGCCTGAATCTCTGAGCCCTTCTGGAAAAGAACCTGCCGCCAGC | |
| | TCTCCTAGCTCTAATAATACCGCCGCCACAACAGCCGCCATTGTGCCTGGA | |
| | TCTCAGCTGATGCCTAGCAAGTCTCCTAGCACAGGCACAACAGAGATCAGC | |
| | AGCCACGAATCTTCTCACGGAACACCTTCTCAGACCACCGCCAAGAATTGG | |
| | GAGCTGACAGCCTCTGCCTCTCACCAGCCTCCAGGAGTGTATCCTCAGGGC | |
| | CACTCTGATACAACAGTGGCCATCAGCACATCTACAGTGCTGCTGTGTGGA | |
| | CTGTCTGCCGTGTCTCTGCTGGCCTGTTACCTGAAGTCTAGACAGACACCT | |
| | CCTCTGGCCTCTGTGGAGATGGAGGCCATGGAAGCCCTGCCTGTGACATGG | |
| | GGAACAAGCAGCAGAGATGAAGACCTGGAGAATTGTTCTCACCACCTGCGG | |
| | GCGAAACGCTCTGGAAGCGGAGCGACCAATTTCAGCCTGCTGAAGCAGGCG | |
| | GGCGATGTGGAGGAGAACCCTGGCCCA | |
| | NNN at positions 169-171 make up a codon that encodes Ser or Cys | |

TABLE 11B
Exemplary polynucleotide sequences for use in polycistronic expression
cassette.

| | | SEQ ID |
|---|---|---|
| Description | Sequence | NO: |
| AP nucleotide | AACATCCAGAATCCCGAGCCTGCGGTGTACCAGCTGAAGGACCCCCGCTCT | 250 |
| sequence | CAGGATAGCACACTGTGCCTGTTCACCGACTTTGATAGCCAGATCAACGTG | |
| | CCTAAAACAATGGAGTCCGGCACCTTCATCACCGACAAGTGCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCA | |
| BT nucleotide | GAGGACCTGAGGAACGTGACCCCACCTAAAGTGAGCCTGTTCGAGCCATCC | 251 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGTGCACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGAGGGCAGAGGAAGTCTT | |
| | CTAACATGCGGTGACGTGGAGGAGAATCCCGGCCCT | |
| BT15 nucleotide | GAGGACCTGAGGAACGTGACCCCACCTAAAGTGAGCCTGTTCGAGCCATCC | 252 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGTGCACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGAGGGCAGAGGAAGTCTT | |
| | CTAACATGCGGTGACGTGGAGGAGAATCCCGGCCCTATGGATTGGACCTGG | |
| | ATTCTGTTTCTGGTGGCCGCTGCCACAAGAGTGCACAGCAACTGGGTGAAT | |
| | GTGATCAGCGACCTGAAGAAGATCGAGGATCTGATCCAGAGCATGCACATT | |
| | GATGCCACCCTGTACACAGAATCTGATGTGCACCCTAGCTGTAAAGTGACC | |
| | GCCATGAAGTGTTTTCTGCTGGAGCTGCAGGTGATTTCTCTGGAAAGCGGA | |
| | GATGCCTCTATCCACGACACAGTGGAGAATCTGATCATCCTGGCCAACAAT | |
| | AGCCTGAGCAGCAATGGCAATGTGACAGAGTCTGGCTGTAAGGAGTGTGAG | |
| | GAGCTGGAGGAGAAGAACATCAAGGAGTTTCTGCAGAGCTTTGTGCACATC | |
| | GTGCAGATGTTCATCAATACAAGCTCTGGCGGAGGATCTGGAGGAGGCGGA | |
| | TCTGGAGGAGGAGGCAGTGGAGGCGGAGGATCTGGCGGAGGATCTCTGCAG | |
| | ATTACATGCCCTCCTCCAATGTCTGTGGAGCACGCCGATATTTGGGTGAAG | |
| | TCCTACAGCCTGTACAGCAGAGAGAGATACATCTGCAACAGCGGCTTTAAG | |
| | AGAAAGGCCGGCACCTCTTCTCTGACAGAGTGCGTGCTGAATAAGGCCACA | |
| | AATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTG | |
| | ACACCTCAGCCTGAATCTCTGAGCCCTTCTGGAAAAGAACCTGCCGCCAGC | |
| | TCTCCTAGCTCTAATAATACCGCCGCCACAACAGCCGCCATTGTGCCTGGA | |
| | TCTCAGCTGATGCCTAGCAAGTCTCCTAGCACAGGCACAACAGAGATCAGC | |
| | AGCCACGAATCTTCTCACGGAACACCTTCTCAGACCACCGCCAAGAATTGG | |
| | GAGCTGACAGCCTCTGCCTCTCACCAGCCTCCAGGAGTGTATCCTCAGGGC | |
| | CACTCTGATACAACAGTGGCCATCAGCACATCTACAGTGCTGCTGTGTGGA | |
| | CTGTCTGCCGTGTCTCTGCTGGCCTGTTACCTGAAGTCTAGACAGACACCT | |
| | CCTCTGGCCTCTGTGGAGATGGAGGCCATGGAAGCCCTGCCTGTGACATGG | |
| | GGAACAAGCAGCAGAGATGAAGACCTGGAGAATTGTTCTCACCACCTG | |
| AT nucleotide | AACATCCAGAATCCCGAGCCTGCGGTGTACCAGCTGAAGGACCCCCGCTCT | 253 |
| sequence | CAGGATAGCACACTGTGCCTGTTCACCGACTTTGATAGCCAGATCAACGTG | |
| | CCTAAAACAATGGAGTCCGGCACCTTCATCACCGACAAGTGCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCT | |
| BP nucleotide | GAGGACCTGAGGAACGTGACCCCACCTAAAGTGAGCCTGTTCGAGCCATCC | 254 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGTGCACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGCGACCAATTTCAGCCTG | |
| | CTGAAGCAGGCGGGCGATGTGGAGGAGAACCCTGGCCCA | |
| BP15 nucleotide | GAGGACCTGAGGAACGTGACCCCACCTAAAGTGAGCCTGTTCGAGCCATCC | 255 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGTGCACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGCGACCAATTTCAGCCTG | |
| | CTGAAGCAGGCGGGCGATGTGGAGGAGAACCCTGGCCCAATGGATTGGACC | |
| | TGGATTCTGTTTCTGGTGGCCGCTGCCACAAGAGTGCACAGCAACTGGGTG | |
| | AATGTGATCAGCGACCTGAAGAAGATCGAGGATCTGATCCAGAGCATGCAC | |
| | ATTGATGCCACCCTGTACACAGAATCTGATGTGCACCCTAGCTGTAAAGTG | |
| | ACCGCCATGAAGTGTTTTCTGCTGGAGCTGCAGGTGATTTCTCTGGAAAGC | |
| | GGAGATGCCTCTATCCACGACACAGTGGAGAATCTGATCATCCTGGCCAAC | |
| | AATAGCCTGAGCAGCAATGGCAATGTGACAGAGTCTGGCTGTAAGGAGTGT | |
| | GAGGAGCTGGAGGAGAAGAACATCAAGGAGTTTCTGCAGAGCTTTGTGCAC | |
| | ATCGTGCAGATGTTCATCAATACAAGCTCTGGCGGAGGATCTGGAGGAGGC | |
| | GGATCTGGAGGAGGAGGCAGTGGAGGCGGAGGATCTGGCGGAGGATCTCTG | |
| | CAGATTACATGCCCTCCTCCAATGTCTGTGGAGCACGCCGATATTTGGGTG | |
| | AAGTCCTACAGCCTGTACAGCAGAGAGAGATACATCTGCAACAGCGGCTTT | |
| | AAGAGAAAGGCCGGCACCTCTTCTCTGACAGAGTGCGTGCTGAATAAGGCC | |
| | ACAAATGTGGCCCACTGGACAACACCTAGCCTGAAGTGCATTAGAGATCCT | |
| | GCCCTGGTCCACCAGAGGCCTGCCCCTCCATCTACAGTGACAACAGCCGGA | |
| | GTGACACCTCAGCCTGAATCTCTGAGCCCTTCTGGAAAAGAACCTGCCGCC | |
| | AGCTCTCCTAGCTCTAATAATACCGCCGCCACAACAGCCGCCATTGTGCCT | |
| | GGATCTCAGCTGATGCCTAGCAAGTCTCCTAGCACAGGCACAACAGAGATC | |
| | AGCAGCCACGAATCTTCTCACGGAACACCTTCTCAGACCACCGCCAAGAAT | |
| | TGGGAGCTGACAGCCTCTGCCTCTCACCAGCCTCCAGGAGTGTATCCTCAG | |
| | GGCCACTCTGATACAACAGTGGCCATCAGCACATCTACAGTGCTGCTGTGT | |
| | GGACTGTCTGCCGTGTCTCTGCTGGCCTGTTACCTGAAGTCTAGACAGACA | |
| | CCTCCTCTGGCCTCTGTGGAGATGGAGGCCATGGAAGCCCTGCCTGTGACA | |
| | TGGGGAACAAGCAGCAGAGATGAAGACCTGGAGAATTGTTCTCACCACCTG | |
| AP15 nucleotide | AACATCCAGAATCCCGAGCCTGCGGTGTACCAGCTGAAGGACCCCCGCTCT | |
| sequence | CAGGATAGCACACTGTGCCTGTTCACCGACTTTGATAGCCAGATCAACGTG | 256 |
| | CCTAAAACAATGGAGTCCGGCACCTTCATCACCGACAAGTGCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCAATGGATTGGACCTGGATT | |
| | CTGTTTCTGGTGGCCGCTGCCACAAGAGTGCACAGCAACTGGGTGAATGTG | |
| | ATCAGCGACCTGAAGAAGATCGAGGATCTGATCCAGAGCATGCACATTGAT | |
| | GCCACCCTGTACACAGAATCTGATGTGCACCCTAGCTGTAAAGTGACCGCC | |
| | ATGAAGTGTTTTCTGCTGGAGCTGCAGGTGATTTCTCTGGAAAGCGGAGAT | |
| | GCCTCTATCCACGACACAGTGGAGAATCTGATCATCCTGGCCAACAATAGC | |
| | CTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTG | |
| | CAGATGTTCATCAATACAAGCTCTGGCGGAGGATCTGGAGGAGGCGGATCT | |
| | GGAGGAGGAGGCAGTGGAGGCGGAGGATCTGGCGGAGGATCTCTGCAGATT | |
| | ACATGCCCTCCTCCAATGTCTGTGGAGCACGCCGATATTTGGGTGAAGTCC | |
| | TACAGCCTGTACAGCAGAGAGAGATACATCTGCAACAGCGGCTTTAAGAGA | |
| | AAGGCCGGCACCTCTTCTCTGACAGAGTGCGTGCTGAATAAGGCCACAAAT | |
| | GTGGCCCACTGGACAACACCTAGCCTGAAGTGCATTAGAGATCCTGCCCTG | |
| | GTCCACCAGAGGCCTGCCCCTCCATCTACAGTGACAACAGCCGGAGTGACA | |
| | CCTCAGCCTGAATCTCTGAGCCCTTCTGGAAAAGAACCTGCCGCCAGCTCT | |
| | CCTAGCTCTAATAATACCGCCGCCACAACAGCCGCCATTGTGCCTGGATCT | |
| | CAGCTGATGCCTAGCAAGTCTCCTAGCACAGGCACAACAGAGATCAGCAGC | |
| | CACGAATCTTCTCACGGAACACCTTCTCAGACCACCGCCAAGAATTGGGAG | |
| | CTGACAGCCTCTGCCTCTCACCAGCCTCCAGGAGTGTATCCTCAGGGCCAC | |
| | TCTGATACAACAGTGGCCATCAGCACATCTACAGTGCTGCTGTGTGGACTG | |
| | TCTGCCGTGTCTCTGCTGGCCTGTTACCTGAAGTCTAGACAGACACCTCCT | |
| | CTGGCCTCTGTGGAGATGGAGGCCATGGAAGCCCTGCCTGTGACATGGGGA | |
| | ACAAGCAGCAGAGATGAAGACCTGGAGAATTGTTCTCACCACCTG | |
| AP15T nucleotide | AACATCCAGAATCCCGAGCCTGCGGTGTACCAGCTGAAGGACCCCCGCTCT | 258 |
| sequence | CAGGATAGCACACTGTGCCTGTTCACCGACTTTGATAGCCAGATCAACGTG | |
| | CCTAAAACAATGGAGTCCGGCACCTTCATCACCGACAAGTGCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCAATGGATTGGACCTGGATT | |
| | CTGTTTCTGGTGGCCGCTGCCACAAGAGTGCACAGCAACTGGGTGAATGTG | |
| | ATCAGCGACCTGAAGAAGATCGAGGATCTGATCCAGAGCATGCACATTGAT | |
| | GCCACCCTGTACACAGAATCTGATGTGCACCCTAGCTGTAAAGTGACCGCC | |
| | ATGAAGTGTTTTCTGCTGGAGCTGCAGGTGATTTCTCTGGAAAGCGGAGAT | |
| | GCCTCTATCCACGACACAGTGGAGAATCTGATCATCCTGGCCAACAATAGC | |
| | CTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTG | |
| | CAGATGTTCATCAATACAAGCTCTGGCGGAGGATCTGGAGGAGGCGGATCT | |
| | GGAGGAGGAGGCAGTGGAGGCGGAGGATCTGGCGGAGGATCTCTGCAGATT | |
| | ACATGCCCTCCTCCAATGTCTGTGGAGCACGCCGATATTTGGGTGAAGTCC | |
| | TACAGCCTGTACAGCAGAGAGAGATACATCTGCAACAGCGGCTTTAAGAGA | |
| | AAGGCCGGCACCTCTTCTCTGACAGAGTGCGTGCTGAATAAGGCCACAAAT | |
| | GTGGCCCACTGGACAACACCTAGCCTGAAGTGCATTAGAGATCCTGCCCTG | |
| | GTCCACCAGAGGCCTGCCCCTCCATCTACAGTGACAACAGCCGGAGTGACA | |
| | CCTCAGCCTGAATCTCTGAGCCCTTCTGGAAAAGAACCTGCCGCCAGCTCT | |
| | CCTAGCTCTAATAATACCGCCGCCACAACAGCCGCCATTGTGCCTGGATCT | |
| | CAGCTGATGCCTAGCAAGTCTCCTAGCACAGGCACAACAGAGATCAGCAGC | |
| | CACGAATCTTCTCACGGAACACCTTCTCAGACCACCGCCAAGAATTGGGAG | |
| | CTGACAGCCTCTGCCTCTCACCAGCCTCCAGGAGTGTATCCTCAGGGCCAC | |
| | TCTGATACAACAGTGGCCATCAGCACATCTACAGTGCTGCTGTGTGGACTG | |
| | TCTGCCGTGTCTCTGCTGGCCTGTTACCTGAAGTCTAGACAGACACCTCCT | |
| | CTGGCCTCTGTGGAGATGGAGGCCATGGAAGCCCTGCCTGTGACATGGGGA | |
| | ACAAGCAGCAGAGATGAAGACCTGGAGAATTGTTCTCACCACCTGCGGGCG | |
| | AAACGCTCTGGAAGCGGAGAGGGCAGAGGAAGTCTTCTAACATGCGGTGAC | |
| | GTGGAGGAGAATCCCGGCCCT | |
| AT15 nucleotide | AACATCCAGAATCCCGAGCCTGCGGTGTACCAGCTGAAGGACCCCCGCTCT | 259 |
| sequence | CAGGATAGCACACTGTGCCTGTTCACCGACTTTGATAGCCAGATCAACGTG | |
| | CCTAAAACAATGGAGTCCGGCACCTTCATCACCGACAAGTGCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCTATGGATTGGACCTGGATTCTG | |
| | TTTCTGGTGGCCGCTGCCACAAGAGTGCACAGCAACTGGGTGAATGTGATC | |
| | AGCGACCTGAAGAAGATCGAGGATCTGATCCAGAGCATGCACATTGATGCC | |
| | ACCCTGTACACAGAATCTGATGTGCACCCTAGCTGTAAAGTGACCGCCATG | |
| | AAGTGTTTTCTGCTGGAGCTGCAGGTGATTTCTCTGGAAAGCGGAGATGCC | |
| | TCTATCCACGACACAGTGGAGAATCTGATCATCCTGGCCAACAATAGCCTG | |
| | AGCAGCAATGGCAATGTGACAGAGTCTGGCTGTAAGGAGTGTGAGGAGCTG | |
| | GAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGA | |
| | GGAGGAGGCAGTGGAGGCGGAGGATCTGGCGGAGGATCTCTGCAGATTACA | |
| | TGCCCTCCTCCAATGTCTGTGGAGCACGCCGATATTTGGGTGAAGTCCTAC | |
| | AGCCTGTACAGCAGAGAGAGATACATCTGCAACAGCGGCTTTAAGAGAAAG | |
| | GCCGGCACCTCTTCTCTGACAGAGTGCGTGCTGAATAAGGCCACAAATGTG | |
| | GCCCACTGGACAACACCTAGCCTGAAGTGCATTAGAGATCCTGCCCTGGTC | |
| | CACCAGAGGCCTGCCCCTCCATCTACAGTGACAACAGCCGGAGTGACACCT | |
| | CAGCCTGAATCTCTGAGCCCTTCTGGAAAAGAACCTGCCGCCAGCTCTCCT | |
| | AGCTCTAATAATACCGCCGCCACAACAGCCGCCATTGTGCCTGGATCTCAG | |
| | CTGATGCCTAGCAAGTCTCCTAGCACAGGCACAACAGAGATCAGCAGCCAC | |
| | GAATCTTCTCACGGAACACCTTCTCAGACCACCGCCAAGAATTGGGAGCTG | |
| | ACAGCCTCTGCCTCTCACCAGCCTCCAGGAGTGTATCCTCAGGGCCACTCT | |
| | GATACAACAGTGGCCATCAGCACATCTACAGTGCTGCTGTGTGGACTGTCT | |
| | GCCGTGTCTCTGCTGGCCTGTTACCTGAAGTCTAGACAGACACCTCCTCTG | |
| | GCCTCTGTGGAGATGGAGGCCATGGAAGCCCTGCCTGTGACATGGGGAACA | |
| | AGCAGCAGAGATGAAGACCTGGAGAATTGTTCTCACCACCTG | |
| AT15P nucleotide | AACATCCAGAATCCCGAGCCTGCGGTGTACCAGCTGAAGGACCCCCGCTCT | 261 |
| sequence | CAGGATAGCACACTGTGCCTGTTCACCGACTTTGATAGCCAGATCAACGTG | |
| | CCTAAAACAATGGAGTCCGGCACCTTCATCACCGACAAGTGCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCTATGGATTGGACCTGGATTCTG | |
| | TTTCTGGTGGCCGCTGCCACAAGAGTGCACAGCAACTGGGTGAATGTGATC | |
| | AGCGACCTGAAGAAGATCGAGGATCTGATCCAGAGCATGCACATTGATGCC | |
| | ACCCTGTACACAGAATCTGATGTGCACCCTAGCTGTAAAGTGACCGCCATG | |
| | AAGTGTTTTCTGCTGGAGCTGCAGGTGATTTCTCTGGAAAGCGGAGATGCC | |
| | TCTATCCACGACACAGTGGAGAATCTGATCATCCTGGCCAACAATAGCCTG | |
| | AGCAGCAATGGCAATGTGACAGAGTCTGGCTGTAAGGAGTGTGAGGAGCTG | |
| | GAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGA | |
| | GGAGGAGGCAGTGGAGGCGGAGGATCTGGCGGAGGATCTCTGCAGATTACA | |
| | TGCCCTCCTCCAATGTCTGTGGAGCACGCCGATATTTGGGTGAAGTCCTAC | |
| | AGCCTGTACAGCAGAGAGAGATACATCTGCAACAGCGGCTTTAAGAGAAAG | |
| | GCCGGCACCTCTTCTCTGACAGAGTGCGTGCTGAATAAGGCCACAAATGTG | |
| | GCCCACTGGACAACACCTAGCCTGAAGTGCATTAGAGATCCTGCCCTGGTC | |
| | CACCAGAGGCCTGCCCCTCCATCTACAGTGACAACAGCCGGAGTGACACCT | |
| | CAGCCTGAATCTCTGAGCCCTTCTGGAAAAGAACCTGCCGCCAGCTCTCCT | |
| | AGCTCTAATAATACCGCCGCCACAACAGCCGCCATTGTGCCTGGATCTCAG | |
| | CTGATGCCTAGCAAGTCTCCTAGCACAGGCACAACAGAGATCAGCAGCCAC | |
| | GAATCTTCTCACGGAACACCTTCTCAGACCACCGCCAAGAATTGGGAGCTG | |
| | ACAGCCTCTGCCTCTCACCAGCCTCCAGGAGTGTATCCTCAGGGCCACTCT | |
| | GATACAACAGTGGCCATCAGCACATCTACAGTGCTGCTGTGTGGACTGTCT | |
| | GCCGTGTCTCTGCTGGCCTGTTACCTGAAGTCTAGACAGACACCTCCTCTG | |
| | GCCTCTGTGGAGATGGAGGCCATGGAAGCCCTGCCTGTGACATGGGGAACA | |
| | AGCAGCAGAGATGAAGACCTGGAGAATTGTTCTCACCACCTGCGGGCGAAA | |
| | CGCTCTGGAAGCGGAGCGACCAATTTCAGCCTGCTGAAGCAGGCGGGCGAT | |
| | GTGGAGGAGAACCCTGGCCCA | |
| BP15T nucleotide | GAGGACCTGAGGAACGTGACCCCACCTAAAGTGAGCCTGTTCGAGCCATCC | 262 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGTGCACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGCGACCAATTTCAGCCTG | |
| | CTGAAGCAGGCGGGCGATGTGGAGGAGAACCCTGGCCCAATGGATTGGACC | |
| | TGGATTCTGTTTCTGGTGGCCGCTGCCACAAGAGTGCACAGCAACTGGGTG | |
| | AATGTGATCAGCGACCTGAAGAAGATCGAGGATCTGATCCAGAGCATGCAC | |
| | ATTGATGCCACCCTGTACACAGAATCTGATGTGCACCCTAGCTGTAAAGTG | |
| | ACCGCCATGAAGTGTTTTCTGCTGGAGCTGCAGGTGATTTCTCTGGAAAGC | |
| | GGAGATGCCTCTATCCACGACACAGTGGAGAATCTGATCATCCTGGCCAAC | |
| | AATAGCCTGAGCAGCAATGGCAATGTGACAGAGTCTGGCTGTAAGGAGTGT | |
| | GAGGAGCTGGAGGAGAAGAACATCAAGGAGTTTCTGCAGAGCTTTGTGCAC | |
| | ATCGTGCAGATGTTCATCAATACAAGCTCTGGCGGAGGATCTGGAGGAGGC | |
| | GGATCTGGAGGAGGAGGCAGTGGAGGCGGAGGATCTGGCGGAGGATCTCTG | |
| | CAGATTACATGCCCTCCTCCAATGTCTGTGGAGCACGCCGATATTTGGGTG | |
| | AAGTCCTACAGCCTGTACAGCAGAGAGAGATACATCTGCAACAGCGGCTTT | |
| | AAGAGAAAGGCCGGCACCTCTTCTCTGACAGAGTGCGTGCTGAATAAGGCC | |
| | ACAAATGTGGCCCACTGGACAACACCTAGCCTGAAGTGCATTAGAGATCCT | |
| | GCCCTGGTCCACCAGAGGCCTGCCCCTCCATCTACAGTGACAACAGCCGGA | |
| | GTGACACCTCAGCCTGAATCTCTGAGCCCTTCTGGAAAAGAACCTGCCGCC | |
| | AGCTCTCCTAGCTCTAATAATACCGCCGCCACAACAGCCGCCATTGTGCCT | |
| | GGATCTCAGCTGATGCCTAGCAAGTCTCCTAGCACAGGCACAACAGAGATC | |
| | AGCAGCCACGAATCTTCTCACGGAACACCTTCTCAGACCACCGCCAAGAAT | |
| | TGGGAGCTGACAGCCTCTGCCTCTCACCAGCCTCCAGGAGTGTATCCTCAG | |
| | GGCCACTCTGATACAACAGTGGCCATCAGCACATCTACAGTGCTGCTGTGT | |
| | GGACTGTCTGCCGTGTCTCTGCTGGCCTGTTACCTGAAGTCTAGACAGACA | |
| | CCTCCTCTGGCCTCTGTGGAGATGGAGGCCATGGAAGCCCTGCCTGTGACA | |
| | TGGGGAACAAGCAGCAGAGATGAAGACCTGGAGAATTGTTCTCACCACCTG | |
| | CGGGCGAAACGCTCTGGAAGCGGAGAGGGCAGAGGAAGTCTTCTAACATGC | |
| | GGTGACGTGGAGGAGAATCCCGGCCCT | |
| BT15P nucleotide | GAGGACCTGAGGAACGTGACCCCACCTAAAGTGAGCCTGTTCGAGCCATCC | 263 |
| sequence | AAGGCGGAGATCGCGAATAAGCAGAAAGCGACCCTGGTGTGCCTGGCGAGG | |
| | GGCTTCTTTCCCGATCACGTGGAGCTGTCCTGGTGGGTGAACGGCAAAGAG | |
| | GTGCACTCTGGCGTGTGCACAGACCCTCAGGCGTACAAGGAGAGCAATTAC | |
| | TCCTATTGTCTGTCTAGCAGACTGAGGGTGAGCGCGACCTTTTGGCACAAC | |
| | CCCCGGAATCACTTCCGCTGCCAGGTGCAGTTTCACGGCCTGTCCGAGGAG | |
| | GATAAATGGCCTGAGGGCTCTCCAAAGCCCGTGACACAGAATATCAGCGCG | |
| | GAGGCGTGGGGAAGAGCGGACTGTGGCATTACAAGCGCGTCCTATCAGCAG | |
| | GGCGTGCTGTCCGCGACCATCCTGTACGAGATTCTGCTGGGCAAGGCGACA | |
| | CTGTATGCGGTGCTGGTGTCCACCCTGGTGGTCATGGCGATGGTGAAGAGG | |
| | AAAAACTCTCGGGCGAAACGCTCTGGAAGCGGAGAGGGCAGAGGAAGTCTT | |
| | CTAACATGCGGTGACGTGGAGGAGAATCCCGGCCCTATGGATTGGACCTGG | |
| | ATTCTGTTTCTGGTGGCCGCTGCCACAAGAGTGCACAGCAACTGGGTGAAT | |
| | GTGATCAGCGACCTGAAGAAGATCGAGGATCTGATCCAGAGCATGCACATT | |
| | GATGCCACCCTGTACACAGAATCTGATGTGCACCCTAGCTGTAAAGTGACC | |
| | GCCATGAAGTGTTTTCTGCTGGAGCTGCAGGTGATTTCTCTGGAAAGCGGA | |
| | GATGCCTCTATCCACGACACAGTGGAGAATCTGATCATCCTGGCCAACAAT | |
| | AGCCTGAGCAGCAATGGCAATGTGACAGAGTCTGGCTGTAAGGAGTGTGAG | |
| | GAGCTGGAGGAGAAGAACATCAAGGAGTTTCTGCAGAGCTTTGTGCACATC | |
| | GTGCAGATGTTCATCAATACAAGCTCTGGCGGAGGATCTGGAGGAGGCGGA | |
| | TCTGGAGGAGGAGGCAGTGGAGGCGGAGGATCTGGCGGAGGATCTCTGCAG | |
| | ATTACATGCCCTCCTCCAATGTCTGTGGAGCACGCCGATATTTGGGTGAAG | |
| | TCCTACAGCCTGTACAGCAGAGAGAGATACATCTGCAACAGCGGCTTTAAG | |
| | AGAAAGGCCGGCACCTCTTCTCTGACAGAGTGCGTGCTGAATAAGGCCACA | |
| | AATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTG | |
| | ACACCTCAGCCTGAATCTCTGAGCCCTTCTGGAAAAGAACCTGCCGCCAGC | |
| | TCTCCTAGCTCTAATAATACCGCCGCCACAACAGCCGCCATTGTGCCTGGA | |
| | TCTCAGCTGATGCCTAGCAAGTCTCCTAGCACAGGCACAACAGAGATCAGC | |
| | AGCCACGAATCTTCTCACGGAACACCTTCTCAGACCACCGCCAAGAATTGG | |
| | GAGCTGACAGCCTCTGCCTCTCACCAGCCTCCAGGAGTGTATCCTCAGGGC | |
| | CACTCTGATACAACAGTGGCCATCAGCACATCTACAGTGCTGCTGTGTGGA | |
| | CTGTCTGCCGTGTCTCTGCTGGCCTGTTACCTGAAGTCTAGACAGACACCT | |
| | CCTCTGGCCTCTGTGGAGATGGAGGCCATGGAAGCCCTGCCTGTGACATGG | |
| | GGAACAAGCAGCAGAGATGAAGACCTGGAGAATTGTTCTCACCACCTGCGG | |
| | GCGAAACGCTCTGGAAGCGGAGCGACCAATTTCAGCCTGCTGAAGCAGGCG | |
| | GGCGATGTGGAGGAGAACCCTGGCCCA | |

TABLE 11C
Exemplary polynucleotide sequences for use in polycistronic expression
cassette.

| | | SEQ ID |
|---|---|---|
| Description | Sequence | NO: |
| AP nucleotide | AACATCCAGAATCCCGAGCCTGCGGTGTACCAGCTGAAGGACCCCCGCTCT | 270 |
| sequence | CAGGATAGCACACTGTGCCTGTTCACCGACTTTGATAGCCAGATCAACGTG | |
| | CCTAAAACAATGGAGTCCGGCACCTTCATCACCGACAAGACCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCA | |
| AT nucleotide | AACATCCAGAATCCCGAGCCTGCGGTGTACCAGCTGAAGGACCCCCGCTCT | 273 |
| sequence | CAGGATAGCACACTGTGCCTGTTCACCGACTTTGATAGCCAGATCAACGTG | |
| | CCTAAAACAATGGAGTCCGGCACCTTCATCACCGACAAGACCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCT | |
| AP15 nucleotide | AACATCCAGAATCCCGAGCCTGCGGTGTACCAGCTGAAGGACCCCCGCTCT | 276 |
| sequence | CAGGATAGCACACTGTGCCTGTTCACCGACTTTGATAGCCAGATCAACGTG | |
| | CCTAAAACAATGGAGTCCGGCACCTTCATCACCGACAAGACCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCAATGGATTGGACCTGGATT | |
| | CTGTTTCTGGTGGCCGCTGCCACAAGAGTGCACAGCAACTGGGTGAATGTG | |
| | ATCAGCGACCTGAAGAAGATCGAGGATCTGATCCAGAGCATGCACATTGAT | |
| | GCCACCCTGTACACAGAATCTGATGTGCACCCTAGCTGTAAAGTGACCGCC | |
| | ATGAAGTGTTTTCTGCTGGAGCTGCAGGTGATTTCTCTGGAAAGCGGAGAT | |
| | GCCTCTATCCACGACACAGTGGAGAATCTGATCATCCTGGCCAACAATAGC | |
| | CTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTG | |
| | CAGATGTTCATCAATACAAGCTCTGGCGGAGGATCTGGAGGAGGCGGATCT | |
| | GGAGGAGGAGGCAGTGGAGGCGGAGGATCTGGCGGAGGATCTCTGCAGATT | |
| | ACATGCCCTCCTCCAATGTCTGTGGAGCACGCCGATATTTGGGTGAAGTCC | |
| | TACAGCCTGTACAGCAGAGAGAGATACATCTGCAACAGCGGCTTTAAGAGA | |
| | AAGGCCGGCACCTCTTCTCTGACAGAGTGCGTGCTGAATAAGGCCACAAAT | |
| | GTGGCCCACTGGACAACACCTAGCCTGAAGTGCATTAGAGATCCTGCCCTG | |
| | GTCCACCAGAGGCCTGCCCCTCCATCTACAGTGACAACAGCCGGAGTGACA | |
| | CCTCAGCCTGAATCTCTGAGCCCTTCTGGAAAAGAACCTGCCGCCAGCTCT | |
| | CCTAGCTCTAATAATACCGCCGCCACAACAGCCGCCATTGTGCCTGGATCT | |
| | CAGCTGATGCCTAGCAAGTCTCCTAGCACAGGCACAACAGAGATCAGCAGC | |
| | CACGAATCTTCTCACGGAACACCTTCTCAGACCACCGCCAAGAATTGGGAG | |
| | CTGACAGCCTCTGCCTCTCACCAGCCTCCAGGAGTGTATCCTCAGGGCCAC | |
| | TCTGATACAACAGTGGCCATCAGCACATCTACAGTGCTGCTGTGTGGACTG | |
| | TCTGCCGTGTCTCTGCTGGCCTGTTACCTGAAGTCTAGACAGACACCTCCT | |
| | CTGGCCTCTGTGGAGATGGAGGCCATGGAAGCCCTGCCTGTGACATGGGGA | |
| | ACAAGCAGCAGAGATGAAGACCTGGAGAATTGTTCTCACCACCTG | |
| AP15T nucleotide | AACATCCAGAATCCCGAGCCTGCGGTGTACCAGCTGAAGGACCCCCGCTCT | 278 |
| sequence | CAGGATAGCACACTGTGCCTGTTCACCGACTTTGATAGCCAGATCAACGTG | |
| | CCTAAAACAATGGAGTCCGGCACCTTCATCACCGACAAGACCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGCGACCAATTTCAGCCTGCTGAAG | |
| | CAGGCGGGCGATGTGGAGGAGAACCCTGGCCCAATGGATTGGACCTGGATT | |
| | CTGTTTCTGGTGGCCGCTGCCACAAGAGTGCACAGCAACTGGGTGAATGTG | |
| | ATCAGCGACCTGAAGAAGATCGAGGATCTGATCCAGAGCATGCACATTGAT | |
| | GCCACCCTGTACACAGAATCTGATGTGCACCCTAGCTGTAAAGTGACCGCC | |
| | ATGAAGTGTTTTCTGCTGGAGCTGCAGGTGATTTCTCTGGAAAGCGGAGAT | |
| | GCCTCTATCCACGACACAGTGGAGAATCTGATCATCCTGGCCAACAATAGC | |
| | CTGAGCAGCAATGGCAATGTGACAGAGTCTGGCTGTAAGGAGTGTGAGGAG | |
| | CTGGAGGAGAAGAACATCAAGGAGTTTCTGCAGAGCTTTGTGCACATCGTG | |
| | CAGATGTTCATCAATACAAGCTCTGGCGGAGGATCTGGAGGAGGCGGATCT | |
| | GGAGGAGGAGGCAGTGGAGGCGGAGGATCTGGCGGAGGATCTCTGCAGATT | |
| | ACATGCCCTCCTCCAATGTCTGTGGAGCACGCCGATATTTGGGTGAAGTCC | |
| | TACAGCCTGTACAGCAGAGAGAGATACATCTGCAACAGCGGCTTTAAGAGA | |
| | AAGGCCGGCACCTCTTCTCTGACAGAGTGCGTGCTGAATAAGGCCACAAAT | |
| | GTGGCCCACTGGACAACACCTAGCCTGAAGTGCATTAGAGATCCTGCCCTG | |
| | GTCCACCAGAGGCCTGCCCCTCCATCTACAGTGACAACAGCCGGAGTGACA | |
| | CCTCAGCCTGAATCTCTGAGCCCTTCTGGAAAAGAACCTGCCGCCAGCTCT | |
| | CCTAGCTCTAATAATACCGCCGCCACAACAGCCGCCATTGTGCCTGGATCT | |
| | CAGCTGATGCCTAGCAAGTCTCCTAGCACAGGCACAACAGAGATCAGCAGC | |
| | CACGAATCTTCTCACGGAACACCTTCTCAGACCACCGCCAAGAATTGGGAG | |
| | CTGACAGCCTCTGCCTCTCACCAGCCTCCAGGAGTGTATCCTCAGGGCCAC | |
| | TCTGATACAACAGTGGCCATCAGCACATCTACAGTGCTGCTGTGTGGACTG | |
| | TCTGCCGTGTCTCTGCTGGCCTGTTACCTGAAGTCTAGACAGACACCTCCT | |
| | CTGGCCTCTGTGGAGATGGAGGCCATGGAAGCCCTGCCTGTGACATGGGGA | |
| | ACAAGCAGCAGAGATGAAGACCTGGAGAATTGTTCTCACCACCTGCGGGCG | |
| | AAACGCTCTGGAAGCGGAGAGGGCAGAGGAAGTCTTCTAACATGCGGTGAC | |
| | GTGGAGGAGAATCCCGGCCCT | |
| AT15 nucleotide | AACATCCAGAATCCCGAGCCTGCGGTGTACCAGCTGAAGGACCCCCGCTCT | 279 |
| sequence | CAGGATAGCACACTGTGCCTGTTCACCGACTTTGATAGCCAGATCAACGTG | |
| | CCTAAAACAATGGAGTCCGGCACCTTCATCACCGACAAGACCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCTATGGATTGGACCTGGATTCTG | |
| | TTTCTGGTGGCCGCTGCCACAAGAGTGCACAGCAACTGGGTGAATGTGATC | |
| | AGCGACCTGAAGAAGATCGAGGATCTGATCCAGAGCATGCACATTGATGCC | |
| | ACCCTGTACACAGAATCTGATGTGCACCCTAGCTGTAAAGTGACCGCCATG | |
| | AAGTGTTTTCTGCTGGAGCTGCAGGTGATTTCTCTGGAAAGCGGAGATGCC | |
| | TCTATCCACGACACAGTGGAGAATCTGATCATCCTGGCCAACAATAGCCTG | |
| | AGCAGCAATGGCAATGTGACAGAGTCTGGCTGTAAGGAGTGTGAGGAGCTG | |
| | GAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGA | |
| | GGAGGAGGCAGTGGAGGCGGAGGATCTGGCGGAGGATCTCTGCAGATTACA | |
| | TGCCCTCCTCCAATGTCTGTGGAGCACGCCGATATTTGGGTGAAGTCCTAC | |
| | AGCCTGTACAGCAGAGAGAGATACATCTGCAACAGCGGCTTTAAGAGAAAG | |
| | GCCGGCACCTCTTCTCTGACAGAGTGCGTGCTGAATAAGGCCACAAATGTG | |
| | GCCCACTGGACAACACCTAGCCTGAAGTGCATTAGAGATCCTGCCCTGGTC | |
| | CACCAGAGGCCTGCCCCTCCATCTACAGTGACAACAGCCGGAGTGACACCT | |
| | CAGCCTGAATCTCTGAGCCCTTCTGGAAAAGAACCTGCCGCCAGCTCTCCT | |
| | AGCTCTAATAATACCGCCGCCACAACAGCCGCCATTGTGCCTGGATCTCAG | |
| | CTGATGCCTAGCAAGTCTCCTAGCACAGGCACAACAGAGATCAGCAGCCAC | |
| | GAATCTTCTCACGGAACACCTTCTCAGACCACCGCCAAGAATTGGGAGCTG | |
| | ACAGCCTCTGCCTCTCACCAGCCTCCAGGAGTGTATCCTCAGGGCCACTCT | |
| | GATACAACAGTGGCCATCAGCACATCTACAGTGCTGCTGTGTGGACTGTCT | |
| | GCCGTGTCTCTGCTGGCCTGTTACCTGAAGTCTAGACAGACACCTCCTCTG | |
| | GCCTCTGTGGAGATGGAGGCCATGGAAGCCCTGCCTGTGACATGGGGAACA | |
| | AGCAGCAGAGATGAAGACCTGGAGAATTGTTCTCACCACCTG | |
| AT15P nucleotide | AACATCCAGAATCCCGAGCCTGCGGTGTACCAGCTGAAGGACCCCCGCTCT | 281 |
| sequence | CAGGATAGCACACTGTGCCTGTTCACCGACTTTGATAGCCAGATCAACGTG | |
| | CCTAAAACAATGGAGTCCGGCACCTTCATCACCGACAAGACCGTGCTGGAT | |
| | ATGAAAGCGATGGACTCCAAGTCTAACGGCGCGATCGCGTGGTCCAATCAG | |
| | ACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACA | |
| | GACATGAACCTGAATTTTCAGAATCTGCTGGTCATCGTGCTGAGAATCCTG | |
| | CTGCTGAAGGTGGCGGGCTTTAATCTGCTGATGACACTGCGGCTGTGGAGT | |
| | TCCCGGGCGAAACGCTCTGGAAGCGGAGAGGGCAGAGGAAGTCTTCTAACA | |
| | TGCGGTGACGTGGAGGAGAATCCCGGCCCTATGGATTGGACCTGGATTCTG | |
| | TTTCTGGTGGCCGCTGCCACAAGAGTGCACAGCAACTGGGTGAATGTGATC | |
| | AGCGACCTGAAGAAGATCGAGGATCTGATCCAGAGCATGCACATTGATGCC | |
| | ACCCTGTACACAGAATCTGATGTGCACCCTAGCTGTAAAGTGACCGCCATG | |
| | AAGTGTTTTCTGCTGGAGCTGCAGGTGATTTCTCTGGAAAGCGGAGATGCC | |
| | TCTATCCACGACACAGTGGAGAATCTGATCATCCTGGCCAACAATAGCCTG | |
| | AGCAGCAATGGCAATGTGACAGAGTCTGGCTGTAAGGAGTGTGAGGAGCTG | |
| | GAGGAGAAGAACATCAAGGAGTTTCTGCAGAGCTTTGTGCACATCGTGCAG | |
| | ATGTTCATCAATACAAGCTCTGGCGGAGGATCTGGAGGAGGCGGATCTGGA | |
| | GGAGGAGGCAGTGGAGGCGGAGGATCTGGCGGAGGATCTCTGCAGATTACA | |
| | TGCCCTCCTCCAATGTCTGTGGAGCACGCCGATATTTGGGTGAAGTCCTAC | |
| | AGCCTGTACAGCAGAGAGAGATACATCTGCAACAGCGGCTTTAAGAGAAAG | |
| | GCCGGCACCTCTTCTCTGACAGAGTGCGTGCTGAATAAGGCCACAAATGTG | |
| | GCCCACTGGACAACACCTAGCCTGAAGTGCATTAGAGATCCTGCCCTGGTC | |
| | CACCAGAGGCCTGCCCCTCCATCTACAGTGACAACAGCCGGAGTGACACCT | |
| | CAGCCTGAATCTCTGAGCCCTTCTGGAAAAGAACCTGCCGCCAGCTCTCCT | |
| | AGCTCTAATAATACCGCCGCCACAACAGCCGCCATTGTGCCTGGATCTCAG | |
| | CTGATGCCTAGCAAGTCTCCTAGCACAGGCACAACAGAGATCAGCAGCCAC | |
| | GAATCTTCTCACGGAACACCTTCTCAGACCACCGCCAAGAATTGGGAGCTG | |
| | ACAGCCTCTGCCTCTCACCAGCCTCCAGGAGTGTATCCTCAGGGCCACTCT | |
| | GATACAACAGTGGCCATCAGCACATCTACAGTGCTGCTGTGTGGACTGTCT | |
| | GCCGTGTCTCTGCTGGCCTGTTACCTGAAGTCTAGACAGACACCTCCTCTG | |
| | GCCTCTGTGGAGATGGAGGCCATGGAAGCCCTGCCTGTGACATGGGGAACA | |
| | AGCAGCAGAGATGAAGACCTGGAGAATTGTTCTCACCACCTGCGGGCGAAA | |
| | CGCTCTGGAAGCGGAGCGACCAATTTCAGCCTGCTGAAGCAGGCGGGCGAT | |
| | GTGGAGGAGAACCCTGGCCCA | |

### 1.6 Transposon and Transposase Systems

In some embodiments, transgenes of the recombinant vector are introduced into an immune effector cell via synthetic DNA transposable elements, e.g., a DNA transposon/transposase system, e.g., Sleeping Beauty (SB). SB belongs to the Tc1/mariner superfamily of DNA transposons. DNA transposons translocate from one DNA site to another in a simple, cut-and-paste manner. Transposition is a precise process in which a defined DNA segment is excised from one DNA molecule and moved to another site in the same or different DNA molecule or genome.

Exemplary DNA transposon/transposase systems include, but are not limited to, Sleeping Beauty (see, e.g., U.S. Pat. Nos. 6,489,458, 8,227,432, the contents of each of which are incorporated by reference in their entirety herein), piggyBac transposon system (see e.g., U.S. Pat. No. 9,228,180, Wilson et al, “PiggyBac Transposon-mediated Gene Transfer in Human Cells,” Molecular Therapy, 15:139-145 (2007), the contents of each of which are incorporated by reference in their entirety herein), piggyBac transposon system (see e.g., Mitra et al., “Functional characterization of piggyBac from the bat Myotis lucifugus unveils an active mammalian DNA transposon,” Proc. Natl. Acad. Sci USA 110:234-239 (2013), the contents of which are incorporated by reference in their entirety herein), TcBuster (see e.g., Woodard et al. “Comparative Analysis of the Recently Discovered hAT Transposon TcBuster in Human Cells,” PLOS ONE, 7(11): e42666 (November 2012), the contents of which are incorporated by reference in their entirety herein), and the Tol2 transposon system (see e.g., Kawakami, “Tol2: a versatile gene transfer vector in vertebrates,” Genome Biol. 2007; 8(Suppl 1): S7, the contents of each of which are incorporated by reference in their entirety herein). Additional exemplary transposon/transposase systems are provided in U.S. Pat. Nos. 7,148,203; 8,227,432; US20110117072; Mates et al., Nat Genet, 41(6):753-61 (2009); and Ivies et al., Cell, 91(4):501-10, (1997), the contents of each of which are incorporated by reference in their entirety herein).

In some embodiments, the transgenes described herein are introduced into an immune effector cell via the SB transposon/transposase system. The SB transposon system comprises a SB a transposase and SB transposon(s). The SB transposon system can comprise a naturally occurring SB transposase or a derivative, variant, and/or fragment that retains activity, and a naturally occurring SB transposon, or a derivative, variant, and/or fragment that retains activity. An exemplary SB system is described in, Hackett et al., “A Transposon and Transposase System for Human Application,” Mol Ther 18:674-83, (2010), the entire contents of which are incorporated by reference herein.

In some embodiments, the vector comprises a Left inverted terminal repeat (ITR), i.e., an ITR that is 5′ to an expression cassette, and a Right ITR, i.e., an ITR that is 3′ to an expression cassette. The Left ITR and Right ITR flank the polycistronic expression cassette of the vector. In some embodiments, the Left ITR is in reverse orientation relative to the polycistronic expression cassette, and the Right ITR is in the same orientation relative to the polycistronic expression cassette. In some embodiments, the Right ITR is in reverse orientation relative to the polycistronic expression cassette, and the Left ITR is in the same orientation relative to the polycistronic expression cassette.

In some embodiments, the Left ITR and the Right ITR are ITRs of a DNA transposon selected from the group consisting of a Sleeping Beauty transposon, a piggyBac transposon, TcBuster transposon, and a Tol2 transposon. In some embodiments, the Left ITR and the Right ITR are ITRs of the Sleeping Beauty DNA transposon.

In some embodiments, the Left ITR comprises a polynucleotide sequence at least 75%, 80%, 85%, 90%, 95%, 96%, 97%, 98%, 99%, or 100% identical to the polynucleotide sequence of SEQ ID NO: 290 or 291. In some embodiments, the Left ITR comprises a polynucleotide sequence at least 75%, 80%, 85%, 90%, 95%, 96%, 97%, 98%, 99%, or 100% identical to the polynucleotide sequence of SEQ ID NO: 290. In some embodiments, the Left ITR comprises the polynucleotide sequence of SEQ ID NO: 290. In some embodiments, the Left ITR comprises a polynucleotide sequence at least 75%, 80%, 85%, 90%, 95%, 96%, 97%, 98%, 99%, or 100% identical to the polynucleotide sequence of SEQ ID NO: 291. In some embodiments, the Left ITR comprises the polynucleotide sequence of SEQ ID NO: 291. In some embodiments, the Right ITR comprises a polynucleotide sequence at least 75%, 80%, 85%, 90%, 95%, 96%, 97%, 98%, 99%, or 100% identical to the polynucleotide sequence of SEQ ID NO: 292, 293, or 294. In some embodiments, the Right ITR comprises a polynucleotide sequence at least 75%, 80%, 85%, 90%, 95%, 96%, 97%, 98%, 99%, or 100% identical to the polynucleotide sequence of SEQ ID NO: 292. In some embodiments, the Right ITR comprises a polynucleotide sequence at least 75%, 80%, 85%, 90%, 95%, 96%, 97%, 98%, 99%, or 100% identical to the polynucleotide sequence of SEQ ID NO: 293. In some embodiments, the Right ITR comprises a polynucleotide sequence at least 75%, 80%, 85%, 90%, 95%, 96%, 97%, 98%, 99%, or 100% identical to the polynucleotide sequence of SEQ ID NO: 294. In some embodiments, the Right ITR comprises the polynucleotide sequence of SEQ ID NO: 292. In some embodiments, the Right ITR comprises the polynucleotide sequence of SEQ ID NO: 293. In some embodiments, the Right ITR comprises the polynucleotide sequence of SEQ ID NO: 294.

The polynucleotide sequences of exemplary SB ITRs are provided in Table 12, herein.

TABLE 12
Polynucleotide sequence of exemplary SB ITRs.

| Description | Polynucleotide Sequence | SEQ ID NO |
|---|---|---|
| Left ITR-A | AGTTGAAGTCGGAAGTTTACATACACTTAAGTTGGAGTCATTAAAA | 290 |
| | CTCGTTTTTCAACTACTCCACAAATTTCTTGTTAACAAACAATAGT | |
| | TTTGGCAAGTCAGTTAGGACATCTACTTTGTGCATGACACAAGTCA | |
| | TTTTTCCAACAATTGTTTACAGACAGATTATTTCACTTATAATTCA | |
| | CTGTATCACAATTCCAGTGGGTCAGAAGTTTACATACACTAA | |
| Left ITR-A2 | TACAGTTGAAGTCGGAAGTTTACATACACTTAAGTTGGAGTCATTA | 295 |
| | AAACTCGTTTTTCAACTACTCCACAAATTTCTTGTTAACAAACAAT | |
| | AGTTTTGGCAAGTCAGTTAGGACATCTACTTTGTGCATGACACAAG | |
| | TCATTTTTCCAACAATTGTTTACAGACAGATTATTTCACTTATAAT | |
| | TCACTGTATCACAATTCCAGTGGGTCAGAAGTTTACATACACTAA | |
| Left ITR-B | ATATCAATTGAGTTGAAGTCGGAAGTTTACATACACTTAAGTTGGA | 291 |
| | GTCATTAAAACTCGTTTTTCAACTACACCACAAATTTCTTGTTAAC | |
| | AAACAATAGTTTTGGCAAGTCAGTTAGGACATCTACTTTGTGCATG | |
| | ACACAAGTCATTTTTCCAACAATTGTTTACAGACAGATTATTTCAC | |
| | TTATAATTCACTGTATCACAATTCCAGTGGGTCAGAAGTTTACATA | |
| | CACTAACAATTGATAT | |
| Right ITR-A | TTGAGTGTATGTAAACTTCTGACCCACTGGGAATGTGATGAAAGAA | 292 |
| | ATAAAAGCTGAAATGAATCATTCTCTCTACTATTATTCTGATATTT | |
| | CACATTCTTAAAATAAAGTGGTGATCCTAACTGACCTAAGACAGGG | |
| | AATTTTTACTAGGATTAAATGTCAGGAATTGTGAAAAAGTGAGTTT | |
| | AAATGTATTTGGCTAAGGTGTATGTAAACTTCCGACTTCAACTG | |
| Right ITR-A2 | TTGAGTGTATGTAAACTTCTGACCCACTGGGAATGTGATGAAAGAA | 296 |
| | ATAAAAGCTGAAATGAATCATTCTCTCTACTATTATTCTGATATTT | |
| | CACATTCTTAAAATAAAGTGGTGATCCTAACTGACCTAAGACAGGG | |
| | AATTTTTACTAGGATTAAATGTCAGGAATTGTGAAAAAGTGAGTTT | |
| | AAATGTATTTGGCTAAGGTGTATGTAAACTTCCGACTTCAACTGTA | |
| Right ITR-B | TTGAGTGTATGTTAACTTCTGACCCACTGGGAATGTGATGAAAGAA | 293 |
| | ATAAAAGCTGAAATGAATCATTCTCTCTACTATTATTCTGATATTT | |
| | CACATTCTTAAAATAAAGTGGTGATCCTAACTGACCTTAAGACAGG | |
| | GAATCTTTACTCGGATTAAATGTCAGGAATTGTGAAAAAGTGAGTT | |
| | TAAATGTATTTGGCTAAGGTGTATGTAAACTTCCGACTTCAACT | |
| Right ITR-B2 | TTGAGTGTATGTTAACTTCTGACCCACTGGGAATGTGATGAAAGAA | 297 |
| | ATAAAAGCTGAAATGAATCATTCTCTCTACTATTATTCTGATATTT | |
| | CACATTCTTAAAATAAAGTGGTGATCCTAACTGACCTTAAGACAGG | |
| | GAATCTTTACTCGGATTAAATGTCAGGAATTGTGAAAAAGTGAGTT | |
| | TAAATGTATTTGGCTAAGGTGTATGTAAACTTCCGACTTCAACTGT | |
| | A | |
| Right ITR-C | ATATCTCGAGTTGAGTGTATGTTAACTTCTGACCCACTGGGAATGT | 294 |
| | GATGAAAGAAATAAAAGCTGAAATGAATCATTCTCTCTACTATTAT | |
| | TCTGATATTTCACATTCTTAAAATAAAGTGGTGATCCTAACTGACC | |
| | TTAAGACAGGGAATCTTTACTCGGATTAAATGTCAGGAATTGTGAA | |
| | AAAGTGAGTTTAAATGTATTTGGCTAAGGTGTATGTAAACTTCCGA | |
| | CTTCAACTCTCGAGATAT | |

In some embodiments, the DNA transposase is a SB transposase. In some embodiments, the SB transposase is selected from the group consisting of SB 11, SB 100X, hSB110, and hSB81. In some embodiments, the SB transposase is SB 11. Exemplary SB transposases are described in U.S. Pat. No. 9,840,696, US20160264949, U.S. Pat. No. 9,228,180, WO2019038197, U.S. Ser. No. 10/174,309, and U.S. Ser. No. 10/570,382, the full contents of each of which is incorporated by reference herein.

In some embodiments, the DNA transposase comprises an amino acid sequence at least 95%, 96%, 97%, 98%, 99% or 100% identical to the amino acid sequence of SEQ ID NO: 300. In some embodiments, the DNA transposase comprises the amino acid sequence of SEQ ID NO: 300. In some embodiments, the amino acid sequence of the DNA transposase consists of a sequence at least 95%, 96%, 97%, 98%, 99% or 100% identical to the amino acid sequence of SEQ ID NO: 300. In some embodiments, the amino acid sequence of the DNA transposase consists of the amino acid sequence of SEQ ID NO: 300.

In some embodiments, the DNA transposase comprises an amino acid sequence that lacks its N-terminal methionine. In some embodiments, the DNA transposase comprises an amino acid sequence at least 95%, 96%, 97%, 98%, 99% or 100% identical to the amino acid sequence of SEQ ID NO: 300 lacking its N-terminal methionine, i.e., amino acids 2-340 of SEQ ID NO:300. In some embodiments, the DNA transposase comprises the amino acid sequence of SEQ ID NO: 300 lacking its N-terminal methionine, i.e., amino acids 2-340 of SEQ ID NO:300. In some embodiments, the amino acid sequence of the DNA transposase consists of a sequence at least 95%, 96%, 97%, 98%, 99% or 100% identical to the amino acid sequence of SEQ ID NO: 300 lacking its N-terminal methionine, i.e., amino acids 2-340 of SEQ ID NO:300. In some embodiments, the amino acid sequence of the DNA transposase consists of the amino acid sequence of SEQ ID NO: 300 lacking its N-terminal methionine, i.e., amino acids 2-340 of SEQ ID NO:300.

In some embodiments, the DNA transposase is encoded by a polynucleotide sequence at least 75%, 80%, 85%, 90%, 95%, 96%, 97%, 98%, 99%, or 100% identical to the polynucleotide sequence of SEQ ID NO: 301. In some embodiments, the DNA transposase is encoded by the polynucleotide sequence of SEQ ID NO: 301.

In some embodiments, the DNA transposase is encoded by a polynucleotide that is introduced into a cell. In some embodiments, the polynucleotide encoding the DNA transposase is a DNA vector. In some embodiments, the polynucleotide encoding the DNA transposase is an RNA vector. In some embodiments, the DNA transposase is encoded on a first vector and the transgenes are encoded on a second vector. In some embodiments, the DNA transposase is directly introduced to a population of cells as a polypeptide.

The amino acid and polynucleotide sequence of an exemplary SB transposase is provided in Table 13, herein.

TABLE 13
Amino acid and polynucleotide sequence of an exemplary SB transposase.

| Description | Sequence | SEQ ID NO |
|---|---|---|
| SB11 (exemplary | MGKSKEISQDLRKKIVDLHKSGSSLGAISKRLKVPRSSVQTIVRKYKH | 300 |
| amino acid | HGTTQPSYRSGRRRVLSPRDERTLVRKVQINPRTTAKDLVKMLEETGT | |
| sequence) | KVSISTVKRVLYRHNLKGRSARKKPLLQNRHKKARLRFARAHGDKDRT | |
| | FWRNVLWSDETKIELFGHNDHRYVWRKKGEACKPKNTIPTVKHGGGSI | |
| | MLWGCFAAGGTGALHKIDGIMRKENYVDILKQHLKTSVRKLKLGRKWV | |
| | FQQDNDPKHTSKHVRKWLKDNKVKVLEWPSQSPDLNPIENLWAELKKR | |
| | VRARRPTNLTQLHQLCQEEWAKIHPTYCGKLVEGYPKRLTQVKQFKGN | |
| | ATKY | |
| SB11 (exemplary | ATGGGAAAATCAAAAGAAATCAGCCAAGACCTCAGAAAAAAAATTGTA | 301 |
| nucleotide | GACCTCCACAAGTCTGGTTCATCCTTGGGAGCAATTTCCAAACGCCTG | |
| sequence) | AAAGTACCACGTTCATCTGTACAAACAATAGTACGCAAGTATAAACAC | |
| | CATGGGACCACGCAGCCGTCATACCGCTCAGGAAGGAGACGCGTTCTG | |
| | TCTCCTAGAGATGAACGTACTTTGGTGCGAAAAGTGCAAATCAATCCC | |
| | AGAACAACAGCAAAGGACCTTGTGAAGATGCTGGAGGAAACAGGTACA | |
| | AAAGTATCTATATCCACAGTAAAACGAGTCCTATATCGACATAACCTG | |
| | AAAGGCCGCTCAGCAAGGAAGAAGCCACTGCTCCAAAACCGACATAAG | |
| | AAAGCCAGACTACGGTTTGCAAGAGCACATGGGGACAAAGATCGTACT | |
| | TTTTGGAGAAATGTCCTCTGGTCTGATGAAACAAAAATAGAACTGTTT | |
| | GGCCATAATGACCATCGTTATGTTTGGAGGAAGAAGGGGGAGGCTTGC | |
| | AAGCCGAAGAACACCATCCCAACCGTGAAGCACGGGGGTGGCAGCATC | |
| | ATGTTGTGGGGGTGCTTTGCTGCAGGAGGGACTGGTGCACTTCACAAA | |
| | ATAGATGGCATCATGAGGAAGGAAAATTATGTGGATATATTGAAGCAA | |
| | CATCTCAAGACATCAGTCAGGAAGTTAAAGCTTGGTCGCAAATGGGTC | |
| | TTCCAACAAGACAATGACCCCAAGCATACTTCCAAACACGTGAGAAAA | |
| | TGGCTTAAGGACAACAAAGTCAAGGTATTGGAGTGGCCATCACAAAGC | |
| | CCTGACCTCAATCCTATAGAAAATTTGTGGGCAGAACTGAAAAAGCGT | |
| | GTGCGAGCAAGGAGGCCTACAAACCTGACTCAGTTACACCAGCTCTGT | |
| | CAGGAGGAATGGGCCAAAATTCACCCAACTTATTGTGGGAAGCTTGTG | |
| | GAAGGCTACCCGAAACGTTTGACCCAAGTTAAACAATTTAAAGGCAAT | |
| | GCTACCAAATAC | |

### 1.7 Overview of Expansion Methods

In certain embodiments of expanding electroporated T cells, a multi-step process is employed. The multi-step process includes at least one expansion protocol, preceded by a separate pre-expansion recovery step.

In one embodiment, a multi-step manufacture process for electroporated T cells begins with a pre-expansion recovery step occurring immediately after electroporation of the T cells with a nucleic acid, such as a plasmid containing one of the polycistronic vectors described herein. Typically, a pre-expansion recovery step comprises incubation of the electroporated T cells in a first culture medium (also referred to herein as recovery medium) for a period of time sufficient for recovery of the electroporated T cells. In some embodiments, the electroporated T cells are incubated in the first culture medium for 24 hours or less. In some embodiments, the electroporated T cells are incubated in the first culture medium overnight. In some embodiments, the electroporated T cells are incubated in the first culture medium for 18 hours or less, for 12 hours or less, for 11 hours or less, for 10 hours or less, for 9 hours or less, for 8 hours or less, for 7 hours or less, for 6 hours or less, for 5 hours or less, for 4 hours or less, for 3 hours or less, for 2 hours or less, or for 1 hour or less.

In some cases, the recovery phase can be performed in a gas permeable container using methods known in the art. For example, T cells can be rapidly expanded using non-specific T-cell receptor stimulation in the presence of one or more T cell-stimulating cytokines selected from IL-2, IL-7, IL-15, IL-21, and combinations thereof as well as an exogenous glutathione precursor. In some embodiments, the exogenous glutathione precursor is NAC. In some embodiments, the culture components of the first culture medium comprise IL-15 and an exogenous glutathione precursor. In some embodiments, the culture components of the first culture medium also comprise IL-7. In some embodiments, the culture components of the first culture medium also comprise IL-21 In some embodiments, the culture components of the first culture medium also comprise IL-2. In some embodiments, the exogenous glutathione precursor is NAC. In some embodiments, the culture components of the first culture medium comprise IL-7, IL-15 and NAC. In some embodiments, the culture components of the first culture medium comprise IL-2 and IL-21.

In multi-step electroporated T cell manufacture, the electroporated T cell population is expanded in number after pre-expansion recovery. This expansion may include multiple expansion phases, referred to as first expansion phase, second expansion phase, third expansion phase, etc. The expansion protocol is generally accomplished using a culture media comprising a number of components, including a cytokine source and in some embodiments, an exogenous glutathione precursor, in a gas-permeable container. In some embodiments, the exogenous glutathione precursor is NAC. In some cases, the expansion phase(s) can be performed using any flasks or containers known by those of skill in the art and can proceed for 7-14 days or longer. In some cases, the expansion phase(s) are performed in a closed system bioreactor, such as G-REX-10 or a G-REX-100.

In some cases, the expansion phase(s) can be performed using non-specific T-cell receptor stimulus that can include, for example, an anti-CD3 antibody, such as about 30 ng/ml of OKT3, a mouse monoclonal anti-CD3 antibody (commercially available from Ortho-McNeil, Raritan, N.J. or Miltenyi Biotech, Auburn, Calif.) or UCHT-1 (commercially available from BioLegend, San Diego, Calif., USA). In some cases, the expansion phase(s) can be conducted in a supplemented cell culture medium comprising one or more T cell-stimulating cytokines IL-2, IL-7, IL-12, IL-15, IL-21, and combinations thereof, OKT-3, and antigen-presenting feeder cells. In some cases, the antigen-presenting feeder cells (APCs) are PBMCs (peripheral blood mononuclear cells). In some cases, the ratio of T cells to PBMCs and/or antigen-presenting cells in the expansion phase(s) is 1 to 25 and 1 to 500. In some cases, the expansion phase(s) are performed in flasks with the bulk of T cells being mixed with a 100- or 200-fold excess of inactivated feeder cells, about 30 mg/ml OKT3 anti-CD3 antibody and, in some embodiments, about 3000 U/ml IL-2 in 150 ml media. Media replacement is done (generally ½ or ¾ media replacement via respiration with fresh media) until the cells are transferred to an alternative growth chamber. Alternative growth chambers include G-REX flasks and other gas permeable containers.

In many cases, the feeder cells used in T cell expansion phase(s) are peripheral blood mononuclear cells (PBMCs) obtained from standard whole blood units from healthy blood donors. The PBMCs are obtained using standard methods such as FICOLL-Paque gradient separation. In general, allogeneic PBMCs are inactivated, either via irradiation or heat treatment, and used in the expansion phase(s). In some cases, PBMCs are considered replication incompetent and accepted for use in expansion phase(s) if the total number of viable cells after 14 days of culture is less than the initial viable cell number put into culture on day 0.

In some cases, PBMCs are considered replication incompetent and accepted for use in the T cell expansion phase(s) described herein if the total number of viable cells, cultured in the presence of OKT3 and IL-2, on day 7 and day 14 has not increased from the initial viable cell number put into culture on day 0 of the relevant expansion phase. In some cases, the PBMCs are cultured in the presence of about 30 ng/ml OKT3 antibody and, in some embodiments, 3000 U/ml IL-2. In some cases, the expansion phases(s) require a ratio of about 2.5×10⁹ feeder cells to between 12.5×10⁶ T cells and 100×10⁶ T cells.

In some embodiments, the feeder cells express a TCR agonist. In some embodiments, the feeder cells express an agonist of a T cell costimulatory molecule. In some embodiments, the TCR agonist and/or agonist of a T cell costimulatory molecule are expressed on the surface of the feeder cells.

In one embodiment, the agonist of a T cell costimulatory molecule is a CD28 agonist. In one embodiment, the agonist of a T cell costimulatory molecule is a CD137 (i.e., 4-1BB) agonist. In one embodiment, the agonist of a T cell costimulatory molecule is a CD2 agonist. In some embodiments, a 4-1BB ligand is expressed on the surface of the feeder cells.

In another aspect, the present disclosure provides methods for expanding a population of T cells comprising culturing the population of T cells in a culture medium comprising a nanomatrix comprising a colloidal suspension of matrices of polymer chains, wherein the matrices are attached to TCR agonists and agonists of a T cell costimulatory molecule, wherein each matrix is 1 to 500 nm in length in its largest dimension.

In some embodiments, the TCR agonist and/or the CD28 agonist are linked to a nanomatrix comprising a colloidal suspension of matrices of polymer chains, wherein each nanomatrix is 1 to 500 nm in length in its largest dimension. In some embodiments, the nanomatrix is 1 to 50 nm in length in its largest dimension. In some embodiments, the nanomatrix is 50 to 100 nm in length in its largest dimension. In some embodiments, the nanomatrix is 100 to 150 nm in length in its largest dimension. In some embodiments, the nanomatrix is 150 to 200 nm in length in its largest dimension. In some embodiments, the nanomatrix is 200 to 250 nm in length in its largest dimension. In some embodiments, the nanomatrix is 250 to 300 nm in length in its largest dimension. In some embodiments, the nanomatrix is 300 to 350 nm in length in its largest dimension. In some embodiments, the nanomatrix is 350 to 400 nm in length in its largest dimension. In some embodiments, the nanomatrix is 400 to 450 nm in length in its largest dimension. In some embodiments, the nanomatrix is 450 to 500 nm in length in its largest dimension.

In some embodiments, the TCR agonists and agonists of a T cell costimulatory molecule utilized in the described methods are attached to the same polymer chains. In some embodiments, the TCR agonists and agonists of a T cell costimulatory molecule are attached to different polymer chains. In some embodiments, the TCR agonists are attached to the matrices at 25 μg per mg of matrix. In some embodiments, the agonist of a T cell costimulatory molecule is attached to the matrices at 25 μg per mg of matrix. Typically, the agonists are covalently attached to the polymer chains that comprise the matrices within the nanomatrix.

In some embodiments, the TCR agonist and the CD28 agonist are attached to the same polymer chains. In some embodiments, the TCR agonist and the CD28 agonist are attached to different polymer chains. In some embodiments, the TCR agonist, or fragment thereof, is attached to the nanomatrix at 25 μg per mg of nanomatrix. In some embodiments, the TCR agonist, or fragment thereof, is attached to the nanomatrix at about 5 μg to about 10 μg per mg of nanomatrix. In some embodiments, the TCR agonist, or fragment thereof, is attached to the nanomatrix at about 10 μg to about 15 μg per mg of nanomatrix. In some embodiments, the TCR agonist, or fragment thereof, is attached to the nanomatrix at about 15 μg to about 20 μg per mg of nanomatrix. In some embodiments, the TCR agonist, or fragment thereof, is attached to the nanomatrix at about 20 μg to about 25 μg per mg of nanomatrix. In some embodiments, the TCR agonist, or fragment thereof, is attached to the nanomatrix at about 25 μg to about 30 μg per mg of nanomatrix. In some embodiments, the TCR agonist, or fragment thereof, is attached to the nanomatrix at about 30 μg to about 35 μg per mg of nanomatrix. In some embodiments, the TCR agonist, or fragment thereof, is attached to the nanomatrix at about 35 μg to about 40 μg per mg of nanomatrix. In some embodiments, the TCR agonist, or fragment thereof, is attached to the nanomatrix at about 40 μg to about 45 μg per mg of nanomatrix. In some embodiments, the TCR agonist, or fragment thereof, is attached to the nanomatrix at about 45 mg to about 50 mg per mg of nanomatrix. In some embodiments, the TCR agonist is a CD3 agonist.

In some embodiments, the CD28 agonist, or fragment thereof, is attached to the nanomatrix at 25 mg per mg of nanomatrix. In some embodiments, the CD28 agonist, or fragment thereof, is attached to the nanomatrix at about 5 μg to about 10 μg per mg of nanomatrix. In some embodiments, the CD28 agonist, or fragment thereof, is attached to the nanomatrix at about 10 μg to about 15 μg per mg of nanomatrix. In some embodiments, the CD28 agonist, or fragment thereof, is attached to the nanomatrix at about 15 μg to about 20 μg per mg of nanomatrix. In some embodiments, the CD28 agonist, or fragment thereof, is attached to the nanomatrix at about 20 μg to about 25 μg per mg of nanomatrix. In some embodiments, the CD28 agonist, or fragment thereof, is attached to the nanomatrix at about 25 μg to about 30 μg per mg of nanomatrix. In some embodiments, the CD28 agonist, or fragment thereof, is attached to the nanomatrix at about 30 μg to about 35 μg per mg of nanomatrix. In some embodiments, the CD28 agonist, or fragment thereof, is attached to the nanomatrix at about 35 μg to about 40 μg per mg of nanomatrix. In some embodiments, the CD28 agonist, or fragment thereof, is attached to the nanomatrix at about 40 μg to about 45 μg per mg of nanomatrix. In some embodiments, the CD28 agonist, or fragment thereof, is attached to the nanomatrix at about 45 μg to about 50 μg per mg of nanomatrix.

In some embodiments, the ratio of volume of nanomatrix to volume of T cells is greater than or equal to 1:5. In some embodiments, the ratio of volume of nanomatrix to volume of T cells is greater than or equal to 1:10. In some embodiments, the ratio of volume of nanomatrix to volume of T cells is greater than or equal to 1:25. In some embodiments, the ratio of volume of nanomatrix to volume of T cells is greater than or equal to 1:50. In some embodiments, the ratio of volume of nano matrix to volume of T cells is greater than or equal to 1:100. In some embodiments, the ratio of volume of nanomatrix to volume of T cells is greater than or equal to 1:200. In some embodiments, the ratio of volume of nanomatrix to volume of T cells is greater than or equal to 1:300. In some embodiments, the ratio of volume of nanomatrix to volume of T cells is greater than or equal to 1:400. In some embodiments, the ratio of volume of nanomatrix to volume of T cells is greater than or equal to 1:500. In some embodiments, the ratio of volume of nanomatrix to volume of T cells is greater than or equal to 1:600. In some embodiments, the ratio of volume of nanomatrix to volume of T cells is greater than or equal to 1:700. In some embodiments, the ratio of volume of nanomatrix to volume of T cells is greater than or equal to 1:800. In some embodiments, the ratio of volume of nanomatrix to volume of T cells is greater than or equal to 1:900. In some embodiments, the ratio of volume of nanomatrix to volume of T cells is greater than or equal to 1:1,000.

In some embodiments, the ratio of number of matrices to T cells is greater than or equal to 1:500. In some embodiments, the ratio of number of matrices to T cells is 1:500 to 1:750. In some embodiments, the ratio of number of matrices to T cells is 1:750 to 1:1,000. In some embodiments, the ratio of number of matrices to T cells is 1:1,000 to 1:1,250. In some embodiments, the ratio of number of matrices to T cells is 1:1,250 to 1:1,500. In some embodiments, the ratio of number of matrices to T cells is 1:1,500 to 1:1,750. In some embodiments, the ratio of number of matrices to T cells is 1:1,750 to 1:2,000. In some embodiments, the ratio of number of matrices to T cells is 1:2,000 to 1:2,250. In some embodiments, the ratio of number of matrices to T cells is 1:2,250 to 1:2,500. In some embodiments, the ratio of number of matrices to T cells is 1:2,500 to 1:2,750. In some embodiments, the ratio of number of matrices to T cells is 1:2,750 to 1:3,000. In some embodiments, the ratio of number of matrices to T cells is 1:3,000 to 1:3,500. In some embodiments, the ratio of number of matrices to T cells is 1:3,500 to 1:4,000. In some embodiments, the ratio of number of matrices to T cells is 1:4,000 to 1:5,000.

In some embodiments, the agonists are recombinant agonists. In some embodiments, the agonists are antibodies. In some embodiments, the antibodies are humanized antibodies. In some embodiments, the CD3 agonist is an OKT3 antibody or an UCHT1 antibody.

In another aspect of the method disclosed herein, the method for expanding a population of T cells comprises contacting the population of T cells with a nanomatrix comprising a colloidal suspension of matrices of polymer chains, wherein the matrices are attached to CD3 agonists and CD28 agonists, wherein the nanomatrix provides activation signals to the population of T cells, thereby activating and inducing the population of T cells to proliferate, wherein each matrix is 1 to 500 nm in length in its largest dimension, and wherein the method does not comprise the use of feeder cells during expansion of the population of T cells.

In some embodiments, the population of T cells contacted with the nanomatrix further comprises tumor cells. In some embodiments, the population of T cells is isolated from a subject and contacted with the nanomatrix without an additional expansion process of the population of T cells prior to contacting the population of T cells with the nanomatrix.

In some embodiments, the CD3 agonists and the CD28 agonists are attached to the same polymer chains. In some embodiments, the CD3 agonists and the CD28 agonists are attached to different polymer chains. In some embodiments, the CD3 agonists, or fragments thereof, are attached to the nanomatrix at 25 μg per mg of nanomatrix. In some embodiments, the CD3 agonists, or fragments thereof, are attached to the nanomatrix at about 5 μg to about 10 μg per mg of nanomatrix. In some embodiments, the CD3 agonists, or fragments thereof, are attached to the nanomatrix at about 10 μg to about 15 μg per mg of nanomatrix. In some embodiments, the CD3 agonists, or fragments thereof, are attached to the nanomatrix at about 15 μg to about 20 μg per mg of nanomatrix. In some embodiments, the CD3 agonists, or fragments thereof, are attached to the nanomatrix at about 20 μg to about 25 μg per mg of nanomatrix. In some embodiments, the CD3 agonists, or fragments thereof, are attached to the nanomatrix at about 25 μg to about 30 μg per mg of nanomatrix. In some embodiments, the CD3 agonists, or fragments thereof, are attached to the nanomatrix at about 30 μg to about 35 μg per mg of nanomatrix. In some embodiments, the CD3 agonists, or fragments thereof, are attached to the nanomatrix at about 35 μg to about g per mg of nanomatrix. In some embodiments, the CD3 agonists, or fragments thereof, are attached to the nanomatrix at about 40 μg to about 45 μg per mg of nanomatrix. In some embodiments, the CD3 agonists, or fragments thereof, are attached to the nanomatrix at about 45 μg to about 50 μg per mg of nanomatrix.

In some embodiments, the CD28 agonists, or fragments thereof, are attached to the nanomatrix at 25 μg per mg of nanomatrix. In some embodiments, the CD28 agonists, or fragments thereof, are attached to the nanomatrix at about 5 μg to about 10 μg per mg of nanomatrix. In some embodiments, the CD28 agonists, or fragments thereof, are attached to the nanomatrix at about 10 μg to about 15 μg per mg of nanomatrix. In some embodiments, the CD28 agonists, or fragments thereof, are attached to the nanomatrix at about 15 μg to about 20 μg per mg of nanomatrix. In some embodiments, the CD28 agonists, or fragments thereof, are attached to the nanomatrix at about 20 μg to about 25 μg per mg of nanomatrix. In some embodiments, the CD28 agonists, or fragments thereof, are attached to the nanomatrix at about 25 μg to about g per mg of nanomatrix. In some embodiments, the CD28 agonists, or fragments thereof, are attached to the nanomatrix at about 30 μg to about 35 μg per mg of nanomatrix. In some embodiments, the CD28 agonists, or fragments thereof, are attached to the nanomatrix at about 35 μg to about 40 μg per mg of nanomatrix. In some embodiments, the CD28 agonists, or fragments thereof, are attached to the nanomatrix at about 40 μg to about 45 μg per mg of nanomatrix. In some embodiments, the CD28 agonists, or fragments thereof, are attached to the nanomatrix at about 45 μg to about 50 μg per mg of nanomatrix. In some embodiments, the nanomatrix is TRANSACT™ a colloidal polymeric nanomatrix covalently attached to humanized recombinant agonists against human CD3 and CD28 from Miltenyi Biotec (MACS GMP T Cell Transact, Cat #130-019-011).

In some cases, the expansion phase(s) can be conducted in a supplemented cell culture medium comprising one or more T cell-stimulating cytokines IL-2, IL-7, IL-12, IL-15, IL-21, and combinations thereof and in some embodiments, an exogenous glutathione precursor. In some embodiments, the exogenous glutathione precursor is NAC. Media replacement is done (generally 30% to 99% media replacement via respiration with fresh media) until the cells are transferred to an alternative growth chamber. Alternative growth chambers include G-REX flasks and other gas permeable containers.

In some embodiments, a first expansion step is performed in a second culture medium. In some embodiments, the second culture medium comprises IL-7, IL-12 and/or IL-21. In some embodiments, the second culture medium comprises IL-7. In some embodiments, the second culture medium comprises IL-12. In some embodiments, the second culture medium comprises IL-21. In some embodiments, the second culture medium comprises IL-7 and IL-21. In some embodiments, the second culture medium comprises IL-7, IL-12 and II_-21, In some embodiments, one or more of the cytokines is provided in the second culture medium only at day 1, whereas other cytokines are replenished throughout the culture period. In some embodiments, one or more of IL-7, IL-12 and/or IL-21 are provided only on day 1 of culture. In some embodiments, IL-7 is provided only on day 1 of culture. In some embodiments, IL-12 is provided only on day 1 of culture. In some embodiments, IL-7 and IL-12 is provided only on day 1 of culture. In some embodiments, IL-21 is provided at regular intervals throughout the culture period. In some embodiments, IL-7 and IL-12 is provided only on day 1 of culture and IL-21 is provided at regular intervals throughout the culture period.

In some embodiments, a second expansion step is performed in a third culture medium after the first expansion step. In some embodiments, the T cells are cultured and/or expanded in the third culture medium after being cultured and/or expanded in the first and second culture media. In some embodiments, the third culture medium comprises one or more of IL-2, IL-12 and IL-21. In some embodiments, the third culture medium comprises IL-2. In some embodiments, the third culture medium comprises IL-12. In some embodiments, the third culture medium comprises IL-21. In some embodiments, the third culture medium comprises NAC. In some embodiments, the third culture medium comprises IL-12, IL-21 and NAC. In some embodiments, the third culture medium comprises IL-2, IL-12, IL-21 and NAC. In some embodiments, one or more of IL-2, IL-12 and/or IL-21 are provided only on day 1 of culture. In some embodiments, IL-12 is provided only on day 1 of culture. In some embodiments, IL-2 and IL-21 are provided at regular intervals throughout the culture period. In some embodiments, IL-12 is provided only on day 1 of culture and IL-2 and IL-21 are provided at regular intervals throughout the culture period.

In some cases, the expansion phase(s) may optionally be followed by a step wherein T cells are selected for transgene expression. Any selection method known in the art may be used, such as flow cytometry. Optionally, a cell viability assay can be performed after the expansion phase(s) using standard assays known in the art. For example, a trypan blue exclusion assay can be done on a sample of the bulk T cells, which selectively labels dead cells and allows a viability assessment. In some cases, T cell samples can be counted and viability determined using a Cellometer K2 automated cell counter (Nexcelom Bioscience, Lawrence, Mass.). In some embodiments, the T cells described herein are transposed with a mouse/human chimeric TCR. In some embodiments, at least one of the constant domains of the mouse/human chimeric TCR is a mouse sequence. In some embodiments, one or more of the variable domains of the mouse/human chimeric TCR is a human sequence. In some embodiments, binding agents that specifically bind mouse constant domains are used to select and/or enrich T cells expressing mouse/human chimeric TCRs. In some embodiments, the binding agents are antibodies. In some embodiments, the antibodies are monoclonal antibodies.

In some embodiments, the one or more T cell-stimulating cytokines utilized in the culture methods described herein is selected from the group consisting of IL-2, IL-7, IL-12, IL-15, IL-21, and combinations thereof. In some embodiments, the final concentration of the T cell-stimulating cytokine utilized in the first culture medium is about 10 U/ml to about 7,000 U/ml. In some embodiments, the final concentration of the T cell-stimulating cytokine utilized in the second culture medium is about 10 U/ml to about 7,000 U/ml. In some embodiments, the final concentration of the T cell-stimulating cytokine utilized in the third culture medium is about 10 U/ml to about 7,000 U/ml. In some embodiments, the final concentration of the T cell-stimulating cytokine utilized in the fourth medium is about 10 U/ml to about 7,000 U/ml.

In certain embodiments, the first culture medium utilized in the methods herein does not comprise IL-2, IL-12, or IL-21; both IL-2 and IL-21; both IL-2 and IL-12; both IL-12 and IL-21; or all of IL-2, IL-12 and IL-21. In certain embodiments, the second culture medium does not comprise IL-2, IL-15 or IL-21; both IL-2 and IL-21; both IL-2 and IL-15; both IL-15 and IL-21; or all of IL-2, IL-15 and IL-21. In certain embodiments, the third culture medium does not comprise IL-2, IL-7 or IL-15; both IL-2 and IL-15; both IL-2 and IL-7; both IL-7 or IL15; or all of IL-2, IL-7 and IL-15.

In some embodiments, the culture medium utilized in the expansion phase(s) is selected from the group consisting of the second culture medium and the third culture medium. In some embodiments, the medium utilized in the first expansion phase is the second culture medium. In some embodiments, a second expansion phase is performed and the culture medium utilized in the second expansion phase is the third culture medium.

In some embodiments, the culture medium utilized in the pre-expansion recovery step or one or more of the expansion phases is supplemented with one or more T cell-stimulating cytokine at a time interval selected from the group consisting of 1 day, 2 days, 3 days, 4 days, 5 days, and 6 days. In some embodiments, the medium is supplemented with the T cell-stimulating cytokine at a time interval ranging from 1-2 days, 2-3 days, 3-4 days, 4-5 days, or 5-6 days. In some embodiments, the time interval is 1 day. In some embodiments, the time interval is 2 days. In some embodiments, the time interval is 3 days. In some embodiments, the time interval is 4 days. In some embodiments, the time interval is 5 days. In some embodiments, the time interval is 6 days.

In some embodiments, the medium utilized in the pre-expansion recovery step one or more of the expansion phases is changed at a time interval selected from the group consisting of 1 day, 2 days, 3 days, 4 days, 5 days, and 6 days. In one embodiment, 30% to 99% of the medium utilized in one or more of the expansion phases is changed at a time interval selected from the group consisting of 1 day, 2 days, 3 days, 4 days, 5 days, and 6 days.

In some embodiments, the final concentration of the T cell-stimulating cytokine in any of the culture media described herein is 10 U/ml to 7,000 U/ml. In some embodiments, the final concentration of the T cell-stimulating cytokine is 100 U/ml to 200 U/ml. In some embodiments, the final concentration of the T cell-stimulating cytokine is 200 U/ml to 300 U/ml. In some embodiments, the final concentration of the T cell-stimulating cytokine is 300 U/ml to 400 U/ml. In some embodiments, the final concentration of the T cell-stimulating cytokine is 400 U/ml to 500 U/ml. In some embodiments, the final concentration of the T cell-stimulating cytokine is 500 U/ml to 600 U/ml. In some embodiments, the final concentration of the T cell-stimulating cytokine is 600 U/ml to 700 U/ml. In some embodiments, the final concentration of the T cell-stimulating cytokine is 700 U/ml to 800 U/ml. In some embodiments, the final concentration of the T cell-stimulating cytokine is 800 U/ml to 900 U/ml. In some embodiments, the final concentration of the T cell-stimulating cytokine is 900 U/ml to 1000 U/ml. In some embodiments, the final concentration of the T cell-stimulating cytokine is 1,000 U/ml to 1,500 U/ml. In some embodiments, the final concentration of the T cell-stimulating cytokine is 1,500 U/ml to 2,000 U/ml. In some embodiments, the final concentration of the T cell-stimulating cytokine is 2,000 U/ml to 2,500 U/ml. In some embodiments, the final concentration of the T cell-stimulating cytokine is 2,500 U/ml to 3,000 U/ml. In some embodiments, the final concentration of the T cell-stimulating cytokine is 3,000 U/ml to 3,500 U/ml. In some embodiments, the final concentration of the T cell-stimulating cytokine is 3,500 U/ml to 4,000 U/ml. In some embodiments, the final concentration of the T cell-stimulating cytokine is 4,000 U/ml to 4,500 U/ml. In some embodiments, the final concentration of the T cell-stimulating cytokine is 4,500 U/ml to 5,000 U/ml. In some embodiments, the final concentration of the T cell-stimulating cytokine is 5,000 U/ml to 5,500 U/ml. In some embodiments, the final concentration of the T cell-stimulating cytokine is 5,500 U/ml to 6,000 U/ml. In some embodiments, the final concentration of the T cell-stimulating cytokine is 6,000 U/ml to 6,500 U/ml. In some embodiments, the final concentration of the T cell-stimulating cytokine is 6,500 U/ml to 7,000 U/ml.

In some embodiments, the final concentration of the T cell-stimulating cytokine is 1-10,000 ng/ml. In some embodiments, the final concentration of T cell-stimulating cytokine utilized is less than 10,000 ng/ml, optionally less than 1000, 750, 500, 400, 300, 200, 100, 50, or 30 ng/ml. In some embodiments, the final concentration of T cell-stimulating cytokine utilized is from about 5 ng/ml to about 30 ng/ml. In further embodiments, the final concentration of T cell-stimulating cytokine utilized is greater than 5 ng/ml.

In some embodiments, the final concentration of the one or more T cell-stimulating cytokines in the first culture medium is greater than 1 ng/ml. In further embodiments, the final concentration of the one or more T cell-stimulating cytokines in the first culture medium is greater than 1 ng/ml to about 100 ng/ml. In a specific embodiment, the final concentration of the one or more T cell-stimulating cytokines in the first culture medium is from about 5 ng/ml to about 30 ng/ml.

In some embodiments, the final concentration of the one or more T cell-stimulating cytokines in the second culture medium is greater than 1 ng/ml. In further embodiments, the final concentration of the one or more T cell-stimulating cytokines in the second culture medium is greater than 1 ng/ml to about 100 ng/ml. In a specific embodiment, the final concentration of the one or more T cell-stimulating cytokines in the second culture medium is from about 5 ng/ml to about 30 ng/ml.

In some embodiments, the final concentration of the one or more T cell-stimulating cytokines in the third culture medium is greater than 1 ng/ml. In further embodiments, the final concentration of the one or more T cell-stimulating cytokines in the third culture medium is greater than 1 ng/ml to about 100 ng/ml. In a specific embodiment, the final concentration of the one or more T cell-stimulating cytokines in the third culture medium is from about 5 ng/ml to about 30 ng/ml.

The one or more T-cell stimulating cytokines can be any cytokine effective in stimulating T-cells. In some embodiments, the T cell-stimulating cytokine is IL-2, IL-7, IL-12, IL-15 and/or IL-21.

In some embodiments, the T cells are expanded for up to a total of 3, 4, 5, 6, 7, 8, 9, 10, 11, 12, 13, 14, 15, 16, 17, 18, 19, 20, 21, 22, 23, 24, 25, 26, 27 or 28 days from electroporation. In some embodiments, the T cells are expanded for a total of 9-25 days, 9-21 days, or 9-14 days. In some embodiments, the T cells are expanded for up to a total of 9 days. In some embodiments, the T cells are expanded for up to a total of 10 days. In some embodiments, the T cells are expanded for up to a total of 11 days. In some embodiments, the T cells are expanded for up to a total of 12 days. In some embodiments, the T cells are expanded for up to a total of 13 days. In some embodiments, the T cells are expanded for up to a total of 14 days. In some embodiments, the T cells are expanded for up to a total of 15 days. In some embodiments, the T cells are expanded for up to a total of 16 days. In some embodiments, the T cells are expanded for up to a total of 17 days. In some embodiments, the T cells are expanded for up to a total of 18 days. In some embodiments, the T cells are expanded for up to a total of 19 days. In some embodiments, the T cells are expanded for up to a total of 20 days. In some embodiments, the T cells are expanded for up to a total of 21 days. In some embodiments, the T cells are expanded for up to a total of 22 days. In some embodiments, the T cells are expanded for up to a total of 23 days. In some embodiments, the T cells are expanded for up to a total of 24 days. In some embodiments, the T cells are expanded for up to a total of 25 days. In some embodiments, the T cells are expanded for up to a total of 26 days. In some embodiments, the T cells are expanded for up to a total of 27 days. In some embodiments, the T cells are expanded for up to a total of 28 days.

In some embodiments, the population of T cells is expanded 50 to 1,000,000-fold. In some embodiments, the population of T cells is expanded 50 to 500-fold. In some embodiments, the population of T cells is expanded 500 to 1,000-fold. In some embodiments, the population of T cells is expanded 500 to 4,000-fold. In some embodiments, the population of T cells is expanded 1,000 to 2,500-fold. In some embodiments, the population of T cells is expanded 2,500 to 5,000-fold. In some embodiments, the population of T cells is expanded 5,000 to 10,000-fold. In some embodiments, the population of T cells is expanded 10,000 to 20,000-fold. In some embodiments, the population of T cells is expanded 20,000 to 30,000-fold. In some embodiments, the population of T cells is expanded 30,000 to 40,000-fold. In some embodiments, the population of T cells is expanded 40,000 to 50,000-fold. In some embodiments, the population of T cells is expanded 50,000 to 100,000-fold. In some embodiments, the population of T cells is expanded 100,000 to 150,000-fold. In some embodiments, the population of T cells is expanded 150,000 to 200,000-fold. In some embodiments, the population of T cells is expanded 200,000 to 250,000-fold. In some embodiments, the population of T cells is expanded 250,000 to 300,000-fold. In some embodiments, the population of T cells is expanded 300,000 to 350,000-fold. In some embodiments, the population of T cells is expanded 350,000 to 400,000-fold. In some embodiments, the population of T cells is expanded 400,000 to 450,000-fold. In some embodiments, the population of T cells is expanded 450,000 to 500,000-fold. In some embodiments, the population of T cells is expanded 500,000 to 550,000-fold. In some embodiments, the population of T cells is expanded 550,000 to 600,000-fold. In some embodiments, the population of T cells is expanded 600,000 to 650,000-fold. In some embodiments, the population of T cells is expanded 650,000 to 700,000-fold. In some embodiments, the population of T cells is expanded 700,000 to 750,000-fold. In some embodiments, the population of T cells is expanded 750,000 to 800,000-fold. In some embodiments, the population of T cells is expanded 800,000 to 850,000-fold. In some embodiments, the population of T cells is expanded 850,000 to 900,000-fold. In some embodiments, the population of T cells is expanded 900,000 to 950,000-fold. In some embodiments, the population of T cells is expanded 950,000 to 1,000,000-fold.

In some embodiments, the population of T cells is expanded from an initial population of T cells of between 10,000 and 1×10⁹ T cells. In some embodiments, the population of T cells is expanded from an initial population of T cells of between 10,000 and 100,000 T cells. In some embodiments, the population of T cells is expanded from an initial population of T cells of between 100,000 and 250,000 T cells. In some embodiments, the population of T cells is expanded from an initial population of T cells of between 250,000 and 500,000 T cells. In some embodiments, the population of T cells is expanded from an initial population of T cells of between 500,000 and 750,000 T cells. In some embodiments, the population of T cells is expanded from an initial population of T cells of between 750,000 and 1,000,000 T cells. In some embodiments, the population of T cells is expanded from an initial population of T cells of between 1×10⁶ and 2×10⁶ T cells. In some embodiments, the population of T cells is expanded from an initial population of T cells of between 2×10⁶ and 3×10⁶ T cells. In some embodiments, the population of T cells is expanded from an initial population of T cells of between 3×10⁶ and 4×10⁶ T cells. In some embodiments, the population of T cells is expanded from an initial population of T cells of between 4×10⁶ and 5×10⁶ T cells. In some embodiments, the population of T cells is expanded from an initial population of T cells of between 5×10⁶ and 6×10⁶ T cells. In some embodiments, the population of T cells is expanded from an initial population of T cells of between 6×10⁶ and 7×10⁶ T cells. In some embodiments, the population of T cells is expanded from an initial population of T cells of between 7×10⁶ and 8×10⁶ T cells. In some embodiments, the population of T cells is expanded from an initial population of T cells of between 8×10⁶ and 9×10⁶ T cells. In some embodiments, the population of T cells is expanded from an initial population of T cells of between 9×10⁶ and 1×10⁷ T cells. In some embodiments, the population of T cells is expanded from an initial population of T cells of between 1×10⁷ and 5×10⁷ T cells. In some embodiments, the population of T cells is expanded from an initial population of T cells of between 5×10⁷ and 1×10⁸ T cells. In some embodiments, the population of T cells is expanded from an initial population of T cells of between 1×10⁸ and 5×10⁸ T cells. In some embodiments, the population of T cells is expanded from an initial population of T cells of between 5×10⁸ and 1×10⁹ T cells.

In some embodiments, the population of T cells is expanded at least 50-fold at day 14 of the expansion. In some embodiments, the population of T cells is expanded at least 100-fold at day 14 of the expansion. In some embodiments, the population of T cells is expanded at least 500-fold at day 14 of the expansion. In some embodiments, the population of T cells is expanded at least 1000-fold at day 14 of the expansion. In some embodiments, the population of T cells is expanded at least 1,500-fold at day 14 of the expansion. In some embodiments, the population of T cells is expanded at least 5,000-fold at day 14 of the expansion. In some embodiments, the population of T cells is expanded at least 7,500-fold at day 14 of the expansion. In some embodiments, the population of T cells is expanded at least 10,000-fold at day 14 of the expansion. In some embodiments, the population of T cells is expanded at least 15,000-fold at day 14 of the expansion. In some embodiments, the population of T cells is expanded at least 20,000-fold at day 14 of the expansion. In some embodiments, the population of T cells is expanded at least 25,000-fold at day 14 of the expansion. In some embodiments, the population of T cells is expanded at least 30,000-fold at day 14 of the expansion. In some embodiments, the population of T cells is expanded at least 40,000-fold at day 14 of the expansion. In some embodiments, the population of T cells is expanded at least 50,000-fold at day 14 of the expansion. In some embodiments, the population of T cells is expanded at least 60,000-fold at day 14 of the expansion. In some embodiments, the population of T cells is expanded at least 70,000-fold at day 14 of the expansion. In some embodiments, the population of T cells is expanded at least 80,000-fold at day 14 of the expansion. In some embodiments, the population of T cells is expanded at least 90,000-fold at day 14 of the expansion. In some embodiments, the population of T cells is expanded at least 100,000-fold at day 14 of the expansion. In some embodiments, the population of T cells is expanded at least 110,000-fold at day 14 of the expansion. In some embodiments, the population of T cells is expanded at least 120,000-fold at day 14 of the expansion. In some embodiments, the population of T cells is expanded at least 130,000-fold at day 14 of the expansion. In some embodiments, the population of T cells is expanded at least 140,000-fold at day 14 of the expansion. In some embodiments, the population of T cells is expanded at from 1,000-fold to 5,000-fold at day 14 of the expansion.

In some embodiments, the population of T cells is expanded at least 10-fold at day 10 of the expansion. In some embodiments, the population of T cells is expanded at least 50-fold at day 10 of the expansion. In some embodiments, the population of T cells is expanded at least 100-fold at day 10 of the expansion. In some embodiments, the population of T cells is expanded at least 150-fold at day 10 of the expansion. In some embodiments, the population of T cells is expanded at least 500-fold at day 10 of the expansion. In some embodiments, the population of T cells is expanded at least 750-fold at day 10 of the expansion. In some embodiments, the population of T cells is expanded at least 1000-fold at day 10 of the expansion. In some embodiments, the population of T cells is expanded at least 1500-fold at day 10 of the expansion. In some embodiments, the population of T cells is expanded at least 2000-fold at day 10 of the expansion. In some embodiments, the population of T cells is expanded at least 2500-fold at day 10 of the expansion. In some embodiments, the population of T cells is expanded at least 3000-fold at day 10 of the expansion. In some embodiments, the population of T cells is expanded at least 4000-fold at day 10 of the expansion. In some embodiments, the population of T cells is expanded at least 5000-fold at day 10 of the expansion. In some embodiments, the population of T cells is expanded at least 6000-fold at day 10 of the expansion. In some embodiments, the population of T cells is expanded at least 7000-fold at day 10 of the expansion. In some embodiments, the population of T cells is expanded at least 8000-fold at day 10 of the expansion. In some embodiments, the population of T cells is expanded at least 9000-fold at day 10 of the expansion. In some embodiments, the population of T cells is expanded at least 10,000-fold at day 10 of the expansion.

In some embodiments, the population of T cells is expanded at most 150,000-fold at day 14 of the expansion. In some embodiments, the population of T cells is expanded at most 5,000-fold at day 14 of the expansion. In some embodiments, the population of T cells is expanded at most 7,500-fold at day 14 of the expansion. In some embodiments, the population of T cells is expanded at most 10,000-fold at day 14 of the expansion. In some embodiments, the population of T cells is expanded at most 15,000-fold at day 14 of the expansion. In some embodiments, the population of T cells is expanded at most 20,000-fold at day 14 of the expansion. In some embodiments, the population of T cells is expanded at most 25,000-fold at day 14 of the expansion. In some embodiments, the population of T cells is expanded at most 30,000-fold at day 14 of the expansion. In some embodiments, the population of T cells is expanded at most 40,000-fold at day 14 of the expansion. In some embodiments, the population of T cells is expanded at most 50,000-fold at day 14 of the expansion. In some embodiments, the population of T cells is expanded at most 60,000-fold at day 14 of the expansion. In some embodiments, the population of T cells is expanded at most 70,000-fold at day 14 of the expansion. In some embodiments, the population of T cells is expanded at most 80,000-fold at day 14 of the expansion. In some embodiments, the population of T cells is expanded at most 90,000-fold at day 14 of the expansion. In some embodiments, the population of T cells is expanded at most 100,000-fold at day 14 of the expansion. In some embodiments, the population of T cells is expanded at most 110,000-fold at day 14 of the expansion. In some embodiments, the population of T cells is expanded at most 120,000-fold at day 14 of the expansion. In some embodiments, the population of T cells is expanded at most 130,000-fold at day 14 of the expansion. In some embodiments, the population of T cells is expanded at most 140,000-fold at day 14 of the expansion.

In some embodiments, the population of T cells is expanded at least 100-fold at day 21 of the expansion. In some embodiments, the population of T cells is expanded at least 500-fold at day 21 of the expansion. In some embodiments, the population of T cells is expanded at least 1,000-fold at day 21 of the expansion. In some embodiments, the population of T cells is expanded at least 5,000-fold at day 21 of the expansion. In some embodiments, the population of T cells is expanded at least 10,000-fold at day 21 of the expansion. In some embodiments, the population of T cells is expanded at least 15,000-fold at day 21 of the expansion. In some embodiments, the population of T cells is expanded at least 20,000-fold at day 21 of the expansion. In some embodiments, the population of T cells is expanded at least 25,000-fold at day 21 of the expansion. In some embodiments, the population of T cells is expanded at least 30,000-fold at day 21 of the expansion. In some embodiments, the population of T cells is expanded at least 40,000-fold at day 21 of the expansion. In some embodiments, the population of T cells is expanded at least 50,000-fold at day 21 of the expansion. In some embodiments, the population of T cells is expanded at least 60,000-fold at day 21 of the expansion. In some embodiments, the population of T cells is expanded at least 70,000-fold at day 21 of the expansion. In some embodiments, the population of T cells is expanded at least 80,000-fold at day 21 of the expansion. In some embodiments, the population of T cells is expanded at least 90,000-fold at day 21 of the expansion. In some embodiments, the population of T cells is expanded at least 100,000-fold at day 21 of the expansion. In some embodiments, the population of T cells is expanded at least 110,000-fold at day 21 of the expansion. In some embodiments, the population of T cells is expanded at least 120,000-fold at day 21 of the expansion. In some embodiments, the population of T cells is expanded at least 130,000-fold at day 21 of the expansion. In some embodiments, the population of T cells is expanded at least 140,000-fold at day 21 of the expansion. In some embodiments, the population of T cells is expanded at least 150,000-fold at day 21 of the expansion. In some embodiments, the population of T cells is expanded at least 200,000-fold at day 21 of the expansion. In some embodiments, the population of T cells is expanded at least 300,000-fold at day 21 of the expansion. In some embodiments, the population of T cells is expanded at least 400,000-fold at day 21 of the expansion.

In some embodiments, the population of T cells is expanded at most 500,000-fold at day 21 of the expansion. In some embodiments, the population of T cells is expanded at most 20,000-fold at day 21 of the expansion. In some embodiments, the population of T cells is expanded at most 25,000-fold at day 21 of the expansion. In some embodiments, the population of T cells is expanded at most 30,000-fold at day 21 of the expansion. In some embodiments, the population of T cells is expanded at most 40,000-fold at day 21 of the expansion. In some embodiments, the population of T cells is expanded at most 50,000-fold at day 21 of the expansion. In some embodiments, the population of T cells is expanded at most 60,000-fold at day 21 of the expansion. In some embodiments, the population of T cells is expanded at most 70,000-fold at day 21 of the expansion. In some embodiments, the population of T cells is expanded at most 80,000-fold at day 21 of the expansion. In some embodiments, the population of T cells is expanded at most 90,000-fold at day 21 of the expansion. In some embodiments, the population of T cells is expanded at most 100,000-fold at day 21 of the expansion. In some embodiments, the population of T cells is expanded at most 110,000-fold at day 21 of the expansion. In some embodiments, the population of T cells is expanded at most 120,000-fold at day 21 of the expansion. In some embodiments, the population of T cells is expanded at most 130,000-fold at day 21 of the expansion. In some embodiments, the population of T cells is expanded at most 140,000-fold at day 21 of the expansion. In some embodiments, the population of T cells is expanded at most 150,000-fold at day 21 of the expansion. In some embodiments, the population of T cells is expanded at most 200,000-fold at day 21 of the expansion. In some embodiments, the population of T cells is expanded at most 300,000-fold at day 21 of the expansion. In some embodiments, the population of T cells is expanded at most 400,000-fold at day 21 of the expansion.

In some embodiments, the population of T cells is expanded to produce an expanded population of T cells, wherein at least 2% of the expanded population expresses a recombinant protein encoded by the nucleic acid introduced into the T cells by electroporation. In some embodiments, the population of T cells is expanded to produce an expanded population of T cells, wherein at least 3% of the expanded population expresses a recombinant protein encoded by the nucleic acid introduced into the T cells by electroporation. In some embodiments, the population of T cells is expanded to produce an expanded population of T cells, wherein at least 4% of the expanded population expresses a recombinant protein encoded by the nucleic acid introduced into the T cells by electroporation. In some embodiments, the population of T cells is expanded to produce an expanded population of T cells, wherein at least 5% of the expanded population expresses a recombinant protein encoded by the nucleic acid introduced into the T cells by electroporation. In some embodiments, the population of T cells is expanded to produce an expanded population of T cells, wherein at least 6% of the expanded population expresses a recombinant protein encoded by the nucleic acid introduced into the T cells by electroporation. In some embodiments, the population of T cells is expanded to produce an expanded population of T cells, wherein at least 7% of the expanded population expresses a recombinant protein encoded by the nucleic acid introduced into the T cells by electroporation. In some embodiments, the population of T cells is expanded to produce an expanded population of T cells, wherein at least 8% of the expanded population expresses a recombinant protein encoded by the nucleic acid introduced into the T cells by electroporation. In some embodiments, the population of T cells is expanded to produce an expanded population of T cells, wherein at least 9% of the expanded population expresses a recombinant protein encoded by the nucleic acid introduced into the T cells by electroporation. In some embodiments, the population of T cells is expanded to produce an expanded population of T cells, wherein at least 10% of the expanded population expresses a recombinant protein encoded by the nucleic acid introduced into the T cells by electroporation. In some embodiments, the population of T cells is expanded to produce an expanded population of T cells, wherein at least 15% of the expanded population expresses a recombinant protein encoded by the nucleic acid introduced into the T cells by electroporation. In some embodiments, the population of T cells is expanded to produce an expanded population of T cells, wherein at least 20% of the expanded population expresses a recombinant protein encoded by the nucleic acid introduced into the T cells by electroporation. In some embodiments, the population of T cells is expanded to produce an expanded population of T cells, wherein at least 25% of the expanded population expresses a recombinant protein encoded by the nucleic acid introduced into the T cells by electroporation. In some embodiments, the population of T cells is expanded to produce an expanded population of T cells, wherein at least 30% of the expanded population expresses a recombinant protein encoded by the nucleic acid introduced into the T cells by electroporation. In some embodiments, the population of T cells is expanded to produce an expanded population of T cells, wherein at least 35% of the expanded population expresses a recombinant protein encoded by the nucleic acid introduced into the T cells by electroporation. In some embodiments, the population of T cells is expanded to produce an expanded population of T cells, wherein at least 40% of the expanded population expresses a recombinant protein encoded by the nucleic acid introduced into the T cells by electroporation. In some embodiments, the recombinant protein is a TCR, an IL-15 (e.g., mbIL15), or both. In some embodiments, the recombinant protein is detected in the expanded T cells by flow cytometry. In some embodiments, the recombinant protein in detected in the expanded T cells prior to an enrichment step.

In another aspect, the present disclosure provides a population of engineered T cells, wherein most of the engineered T cells in the population comprise an exogenous TCR or functional fragment thereof, and wherein more than 2%. 3%. 4%, 5%, 6%, 7%, 8%, 9%, 10%, 15%, 20%, 25%, 30%, 35% or 40% of the population of engineered T cells are CCR7+/CD45RO+. In another aspect, the present disclosure provides a population of engineered T cells, wherein most of the engineered T cells in the population comprise an exogenous TCR or functional fragment thereof, and wherein more than 2%, 3%, 4%, 5%, 6%, 7%, 8%, 9%, 10%, 15%, 20%, 25%, 30%, 35% or 40% of the population of engineered T cells are memory T cells (e.g., a central memory T cell, an effector memory T cell, a stem cell-like memory T cells). In some embodiments, the T cells are electroporated with a vector that expresses the exogenous TCR or functional fragment thereof. In some embodiments, the T cells are cultured and/or expanded according to any of the methods provided herein.

In another aspect, the present disclosure provides a population of engineered T cells, wherein most of the engineered T cells in the population comprise an exogenous TCR or functional fragment thereof, and wherein more than 2%, 3%, 4%, 5%, 6%, 7%, 8%, 9%, 10%, 15%, 20%, 25%, 30%, 35% or 40% of the population of engineered T cells are CD95+/CD62L+. In another aspect, the present disclosure provides a population of engineered T cells, wherein most of the engineered T cells in the population comprise an exogenous TCR or functional fragment thereof, and wherein more than 2%, 3%, 4%, 5%, 6%, 7%, 8%, 9%, 10%, 15%, 20%, 25%, 30%, 35% or 40% of the population of engineered T cells are memory T cells (e.g., a central memory T cell, an effector memory T cell, a stem cell memory T cells). In some embodiments, the T cells are electroporated with a vector that expresses the exogenous TCR or functional fragment thereof. In some embodiments, the T cells are cultured and/or expanded according to any of the methods provided herein.

a. T-Cell Stimulating Cytokines

The T-cell stimulating cytokine can be any cytokine effective in stimulating T-cells. In some embodiments, the T cell-stimulating cytokine is IL-2, IL-7, IL-12, IL-15 and/or IL-21.

In some embodiments, the methods disclosed herein comprise contacting the electroporated T cells with the cytokine IL-15. In some embodiments, the T cells are contacted with the cytokine IL-15 every other day. In some embodiments, the T cells are contacted with the cytokine IL-15 in time intervals of 2, 3, 4, 5, or 6 days. In some embodiments, the T cells are contacted with the cytokine IL-15 in a time interval of 2 days. In some embodiments, the T cells are contacted with the cytokine IL-15 in a time interval of 3 days. In some embodiments, the T cells are contacted with the cytokine IL-15 in a time interval of 4 days. In some embodiments, the T cells are contacted with the cytokine IL-15 in a time interval of 5 days. In some embodiments, the T cells are contacted with the cytokine IL-15 in a time interval of 6 days.

Concentrations of T-cell stimulating cytokines are expressed either as ng/ml or U (“units”)/ml, herein. The terms International Units (IU) and units are used interchangeably, herein. Conversion of units between ng/ml and U/ml can vary based on the cytokine used or even the source of a given cytokine. In some embodiments, 2 U/ml of T-cell stimulating cytokine would be the equivalent of 1 ng/ml of T-cell stimulating cytokine. Thus, 20 U/ml of T-cell stimulating cytokine would be the equivalent of 10 ng/ml of T-cell stimulating cytokine, etc. In some embodiments, about 2 U/ml of T-cell stimulating cytokine would be the equivalent of about 1 ng/ml of T-cell stimulating cytokine. As provided above, in some embodiments, the T cell-stimulating cytokine is IL-2, IL-7, IL-12, IL-15 and/or IL-21. In some embodiments, the conversion provided herein can vary by up to 20% more or less. For example, in some embodiments, 1 unit/ml is the equivalent of 1.6 mg/ml-2.4 mg/ml. In some embodiments, the conversion provided herein can vary by up to 10% more or less. For example, in some embodiments, 1 unit/ml is the equivalent of 1.8 mg/mi-2.2 mg/ml.

In some embodiments, the final concentration of the cytokine IL-15 in the cell culture media is 0.5 ng/ml to 10,000 ng/ml. In some embodiments, the final concentration of the cytokine IL-15 in the cell culture media is 10 ng/ml to 10,000 ng/ml. In some embodiments, the final concentration of the cytokine IL-15 in the cell culture media is 0.5 ng/ml to 10 ng/ml. In some embodiments, the final concentration of the cytokine IL-15 in the cell culture media is 10 ng/ml to 25 ng/ml. In some embodiments, the final concentration of the cytokine IL-15 in the cell culture media is 25 ng/ml to 50 ng/ml. In some embodiments, the final concentration of the cytokine IL-15 in the cell culture media is 50 ng/ml to 75 ng/ml. In some embodiments, the final concentration of the cytokine IL-15 in the cell culture media is 75 ng/ml to 100 ng/ml. In some embodiments, the final concentration of the cytokine IL-15 in the cell culture media is 100 ng/ml to 200 ng/ml. In some embodiments, the final concentration of the cytokine IL-15 in the cell culture media is 200 ng/ml to 300 ng/ml. In some embodiments, the final concentration of the cytokine IL-15 in the cell culture media is 300 ng/ml to 400 ng/ml. In some embodiments, the final concentration of the cytokine IL-15 in the cell culture media is 400 ng/ml to 500 ng/ml. In some embodiments, the final concentration of the cytokine IL-15 in the cell culture media is 500 ng/ml to 600 ng/ml. In some embodiments, the final concentration of the cytokine IL-15 in the cell culture media is 600 ng/ml to 700 ng/ml. In some embodiments, the final concentration of the cytokine IL-15 in the cell culture media is 700 ng/ml to 800 ng/ml. In some embodiments, the final concentration of the cytokine IL-15 in the cell culture media is 800 ng/ml to 900 ng/ml. In some embodiments, the final concentration of the cytokine IL-15 in the cell culture media is 900 ng/ml to 1000 ng/ml. In some embodiments, the final concentration of the cytokine IL-15 in the cell culture media is 1,000 ng/ml to 1,500 ng/ml. In some embodiments, the final concentration of the cytokine IL-15 in the cell culture media is 1,500 ng/ml to 2,000 ng/ml. In some embodiments, the final concentration of the cytokine IL-15 in the cell culture media is 2,000 ng/ml to 2,500 ng/ml. In some embodiments, the final concentration of the cytokine IL-15 in the cell culture media is 2,500 ng/ml to 3,000 ng/ml. In some embodiments, the final concentration of the cytokine IL-15 in the cell culture media is 3,000 ng/ml to 3,500 ng/ml. In some embodiments, the final concentration of the cytokine IL-15 in the cell culture media is 3,500 ng/ml to 4,000 ng/ml. In some embodiments, the final concentration of the cytokine IL-15 in the cell culture media is 4,000 ng/ml to 4,500 ng/ml. In some embodiments, the final concentration of the cytokine IL-15 in the cell culture media is 4,500 ng/ml to 5,000 ng/ml. In some embodiments, the final concentration of the cytokine IL-15 in the cell culture media is 5,000 ng/ml to 5,500 ng/ml. In some embodiments, the final concentration of the cytokine IL-15 in the cell culture media is 5,500 ng/ml to 6,000 ng/ml. In some embodiments, the final concentration of the cytokine IL-15 in the cell culture media is 6,000 ng/ml to 6,500 ng/ml. In some embodiments, the final concentration of the cytokine IL-15 in the cell culture media is 6,500 ng/ml to 7,000 ng/ml. In some embodiments, the final concentration of the cytokine IL-15 in the cell culture media is 7,000 ng/ml to 7,500 ng/ml. In some embodiments, the final concentration of the cytokine IL-15 in the cell culture media is 7,500 ng/ml to 8,000 ng/ml. In some embodiments, the final concentration of the cytokine IL-15 in the cell culture media is 8,000 ng/ml to 8,500 ng/ml. In some embodiments, the final concentration of the cytokine IL-15 in the cell culture media is 8,500 ng/ml to 9,000 ng/ml. In some embodiments, the final concentration of the cytokine IL-15 in the cell culture media is 9,000 ng/ml to 9,500 ng/ml. In some embodiments, the final concentration of the cytokine IL-15 in the cell culture media is 9,500 ng/ml to 10,000 ng/ml.

In some embodiments, the final concentration of the cytokine IL-15 in the cell culture media is 1 U/ml to 20,000 U/ml. In some embodiments, the final concentration of the cytokine IL-15 in the cell culture media is 2 U/ml to 20,000 U/ml. In some embodiments, the final concentration of the cytokine IL-15 in the cell culture media is 20 U/ml to 20,000 U/ml. In some embodiments, the final concentration of the cytokine IL-15 in the cell culture media is 2 U/ml to 20 U/ml. In some embodiments, the final concentration of the cytokine IL-15 in the cell culture media is 20 U/ml to 50 U/ml. In some embodiments, the final concentration of the cytokine IL-15 in the cell culture media is 50 U/ml to 100 U/ml. In some embodiments, the final concentration of the cytokine IL-15 in the cell culture media is 100 U/ml to 150 U/ml. In some embodiments, the final concentration of the cytokine IL-15 in the cell culture media is 150 U/ml to 200 U/ml. In some embodiments, the final concentration of the cytokine IL-15 in the cell culture media is 200 U/ml to 400 U/ml. In some embodiments, the final concentration of the cytokine IL-15 in the cell culture media is 400 U/ml to 600 U/ml. In some embodiments, the final concentration of the cytokine IL-15 in the cell culture media is 600 U/ml to 800 U/ml. In some embodiments, the final concentration of the cytokine IL-15 in the cell culture media is 800 U/ml to 1000 U/ml. In some embodiments, the final concentration of the cytokine IL-15 in the cell culture media is 1000 U/ml to 1200 U/ml. In some embodiments, the final concentration of the cytokine IL-15 in the cell culture media is 1200 U/ml to 1400 U/ml. In some embodiments, the final concentration of the cytokine IL-15 in the cell culture media is 1400 U/ml to 1600 U/ml. In some embodiments, the final concentration of the cytokine IL-15 in the cell culture media is 1600 U/ml to 1800 U/ml. In some embodiments, the final concentration of the cytokine IL-15 in the cell culture media is 1800 U/ml to 2000 U/ml. In some embodiments, the final concentration of the cytokine IL-15 in the cell culture media is 2000 U/ml to 3000 U/ml. In some embodiments, the final concentration of the cytokine IL-15 in the cell culture media is 3000 U/ml to 4000 U/ml. In some embodiments, the final concentration of the cytokine IL-15 in the cell culture media is 4000 U/ml to 5000 U/ml. In some embodiments, the final concentration of the cytokine IL-15 in the cell culture media is 5000 U/ml to 6000 U/ml. In some embodiments, the final concentration of the cytokine IL-15 in the cell culture media is 6000 U/ml to 7000 U/ml. In some embodiments, the final concentration of the cytokine IL-15 in the cell culture media is 7000 U/ml to 8000 U/ml. In some embodiments, the final concentration of the cytokine IL-15 in the cell culture media is 8000 U/ml to 9000 U/ml. In some embodiments, the final concentration of the cytokine IL-15 in the cell culture media is 9000 U/ml to 10,000 U/ml. In some embodiments, the final concentration of the cytokine IL-15 in the cell culture media is 10,000 U/ml to 11,000 U/ml. In some embodiments, the final concentration of the cytokine IL-15 in the cell culture media is 11,000 U/ml to 12,000 U/ml. In some embodiments, the final concentration of the cytokine IL-15 in the cell culture media is 12,000 U/ml to 13,000 U/ml. In some embodiments, the final concentration of the cytokine IL-15 in the cell culture media is 13,000 U/ml to 14,000 U/ml. In some embodiments, the final concentration of the cytokine IL-15 in the cell culture media is 14,000 U/ml to 15,000 U/ml. In some embodiments, the final concentration of the cytokine IL-15 in the cell culture media is 15,000 U/ml to 16,000 U/ml. In some embodiments, the final concentration of the cytokine IL-15 in the cell culture media is 16,000 U/ml to 17,000 U/ml. In some embodiments, the final concentration of the cytokine IL-15 in the cell culture media is 17,000 U/ml to 18,000 U/ml. In some embodiments, the final concentration of the cytokine IL-15 in the cell culture media is 18,000 U/ml to 19,000 U/ml. In some embodiments, the final concentration of the cytokine IL-15 in the cell culture media is 19,000 U/ml to 20,000 U/ml.

In some embodiments, the methods disclosed herein comprise contacting the electroporated T cells with the cytokine IL-7. In some embodiments, the T cells are contacted with the cytokine IL-7 every other day. In some embodiments, the T cells are contacted with the cytokine IL-7 in time intervals of 2, 3, 4, 5, or 6 days. In some embodiments, the T cells are contacted with the cytokine IL-7 in a time interval of 2 days. In some embodiments, the T cells are contacted with the cytokine IL-7 in a time interval of 3 days. In some embodiments, the T cells are contacted with the cytokine IL-7 in a time interval of 4 days. In some embodiments, the T cells are contacted with the cytokine IL-7 in a time interval of 5 days. In some embodiments, the T cells are contacted with the cytokine IL-7 in a time interval of 6 days.

In some embodiments, the final concentration of the cytokine IL-7 in the cell culture media is 0.5 ng/ml to 10,000 ng/ml. In some embodiments, the final concentration of the cytokine IL-7 in the cell culture media is 10 ng/ml to 10,000 ng/ml. In some embodiments, the final concentration of the cytokine IL-7 in the cell culture media is 0.5 ng/ml to 10 ng/ml. In some embodiments, the final concentration of the cytokine IL-7 in the cell culture media is 10 ng/ml to 25 ng/ml. In some embodiments, the final concentration of the cytokine IL-7 in the cell culture media is 25 ng/ml to 50 ng/ml. In some embodiments, the final concentration of the cytokine IL-7 in the cell culture media is 50 ng/ml to 75 ng/ml. In some embodiments, the final concentration of the cytokine IL-7 in the cell culture media is 75 ng/ml to 100 ng/ml. In some embodiments, the final concentration of the cytokine IL-7 in the cell culture media is 100 ng/ml to 200 ng/ml. In some embodiments, the final concentration of the cytokine IL-7 in the cell culture media is 200 ng/ml to 300 ng/ml. In some embodiments, the final concentration of the cytokine IL-7 in the cell culture media is 300 ng/ml to 400 ng/ml. In some embodiments, the final concentration of the cytokine IL-7 in the cell culture media is 400 ng/ml to 500 ng/ml. In some embodiments, the final concentration of the cytokine IL-7 in the cell culture media is 500 ng/ml to 600 ng/ml. In some embodiments, the final concentration of the cytokine IL-7 in the cell culture media is 600 ng/ml to 700 ng/ml. In some embodiments, the final concentration of the cytokine IL-7 in the cell culture media is 700 ng/ml to 800 ng/ml. In some embodiments, the final concentration of the cytokine IL-7 in the cell culture media is 800 ng/ml to 900 ng/ml. In some embodiments, the final concentration of the cytokine IL-7 in the cell culture media is 900 ng/ml to 1000 ng/ml. In some embodiments, the final concentration of the cytokine IL-7 in the cell culture media is 1,000 ng/ml to 1,500 ng/ml. In some embodiments, the final concentration of the cytokine IL-7 in the cell culture media is 1,500 ng/ml to 2,000 ng/ml. In some embodiments, the final concentration of the cytokine IL-7 in the cell culture media is 2,000 ng/ml to 2,500 ng/ml. In some embodiments, the final concentration of the cytokine IL-7 in the cell culture media is 2,500 ng/ml to 3,000 ng/ml. In some embodiments, the final concentration of the cytokine IL-7 in the cell culture media is 3,000 ng/ml to 3,500 ng/ml. In some embodiments, the final concentration of the cytokine IL-7 in the cell culture media is 3,500 ng/ml to 4,000 ng/ml. In some embodiments, the final concentration of the cytokine IL-7 in the cell culture media is 4,000 ng/ml to 4,500 ng/ml. In some embodiments, the final concentration of the cytokine IL-7 in the cell culture media is 4,500 ng/ml to 5,000 ng/ml. In some embodiments, the final concentration of the cytokine IL-7 in the cell culture media is 5,000 ng/ml to 5,500 ng/ml. In some embodiments, the final concentration of the cytokine IL-7 in the cell culture media is 5,500 ng/ml to 6,000 ng/ml. In some embodiments, the final concentration of the cytokine IL-7 in the cell culture media is 6,000 ng/ml to 6,500 ng/ml. In some embodiments, the final concentration of the cytokine IL-7 in the cell culture media is 6,500 ng/ml to 7,000 ng/ml. In some embodiments, the final concentration of the cytokine IL-7 in the cell culture media is 7,000 ng/ml to 7,500 ng/ml. In some embodiments, the final concentration of the cytokine IL-7 in the cell culture media is 7,500 ng/ml to 8,000 ng/ml. In some embodiments, the final concentration of the cytokine IL-7 in the cell culture media is 8,000 ng/ml to 8,500 ng/ml. In some embodiments, the final concentration of the cytokine IL-7 in the cell culture media is 8,500 ng/ml to 9,000 ng/ml. In some embodiments, the final concentration of the cytokine IL-7 in the cell culture media is 9,000 ng/ml to 9,500 ng/ml. In some embodiments, the final concentration of the cytokine IL-7 in the cell culture media is 9,500 ng/ml to 10,000 ng/ml.

In some embodiments, the final concentration of the cytokine IL-7 in the cell culture media is 1 U/ml to 20,000 U/ml. In some embodiments, the final concentration of the cytokine IL-7 in the cell culture media is 2 U/ml to 20,000 U/ml. In some embodiments, the final concentration of the cytokine IL-7 in the cell culture media is 20 U/ml to 20,000 U/ml. In some embodiments, the final concentration of the cytokine IL-7 in the cell culture media is 2 U/ml to 20 U/ml. In some embodiments, the final concentration of the cytokine IL-7 in the cell culture media is 20 U/ml to 50 U/ml. In some embodiments, the final concentration of the cytokine IL-7 in the cell culture media is 50 U/ml to 100 U/ml. In some embodiments, the final concentration of the cytokine IL-7 in the cell culture media is 100 Um′ to 150 U/ml. In some embodiments, the final concentration of the cytokine IL-7 in the cell culture media is 150 Um′ to 200 U/ml. In some embodiments, the final concentration of the cytokine IL-7 in the cell culture media is 200 U/ml to 400 U/ml. In some embodiments, the final concentration of the cytokine IL-7 in the cell culture media is 400 U/ml to 600 U/ml. In some embodiments, the final concentration of the cytokine IL-7 in the cell culture media is 600 U/ml to 800 U/ml. In some embodiments, the final concentration of the cytokine IL-7 in the cell culture media is 800 U/ml to 1000 U/ml. In some embodiments, the final concentration of the cytokine IL-7 in the cell culture media is 1000 U/ml to 1200 U/ml. In some embodiments, the final concentration of the cytokine IL-7 in the cell culture media is 1200 U/ml to 1400 U/ml. In some embodiments, the final concentration of the cytokine IL-7 in the cell culture media is 1400 Um′ to 1600 U/ml. In some embodiments, the final concentration of the cytokine IL-7 in the cell culture media is 1600 U/ml to 1800 U/ml. In some embodiments, the final concentration of the cytokine IL-7 in the cell culture media is 1800 U/ml to 2000 U/ml. In some embodiments, the final concentration of the cytokine IL-7 in the cell culture media is 2000 U/ml to 3000 U/ml. In some embodiments, the final concentration of the cytokine IL-7 in the cell culture media is 3000 U/ml to 4000 U/ml. In some embodiments, the final concentration of the cytokine IL-7 in the cell culture media is 4000 U/ml to 5000 U/ml. In some embodiments, the final concentration of the cytokine IL-7 in the cell culture media is 5000 U/ml to 6000 U/ml. In some embodiments, the final concentration of the cytokine IL-7 in the cell culture media is 6000 U/ml to 7000 U/ml. In some embodiments, the final concentration of the cytokine IL-7 in the cell culture media is 7000 U/ml to 8000 U/ml. In some embodiments, the final concentration of the cytokine IL-7 in the cell culture media is 8000 U/ml to 9000 U/ml. In some embodiments, the final concentration of the cytokine IL-7 in the cell culture media is 9000 U/ml to 10,000 U/ml. In some embodiments, the final concentration of the cytokine IL-7 in the cell culture media is 10,000 U/ml to 11,000 U/ml. In some embodiments, the final concentration of the cytokine IL-7 in the cell culture media is 11,000 U/ml to 12,000 U/ml. In some embodiments, the final concentration of the cytokine IL-7 in the cell culture media is 12,000 U/ml to 13,000 U/ml. In some embodiments, the final concentration of the cytokine IL-7 in the cell culture media is 13,000 U/ml to 14,000 U/ml. In some embodiments, the final concentration of the cytokine IL-7 in the cell culture media is 14,000 U/ml to 15,000 U/ml. In some embodiments, the final concentration of the cytokine IL-7 in the cell culture media is 15,000 U/ml to 16,000 U/ml. In some embodiments, the final concentration of the cytokine IL-7 in the cell culture media is 16,000 U/ml to 17,000 U/ml. In some embodiments, the final concentration of the cytokine IL-7 in the cell culture media is 17,000 U/ml to 18,000 U/ml. In some embodiments, the final concentration of the cytokine IL-7 in the cell culture media is 18,000 U/ml to 19,000 U/ml. In some embodiments, the final concentration of the cytokine IL-7 in the cell culture media is 19,000 U/ml to 20,000 U/ml.

In some embodiments, the methods disclosed herein comprise contacting the electroporated T cells with the cytokine IL-12. In some embodiments, the T cells are contacted with the cytokine IL-12 every other day. In some embodiments, the T cells are contacted with the cytokine IL-12 in time intervals of 2, 3, 4, 5, or 6 days. In some embodiments, the T cells are contacted with the cytokine IL-12 in a time interval of 2 days. In some embodiments, the T cells are contacted with the cytokine IL-12 in a time interval of 3 days. In some embodiments, the T cells are contacted with the cytokine IL-12 in a time interval of 4 days. In some embodiments, the T cells are contacted with the cytokine IL-12 in a time interval of 5 days. In some embodiments, the T cells are contacted with the cytokine IL-12 in a time interval of 6 days.

In some embodiments, the final concentration of the cytokine IL-12 in the cell culture media is 0.5 ng/ml to 10,000 ng/ml. In some embodiments, the final concentration of the cytokine IL-12 in the cell culture media is 10 ng/ml to 10,000 ng/ml. In some embodiments, the final concentration of the cytokine IL-12 in the cell culture media is 0.5 ng/ml to 10 ng/ml. In some embodiments, the final concentration of the cytokine IL-12 in the cell culture media is 10 ng/ml to 25 ng/ml. In some embodiments, the final concentration of the cytokine IL-12 in the cell culture media is 25 ng/ml to 50 ng/ml. In some embodiments, the final concentration of the cytokine IL-12 in the cell culture media is 50 ng/ml to 75 ng/ml. In some embodiments, the final concentration of the cytokine IL-12 in the cell culture media is 75 ng/ml to 100 ng/ml. In some embodiments, the final concentration of the cytokine IL-12 in the cell culture media is 100 ng/ml to 200 ng/ml. In some embodiments, the final concentration of the cytokine IL-12 in the cell culture media is 200 ng/ml to 300 ng/ml. In some embodiments, the final concentration of the cytokine IL-12 in the cell culture media is 300 ng/ml to 400 ng/ml. In some embodiments, the final concentration of the cytokine IL-12 in the cell culture media is 400 ng/ml to 500 ng/ml. In some embodiments, the final concentration of the cytokine IL-12 in the cell culture media is 500 ng/ml to 600 ng/ml. In some embodiments, the final concentration of the cytokine IL-12 in the cell culture media is 600 ng/ml to 700 ng/ml. In some embodiments, the final concentration of the cytokine IL-12 in the cell culture media is 700 ng/ml to 800 ng/ml. In some embodiments, the final concentration of the cytokine IL-12 in the cell culture media is 800 ng/ml to 900 ng/ml. In some embodiments, the final concentration of the cytokine IL-12 in the cell culture media is 900 ng/ml to 1000 ng/ml. In some embodiments, the final concentration of the cytokine IL-12 in the cell culture media is 1,000 ng/ml to 1,500 ng/ml. In some embodiments, the final concentration of the cytokine IL-12 in the cell culture media is 1,500 ng/ml to 2,000 ng/ml. In some embodiments, the final concentration of the cytokine IL-12 in the cell culture media is 2,000 ng/ml to 2,500 ng/ml. In some embodiments, the final concentration of the cytokine IL-12 in the cell culture media is 2,500 ng/ml to 3,000 ng/ml. In some embodiments, the final concentration of the cytokine IL-12 in the cell culture media is 3,000 ng/ml to 3,500 ng/ml. In some embodiments, the final concentration of the cytokine IL-12 in the cell culture media is 3,500 ng/ml to 4,000 ng/ml. In some embodiments, the final concentration of the cytokine IL-12 in the cell culture media is 4,000 ng/ml to 4,500 ng/ml. In some embodiments, the final concentration of the cytokine IL-12 in the cell culture media is 4,500 ng/ml to 5,000 ng/ml. In some embodiments, the final concentration of the cytokine IL-12 in the cell culture media is 5,000 ng/ml to 5,500 ng/ml. In some embodiments, the final concentration of the cytokine IL-12 in the cell culture media is 5,500 ng/ml to 6,000 ng/ml. In some embodiments, the final concentration of the cytokine IL-12 in the cell culture media is 6,000 ng/ml to 6,500 ng/ml. In some embodiments, the final concentration of the cytokine IL-12 in the cell culture media is 6,500 ng/ml to 7,000 ng/ml. In some embodiments, the final concentration of the cytokine IL-12 in the cell culture media is 7,000 ng/ml to 7,500 ng/ml. In some embodiments, the final concentration of the cytokine IL-12 in the cell culture media is 7,500 ng/ml to 8,000 ng/ml. In some embodiments, the final concentration of the cytokine IL-12 in the cell culture media is 8,000 ng/ml to 8,500 ng/ml. In some embodiments, the final concentration of the cytokine IL-12 in the cell culture media is 8,500 ng/ml to 9,000 ng/ml. In some embodiments, the final concentration of the cytokine IL-12 in the cell culture media is 9,000 ng/ml to 9,500 ng/ml. In some embodiments, the final concentration of the cytokine IL-12 in the cell culture media is 9,500 ng/ml to 10,000 ng/ml.

In some embodiments, the final concentration of the cytokine IL-12 in the cell culture media is 1 U/ml to 20,000 U/ml. In some embodiments, the final concentration of the cytokine IL-12 in the cell culture media is 2 U/ml to 20,000 U/ml. In some embodiments, the final concentration of the cytokine IL-12 in the cell culture media is 20 U/ml to 20,000 U/ml. In some embodiments, the final concentration of the cytokine IL-12 in the cell culture media is 2 U/ml to 20 U/ml. In some embodiments, the final concentration of the cytokine IL-12 in the cell culture media is 20 U/ml to 50 U/ml. In some embodiments, the final concentration of the cytokine IL-12 in the cell culture media is 50 U/ml to 100 U/ml. In some embodiments, the final concentration of the cytokine IL-12 in the cell culture media is 100 U/ml to 150 U/ml. In some embodiments, the final concentration of the cytokine IL-12 in the cell culture media is 150 U/ml to 200 U/ml. In some embodiments, the final concentration of the cytokine IL-12 in the cell culture media is 200 U/ml to 400 U/ml. In some embodiments, the final concentration of the cytokine IL-12 in the cell culture media is 400 U/ml to 600 U/ml. In some embodiments, the final concentration of the cytokine IL-12 in the cell culture media is 600 U/ml to 800 U/ml. In some embodiments, the final concentration of the cytokine IL-12 in the cell culture media is 800 U/ml to 1000 U/ml. In some embodiments, the final concentration of the cytokine IL-12 in the cell culture media is 1000 U/ml to 1200 U/ml. In some embodiments, the final concentration of the cytokine IL-12 in the cell culture media is 1200 U/ml to 1400 U/ml. In some embodiments, the final concentration of the cytokine IL-12 in the cell culture media is 1400 U/ml to 1600 U/ml. In some embodiments, the final concentration of the cytokine IL-12 in the cell culture media is 1600 U/ml to 1800 U/ml. In some embodiments, the final concentration of the cytokine IL-12 in the cell culture media is 1800 U/ml to 2000 U/ml. In some embodiments, the final concentration of the cytokine IL-12 in the cell culture media is 2000 U/ml to 3000 U/ml. In some embodiments, the final concentration of the cytokine IL-12 in the cell culture media is 3000 U/ml to 4000 U/ml. In some embodiments, the final concentration of the cytokine IL-12 in the cell culture media is 4000 U/ml to 5000 U/ml. In some embodiments, the final concentration of the cytokine IL-12 in the cell culture media is 5000 U/ml to 6000 U/ml. In some embodiments, the final concentration of the cytokine IL-12 in the cell culture media is 6000 U/ml to 7000 U/ml. In some embodiments, the final concentration of the cytokine IL-12 in the cell culture media is 7000 U/ml to 8000 U/ml. In some embodiments, the final concentration of the cytokine IL-12 in the cell culture media is 8000 U/ml to 9000 U/ml. In some embodiments, the final concentration of the cytokine IL-12 in the cell culture media is 9000 U/ml to 10,000 U/ml. In some embodiments, the final concentration of the cytokine IL-12 in the cell culture media is 10,000 U/ml to 11,000 U/ml. In some embodiments, the final concentration of the cytokine IL-12 in the cell culture media is 11,000 U/ml to 12,000 U/ml. In some embodiments, the final concentration of the cytokine IL-12 in the cell culture media is 12,000 U/ml to 13,000 U/ml. In some embodiments, the final concentration of the cytokine IL-12 in the cell culture media is 13,000 U/ml to 14,000 U/ml. In some embodiments, the final concentration of the cytokine IL-12 in the cell culture media is 14,000 U/ml to 15,000 U/ml. In some embodiments, the final concentration of the cytokine IL-12 in the cell culture media is 15,000 U/ml to 16,000 U/ml. In some embodiments, the final concentration of the cytokine IL-12 in the cell culture media is 16,000 U/ml to 17,000 U/ml. In some embodiments, the final concentration of the cytokine IL-12 in the cell culture media is 17,000 U/ml to 18,000 U/ml. In some embodiments, the final concentration of the cytokine IL-12 in the cell culture media is 18,000 U/ml to 19,000 U/ml. In some embodiments, the final concentration of the cytokine IL-12 in the cell culture media is 19,000 U/ml to 20,000 U/ml.

In some embodiments, the methods disclosed herein comprise contacting the electroporated T cells with the cytokine IL-21. In some embodiments, the T cells are contacted with the cytokine IL-21 every other day. In some embodiments, the T cells are contacted with the cytokine IL-21 in time intervals of 2, 3, 4, 5, or 6 days. In some embodiments, the T cells are contacted with the cytokine IL-21 in a time interval of 2 days. In some embodiments, the T cells are contacted with the cytokine IL-21 in a time interval of 3 days. In some embodiments, the T cells are contacted with the cytokine IL-21 in a time interval of 4 days. In some embodiments, the T cells are contacted with the cytokine IL-21 in a time interval of 5 days. In some embodiments, the T cells are contacted with the cytokine IL-21 in a time interval of 6 days.

In some embodiments, the final concentration of the cytokine IL-21 in the cell culture media is 0.5 ng/ml to 10,000 ng/ml. In some embodiments, the final concentration of the cytokine IL-21 in the cell culture media is 10 ng/ml to 10,000 ng/ml. In some embodiments, the final concentration of the cytokine IL-21 in the cell culture media is 0.5 ng/ml to 10 ng/ml. In some embodiments, the final concentration of the cytokine IL-21 in the cell culture media is 10 ng/ml to 25 ng/ml. In some embodiments, the final concentration of the cytokine IL-21 in the cell culture media is 25 ng/ml to 50 ng/ml. In some embodiments, the final concentration of the cytokine IL-21 in the cell culture media is 50 ng/ml to 75 ng/ml. In some embodiments, the final concentration of the cytokine IL-21 in the cell culture media is 75 ng/ml to 100 ng/ml. In some embodiments, the final concentration of the cytokine IL-21 in the cell culture media is 100 ng/ml to 200 ng/ml. In some embodiments, the final concentration of the cytokine IL-21 in the cell culture media is 200 ng/ml to 300 ng/ml. In some embodiments, the final concentration of the cytokine IL-21 in the cell culture media is 300 ng/ml to 400 ng/ml. In some embodiments, the final concentration of the cytokine IL-21 in the cell culture media is 400 ng/ml to 500 ng/ml. In some embodiments, the final concentration of the cytokine IL-21 in the cell culture media is 500 ng/ml to 600 ng/ml. In some embodiments, the final concentration of the cytokine IL-21 in the cell culture media is 600 ng/ml to 700 ng/ml. In some embodiments, the final concentration of the cytokine IL-21 in the cell culture media is 700 ng/ml to 800 ng/ml. In some embodiments, the final concentration of the cytokine IL-21 in the cell culture media is 800 ng/ml to 900 ng/ml. In some embodiments, the final concentration of the cytokine IL-21 in the cell culture media is 900 ng/ml to 1000 ng/ml. In some embodiments, the final concentration of the cytokine IL-21 in the cell culture media is 1,000 ng/ml to 1,500 ng/ml. In some embodiments, the final concentration of the cytokine IL-21 in the cell culture media is 1,500 ng/ml to 2,000 ng/ml. In some embodiments, the final concentration of the cytokine IL-21 in the cell culture media is 2,000 ng/ml to 2,500 ng/ml. In some embodiments, the final concentration of the cytokine IL-21 in the cell culture media is 2,500 ng/ml to 3,000 ng/ml. In some embodiments, the final concentration of the cytokine IL-21 in the cell culture media is 3,000 ng/ml to 3,500 ng/ml. In some embodiments, the final concentration of the cytokine IL-21 in the cell culture media is 3,500 ng/ml to 4,000 ng/ml. In some embodiments, the final concentration of the cytokine IL-21 in the cell culture media is 4,000 ng/ml to 4,500 ng/ml. In some embodiments, the final concentration of the cytokine IL-21 in the cell culture media is 4,500 ng/ml to 5,000 ng/ml. In some embodiments, the final concentration of the cytokine IL-21 in the cell culture media is 5,000 ng/ml to 5,500 ng/ml. In some embodiments, the final concentration of the cytokine IL-21 in the cell culture media is 5,500 ng/ml to 6,000 ng/ml. In some embodiments, the final concentration of the cytokine IL-21 in the cell culture media is 6,000 ng/ml to 6,500 ng/ml. In some embodiments, the final concentration of the cytokine IL-21 in the cell culture media is 6,500 ng/ml to 7,000 ng/ml. In some embodiments, the final concentration of the cytokine IL-21 in the cell culture media is 7,000 ng/ml to 7,500 ng/ml. In some embodiments, the final concentration of the cytokine IL-21 in the cell culture media is 7,500 ng/ml to 8,000 ng/ml. In some embodiments, the final concentration of the cytokine IL-21 in the cell culture media is 8,000 ng/ml to 8,500 ng/ml. In some embodiments, the final concentration of the cytokine IL-21 in the cell culture media is 8,500 ng/ml to 9,000 ng/ml. In some embodiments, the final concentration of the cytokine IL-21 in the cell culture media is 9,000 ng/ml to 9,500 ng/ml. In some embodiments, the final concentration of the cytokine IL-21 in the cell culture media is 9,500 ng/ml to 10,000 ng/ml.

In some embodiments, the final concentration of the cytokine IL-21 in the cell culture media is 1 U/ml to 20,000 U/ml. In some embodiments, the final concentration of the cytokine IL-21 in the cell culture media is 2 U/ml to 20,000 U/ml. In some embodiments, the final concentration of the cytokine IL-21 in the cell culture media is 20 U/ml to 20,000 U/ml. In some embodiments, the final concentration of the cytokine IL-21 in the cell culture media is 2 U/ml to 20 U/ml. In some embodiments, the final concentration of the cytokine IL-21 in the cell culture media is 20 U/ml to 50 U/ml. In some embodiments, the final concentration of the cytokine IL-21 in the cell culture media is 50 U/ml to 100 U/ml. In some embodiments, the final concentration of the cytokine IL-21 in the cell culture media is 100 U/ml to 150 U/ml. In some embodiments, the final concentration of the cytokine IL-21 in the cell culture media is 150 U/ml to 200 U/ml. In some embodiments, the final concentration of the cytokine IL-21 in the cell culture media is 200 U/ml to 400 U/ml. In some embodiments, the final concentration of the cytokine IL-21 in the cell culture media is 400 U/ml to 600 U/ml. In some embodiments, the final concentration of the cytokine IL-21 in the cell culture media is 600 U/ml to 800 U/ml. In some embodiments, the final concentration of the cytokine IL-21 in the cell culture media is 800 U/ml to 1000 U/ml. In some embodiments, the final concentration of the cytokine IL-21 in the cell culture media is 1000 U/ml to 1200 U/ml. In some embodiments, the final concentration of the cytokine IL-21 in the cell culture media is 1200 U/ml to 1400 U/ml. In some embodiments, the final concentration of the cytokine IL-21 in the cell culture media is 1400 U/ml to 1600 U/ml. In some embodiments, the final concentration of the cytokine IL-21 in the cell culture media is 1600 U/ml to 1800 U/ml. In some embodiments, the final concentration of the cytokine IL-21 in the cell culture media is 1800 U/ml to 2000 U/ml. In some embodiments, the final concentration of the cytokine IL-21 in the cell culture media is 2000 U/ml to 3000 U/ml. In some embodiments, the final concentration of the cytokine IL-21 in the cell culture media is 3000 U/ml to 4000 U/ml. In some embodiments, the final concentration of the cytokine IL-21 in the cell culture media is 4000 U/ml to 5000 U/ml. In some embodiments, the final concentration of the cytokine IL-21 in the cell culture media is 5000 U/ml to 6000 U/ml. In some embodiments, the final concentration of the cytokine IL-21 in the cell culture media is 6000 U/ml to 7000 U/ml. In some embodiments, the final concentration of the cytokine IL-21 in the cell culture media is 7000 U/ml to 8000 U/ml. In some embodiments, the final concentration of the cytokine IL-21 in the cell culture media is 8000 U/ml to 9000 U/ml. In some embodiments, the final concentration of the cytokine IL-21 in the cell culture media is 9000 U/ml to 10,000 U/ml. In some embodiments, the final concentration of the cytokine IL-21 in the cell culture media is 10,000 U/ml to 11,000 U/ml. In some embodiments, the final concentration of the cytokine IL-21 in the cell culture media is 11,000 U/ml to 12,000 U/ml. In some embodiments, the final concentration of the cytokine IL-21 in the cell culture media is 12,000 U/ml to 13,000 U/ml. In some embodiments, the final concentration of the cytokine IL-21 in the cell culture media is 13,000 U/ml to 14,000 U/ml. In some embodiments, the final concentration of the cytokine IL-21 in the cell culture media is 14,000 U/ml to 15,000 U/ml. In some embodiments, the final concentration of the cytokine IL-21 in the cell culture media is 15,000 U/ml to 16,000 U/ml. In some embodiments, the final concentration of the cytokine IL-21 in the cell culture media is 16,000 U/ml to 17,000 U/ml. In some embodiments, the final concentration of the cytokine IL-21 in the cell culture media is 17,000 U/ml to 18,000 U/ml. In some embodiments, the final concentration of the cytokine IL-21 in the cell culture media is 18,000 U/ml to 19,000 U/ml. In some embodiments, the final concentration of the cytokine IL-21 in the cell culture media is 19,000 U/ml to 20,000 U/ml.

In some embodiments, the methods disclosed herein comprise contacting the electroporated T cells with the cytokine IL-2. In some embodiments, the T cells are contacted with the cytokine IL-2 every other day. In some embodiments, the T cells are contacted with the cytokine IL-2 in time intervals of 2, 3, 4, 5, or 6 days. In some embodiments, the T cells are contacted with the cytokine IL-2 in a time interval of 2 days. In some embodiments, the T cells are contacted with the cytokine IL-2 in a time interval of 3 days. In some embodiments, the T cells are contacted with the cytokine IL-2 in a time interval of 4 days. In some embodiments, the T cells are contacted with the cytokine IL-2 in a time interval of 5 days. In some embodiments, the T cells are contacted with the cytokine IL-2 in a time interval of 6 days.

In some embodiments, the final concentration of the cytokine IL-2 in the cell culture media is 0.50 ng/ml to 10,000 ng/ml. In some embodiments, the final concentration of the cytokine IL-2 in the cell culture media is 10 ng/ml to 10,000 ng/ml. In some embodiments, the final concentration of the cytokine IL-2 in the cell culture media is 0.5 ng/ml to 10 ng/ml. In some embodiments, the final concentration of the cytokine IL-2 in the cell culture media is 10 ng/ml to 25 ng/ml. In some embodiments, the final concentration of the cytokine IL-2 in the cell culture media is 25 ng/ml to 50 ng/ml. In some embodiments, the final concentration of the cytokine IL-2 in the cell culture media is 50 ng/ml to 75 ng/ml. In some embodiments, the final concentration of the cytokine IL-2 in the cell culture media is 75 ng/ml to 100 ng/ml. In some embodiments, the final concentration of the cytokine IL-2 in the cell culture media is 100 ng/ml to 200 ng/ml. In some embodiments, the final concentration of the cytokine IL-2 in the cell culture media is 200 ng/ml to 300 ng/ml. In some embodiments, the final concentration of the cytokine IL-2 in the cell culture media is 300 ng/ml to 400 ng/ml. In some embodiments, the final concentration of the cytokine IL-2 in the cell culture media is 400 ng/ml to 500 ng/ml. In some embodiments, the final concentration of the cytokine IL-2 in the cell culture media is 500 ng/ml to 600 ng/ml. In some embodiments, the final concentration of the cytokine IL-2 in the cell culture media is 600 ng/ml to 700 ng/ml. In some embodiments, the final concentration of the cytokine IL-2 in the cell culture media is 700 ng/ml to 800 ng/ml. In some embodiments, the final concentration of the cytokine IL-2 in the cell culture media is 800 ng/ml to 900 ng/ml. In some embodiments, the final concentration of the cytokine IL-2 in the cell culture media is 900 ng/ml to 1000 ng/ml. In some embodiments, the final concentration of the cytokine IL-2 in the cell culture media is 1,000 ng/ml to 1,500 ng/ml. In some embodiments, the final concentration of the cytokine IL-2 in the cell culture media is 1,500 ng/ml to 2,000 ng/ml. In some embodiments, the final concentration of the cytokine IL-2 in the cell culture media is 2,000 ng/ml to 2,500 ng/ml. In some embodiments, the final concentration of the cytokine IL-2 in the cell culture media is 2,500 ng/ml to 3,000 ng/ml. In some embodiments, the final concentration of the cytokine IL-2 in the cell culture media is 3,000 ng/ml to 3,500 ng/ml. In some embodiments, the final concentration of the cytokine IL-2 in the cell culture media is 3,500 ng/ml to 4,000 ng/ml. In some embodiments, the final concentration of the cytokine IL-2 in the cell culture media is 4,000 ng/ml to 4,500 ng/ml. In some embodiments, the final concentration of the cytokine IL-2 in the cell culture media is 4,500 ng/ml to 5,000 ng/ml. In some embodiments, the final concentration of the cytokine IL-2 in the cell culture media is 5,000 ng/ml to 5,500 ng/ml. In some embodiments, the final concentration of the cytokine IL-2 in the cell culture media is 5,500 ng/ml to 6,000 ng/ml. In some embodiments, the final concentration of the cytokine IL-2 in the cell culture media is 6,000 ng/ml to 6,500 ng/ml. In some embodiments, the final concentration of the cytokine IL-2 in the cell culture media is 6,500 ng/ml to 7,000 ng/ml. In some embodiments, the final concentration of the cytokine IL-2 in the cell culture media is 7,000 ng/ml to 7,500 ng/ml. In some embodiments, the final concentration of the cytokine IL-2 in the cell culture media is 7,500 ng/ml to 8,000 ng/ml. In some embodiments, the final concentration of the cytokine IL-2 in the cell culture media is 8,000 ng/ml to 8,500 ng/ml. In some embodiments, the final concentration of the cytokine IL-2 in the cell culture media is 8,500 ng/ml to 9,000 ng/ml. In some embodiments, the final concentration of the cytokine IL-2 in the cell culture media is 9,000 ng/ml to 9,500 ng/ml. In some embodiments, the final concentration of the cytokine IL-2 in the cell culture media is 9,500 ng/ml to 10,000 ng/ml.

In some embodiments, the final concentration of the cytokine IL-2 in the cell culture media is 1 U/ml to 20,000 U/ml. In some embodiments, the final concentration of the cytokine IL-2 in the cell culture media is 2 U/ml to 20,000 U/ml. In some embodiments, the final concentration of the cytokine IL-2 in the cell culture media is 20 U/ml to 20,000 U/ml. In some embodiments, the final concentration of the cytokine IL-2 in the cell culture media is 2 U/ml to 20 U/ml. In some embodiments, the final concentration of the cytokine IL-2 in the cell culture media is 20 U/ml to 50 U/ml. In some embodiments, the final concentration of the cytokine IL-2 in the cell culture media is 50 U/ml to 100 U/ml. In some embodiments, the final concentration of the cytokine IL-2 in the cell culture media is 100 Um′ to 150 U/ml. In some embodiments, the final concentration of the cytokine IL-2 in the cell culture media is 150 Um′ to 200 U/ml. In some embodiments, the final concentration of the cytokine IL-2 in the cell culture media is 200 U/ml to 400 U/ml. In some embodiments, the final concentration of the cytokine IL-2 in the cell culture media is 400 U/ml to 600 U/ml. In some embodiments, the final concentration of the cytokine IL-2 in the cell culture media is 600 U/ml to 800 U/ml. In some embodiments, the final concentration of the cytokine IL-2 in the cell culture media is 800 U/ml to 1000 U/ml. In some embodiments, the final concentration of the cytokine IL-2 in the cell culture media is 1000 U/ml to 1200 U/ml. In some embodiments, the final concentration of the cytokine IL-2 in the cell culture media is 1200 U/ml to 1400 U/ml. In some embodiments, the final concentration of the cytokine IL-2 in the cell culture media is 1400 Um′ to 1600 U/ml. In some embodiments, the final concentration of the cytokine IL-2 in the cell culture media is 1600 U/ml to 1800 U/ml. In some embodiments, the final concentration of the cytokine IL-2 in the cell culture media is 1800 U/ml to 2000 U/ml. In some embodiments, the final concentration of the cytokine IL-2 in the cell culture media is 2000 U/ml to 3000 U/ml. In some embodiments, the final concentration of the cytokine IL-2 in the cell culture media is 3000 U/ml to 4000 U/ml. In some embodiments, the final concentration of the cytokine IL-2 in the cell culture media is 4000 U/ml to 5000 U/ml. In some embodiments, the final concentration of the cytokine IL-2 in the cell culture media is 5000 U/ml to 6000 U/ml. In some embodiments, the final concentration of the cytokine IL-2 in the cell culture media is 6000 U/ml to 7000 U/ml. In some embodiments, the final concentration of the cytokine IL-2 in the cell culture media is 7000 U/ml to 8000 U/ml. In some embodiments, the final concentration of the cytokine IL-2 in the cell culture media is 8000 U/ml to 9000 U/ml. In some embodiments, the final concentration of the cytokine IL-2 in the cell culture media is 9000 U/ml to 10,000 U/ml. In some embodiments, the final concentration of the cytokine IL-2 in the cell culture media is 10,000 U/ml to 11,000 U/ml. In some embodiments, the final concentration of the cytokine IL-2 in the cell culture media is 11,000 U/ml to 12,000 U/ml. In some embodiments, the final concentration of the cytokine IL-2 in the cell culture media is 12,000 U/ml to 13,000 U/ml. In some embodiments, the final concentration of the cytokine IL-2 in the cell culture media is 13,000 U/ml to 14,000 U/ml. In some embodiments, the final concentration of the cytokine IL-2 in the cell culture media is 14,000 U/ml to 15,000 U/ml. In some embodiments, the final concentration of the cytokine IL-2 in the cell culture media is 15,000 U/ml to 16,000 U/ml. In some embodiments, the final concentration of the cytokine IL-2 in the cell culture media is 16,000 U/ml to 17,000 U/ml. In some embodiments, the final concentration of the cytokine IL-2 in the cell culture media is 17,000 U/ml to 18,000 U/ml. In some embodiments, the final concentration of the cytokine IL-2 in the cell culture media is 18,000 U/ml to 19,000 U/ml. In some embodiments, the final concentration of the cytokine IL-2 in the cell culture media is 19,000 U/ml to 20,000 U/ml.

In some embodiments, the T cell stimulating cytokines are only administered to a culture media at the first day of administering the culture media to cells, but not readministered when the media is changed. In some embodiments, IL-7 is provided only on day 1 of culture. In some embodiments, IL-12 is provided only on day 1 of culture.

b. Methods of Use

In another aspect, the instant disclosure provides a method of treating a subject using the polycistronic polynucleotides, recombinant vectors, engineered cells (e.g., a cell comprising a heterologous and/or recombinant nucleic acid), or pharmaceutical compositions disclosed herein. Any disease or disorder in a subject that would benefit from treatment with a recombinant cell of the present disclosure, or a polynucleotide or vector of the present disclosure can be treated using the methods disclosed herein.

In certain embodiments, the method comprises administering to the subject an effective amount of a recombinant cell or population thereof as disclosed herein.

As disclosed infra, cells administered to the subject can be autologous or allogeneic to the subject. In certain embodiments, autologous cells are obtained from a cancer patient directly following a cancer treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, in certain embodiments, cells are collected from blood, bone marrow, lymph node, thymus, or another tissue or bodily fluid, or an apheresis product, during this recovery phase. Further, in certain aspects, mobilization and conditioning regimens can be used to create a condition in a subject wherein repopulation, recirculation, regeneration, and/or expansion of particular cell types is favored, especially during a defined window of time following therapy.

The number of cells that are employed will depend upon a number of circumstances including, the lifetime of the cells, the protocol to be used (e.g., the number of administrations), the ability of the cells to multiply, the stability of the recombinant construct, and the like. In certain embodiments, the cells are applied as a dispersion, generally being injected at or near the site of interest. The cells may be administered in any physiologically acceptable medium.

In certain embodiments, the cancer is cancer of the lung, bile duct cancer (e.g., cholangiocarcinoma), pancreatic cancer, colorectal cancer, ovarian, or gynecologic cancer. In certain embodiments, the cancer is leukemia (e.g., mixed lineage leukemia, acute lymphocytic leukemia, acute myeloid leukemia, chronic lymphocytic leukemia, or chronic myeloid leukemia), alveolar rhabdomyosarcoma, bone cancer, brain cancer (e.g., glioma, e.g., glioblastoma), breast cancer, cancer of the anus, anal canal, or anorectum, cancer of the eye, cancer of the intrahepatic bile duct (e.g., intrahepatic cholangiocellular cancer), cancer of the joints, cancer of the neck, gallbladder, or pleura, cancer of the nose, nasal cavity, or middle ear, cancer of the oral cavity, cancer of the vulva, myeloma (e.g., chronic myeloid cancer), colon cancer, esophageal cancer, cervical cancer, gastrointestinal cancer, gastrointestinal carcinoid tumor, Hodgkin's lymphoma, hypopharynx cancer, kidney cancer, larynx cancer, liver cancer (e.g., hepatocellular carcinoma), lung cancer (e.g., non-small cell lung cancer), malignant mesothelioma, melanoma, multiple myeloma, nasopharynx cancer, non-Hodgkin's lymphoma, ovarian cancer, pancreatic cancer, peritoneum, omentum, and mesentery cancer, pharynx cancer, prostate cancer, rectal cancer, renal cancer (e.g., renal cell carcinoma (RCC)), gastric cancer, small intestine cancer, soft tissue cancer, stomach cancer, carcinoma, sarcoma (e.g., synovial sarcoma, rhabdomyosarcoma), skin cancer, testicular cancer, thyroid cancer, head and neck cancer, ureter cancer, and urinary bladder cancer. In certain embodiments, the cancer is melanoma, breast cancer, lung cancer, prostate cancer, thyroid cancer, ovarian cancer, or synovial sarcoma. In one embodiment, the cancer is synovial sarcoma or liposarcoma (e.g., myxoid/round cell liposarcoma). In certain embodiments, the cancer is lung, cholangiocarcinoma, pancreatic, colorectal, gynecological or ovarian cancer.

A polycistronic polynucleotide, recombinant vector, engineered cell, or pharmaceutical composition described herein may be delivered to a subject by a variety of routes. These include, but are not limited to, parenteral, intranasal, intratracheal, oral, intradermal, topical, intramuscular, intraperitoneal, transdermal, intravenous, intratumoral, conjunctival, intrathecal, and subcutaneous routes. Pulmonary administration can also be employed, e.g., by use of an inhaler or nebulizer, and formulation with an aerosolizing agent for use as a spray. In certain embodiments, the polycistronic polynucleotide, recombinant vector, engineered cell, or pharmaceutical composition described herein is delivered intravenously. In certain embodiments, the polycistronic polynucleotide, vector, engineered cell, or pharmaceutical composition described herein is delivered subcutaneously. In certain embodiments, the polycistronic polynucleotide, recombinant vector, engineered cell, or pharmaceutical composition described herein is delivered intratumorally. In certain embodiments, the polycistronic polynucleotide, recombinant vector, engineered cell, or pharmaceutical composition described herein is delivered into a tumor draining lymph node.

The amount of the polycistronic polynucleotide, recombinant vector, engineered cell, or pharmaceutical composition which will be effective in the treatment and/or prevention of a condition will depend on the nature of the disease, and can be determined by standard clinical techniques.

The precise dose to be employed in a composition will also depend on various factors, including but not limited to the route of administration, and the seriousness of the infection or disease caused by it, and should be decided according to the judgment of the practitioner and each subject's circumstances. For example, effective doses may also vary depending upon means of administration, target site, physiological state of the patient (including age, body weight, and health), whether the patient is a human or an animal, other medications administered, or whether treatment is prophylactic or therapeutic. Usually, the patient is a human but non-human mammals including transgenic mammals can also be treated. Treatment dosages are optimally titrated to optimize safety and efficacy.

c. Immune Effector Cells and Methods of Engineering

In one aspect, provided herein are cells, e.g., immune effector cells, comprising a recombinant vector comprising a polycistronic expression cassette (e.g., a vector described herein). In some embodiments, the immune effector cell is a T cell. For example, in certain embodiments, the T cell is selected from the group consisting of a naïve T cell (CD4+ or CD8+); a killer CD8+ T cell; a CD4+ T cell corresponding to Th1, Th2, Th9, Th17, Th22, follicular helper (Tfh), regulatory (Treg) lineages; CD8⁺ cytotoxic T cell; a CD4⁺ cytotoxic T cell; a CD4⁺ helper T cell (e.g., a Th1 or a Th2 cell); a CD4/CD8 double positive T cell; a tumor infiltrating T cell (TIL); a thymocyte; a memory T cell, (e.g., a central memory T cell, effector memory T cell, a stem cell memory T cell or a stem cell memory-like T cell); and a natural killer T cell, e.g., an invariant natural killer T cell. In some embodiments, the T cell is a CD39^{neg}CD69^{neg} T cell or a CD8⁺CD39^{neg}CD69^{neg} cell, as described, e.g., in Krishna et al., “Stem-like CD8 T cells mediate response of adoptive cell immunotherapy against human cancer,” 2020 370(6522):1328-1334, which is incorporated by reference herein in its entirety. Precursor cells of the cellular immune system (e.g., precursors of T lymphocytes) are also useful for presenting a TCR disclosed herein because these cells may differentiate, develop, or mature into effector cells. Accordingly, in certain embodiments, the mammalian cell is a pluripotent stem cell (e.g., an embryonic stem cell, an induced pluripotent stem cell), a hematopoietic stem cell, or a lymphocyte progenitor cell. In certain embodiments, the hematopoietic stem cell or lymphocyte progenitor cell is isolated and/or enriched from, e.g., bone marrow, umbilical cord blood, or peripheral blood. In some embodiments, the immune effector cell is a CD4+ T cell. In some embodiments, the immune effector cell is a CD8+ T cell. In one aspect, provided herein is a population of immune effector cells comprising a polycistronic vector described herein. In some embodiments, the population of immune effector cells comprises CD4+ T cells and CD8+ T cells. In some embodiments, the population of immune effector cells are an ex vivo culture.

In another aspect, the present disclosure provides a population of engineered T cells, wherein most of the engineered T cells in the population comprise an exogenous TCR or functional fragment thereof, and wherein more than 2%, 3%, 4%, 5%, 6%, 7%, 8%, 9%, 10%, 15%, 20%, 25%, 30%, 35% or 40% of the population of engineered T cells are CCR7+/CD45RO+. In another aspect, the present disclosure provides a population of engineered T cells, wherein most of the engineered T cells in the population comprise an exogenous TCR or functional fragment thereof, and wherein more than 2%, 3%, 4%, 5%, 6%, 7%, 8%, 9%, 10%, 15%, 20%, 25%, 30%, 35% or 40% of the population of engineered T cells are memory T cells (e.g., a central memory T cell, an effector memory T cell, a stem cell-like memory T cells). In some embodiments, the T cells are electroporated with a vector that expresses the exogenous TCR or functional fragment thereof. In some embodiments, the T cells are cultured and/or expanded according to any of the methods provided herein.

In another aspect, the present disclosure provides a population of engineered T cells, wherein most of the engineered T cells in the population comprise an exogenous TCR or functional fragment thereof, and wherein more than 2%, 3%, 4%, 5%, 6%, 7%, 8%. 9%, 10%, 15%, 20%, 25%, 30%, 35% or 40% of the population of engineered T cells are CD95+/CD62L+. In another aspect, the present disclosure provides a population of engineered T cells, wherein most of the engineered T cells in the population comprise an exogenous TCR or functional fragment thereof, and wherein more than 2%, 3%, 4%, 5%, 6%, 7%, 8%, 9%, 10%, 15%, 20%, 25%, 30%, 35% or 40% of the population of engineered T cells are memory T cells (e.g., a central memory T cell, an effector memory T cell, a stem cell memory T cells). In some embodiments, the T cells are electroporated with a vector that expresses the exogenous TCR or functional fragment thereof. In some embodiments, the T cells are cultured and/or expanded according to any of the methods provided herein. In one aspect, provided herein are methods of introducing a vector described herein into a plurality of cells, e.g., immune effector cells, to produce a plurality of engineered cells, e.g., immune effector cells. Methods of introducing vectors into a cell are well known in the art. In the context of an expression vector, the vector can be readily introduced into a host cell, e.g., mammalian (e.g., human) cell by any method in the art. For example, the expression vector can be transferred into a host cell by transfection or transduction. Exemplary methods for introducing a vector into a host cell, include, but are not limited to, electroporation (also referred to herein as electro-transfer), sonication, calcium phosphate precipitation, lipofection, particle bombardment, microinjection, mechanical deformation by passage through a microfluidic device, and the like, see, e.g., Sambrook et al. Molecular Cloning: A Laboratory Manual, Cold Spring Harbor Laboratory, New York (2001), the entire contents of which is incorporated by reference herein. In some embodiments, a polycistronic vector is introduced into an immune effector cell or population of immune effector cells via electroporation. Alternative delivery systems include, e.g., colloidal dispersion systems, such as macromolecule complexes, nanocapsules, microspheres, beads, and lipid-based systems including oil-in-water emulsions, micelles, mixed micelles, and liposomes. In some embodiments, the polycistronic vector is introduced into a population of cells, e.g., immune effector cells, ex vivo, in vitro, or in vivo. In some embodiments, the polycistronic vector is introduced into a population of cells, e.g., immune effector cells, ex vivo.

i. Sources of Immune Effector Cells

Immune effector cells may be obtained from a subject by any suitable method known in the art. For example, T cells (e.g., CD4+ T cells and CD8+ T cells) can be obtained from several sources, including peripheral blood mononuclear cells, bone marrow, lymph node tissue, cord blood, thymus tissue, tissue from a site of infection, ascites, pleural effusion, spleen tissue, and tumors. In some embodiments, immune effector cells (e.g., T cells) are obtained from blood collected from a subject using any number of techniques known to the skilled artisan. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. T cells are isolated from peripheral blood lymphocytes by lysing the red blood cells and depleting the monocytes, for example, by centrifugation through a Percoll gradient or by counter flow centrifugal elutriation.

The cells collected by apheresis can be washed to remove the plasma fraction and to place the cells in an appropriate buffer (e.g., phosphate buffered saline (PBS)) or media for subsequent processing steps. The washing step may be accomplished by methods known to those in the art, such as by using a semi-automated “flow-through” centrifuge. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

A specific subpopulation of cells can be further isolated by positive or negative selection techniques (e.g., antibody coated beads, flow cytometry, etc.). In some embodiments, a specific subpopulation of T cells, such as CD3+, CD28+, CD4+, CD8+, CD45RA+, and CD45RO+ T cells, can be further isolated by positive or negative selection techniques (e.g., antibody coated beads, flow cytometry, etc.).

In certain embodiments, the mammalian cell is a population of cells presenting a TCR disclosed herein on the cell surface. The population of cells can be heterogeneous or homogenous. In certain embodiments, at least 50% (e.g., at least 60%, 70%, 80%, 90%, 95%, 99%, 99.5%, or 99.9%) of the population is a cell as described herein. In certain embodiments, the population is substantially pure, wherein at least 50% (e.g., at least 60%, 70%, 80%, 90%, 95%, 99%, 99.5%, or 99.9%) of the population is homogeneous. In certain embodiments, the population is heterogeneous and comprises a mixed population of cells (e.g., the cells have different cell types, developmental stages, origins, are isolated, purified, or enriched by different methods, are stimulated with different agents, and/or are engineered by different methods). In certain embodiments, the cells are a population of peripheral blood mononuclear cells (PBMC) (e.g., human PBMCs).

Populations of cells can be enriched or purified, as needed. In certain embodiments, regulatory T cells (e.g., CD25⁺ T cells) are depleted from the population, e.g., by using an anti-CD25 antibody conjugated to a surface such as a bead, particle, or cell. In certain embodiments, an anti-CD25 antibody is conjugated to a fluorescent dye (e.g., for use in fluorescence-activated cell sorting). In certain embodiments, cells expressing checkpoint receptors (e.g., CTLA-4, PD-1, TIM-3, LAG-3, TIGIT, VISTA, BTLA, TIGIT, CD137, or CEACAM1) are depleted from the population, e.g., by using an antibody that binds specifically to a checkpoint receptor conjugated to a surface such as a bead, particle, or cell. In certain embodiments, a T cell population can be selected so that it expresses one or more of IFN-γ, TNFα, IL-17A, IL-2, IL-3, IL-4, GM-CSF, IL-13, granzyme (e.g., granzyme B), and perforin, or other appropriate molecules, e.g., other cytokines. Methods for determining such expression are described, for example, in PCT Publication No.: WO 2013/126712, which is incorporated by reference herein in its entirety.

d. Pharmaceutical Compositions

Provided herein are pharmaceutical compositions comprising a population of engineered immune effector cells disclosed herein having the desired degree of purity in a physiologically acceptable carrier, excipient or stabilizer (see, e.g., Remington's Pharmaceutical Sciences (1990) Mack Publishing Co., Easton, PA). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Pharmaceutical compositions described herein can be useful in inducing an immune response in a subject and treating a condition, such as cancer. In one embodiment, the present disclosure provides a pharmaceutical composition comprising a population of engineered immune effector cells described herein for use as a medicament. In another embodiment, the disclosure provides a pharmaceutical composition for use in a method for the treatment of cancer. In some embodiments, pharmaceutical compositions comprise a population of engineered immune effector cells disclosed herein, and optionally one or more additional prophylactic or therapeutic agents, in a pharmaceutically acceptable carrier.

A pharmaceutical composition may be formulated for any route of administration to a subject. Specific examples of routes of administration include parenteral administration (e.g., intravenous, subcutaneous, intramuscular). In some embodiments, the pharmaceutical composition is formulated for intravenous administration. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

In some embodiments, the pharmaceutical composition is formulated for intravenous administration. Suitable carriers for intravenous administration include physiological saline or phosphate buffered saline (PBS), and solutions containing thickening and solubilizing agents, such as glucose, polyethylene glycol, and polypropylene glycol and mixtures thereof.

The compositions to be used for in vivo administration can be sterile. This is readily accomplished by filtration through, e.g., sterile filtration membranes.

Pharmaceutically acceptable carriers used in parenteral preparations include for example, aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include sodium chloride injection, Ringer's injection, isotonic dextrose injection, sterile water injection, dextrose and lactated Ringer's injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether treatment is prophylactic or therapeutic. Treatment dosages are optimally titrated to optimize safety and efficacy.

### 1.8 Kits

In one aspect, provided herein are kits comprising one or more pharmaceutical composition, population of engineered effector cells (e.g., recombinant cells), polynucleotide, or vector described herein and instructions for use. Such kits may include, e.g., a carrier, package, or container that is compartmentalized to receive one or more containers such as vials, tubes, and the like. Suitable containers include, for example, bottles, vials, syringes, and test tubes. In one embodiment, the containers are formed from a variety of materials such as glass or plastic.

In a specific embodiment, provided herein is a pharmaceutical kit comprising one or more containers filled with one or more of the ingredients of the pharmaceutical compositions described herein, population of engineered immune effector cells, polynucleotides, or vectors provided herein. In one embodiment, the kit comprises a pharmaceutical composition comprising a population of engineered immune effector cells described herein. In one embodiment, the kit comprises a pharmaceutical composition comprising a population of immune effector cells engineered according to a method described herein. In some embodiments, the kit contains a pharmaceutical composition described herein and a prophylactic or therapeutic agent. Optionally associated with such container(s) can be a notice in the form prescribed by a governmental agency regulating the manufacture, use or sale of pharmaceuticals or biological products, which notice reflects approval by the agency of manufacture, use or sale for human administration.

### 1.9 Non-Limiting Examples of Embodiments

Embodiment 1. A method of expanding a population of electroporated T cells comprising culturing the electroporated T cells with a first culture medium comprising an exogenous glutathione precursor and IL-15, wherein the electroporated T cells are contacted with the first culture medium within 12 hours of electroporation.

Embodiment 2. The method of embodiment 1, wherein the glutathione precursor is N-acetylcysteine (NAC).

Embodiment 3. The method of embodiment 1 or 2, wherein the first culture medium comprises IL-7.

Embodiment 4. The method of any one of embodiments 1 to 3, wherein the first culture medium comprises IL-21.

Embodiment 5. The method of any one of embodiments 1 to 4, wherein the first culture medium comprises IL-7 and IL-21.

Embodiment 6. The method of any one of embodiments 1 to 5, wherein the electroporated T cells are electroporated prior to culturing with the first culture medium.

Embodiment 7. The method of embodiment 6, wherein the electroporated T cells are cultured in the first culture medium for 6-12 hours after electroporation.

Embodiment 8. The method of any one of embodiments 1 to 7 further comprising culturing the T cells with a second culture medium, wherein the second culture medium comprises one or more cytokines selected from the group consisting of IL-7, IL-12, and IL-21.

Embodiment 9. The method of embodiment 8, wherein the second culture medium comprises IL-7, IL-12, and IL-21.

Embodiment 10. The method of embodiment 8 or 9, wherein IL-21 is added to the second culture medium every 2 to 3 days.

Embodiment 11. The method of any one of embodiments 8 to 10, wherein at least one of the cytokines selected from the group consisting of IL-7 and IL-12 are added to the second culture medium only on the first day of culturing.

Embodiment 12. The method of embodiment 11, wherein IL-7 and IL-12 are added to the second culture medium only on the first day of culturing.

Embodiment 13. The method of any one of embodiments 8 to 12, wherein the T cells are cultured in the second culture medium after being cultured in the first culture medium.

Embodiment 14. The method of any one of embodiments 8 to 13, wherein the T cells are cultured in the second culture medium for 11 to 13 days.

Embodiment 15. The method of any one of embodiments 1 to 14, further comprising culturing the T cells with a third culture medium, wherein the third culture medium comprises one or more cytokines selected from the group consisting of IL-2 and IL-21.

Embodiment 16. The method of embodiment 15, wherein the third culture medium comprises IL-2.

Embodiment 17. The method of embodiment 15 or 16, wherein the third culture medium comprises IL-21.

Embodiment 18. The method of any one of embodiments 15 to 17, wherein the third culture medium further comprises IL-12.

Embodiment 19. The method of any one of embodiments 15 to 18, wherein the third culture medium further comprises an exogenous glutathione precursor.

Embodiment 20. The method of embodiment 19, wherein the exogenous glutathione precursor in NAC.

Embodiment 21. The method of any one of embodiments 15 to 20, wherein the third culture medium comprises IL-12, IL-21 and NAC.

Embodiment 22. The method of any one of embodiments 15 to 21, wherein the third culture medium comprises IL-2, IL-12, IL-21 and NAC.

Embodiment 23. The method of any one of embodiments 17 to 22, wherein IL-21 is added to the third culture medium every 2 to 3 days.

Embodiment 24. The method of any one of embodiments 16 or 18 to 22, wherein IL-2 is added to the third culture medium every 3 to 4 days.

Embodiment 25. The method of any one of embodiments 16 or 18 to 23, wherein IL-2 is present in the third culture medium in an amount from 30 U/ml to 3000 U/ml.

Embodiment 26. The method of any one of embodiments 18 to 25, wherein the IL-12 is added to the third culture medium only on the first day of culturing.

Embodiment 27. The method of any one of embodiments 15 to 26, wherein the T cells are cultured in the third culture medium after being cultured in the second culture medium.

Embodiment 28. The method of any one of embodiments 15 to 27, wherein the T cells are cultured in the third culture medium for 11 to 13 days.

Embodiment 29. The method of any one of embodiments 1 to 28, wherein the first, second and/or third culture media further comprise a TCR agonist.

Embodiment 30. The method of embodiment 29, wherein the TCR agonist is a CD3 agonist.

Embodiment 31. The method of any one of embodiments 1 to 30, wherein the first, second and/or third culture media further comprise an agonist of a T cell costimulatory molecule.

Embodiment 32. The method of embodiment 31, wherein the agonist of a T cell costimulatory molecule is a CD28 agonist.

Embodiment 33. The method of any one of embodiments 1 to 32, wherein the first, second and/or third culture media further comprise a nanomatrix.

Embodiment 34. The method of embodiment 33, wherein the TCR agonist and/or the T cell costimulatory molecule is associated with the nanomatrix.

Embodiment 35. The method of any one of embodiments 1 to 34, further comprising culturing the cells with feeder cells.

Embodiment 36. A population of engineered T cells manufactured according to the method of any one of embodiments 1 to 35.

Embodiment 37. The population of engineered T cells of embodiment 36, wherein more than 10% of the engineered T cells in the population comprise one or more of the following: an exogenous TCR or functional fragment thereof, and an exogenous membrane-bound IL-15.

Embodiment 38. The population of engineered T cells of embodiment 36, wherein more than 2% of the engineered T cells in the population co-express an exogenous TCR or functional fragment thereof and an exogenous membrane-bound IL-15.

Embodiment 39. A population of engineered T cells, wherein more than 10% of the engineered T cells in the population comprise an exogenous TCR or functional fragment thereof, and wherein more than 20% of the population of engineered T cells are CCR7+/CD45RO+.

Embodiment 40. A population of engineered T cells, wherein more than 10% of the engineered T cells in the population comprise an exogenous TCR or functional fragment thereof, and wherein more than 40% of the population of engineered T cells are CD95+/CD62L+.

Embodiment 41. The population of engineered T cells of embodiment 40, wherein the population of engineered T cells comprise more than 5%, 10%, 15%, 20%, 25%, 30%, 35%, 40%, 45% or 50% CD45RA+CD45RO-CD62L+CD95+ cells.

Embodiment 42. The population of engineered T cells of embodiment 40, wherein the population of engineered T cells comprise more than 5%, 10%, 15%, 20%, 25%, 30%, 35%, 40%, 45% or 50% CD45RA+CD45RO+CD62L+CD95+ cells.

Embodiment 43. A population of cells comprising a polycistronic expression cassette comprising:
a. a first cistron comprising a polynucleotide sequence that encodes a fusion protein that comprises IL-15, or a functional fragment or functional variant thereof, and IL-15Rα, or a functional fragment or functional variant thereof,
b. a second cistron comprising a polynucleotide sequence that encodes a TCR beta chain comprising a Vβ region and a Cβ region; and
c. a third cistron comprising a polynucleotide sequence that encodes a TCR alpha chain comprising a Vα region and a Cα region, wherein the population of cells are T cells that comprise more than 5%, 10%, 15%, 20%, 25%, 30%, 35%, 40%, 45% or 50% CD45RA+CD45RO-CD62L+CD95+ cells.

Embodiment 44. A population of cells comprising a polycistronic expression cassette comprising:
d. a first cistron comprising a polynucleotide sequence that encodes a fusion protein that comprises IL-15, or a functional fragment or functional variant thereof, and IL-15Rα, or a functional fragment or functional variant thereof,
e. a second cistron comprising a polynucleotide sequence that encodes a TCR beta chain comprising a Vβ region and a Cβ region; and
f. a third cistron comprising a polynucleotide sequence that encodes a TCR alpha chain comprising a Vα region and a Cα region, wherein the population of cells are T cells that comprise more than 5%, 10%, 15%, 20%, 25%, 30%, 35%, 40%, 45% or 50% CD45RA+CD45RO+CD62L+CD95+ cells.

Embodiment 45. A method of expanding a population of electroporated T cells comprising culturing the electroporated T cells with a first culture medium comprising one or more cytokines.

Embodiment 46. The method of embodiment 45, wherein the electroporated T cells are contacted with the first culture medium within 12 hours of electroporation.

Embodiment 47. The method of embodiment 45 or 46, wherein the one or more cytokines are selected from the group consisting of IL-7, IL-15, and IL-21.

Embodiment 48. The method of any one of embodiments 45 to 47, wherein the first culture medium further comprises an exogenous glutathione precursor.

Embodiment 49. The method of embodiment 48, wherein the glutathione precursor is N-acetylcysteine (NAC).

Embodiment 50. The method of any one of embodiments 45 to 49, wherein the first culture medium comprises IL-15.

Embodiment 51. The method of any one of embodiments 45 to 50, wherein the first culture medium comprises IL-7.

Embodiment 52. The method of any one of embodiments 45 to 51, wherein the first culture medium comprises IL-21.

Embodiment 53. The method of any one of embodiments 45 to 52, wherein the first culture medium comprises IL-7 and IL-21.

Embodiment 54. The method of any one of embodiments 45 to 53, wherein the electroporated T cells were electroporated prior to culturing with the first culture medium.

Embodiment 55. The method of embodiment 54, wherein the electroporated T cells are cultured in the first culture medium for 6-12 hours after electroporation.

Embodiment 56. The method of any one of embodiments 45 to 55 further comprising culturing the T cells with a second culture medium, wherein the second culture medium comprises one or more cytokines selected from the group consisting of IL-7, IL-12, and IL-21.

Embodiment 57. The method of embodiment 56, wherein the second culture medium comprises IL-7, IL-12, and IL-21.

Embodiment 58. The method of embodiment 56 or 57, wherein IL-21 is added to the second culture medium every 2 to 3 days.

Embodiment 59. The method of any one of embodiments 56 to 58, wherein at least one of the cytokines selected from the group consisting of IL-7 and IL-12 are added to the second culture medium only on the first day of culturing.

Embodiment 60. The method of embodiment 59, wherein IL-7 and IL-12 are added to the second culture medium only on the first day of culturing.

Embodiment 61. The method of any one of embodiments 56 to 60, wherein the T cells are cultured in the second culture medium after being cultured in the first culture medium.

Embodiment 62. The method of any one of embodiments 56 to 61, wherein the T cells are cultured in the second culture medium for 11 to 13 days.

Embodiment 63. The method of any one of embodiments 45 to 62, further comprising culturing the T cells with a third culture medium, wherein the third culture medium comprises one or more cytokines selected from the group consisting of IL-2 and IL-21.

Embodiment 64. The method of embodiment 63, wherein the third culture medium comprises IL-2.

Embodiment 65. The method of embodiment 63 or 64, wherein the third culture medium comprises IL-21.

Embodiment 66. The method of any one of embodiments 63 to 65, wherein the third culture medium further comprises IL-12.

Embodiment 67. The method of any one of embodiments 63 to 66, wherein the third culture medium further comprises an exogenous glutathione precursor.

Embodiment 68. The method of embodiment 67, wherein the exogenous glutathione precursor is NAC.

Embodiment 69. The method of any one of embodiments 63 to 68, wherein the third culture medium comprises IL-12, IL-21 and NAC.

Embodiment 70. The method of any one of embodiments 63 to 69, wherein the third culture medium comprises IL-2, IL-12, IL-21 and NAC.

Embodiment 71. The method of any one of embodiments 65 to 70, wherein IL-21 is added to the third culture medium every 2 to 3 days.

Embodiment 72. The method of any one of embodiments 64 or 66 to 70, wherein IL-2 is added to the third culture medium every 3 to 4 days.

Embodiment 73. The method of any one of embodiments 64 or 66 to 71, wherein IL-2 is present in the third culture medium in an amount from 30 U/ml to 3000 U/ml.

Embodiment 74. The method of any one of embodiments 66 to 73, wherein the IL-12 is added to the third culture medium only on the first day of culturing.

Embodiment 75. The method of any one of embodiments 63 to 74, wherein the T cells are cultured in the third culture medium after being cultured in the second culture medium.

Embodiment 76. The method of any one of embodiments 63 to 75, wherein the T cells are cultured in the third culture medium for 11 to 13 days.

Embodiment 77. The method of any one of embodiments 45 to 76, wherein the first, second and/or third culture media further comprise a TCR agonist.

Embodiment 78. The method of embodiment 77, wherein the TCR agonist is a CD3 agonist.

Embodiment 79. The method of any one of embodiments 45 to 78, wherein the first, second and/or third culture media further comprise an agonist of a T cell costimulatory molecule.

Embodiment 80. The method of embodiment 79, wherein the agonist of a T cell costimulatory molecule is a CD28 agonist.

Embodiment 81. The method of any one of embodiments 45 to 80, wherein the first, second and/or third culture media further comprise a nanomatrix.

Embodiment 82. The method of embodiment 81, wherein the TCR agonist and/or the T cell costimulatory molecule is associated with the nanomatrix.

Embodiment 83. The method of any one of embodiments 45 to 82, further comprising culturing the cells with feeder cells.

Embodiment 84. A population of engineered T cells manufactured according to the method of any one of embodiments 45 to 83.

Embodiment 85. The population of engineered T cells of embodiment 84, wherein more than 10% of the engineered T cells in the population comprise one or more of the following: an exogenous TCR or functional fragment thereof, and an exogenous membrane-bound IL-15.

Embodiment 86. The population of engineered T cells of embodiment 84, wherein more than 2% of the engineered T cells in the population co-express an exogenous TCR or functional fragment thereof and an exogenous membrane-bound IL-15.

Embodiment 87. A population of engineered T cells, wherein more than 10% of the engineered T cells in the population comprise an exogenous TCR or functional fragment thereof, and wherein more than 20% of the population of engineered T cells are CCR7+/CD45RO+.

Embodiment 88. A population of engineered T cells, wherein more than 10% of the engineered T cells in the population comprise an exogenous TCR or functional fragment thereof, and wherein more than 40% of the population of engineered T cells are CD95+/CD62L+.

Embodiment 89. The population of engineered T cells of embodiment 88, wherein the population of engineered T cells comprise more than 5%, 10%, 15%, 20%, 25%, 30%, 35%, 40%, 45% or 50% CD45RA+CD45RO-CD62L+CD95+ cells.

Embodiment 90. The population of engineered T cells of embodiment 88, wherein the population of engineered T cells comprise more than 5%, 10%, 15%, 20%, 25%, 30%, 35%, 40%, 45% or 50% CD45RA+CD45RO+CD62L+CD95+ cells.

Embodiment 91. A population of cells comprising a polycistronic expression cassette comprising:
g. a first cistron comprising a polynucleotide sequence that encodes a fusion protein that comprises IL-15, or a functional fragment or functional variant thereof, and IL-15Rα, or a functional fragment or functional variant thereof,
h. a second cistron comprising a polynucleotide sequence that encodes a TCR beta chain comprising a Vβ region and a Cβ region; and
i. a third cistron comprising a polynucleotide sequence that encodes a TCR alpha chain comprising a Vα region and a Cα region.

Embodiment 92. The population of cells of embodiment 91, wherein the population of cells are T cells that comprise more than 5%, 10%, 15%, 20%, 25%, 30%, 35%, 40%, 45% or 50% CD45RA+CD45RO-CD62L+CD95+ cells.

Embodiment 93. The population of cells of embodiment 91, wherein the population of cells are T cells that comprise more than 5%, 10%, 15%, 20%, 25%, 30%, 35%, 40%, 45% or 50% CD45RA+CD45RO+CD62L+CD95+ cells.

### EXAMPLES

The examples of the present disclosure are offered by way of illustration and explanation, and are not intended to limit the scope of the present disclosure.

### Example 1: Construction of Transposon Plasmids

To improve homogeneity of multigene co-expression and product manufacturability, recombinant nucleic acid SB transposon plasmids comprising polycistronic expression cassettes were constructed. The polycistronic expression cassettes each include a transcriptional regulatory element operably linked to a polycistronic polynucleotide that encodes the TCR α chain of TCR001 (referred to herein as “TCRα” or “A”), the TCR β chain of TCR001 (referred to herein as “TCRβ” or “B”), and membrane-bound IL-15/IL-15Rα fusion protein (referred to herein as “mbIL15” or “15”), each separated by a furin recognition site and either a P2A element or a T2A element that mediates ribosome skipping to enable expression of separate polypeptide chains.

The TCR used in this Example, TCR001, is a chimeric TCR with murine-derived constant regions and with human Vα and Vβ regions specific for the R175H mutation of the p53 protein (in which position 175 of the p53 protein is mutated from Arg to His) in the context of HLA-A*02:01.

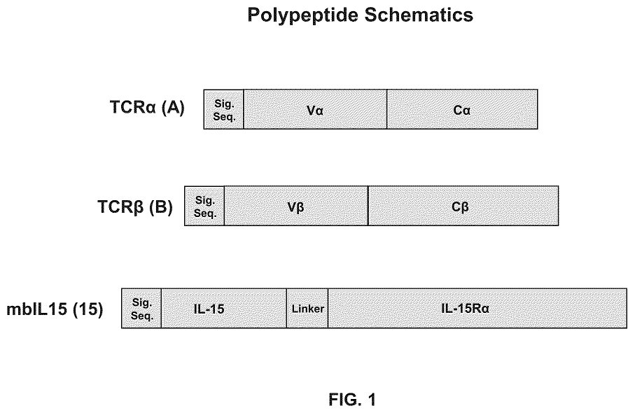

Briefly, TCRα was generated by fusing a human Vα region, including its N-terminal signal sequence (SEQ ID NO: 1006) with a glutamic acid at position 2, to a murine Cα region modified by substituting a cysteine at amino acid position 48, a leucine at amino acid position 112, an isoleucine at amino acid position 114, and a valine at amino acid position 115 (SEQ ID NO: 41). TCRβ was generated by fusing a human Vβ region, including its N-terminal signal sequence (SEQ ID NO: 2006) with an alanine at position 2, to a murine Cβ modified by substituting a cysteine at amino acid position 57 (SEQ ID NO: 51). mbIL15 was constructed by joining human IL-15 (SEQ ID NO: 76) to human IL-15Rα (SEQ ID NO: 78) via a Gly-Ser-rich linker peptide (SEQ ID NO: 81), with an IgE signal sequence (SEQ ID NO: 83) N-terminal to the human IL-15. Schematics of each of these three polypeptide constructs are shown in FIG. 1, from N terminus (left) to C terminus (right) for each construct.

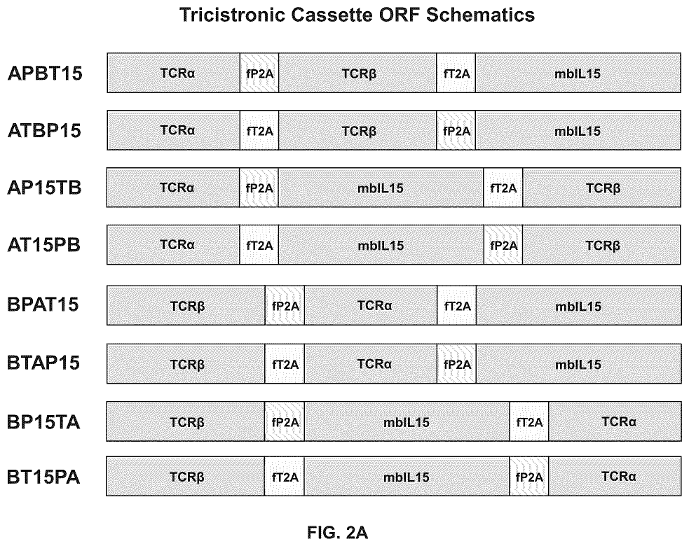

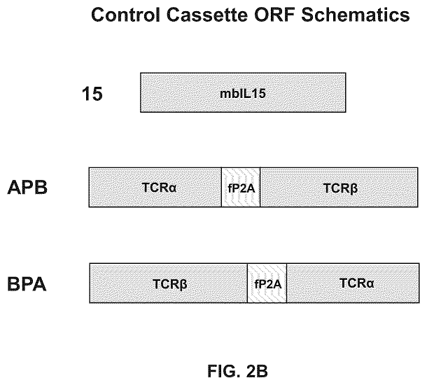

To explore the effect of gene/element order on expression and function, eight tricistronic polynucleotide expression cassettes were generated with polynucleotides encoding each of TCRα, TCRβ, and mbIL15. In each expression cassette, these three elements were fused pairwise with a) a polynucleotide encoding a furin recognition site joined to a P2A element (SEQ ID NO: 11) (referred to herein as “fP2A” or “P”) and b) a polynucleotide encoding a furin recognition site joined to a T2A element (SEQ ID NO: 13) (referred to herein as “fT2A” or “T”). The resulting tricistronic expression cassettes, including suitable transcriptional regulatory elements, were inserted between the ITRs of Sleeping Beauty (SB) transposon plasmids. The 5′ to 3′ order of elements in the open reading frame (ORF) of each expression cassette and SB Plasmid is shown in Table E1, and schematics of the ORFs of these eight expression cassettes are shown in FIG. 2A.

TABLE E1
Tricistronic SB transposon plasmids.

| Plasmid Name | Cassette Name | Order of Elements (5′ to 3′) |
|---|---|---|
| Plasmid APBT15 | Cassette APBT15 | TCRα-fP2A-TCRβ-fT2A-mbIL15 |
| Plasmid ATBP15 | Cassette ATBP15 | TCRα-fT2A-TCRβ-fP2A-mbIL15 |
| Plasmid AP15TB | Cassette AP15TB | TCRα-fP2A- mbIL15-fT2A-TCRβ |
| Plasmid AT15PB | Cassette AT15PB | TCRα-fT2A- mbIL15-fP2A-TCRβ |
| Plasmid BPAT15 | Cassette BPAT15 | TCRβ-fP2A-TCRα-fT2A-mbIL15 |
| Plasmid BTAP15 | Cassette BTAP15 | TCRβ-fT2A-TCRα-fP2A-mbIL15 |
| Plasmid BP15TA | Cassette BP15TA | TCRβ-fP2A- mbIL15-fT2A-TCRα |
| Plasmid BT15PA | Cassette BT15PA | TCRβ-fT2A- mbIL15-fP2A-TCRα |

The polynucleotide sequences of the ORFs of these transposon plasmids is shown in Table E2.

TABLE E2
Polynucleotide sequences of SB plasmid ORFs.

| | | SEQ |
|---|---|---|
| | | ID |
| Plasmid | Polynucleotide Sequence of ORF | NO: |
| Plasmid | ATGGAGTCCTTTCTGGGCGGCGTGCTGCTGATCCTGTGGCTGCAGGTGGACTGGGTGAAAT | 320 |
| APBT15 | CCCAGAAGATCGAGCAGAACTCTGAGGCGCTGAATATTCAGGAGGGCAAGACCGCGACACT | |
| | GACCTGCAACTACACAAATTATTCCCCAGCGTACCTGCAGTGGTATAGGCAGGACCCAGGC | |
| | AGGGGACCCGTGTTTCTGCTGCTGATTCGGGAGAATGAGAAGGAGAAAAGAAAGGAGAGGC | |
| | TGAAAGTGACCTTCGATACCACACTGAAGCAGTCTCTGTTTCACATCACAGCGTCTCAGCC | |
| | AGCGGACAGCGCGACCTACCTGTGCGCGCTGGACATCTACCCTCACGATATGAGATTCGGC | |
| | GCGGGCACAAGGCTGACCGTGAAACCAAACATCCAGAATCCCGAGCCTGCGGTGTACCAGC | |
| | TGAAGGACCCCCGCTCTCAGGATAGCACACTGTGCCTGTTCACCGACTTTGATAGCCAGAT | |
| | CAACGTGCCTAAAACAATGGAGTCCGGCACCTTCATCACCGACAAGTGCGTGCTGGATATG | |
| | AAAGCGATGGACTCCAAGTCTAACGGCGCGATCGCGTGGTCCAATCAGACATCTTTCACCT | |
| | GCCAGGATATCTTCAAGGAGACAAACGCGACCTATCCTTCCTCTGACGTGCCATGTGATGC | |
| | GACACTGACCGAGAAGAGCTTCGAGACAGACATGAACCTGAATTTTCAGAATCTGCTGGTC | |
| | ATCGTGCTGAGAATCCTGCTGCTGAAGGTGGCGGGCTTTAATCTGCTGATGACACTGCGGC | |
| | TGTGGAGTTCCCGGGCGAAACGCTCTGGAAGCGGAGCGACCAATTTCAGCCTGCTGAAGCA | |
| | GGCGGGCGATGTGGAGGAGAACCCTGGCCCAATGGCGACAAGACTGCTGTGCTGGGCGGCG | |
| | CTGTGCCTGCTGGGAGCGGAACTGACTGAAGCGGGGGTCGCGCAGAGCCCTCGATACAAAA | |
| | TCATTGAGAAGCGGCAGTCTGTGGCGTTCTGGTGCAACCCAATCAGCGGACACGCGACCCT | |
| | GTACTGGTATCAGCAGATCCTGGGCCAGGGCCCTAAGCTGCTGATTCAGTTCCAGAACAAT | |
| | GGCGTGGTGGACGATAGCCAGCTGCCAAAAGATAGATTTTCCGCGGAGAGGCTGAAGGGCG | |
| | TGGACTCTACACTGAAAATTCAGCCTGCGAAGCTGGAGGATAGCGCGGTGTACCTGTGCGC | |
| | GAGCTCCCTGGACCCAGGCGATACCGGAGAGCTGTTCTTTGGAGAGGGCAGCCGGCTGACA | |
| | GTGCTGGAGGACCTGAGGAACGTGACCCCACCTAAAGTGAGCCTGTTCGAGCCATCCAAGG | |
| | CGGAGATCGCGAATAAGCAGAAAGCGACCCTGGTGTGCCTGGCGAGGGGCTTCTTTCCCGA | |
| | TCACGTGGAGCTGTCCTGGTGGGTGAACGGCAAAGAGGTGCACTCTGGCGTGTGCACAGAC | |
| | CCTCAGGCGTACAAGGAGAGCAATTACTCCTATTGTCTGTCTAGCAGACTGAGGGTGAGCG | |
| | CGACCTTTTGGCACAACCCCCGGAATCACTTCCGCTGCCAGGTGCAGTTTCACGGCCTGTC | |
| | CGAGGAGGATAAATGGCCTGAGGGCTCTCCAAAGCCCGTGACACAGAATATCAGCGCGGAG | |
| | GCGTGGGGAAGAGCGGACTGTGGCATTACAAGCGCGTCCTATCAGCAGGGCGTGCTGTCCG | |
| | CGACCATCCTGTACGAGATTCTGCTGGGCAAGGCGACACTGTATGCGGTGCTGGTGTCCAC | |
| | CCTGGTGGTCATGGCGATGGTGAAGAGGAAAAACTCTCGGGCGAAACGCTCTGGAAGCGGA | |
| | GAGGGCAGAGGAAGTCTTCTAACATGCGGTGACGTGGAGGAGAATCCCGGCCCTATGGATT | |
| | GGACCTGGATTCTGTTTCTGGTGGCCGCTGCCACAAGAGTGCACAGCAACTGGGTGAATGT | |
| | GATCAGCGACCTGAAGAAGATCGAGGATCTGATCCAGAGCATGCACATTGATGCCACCCTG | |
| | TACACAGAATCTGATGTGCACCCTAGCTGTAAAGTGACCGCCATGAAGTGTTTTCTGCTGG | |
| | AGCTGCAGGTGATTTCTCTGGAAAGCGGAGATGCCTCTATCCACGACACAGTGGAGAATCT | |
| | GATCATCCTGGCCAACAATAGCCTGAGCAGCAATGGCAATGTGACAGAGTCTGGCTGTAAG | |
| | GAGTGTGAGGAGCTGGAGGAGAAGAACATCAAGGAGTTTCTGCAGAGCTTTGTGCACATCG | |
| | TGCAGATGTTCATCAATACAAGCTCTGGCGGAGGATCTGGAGGAGGCGGATCTGGAGGAGG | |
| | AGGCAGTGGAGGCGGAGGATCTGGCGGAGGATCTCTGCAGATTACATGCCCTCCTCCAATG | |
| | TCTGTGGAGCACGCCGATATTTGGGTGAAGTCCTACAGCCTGTACAGCAGAGAGAGATACA | |
| | TCTGCAACAGCGGCTTTAAGAGAAAGGCCGGCACCTCTTCTCTGACAGAGTGCGTGCTGAA | |
| | TAAGGCCACAAATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTGACACCTCAGC | |
| | CTGAATCTCTGAGCCCTTCTGGAAAAGAACCTGCCGCCAGCTCTCCTAGCTCTAATAATAC | |
| | CGCCGCCACAACAGCCGCCATTGTGCCTGGATCTCAGCTGATGCCTAGCAAGTCTCCTAGC | |
| | ACAGGCACAACAGAGATCAGCAGCCACGAATCTTCTCACGGAACACCTTCTCAGACCACCG | |
| | CCAAGAATTGGGAGCTGACAGCCTCTGCCTCTCACCAGCCTCCAGGAGTGTATCCTCAGGG | |
| | CCACTCTGATACAACAGTGGCCATCAGCACATCTACAGTGCTGCTGTGTGGACTGTCTGCC | |
| | GTGTCTCTGCTGGCCTGTTACCTGAAGTCTAGACAGACACCTCCTCTGGCCTCTGTGGAGA | |
| | TGGAGGCCATGGAAGCCCTGCCTGTGACATGGGGAACAAGCAGCAGAGATGAAGACCTGGA | |
| | GAATTGTTCTCACCACCTG | |
| Plasmid | ATGGAGTCCTTTCTGGGCGGCGTGCTGCTGATCCTGTGGCTGCAGGTGGACTGGGTGAAAT | 321 |
| ATBP15 | CCCAGAAGATCGAGCAGAACTCTGAGGCGCTGAATATTCAGGAGGGCAAGACCGCGACACT | |
| | GACCTGCAACTACACAAATTATTCCCCAGCGTACCTGCAGTGGTATAGGCAGGACCCAGGC | |
| | AGGGGACCCGTGTTTCTGCTGCTGATTCGGGAGAATGAGAAGGAGAAAAGAAAGGAGAGGC | |
| | TGAAAGTGACCTTCGATACCACACTGAAGCAGTCTCTGTTTCACATCACAGCGTCTCAGCC | |
| | AGCGGACAGCGCGACCTACCTGTGCGCGCTGGACATCTACCCTCACGATATGAGATTCGGC | |
| | GCGGGCACAAGGCTGACCGTGAAACCAAACATCCAGAATCCCGAGCCTGCGGTGTACCAGC | |
| | TGAAGGACCCCCGCTCTCAGGATAGCACACTGTGCCTGTTCACCGACTTTGATAGCCAGAT | |
| | CAACGTGCCTAAAACAATGGAGTCCGGCACCTTCATCACCGACAAGTGCGTGCTGGATATG | |
| | AAAGCGATGGACTCCAAGTCTAACGGCGCGATCGCGTGGTCCAATCAGACATCTTTCACCT | |
| | GCCAGGATATCTTCAAGGAGACAAACGCGACCTATCCTTCCTCTGACGTGCCATGTGATGC | |
| | GACACTGACCGAGAAGAGCTTCGAGACAGACATGAACCTGAATTTTCAGAATCTGCTGGTC | |
| | ATCGTGCTGAGAATCCTGCTGCTGAAGGTGGCGGGCTTTAATCTGCTGATGACACTGCGGC | |
| | TGTGGAGTTCCCGGGCGAAACGCTCTGGAAGCGGAGAGGGCAGAGGAAGTCTTCTAACATG | |
| | CGGTGACGTGGAGGAGAATCCCGGCCCTATGGCGACAAGACTGCTGTGCTGGGCGGCGCTG | |
| | TGCCTGCTGGGAGCGGAACTGACTGAAGCGGGGGTCGCGCAGAGCCCTCGATACAAAATCA | |
| | TTGAGAAGCGGCAGTCTGTGGCGTTCTGGTGCAACCCAATCAGCGGACACGCGACCCTGTA | |
| | CTGGTATCAGCAGATCCTGGGCCAGGGCCCTAAGCTGCTGATTCAGTTCCAGAACAATGGC | |
| | GTGGTGGACGATAGCCAGCTGCCAAAAGATAGATTTTCCGCGGAGAGGCTGAAGGGCGTGG | |
| | ACTCTACACTGAAAATTCAGCCTGCGAAGCTGGAGGATAGCGCGGTGTACCTGTGCGCGAG | |
| | CTCCCTGGACCCAGGCGATACCGGAGAGCTGTTCTTTGGAGAGGGCAGCCGGCTGACAGTG | |
| | CTGGAGGACCTGAGGAACGTGACCCCACCTAAAGTGAGCCTGTTCGAGCCATCCAAGGCGG | |
| | AGATCGCGAATAAGCAGAAAGCGACCCTGGTGTGCCTGGCGAGGGGCTTCTTTCCCGATCA | |
| | CGTGGAGCTGTCCTGGTGGGTGAACGGCAAAGAGGTGCACTCTGGCGTGTGCACAGACCCT | |
| | CAGGCGTACAAGGAGAGCAATTACTCCTATTGTCTGTCTAGCAGACTGAGGGTGAGCGCGA | |
| | CCTTTTGGCACAACCCCCGGAATCACTTCCGCTGCCAGGTGCAGTTTCACGGCCTGTCCGA | |
| | GGAGGATAAATGGCCTGAGGGCTCTCCAAAGCCCGTGACACAGAATATCAGCGCGGAGGCG | |
| | TGGGGAAGAGCGGACTGTGGCATTACAAGCGCGTCCTATCAGCAGGGCGTGCTGTCCGCGA | |
| | CCATCCTGTACGAGATTCTGCTGGGCAAGGCGACACTGTATGCGGTGCTGGTGTCCACCCT | |
| | GGTGGTCATGGCGATGGTGAAGAGGAAAAACTCTCGGGCGAAACGCTCTGGAAGCGGAGCG | |
| | ACCAATTTCAGCCTGCTGAAGCAGGCGGGCGATGTGGAGGAGAACCCTGGCCCAATGGATT | |
| | GGACCTGGATTCTGTTTCTGGTGGCCGCTGCCACAAGAGTGCACAGCAACTGGGTGAATGT | |
| | GATCAGCGACCTGAAGAAGATCGAGGATCTGATCCAGAGCATGCACATTGATGCCACCCTG | |
| | TACACAGAATCTGATGTGCACCCTAGCTGTAAAGTGACCGCCATGAAGTGTTTTCTGCTGG | |
| | AGCTGCAGGTGATTTCTCTGGAAAGCGGAGATGCCTCTATCCACGACACAGTGGAGAATCT | |
| | GATCATCCTGGCCAACAATAGCCTGAGCAGCAATGGCAATGTGACAGAGTCTGGCTGTAAG | |
| | GAGTGTGAGGAGCTGGAGGAGAAGAACATCAAGGAGTTTCTGCAGAGCTTTGTGCACATCG | |
| | TGCAGATGTTCATCAATACAAGCTCTGGCGGAGGATCTGGAGGAGGCGGATCTGGAGGAGG | |
| | AGGCAGTGGAGGCGGAGGATCTGGCGGAGGATCTCTGCAGATTACATGCCCTCCTCCAATG | |
| | TCTGTGGAGCACGCCGATATTTGGGTGAAGTCCTACAGCCTGTACAGCAGAGAGAGATACA | |
| | TCTGCAACAGCGGCTTTAAGAGAAAGGCCGGCACCTCTTCTCTGACAGAGTGCGTGCTGAA | |
| | TAAGGCCACAAATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTGACACCTCAGC | |
| | CTGAATCTCTGAGCCCTTCTGGAAAAGAACCTGCCGCCAGCTCTCCTAGCTCTAATAATAC | |
| | CGCCGCCACAACAGCCGCCATTGTGCCTGGATCTCAGCTGATGCCTAGCAAGTCTCCTAGC | |
| | ACAGGCACAACAGAGATCAGCAGCCACGAATCTTCTCACGGAACACCTTCTCAGACCACCG | |
| | CCAAGAATTGGGAGCTGACAGCCTCTGCCTCTCACCAGCCTCCAGGAGTGTATCCTCAGGG | |
| | CCACTCTGATACAACAGTGGCCATCAGCACATCTACAGTGCTGCTGTGTGGACTGTCTGCC | |
| | GTGTCTCTGCTGGCCTGTTACCTGAAGTCTAGACAGACACCTCCTCTGGCCTCTGTGGAGA | |
| | TGGAGGCCATGGAAGCCCTGCCTGTGACATGGGGAACAAGCAGCAGAGATGAAGACCTGGA | |
| | GAATTGTTCTCACCACCTG | |
| Plasmid | ATGGAGTCCTTTCTGGGCGGCGTGCTGCTGATCCTGTGGCTGCAGGTGGACTGGGTGAAAT | 322 |
| AP15TB | CCCAGAAGATCGAGCAGAACTCTGAGGCGCTGAATATTCAGGAGGGCAAGACCGCGACACT | |
| | GACCTGCAACTACACAAATTATTCCCCAGCGTACCTGCAGTGGTATAGGCAGGACCCAGGC | |
| | AGGGGACCCGTGTTTCTGCTGCTGATTCGGGAGAATGAGAAGGAGAAAAGAAAGGAGAGGC | |
| | TGAAAGTGACCTTCGATACCACACTGAAGCAGTCTCTGTTTCACATCACAGCGTCTCAGCC | |
| | AGCGGACAGCGCGACCTACCTGTGCGCGCTGGACATCTACCCTCACGATATGAGATTCGGC | |
| | GCGGGCACAAGGCTGACCGTGAAACCAAACATCCAGAATCCCGAGCCTGCGGTGTACCAGC | |
| | TGAAGGACCCCCGCTCTCAGGATAGCACACTGTGCCTGTTCACCGACTTTGATAGCCAGAT | |
| | CAACGTGCCTAAAACAATGGAGTCCGGCACCTTCATCACCGACAAGTGCGTGCTGGATATG | |
| | AAAGCGATGGACTCCAAGTCTAACGGCGCGATCGCGTGGTCCAATCAGACATCTTTCACCT | |
| | GCCAGGATATCTTCAAGGAGACAAACGCGACCTATCCTTCCTCTGACGTGCCATGTGATGC | |
| | GACACTGACCGAGAAGAGCTTCGAGACAGACATGAACCTGAATTTTCAGAATCTGCTGGTC | |
| | ATCGTGCTGAGAATCCTGCTGCTGAAGGTGGCGGGCTTTAATCTGCTGATGACACTGCGGC | |
| | TGTGGAGTTCCCGGGCGAAACGCTCTGGAAGCGGAGCGACCAATTTCAGCCTGCTGAAGCA | |
| | GGCGGGCGATGTGGAGGAGAACCCTGGCCCAATGGATTGGACCTGGATTCTGTTTCTGGTG | |
| | GCCGCTGCCACAAGAGTGCACAGCAACTGGGTGAATGTGATCAGCGACCTGAAGAAGATCG | |
| | AGGATCTGATCCAGAGCATGCACATTGATGCCACCCTGTACACAGAATCTGATGTGCACCC | |
| | TAGCTGTAAAGTGACCGCCATGAAGTGTTTTCTGCTGGAGCTGCAGGTGATTTCTCTGGAA | |
| | AGCGGAGATGCCTCTATCCACGACACAGTGGAGAATCTGATCATCCTGGCCAACAATAGCC | |
| | TGAGCAGCAATGGCAATGTGACAGAGTCTGGCTGTAAGGAGTGTGAGGAGCTGGAGGAGAA | |
| | GAACATCAAGGAGTTTCTGCAGAGCTTTGTGCACATCGTGCAGATGTTCATCAATACAAGC | |
| | TCTGGCGGAGGATCTGGAGGAGGCGGATCTGGAGGAGGAGGCAGTGGAGGCGGAGGATCTG | |
| | GCGGAGGATCTCTGCAGATTACATGCCCTCCTCCAATGTCTGTGGAGCACGCCGATATTTG | |
| | GGTGAAGTCCTACAGCCTGTACAGCAGAGAGAGATACATCTGCAACAGCGGCTTTAAGAGA | |
| | AAGGCCGGCACCTCTTCTCTGACAGAGTGCGTGCTGAATAAGGCCACAAATGTGGCCCACT | |
| | GGACAACACCTAGCCTGAAGTGCATTAGAGATCCTGCCCTGGTCCACCAGAGGCCTGCCCC | |
| | TCCATCTACAGTGACAACAGCCGGAGTGACACCTCAGCCTGAATCTCTGAGCCCTTCTGGA | |
| | AAAGAACCTGCCGCCAGCTCTCCTAGCTCTAATAATACCGCCGCCACAACAGCCGCCATTG | |
| | TGCCTGGATCTCAGCTGATGCCTAGCAAGTCTCCTAGCACAGGCACAACAGAGATCAGCAG | |
| | CCACGAATCTTCTCACGGAACACCTTCTCAGACCACCGCCAAGAATTGGGAGCTGACAGCC | |
| | TCTGCCTCTCACCAGCCTCCAGGAGTGTATCCTCAGGGCCACTCTGATACAACAGTGGCCA | |
| | TCAGCACATCTACAGTGCTGCTGTGTGGACTGTCTGCCGTGTCTCTGCTGGCCTGTTACCT | |
| | GAAGTCTAGACAGACACCTCCTCTGGCCTCTGTGGAGATGGAGGCCATGGAAGCCCTGCCT | |
| | GTGACATGGGGAACAAGCAGCAGAGATGAAGACCTGGAGAATTGTTCTCACCACCTGCGGG | |
| | CGAAACGCTCTGGAAGCGGAGAGGGCAGAGGAAGTCTTCTAACATGCGGTGACGTGGAGGA | |
| | GAATCCCGGCCCTATGGCGACAAGACTGCTGTGCTGGGCGGCGCTGTGCCTGCTGGGAGCG | |
| | GAACTGACTGAAGCGGGGGTCGCGCAGAGCCCTCGATACAAAATCATTGAGAAGCGGCAGT | |
| | CTGTGGCGTTCTGGTGCAACCCAATCAGCGGACACGCGACCCTGTACTGGTATCAGCAGAT | |
| | CCTGGGCCAGGGCCCTAAGCTGCTGATTCAGTTCCAGAACAATGGCGTGGTGGACGATAGC | |
| | CAGCTGCCAAAAGATAGATTTTCCGCGGAGAGGCTGAAGGGCGTGGACTCTACACTGAAAA | |
| | TTCAGCCTGCGAAGCTGGAGGATAGCGCGGTGTACCTGTGCGCGAGCTCCCTGGACCCAGG | |
| | CGATACCGGAGAGCTGTTCTTTGGAGAGGGCAGCCGGCTGACAGTGCTGGAGGACCTGAGG | |
| | AACGTGACCCCACCTAAAGTGAGCCTGTTCGAGCCATCCAAGGCGGAGATCGCGAATAAGC | |
| | AGAAAGCGACCCTGGTGTGCCTGGCGAGGGGCTTCTTTCCCGATCACGTGGAGCTGTCCTG | |
| | GTGGGTGAACGGCAAAGAGGTGCACTCTGGCGTGTGCACAGACCCTCAGGCGTACAAGGAG | |
| | AGCAATTACTCCTATTGTCTGTCTAGCAGACTGAGGGTGAGCGCGACCTTTTGGCACAACC | |
| | CCCGGAATCACTTCCGCTGCCAGGTGCAGTTTCACGGCCTGTCCGAGGAGGATAAATGGCC | |
| | TGAGGGCTCTCCAAAGCCCGTGACACAGAATATCAGCGCGGAGGCGTGGGGAAGAGCGGAC | |
| | TGTGGCATTACAAGCGCGTCCTATCAGCAGGGCGTGCTGTCCGCGACCATCCTGTACGAGA | |
| | TTCTGCTGGGCAAGGCGACACTGTATGCGGTGCTGGTGTCCACCCTGGTGGTCATGGCGAT | |
| | GGTGAAGAGGAAAAACTCT | |
| Plasmid | ATGGAGTCCTTTCTGGGCGGCGTGCTGCTGATCCTGTGGCTGCAGGTGGACTGGGTGAAAT | 323 |
| AT15PB | CCCAGAAGATCGAGCAGAACTCTGAGGCGCTGAATATTCAGGAGGGCAAGACCGCGACACT | |
| | GACCTGCAACTACACAAATTATTCCCCAGCGTACCTGCAGTGGTATAGGCAGGACCCAGGC | |
| | AGGGGACCCGTGTTTCTGCTGCTGATTCGGGAGAATGAGAAGGAGAAAAGAAAGGAGAGGC | |
| | TGAAAGTGACCTTCGATACCACACTGAAGCAGTCTCTGTTTCACATCACAGCGTCTCAGCC | |
| | AGCGGACAGCGCGACCTACCTGTGCGCGCTGGACATCTACCCTCACGATATGAGATTCGGC | |
| | GCGGGCACAAGGCTGACCGTGAAACCAAACATCCAGAATCCCGAGCCTGCGGTGTACCAGC | |
| | TGAAGGACCCCCGCTCTCAGGATAGCACACTGTGCCTGTTCACCGACTTTGATAGCCAGAT | |
| | CAACGTGCCTAAAACAATGGAGTCCGGCACCTTCATCACCGACAAGTGCGTGCTGGATATG | |
| | AAAGCGATGGACTCCAAGTCTAACGGCGCGATCGCGTGGTCCAATCAGACATCTTTCACCT | |
| | GCCAGGATATCTTCAAGGAGACAAACGCGACCTATCCTTCCTCTGACGTGCCATGTGATGC | |
| | GACACTGACCGAGAAGAGCTTCGAGACAGACATGAACCTGAATTTTCAGAATCTGCTGGTC | |
| | ATCGTGCTGAGAATCCTGCTGCTGAAGGTGGCGGGCTTTAATCTGCTGATGACACTGCGGC | |
| | TGTGGAGTTCCCGGGCGAAACGCTCTGGAAGCGGAGAGGGCAGAGGAAGTCTTCTAACATG | |
| | CGGTGACGTGGAGGAGAATCCCGGCCCTATGGATTGGACCTGGATTCTGTTTCTGGTGGCC | |
| | GCTGCCACAAGAGTGCACAGCAACTGGGTGAATGTGATCAGCGACCTGAAGAAGATCGAGG | |
| | ATCTGATCCAGAGCATGCACATTGATGCCACCCTGTACACAGAATCTGATGTGCACCCTAG | |
| | CTGTAAAGTGACCGCCATGAAGTGTTTTCTGCTGGAGCTGCAGGTGATTTCTCTGGAAAGC | |
| | GGAGATGCCTCTATCCACGACACAGTGGAGAATCTGATCATCCTGGCCAACAATAGCCTGA | |
| | GCAGCAATGGCAATGTGACAGAGTCTGGCTGTAAGGAGTGTGAGGAGCTGGAGGAGAAGAA | |
| | CATCAAGGAGTTTCTGCAGAGCTTTGTGCACATCGTGCAGATGTTCATCAATACAAGCTCT | |
| | GGCGGAGGATCTGGAGGAGGCGGATCTGGAGGAGGAGGCAGTGGAGGCGGAGGATCTGGCG | |
| | GAGGATCTCTGCAGATTACATGCCCTCCTCCAATGTCTGTGGAGCACGCCGATATTTGGGT | |
| | GAAGTCCTACAGCCTGTACAGCAGAGAGAGATACATCTGCAACAGCGGCTTTAAGAGAAAG | |
| | GCCGGCACCTCTTCTCTGACAGAGTGCGTGCTGAATAAGGCCACAAATGTGGCCCACTGGA | |
| | CAACACCTAGCCTGAAGTGCATTAGAGATCCTGCCCTGGTCCACCAGAGGCCTGCCCCTCC | |
| | ATCTACAGTGACAACAGCCGGAGTGACACCTCAGCCTGAATCTCTGAGCCCTTCTGGAAAA | |
| | GAACCTGCCGCCAGCTCTCCTAGCTCTAATAATACCGCCGCCACAACAGCCGCCATTGTGC | |
| | CTGGATCTCAGCTGATGCCTAGCAAGTCTCCTAGCACAGGCACAACAGAGATCAGCAGCCA | |
| | CGAATCTTCTCACGGAACACCTTCTCAGACCACCGCCAAGAATTGGGAGCTGACAGCCTCT | |
| | GCCTCTCACCAGCCTCCAGGAGTGTATCCTCAGGGCCACTCTGATACAACAGTGGCCATCA | |
| | GCACATCTACAGTGCTGCTGTGTGGACTGTCTGCCGTGTCTCTGCTGGCCTGTTACCTGAA | |
| | GTCTAGACAGACACCTCCTCTGGCCTCTGTGGAGATGGAGGCCATGGAAGCCCTGCCTGTG | |
| | ACATGGGGAACAAGCAGCAGAGATGAAGACCTGGAGAATTGTTCTCACCACCTGCGGGCGA | |
| | AACGCTCTGGAAGCGGAGCGACCAATTTCAGCCTGCTGAAGCAGGCGGGCGATGTGGAGGA | |
| | GAACCCTGGCCCAATGGCGACAAGACTGCTGTGCTGGGCGGCGCTGTGCCTGCTGGGAGCG | |
| | GAACTGACTGAAGCGGGGGTCGCGCAGAGCCCTCGATACAAAATCATTGAGAAGCGGCAGT | |
| | CTGTGGCGTTCTGGTGCAACCCAATCAGCGGACACGCGACCCTGTACTGGTATCAGCAGAT | |
| | CCTGGGCCAGGGCCCTAAGCTGCTGATTCAGTTCCAGAACAATGGCGTGGTGGACGATAGC | |
| | CAGCTGCCAAAAGATAGATTTTCCGCGGAGAGGCTGAAGGGCGTGGACTCTACACTGAAAA | |
| | TTCAGCCTGCGAAGCTGGAGGATAGCGCGGTGTACCTGTGCGCGAGCTCCCTGGACCCAGG | |
| | CGATACCGGAGAGCTGTTCTTTGGAGAGGGCAGCCGGCTGACAGTGCTGGAGGACCTGAGG | |
| | AACGTGACCCCACCTAAAGTGAGCCTGTTCGAGCCATCCAAGGCGGAGATCGCGAATAAGC | |
| | AGAAAGCGACCCTGGTGTGCCTGGCGAGGGGCTTCTTTCCCGATCACGTGGAGCTGTCCTG | |
| | GTGGGTGAACGGCAAAGAGGTGCACTCTGGCGTGTGCACAGACCCTCAGGCGTACAAGGAG | |
| | AGCAATTACTCCTATTGTCTGTCTAGCAGACTGAGGGTGAGCGCGACCTTTTGGCACAACC | |
| | CCCGGAATCACTTCCGCTGCCAGGTGCAGTTTCACGGCCTGTCCGAGGAGGATAAATGGCC | |
| | TGAGGGCTCTCCAAAGCCCGTGACACAGAATATCAGCGCGGAGGCGTGGGGAAGAGCGGAC | |
| | TGTGGCATTACAAGCGCGTCCTATCAGCAGGGCGTGCTGTCCGCGACCATCCTGTACGAGA | |
| | TTCTGCTGGGCAAGGCGACACTGTATGCGGTGCTGGTGTCCACCCTGGTGGTCATGGCGAT | |
| | GGTGAAGAGGAAAAACTCT | |
| Plasmid | ATGGCGACAAGACTGCTGTGCTGGGCGGCGCTGTGCCTGCTGGGAGCGGAACTGACTGAAG | 324 |
| BPAT15 | CGGGGGTCGCGCAGAGCCCTCGATACAAAATCATTGAGAAGCGGCAGTCTGTGGCGTTCTG | |
| | GTGCAACCCAATCAGCGGACACGCGACCCTGTACTGGTATCAGCAGATCCTGGGCCAGGGC | |
| | CCTAAGCTGCTGATTCAGTTCCAGAACAATGGCGTGGTGGACGATAGCCAGCTGCCAAAAG | |
| | ATAGATTTTCCGCGGAGAGGCTGAAGGGCGTGGACTCTACACTGAAAATTCAGCCTGCGAA | |
| | GCTGGAGGATAGCGCGGTGTACCTGTGCGCGAGCTCCCTGGACCCAGGCGATACCGGAGAG | |
| | CTGTTCTTTGGAGAGGGCAGCCGGCTGACAGTGCTGGAGGACCTGAGGAACGTGACCCCAC | |
| | CTAAAGTGAGCCTGTTCGAGCCATCCAAGGCGGAGATCGCGAATAAGCAGAAAGCGACCCT | |
| | GGTGTGCCTGGCGAGGGGCTTCTTTCCCGATCACGTGGAGCTGTCCTGGTGGGTGAACGGC | |
| | AAAGAGGTGCACTCTGGCGTGTGCACAGACCCTCAGGCGTACAAGGAGAGCAATTACTCCT | |
| | ATTGTCTGTCTAGCAGACTGAGGGTGAGCGCGACCTTTTGGCACAACCCCCGGAATCACTT | |
| | CCGCTGCCAGGTGCAGTTTCACGGCCTGTCCGAGGAGGATAAATGGCCTGAGGGCTCTCCA | |
| | AAGCCCGTGACACAGAATATCAGCGCGGAGGCGTGGGGAAGAGCGGACTGTGGCATTACAA | |
| | GCGCGTCCTATCAGCAGGGCGTGCTGTCCGCGACCATCCTGTACGAGATTCTGCTGGGCAA | |
| | GGCGACACTGTATGCGGTGCTGGTGTCCACCCTGGTGGTCATGGCGATGGTGAAGAGGAAA | |
| | AACTCTCGGGCGAAACGCTCTGGAAGCGGAGCGACCAATTTCAGCCTGCTGAAGCAGGCGG | |
| | GCGATGTGGAGGAGAACCCTGGCCCAATGGAGTCCTTTCTGGGCGGCGTGCTGCTGATCCT | |
| | GTGGCTGCAGGTGGACTGGGTGAAATCCCAGAAGATCGAGCAGAACTCTGAGGCGCTGAAT | |
| | ATTCAGGAGGGCAAGACCGCGACACTGACCTGCAACTACACAAATTATTCCCCAGCGTACC | |
| | TGCAGTGGTATAGGCAGGACCCAGGCAGGGGACCCGTGTTTCTGCTGCTGATTCGGGAGAA | |
| | TGAGAAGGAGAAAAGAAAGGAGAGGCTGAAAGTGACCTTCGATACCACACTGAAGCAGTCT | |
| | CTGTTTCACATCACAGCGTCTCAGCCAGCGGACAGCGCGACCTACCTGTGCGCGCTGGACA | |
| | TCTACCCTCACGATATGAGATTCGGCGCGGGCACAAGGCTGACCGTGAAACCAAACATCCA | |
| | GAATCCCGAGCCTGCGGTGTACCAGCTGAAGGACCCCCGCTCTCAGGATAGCACACTGTGC | |
| | CTGTTCACCGACTTTGATAGCCAGATCAACGTGCCTAAAACAATGGAGTCCGGCACCTTCA | |
| | TCACCGACAAGTGCGTGCTGGATATGAAAGCGATGGACTCCAAGTCTAACGGCGCGATCGC | |
| | GTGGTCCAATCAGACATCTTTCACCTGCCAGGATATCTTCAAGGAGACAAACGCGACCTAT | |
| | CCTTCCTCTGACGTGCCATGTGATGCGACACTGACCGAGAAGAGCTTCGAGACAGACATGA | |
| | ACCTGAATTTTCAGAATCTGCTGGTCATCGTGCTGAGAATCCTGCTGCTGAAGGTGGCGGG | |
| | CTTTAATCTGCTGATGACACTGCGGCTGTGGAGTTCCCGGGCGAAACGCTCTGGAAGCGGA | |
| | GAGGGCAGAGGAAGTCTTCTAACATGCGGTGACGTGGAGGAGAATCCCGGCCCTATGGATT | |
| | GGACCTGGATTCTGTTTCTGGTGGCCGCTGCCACAAGAGTGCACAGCAACTGGGTGAATGT | |
| | GATCAGCGACCTGAAGAAGATCGAGGATCTGATCCAGAGCATGCACATTGATGCCACCCTG | |
| | TACACAGAATCTGATGTGCACCCTAGCTGTAAAGTGACCGCCATGAAGTGTTTTCTGCTGG | |
| | AGCTGCAGGTGATTTCTCTGGAAAGCGGAGATGCCTCTATCCACGACACAGTGGAGAATCT | |
| | GATCATCCTGGCCAACAATAGCCTGAGCAGCAATGGCAATGTGACAGAGTCTGGCTGTAAG | |
| | GAGTGTGAGGAGCTGGAGGAGAAGAACATCAAGGAGTTTCTGCAGAGCTTTGTGCACATCG | |
| | TGCAGATGTTCATCAATACAAGCTCTGGCGGAGGATCTGGAGGAGGCGGATCTGGAGGAGG | |
| | AGGCAGTGGAGGCGGAGGATCTGGCGGAGGATCTCTGCAGATTACATGCCCTCCTCCAATG | |
| | TCTGTGGAGCACGCCGATATTTGGGTGAAGTCCTACAGCCTGTACAGCAGAGAGAGATACA | |
| | TCTGCAACAGCGGCTTTAAGAGAAAGGCCGGCACCTCTTCTCTGACAGAGTGCGTGCTGAA | |
| | TAAGGCCACAAATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTGACACCTCAGC | |
| | CTGAATCTCTGAGCCCTTCTGGAAAAGAACCTGCCGCCAGCTCTCCTAGCTCTAATAATAC | |
| | CGCCGCCACAACAGCCGCCATTGTGCCTGGATCTCAGCTGATGCCTAGCAAGTCTCCTAGC | |
| | ACAGGCACAACAGAGATCAGCAGCCACGAATCTTCTCACGGAACACCTTCTCAGACCACCG | |
| | CCAAGAATTGGGAGCTGACAGCCTCTGCCTCTCACCAGCCTCCAGGAGTGTATCCTCAGGG | |
| | CCACTCTGATACAACAGTGGCCATCAGCACATCTACAGTGCTGCTGTGTGGACTGTCTGCC | |
| | GTGTCTCTGCTGGCCTGTTACCTGAAGTCTAGACAGACACCTCCTCTGGCCTCTGTGGAGA | |
| | TGGAGGCCATGGAAGCCCTGCCTGTGACATGGGGAACAAGCAGCAGAGATGAAGACCTGGA | |
| | GAATTGTTCTCACCACCTG | |
| Plasmid | ATGGCGACAAGACTGCTGTGCTGGGCGGCGCTGTGCCTGCTGGGAGCGGAACTGACTGAAG | 325 |
| BTAP15 | CGGGGGTCGCGCAGAGCCCTCGATACAAAATCATTGAGAAGCGGCAGTCTGTGGCGTTCTG | |
| | GTGCAACCCAATCAGCGGACACGCGACCCTGTACTGGTATCAGCAGATCCTGGGCCAGGGC | |
| | CCTAAGCTGCTGATTCAGTTCCAGAACAATGGCGTGGTGGACGATAGCCAGCTGCCAAAAG | |
| | ATAGATTTTCCGCGGAGAGGCTGAAGGGCGTGGACTCTACACTGAAAATTCAGCCTGCGAA | |
| | GCTGGAGGATAGCGCGGTGTACCTGTGCGCGAGCTCCCTGGACCCAGGCGATACCGGAGAG | |
| | CTGTTCTTTGGAGAGGGCAGCCGGCTGACAGTGCTGGAGGACCTGAGGAACGTGACCCCAC | |
| | CTAAAGTGAGCCTGTTCGAGCCATCCAAGGCGGAGATCGCGAATAAGCAGAAAGCGACCCT | |
| | GGTGTGCCTGGCGAGGGGCTTCTTTCCCGATCACGTGGAGCTGTCCTGGTGGGTGAACGGC | |
| | AAAGAGGTGCACTCTGGCGTGTGCACAGACCCTCAGGCGTACAAGGAGAGCAATTACTCCT | |
| | ATTGTCTGTCTAGCAGACTGAGGGTGAGCGCGACCTTTTGGCACAACCCCCGGAATCACTT | |
| | CCGCTGCCAGGTGCAGTTTCACGGCCTGTCCGAGGAGGATAAATGGCCTGAGGGCTCTCCA | |
| | AAGCCCGTGACACAGAATATCAGCGCGGAGGCGTGGGGAAGAGCGGACTGTGGCATTACAA | |
| | GCGCGTCCTATCAGCAGGGCGTGCTGTCCGCGACCATCCTGTACGAGATTCTGCTGGGCAA | |
| | GGCGACACTGTATGCGGTGCTGGTGTCCACCCTGGTGGTCATGGCGATGGTGAAGAGGAAA | |
| | AACTCTCGGGCGAAACGCTCTGGAAGCGGAGAGGGCAGAGGAAGTCTTCTAACATGCGGTG | |
| | ACGTGGAGGAGAATCCCGGCCCTATGGAGTCCTTTCTGGGCGGCGTGCTGCTGATCCTGTG | |
| | GCTGCAGGTGGACTGGGTGAAATCCCAGAAGATCGAGCAGAACTCTGAGGCGCTGAATATT | |
| | CAGGAGGGCAAGACCGCGACACTGACCTGCAACTACACAAATTATTCCCCAGCGTACCTGC | |
| | AGTGGTATAGGCAGGACCCAGGCAGGGGACCCGTGTTTCTGCTGCTGATTCGGGAGAATGA | |
| | GAAGGAGAAAAGAAAGGAGAGGCTGAAAGTGACCTTCGATACCACACTGAAGCAGTCTCTG | |
| | TTTCACATCACAGCGTCTCAGCCAGCGGACAGCGCGACCTACCTGTGCGCGCTGGACATCT | |
| | ACCCTCACGATATGAGATTCGGCGCGGGCACAAGGCTGACCGTGAAACCAAACATCCAGAA | |
| | TCCCGAGCCTGCGGTGTACCAGCTGAAGGACCCCCGCTCTCAGGATAGCACACTGTGCCTG | |
| | TTCACCGACTTTGATAGCCAGATCAACGTGCCTAAAACAATGGAGTCCGGCACCTTCATCA | |
| | CCGACAAGTGCGTGCTGGATATGAAAGCGATGGACTCCAAGTCTAACGGCGCGATCGCGTG | |
| | GTCCAATCAGACATCTTTCACCTGCCAGGATATCTTCAAGGAGACAAACGCGACCTATCCT | |
| | TCCTCTGACGTGCCATGTGATGCGACACTGACCGAGAAGAGCTTCGAGACAGACATGAACC | |
| | TGAATTTTCAGAATCTGCTGGTCATCGTGCTGAGAATCCTGCTGCTGAAGGTGGCGGGCTT | |
| | TAATCTGCTGATGACACTGCGGCTGTGGAGTTCCCGGGCGAAACGCTCTGGAAGCGGAGCG | |
| | ACCAATTTCAGCCTGCTGAAGCAGGCGGGCGATGTGGAGGAGAACCCTGGCCCAATGGATT | |
| | GGACCTGGATTCTGTTTCTGGTGGCCGCTGCCACAAGAGTGCACAGCAACTGGGTGAATGT | |
| | GATCAGCGACCTGAAGAAGATCGAGGATCTGATCCAGAGCATGCACATTGATGCCACCCTG | |
| | TACACAGAATCTGATGTGCACCCTAGCTGTAAAGTGACCGCCATGAAGTGTTTTCTGCTGG | |
| | AGCTGCAGGTGATTTCTCTGGAAAGCGGAGATGCCTCTATCCACGACACAGTGGAGAATCT | |
| | GATCATCCTGGCCAACAATAGCCTGAGCAGCAATGGCAATGTGACAGAGTCTGGCTGTAAG | |
| | GAGTGTGAGGAGCTGGAGGAGAAGAACATCAAGGAGTTTCTGCAGAGCTTTGTGCACATCG | |
| | TGCAGATGTTCATCAATACAAGCTCTGGCGGAGGATCTGGAGGAGGCGGATCTGGAGGAGG | |
| | AGGCAGTGGAGGCGGAGGATCTGGCGGAGGATCTCTGCAGATTACATGCCCTCCTCCAATG | |
| | TCTGTGGAGCACGCCGATATTTGGGTGAAGTCCTACAGCCTGTACAGCAGAGAGAGATACA | |
| | TCTGCAACAGCGGCTTTAAGAGAAAGGCCGGCACCTCTTCTCTGACAGAGTGCGTGCTGAA | |
| | TAAGGCCACAAATGTGGCCCACTGGACAACACCTAGCCTGAAGTGCATTAGAGATCCTGCC | |
| | CTGGTCCACCAGAGGCCTGCCCCTCCATCTACAGTGACAACAGCCGGAGTGACACCTCAGC | |
| | CTGAATCTCTGAGCCCTTCTGGAAAAGAACCTGCCGCCAGCTCTCCTAGCTCTAATAATAC | |
| | CGCCGCCACAACAGCCGCCATTGTGCCTGGATCTCAGCTGATGCCTAGCAAGTCTCCTAGC | |
| | ACAGGCACAACAGAGATCAGCAGCCACGAATCTTCTCACGGAACACCTTCTCAGACCACCG | |
| | CCAAGAATTGGGAGCTGACAGCCTCTGCCTCTCACCAGCCTCCAGGAGTGTATCCTCAGGG | |
| | CCACTCTGATACAACAGTGGCCATCAGCACATCTACAGTGCTGCTGTGTGGACTGTCTGCC | |
| | GTGTCTCTGCTGGCCTGTTACCTGAAGTCTAGACAGACACCTCCTCTGGCCTCTGTGGAGA | |
| | TGGAGGCCATGGAAGCCCTGCCTGTGACATGGGGAACAAGCAGCAGAGATGAAGACCTGGA | |
| | GAATTGTTCTCACCACCTG | |
| Plasmid | ATGGCGACAAGACTGCTGTGCTGGGCGGCGCTGTGCCTGCTGGGAGCGGAACTGACTGAAG | 326 |
| BP15TA | CGGGGGTCGCGCAGAGCCCTCGATACAAAATCATTGAGAAGCGGCAGTCTGTGGCGTTCTG | |
| | GTGCAACCCAATCAGCGGACACGCGACCCTGTACTGGTATCAGCAGATCCTGGGCCAGGGC | |
| | CCTAAGCTGCTGATTCAGTTCCAGAACAATGGCGTGGTGGACGATAGCCAGCTGCCAAAAG | |
| | ATAGATTTTCCGCGGAGAGGCTGAAGGGCGTGGACTCTACACTGAAAATTCAGCCTGCGAA | |
| | GCTGGAGGATAGCGCGGTGTACCTGTGCGCGAGCTCCCTGGACCCAGGCGATACCGGAGAG | |
| | CTGTTCTTTGGAGAGGGCAGCCGGCTGACAGTGCTGGAGGACCTGAGGAACGTGACCCCAC | |
| | CTAAAGTGAGCCTGTTCGAGCCATCCAAGGCGGAGATCGCGAATAAGCAGAAAGCGACCCT | |
| | GGTGTGCCTGGCGAGGGGCTTCTTTCCCGATCACGTGGAGCTGTCCTGGTGGGTGAACGGC | |
| | AAAGAGGTGCACTCTGGCGTGTGCACAGACCCTCAGGCGTACAAGGAGAGCAATTACTCCT | |
| | ATTGTCTGTCTAGCAGACTGAGGGTGAGCGCGACCTTTTGGCACAACCCCCGGAATCACTT | |
| | CCGCTGCCAGGTGCAGTTTCACGGCCTGTCCGAGGAGGATAAATGGCCTGAGGGCTCTCCA | |
| | AAGCCCGTGACACAGAATATCAGCGCGGAGGCGTGGGGAAGAGCGGACTGTGGCATTACAA | |
| | GCGCGTCCTATCAGCAGGGCGTGCTGTCCGCGACCATCCTGTACGAGATTCTGCTGGGCAA | |
| | GGCGACACTGTATGCGGTGCTGGTGTCCACCCTGGTGGTCATGGCGATGGTGAAGAGGAAA | |
| | AACTCTCGGGCGAAACGCTCTGGAAGCGGAGCGACCAATTTCAGCCTGCTGAAGCAGGCGG | |
| | GCGATGTGGAGGAGAACCCTGGCCCAATGGATTGGACCTGGATTCTGTTTCTGGTGGCCGC | |
| | TGCCACAAGAGTGCACAGCAACTGGGTGAATGTGATCAGCGACCTGAAGAAGATCGAGGAT | |
| | CTGATCCAGAGCATGCACATTGATGCCACCCTGTACACAGAATCTGATGTGCACCCTAGCT | |
| | GTAAAGTGACCGCCATGAAGTGTTTTCTGCTGGAGCTGCAGGTGATTTCTCTGGAAAGCGG | |
| | AGATGCCTCTATCCACGACACAGTGGAGAATCTGATCATCCTGGCCAACAATAGCCTGAGC | |
| | AGCAATGGCAATGTGACAGAGTCTGGCTGTAAGGAGTGTGAGGAGCTGGAGGAGAAGAACA | |
| | TCAAGGAGTTTCTGCAGAGCTTTGTGCACATCGTGCAGATGTTCATCAATACAAGCTCTGG | |
| | CGGAGGATCTGGAGGAGGCGGATCTGGAGGAGGAGGCAGTGGAGGCGGAGGATCTGGCGGA | |
| | GGATCTCTGCAGATTACATGCCCTCCTCCAATGTCTGTGGAGCACGCCGATATTTGGGTGA | |
| | AGTCCTACAGCCTGTACAGCAGAGAGAGATACATCTGCAACAGCGGCTTTAAGAGAAAGGC | |
| | CGGCACCTCTTCTCTGACAGAGTGCGTGCTGAATAAGGCCACAAATGTGGCCCACTGGACA | |
| | ACACCTAGCCTGAAGTGCATTAGAGATCCTGCCCTGGTCCACCAGAGGCCTGCCCCTCCAT | |
| | CTACAGTGACAACAGCCGGAGTGACACCTCAGCCTGAATCTCTGAGCCCTTCTGGAAAAGA | |
| | ACCTGCCGCCAGCTCTCCTAGCTCTAATAATACCGCCGCCACAACAGCCGCCATTGTGCCT | |
| | GGATCTCAGCTGATGCCTAGCAAGTCTCCTAGCACAGGCACAACAGAGATCAGCAGCCACG | |
| | AATCTTCTCACGGAACACCTTCTCAGACCACCGCCAAGAATTGGGAGCTGACAGCCTCTGC | |
| | CTCTCACCAGCCTCCAGGAGTGTATCCTCAGGGCCACTCTGATACAACAGTGGCCATCAGC | |
| | ACATCTACAGTGCTGCTGTGTGGACTGTCTGCCGTGTCTCTGCTGGCCTGTTACCTGAAGT | |
| | CTAGACAGACACCTCCTCTGGCCTCTGTGGAGATGGAGGCCATGGAAGCCCTGCCTGTGAC | |
| | ATGGGGAACAAGCAGCAGAGATGAAGACCTGGAGAATTGTTCTCACCACCTGCGGGCGAAA | |
| | CGCTCTGGAAGCGGAGAGGGCAGAGGAAGTCTTCTAACATGCGGTGACGTGGAGGAGAATC | |
| | CCGGCCCTATGGAGTCCTTTCTGGGCGGCGTGCTGCTGATCCTGTGGCTGCAGGTGGACTG | |
| | GGTGAAATCCCAGAAGATCGAGCAGAACTCTGAGGCGCTGAATATTCAGGAGGGCAAGACC | |
| | GCGACACTGACCTGCAACTACACAAATTATTCCCCAGCGTACCTGCAGTGGTATAGGCAGG | |
| | ACCCAGGCAGGGGACCCGTGTTTCTGCTGCTGATTCGGGAGAATGAGAAGGAGAAAAGAAA | |
| | GGAGAGGCTGAAAGTGACCTTCGATACCACACTGAAGCAGTCTCTGTTTCACATCACAGCG | |
| | TCTCAGCCAGCGGACAGCGCGACCTACCTGTGCGCGCTGGACATCTACCCTCACGATATGA | |
| | GATTCGGCGCGGGCACAAGGCTGACCGTGAAACCAAACATCCAGAATCCCGAGCCTGCGGT | |
| | GTACCAGCTGAAGGACCCCCGCTCTCAGGATAGCACACTGTGCCTGTTCACCGACTTTGAT | |
| | AGCCAGATCAACGTGCCTAAAACAATGGAGTCCGGCACCTTCATCACCGACAAGTGCGTGC | |
| | TGGATATGAAAGCGATGGACTCCAAGTCTAACGGCGCGATCGCGTGGTCCAATCAGACATC | |
| | TTTCACCTGCCAGGATATCTTCAAGGAGACAAACGCGACCTATCCTTCCTCTGACGTGCCA | |
| | TGTGATGCGACACTGACCGAGAAGAGCTTCGAGACAGACATGAACCTGAATTTTCAGAATC | |
| | TGCTGGTCATCGTGCTGAGAATCCTGCTGCTGAAGGTGGCGGGCTTTAATCTGCTGATGAC | |
| | ACTGCGGCTGTGGAGTTCC | |
| Plasmid | ATGGCGACAAGACTGCTGTGCTGGGCGGCGCTGTGCCTGCTGGGAGCGGAACTGACTGAAG | 327 |
| BT15PA | CGGGGGTCGCGCAGAGCCCTCGATACAAAATCATTGAGAAGCGGCAGTCTGTGGCGTTCTG | |
| | GTGCAACCCAATCAGCGGACACGCGACCCTGTACTGGTATCAGCAGATCCTGGGCCAGGGC | |
| | CCTAAGCTGCTGATTCAGTTCCAGAACAATGGCGTGGTGGACGATAGCCAGCTGCCAAAAG | |
| | ATAGATTTTCCGCGGAGAGGCTGAAGGGCGTGGACTCTACACTGAAAATTCAGCCTGCGAA | |
| | GCTGGAGGATAGCGCGGTGTACCTGTGCGCGAGCTCCCTGGACCCAGGCGATACCGGAGAG | |
| | CTGTTCTTTGGAGAGGGCAGCCGGCTGACAGTGCTGGAGGACCTGAGGAACGTGACCCCAC | |
| | CTAAAGTGAGCCTGTTCGAGCCATCCAAGGCGGAGATCGCGAATAAGCAGAAAGCGACCCT | |
| | GGTGTGCCTGGCGAGGGGCTTCTTTCCCGATCACGTGGAGCTGTCCTGGTGGGTGAACGGC | |
| | AAAGAGGTGCACTCTGGCGTGTGCACAGACCCTCAGGCGTACAAGGAGAGCAATTACTCCT | |
| | ATTGTCTGTCTAGCAGACTGAGGGTGAGCGCGACCTTTTGGCACAACCCCCGGAATCACTT | |
| | CCGCTGCCAGGTGCAGTTTCACGGCCTGTCCGAGGAGGATAAATGGCCTGAGGGCTCTCCA | |
| | AAGCCCGTGACACAGAATATCAGCGCGGAGGCGTGGGGAAGAGCGGACTGTGGCATTACAA | |
| | GCGCGTCCTATCAGCAGGGCGTGCTGTCCGCGACCATCCTGTACGAGATTCTGCTGGGCAA | |
| | GGCGACACTGTATGCGGTGCTGGTGTCCACCCTGGTGGTCATGGCGATGGTGAAGAGGAAA | |
| | AACTCTCGGGCGAAACGCTCTGGAAGCGGAGAGGGCAGAGGAAGTCTTCTAACATGCGGTG | |
| | ACGTGGAGGAGAATCCCGGCCCTATGGATTGGACCTGGATTCTGTTTCTGGTGGCCGCTGC | |
| | CACAAGAGTGCACAGCAACTGGGTGAATGTGATCAGCGACCTGAAGAAGATCGAGGATCTG | |
| | ATCCAGAGCATGCACATTGATGCCACCCTGTACACAGAATCTGATGTGCACCCTAGCTGTA | |
| | AAGTGACCGCCATGAAGTGTTTTCTGCTGGAGCTGCAGGTGATTTCTCTGGAAAGCGGAGA | |
| | TGCCTCTATCCACGACACAGTGGAGAATCTGATCATCCTGGCCAACAATAGCCTGAGCAGC | |
| | AATGGCAATGTGACAGAGTCTGGCTGTAAGGAGTGTGAGGAGCTGGAGGAGAAGAACATCA | |
| | AGGAGTTTCTGCAGAGCTTTGTGCACATCGTGCAGATGTTCATCAATACAAGCTCTGGCGG | |
| | AGGATCTGGAGGAGGCGGATCTGGAGGAGGAGGCAGTGGAGGCGGAGGATCTGGCGGAGGA | |
| | TCTCTGCAGATTACATGCCCTCCTCCAATGTCTGTGGAGCACGCCGATATTTGGGTGAAGT | |
| | CCTACAGCCTGTACAGCAGAGAGAGATACATCTGCAACAGCGGCTTTAAGAGAAAGGCCGG | |
| | CACCTCTTCTCTGACAGAGTGCGTGCTGAATAAGGCCACAAATGTGGCCCACTGGACAACA | |
| | CCTAGCCTGAAGTGCATTAGAGATCCTGCCCTGGTCCACCAGAGGCCTGCCCCTCCATCTA | |
| | CAGTGACAACAGCCGGAGTGACACCTCAGCCTGAATCTCTGAGCCCTTCTGGAAAAGAACC | |
| | TGCCGCCAGCTCTCCTAGCTCTAATAATACCGCCGCCACAACAGCCGCCATTGTGCCTGGA | |
| | TCTCAGCTGATGCCTAGCAAGTCTCCTAGCACAGGCACAACAGAGATCAGCAGCCACGAAT | |
| | CTTCTCACGGAACACCTTCTCAGACCACCGCCAAGAATTGGGAGCTGACAGCCTCTGCCTC | |
| | TCACCAGCCTCCAGGAGTGTATCCTCAGGGCCACTCTGATACAACAGTGGCCATCAGCACA | |
| | TCTACAGTGCTGCTGTGTGGACTGTCTGCCGTGTCTCTGCTGGCCTGTTACCTGAAGTCTA | |
| | GACAGACACCTCCTCTGGCCTCTGTGGAGATGGAGGCCATGGAAGCCCTGCCTGTGACATG | |
| | GGGAACAAGCAGCAGAGATGAAGACCTGGAGAATTGTTCTCACCACCTGCGGGCGAAACGC | |
| | TCTGGAAGCGGAGCGACCAATTTCAGCCTGCTGAAGCAGGCGGGCGATGTGGAGGAGAACC | |
| | CTGGCCCAATGGAGTCCTTTCTGGGCGGCGTGCTGCTGATCCTGTGGCTGCAGGTGGACTG | |
| | GGTGAAATCCCAGAAGATCGAGCAGAACTCTGAGGCGCTGAATATTCAGGAGGGCAAGACC | |
| | GCGACACTGACCTGCAACTACACAAATTATTCCCCAGCGTACCTGCAGTGGTATAGGCAGG | |
| | ACCCAGGCAGGGGACCCGTGTTTCTGCTGCTGATTCGGGAGAATGAGAAGGAGAAAAGAAA | |
| | GGAGAGGCTGAAAGTGACCTTCGATACCACACTGAAGCAGTCTCTGTTTCACATCACAGCG | |
| | TCTCAGCCAGCGGACAGCGCGACCTACCTGTGCGCGCTGGACATCTACCCTCACGATATGA | |
| | GATTCGGCGCGGGCACAAGGCTGACCGTGAAACCAAACATCCAGAATCCCGAGCCTGCGGT | |
| | GTACCAGCTGAAGGACCCCCGCTCTCAGGATAGCACACTGTGCCTGTTCACCGACTTTGAT | |
| | AGCCAGATCAACGTGCCTAAAACAATGGAGTCCGGCACCTTCATCACCGACAAGTGCGTGC | |
| | TGGATATGAAAGCGATGGACTCCAAGTCTAACGGCGCGATCGCGTGGTCCAATCAGACATC | |
| | TTTCACCTGCCAGGATATCTTCAAGGAGACAAACGCGACCTATCCTTCCTCTGACGTGCCA | |
| | TGTGATGCGACACTGACCGAGAAGAGCTTCGAGACAGACATGAACCTGAATTTTCAGAATC | |
| | TGCTGGTCATCGTGCTGAGAATCCTGCTGCTGAAGGTGGCGGGCTTTAATCTGCTGATGAC | |
| | ACTGCGGCTGTGGAGTTCC | |

The corresponding theoretical polypeptide translation product resulting from each ORF, not accounting for N-terminal signal sequence cleavage or ribosomal skipping at each P2A and T2A site, is shown in Table E3.

TABLE E3
Polypeptide sequences encoded by SB plasmid ORFs.

| | | SEQ |
|---|---|---|
| | | ID |
| Plasmid | Amino Acid Translation of ORF | NO: |
| Plasmid | MESFLGGVLLILWLQVDWVKSQKIEQNSEALNIQEGKTATLTCNYTNYSPAYLQWYRQDPG | 330 |
| APBT15 | RGPVFLLLIRENEKEKRKERLKVTFDTTLKQSLFHITASQPADSATYLCALDIYPHDMRFG | |
| | AGTRLTVKPNIQNPEPAVYQLKDPRSQDSTLCLFTDFDSQINVPKTMESGTFITDKCVLDM | |
| | KAMDSKSNGAIAWSNQTSFTCQDIFKETNATYPSSDVPCDATLTEKSFETDMNLNFQNLLV | |
| | IVLRILLLKVAGENLLMTLRLWSSRAKRSGSGATNFSLLKQAGDVEENPGPMATRLLCWAA | |
| | LCLLGAELTEAGVAQSPRYKIIEKRQSVAFWCNPISGHATLYWYQQILGQGPKLLIQFQNN | |
| | GVVDDSQLPKDRFSAERLKGVDSTLKIQPAKLEDSAVYLCASSLDPGDTGELFFGEGSRLT | |
| | VLEDLRNVTPPKVSLFEPSKAEIANKQKATLVCLARGFFPDHVELSWWVNGKEVHSGVCTD | |
| | PQAYKESNYSYCLSSRLRVSATFWHNPRNHFRCQVQFHGLSEEDKWPEGSPKPVTQNISAE | |
| | AWGRADCGITSASYQQGVLSATILYEILLGKATLYAVLVSTLVVMAMVKRKNSRAKRSGSG | |
| | EGRGSLLTCGDVEENPGPMDWTWILFLVAAATRVHSNWVNVISDLKKIEDLIQSMHIDATL | |
| | YTESDVHPSCKVTAMKCFLLELQVISLESGDASIHDTVENLIILANNSLSSNGNVTESGCK | |
| | ECEELEEKNIKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTPSLKCIRDPA | |
| | LVHQRPAPPSTVTTAGVTPQPESLSPSGKEPAASSPSSNNTAATTAAIVPGSQLMPSKSPS | |
| | TGTTEISSHESSHGTPSQTTAKNWELTASASHQPPGVYPQGHSDTTVAISTSTVLLCGLSA | |
| | VSLLACYLKSRQTPPLASVEMEAMEALPVTWGTSSRDEDLENCSHHL | |
| Plasmid | MESFLGGVLLILWLQVDWVKSQKIEQNSEALNIQEGKTATLTCNYTNYSPAYLQWYRQDPG | 331 |
| ATBP15 | RGPVFLLLIRENEKEKRKERLKVTFDTTLKQSLFHITASQPADSATYLCALDIYPHDMRFG | |
| | AGTRLTVKPNIQNPEPAVYQLKDPRSQDSTLCLFTDFDSQINVPKTMESGTFITDKCVLDM | |
| | KAMDSKSNGAIAWSNQTSFTCQDIFKETNATYPSSDVPCDATLTEKSFETDMNLNFQNLLV | |
| | IVLRILLLKVAGFNLLMTLRLWSSRAKRSGSGEGRGSLLTCGDVEENPGPMATRLLCWAAL | |
| | CLLGAELTEAGVAQSPRYKIIEKRQSVAFWCNPISGHATLYWYQQILGQGPKLLIQFQNNG | |
| | VVDDSQLPKDRFSAERLKGVDSTLKIQPAKLEDSAVYLCASSLDPGDTGELFFGEGSRLTV | |
| | LEDLRNVTPPKVSLFEPSKAEIANKQKATLVCLARGFFPDHVELSWWVNGKEVHSGVCTDP | |
| | QAYKESNYSYCLSSRLRVSATFWHNPRNHFRCQVQFHGLSEEDKWPEGSPKPVTQNISAEA | |
| | WGRADCGITSASYQQGVLSATILYEILLGKATLYAVLVSTLVVMAMVKRKNSRAKRSGSGA | |
| | TNFSLLKQAGDVEENPGPMDWTWILFLVAAATRVHSNWVNVISDLKKIEDLIQSMHIDATL | |
| | YTESDVHPSCKVTAMKCFLLELQVISLESGDASIHDTVENLIILANNSLSSNGNVTESGCK | |
| | ECEELEEKNIKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTPSLKCIRDPA | |
| | LVHQRPAPPSTVTTAGVTPQPESLSPSGKEPAASSPSSNNTAATTAAIVPGSQLMPSKSPS | |
| | TGTTEISSHESSHGTPSQTTAKNWELTASASHQPPGVYPQGHSDTTVAISTSTVLLCGLSA | |
| | VSLLACYLKSRQTPPLASVEMEAMEALPVTWGTSSRDEDLENCSHHL | |
| Plasmid | MESFLGGVLLILWLQVDWVKSQKIEQNSEALNIQEGKTATLTCNYTNYSPAYLQWYRQDPG | 332 |
| AP15TB | RGPVFLLLIRENEKEKRKERLKVTFDTTLKQSLFHITASQPADSATYLCALDIYPHDMRFG | |
| | AGTRLTVKPNIQNPEPAVYQLKDPRSQDSTLCLFTDFDSQINVPKTMESGTFITDKCVLDM | |
| | KAMDSKSNGAIAWSNQTSFTCQDIFKETNATYPSSDVPCDATLTEKSFETDMNLNFQNLLV | |
| | IVLRILLLKVAGFNLLMTLRLWSSRAKRSGSGATNFSLLKQAGDVEENPGPMDWTWILFLV | |
| | AAATRVHSNWVNVISDLKKIEDLIQSMHIDATLYTESDVHPSCKVTAMKCFLLELQVISLE | |
| | SGDASIHDTVENLIILANNSLSSNGNVTESGCKECEELEEKNIKEFLQSFVHIVQMFINTS | |
| | SGGGSGGGGSGGGGSGGGGSGGGSLQITCPPPMSVEHADIWVKSYSLYSRERYICNSGFKR | |
| | KAGTSSLTECVLNKATNVAHWTTPSLKCIRDPALVHQRPAPPSTVTTAGVTPQPESLSPSG | |
| | KEPAASSPSSNNTAATTAAIVPGSQLMPSKSPSTGTTEISSHESSHGTPSQTTAKNWELTA | |
| | SASHQPPGVYPQGHSDTTVAISTSTVLLCGLSAVSLLACYLKSRQTPPLASVEMEAMEALP | |
| | VTWGTSSRDEDLENCSHHLRAKRSGSGEGRGSLLTCGDVEENPGPMATRLLCWAALCLLGA | |
| | ELTEAGVAQSPRYKIIEKRQSVAFWCNPISGHATLYWYQQILGQGPKLLIQFQNNGVVDDS | |
| | QLPKDRFSAERLKGVDSTLKIQPAKLEDSAVYLCASSLDPGDTGELFFGEGSRLTVLEDLR | |
| | NVTPPKVSLFEPSKAEIANKQKATLVCLARGFFPDHVELSWWVNGKEVHSGVCTDPQAYKE | |
| | SNYSYCLSSRLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| Plasmid | MESFLGGVLLILWLQVDWVKSQKIEQNSEALNIQEGKTATLTCNYTNYSPAYLQWYRQDPG | 333 |
| AT15PB | RGPVFLLLIRENEKEKRKERLKVTFDTTLKQSLFHITASQPADSATYLCALDIYPHDMRFG | |
| | AGTRLTVKPNIQNPEPAVYQLKDPRSQDSTLCLFTDFDSQINVPKTMESGTFITDKCVLDM | |
| | KAMDSKSNGAIAWSNQTSFTCQDIFKETNATYPSSDVPCDATLTEKSFETDMNLNFQNLLV | |
| | IVLRILLLKVAGFNLLMTLRLWSSRAKRSGSGEGRGSLLTCGDVEENPGPMDWTWILFLVA | |
| | AATRVHSNWVNVISDLKKIEDLIQSMHIDATLYTESDVHPSCKVTAMKCFLLELQVISLES | |
| | GDASIHDTVENLIILANNSLSSNGNVTESGCKECEELEEKNIKEFLQSFVHIVQMFINTSS | |
| | GGGSGGGGSGGGGSGGGGSGGGSLQITCPPPMSVEHADIWVKSYSLYSRERYICNSGFKRK | |
| | AGTSSLTECVLNKATNVAHWTTPSLKCIRDPALVHQRPAPPSTVTTAGVTPQPESLSPSGK | |
| | EPAASSPSSNNTAATTAAIVPGSQLMPSKSPSTGTTEISSHESSHGTPSQTTAKNWELTAS | |
| | ASHQPPGVYPQGHSDTTVAISTSTVLLCGLSAVSLLACYLKSRQTPPLASVEMEAMEALPV | |
| | TWGTSSRDEDLENCSHHLRAKRSGSGATNFSLLKQAGDVEENPGPMATRLLCWAALCLLGA | |
| | ELTEAGVAQSPRYKIIEKRQSVAFWCNPISGHATLYWYQQILGQGPKLLIQFQNNGVVDDS | |
| | QLPKDRFSAERLKGVDSTLKIQPAKLEDSAVYLCASSLDPGDTGELFFGEGSRLTVLEDLR | |
| | NVTPPKVSLFEPSKAEIANKQKATLVCLARGFFPDHVELSWWVNGKEVHSGVCTDPQAYKE | |
| | SNYSYCLSSRLRVSATFWHNPRNHFRCQVQFHGLSEEDKWPEGSPKPVTQNISAEAWGRAD | |
| | CGITSASYQQGVLSATILYEILLGKATLYAVLVSTLVVMAMVKRKNS | |
| Plasmid | MATRLLCWAALCLLGAELTEAGVAQSPRYKIIEKRQSVAFWCNPISGHATLYWYQQILGQG | 334 |
| BPAT15 | PKLLIQFQNNGVVDDSQLPKDRFSAERLKGVDSTLKIQPAKLEDSAVYLCASSLDPGDTGE | |
| | LFFGEGSRLTVLEDLRNVTPPKVSLFEPSKAEIANKQKATLVCLARGFFPDHVELSWWVNG | |
| | KEVHSGVCTDPQAYKESNYSYCLSSRLRVSATFWHNPRNHFRCQVQFHGLSEEDKWPEGSP | |
| | KPVTQNISAEAWGRADCGITSASYQQGVLSATILYEILLGKATLYAVLVSTLVVMAMVKRK | |
| | NSRAKRSGSGATNFSLLKQAGDVEENPGPMESFLGGVLLILWLQVDWVKSQKIEQNSEALN | |
| | IQEGKTATLTCNYTNYSPAYLQWYRQDPGRGPVFLLLIRENEKEKRKERLKVTFDTTLKQS | |
| | LFHITASQPADSATYLCALDIYPHDMRFGAGTRLTVKPNIQNPEPAVYQLKDPRSQDSTLC | |
| | LFTDFDSQINVPKTMESGTFITDKCVLDMKAMDSKSNGAIAWSNQTSFTCQDIFKETNATY | |
| | PSSDVPCDATLTEKSFETDMNLNFQNLLVIVLRILLLKVAGENLLMTLRLWSSRAKRSGSG | |
| | EGRGSLLTCGDVEENPGPMDWTWILFLVAAATRVHSNWVNVISDLKKIEDLIQSMHIDATL | |
| | YTESDVHPSCKVTAMKCFLLELQVISLESGDASIHDTVENLIILANNSLSSNGNVTESGCK | |
| | ECEELEEKNIKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTPSLKCIRDPA | |
| | LVHQRPAPPSTVTTAGVTPQPESLSPSGKEPAASSPSSNNTAATTAAIVPGSQLMPSKSPS | |
| | TGTTEISSHESSHGTPSQTTAKNWELTASASHQPPGVYPQGHSDTTVAISTSTVLLCGLSA | |
| | VSLLACYLKSRQTPPLASVEMEAMEALPVTWGTSSRDEDLENCSHHL | |
| Plasmid | MATRLLCWAALCLLGAELTEAGVAQSPRYKIIEKRQSVAFWCNPISGHATLYWYQQILGQG | 335 |
| BTAP15 | PKLLIQFQNNGVVDDSQLPKDRFSAERLKGVDSTLKIQPAKLEDSAVYLCASSLDPGDTGE | |
| | LFFGEGSRLTVLEDLRNVTPPKVSLFEPSKAEIANKQKATLVCLARGFFPDHVELSWWVNG | |
| | KEVHSGVCTDPQAYKESNYSYCLSSRLRVSATFWHNPRNHFRCQVQFHGLSEEDKWPEGSP | |
| | KPVTQNISAEAWGRADCGITSASYQQGVLSATILYEILLGKATLYAVLVSTLVVMAMVKRK | |
| | NSRAKRSGSGEGRGSLLTCGDVEENPGPMESFLGGVLLILWLQVDWVKSQKIEQNSEALNI | |
| | QEGKTATLTCNYTNYSPAYLQWYRQDPGRGPVFLLLIRENEKEKRKERLKVTFDTTLKQSL | |
| | FHITASQPADSATYLCALDIYPHDMRFGAGTRLTVKPNIQNPEPAVYQLKDPRSQDSTLCL | |
| | FTDFDSQINVPKTMESGTFITDKCVLDMKAMDSKSNGAIAWSNQTSFTCQDIFKETNATYP | |
| | SSDVPCDATLTEKSFETDMNLNFQNLLVIVLRILLLKVAGENLLMTLRLWSSRAKRSGSGA | |
| | TNFSLLKQAGDVEENPGPMDWTWILFLVAAATRVHSNWVNVISDLKKIEDLIQSMHIDATL | |
| | YTESDVHPSCKVTAMKCFLLELQVISLESGDASIHDTVENLIILANNSLSSNGNVTESGCK | |
| | ECEELEEKNIKEFLQSFVHIVQMFINTSSGGGSGGGGSGGGGSGGGGSGGGSLQITCPPPM | |
| | SVEHADIWVKSYSLYSRERYICNSGFKRKAGTSSLTECVLNKATNVAHWTTPSLKCIRDPA | |
| | LVHQRPAPPSTVTTAGVTPQPESLSPSGKEPAASSPSSNNTAATTAAIVPGSQLMPSKSPS | |
| | TGTTEISSHESSHGTPSQTTAKNWELTASASHQPPGVYPQGHSDTTVAISTSTVLLCGLSA | |
| | VSLLACYLKSRQTPPLASVEMEAMEALPVTWGTSSRDEDLENCSHHL | |
| Plasmid | MATRLLCWAALCLLGAELTEAGVAQSPRYKIIEKRQSVAFWCNPISGHATLYWYQQILGQG | 336 |
| BP15TA | PKLLIQFQNNGVVDDSQLPKDRFSAERLKGVDSTLKIQPAKLEDSAVYLCASSLDPGDTGE | |
| | LFFGEGSRLTVLEDLRNVTPPKVSLFEPSKAEIANKQKATLVCLARGFFPDHVELSWWVNG | |
| | KEVHSGVCTDPQAYKESNYSYCLSSRLRVSATFWHNPRNHFRCQVQFHGLSEEDKWPEGSP | |
| | KPVTQNISAEAWGRADCGITSASYQQGVLSATILYEILLGKATLYAVLVSTLVVMAMVKRK | |
| | NSRAKRSGSGATNFSLLKQAGDVEENPGPMDWTWILFLVAAATRVHSNWVNVISDLKKIED | |
| | LIQSMHIDATLYTESDVHPSCKVTAMKCELLELQVISLESGDASIHDTVENLIILANNSLS | |
| | SNGNVTESGCKECEELEEKNIKEFLQSFVHIVQMFINTSSGGGSGGGGSGGGGSGGGGSGG | |
| | GSLQITCPPPMSVEHADIWVKSYSLYSRERYICNSGFKRKAGTSSLTECVLNKATNVAHWT | |
| | TPSLKCIRDPALVHQRPAPPSTVTTAGVTPQPESLSPSGKEPAASSPSSNNTAATTAAIVP | |
| | GSQLMPSKSPSTGTTEISSHESSHGTPSQTTAKNWELTASASHQPPGVYPQGHSDTTVAIS | |
| | TSTVLLCGLSAVSLLACYLKSRQTPPLASVEMEAMEALPVTWGTSSRDEDLENCSHHLRAK | |
| | RSGSGEGRGSLLTCGDVEENPGPMESFLGGVLLILWLQVDWVKSQKIEQNSEALNIQEGKT | |
| | ATLTCNYTNYSPAYLQWYRQDPGRGPVFLLLIRENEKEKRKERLKVTFDTTLKQSLFHITA | |
| | SQPADSATYLCALDIYPHDMRFGAGTRLTVKPNIQNPEPAVYQLKDPRSQDSTLCLFTDED | |
| | SQINVPKTMESGTFITDKCVLDMKAMDSKSNGAIAWSNQTSFTCQDIFKETNATYPSSDVP | |
| | CDATLTEKSFETDMNLNFQNLLVIVLRILLLKVAGENLLMTLRLWSS | |
| Plasmid | MATRLLCWAALCLLGAELTEAGVAQSPRYKIIEKRQSVAFWCNPISGHATLYWYQQILGQG | 337 |
| BT15PA | PKLLIQFQNNGVVDDSQLPKDRFSAERLKGVDSTLKIQPAKLEDSAVYLCASSLDPGDTGE | |
| | LFFGEGSRLTVLEDLRNVTPPKVSLFEPSKAEIANKQKATLVCLARGFFPDHVELSWWVNG | |
| | KEVHSGVCTDPQAYKESNYSYCLSSRLRVSATFWHNPRNHFRCQVQFHGLSEEDKWPEGSP | |
| | KPVTQNISAEAWGRADCGITSASYQQGVLSATILYEILLGKATLYAVLVSTLVVMAMVKRK | |
| | NSRAKRSGSGEGRGSLLTCGDVEENPGPMDWTWILFLVAAATRVHSNWVNVISDLKKIEDL | |
| | IQSMHIDATLYTESDVHPSCKVTAMKCFLLELQVISLESGDASIHDTVENLIILANNSLSS | |
| | NGNVTESGCKECEELEEKNIKEFLQSFVHIVQMFINTSSGGGSGGGGSGGGGSGGGGSGGG | |
| | SLQITCPPPMSVEHADIWVKSYSLYSRERYICNSGFKRKAGTSSLTECVLNKATNVAHWTT | |
| | PSLKCIRDPALVHQRPAPPSTVTTAGVTPQPESLSPSGKEPAASSPSSNNTAATTAAIVPG | |
| | SQLMPSKSPSTGTTEISSHESSHGTPSQTTAKNWELTASASHQPPGVYPQGHSDTTVAIST | |
| | STVLLCGLSAVSLLACYLKSRQTPPLASVEMEAMEALPVTWGTSSRDEDLENCSHHLRAKR | |
| | SGSGATNFSLLKQAGDVEENPGPMESFLGGVLLILWLQVDWVKSQKIEQNSEALNIQEGKT | |
| | ATLTCNYTNYSPAYLQWYRQDPGRGPVFLLLIRENEKEKRKERLKVTFDTTLKQSLFHITA | |
| | SQPADSATYLCALDIYPHDMRFGAGTRLTVKPNIQNPEPAVYQLKDPRSQDSTLCLFTDED | |
| | SQINVPKTMESGTFITDKCVLDMKAMDSKSNGAIAWSNQTSFTCQDIFKETNATYPSSDVP | |
| | CDATLTEKSFETDMNLNFQNLLVIVLRILLLKVAGENLLMTLRLWSS | |

For control purposes, three additional SB transposon plasmids were prepared. Plasmid 15 contains a monocistronic expression cassette, Cassette 15, encoding mbIL15. Plasmid APB contains a bicistronic expression cassette, Cassette APB, encoding TCRα (5′) and TCRβ (3′) with an intervening fP2A element. Plasmid BPA contains a bicistronic expression cassette, Cassette BPA, encoding TCRβ (5′) and TCRα (3′) with an intervening fP2A element. These expression cassettes, including suitable transcriptional regulatory elements, were inserted between the ITRs of SB transposon plasmids. The 5′ to 3′ order of elements in the ORF of each control expression cassette and SB Plasmid is shown in Table E4, and schematics of the ORFs of these three expression cassettes are shown in FIG. 2B.

TABLE E4
Control SB transposon plasmids.

| Plasmid Name | Cassette Name | Order of Elements (5′ to 3′) |
|---|---|---|
| Plasmid 15 | Cassette 15 | mbIL15 |
| Plasmid APB | Cassette APB | TCRα-fP2A-TCRβ |
| Plasmid BPA | Cassette BPA | TCRβ-fP2A-TCRα |

A plasmid encoding SB11 transposase, Plasmid TA, was also constructed.

### Example 2: Generation and Evaluation of T Cells

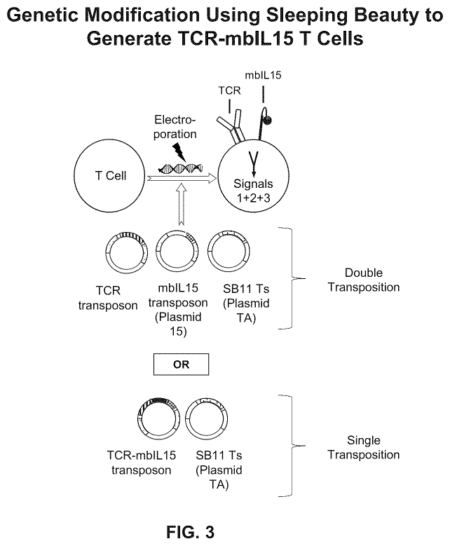

This Example describes the generation and evaluation of T cells co-expressing TCRα, TCRβ, and mbIL15 from the plasmids described in Example 1. A schematic of the gene transfer process for both double transposition (using separate plasmids encoding TCRα/TCRβ and mbIL15) and single transposition (using a tricistronic plasmid encoding TCRα/TCRβ and mbIL15 together) is shown in FIG. 3.

Briefly, peripheral blood mononuclear cells (PBMCs) were enriched from leukapheresis product obtained from a normal donor (Discovery Life Sciences, Austin, TX). The resulting PBMCs were collected, cryopreserved, and stored in the vapor phase of a liquid nitrogen tank.

To generate the TCR-T cells described in this Example 2, the plasmids described in Example 1 were electroporated into the enriched PBMCs. Briefly, cryopreserved PBMCs were thawed, resuspended in supplemented media, and incubated in a 37° C./5% CO₂ incubator for one hour. The PBMC test articles listed in Table E5 were then prepared.

TABLE E5
PBMC test articles.

| Group | Name | Description |
|---|---|---|
| 1 | NT cells | Non-transposed PBMCs |
| 2 | mbIL15 cells | PBMCs transposed with Plasmid 15 and Plasmid TA |
| 3 | APB cells | PBMCs transposed with Plasmid APB and Plasmid TA |
| 4 | BPA cells | PBMCs transposed with Plasmid BPA and Plasmid TA |
| 5 | APB+15 cells | PBMCs transposed with Plasmid APB, Plasmid 15, and Plasmid TA |
| 6 | BPA+15 cells | PBMCs transposed with Plasmid BPA, Plasmid 15, and Plasmid TA |
| 7 | APBT15 cells | PBMCs transposed with Plasmid APBT15 and Plasmid TA |
| 8 | ATBP15 cells | PBMCs transposed with Plasmid ATBP15 and Plasmid TA |
| 9 | AP15TB cells | PBMCs transposed with Plasmid AP15TB and Plasmid TA |
| 10 | AT15PB cells | PBMCs transposed with Plasmid AT15PB and Plasmid TA |
| 11 | BPAT15 cells | PBMCs transposed with Plasmid BPAT15 and Plasmid TA |
| 12 | BTAP15 cells | PBMCs transposed with Plasmid BTAP15 and Plasmid TA |
| 13 | BP15TA cells | PBMCs transposed with Plasmid BP15TA and Plasmid TA |
| 14 | BT15PA cells | PBMCs transposed with Plasmid BT15PA and Plasmid TA |

Test articles were prepared as follows:

Group 1: Rested cells were harvested, spun down, resuspended in supplemented media, and incubated in a 37° C./5% CO₂ incubator overnight.

Groups 2-14: Rested cells were harvested, spun down, resuspended in electroporation buffer together with the plasmids listed in Table E5, and electroporated. Following electroporation, cell suspensions were collected, transferred to supplemented media, and incubated in a 37° C./5% CO₂ incubator overnight.

Within 24 hours post-electroporation (Day 1), the cells were harvested from culture, counted, and sampled by flow cytometry to determine mbIL15 and TCR transgene expression. Briefly, up to 1×10⁶ cells of each test article were stained with human Fc Block (BD Biosciences 564220) first to reduce background staining for 10 minutes at room temperature. Cell suspensions were further stained with fluorochrome conjugated antibodies (listed in Table 1) diluted in Brilliant Stain Buffer (BD Biosciences 566349) for 30 minutes at 4° C. TCR expression was detected using Percp-Cy5.5 conjugated anti-mouse TCRβ antibody specific for the murine constant region of TCRβ. Other fluorescently conjugated antibodies used included: CD3 (Clone OKT-3), IL-15 (34559), CD8 (Clone RPA-T8), and Invitrogen violet live/Dead dye (Table E6).

TABLE E6
Fluorescently Conjugated Antibodies.

| Antibody Target | Clone | Fluorophore | Company & Cat # |
|---|---|---|---|
| Live/Dead | | Pacific Blue | Invitrogen L34955A |
| Human CD3 | OKT3 | BV510 | Biolegend 317332 |
| Human CD8 | RPA-T8 | PE-Cy7 | BD Biosciences 557746 |
| Human IL-15 | 34559 | PE | R&D Systems IC2471P |
| Mouse TCR | H57-597 | Percp-Cy5.5 | BD Biosciences 560657 |

Cells were washed with FACS buffer (PBS, 2% FBS, 0.1% sodium azide). Data were acquired using an NovoCyte Quanteon flow cytometer system (Agilent) and analyzed with FlowJo software (version 10.7.1; TreeStar, Ashland, OR) to determine the percentage of each transgenic subpopulation (mbIL15⁺mTCR⁺, mbIL15^{neg}mTCR⁺, mbIL15⁺mTCR^{neg}, mbIL15^{neg}mTCR^{neg}) present in each test article. Unless described otherwise, transgene expression was assessed on gated cell events, singlets, viable events, and CD3⁺ cells.

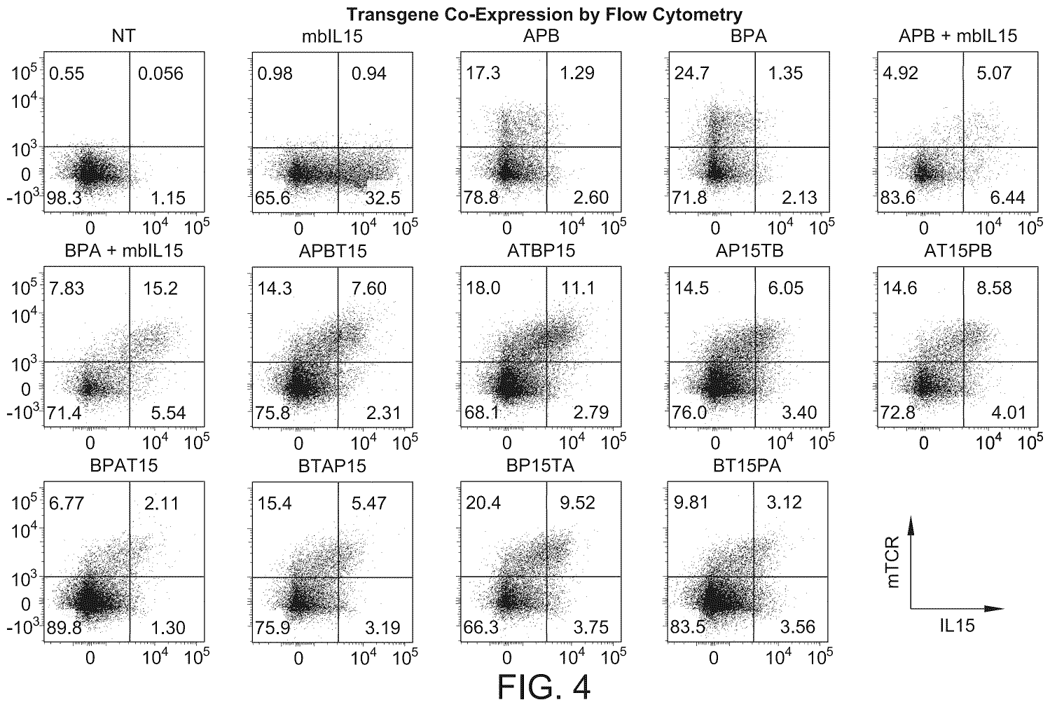

Results of flow cytometry are shown in FIG. 4 and Table E7.

TABLE E7
Day 1 post-electroporation specifications and transgene
expression of genetically modified T cells.

| | | Viability | mbIL15⁺ | mTCR⁺ | mbIL15⁺mTCR⁺ |
|---|---|---|---|---|---|
| Group | Name | (%) | (% CD3⁺) | (% CD3⁺) | (% CD3⁺) |
| 1 | NT cells | 97.5 | N/A | N/A | N/A |
| 2 | mbIL15 cells | 83.9 | 33.4 | N/A | N/A |
| 3 | APB cells | 68.3 | N/A | 18.6 | N/A |
| 4 | BPA cells | 75.4 | N/A | 26.1 | N/A |
| 5 | APB+15 cells | 65.7 | 11.5 | 9.99 | 5.07 |
| 6 | BPA+15 cells | 78.5 | 20.7 | 23.0 | 15.2 |
| 7 | APBT15 cells | 78.0 | 9.91 | 21.9 | 7.60 |
| 8 | ATBP15 cells | 82.7 | 13.9 | 29.1 | 11.1 |
| 9 | AP15TB cells | 84.1 | 9.45 | 20.6 | 6.05 |
| 10 | AT15PB cells | 81.6 | 12.6 | 23.2 | 8.58 |
| 11 | BPAT15 cells | 64.3 | 3.41 | 8.88 | 2.11 |
| 12 | BTAP15 cells | 73.8 | 8.66 | 20.9 | 5.47 |
| 13 | BP15TA cells | 83.7 | 13.3 | 29.9 | 9.52 |
| 14 | BT15PA cells | 74.5 | 6.68 | 12.9 | 3.12 |

### Example 3: Evaluation of Cell Culture Conditions for Recovery and Expansion of Transduced T Cells

This Example describes the evaluation of cell culture conditions for recovery and expansion of T cells electroporated to co-express TCRα, TCRβ, and mbIL15 from plasmids described in Example 1. TCR-T cells described in this Example 3 were generated similarly to those described in Example 2 except as indicated below.

Briefly, cryopreserved PBMCs were thawed, resuspended in 50:50 media and placed in a 37° C./5% CO₂ incubator before electroporation.

Test articles as listed below in Table E8 were then prepared as follows:

TABLE E8
PBMC test articles.

| Group | Name | Description |
|---|---|---|
| 1 | NT cells | Non-transduced PBMCs |
| 2 | BPA cells | PBMCs transduced with Plasmid |
| | | BPA and Plasmid TA |
| 3 | BPA+15 cells | PBMCs transduced with Plasmid |
| | | BPA, Plasmid 15, and Plasmid TA |

Group 1: Cells were harvested, spun down, resuspended in recovery media 1 (containing IL-2+IL-21, see below), and incubated in a 37° C./5% CO₂ incubator overnight.

Group 2: Cells were harvested, spun down, resuspended in electroporation buffer together with the plasmids listed in Table E8, and electroporated. Following electroporation, cell suspensions were collected, transferred to recovery media 1 (containing IL-2+IL-21, see below) and incubated in a 37° C./5% CO₂ incubator overnight.

Group 3: Cells were harvested, spun down, resuspended in electroporation buffer together with the plasmids listed in Table E8, and electroporated. Following electroporation, cell suspensions were collected, transferred to one of recovery media 1-9 (described below), and incubated in a 37° C./5% CO₂ incubator overnight.

The following recovery media were tested:
Recovery media 1: 50:50 media containing IL-2+IL-21
Recovery media 2: 50:50 media containing IL-2+IL-21+N-acetylcysteine (NAC)
Recovery media 3: 50:50 media containing NAC
Recovery media 4: 50:50 media containing IL-7
Recovery media 5: 50:50 media containing IL-7+NAC
Recovery media 6: 50:50 media containing IL-15
Recovery media 7: 50:50 media containing IL-15+NAC
Recovery media 8: 50:50 media containing IL-7+IL-15
Recovery media 9: 50:50 media containing IL-7+IL-15+NAC)

Within 24 hours post-electroporation (Day 1), mTCR positive (mTCR+) cells were isolated using mTCR antibody and MACS® Cell Separation system (Miltenyi Biotec). Live TCR+ cells that had been incubated in recovery media 9 were transferred to G-REX® culture plates (Wilson Wolf Manufacturing) and incubated with one of first expansion media 1-5 (described below) with irradiated feeder cells+OKT3 antibody. Live TCR+ cells from Group 2 were transferred to G-REX® culture plates and incubated with first expansion media 1 with irradiated feeder cells+OKT3 antibody. The same number of Group 1 cells were transferred to G-REX® culture plates (Wilson Wolf Manufacturing) and incubated with first expansion media 1 with irradiated feeder cells+OKT3 antibody.
First expansion media 1: 50:50 media containing IL-2+IL-21
First expansion media 2: 50:50 media containing IL-2
First expansion media 3: 50:50 media containing IL-21+IL-7*
First expansion media 4: 50:50 media containing IL-21+IL-7
First expansion media 5: 50:50 media containing IL-21+IL-7*+IL-12*

Where a “*” appears beside a cytokine, it indicates that the cytokine was added on day 1 only.

Cells were fed with 50:50 media containing the indicated cytokines regularly. After 13 days of first phase expansion, TCR+ cells were isolated with mTCR antibody. The isolated TCR+ T cells that had been incubated with first expansion media 5 were transferred to G-REX® culture plates (Wilson Wolf Manufacturing) and incubated with one of second expansion media 1-4 (described below) with irradiated feeder cells+OKT3 antibody.
Second expansion media 1: 50:50 media containing IL-2 (3000 U/ml)
Second expansion media 2: 50:50 media containing IL-2 (300 U/ml)
Second expansion media 3: 50:50 media containing IL-21+NAC+IL-12*
Second expansion media 4: 50:50 media containing IL-21+NAC+IL-2 (30 U/ml)+IL-12*

Where a “*” appears beside a cytokine, it indicates that the cytokine was added on day 1 of the second expansion phase only.

During this second expansion phase, cells were regularly fed with IL-2 (for the cells in second expansion media 1 and 2) or IL-21 (for the cells in second expansion media 3 and 4). After 15 days of second phase expansion, cells were harvested, and expression of mTCR and mbIL15 was detected on CD3+ gated population with mouse TCR beta antibody and IL-15 antibody as described in Example 2. Cell count and viability was accessed with a NC3000 cell counter. Unless described otherwise, transgene expression was assessed on gated cell events, singlets, viable events, and CD3⁺ cells.

Expression of mTCR and mbIL15 was assessed for each test article at three separate time points: 1) after electroporation (Day 1); 2) after first expansion phase (Day 13); and after second expansion phase (Day 28). Fold expansion was assessed for each test article after the second expansion phase (Day 28).

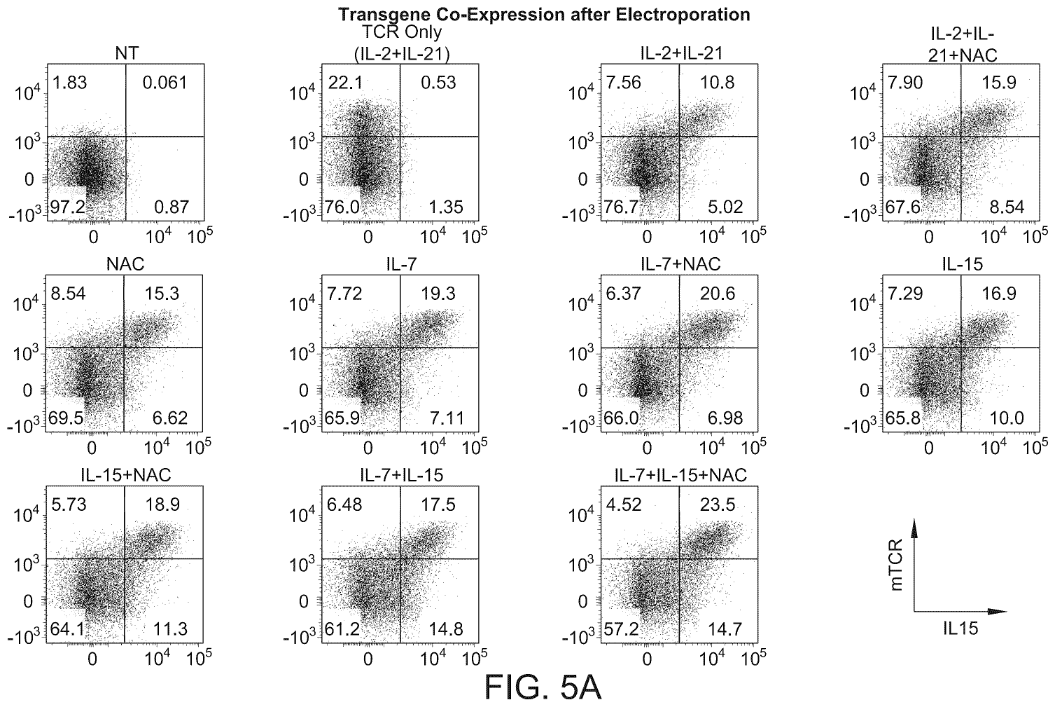

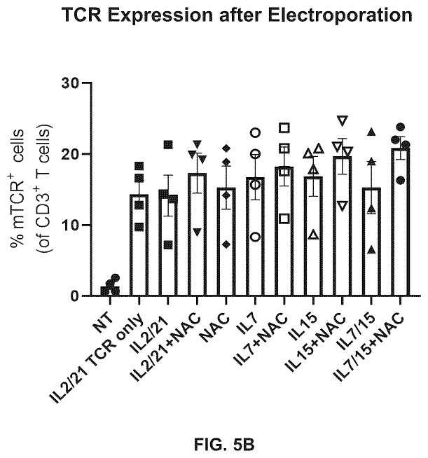

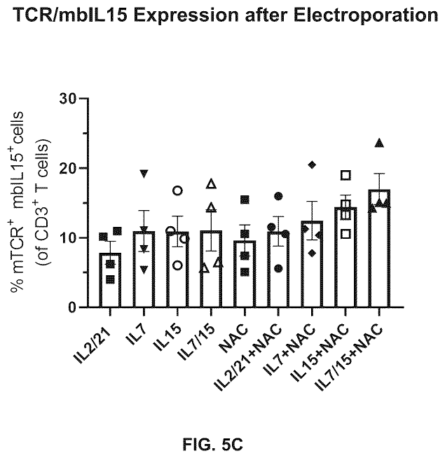

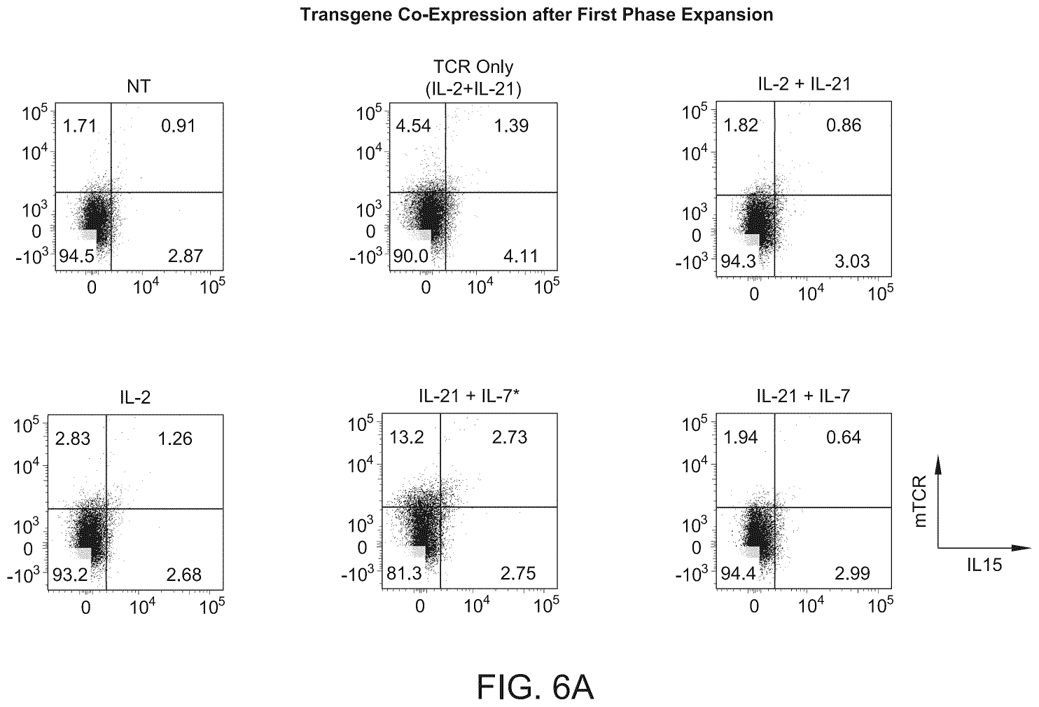

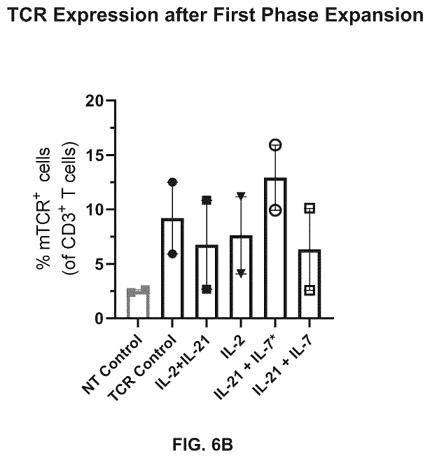

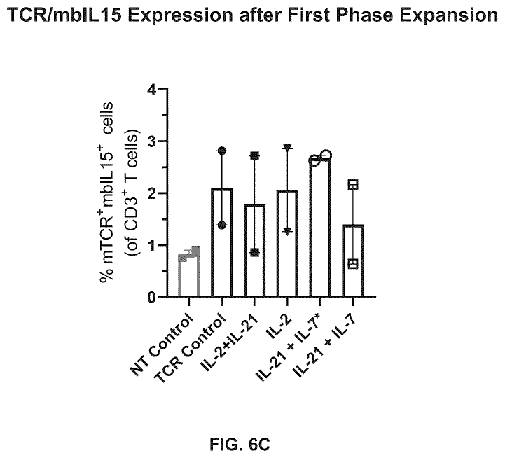

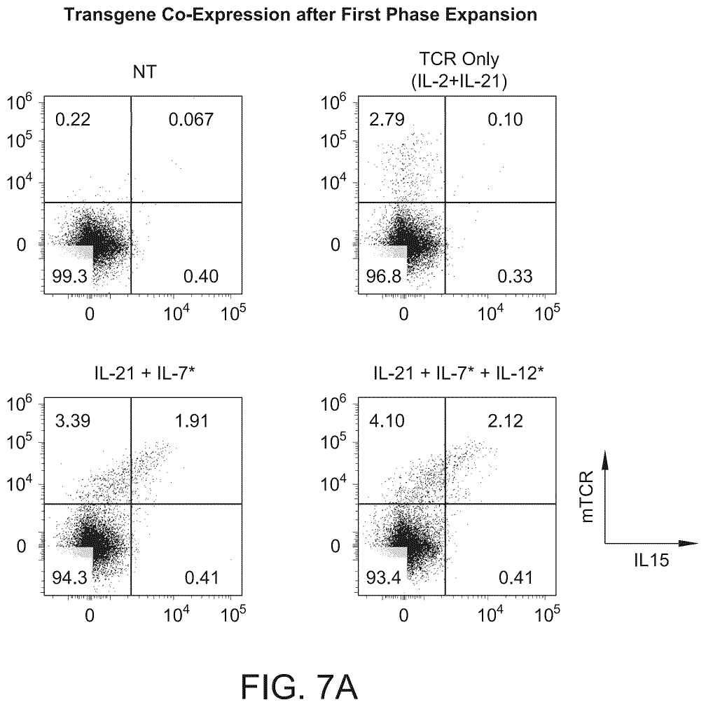

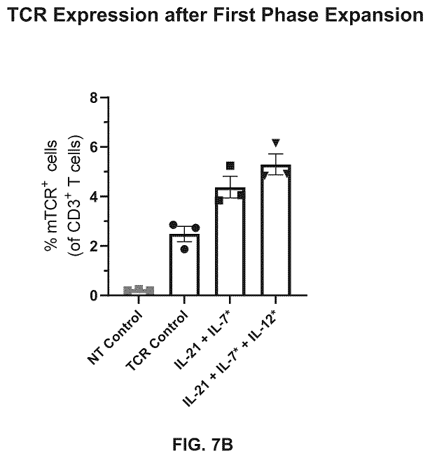

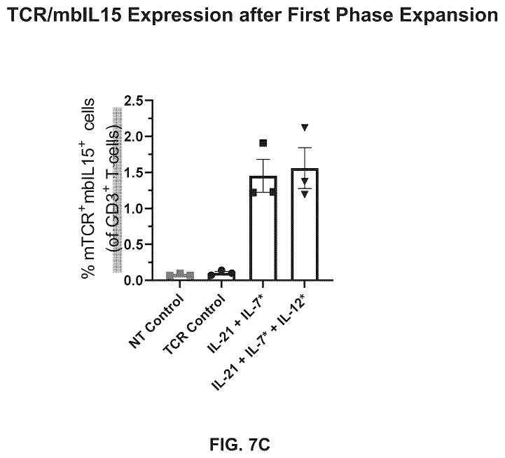

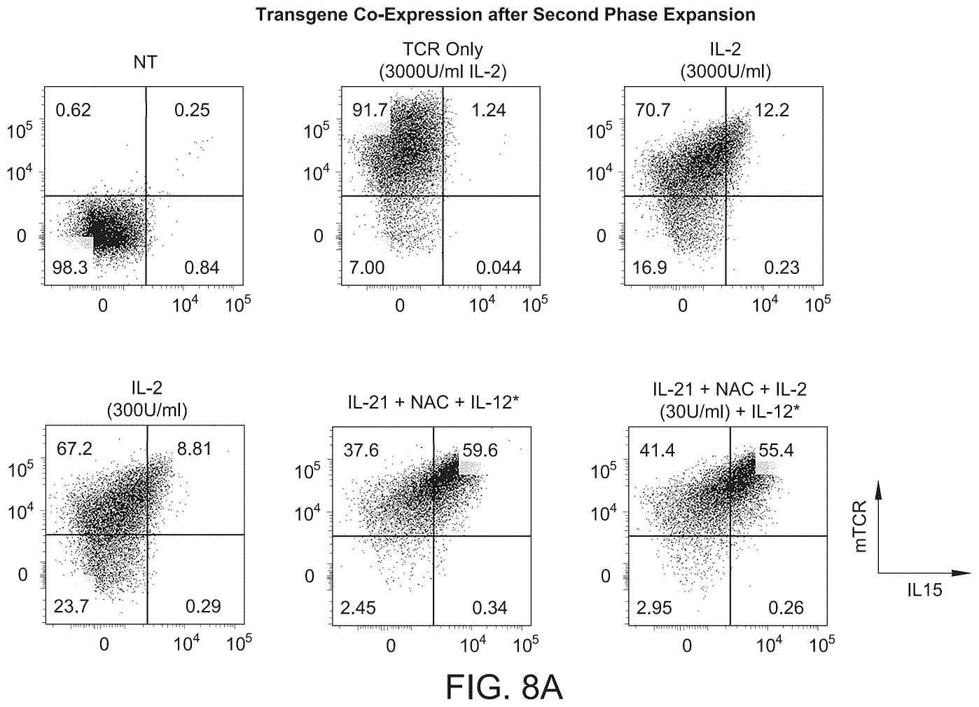

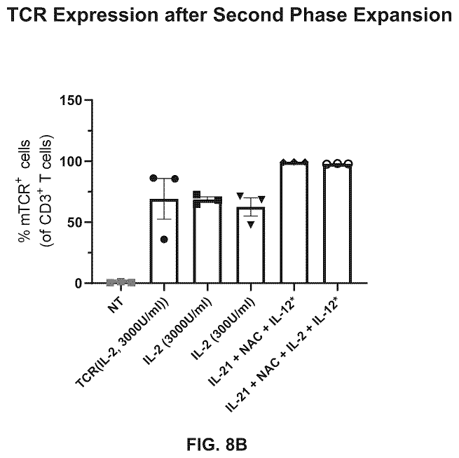

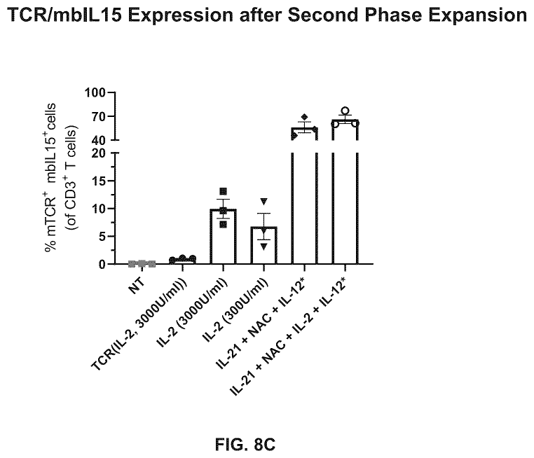

TCR and mbIL15 expression results detected by flow cytometry after electroporation are shown in FIG. 5A-5C. TCR and mbIL15 expression results detected by flow cytometry after the first expansion phase are shown in FIG. 6A-6C and FIG. 7A-7C. TCR and mbIL15 expression results detected by flow cytometry after the second expansion phase are shown in FIG. 8A-C.

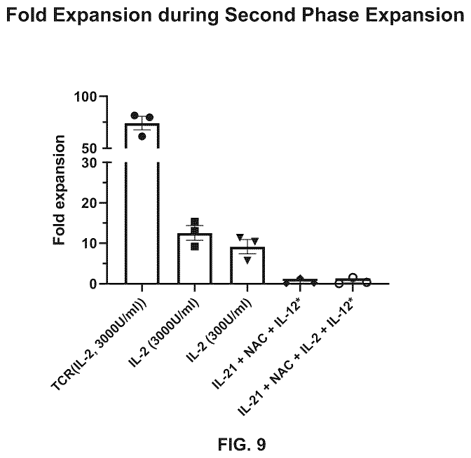

Fold expansion of cell number during the second expansion phase is shown in FIG. 9.

Conclusions: Addition of N-acetylcysteine (NAC) to overnight recovery culture media containing cytokines enhanced transgene expression after electroporation. Moreover, addition of IL-7 and IL-15 to recovery media enhanced the expression of transgene after electroporation. In case of IL-7, addition to first expansion media increased transgene expression after expansion phase, but only when added on the first day of the expansion phase. Compared to the IL-2 treatment, the incubation of cells with NAC and IL-21 significantly decreased fold expansion when added to cell culture media during second phase expansion. Similar to the addition of IL-7, when IL-12 was added to expansion media on the first day of the first expansion phase, increased transgene expression was seen.

### Example 4: Generation and Evaluation of Expanded T Cells

This Example describes the generation and evaluation of T cells co-expressing TCRα, TCRβ, and mbIL15 from the plasmids described in Example 1. TCR-T cells described in this Example 4 were generated similarly to those described in Example 2 except as indicated below.

Briefly, cryopreserved PBMCs were thawed, resuspended in supplemented media (IL-7+IL-15), and incubated in a 37° C./5% CO₂ incubator for one hour.

Test articles as listed above in Table E5 were then prepared as follows:

Group 1: Rested cells were harvested, spun down, resuspended in recovery media (50:50 media containing IL-7+IL-15+n-acetylcysteine (NAC)), and incubated in a 37° C./5% CO₂ incubator overnight.

Groups 2-14: Rested cells were harvested, spun down, resuspended in electroporation buffer together with the plasmids listed in Table E5, and electroporated. Following electroporation, cell suspensions were collected, transferred to recovery media (50:50 media containing IL-7+IL-15+NAC), and incubated in a 37° C./5% CO₂ incubator overnight.

Groups 3-14: Within 24 hours post-electroporation (Day 1), mTCR positive (mTCR+) cells were isolated using mTCR antibody and MACS® Cell Separation system (Miltenyi Biotec). Live cells from groups 1 & 2 and live TCR+ cells from groups 3-14 were transferred to G-REX® culture plates (Wilson Wolf Manufacturing) and incubated with a first expansion media (50:50 media containing IL-21+IL-7+OKT3+irradiated feeder cells).

Cells were fed with regularly with cytokines. After 13 days of first phase expansion, cells were harvested, and expression of mTCR and mbIL15 was detected on CD3+ gated population with mouse TCR beta antibody and IL-15 antibody as described in Example 2. Cell count and viability was accessed with a NC3000 cell counter. Unless described otherwise, transgene expression was assessed on gated cell events, singlets, viable events, and CD3⁺ cells.

Expression of mTCR and mbIL15 and cell viability was assessed for each test article at two separate time points: 1) after electroporation (Day 1), and 2) after first expansion phase (Day 13).

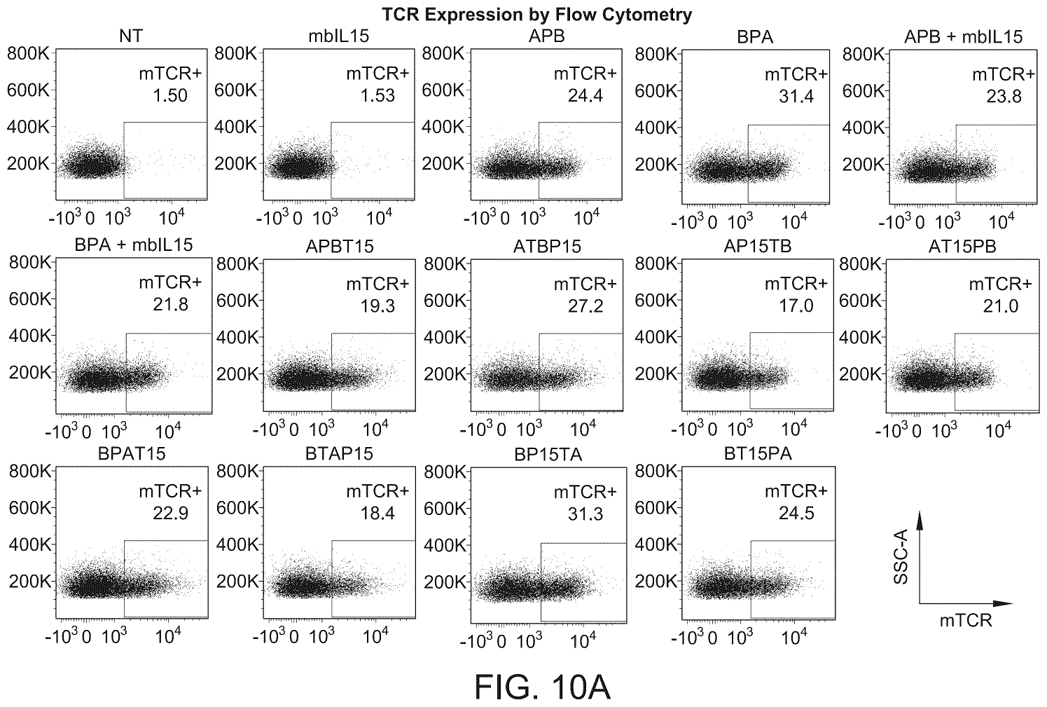

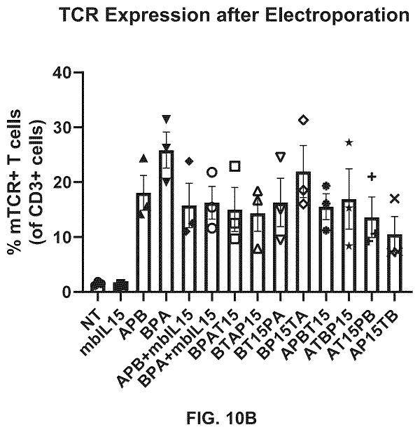

TCR expression after electroporation (Day 1) is shown in FIG. 10A-10B. FIG. 10A provides representative TCR expression data from each test article. FIG. 10B provides TCR expression data from three donors presented as % mTCR+ cells out of CD3+ cells.

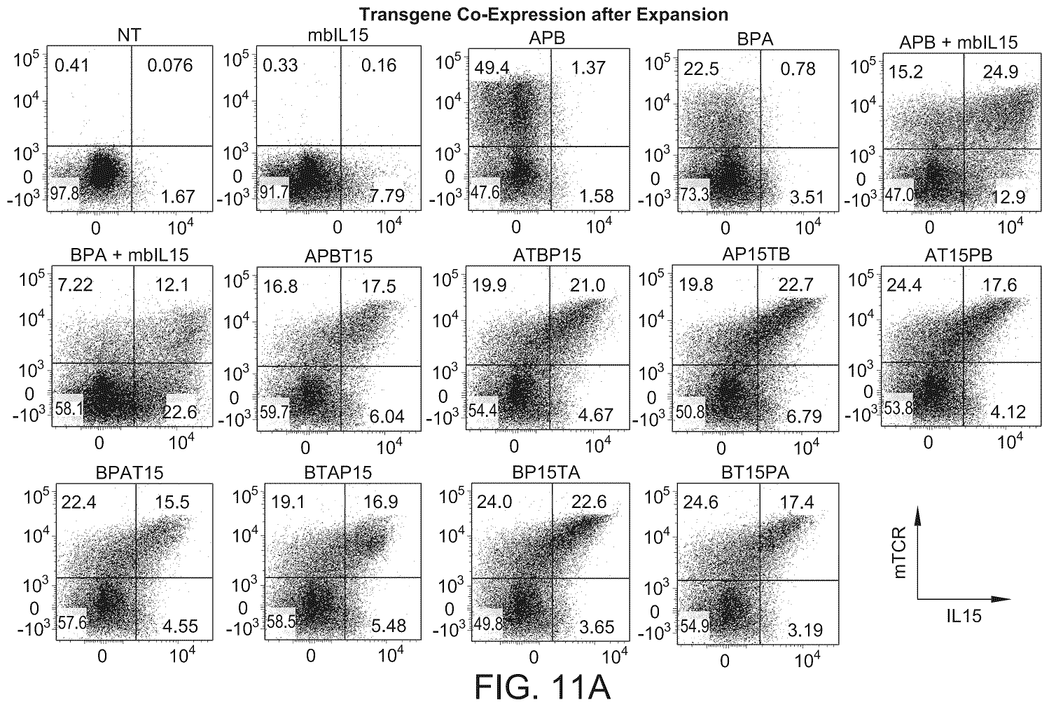

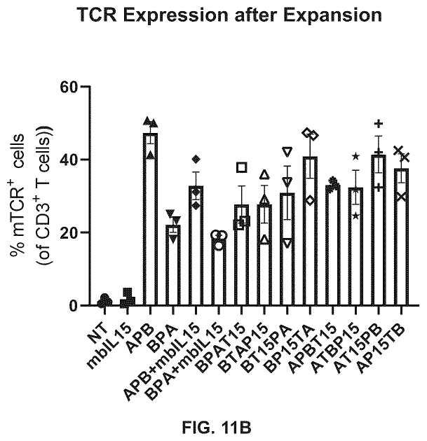

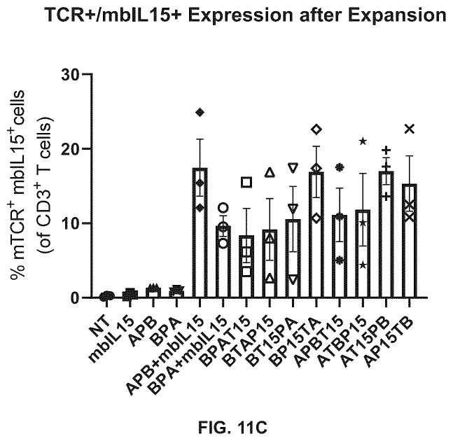

TCR and mbIL15 expression after first phase expansion (Day 13) is shown in FIG. 11A-11C. FIG. 11A provides representative TCR and mbIL15 expression data from each test article. FIG. 11B provides TCR expression data from three donors presented as % mTCR+ cells out of CD3+ cells and FIG. 11C provides % TCR+mbIL15+ cells out of CD3+ cells.

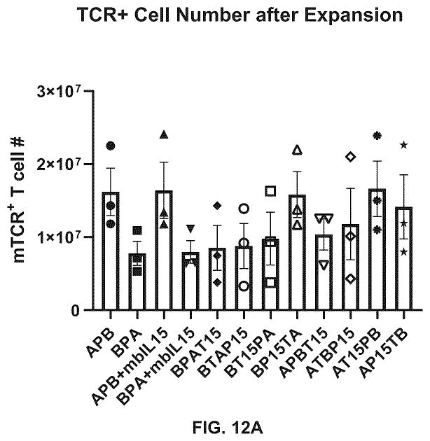

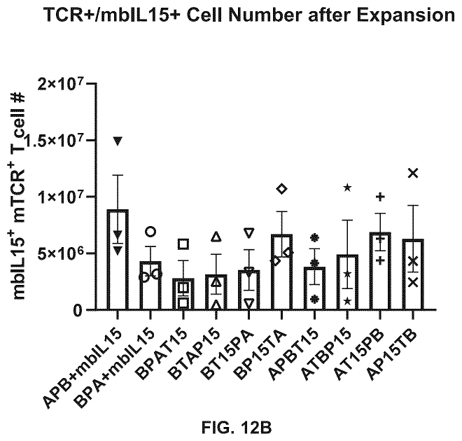

TCR+ and TCR+mbIL15+ cell number was also assessed after first phase expansion (Day 13) as shown in FIG. 12A-12B. FIG. 12A provides TCR expression data from three donors presented as total number of mTCR+ T cells and FIG. 12B provides total number of TCR+mbIL15+ T cells.

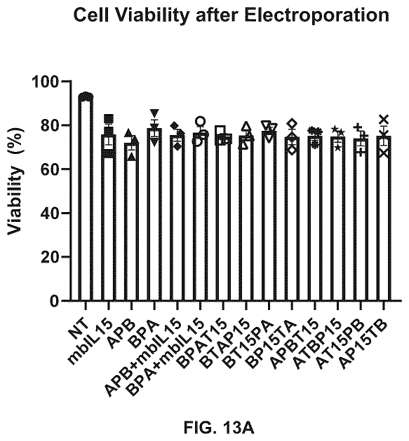

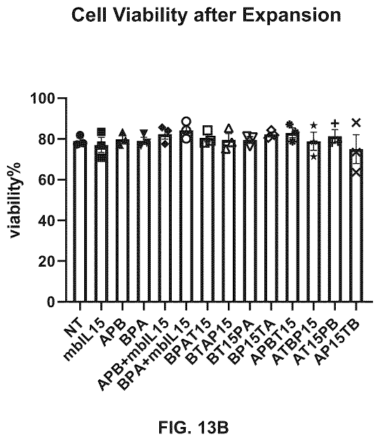

Cell viability after electroporation (Day 1) and after first phase expansion (Day 13) is shown in FIGS. 13A & 13B, respectively.

The transgene expression data and cell count data demonstrate that BP15TA and AP15 TB are the most potent candidates to have mbIL15+ TCR+ T cells with the highest level of TCR and mbIL15 expression. Viability data demonstrated that despite of the size of the tricistronic mbIL15+ TCR vectors (Groups 7-14), the viability is similar to the two-vector co-transfection system (Groups 5 & 6).

Functionality of the TCR-T cells was also measured following first phase expansion (Day 13) as described below.

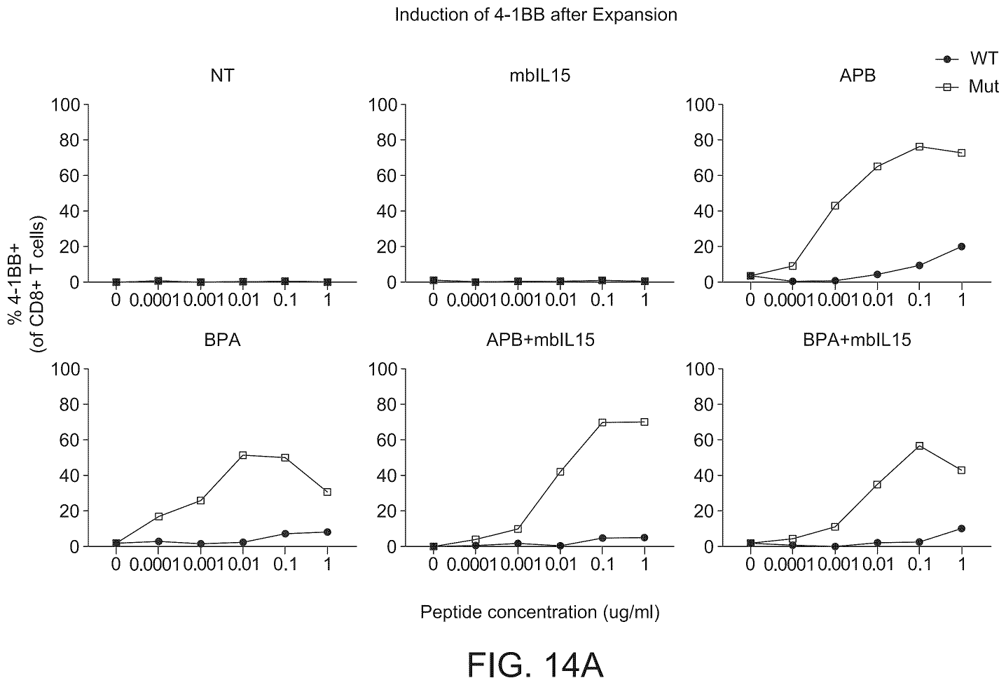

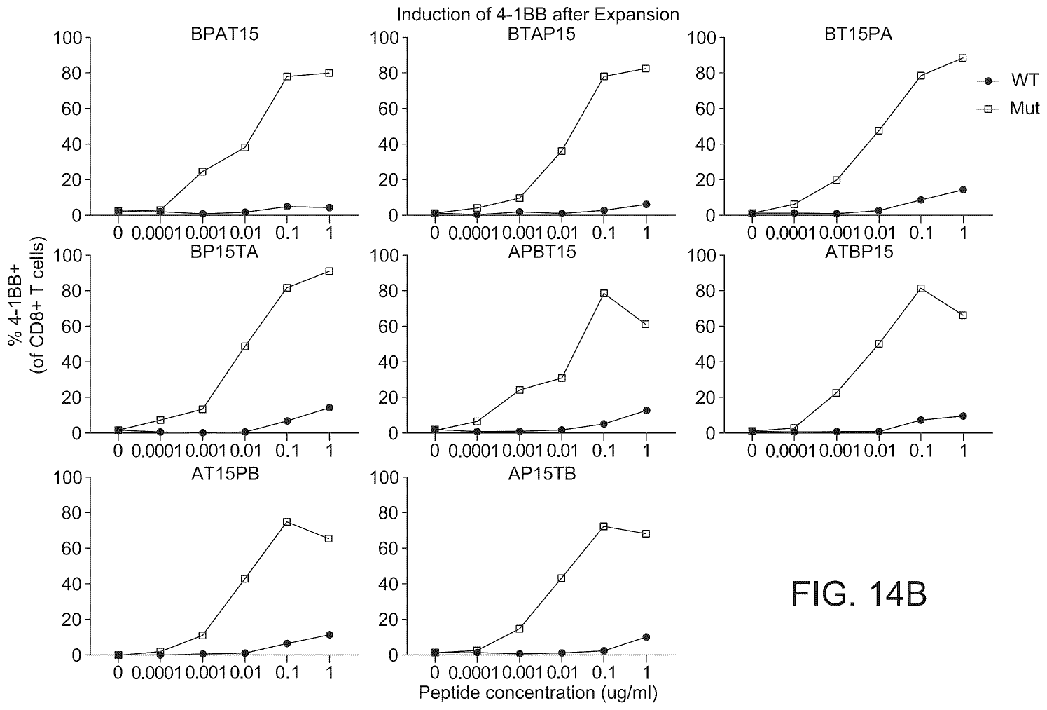

Activation of TCR-T cells generated by electroporation with different polycistronic plasmids was assessed. After 13 days of first phase expansion, cells were co-cultured with wild-type or mutant neoantigen peptide pulsed T2 cells. After overnight incubation, cells were harvested and induction of 4-1BB molecule on CD3+CD8+ cells was detected with 4-1BB antibody. Results are shown in FIG. 14A-14B demonstrating that mbIL15/TCR T cells were highly avid and specific to the target neoantigen as measured by upregulation of 4-1BB co-stimulatory receptor with negligible recognition of wild type sequence. There was no significant difference in function between the mbIL15/TCR T cells generated using different polycistronic plasmids.

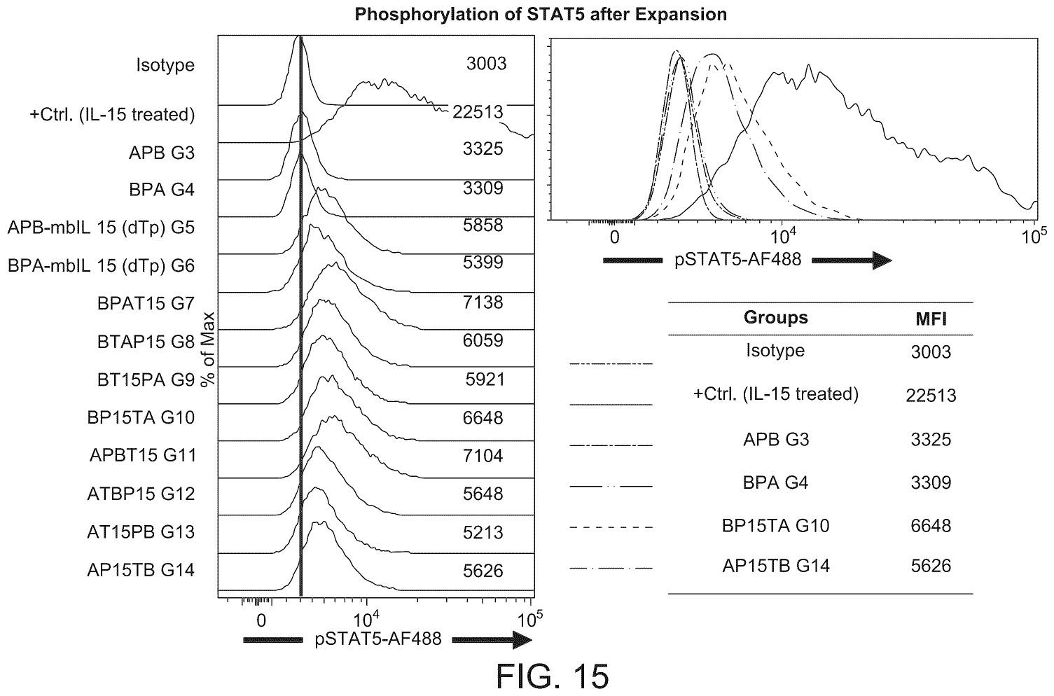

The level of phosphorylated STAT5 was also assessed for TCR-T cells electroporated with different polycistronic plasmids. After 13 days of first phase expansion, cells were washed and incubated in cytokine-free 50:50 media overnight to stabilize the phosphorylation of STAT5. Phosphorylation of STAT5 was detected the following day on CD3+ cells using pSTAT5 (pY694). Results are shown in FIG. 15 demonstrating that the expressed mbIL15 is functional. IL15 signaling was activated, inducing phosphorylation of STAT5 (downstream of IL15 receptor). Phosphorylation of STAT5 in mbIL15 TCR T cells generated with different polycistronic plasmids was not significantly different.

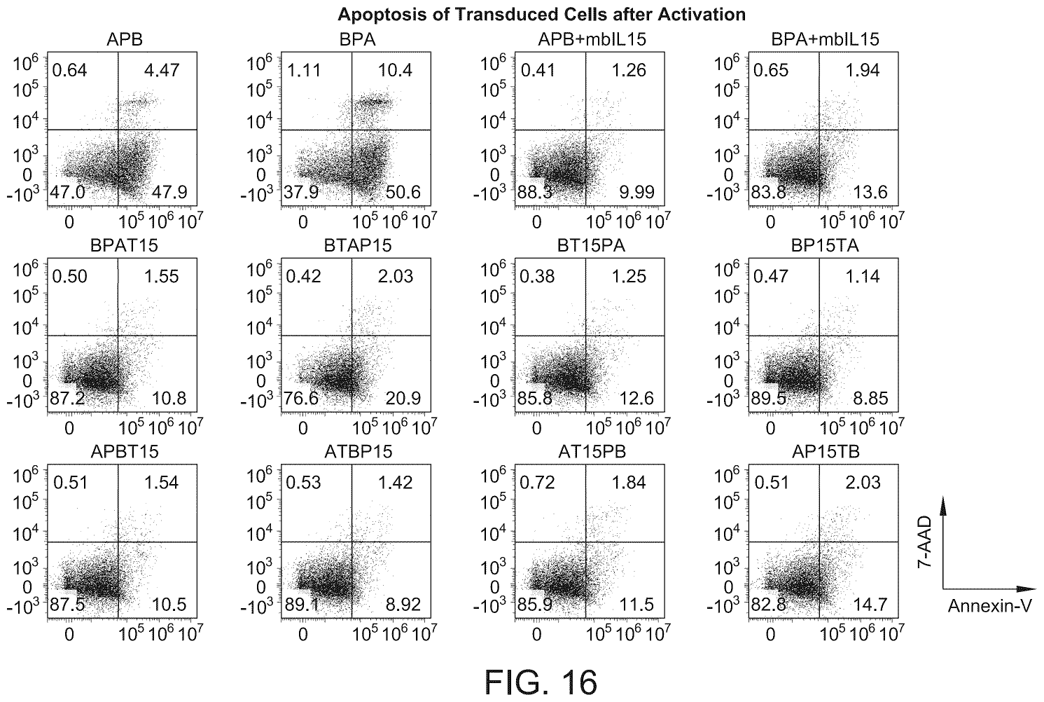

The level of apoptosis after 9 days of activation was assessed for TCR-T cells electroporated with different polycistronic plasmids. After 13 days of first phase expansion, cells were washed and activated with CD3/CD28 Dynabeads® (ThermoFisher) for 9 days. After activation, apoptosis of CD3+ TCR+ cells was monitored with Annexin V kits (Biolegend) and Live/Dead stain (Invitrogen). Results are shown in FIG. 16 demonstrating that expression of mbIL15 on CD3+ TCR+ cells inhibited AICD (activation-induced cell death). This inhibition of AICD was not significantly different between the different polycistronic plasmids tested, nor was it different from two-vector systems (APB+mbIL15 and BPA+mbIL15).

A second expansion phase was performed as described below and vector copy number (VCN) following the second expansion phase was assessed. Briefly, T cells from Groups 3-14 were isolated by MACS using mTCR antibodies. T cells from Groups 1-14 were then incubated with a second expansion media (50:50 media containing IL-21) and irradiated feeder cells and OKT3 antibody. Cells were fed regularly with cytokines. After 15 days second phase expansion, cells were harvested and VCN was detected using qPCR as average number of Sleeping Beauty transgene DNA copy per cell in a sample. Results are shown in Table E9 demonstrating that low levels of vector were detected in TCR T cells and mbIL15-TCR T cells after two rounds of expansion.

TABLE E9
Vector Copy Number (VCN) after second expansion phase.

| Group | Name | VCN |
|---|---|---|
| 2 | mbIL15 cells | 0.3 |
| 3 | APB cells | 0.4 |
| 4 | BPA cells | 1.4 |
| 5 | APB+15 cells | 2.6 |
| 6 | BPA+15 cells | 2.1 |
| 7 | APBT15 cells | 4.7 |
| 8 | ATBP15 cells | 4.5 |
| 9 | AP15TB cells | 5.3 |
| 10 | AT15PB cells | 3.9 |
| 11 | BPAT15 cells | 1.5 |
| 12 | BTAP15 cells | 2.1 |
| 13 | BP15TA cells | 2.3 |
| 14 | BT15PA cells | 2.9 |

Conclusion: The series of data described in this example illustrate that BP15TA and AP15 TB are the most potent candidates to generate mbIL15 TCR T cell with the highest level of TCR and mbIL15 expression. All tricistronic mbIL15/TCR plasmids tested resulted in acceptable VCN values. Furthermore, co-expression of mbIL15 with a transgenic TCR, reduces AICD following T cell activation.

### Example 5: Evaluation of Polycistronic TCR Constructs with Different Murine Constant Regions

This Example evaluates the effect of different murine constant regions on the TCR constructs described above in Examples 1-4.

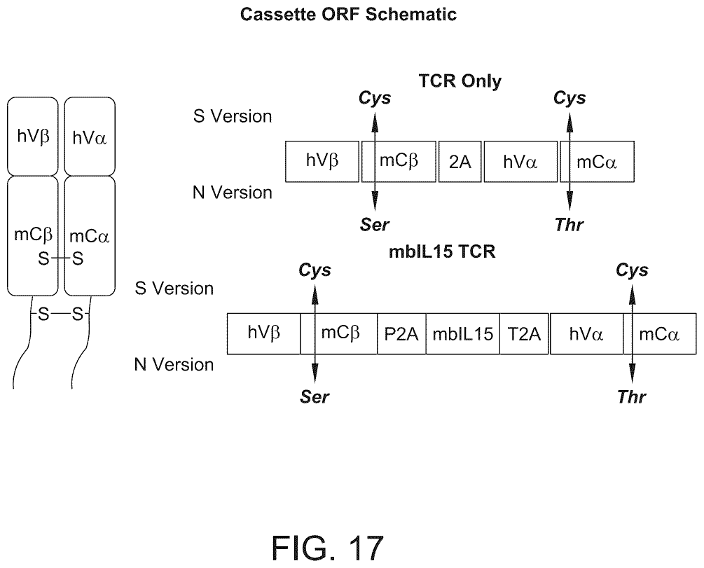

Briefly, the amino acid sequences of the TCRα chain and TCRβ chain examined here are identical to the TCRα chain and TCRβ chain described in Examples 1-4 except that the constant region of each chain is not cysteine-substituted. Specifically, the TCRα chain was generated by fusing a human Vα region, including its N-terminal signal sequence (SEQ ID NO: 1006) with a glutamic acid at position 2, to a murine Cα region modified by substituting a leucine at amino acid position 112, an isoleucine at amino acid position 114, and a valine at amino acid position 115 (SEQ ID NO: 42). The TCRβ chain was generated by fusing a human Vβ region, including its N-terminal signal sequence (SEQ ID NO: 2006) with an alanine at position 2, to a murine wild-type Cβ (SEQ ID NO: 52). The constructs containing the cysteine-substituted constant domains, as described in Examples 1-3, are referred to below as the “S version” and the newly-generated constructs containing the non-cysteine-substituted constant domains are referred to below as the “N version”. A schematic of these constructs is provided in FIG. 17.

The unified plasmids “NU version” referred to below vary in the nucleotide sequence of the TCR constant regions compared to the “N version”. All “NU versions” contain the same nucleotide sequences encoding the TCR constant regions. However, the amino acid sequences of the TCR constant regions encoded by the “NU version” are identical to those of the “N version”. No other differences exist between the “N version” and “NU version.”

To generate the TCR-T cells described in this Example 5, the plasmids described above were electroporated into the enriched PBMCs. Briefly, cryopreserved PBMCs were thawed, resuspended in supplemented media, and incubated in a 37° C./5% CO₂ incubator for one hour. The PBMC test articles listed in Table E10 were then prepared.

TABLE E10
PBMC test articles.

| Group | Name | Description |
|---|---|---|
| 2.1 | NT cells | Non-transposed PBMCs |
| 2.2 | BPA cells | PBMCs transposed with Plasmid BPA |
| 2.3 | BPA-N cells | PBMCs transposed with N version of Plasmid BPA |
| 2.4 | AP15TB cells | PBMCs transposed with Plasmid AP15TB |
| 2.5 | BP15TA cells | PBMCs transposed with Plasmid BP15TA |
| 2.6 | AP15TB-N cells | PBMCs transposed with N version of Plasmid AP15TB |
| 2.7 | BP15TA-N cells | PBMCs transposed with N version of Plasmid BP15TA |
| 2.8 | AP15TB-NU cells | PBMCs transposed with unified N version of Plasmid AP15TB |
| 2.9 | BP15TA-NU cells | PBMCs transposed with unified N version of Plasmid BP15TB |

Test articles were prepared as follows:

Group 2.1: Rested cells were harvested, spun down, resuspended in recovery media (50:50 media containing IL-7+IL-15+n-acetylcysteine (NAC)), and incubated in a 37° C./5% CO₂ incubator overnight.

Groups 2.2-2.9: Rested cells were harvested, spun down, resuspended in electroporation buffer together with the plasmids listed in Table E10, and electroporated. Following electroporation, cell suspensions were collected, transferred to recovery media (50:50 media containing IL-7+IL-15+NAC), and incubated in a 37° C./5% CO₂ incubator overnight.

Within 24 hours post-electroporation (Day 1), live cells were transferred to G-REX® culture plates (Wilson Wolf Manufacturing) and incubated with a first expansion media (50:50 media containing IL-21+IL-7+IL-12 and T Cell TransACT™). Cells were regularly fed with cytokines. After 11 days of first phase expansion, TCR+ cells were isolated with mTCR antibody. The isolated TCR+ T cells were transferred to G-REX® culture plates (Wilson Wolf Manufacturing) and incubated with a second expansion media (50:50 media containing 3000 IU/ml of IL-2 and T Cell TransACT™). Cells were fed regularly with cytokines. After 11 days of second phase expansion, cells were harvested, and the various assays described below were performed.

Transgene expression was assessed for T cells electroporated with different polycistronic plasmids. On Day 1 (post-transduction), Day 11 (post-1^{st} phase expansion) and Day 22 (post-2^{nd} phase expansion), cells were harvested and the expression of mTCR and mbIL15 was detected on CD3+ gated population with mouse TCR beta antibody and IL-15Rα antibody.

Fold expansion of total cell count and mTCR+ cell count was assessed for T cells electroporated with different polycistronic plasmids. Fold expansion value was calculated as: Cell number on Day 11/Cell number on Day 1 and Cell number on Day 22/Cell number on Day 11. Cells transposed with mbIL15/TCR tricistronic plasmids tended to expand less than cells transposed with TCR only bicistronic plasmids during both first and second phase expansion. However, significant degrees of expansion were achieved in all groups and no difference was seen between the different versions of the polycistronic plasmids. mTCR+ cell number was calculated as: Total cell number×CD3 population (%)×mTCR population (%).

The above transgene expression and cell growth data demonstrate that cells generated using N version and NU versions of the polycistronic plasmids were not phenotypically different from cells generated using the S version of the polycistronic plasmids.

Memory phenotype was assessed by multicolor flow cytometry for TCR-T cells electroporated with different polycistronic vectors. 2×10⁵ live TCR-T cells were used for multicolor flow cytometry assay. Graphs are sets of 2-parameter flow dot plots showing the expression of the T cell markers CD62L and CD45RO from one Donor at day 22 post-expansion. T cell memory subsets are defined as: CD62L+CD45RO+=central memory (Tcm); CD62L-CD45RO+=effector memory (Tem). Memory phenotype data at day 22 demonstrated that memory phenotype is not different between TCR only groups and mbIL15 TCR groups and is not different depending on which version of vector is used (S version vs. N version or NU version).

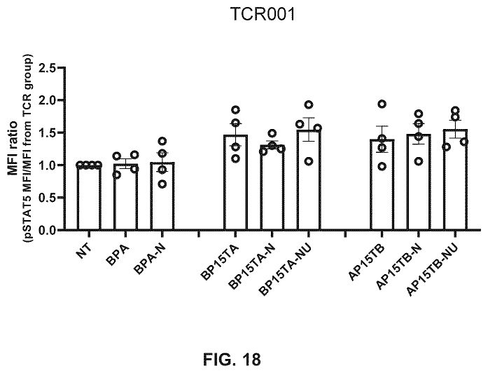

To carry out the pSTAT5 assay, the 4-1BB induction assay, and IFN-γ assay described below, the second expansion phase was extended to 16 days (due to the logistic load). Phosphorylation of STAT5 in T cells at Day 27 was detected on CD3+ cells with pSTAT5 (pY694). The pSTAT5 data shown in FIG. 18 demonstrated that the expressed mbIL15 is functional. IL15 signaling was activated, inducing phosphorylation of STAT5 (downstream of IL15 receptor). Phosphorylation of STAT5 in mbIL15 TCR-T cells generated with the different versions of polycistronic plasmids was not significantly different.

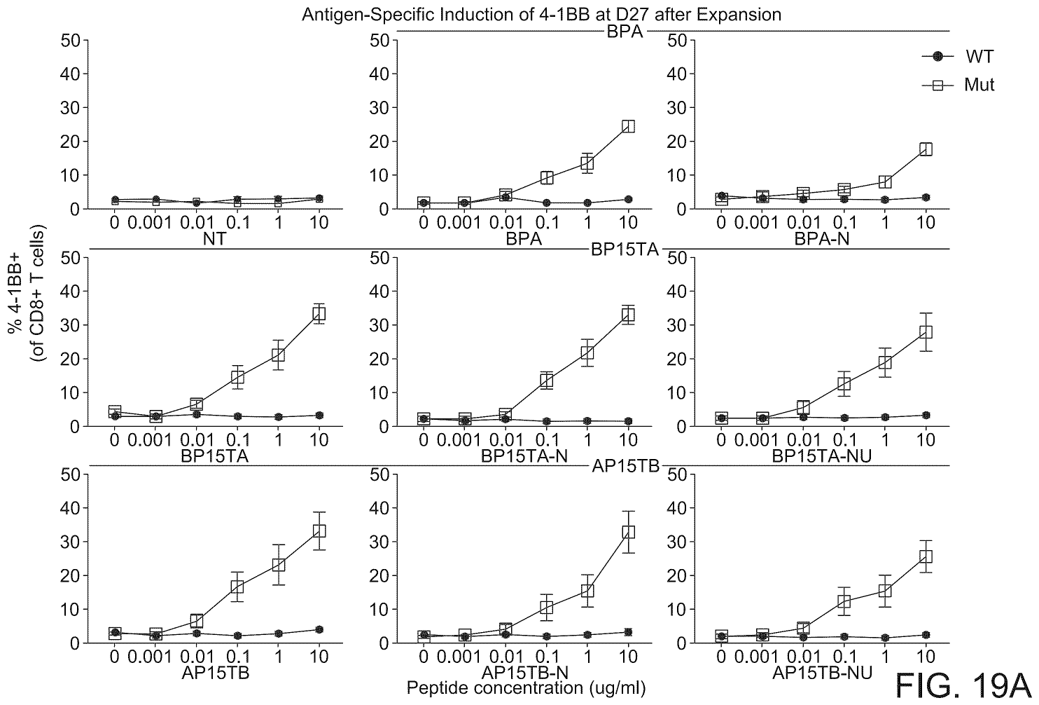

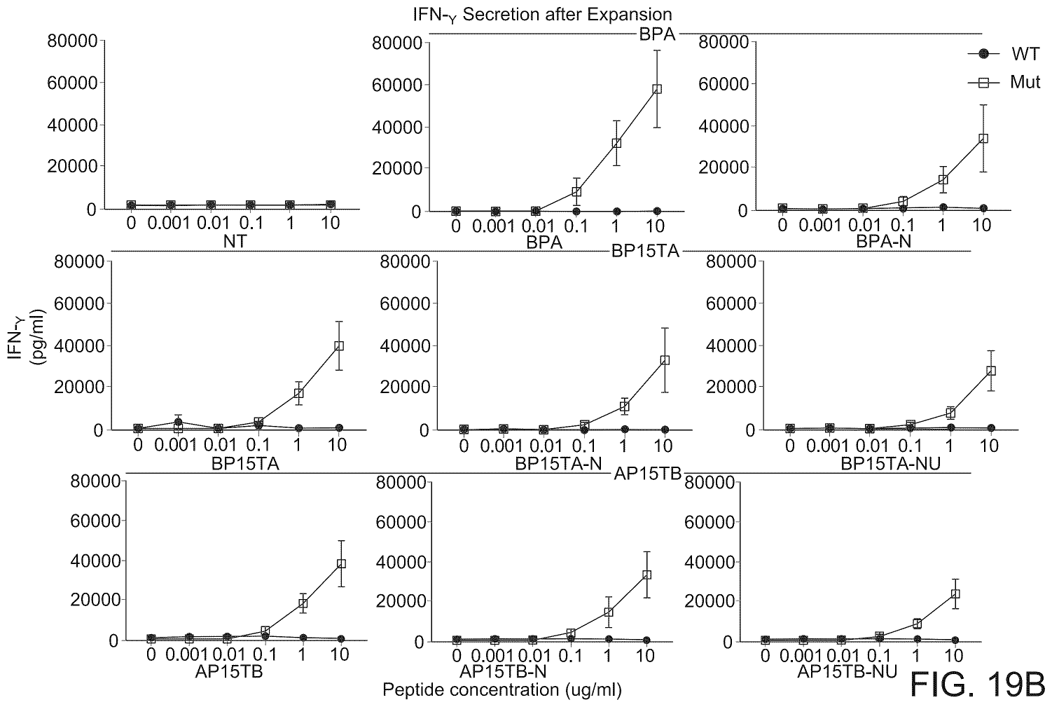

To assess activation of the generated TCR-T cells, overnight co-culture of the generated TCR-T cells with wild-type or mutant neoantigen pulsed DCs (HLA matched) was performed after 16 days second phase expansion and 4-1BB induction and IFN-γ secretion were measured. Induction of 4-1BB on CD3+CD8+ cells was detected with 4-1BB antibody. Secretion of IFN-γ measured with the ELISA antibody pair. The 4-1BB induction results shown in FIG. 19A, and IFN-γ secretion results shown in FIG. 19B demonstrate that the function of mbIL15 TCR T-cells generated with different versions of the polycistronic plasmids was not significantly different.

The long-term withdrawal (LTWD) assay was performed to examine the transgene expression, survival and activation of T cells cultured under cytokine-free conditions. The LTWD assay was performed as follows. The engineered T cells at Day 22 (post first phase and second phase expansion) were transferred to T25 flask and cultured for 4 weeks in cytokine-free media (50:50). 50% of media was exchanged every week. For the control groups (groups 2.2 & 2.3), cells were treated with 300 U/ml IL-2 twice a week while exchanging the 50% of media. Flow data were acquired using an NovoCyte Quanteon flow cytometer system (Agilent) and analyzed with FlowJo software (version 10.7.1; TreeStar, Ashland, OR). (Data n=4, pooled from 2 independent experiments)

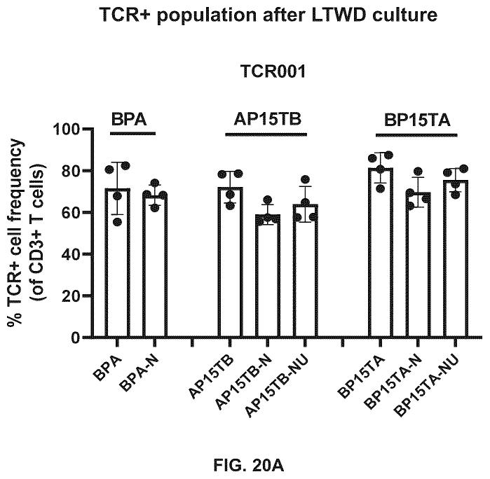

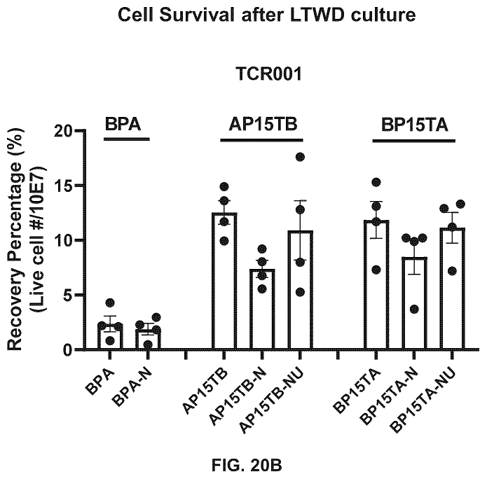

After 4 weeks LTWD incubation, the expression of mTCR was detected on CD3+ gated population with mouse TCR beta antibody (FIG. 20A) and cell count and viability were accessed (FIG. 20B). This mTCR expression and cell count data demonstrated no significant difference between mbIL15 TCR-T cells generated with different versions of the polycistronic plasmids. The number of viable cells decreased after long-term cytokine withdrawal in all groups, but cells from the groups co-expressing mbIL15 and TCR survived 5-6 fold more compared to cells from the TCR only groups.

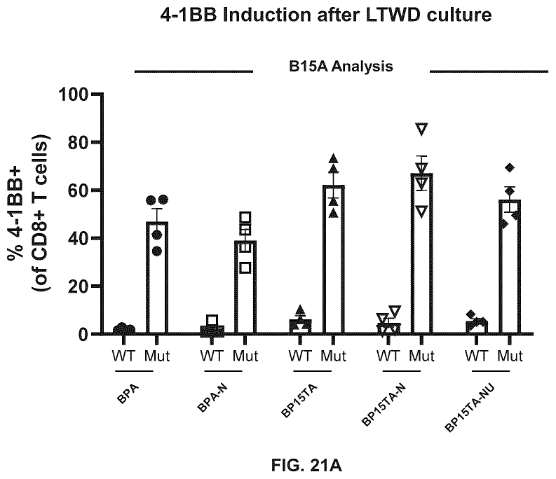

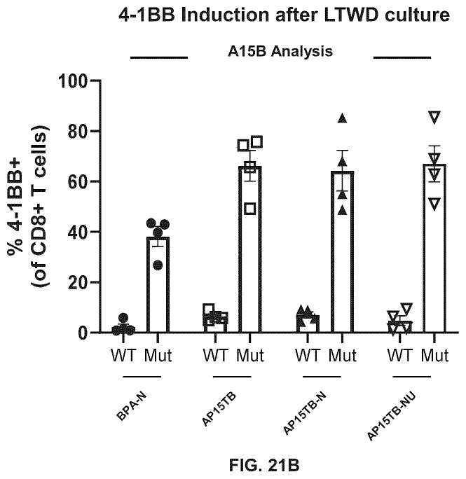

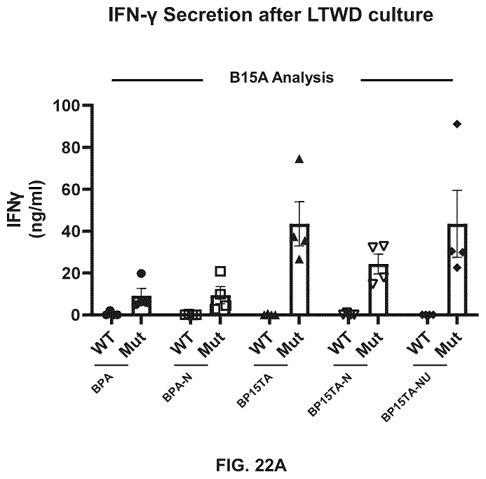

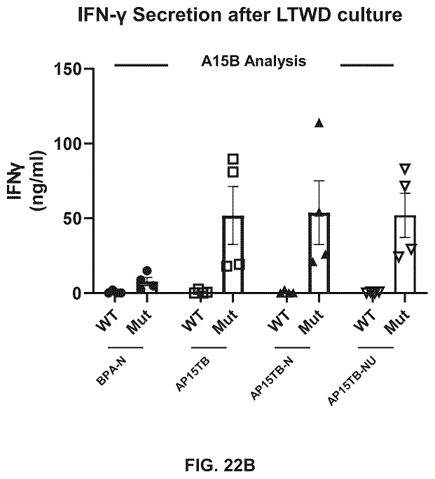

The activation of TCR-T cells after LTWD culture was assessed by 4-1BB induction and IFN-γ secretion after overnight co-culture with wild-type or mutant neoantigen (10 μg/ml) pulsed DCs (HLA matched). As described above, induction of 4-1BB on CD3+CD8+ cells was detected with 4-1BB antibody (FIG. 21A-21B) and IFN-7 secretion was measured with the ELISA antibody pair (FIG. 22A-22B). These data demonstrate that mbIL15 TCR-T cells which survived LTWD culture are still functional and were more strongly activated than cells from TCR only groups, but the function of mbIL15 TCR T-cells generated with different versions of the polycistronic plasmids was not significantly different.

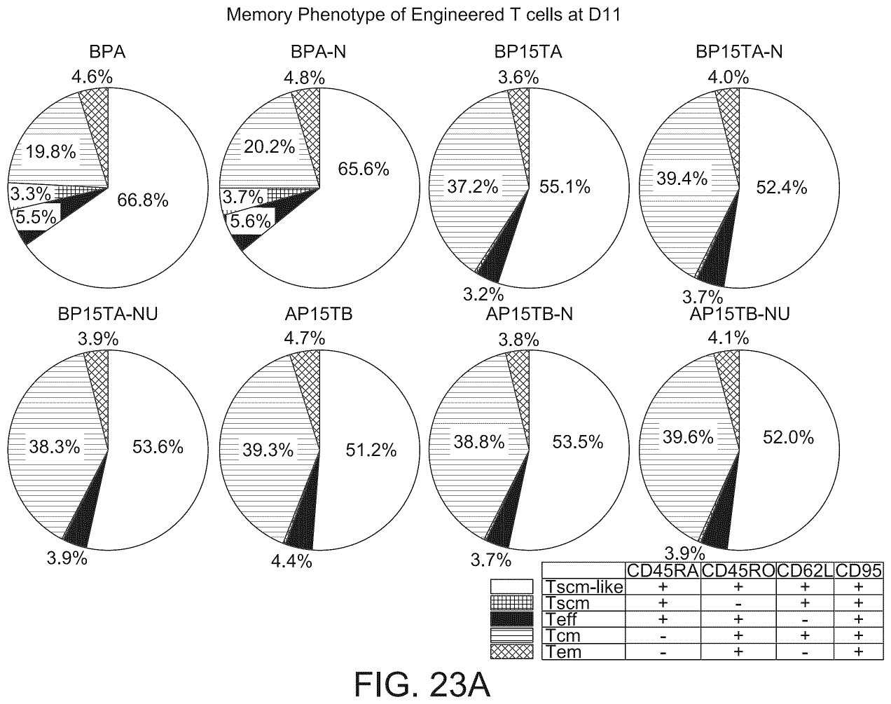

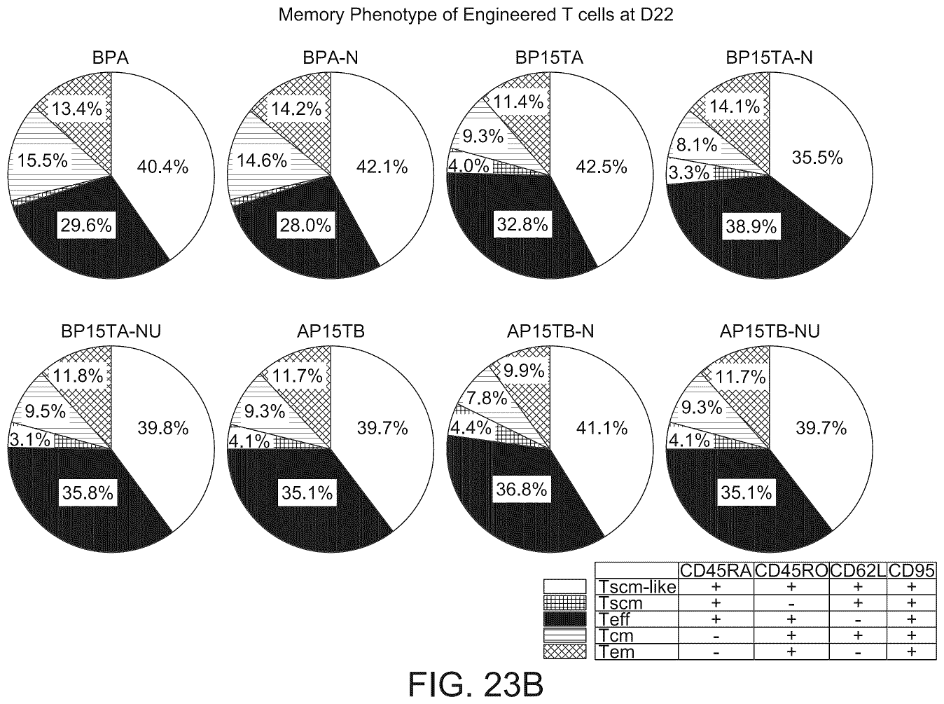

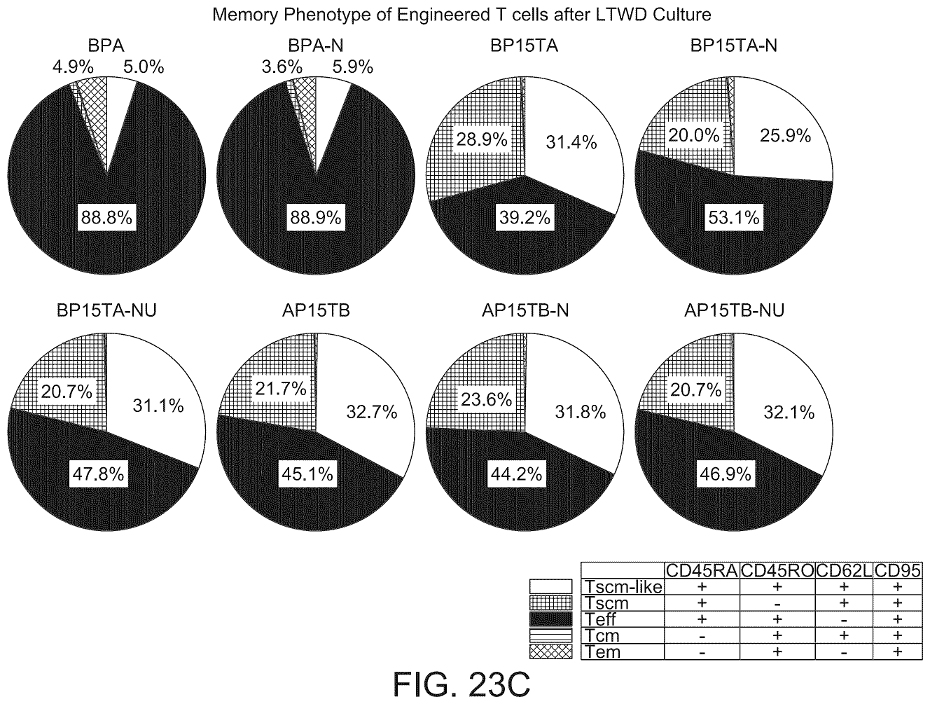

Memory phenotype of TCR-T cells electroporated with different polycistronic vectors was also assessed. T cell memory subsets are defined as: CD45RA+CD45RO+CD62L+CD95+=stem cell memory-like (Tscm-like); CD45RA+CD45RO-CD62L+CD95+=stem cell memory (Tscm); CD45RA-CD45RO+CD62L+CD95+=central memory (Tcm); CD45RA-CD45RO+CD62L-CD95+=effector memory (Tem). T cell effector (Teff) are defined as CD45RA+CD45RO+CD62L-CD95+. The pie charts in FIG. 23A-23C show the mean frequency of live CD3⁺ T cell memory and effector subsets at day 11 post-expansion (FIG. 23A), day 22 post-expansion (FIG. 23B), and after 4 weeks of LTWD culture (FIG. 23C) in cells transposed with the tested plasmids.

Memory phenotype data shows the kinetics of TCR-T memory and effector differentiation. At days 11 and 22 post-expansion, there is no difference between the different polycistronic TCR plasmids. After 4 weeks of culture in presence of IL-2, TCR-T cells predominantly differentiated into Teff cells (over 85%). TCR-T cells expressing mbIL15 cultured for 4 weeks in the absence of cytokines differentiated into 3 main subsets: Teff, Tscm-like and Tscm cells. These results suggest that mbIL15 is sufficient to guide T cell differentiation to the Tscm phenotype.

Conclusions: The mbIL15 T cells generated from different versions of the polycistronic plasmids showed comparable features including TCR expression, memory phenotype, specificity, and IFN-γ secretion. This data supports that removal of cysteine-substitutions in the mouse constant domains used in the first-generation vectors and use of unified mouse constant regions will not produce any significant changes in the mbIL15 TCR T-cell product.

### Example 6: Generation and Evaluation of T Cells Generated Using Various Tricistronic TCR/mbIL15 Vectors

This Example describes the evaluation of T cells expressing mbIL15 in combination with different TCRα/TCRβ chains generated using tricistronic vectors as described below. Similar to the vectors described in Example 5, the tricistronic expression cassettes used in this Example each include a transcriptional regulatory element operably linked to a polycistronic polynucleotide that encodes a TCR α chain (referred to herein as “TCRα” or “A”), a TCR β chain (referred to herein as “TCRβ” or “B”), and membrane-bound IL-15/IL-15Rα fusion protein (referred to herein as “mbIL15” or “15”), each separated by a furin recognition site and either a P2A element or a T2A element that mediates ribosome skipping to enable expression of separate polypeptide chains.

The nine TCRs used in this Example are each directed against a different target as shown in Table E11. The Vα amino acid sequences and Vβ amino acid sequences for each of the nine TCRs listed correspond to the sequences provided in Table 6. Each TCR α chain was generated by fusing the Vα sequence to a murine Cα region modified by substituting a leucine at amino acid position 112, an isoleucine at amino acid position 114, and a valine at amino acid position 115 (SEQ ID NO: 42). Each TCRβ chain was generated by fusing the Vβ sequence to a murine wild-type Cβ (SEQ ID NO: 52).

TABLE E11
TCR Targets.

| Target Protein | Mutation | HLA Type | TCR |
|---|---|---|---|
| TP53 | R175H | A*02:01 | TCR001 |
| | | DRB1*13:01 | TCR009 |
| | R248W | A*68:01 | TCR057 |
| | Y220C | DRB3*02:02 | TCR016 |
| KRAS | G12D | A*11:01 | TCR022 |
| | | C*08:02 | TCR064 |
| | G12V | A*11:01 | TCR075 |
| | | C*01:02 | TCR055 |
| EGFR | E746-A750del | DPA1*02:01/DPB1*01:01 | TCR077 |

For each of the TCRs above, three vectors were constructed and evaluated: 1) TCR only (BA); 2) A15B; and 3) B15A. The TCR only (BA) vectors contain a bicistronic expression cassette encoding TCR β chain and TCR α chain separated by a furin recognition site and a P2A element in the following orientation from 5′ to 3′: TCRβ-TCRα. The A15B vectors contain a tricistronic expression cassette encoding TCR α chain, TCR β chain, and mbIL15 in the following orientation from 5′ to 3′: TCRα-mbIL15-TCRβ. The B15A vectors contain a tricistronic expression cassette encoding TCR α chain, TCR β chain, and mbIL15 in the following orientation from 5′ to 3′: TCRβ-mbIL15-TCRα.

TCR-T cells described in this Example were generated similarly to those described in Examples 2-5 except as indicated below.

Briefly, cryopreserved PBMCs were thawed, resuspended in supplemented media, and incubated in a 37° C./5% CO₂ incubator for one hour.

Test articles as listed in Table E12 were then prepared.

TABLE E12
PBMC test articles.

| Group | TCR | Name | Description |
|---|---|---|---|
| 3.1 | None | NT | Non-transposed PBMCs |
| 3.2 | TCR001 | BPA-N¹ | PBMCs transposed with TCR001 BPA-N |
| 3.3 | TCR001 | AP15TB-NU² | PBMCs transposed with TCR001 AP15TB-NU |
| 3.4 | TCR001 | BP15TA-NU³ | PBMCs transposed with TCR001 BP15TA-NU |
| 3.5 | TCR057 | BPA-N | PBMCs transposed with TCR057 BPA-N |
| 3.6 | TCR057 | AP15TB-NU | PBMCs transposed with TCR057 AP15TB-NU |
| 3.7 | TCR057 | BP15TA-NU | PBMCs transposed with TCR057 BP15TA-NU |
| 3.8 | TCR009 | BPA-N | PBMCs transposed with TCR009 BPA-N |
| 3.9 | TCR009 | AP15TB-NU | PBMCs transposed with TCR009 AP15TB-NU |
| 3.10 | TCR009 | BP15TA-NU | PBMCs transposed with TCR009 BP15TA-NU |
| 3.11 | TCR016 | BPA-N | PBMCs transposed with TCR016 BPA-N |
| 3.12 | TCR016 | AP15TB-NU | PBMCs transposed with TCR016 AP15TB-NU |
| 3.13 | TCR016 | BP15TA-NU | PBMCs transposed with TCR016 BP15TA-NU |
| 3.14 | None | NT | Non-transposed PBMCs |
| 3.15 | TCR022 | BPA-N | PBMCs transposed with TCR022 BPA-N |
| 3.16 | TCR022 | AP15TB-NU | PBMCs transposed with TCR022 AP15TB-NU |
| 3.17 | TCR022 | BP15TA-NU | PBMCs transposed with TCR022 BP15TA-NU |
| 3.18 | TCR075 | BPA-N | PBMCs transposed with TCR075 BPA-N |
| 3.19 | TCR075 | AP15TB-NU | PBMCs transposed with TCR075 AP15TB-NU |
| 3.20 | TCR075 | BP15TA-NU | PBMCs transposed with TCR075 BP15TA-NU |
| 3.21 | TCR055 | BPA-N | PBMCs transposed with TCR055 BPA-N |
| 3.22 | TCR055 | AP15TB-NU | PBMCs transposed with TCR055 AP15TB-NU |
| 3.23 | TCR055 | BP15TA-NU | PBMCs transposed with TCR055 BP15TA-NU |
| 3.24 | TCR064 | BPA-N | PBMCs transposed with TCR064 BPA-N |
| 3.25 | TCR064 | AP15TB-NU | PBMCs transposed with TCR064 AP15TB-NU |
| 3.26 | TCR064 | BP15TA-NU | PBMCs transposed with TCR064 BP15TA-NU |
| 3.27 | None | NT | Non-transposed PBMCs |
| 3.28 | TCR077 | BPA-N | PBMCs transposed with TCR077 BPA-N |
| 3.29 | TCR077 | AP15TB-NU | PBMCs transposed with TCR077 AP15TB-NU |
| 3.30 | TCR077 | BP15TA-NU | PBMCs transposed with TCR077 BP15TA-NU |

¹Generated using the same plasmid as BPA-N group in Example 5.
²Generated using the same plasmid as AP15TB-NU group in Example 5.
³Generated using the same plasmid as BP15TA-NU group in Example 5.

Test articles were prepared in three batches (Batch 1=Groups 3.1-3.13; Batch 2=Groups 3.14-3.26; Batch 3=Groups 3.27-3.30) as follows:

Groups 3.1 3.14, & 3.27: Rested cells were harvested, spun down, resuspended in recovery media (50:50 media containing IL-7+IL-15+n-acetylcysteine (NAC)), and incubated in a 37° C./5% CO₂ incubator overnight.

Groups 3.2-3.13, 3.15-3.26, & 3.28-3.30: Rested cells were harvested, spun down, resuspended in electroporation buffer together with the plasmids listed in Table E11, and electroporated. Following electroporation, cell suspensions were collected, transferred to recovery media (50:50 media containing IL-7+IL-15+NAC), and incubated in a 37° C./5% CO₂ incubator overnight.

Within 24 hours post-electroporation (Day 1), live cells were transferred to G-REX® culture plates (Wilson Wolf Manufacturing and incubated with a first expansion media (50:50 media containing IL-21+IL-7+IL-12 and T Cell TransACT™). Cells were regularly fed with cytokines. After 11 days of first phase expansion, TCR+ cells were isolated with mTCR antibody. The isolated TCR+ T cells were transferred to G-REX® culture plates (Wilson Wolf Manufacturing) and incubated with a second expansion media (50:50 media containing 3000 U/ml of IL-2 and T Cell TransACT™) During this second expansion phase, cells were regularly fed with cytokines. After 11 days of second phase expansion, cells were harvested, and the various assays described below were performed.

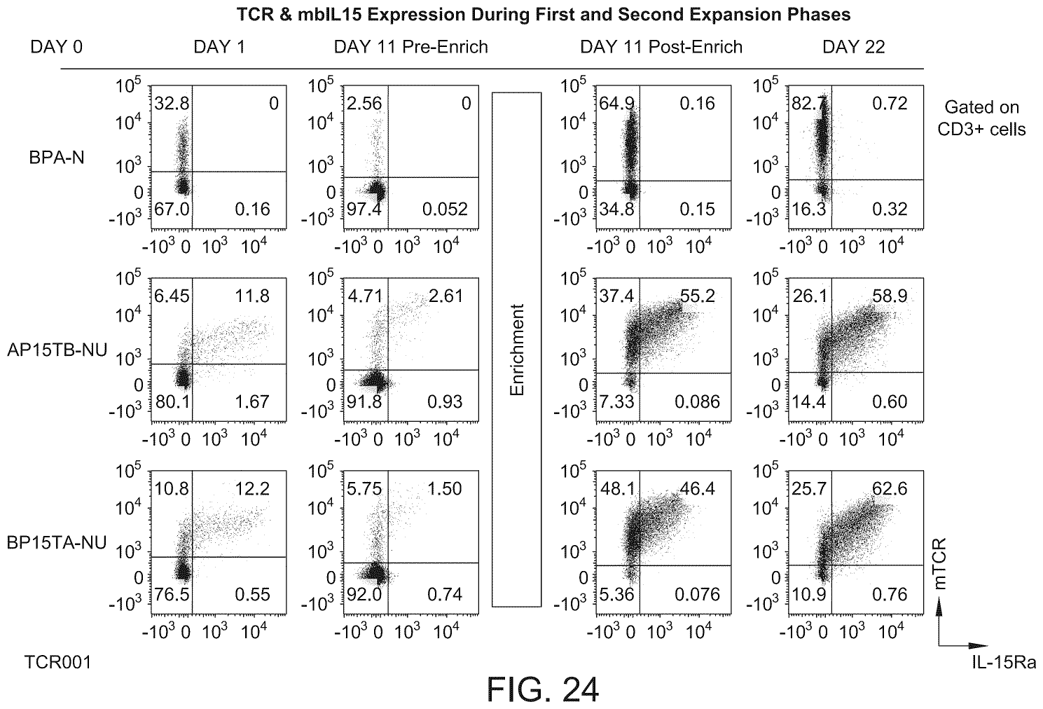

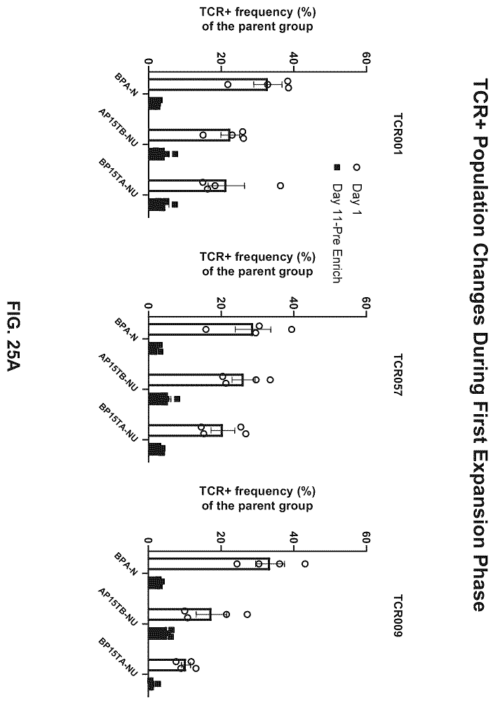

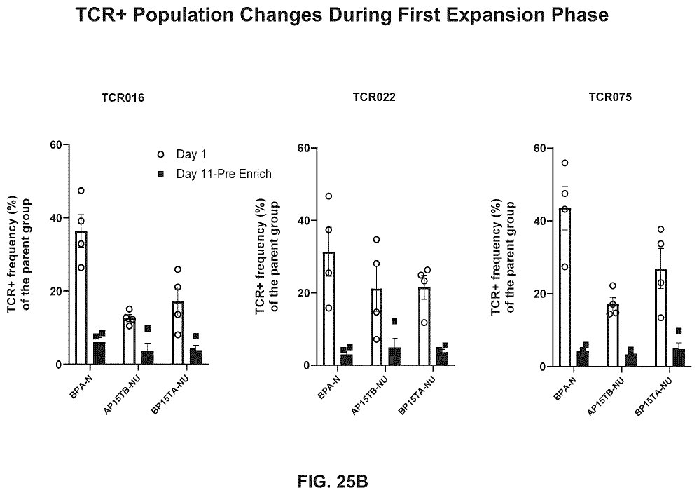

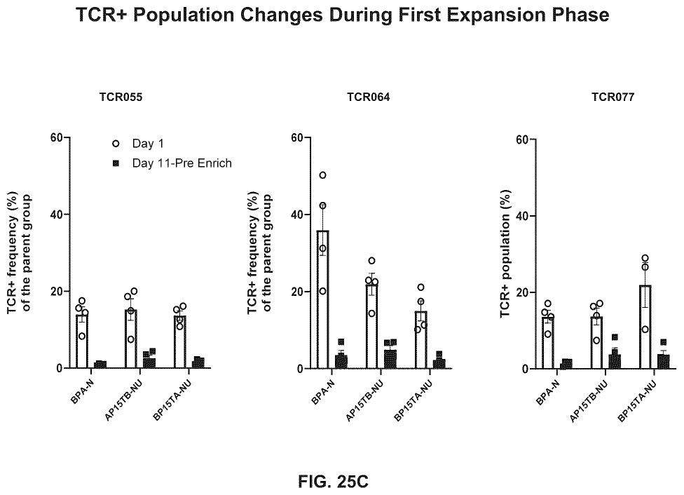

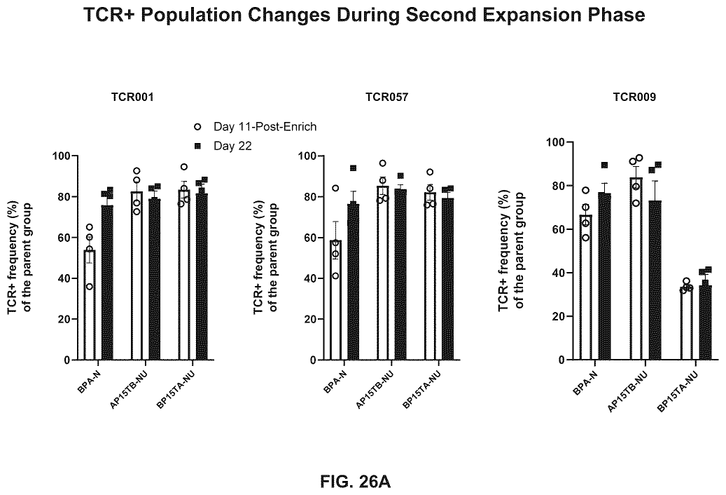

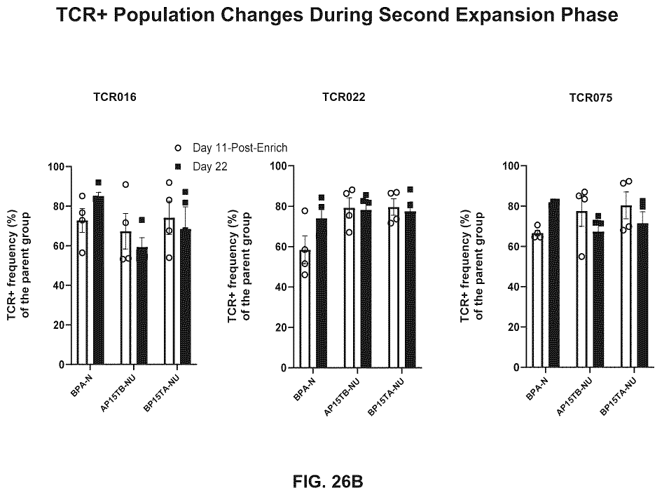

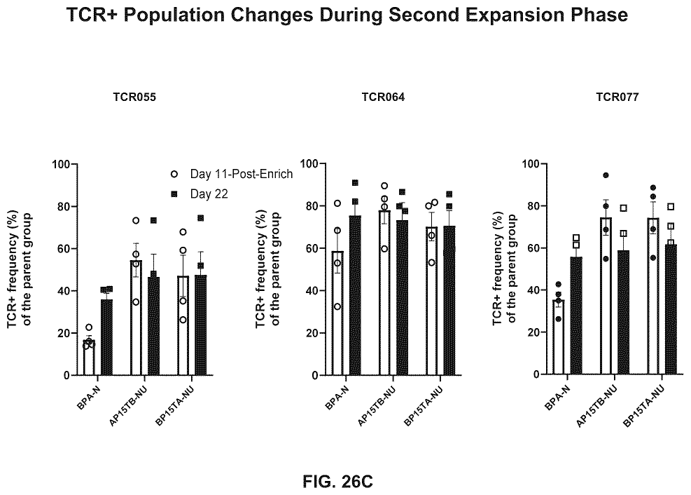

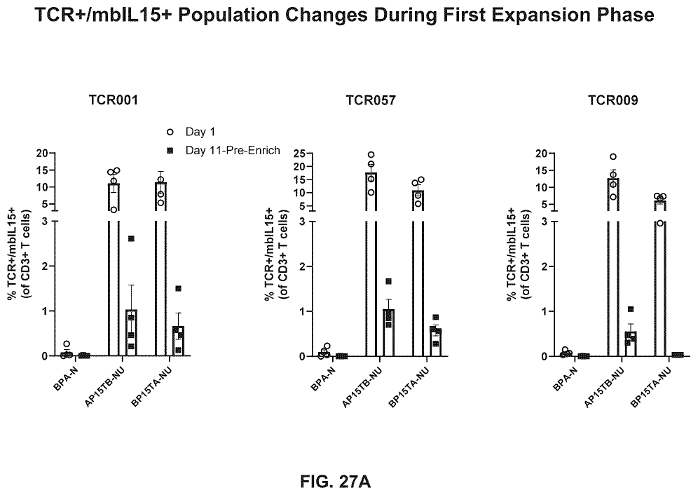

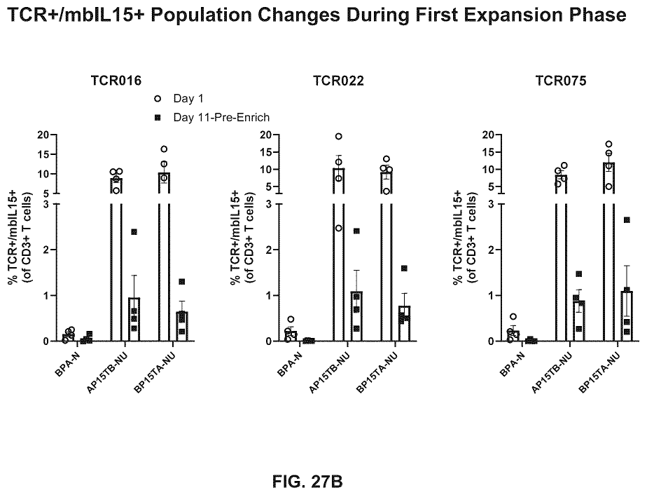

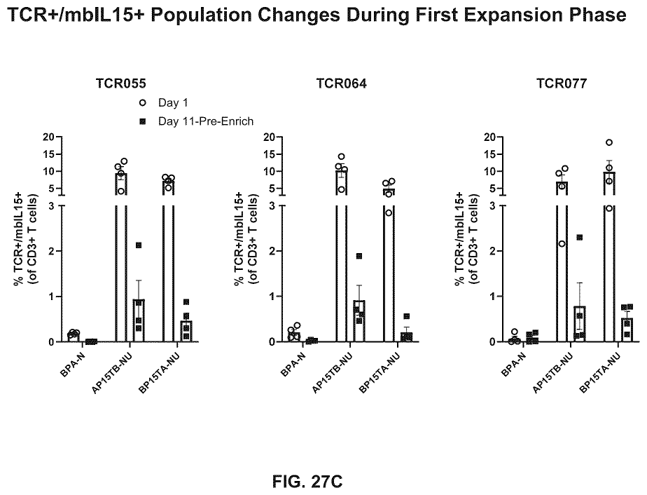

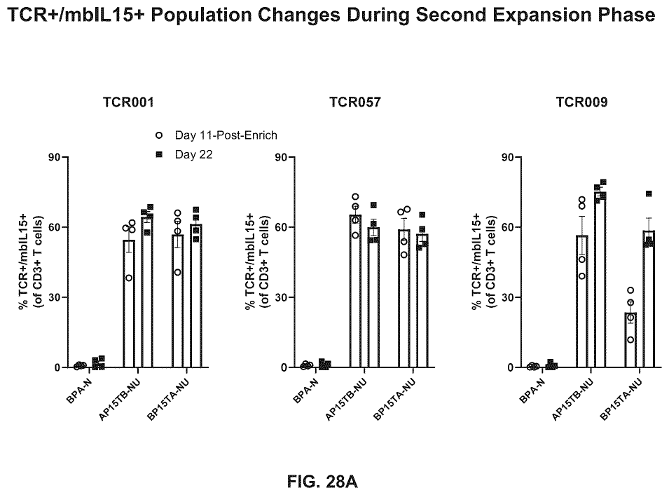

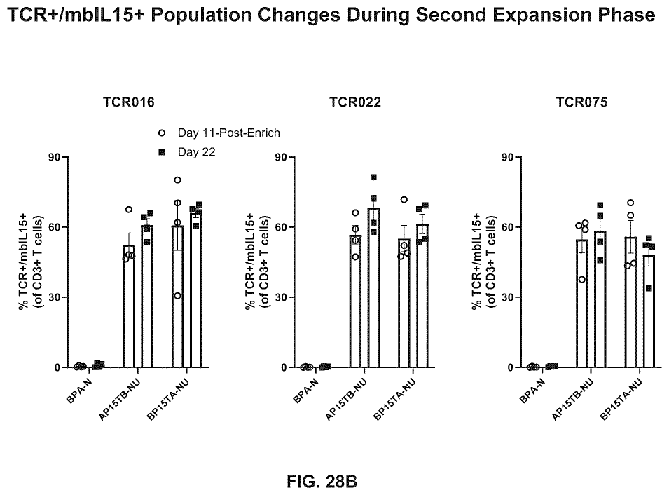

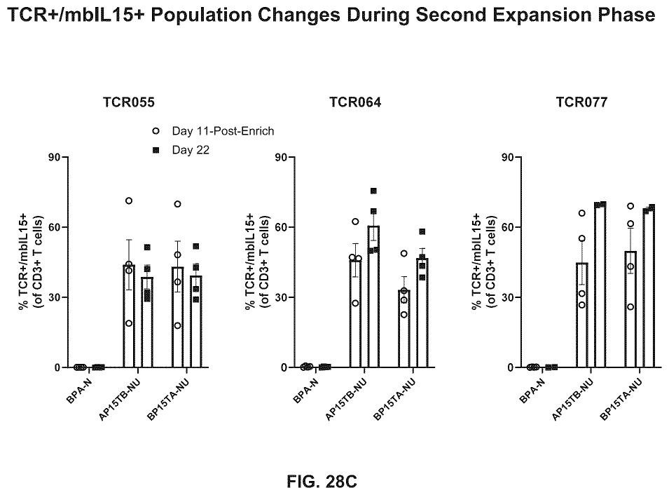

Transgene expression was assessed for T cells electroporated with different polycistronic plasmids. On Day 1 (post-transduction), Day 11 (post-1^{st} phase expansion) and Day 22 (post-2^{nd} phase expansion), cells were harvested and the expression of mTCR and mbIL15 was detected on CD3+ gated population with mouse TCR beta antibody and IL-15Rα antibody. The results are shown in FIGS. 24-28.

Fold expansion of total cell count and mTCR+ cell count was assessed for T cells electroporated with different polycistronic plasmids. Fold expansion value was calculated as: Cell number on Day 11/Cell number on Day 1 and Cell number on Day 22/Cell number on Day 11. mTCR+ cell number was calculated as: Total cell number×CD3 population (%)×mTCR population (%). The results are shown in Table E13.

TABLE E13
Fold expansion of total cells and mTCR+ cell
count during first and second expansion phases.

| | | Fold Expansion (FE) | | | | | | | |
|---|---|---|---|---|---|---|---|---|---|
| | | Total Cell | | | | mTCR+ Cell | | | |
| | | First | | Second | | First | | Second | |
| | | expansion | | expansion | | expansion | | expansion | |
| Group | | Mean | SEM | Mean | SEM | Mean | SEM | Mean | SEM |
| 1 | TCR001-BPA-N | 83.86 | 19.60 | 121.97 | 11.81 | 6.11 | 2.09 | 171.56 | 9.57 |
| | TCR001-AP15TB-NU | 66.89 | 13.09 | 107.01 | 20.38 | 18.38 | 9.60 | 101.00 | 17.14 |
| | TCR001-BP15TA-NU | 62.93 | 17.70 | 108.50 | 20.15 | 19.04 | 7.72 | 104.08 | 16.79 |
| 2 | TCR057-BPA-N | 98.21 | 36.03 | 110.40 | 7.05 | 8.16 | 4.73 | 146.17 | 13.45 |
| | TCR057-AP15TB-NU | 58.90 | 16.21 | 107.38 | 16.76 | 17.34 | 9.77 | 104.42 | 16.05 |
| | TCR057-BP15TA-NU | 58.78 | 16.21 | 108.69 | 3.91 | 14.19 | 6.44 | 104.04 | 3.64 |
| 3 | TCR009-BPA-N | 104.97 | 30.03 | 142.34 | 25.63 | 11.62 | 4.92 | 154.58 | 28.38 |
| | TCR009-AP15TB-NU | 51.68 | 13.48 | 95.38 | 13.31 | 24.76 | 12.75 | 83.83 | 16.19 |
| | TCR009-BP15TA-NU | 49.99 | 4.70 | 75.43 | 10.92 | 7.03 | 3.49 | 77.03 | 17.45 |
| 4 | TCR016-BPA-N | 87.13 | 9.63 | 127.79 | 25.67 | 17.38 | 5.29 | 142.91 | 26.78 |
| | TCR016-AP15TB-NU | 61.46 | 12.58 | 95.42 | 16.34 | 26.02 | 18.67 | 82.78 | 10.34 |
| | TCR016-BP15TA-NU | 66.33 | 15.42 | 93.38 | 14.09 | 23.39 | 15.51 | 83.30 | 14.44 |
| 5 | TCR022-BPA-N | 85.99 | 6.51 | 169.56 | 16.12 | 8.39 | 1.15 | 202.20 | 37.63 |
| | TCR022-AP15TB-NU | 59.11 | 5.90 | 95.36 | 12.47 | 12.21 | 1.59 | 102.12 | 22.10 |
| | TCR022-BP15TA-NU | 63.98 | 5.28 | 120.10 | 17.30 | 11.70 | 1.79 | 161.60 | 69.66 |
| 6 | TCR075-BPA-N | 89.45 | 6.14 | 182.86 | 15.27 | 9.12 | 0.75 | 207.74 | 21.54 |
| | TCR075-AP15TB-NU | 63.30 | 7.06 | 106.07 | 14.15 | 12.71 | 2.11 | 102.37 | 32.40 |
| | TCR075-BP15TA-NU | 59.06 | 5.91 | 107.40 | 20.65 | 10.57 | 2.47 | 114.25 | 43.15 |
| 7 | TCR055-BPA-N | 78.21 | 3.52 | 165.43 | 25.40 | 6.05 | 1.34 | 198.11 | 98.27 |
| | TCR055-AP15TB-NU | 58.84 | 1.25 | 95.98 | 18.09 | 10.28 | 1.79 | 67.59 | 20.05 |
| | TCR055-BP15TA-NU | 59.15 | 6.69 | 87.57 | 13.96 | 8.76 | 2.03 | 78.66 | 25.73 |
| 8 | TCR064-BPA-N | 86.10 | 10.54 | 177.48 | 15.01 | 8.47 | 1.48 | 212.18 | 55.16 |
| | TCR064-AP15TB-NU | 55.46 | 5.95 | 120.98 | 12.99 | 12.61 | 1.78 | 162.44 | 67.74 |
| | TCR064-BP15TA-NU | 59.66 | 4.38 | 110.54 | 16.75 | 9.78 | 1.98 | 137.63 | 59.08 |
| 9 | TCR077-BPA-N | 91.68 | 6.43 | 217.14 | 17.84 | 12.72 | 5.26 | 332.66 | 29.27 |
| | TCR077-AP15TB-NU | 57.10 | 5.54 | 97.22 | 13.45 | 16.66 | 7.28 | 75.79 | 4.98 |
| | TCR077-BP15TA-NU | 63.14 | 3.27 | 94.38 | 19.73 | 13.41 | 3.60 | 75.86 | 14.16 |

Cells generated using the polycistronic plasmids containing different TCR sequences were not phenotypically different from each other as demonstrated by transgene expression and cell growth data.

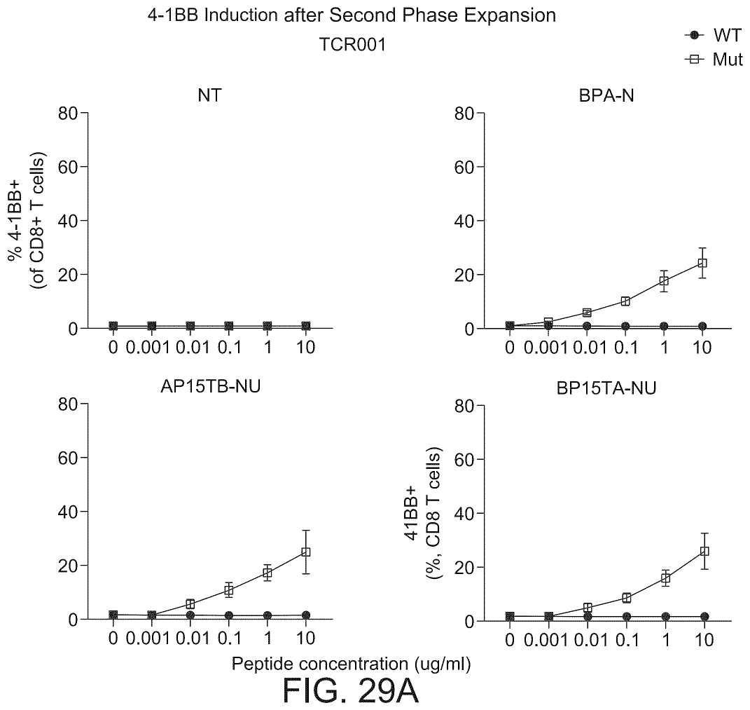

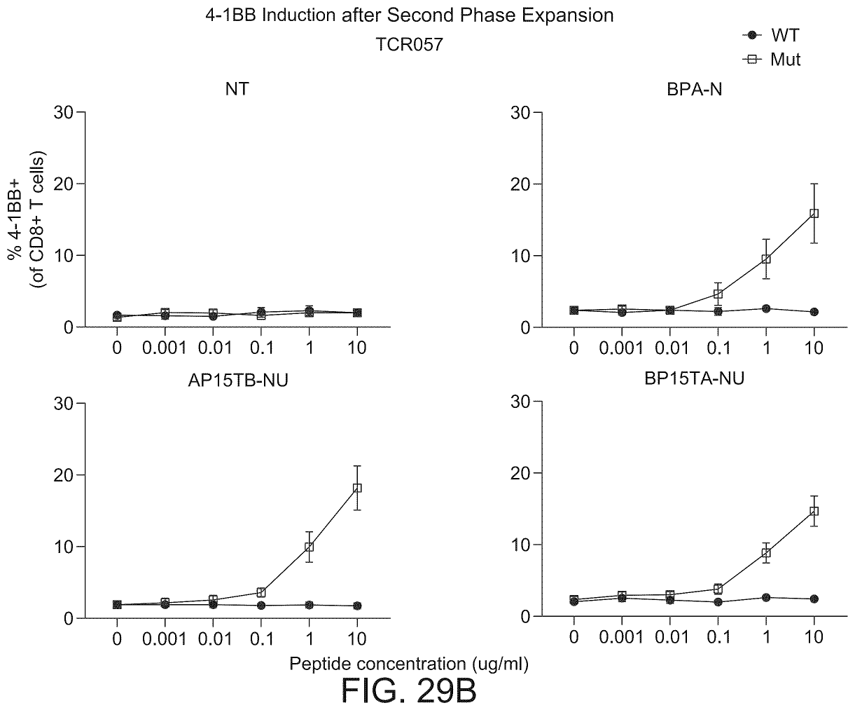

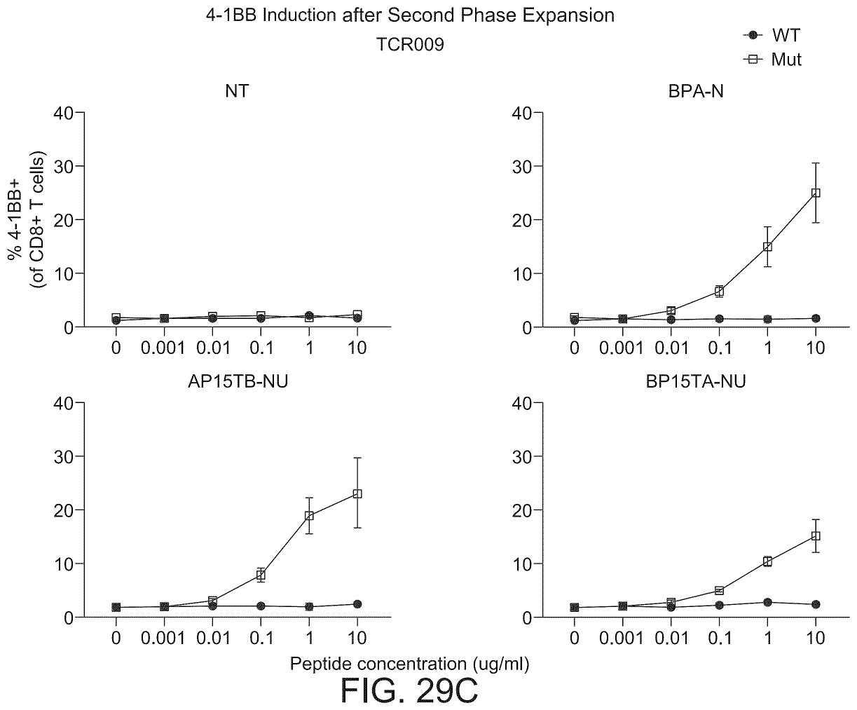

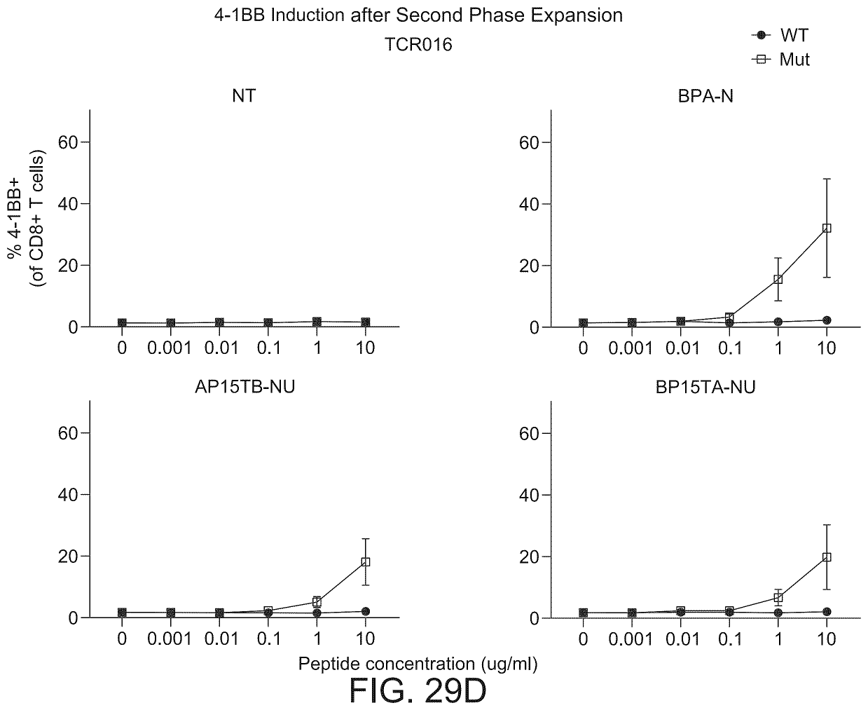

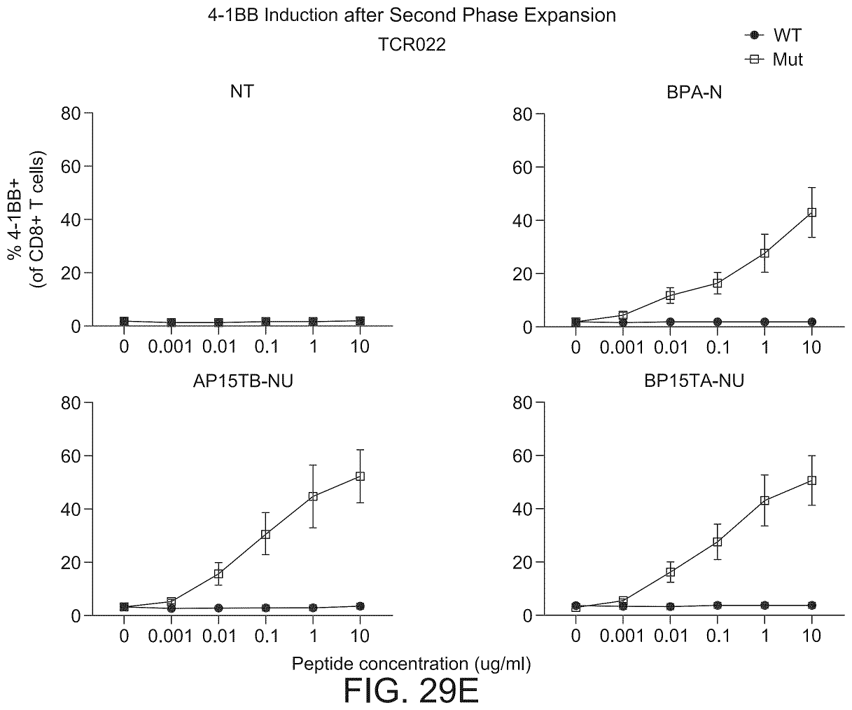

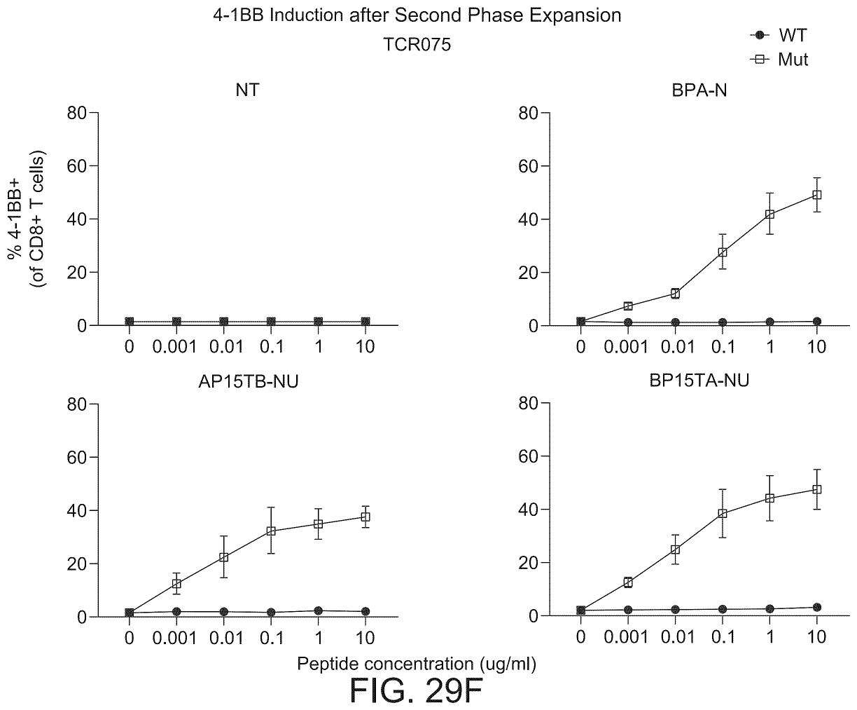

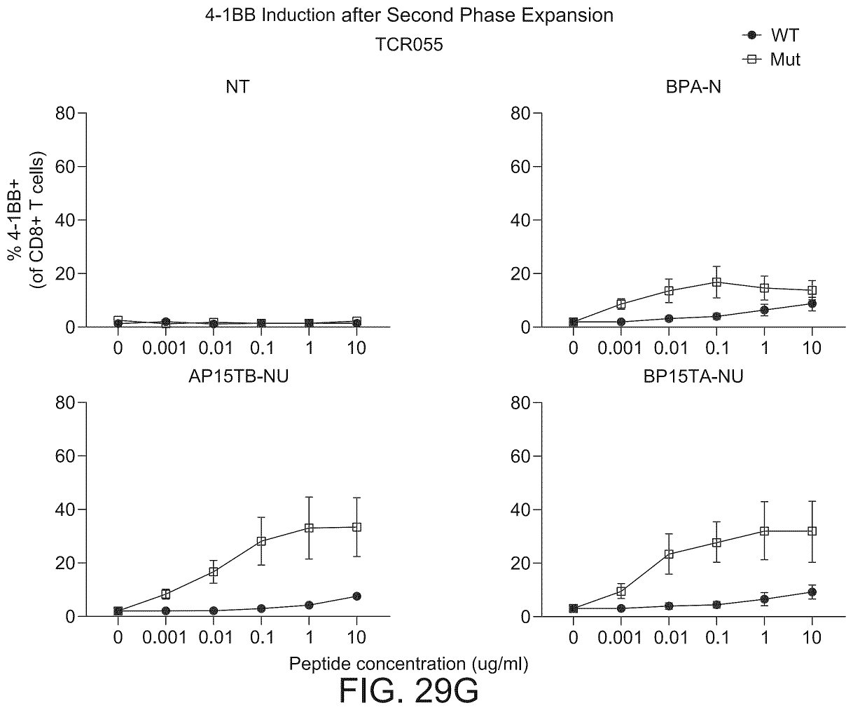

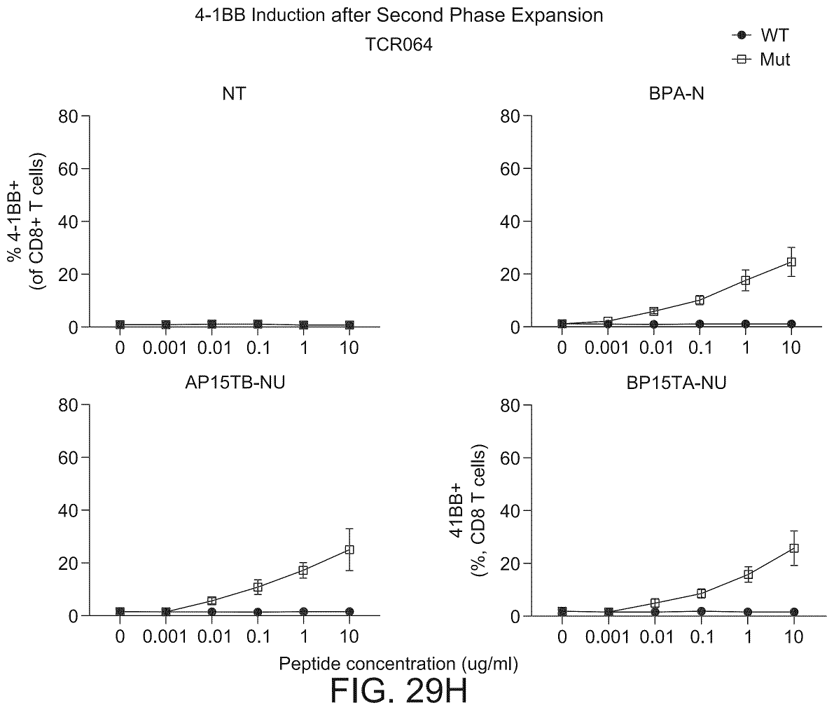

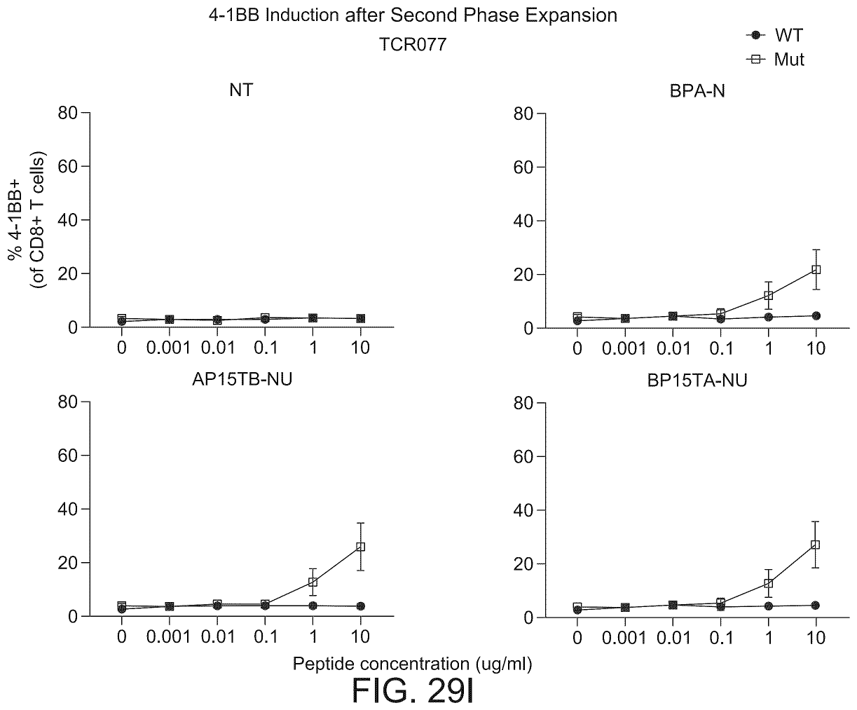

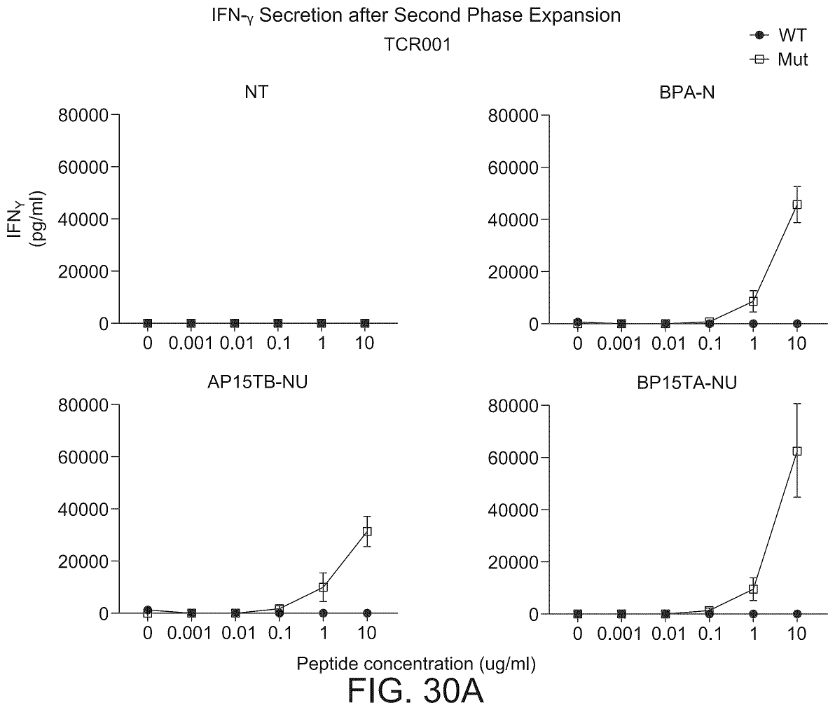

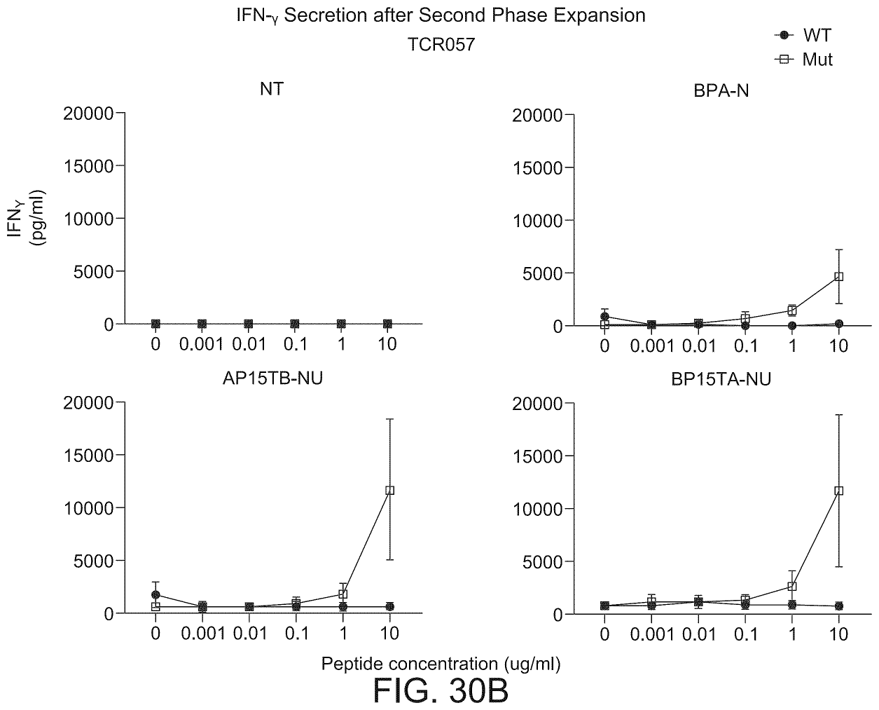

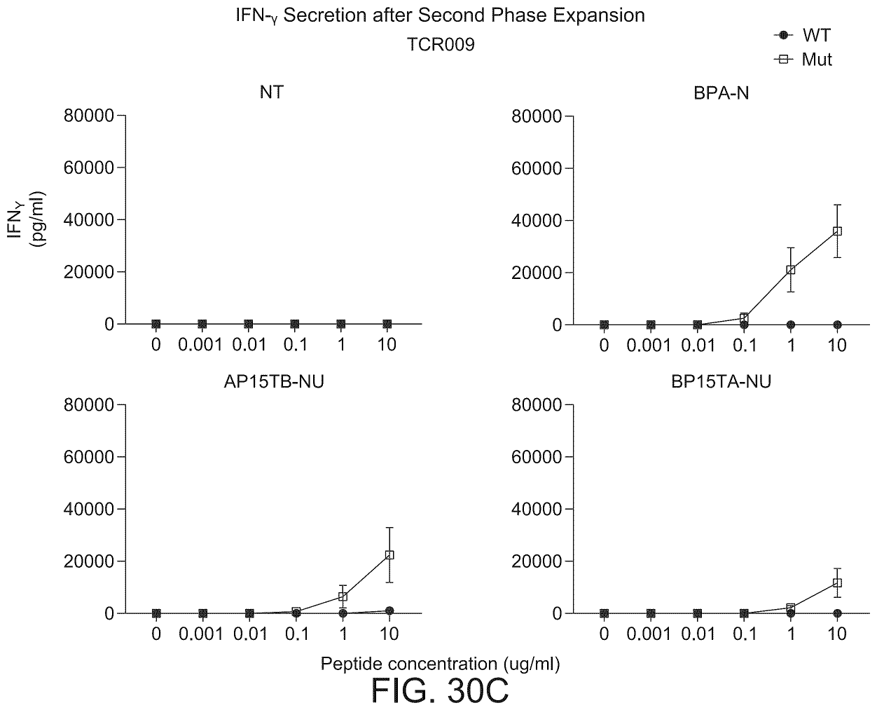

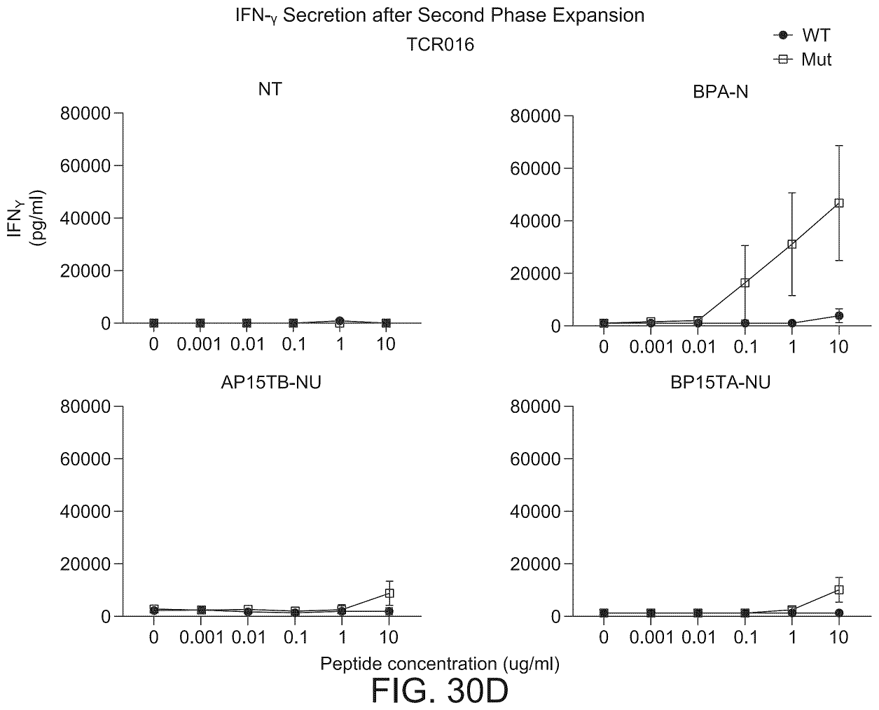

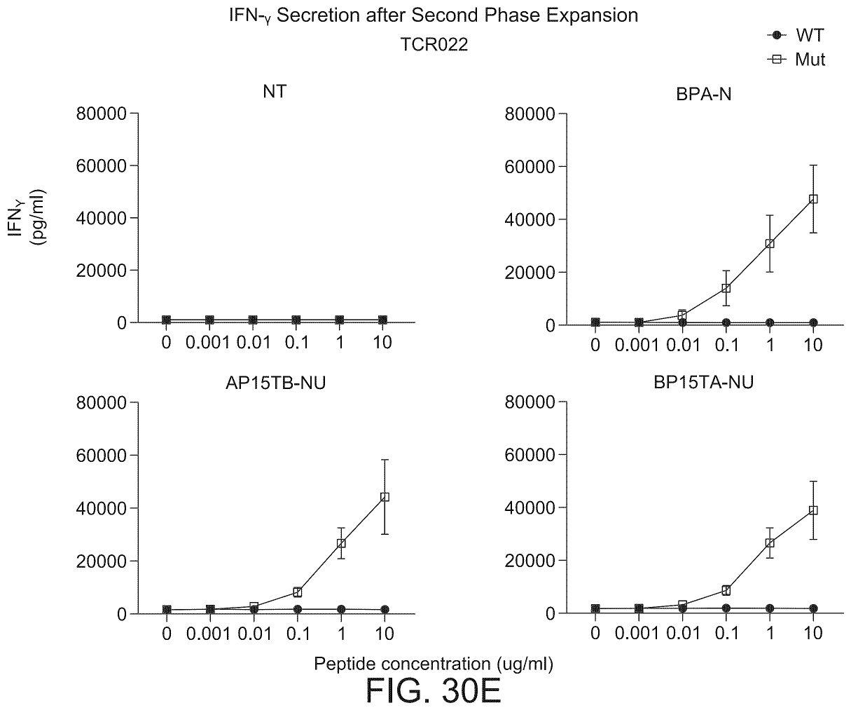

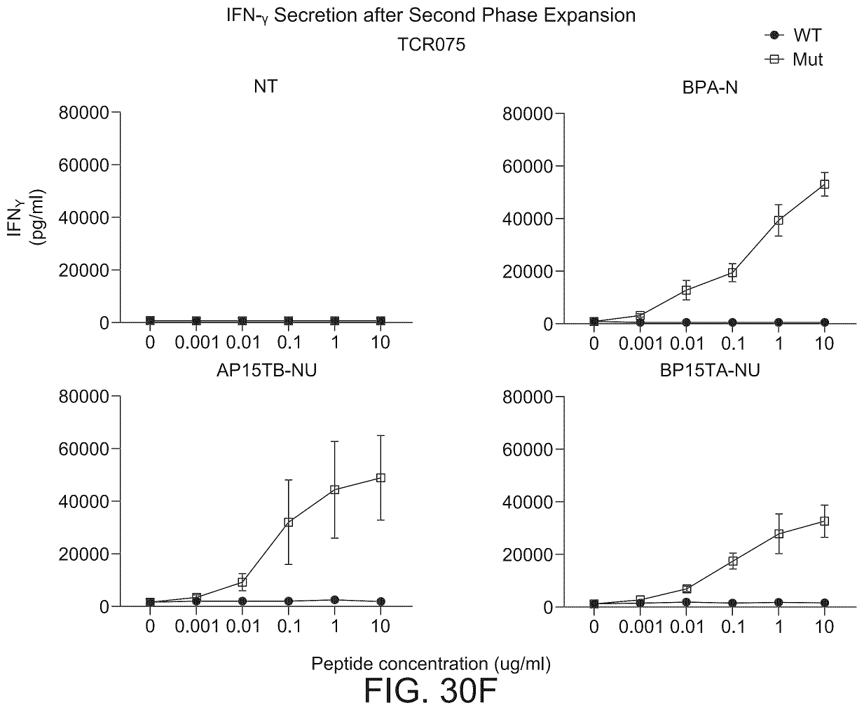

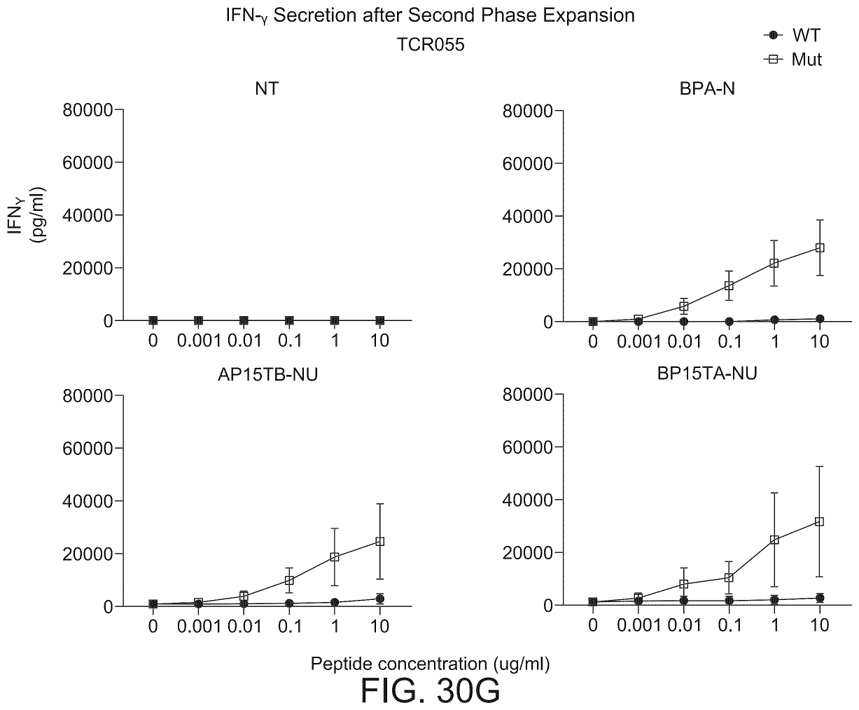

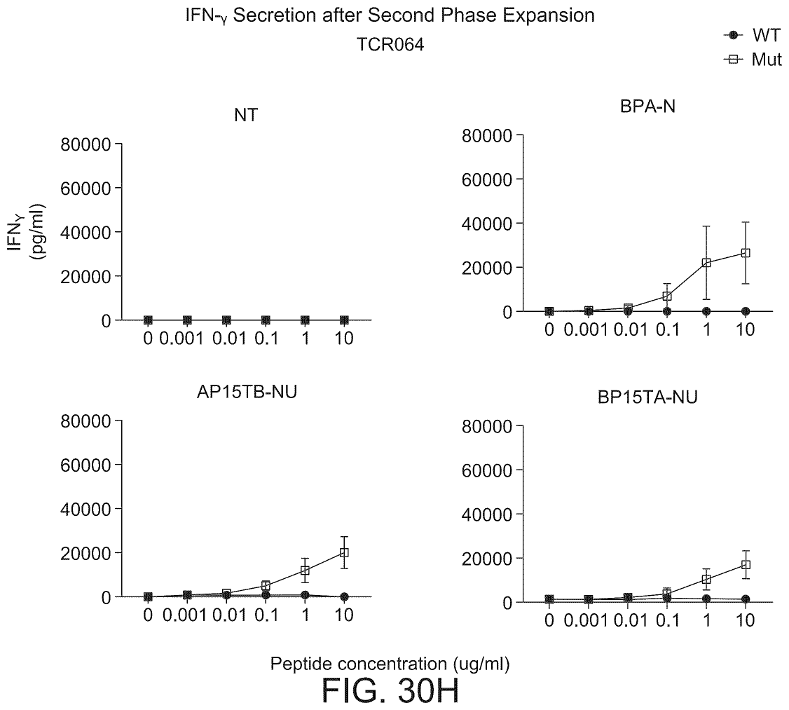

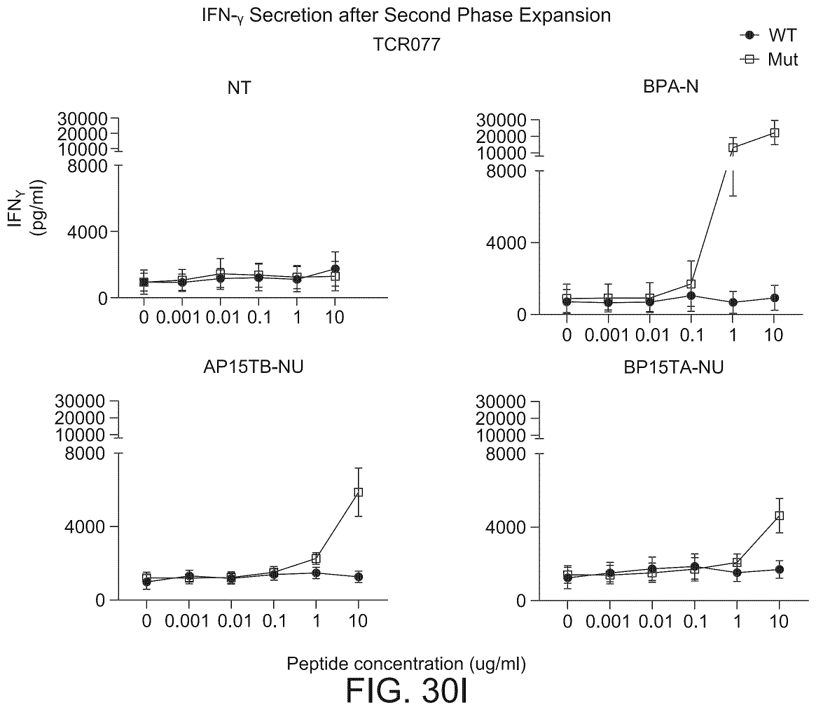

To assess activation of the generated TCR T-cells, overnight co-culture of the generated TCR T-cells with wild-type or mutant neoantigen pulsed DCs (HLA matched) was performed after 16 days second phase expansion and 4-1BB induction and IFN-γ secretion were measured. Induction of 4-1BB on CD3+CD8+ cells was detected with 4-1BB antibody. Secretion of IFN-γ measured with the ELISA antibody pair. The 4-1BB induction results are shown in FIG. 29A-29I and IFN-γ secretion results are shown in FIG. 30A-30I. The results demonstrate that when challenged with their cognate neoantigen, mbIL-15 TCR-T cells were highly avid and specific to the target neoantigens as measured by upregulation of 4-1BB co-stimulatory receptor and secretion of IFN-γ with negligible recognition of wild type sequences.

All data from electroporation to the second expansion phase of TCR vetting demonstrated that tricistronic system, expressing TCRα, TCRβ and mbIL15 with one plasmid successfully generated mbIL15 TCR-T cells and the features of the generated mbIL15 TCR-T cells are comparable to TCR-T cells in terms of transgene expression, cell growth, and functional specificity (4-1BB induction and IFN-γ secretion).

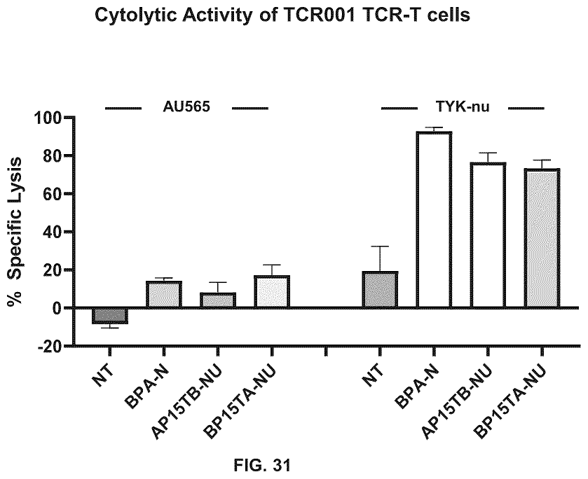

Cytolytic activity of TCR-T cells was assessed for T cells electroporated with polycistronic plasmids encoding TCR001+/−mbIL15 generated as described above (overnight recovery+11 days first phase expansion+11 days second phase expansion) and then harvested and frozen on Day 22. On experimental day, frozen Day 22 TCR-T cells were thawed and recovered for 3 days in media containing 3000 U/ml of IL-2. Then, the recovered TCR-T cells were incubated with AU565 (Mut+HLA−) or Tyk-nu (Mut+HLA+) cells. After overnight incubation, the remaining T cells were extensively washed, and the extent of viable cells left in the culture after TCR-specific cytolysis was measured using the CellTiter Glo luminescence-based assay. The results are shown in FIG. 31.

Specific lysis was calculated from background subtracted values as:

$$= [ \frac{\begin{matrix}( “ Tumor \& TCR - T ” value - \\ ( “ TCR - T ” Value + Mean “ Tumor ” Value ) )\end{matrix}}{\begin{matrix}“ TCR - T ” Value - \\ ( “ TCR - T ” Value + Mean “ Tumor ” Value )\end{matrix}} ] \times 100$$

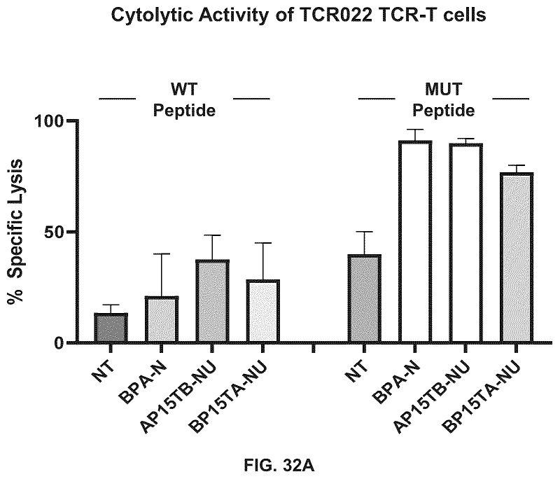

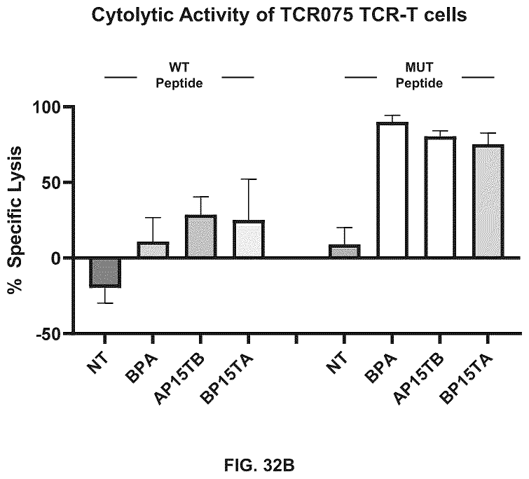

Cytolytic activity of TCR-T cells was also assessed for T cells electroporated with polycistronic plasmids encoding TCR022+/−mbIL15 or TCR075+/−mbIL15 generated as described above (overnight recovery+11 days first phase expansion+11 days expansion) and then harvested and frozen on Day 22. On experimental day, frozen Day 22 TCR-T cells were thawed and recovered for 3 days in media containing 3000 U/ml of IL-2. Meantime, Saos-2 cells were plated in 96 well plate. After overnight incubation, HLA 11:01 plasmid was transfected into the Saos-2 cells and on the following day, WT or MUT neoantigenic peptides (1 ug/ml) were loaded on the transfected Saos-2 cells for 2 hours. Then, the recovered TCR-T cells were incubated with the resulting Saos-2 cells overnight. After the overnight incubation, the remaining T cells were extensively washed, and the extent of viable cells left in the culture after TCR-specific cytolysis was measured using the CellTiter Glo luminescence-based assay. The results are shown in FIG. 32A-32B.

Specific lysis was calculated from background subtracted values as:

$$= [ \frac{\begin{matrix}( “ Peptide loaded tumor \& TCR - T ” value - \\ ( “ TCR - T ” Value + Mean “ Peptide loaded Tumor ” Value )\end{matrix}}{\begin{matrix}“ TCR - T ” Value - ( “ TCR - T ” Value - \\ Mean “ Peptide loaded tumor ” Value )\end{matrix}} ] \times$$

The cytolytic activity data demonstrated that mbIL15 TCR-T cells generated using the tricistronic system exhibited specific lytic activity against target tumor cells although the efficacy of these cells was less effective compared to that of TCR-T cells.

The long-term withdrawal (LTWD) assay was performed to examine the transgene expression, survival and activation of T cells cultured under cytokine-free conditions. The LTWD assay was performed as follows. The engineered T cells at Day 22 (post first phase and second phase expansion) were transferred to T25 flask and cultured for 4 weeks in cytokine-free media (50:50). 50% of media was exchanged every week. For the control TCR only (BA) groups, cells were treated with 300 U/ml IL-2 twice a week while exchanging the 50% of media. Flow data were acquired using an NovoCyte Quanteon flow cytometer system (Agilent) and analyzed with FlowJo software (version 10.7.1; TreeStar, Ashland, OR). (Data n=4, pooled from 2 independent experiments)

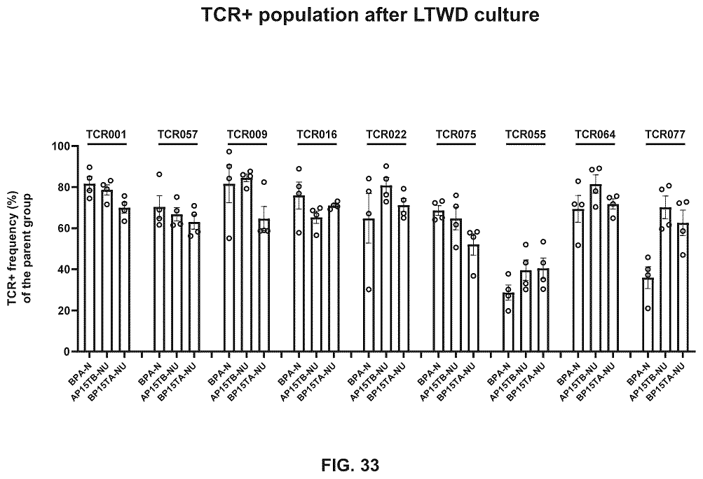

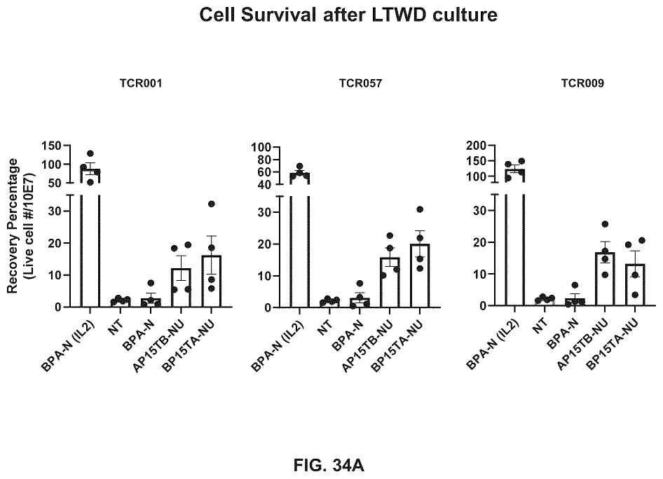

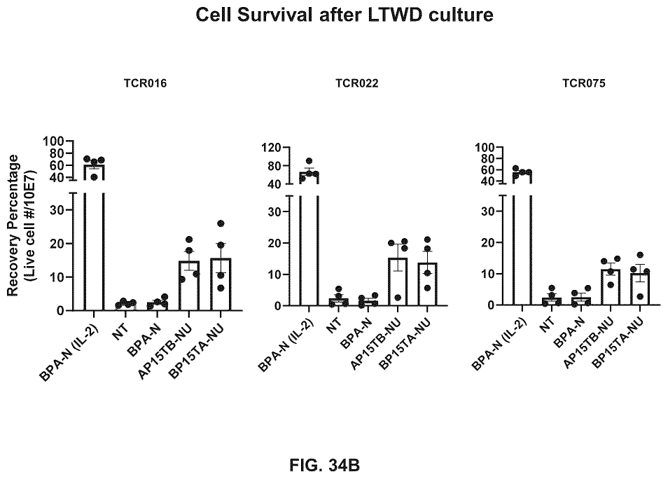

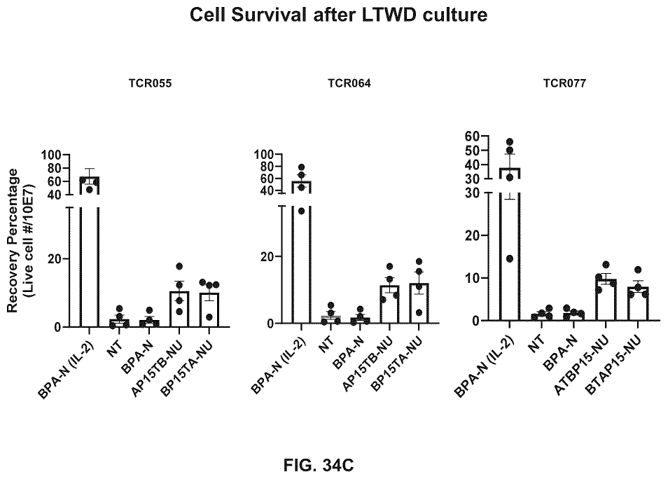

After 4 weeks LTWD incubation, the expression of mTCR was detected on CD3+ gated population with mouse TCR beta antibody (FIG. 33) and cell count and viability were accessed (FIG. 34A-34C). This mTCR expression and cell count data demonstrated no significant difference between mbIL15 TCR-T cells generated with the different polycistronic plasmids. The number of viable cells decreased after long-term cytokine withdrawal in all groups, but cells from the groups co-expressing mbIL15 and TCR survived 5-6 fold more compared to cells from the TCR only groups.

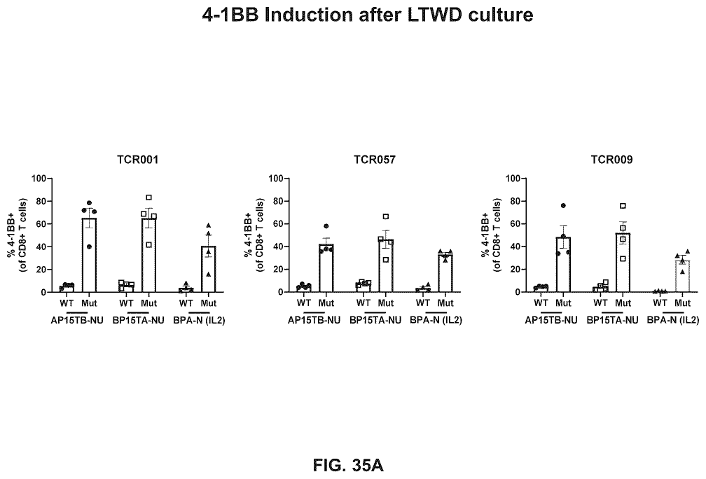

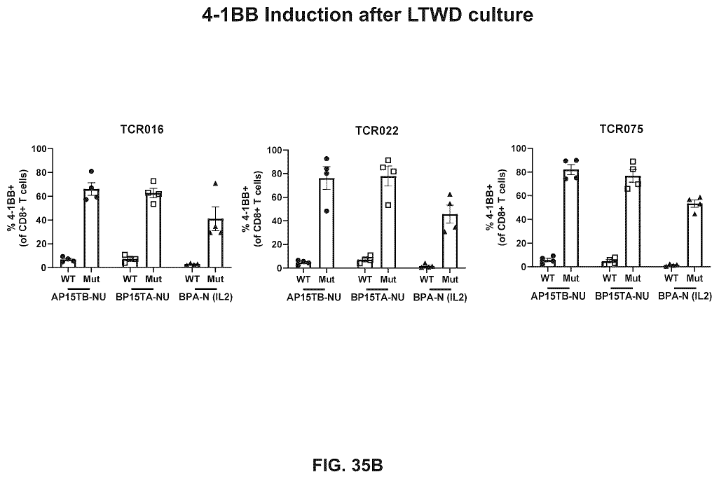

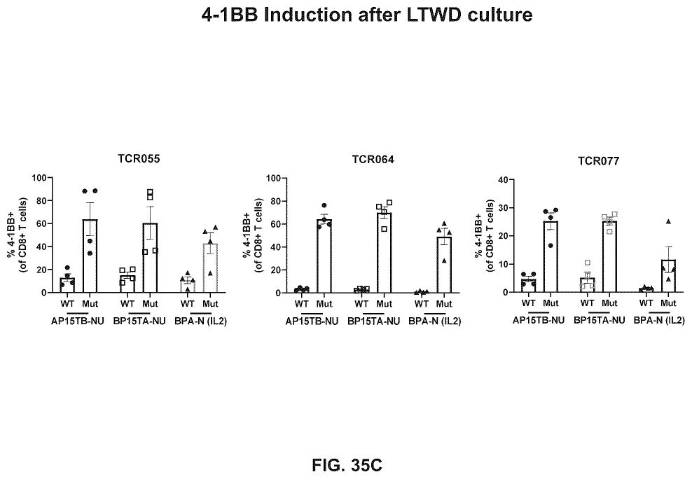

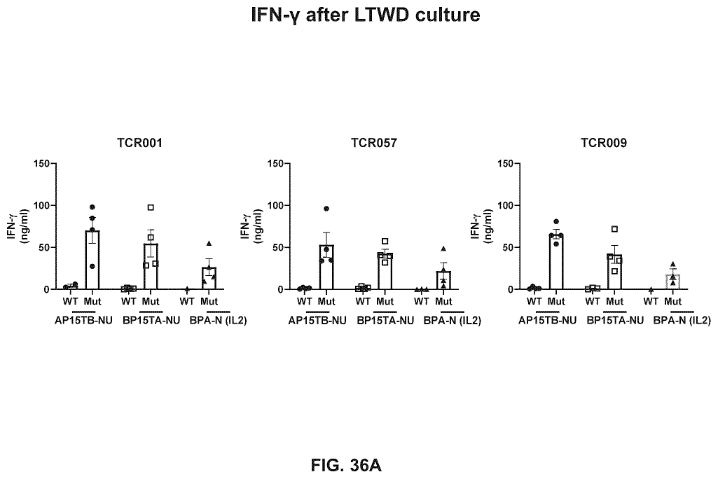

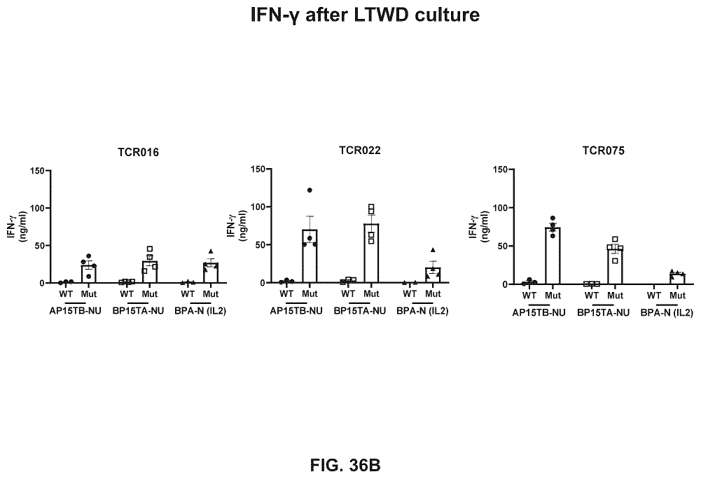

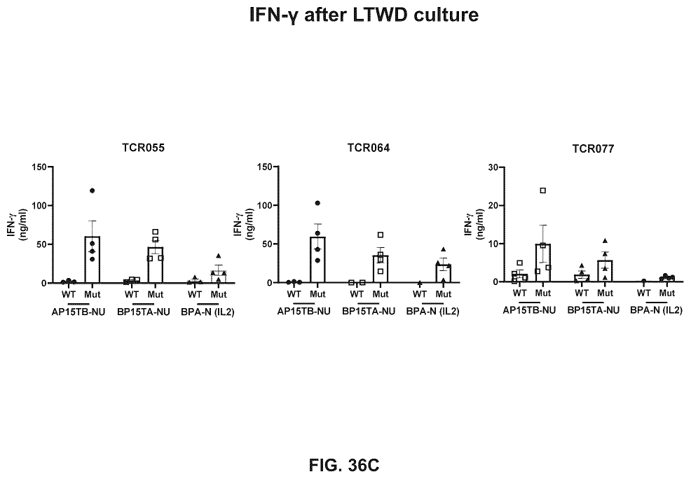

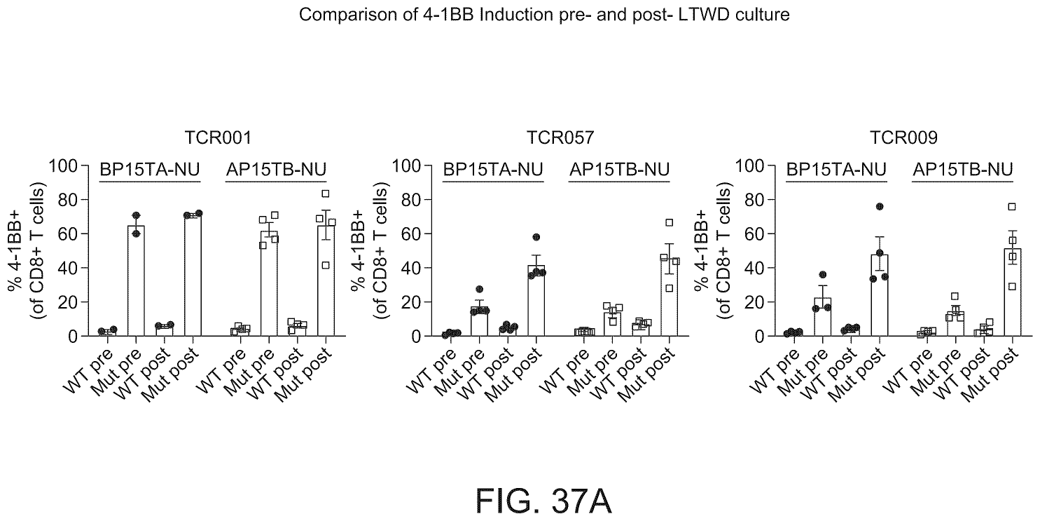

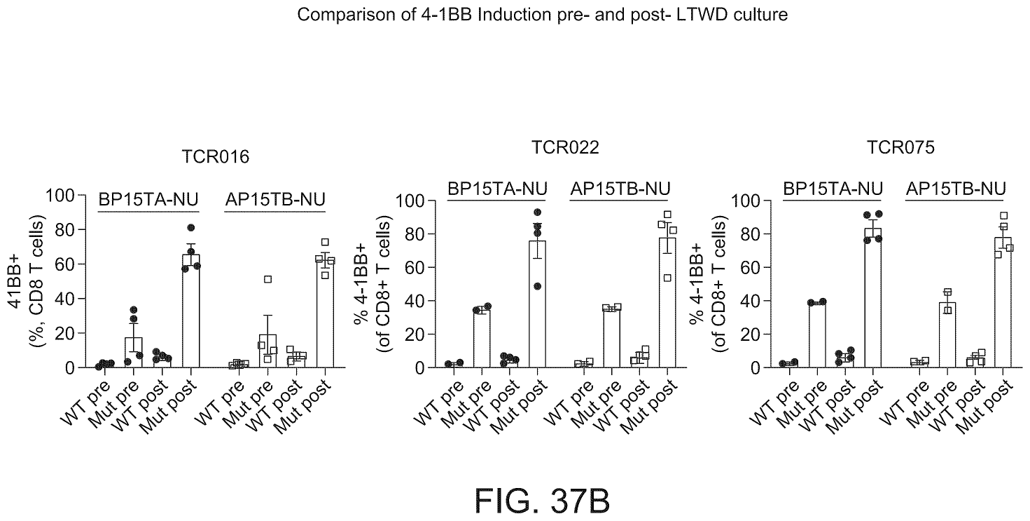

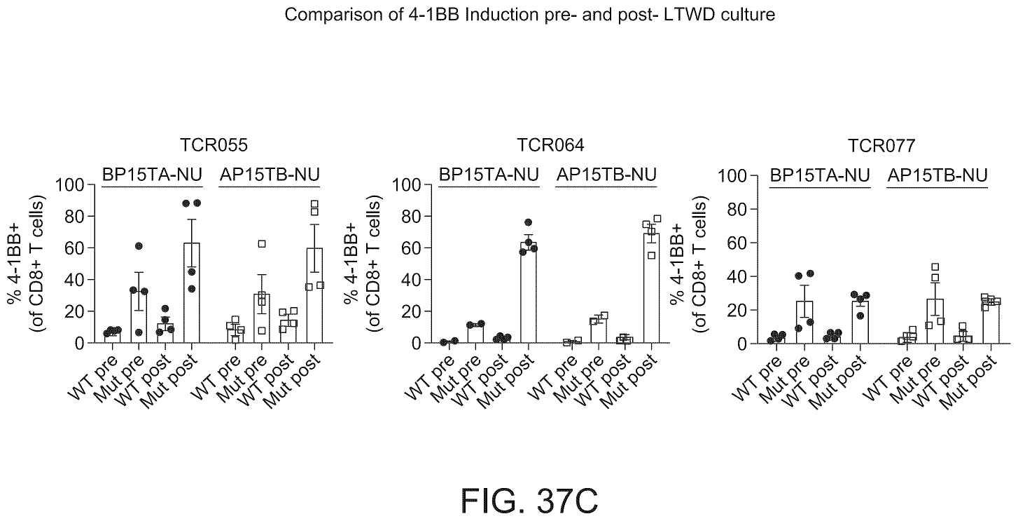

The activation of TCR-T cells after LTWD culture was assessed by 4-1BB induction and IFN-γ secretion after overnight co-culture with wild-type or mutant neoantigen pulsed DCs (HLA matched). As described above, induction of 4-1BB on CD3+CD8+ cells was detected with 4-1BB antibody (FIG. 35A-35C) and IFN-γ secretion was measured with the ELISA antibody pair (FIG. 36A-36C). A comparison of 4-1BB induction assessed for T cells harvested at Day 27 and after LTWD is shown in FIG. 37A-37C. These data demonstrate that mbIL15 TCR-T cells which survived LTWD culture are still functional and were more strongly activated than cells from TCR only groups. The data also demonstrate that after 4 week of cytokine withdrawal (LTWD), mbIL15 TCR-T cells showed even more potent induction of 4-1BB compared to those cells after the second expansion phase.

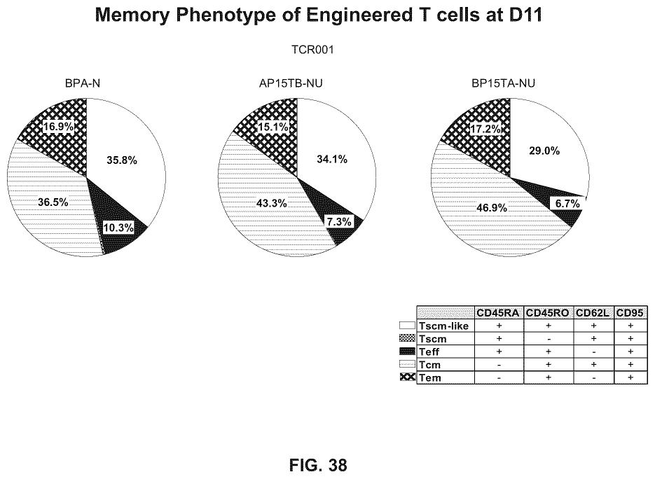

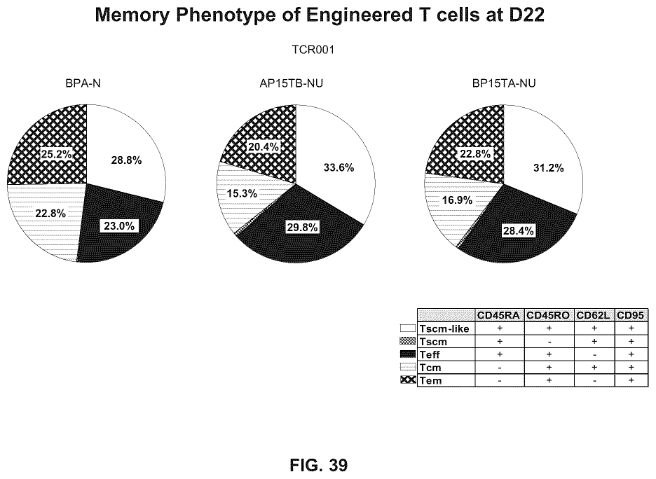

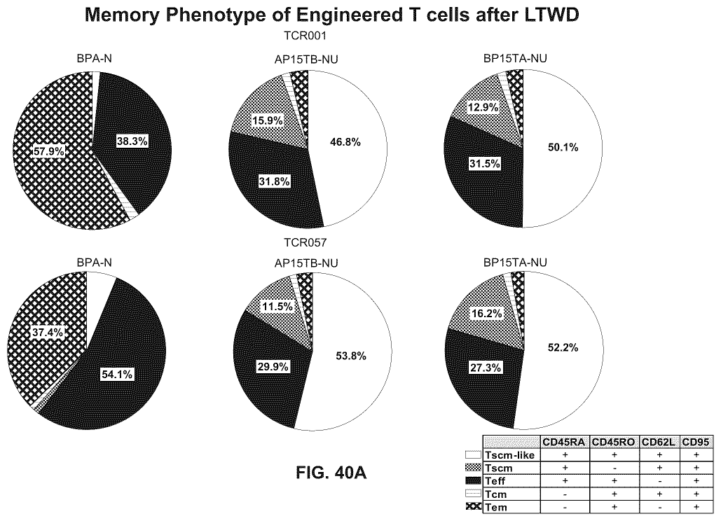

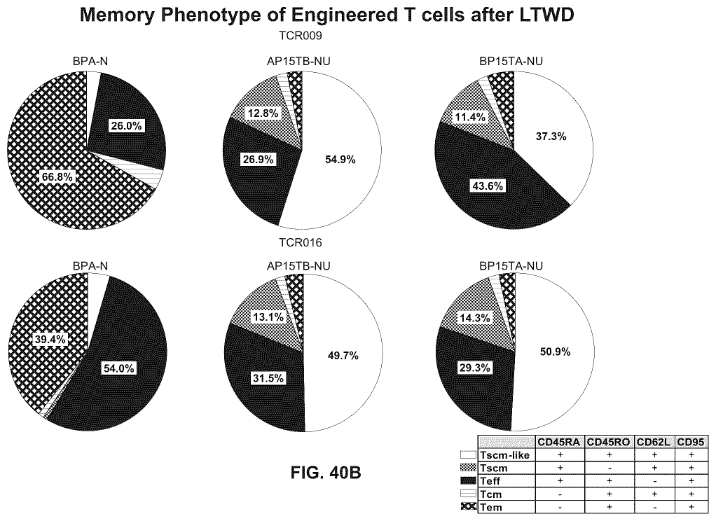

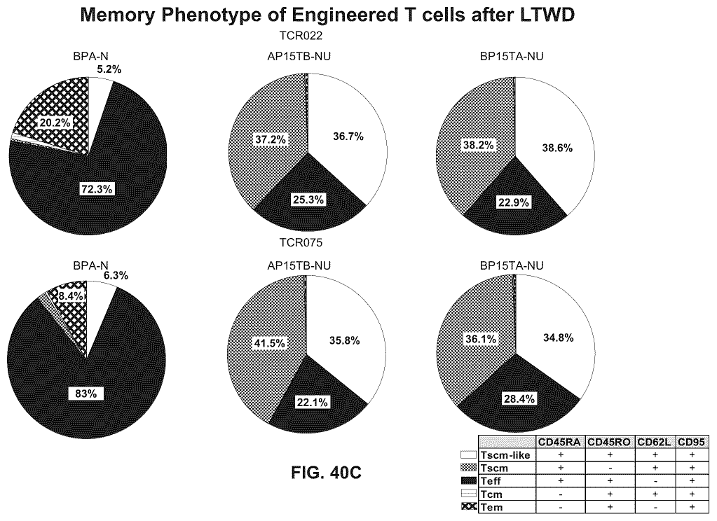

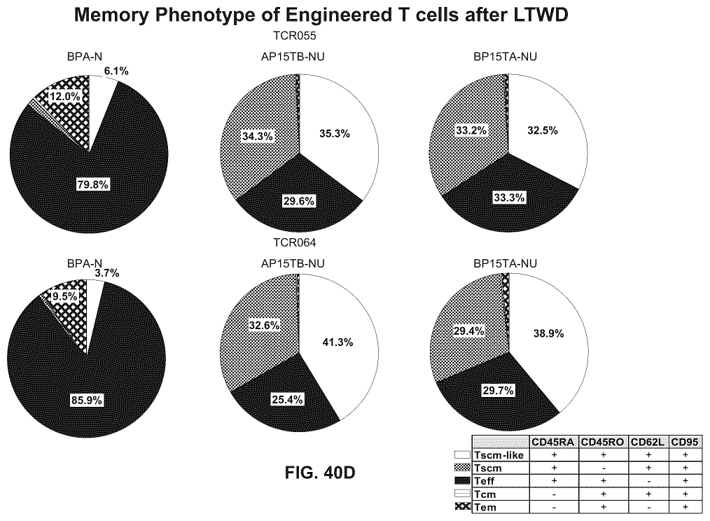

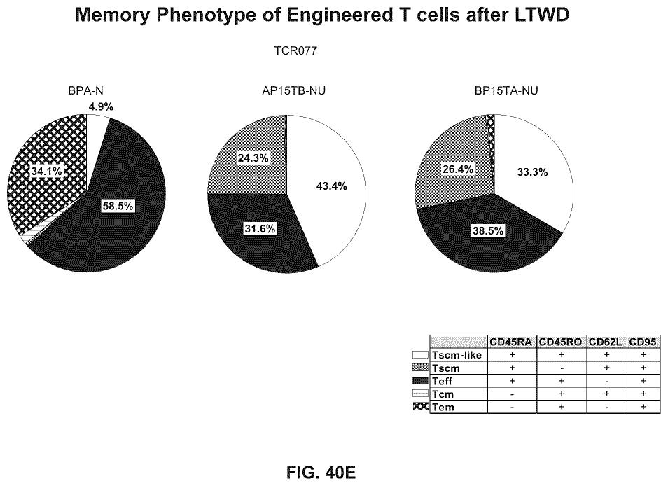

Memory phenotype of TCR-T cells electroporated with different polycistronic vectors was also assessed. T cell memory subsets are defined as: CD45RA+CD45RO+CD62L+CD95+=stem cell memory-like (Tscm-like); CD45RA+CD45RO-CD62L+CD95+=stem cell memory (Tscm); CD45RA-CD45RO+CD62L+CD95+=central memory (Tcm); CD45RA-CD45RO+CD62L-CD95+=effector memory (Tem). T cell effector (Teff) are defined as CD45RA+CD45RO+CD62L-CD95+. The data in Tables E14 and E15 and representative pie charts in FIGS. 38-40 show the mean frequency of live CD3⁺ T cell memory and effector subsets at day 11 post-expansion (Table E14 & FIG. 38), day 22 post-expansion (Table E15 & FIG. 39), and after 4 weeks of LTWD culture (FIGS. 40A-40E) in cells transposed with the tested plasmids.

TABLE E14
Memory phenotype of engineered T cells at D11.

| | | % Tscm- | % | % | % | % |
|---|---|---|---|---|---|---|
| Plasmid | | like | Tscm | Teff | Tcm | Tem |
| 1 | TCR001-BPA-N | 35.8 | 0.5 | 10.3 | 36.5 | 16.9 |
| | TCR001-AP15TB-NU | 34.1 | 0.2 | 7.3 | 43.3 | 15.1 |
| | TCR001-BP15TA-NU | 29.0 | nd | 6.7 | 46.9 | 17.2 |
| 2 | TCR057-BPA-N | 37.1 | 0.4 | 12.3 | 31.6 | 18.6 |
| | TCR057-AP15TB-NU | 33.7 | 0.1 | 7.7 | 43.5 | 15.0 |
| | TCR057-BP15TA-NU | 31.4 | 0.2 | 6.8 | 45.5 | 16.1 |
| 3 | TCR009-BPA-N | 38.3 | 0.6 | 11.8 | 29.9 | 19.4 |
| | TCR009-AP15TB-NU | 34.8 | 0.1 | 7.1 | 44.0 | 14.0 |
| | TCR009-BP15TA-NU | 28.5 | 0.2 | 6.3 | 47.7 | 17.3 |
| 4 | TCR016-BPA-N | 40.2 | 0.6 | 12.6 | 30.0 | 16.6 |
| | TCR016-AP15TB-NU | 32.8 | 0.4 | 6.8 | 44.8 | 15.2 |
| | TCR016-BP15TA-NU | 32.1 | 0.3 | 7.0 | 45.1 | 15.5 |
| 5 | TCR022-BPA-N | 58.1 | 2.9 | 7.5 | 25.6 | 5.9 |
| | TCR022-AP15TB-NU | 55.1 | 0.8 | 2.4 | 38.7 | 3.0 |
| | TCR022-BP15TA-NU | 56.9 | 0.7 | 2.3 | 37.4 | 2.7 |
| 6 | TCR075-BPA-N | 59.7 | 2.8 | 8.5 | 23.9 | 5.1 |
| | TCR075-AP15TB-NU | 53.5 | 0.7 | 2.1 | 40.6 | 3.1 |
| | TCR075-BP15TA-NU | 56.8 | 0.8 | 2.5 | 37.2 | 2.7 |
| 7 | TCR055-BPA-N | 55.6 | 2.5 | 8.2 | 27.8 | 5.9 |
| | TCR055-AP15TB-NU | 56.0 | 0.9 | 2.5 | 38.1 | 2.5 |
| | TCR055-BP15TA-NU | 56.6 | 0.8 | 2.5 | 37.3 | 2.8 |
| 8 | TCR064-BPA-N | 57.3 | 3.3 | 8.2 | 25.5 | 5.7 |
| | TCR064-AP15TB-NU | 53.3 | 0.8 | 2.5 | 40.1 | 3.3 |
| | TCR064-BP15TA-NU | 54.0 | 0.6 | 2.4 | 39.6 | 3.4 |
| 9 | TCR077-BPA-N | 63.8 | 3.0 | 5.3 | 22.0 | 5.8 |
| | TCR077-AP15TB-NU | 50.7 | 0.2 | 3.7 | 41.2 | 4.2 |
| | TCR077-BP15TA-NU | 50.7 | 0.2 | 4.3 | 40.4 | 4.4 |

TABLE E15
Memory phenotype of engineered T cells at D22.

| | | % Tscm- | % | % | % | % |
|---|---|---|---|---|---|---|
| Plasmid | | like | Tscm | Teff | Tcm | Tem |
| 1 | TCR001-BPA-N | 28.8 | 0.2 | 23.0 | 22.8 | 25.2 |
| | TCR001-AP15TB-NU | 33.6 | 0.9 | 29.8 | 15.3 | 20.4 |
| | TCR001-BP15TA-NU | 31.2 | 0.7 | 28.4 | 16.9 | 22.8 |
| 2 | TCR057-BPA-N | 31.2 | 0.2 | 22.7 | 23.1 | 22.8 |
| | TCR057-AP15TB-NU | 33.5 | 0.4 | 28.1 | 18.0 | 20.0 |
| | TCR057-BP15TA-NU | 34.4 | 0.7 | 28.5 | 16.5 | 19.9 |
| 3 | TCR009-BPA-N | 26.3 | 0.1 | 19.0 | 27.7 | 26.9 |
| | TCR009-AP15TB-NU | 34.7 | 0.2 | 29.4 | 15.3 | 19.4 |
| | TCR009-BP15TA-NU | 27.5 | 0.8 | 32.9 | 12.7 | 26.1 |
| 4 | TCR016-BPA-N | 30.7 | 0.1 | 22.1 | 23.3 | 23.8 |
| | TCR016-AP15TB-NU | 35.3 | 0.8 | 29.6 | 14.1 | 20.2 |
| | TCR016-BP15TA-NU | 33.4 | 0.8 | 28.9 | 14.7 | 22.2 |
| 5 | TCR022-BPA-N | 28.7 | 0.1 | 15.3 | 31.8 | 24.1 |
| | TCR022-AP15TB-NU | 32.3 | 0.7 | 22.1 | 25.1 | 19.8 |
| | TCR022-BP15TA-NU | 30.4 | 0.5 | 22.8 | 24.1 | 22.2 |
| 6 | TCR075-BPA-N | 26.9 | 0.2 | 15.6 | 33.4 | 23.9 |
| | TCR075-AP15TB-NU | 31.7 | 0.7 | 22.6 | 22.3 | 22.7 |
| | TCR075-BP15TA-NU | 31.4 | 0.5 | 23.9 | 22.2 | 22.0 |
| 7 | TCR055-BPA-N | 22.9 | 0.2 | 14.9 | 35.1 | 26.9 |
| | TCR055-AP15TB-NU | 28.8 | 0.5 | 27.5 | 19.7 | 23.5 |
| | TCR055-BP15TA-NU | 28.8 | 0.4 | 25.7 | 20.2 | 24.9 |
| 8 | TCR064-BPA-N | 24.1 | 0.1 | 13.9 | 34.3 | 27.6 |
| | TCR064-AP15TB-NU | 28.7 | 0.3 | 21.1 | 25.8 | 24.1 |
| | TCR064-BP15TA-NU | 28.8 | 0.6 | 22.5 | 23.6 | 24.5 |
| 9 | TCR077-BPA-N | 34.3 | 0.3 | 27.8 | 19.2 | 18.4 |
| | TCR077-AP15TB-NU | 37.8 | 2.2 | 34.8 | 10.7 | 14.6 |
| | TCR077-BP15TA-NU | 37.8 | 1.9 | 36.4 | 10.1 | 13.8 |

Memory phenotype data shows the kinetics of TCR-T memory and effector differentiation. The addition of mbIL15 to TCR-T cells resulted in changes to the memory phenotype in the expanded product to contain fewer central memory cells (Tcm) and more effector (Teff) and stem cell memory (Tscm) populations relative to conventional TCR-T cells. After 4 weeks of culture in presence of IL-2, TCR-T cells predominantly differentiated into Teff cells. TCR-T cells expressing mbIL15 cultured for 4 weeks in the absence of cytokines differentiated into 3 main subsets: Teff, Tscm-like and Tscm cells. These results suggest that mbIL15 is sufficient to guide T cell differentiation to the Tscm phenotype.

Conclusions: mbIL15 TCR-T cells were successfully generated using 18 different constructs (2 different orientations; AP15 TB and BP15TA×9 TCRs). The addition of mbIL15 to TCR-T cells resulted in changes to the memory phenotype in the expanded product to contain fewer central memory cells (Tcm) and more effector (Teff) and stem cell memory (Tscm) populations relative to conventional TCR-T cells. Furthermore, long-term withdrawal of cytokine support (LTWD) demonstrated survival of a fraction of mbIL15 TCR-T cells which was significantly higher than survival of TCR-T cells lacking mbIL15. Functional and phenotypic evaluation of the persistent mbIL15 TCR-T cells revealed that they retained their functional neoantigen specificity and potency while displaying a preponderance of Tscm TCR-T cells capable of regenerating TCR-T cell effector pools. This suggested that mbIL15 TCR-T cells could likely establish long-lived tumor-specific TCR-T cells that potentially overcome suppression by the tumor microenvironment or other negative regulators. This non-clinical data supports clinical application of mbIL15 TCR-T cell platform and provides evidence that this strategy could result in improved efficacy for cancer treatment.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

What is claimed:

1. A method of expanding a population of electroporated T cells comprising culturing the electroporated T cells with a first culture medium comprising one or more cytokines.

2. The method of claim 1, wherein the electroporated T cells are contacted with the first culture medium within 12 hours of electroporation.

3. The method of claim 1 or 2, wherein the one or more cytokines are selected from the group consisting of IL-7, IL-15, and IL-21.

4. The method of any one of claims 1 to 3, wherein the first culture medium further comprises an exogenous glutathione precursor.

5. The method of claim 4, wherein the glutathione precursor is N-acetylcysteine (NAC).

6. The method of any one of claims 1 to 5, wherein the first culture medium comprises IL-15.

7. The method of any one of claims 1 to 6, wherein the first culture medium comprises IL-7.

8. The method of any one of claims 1 to 7, wherein the first culture medium comprises IL-21.

9. The method of any one of claims 1 to 8, wherein the first culture medium comprises IL-7 and IL-21.

10. The method of any one of claims 1 to 9, wherein the electroporated T cells are electroporated prior to culturing with the first culture medium.

11. The method of claim 10, wherein the electroporated T cells are cultured in the first culture medium for 6-12 hours after electroporation.

12. The method of any one of claims 1 to 11 further comprising culturing the T cells with a second culture medium, wherein the second culture medium comprises one or more cytokines selected from the group consisting of IL-7, IL-12, and IL-21.

13. The method of claim 12, wherein the second culture medium comprises IL-7, IL-12, and IL-21.

14. The method of claim 12 or 13, wherein IL-21 is added to the second culture medium every 2 to 3 days.

15. The method of any one of claims 12 to 14, wherein at least one of the cytokines selected from the group consisting of IL-7 and IL-12 are added to the second culture medium only on the first day of culturing.

16. The method of claim 15, wherein IL-7 and IL-12 are added to the second culture medium only on the first day of culturing.

17. The method of any one of claims 12 to 16, wherein the T cells are cultured in the second culture medium after being cultured in the first culture medium.

18. The method of any one of claims 12 to 17, wherein the T cells are cultured in the second culture medium for 11 to 13 days.

19. The method of any one of claims 1 to 18, further comprising culturing the T cells with a third culture medium, wherein the third culture medium comprises one or more cytokines selected from the group consisting of IL-2 and IL-21.

20. The method of claim 19, wherein the third culture medium comprises IL-2.

21. The method of claim 19 or 20, wherein the third culture medium comprises IL-21.

22. The method of any one of claims 19 to 21, wherein the third culture medium further comprises IL-12.

23. The method of any one of claims 19 to 22, wherein the third culture medium further comprises an exogenous glutathione precursor.

24. The method of claim 23, wherein the exogenous glutathione precursor is NAC.

25. The method of any one of claims 19 to 24, wherein the third culture medium comprises IL-12, IL-21 and NAC.

26. The method of any one of claims 19 to 25, wherein the third culture medium comprises IL-2, IL-12, IL-21 and NAC.

27. The method of any one of claims 21 to 26, wherein IL-21 is added to the third culture medium every 2 to 3 days.

28. The method of any one of claims 20 or 22 to 26, wherein IL-2 is added to the third culture medium every 3 to 4 days.

29. The method of any one of claims 20 or 22 to 27, wherein IL-2 is present in the third culture medium in an amount from 30 U/ml to 3000 U/ml.

30. The method of any one of claims 22 to 29, wherein the IL-12 is added to the third culture medium only on the first day of culturing.

31. The method of any one of claims 19 to 30, wherein the T cells are cultured in the third culture medium after being cultured in the second culture medium.

32. The method of any one of claims 19 to 31, wherein the T cells are cultured in the third culture medium for 11 to 13 days.

33. The method of any one of claims 1 to 32, wherein the first, second and/or third culture media further comprise a TCR agonist.

34. The method of claim 33, wherein the TCR agonist is a CD3 agonist.

35. The method of any one of claims 1 to 34, wherein the first, second and/or third culture media further comprise an agonist of a T cell costimulatory molecule.

36. The method of claim 35, wherein the agonist of a T cell costimulatory molecule is a CD28 agonist.

37. The method of any one of claims 1 to 36, wherein the first, second and/or third culture media further comprise a nanomatrix.

38. The method of claim 37, wherein the TCR agonist and/or the T cell costimulatory molecule is associated with the nanomatrix.

39. The method of any one of claims 1 to 38, further comprising culturing the cells with feeder cells.

40. A population of engineered T cells manufactured according to the method of any one of claims 1 to 39.

41. The population of engineered T cells of claim 40, wherein more than 10% of the engineered T cells in the population comprise one or more of the following: an exogenous TCR or functional fragment thereof, and an exogenous membrane-bound IL-15.

42. The population of engineered T cells of claim 40, wherein more than 2% of the engineered T cells in the population co-express an exogenous TCR or functional fragment thereof and an exogenous membrane-bound IL-15.

43. A population of engineered T cells, wherein more than 10% of the engineered T cells in the population comprise an exogenous TCR or functional fragment thereof, and wherein more than 20% of the population of engineered T cells are CCR7+/CD45RO+.

44. A population of engineered T cells, wherein more than 10% of the engineered T cells in the population comprise an exogenous TCR or functional fragment thereof, and wherein more than 40% of the population of engineered T cells are CD95+/CD62L+.

45. The population of engineered T cells of claim 44, wherein the population of engineered T cells comprise more than 5%, 10%, 15%, 20%, 25%, 30%, 35%, 40%, 45% or 50% CD45RA+CD45RO-CD62L+CD95+ cells.

46. The population of engineered T cells of claim 44, wherein the population of engineered T cells comprise more than 5%, 10%, 15%, 20%, 25%, 30%, 35%, 40%, 45% or 50% CD45RA+CD45RO+CD62L+CD95+ cells.

47. A population of cells comprising a polycistronic expression cassette comprising:
a. a first cistron comprising a polynucleotide sequence that encodes a fusion protein that comprises IL-15, or a functional fragment or functional variant thereof, and IL-15Rα, or a functional fragment or functional variant thereof;
b. a second cistron comprising a polynucleotide sequence that encodes a TCR beta chain comprising a Vβ region and a Cβ region; and
c. a third cistron comprising a polynucleotide sequence that encodes a TCR alpha chain comprising a Vα region and a Cα region.

48. The population of cells of claim 47, wherein the population of cells are T cells that comprise more than 5%, 10%, 15%, 20%, 25%, 30%, 35%, 40%, 45% or 50% CD45RA+CD45RO-CD62L+CD95+ cells.

49. The population of cells of claim 47, wherein the population of cells are T cells that comprise more than 5%, 10%, 15%, 20%, 25%, 30%, 35%, 40%, 45% or 50% CD45RA+CD45RO+CD62L+CD95+ cells.
